(19) United States
(12) Patent Application Publication
(10) Pub. No.: US 2025/0109410 A1
(43) Pub. Date: Apr. 3, 2025

(54) Stable Cell Lines for Inducible Production of rAAV Virions

(71) Applicant: Shape Therapeutics Inc., Seattle, WA (US)

(72) Inventors: Kenneth Prentice, Seattle, WA (US); Sandhya Pande, Seattle, WA (US); Lauriel Earley, Tacoma, WA (US); Lindsay Nicole Deis, Lynwood, WA (US); RIchard Thomas Sullivan, Seattle, WA (US)

(21) Appl. No.: 18/834,546

(22) PCT Filed: Jan. 31, 2023
(86) PCT No.: PCT/US2023/061667

§ 371 Date: Jul. 30, 2024

Related U.S. Application Data
(60) Provisional application No. 63/437,553, filed on Jan. 6, 2023.
(60) Provisional application No. 63/427,037, filed on Nov. 21, 2022.
(60) Provisional application No. 63/404,434, filed on Sep. 7, 2022.
(60) Provisional application No. 63/316,308, filed on Mar. 3, 2022.
(60) Provisional application No. 63/305,662, filed on Feb. 1, 2022.

(51) Int. Cl. C12N 15/86; C12N 5/073

(52) U.S. Cl. CPC C12N 15/86; C12N 5/0603; C12N 2750/14143; C12N 2750/14152; C12N 2800/30; C12N 2830/002; C12N 2840/203

(57) ABSTRACT

Described herein are polynucleotide constructs for inducible production of rAAV virions. Also provided are stable cell lines for inducible expression of rAAV virions that include a pay load poly nucleotide.

### CROSS-REFERENCE TO RELATED APPLICATIONS

This application claims priority to U.S. Provisional Application No. 63/437,553, filed on Jan. 6, 2023; U.S. Provisional Application No. 63/427,037, filed on Nov. 21, 2022; U.S. Provisional Application No. 63/404,434, filed on Sep. 7, 2022; U.S. Provisional Application No. 63/316,308, filed on Mar. 3, 2022; and U.S. Provisional Application No. 63/305,662, filed on Feb. 1, 2022, the disclosures of which applications are herein incorporated by reference in their entirety.

### INCORPORATION BY REFERENCE OF SEQUENCE LISTING XML

A Sequence Listing is provided herewith as Sequence Listing XML, “SHPE-006WO_SEQ_LIST,” created on Jan. 31, 2023, and having a size of 444,705 bytes. The contents of the Sequence Listing XML are incorporated by reference herein in their entirety.

### INTRODUCTION

Recombinant adeno-associated virus (rAAV) is the preferred vehicle for in vivo gene delivery. AAV has no known disease associations, infects dividing and non-dividing cells, rarely if ever integrates into the mammalian cell genome, and can persist essentially for the lifetime of infected cells as a transcriptionally active nuclear episome. The FDA has recently approved several rAAV gene therapy products and many other rAAV-based gene therapy and gene editing products are in development.

The most widely used method for producing rAAV virions is based on the helper-virus-free transient transfection of multiple plasmids, typically a triple transfection, into adherent cell lines. Although there is ongoing investment to increase production capacity, current AAV manufacturing processes are inefficient and expensive. In addition, they result in variable product quality, with low levels of encapsidation of a payload, such as a therapeutic payload.

There is, therefore, a need for improved methods for producing rAAV products. Any such solution must address the toxicity to the host production cell due to constitutive expression of AAV Rep protein and the toxicity to the host production cell due to constitutive expression of adenoviral helper protein.

### SUMMARY

Disclosed herein are stable mammalian cell lines, wherein the cells are capable of conditionally producing recombinant AAV (rAAV) virions within which are packaged an expressible payload. Disclosed herein are constructs that are capable of conditionally producing recombinant AAV (rAAV) virions within which are packaged an expressible payload, when introduced into a cell. The constructs may or may not be integrated into the genome of the cell.

Further provided herein is a stable mammalian cell line, wherein the cells are capable of conditionally producing recombinant AAV (rAAV) virions within which are packaged an expressible payload; and wherein a population of virions produced by the stable cell are more homogenous than a population of virions produced by an otherwise comparable cell producing rAAV virions upon transient transfection.

Further provided herein is a stable mammalian cell line and constructs, wherein the cells are capable of conditionally producing recombinant AAV (rAAV) virions within which are packaged an expressible payload; and production of virions is inducible upon addition of a triggering agent.

Further provided herein is a stable mammalian cell line and constructs, wherein the cells are capable of conditionally producing recombinant AAV (rAAV) virions within which are packaged an expressible payload; and production of virions is not conditioned on the presence of a plasmid within the cell.

In some aspects, a composition comprising one or more nucleic acids which together comprises: (i) a first recombinant nucleic acid sequence encoding an AAV Rep protein and an AAV Cap protein; and (ii) a second recombinant nucleic acid sequence encoding one or more adenoviral helper proteins, wherein when the one or more nucleic acids are integrated into the nuclear genome of a mammalian cell the AAV Rep protein, the AAV Cap protein, and/or the one or more adenoviral helper proteins are conditionally expressible and thereby conditionally produce recombinant AAV (rAAV) virions. In some embodiments, the conditional expression of the AAV Rep protein, the AAV Cap protein, and/or the one or more adenoviral helper proteins is controlled by one or more excisable elements present in the one or more nucleic acids. In some embodiments, the one or more excisable elements comprise one or more introns and/or one or more exons. In some embodiments, the first recombinant nucleic acid sequence encodes: a) a first part of the AAV Rep protein coding sequence; b) the second part of the AAV Rep protein coding sequence; c) an excisable element between the first part of the AAV Rep protein coding sequence and the second part of the AAV Rep protein coding sequence; and d) the AAV Cap protein coding sequence. In some embodiments, the excisable element comprises: a) a first spacer segment comprising a first intron, b) a second spacer segment comprising a coding sequence of a detectable marker; and c) a third spacer segment comprising a second intron, and wherein the first spacer segment and the third spacer segment are capable of being excised by endogenous cellular machinery of a mammalian cell. In some embodiments, the excisable element comprises from 5′ to 3′: a) a 5′ splice site; b) a first spacer segment comprising a first intron; c) a second spacer segment comprising: i) a first lox sequence; ii) a 3′ splice site; iii) an exon; iv) a stop signaling sequence; and v) a second lox sequence; and d) a third spacer segment comprising a second intron. In some embodiments, the detectable marker is a luminescent marker, a radiolabel or a fluorescent marker, optionally a fluorescent marker which is GFP, EGFP, RFP, CFP, BFP, YFP, or mCherry. In some embodiments, a) the first spacer segment comprises a nucleic acid sequence having at least 80% identity to SEQ ID NO: 1; and/or b) the second spacer segment comprises a nucleic acid sequence having at least 80% identity to SEQ ID NO: 2; and/or c) the third spacer segment comprises a nucleic acid sequence having at least 80% identity to SEQ ID NO: 3. In some embodiments, the second spacer segment is capable of being excised by a Cre polypeptide. In some embodiments, the expression of the AAV Rep protein and/or the AAV Cap protein is driven by native promoters. In some embodiments, wherein: a) the native promoters P5 and/or P19 drive the expression of the AAV Rep protein; and/or b) the native promoter P40 drives the expression of the AAV Cap protein. In some embodiments, the second recombinant nucleic acid sequence encodes: a) one or more adenoviral helper proteins; b) a conditionally self-excising element; and c) an inducible promoter; wherein, once integrated into the nuclear genome of a mammalian cell, the expression of the one or more adenoviral helper protein coding sequences is under the control of the conditionally self-excising element and the inducible promoter. In some embodiments, the one or more adenoviral helper proteins comprise E2A and E4. In some embodiments, the self-excising element comprises a sequence which encodes a polypeptide, preferably a recombinase polypeptide, more preferably a Cre polypeptide. In some embodiments, the polypeptide encoded by the self-excising element is conditionally expressible and is expressed only in the presence of a triggering agent. In some embodiments, the triggering agent is a hormone, preferably tamoxifen. In some embodiments, the inducible promoter is a Tet inducible promoter. In some embodiments, the second recombinant nucleic acid sequence further comprises a sequence that encodes a Tet responsive activator protein, preferably Tet-on-3G. In some embodiments, the expression of Tet-On 3G activator protein is driven by an EF1alpha promoter. In some embodiments, the second recombinant nucleic acid sequence comprises a sequence with at least 80% homology, at least 90% homology, at least 95% homology, at least 99% homology, or a sequence identical to SEQ ID NO: 11 or SEQ ID NO: 12. In some embodiments, the one or more nucleic acids further comprises a nucleic acid sequence encoding a VA RNA sequence. In some embodiments, the expression of VA RNA is constitutive. In some embodiments, the expression of VA RNA is inducible. In some embodiments, the VA RNA sequence comprises one or more mutations in the VA RNA internal promoter, preferably G16A and G60A. In some embodiments, the expression of VA RNA is driven by a EF1alpha promoter, a U6 promoter, or a U7 promoter. In some embodiments, the expression of VA RNA is driven by a U6 promoter or a U7 promoter. In some embodiments, the U6 promoter or the U7 promoter comprises: a) a first part of a U6 or U7 promoter sequence, b) a stuffer sequence, and c) a second part of a U6 or U7 promoter sequence, and wherein the stuffer sequence is capable of being excised by a Cre polypeptide. In some embodiments, a serotype of the AAV Cap protein is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 and AAVhu68. In some embodiments, the serotype is an AAV5 and the Cap protein that comprises one or more mutations or insertions. In some embodiments, the one or more recombinant nucleic acids further encode a third recombinant nucleic acid sequence encoding a payload, optionally wherein the payload is: (a) a polynucleotide payload, such as a guide RNA for RNA editing, a guide RNA for Cas protein-directed DNA editing, a tRNA suppressor, or a gene for replacement gene therapy; or (b) a protein such as a therapeutic antibody or a vaccine immunogen. In some embodiments, the one or more recombinant nucleic acids comprise one or more mammalian cell selection elements. In some embodiments, one or more of the mammalian cell selection elements encodes an antibiotic resistance gene, optionally a blasticidin resistance gene. In some embodiments, one or more of the mammalian cell selection elements is an auxotrophic selection element which encodes an active protein. In some embodiments the auxotrophic selection element is glutamine synthetase (GS), thymidylate synthase (TYMS), phenylalanine hydroxylase (PAH), or dihydrofolate reductase (DHFR). In some embodiments, one or more of the mammalian cell selection elements is a first auxotrophic selection element which encodes an inactive protein that requires expression of a second inactive protein from a second auxotrophic selection coding sequence for activity. In some embodiments, the first auxotrophic selection coding sequence encodes for DHFR Z-Cter (SEQ ID NO: 5) activity, and/or wherein the second auxotrophic selection coding sequence encodes for DHFR Z-Nter (SEQ ID NO: 4). In some embodiments, a) the first recombinant nucleic acid comprises a mammalian cell selection element which encodes an antibiotic resistance gene, preferably a blasticidin resistance gene; and b) the second recombinant nucleic acid comprises a first auxotrophic selection element which encodes an inactive protein that requires expression of a second inactive protein from a second auxotrophic selection coding sequence for activity; and c) the third recombinant nucleic acid comprises the second auxotrophic selection element which encodes the inactive protein that requires expression of the first inactive protein from the first auxotrophic selection coding sequence for activity; and wherein in (i) or (ii) the first auxotrophic selection coding sequence encodes for DHFR Z-Cter (SEQ ID NO: 5), and the second auxotrophic selection coding sequence encodes for DHFR Z-Nter (SEQ ID NO: 4) or wherein the first auxotrophic selection coding sequence encodes for DHFR Z-Nter (SEQ ID NO: 4), and the second auxotrophic selection coding sequence encodes for DHFR Z-Cter (SEQ ID NO: 5). In some embodiments, elements of the previous embodiments are capable of being in one or more separate constructs, in any combination, wherein the one or more constructs are capable of conditionally producing recombinant AAV (rAAV) virions within which are packaged an expressible payload, when introduced into a cell.

In some aspects, disclosed herein is a mammalian cell wherein the nuclear genome of the cell comprises a plurality of integrated recombinant nucleic acid constructs which together encode for a recombinant adeno-associated virus (rAAV) virions, wherein the rAAV virions can be conditionally expressed from the cell. In some embodiments, the plurality of integrated recombinant nucleic acid constructs comprise the one or more recombinant nucleic acids of any one of previous embodiment, wherein the AAV Rep protein, the AAV Cap protein and/or the adenoviral helper proteins can be conditionally expressed from the cell. In some embodiments, the cell line expresses adenoviral helper proteins E1A and E1B. In some embodiments, the plurality of integrated recombinant nucleic acid constructs comprise: (i) a first integrated polynucleotide construct comprising: a) a first part of an AAV Rep protein coding sequence; b) a second part of an AAV Rep protein coding sequence; c) an excisable element between the first part of the AAV Rep protein coding sequence and the second part of the AAV Rep protein coding sequence, wherein the excisable element comprises: i) a first spacer segment comprising a first intron; ii) a second spacer segment comprising a coding sequence of a detectable marker, wherein the second spacer segment is capable of being excised by a Cre polypeptide; and iii) a third spacer segment comprising a second intron; and d) an AAV Cap protein coding sequence; wherein the AAV Rep protein and the AAV Cap protein is driven by the native promoters P5, P19, and P40; (ii) a second integrated polynucleotide construct comprising a) a conditionally expressible VA RNA coding sequence which comprises a mutation in the VA RNA internal promoter, wherein the expression of VA RNA is driven by a U6 or a U7 promoter, optionally wherein the VA RNA sequence comprises G16A and G60A mutations; b) one or more adenoviral helper protein coding sequences, wherein the adenoviral helper proteins are E2A and E4; c) a conditionally self-excising element which encodes a Cre polypeptide which translocates to the nucleus and self-excises only in the presence of a triggering agent which is tamoxifen, and d) an inducible promoter which is a Tet inducible promoter, and wherein the expression of the one or more adenoviral helper protein coding sequences is under the control of the conditionally self-excising element and the inducible promoter; and (iii) a third integrated polynucleotide construct comprising encodes for the payload, wherein the payload is a polynucleotide payload.

In some aspects, a method of producing a population of rAAV virions comprises: (a) culturing the cell of any one of the embodiments disclosed herein in conditions which allow for the expression of the rAAV virions; and (b) isolating the rAAV virions from the cell culture.

In some embodiments, the prepurification rAAV viral genome (VG) to viral particle (VG:VP) ratio of greater than 0.5. In some embodiments, the population of rAAV virions produced by the cell has: (a) a ratio of viral genomes to transduction units of about 500 to 1 to 1 to 1; and/or (b) a ratio of vector genomes to infectious unit of 100:1.

In some aspects, a method of preparing the cell of any one of the previous embodiments comprises: i) providing a mammalian cell and the one or more nucleic acids of any one of the previous embodiments; and ii) integrating the one or more nucleic acids of any one of the previous embodiments into the nuclear genome of the mammalian cell.

In some aspects, a method of preparing the cell of any one of the previous embodiments comprises providing a mammalian cell and the one or more nucleic acids of any one of the previous embodiments. In some embodiments, the one or more nucleic acids of any one of the previous embodiments integrates into the nuclear genome of the mammalian cell. In some embodiments, the one or more nucleic acids of any one of the previous embodiments do not integrate into the nuclear genome of the mammalian cell.

In some aspects, a population of rAAV virions produced by the method of any one of the previous embodiments. In some embodiments, the infectivity of the virions is at least 50% at an MOI of 10000.

In some aspects, a pharmaceutical composition comprising a population of rAAV virions according to any one of the previous embodiments, for use as a medicament, optionally for use in treating a monogenic disorder. In some embodiments, the population of rAAV virions according to any one of the previous embodiments or the pharmaceutical composition according to any one of the previous embodiments, for use as a medicament, optionally for use in treating a monogenic disorder. In some embodiments, the population of rAAV virions or the pharmaceutical composition for use according to any one of the previous embodiments, wherein the rAAV virions are administered at a dosage of 4×10¹⁴ or lower.

Also provided herein are cells comprising: a) a first polynucleotide construct coding for an AAV Rep protein and an AAV Cap protein; b) a second polynucleotide construct coding for one or more adenoviral helper proteins; wherein when the one or more nucleic acids are integrated into the nuclear genome of a mammalian cell the AAV Rep protein, the AAV Cap protein, and/or the one or more adenoviral helper proteins are conditionally expressible and thereby conditionally produce recombinant AAV (rAAV) virions.

In some embodiments, the second polynucleotide construct comprises a sequence coding for: a) one or more helper proteins; b) a self-excising element upstream of the one or more helper proteins; and c) an inducible promoter upstream of the self-excising element. In some embodiments, the self-excising element is operably linked to the inducible promoter. In some embodiments, expression of the self-excising element is driven by the inducible promoter.

In some embodiments, the inducible promoter is a tetracycline-responsive promoter element (TRE). In some embodiments, the TRE comprises Tet operator (tetO) sequence concatemers fused to a minimal promoter. In some embodiments, the minimal promoter is a human cytomegalovirus promoter. In some embodiments, the minimal promoter comprises a sequence having at least 70%, 80%, 90%, 95%, 99%, or 100% sequence identity to any one of SEQ ID NOs: 63-68. In some embodiments, the inducible promoter comprises a sequence having at least 70%, 80%, 90%, 95%, 99%, or 100% sequence identity to any one of SEQ ID NOs: 22, 46-48, or 50-62. In some embodiments, transcription is activated from the inducible promoter upon binding of an activator. In some embodiments, the activator binds to the inducible promoter in the presence of a first triggering agent. In some embodiments, the second polynucleotide construct further comprises a sequence coding for an activator. In some embodiments, the activator is operably linked to a constitutive promoter. In some embodiments, the constitutive promoter is EF1alpha promoter or human cytomegalovirus promoter. In some embodiments, the EF1alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the activator is reverse tetracycline-controlled transactivator (rTA) comprising a Tet Repressor binding protein (TetR) fused to a VP16 transactivation domain. In some embodiments, the rTA comprises four mutations in the tetR DNA binding moiety. In some embodiments, the rTA comprises a sequence having at least 70%, 80%, 90%, 95%, 99%, or 100% sequence identity to any one of SEQ ID NOs: 21, 40-45, or 69-85, or variants thereof.

In some embodiments, the inducible promoter is a cumate operator sequence. In some embodiments, the cumate operator sequence is downstream of a constitutive promoter. In some embodiments, the constitutive promoter is a human cytomegalovirus promoter. In some embodiments, the inducible promoter is bound by a cymR repressor in the absence of a first triggering agent. In some embodiments, the inducible promoter is activated in the presence of a first triggering agent. In some embodiments, the first triggering agent binds to the cymR repressor. In some embodiments, the second polynucleotide construct further comprises a cymR repressor. In some embodiments, the cymR repressor is operably linked to a constitutive promoter. In some embodiments, the constitutive promoter is EF1alpha promoter. In some embodiments, the EF1alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the first triggering agent is a cumate.

In some embodiments, the sequence coding for the self-excising element comprises a poly A sequence. In some embodiments, the self-excising element is a recombinase. In some embodiments, the recombinase is fused to a ligand binding domain. In some embodiments, the recombinase is Cre polypeptide or flippase polypeptide. In some embodiments, the Cre polypeptide is fused to a ligand binding domain. In some embodiments, the ligand binding domain is a hormone receptor. In some embodiments, the recombinase is a Cre-ERT2 polypeptide. In some embodiments, the self-excising element translocates to the nucleus in the presence of a second triggering agent. In some embodiments, the second triggering agent is an estrogen receptor ligand. In some embodiments, the second triggering agent is a selective estrogen receptor modulator (SERM). In some embodiments, the second triggering agent is tamoxifen. In some embodiments, the recombinase is flanked by recombination sites. In some embodiments, the recombination sites are lox sites or flippase recognition target (FRT) sites. In some embodiments, the lox sites are loxP sites.

In some embodiments, the one or more adenoviral helper proteins comprise E2A and E4. In some embodiments, the E2A is FLAG-tagged E2A. In some embodiments, the sequence coding for E2A and the sequence coding for E4 are separated by an internal ribosome entry site (IRES) or by P2A.

In some embodiments, the second polynucleotide construct further comprises a sequence coding for a selectable marker. In some embodiments, the selectable marker is an antibiotic resistance protein. In some embodiments, the selectable marker is an auxotrophic protein. In some embodiments, the selectable marker is a split intein linked to an N-terminus of the auxotrophic protein or split intein linked to a C-terminus of the auxotrophic protein. In some embodiments, the selectable marker is a leucine zipper linked to an N-terminus of the auxotrophic protein or leucine zipper linked to a C-terminus of the auxotrophic protein. In some embodiments, the auxotrophic protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal fragment of the split intein comprises at least 80%, 85%, 90%, 95%, 98%, 99%, or 100% sequence identity to SEQ ID NO: 140. In some embodiments, a C-terminal fragment of the split intein comprises at least 80%, 85%, 90%, 95%, 98%, 99%, or 100% sequence identity to SEQ ID NO: 141.

In some embodiments, the second polynucleotide construct further comprises a sequence coding for VA RNA. In some embodiments, the sequence coding for VA RNA is a transcriptionally dead sequence. In some embodiments, the sequence coding for VA RNA comprises at least two mutations in the internal promoter. In some embodiments, expression of VA RNA is driven by a U6 or U7 promoter. In some embodiments, the second polynucleotide construct further comprises upstream of the sequence coding for VA RNA gene sequence, from 5′ to 3′: a) a first part of a U6 or U7 promoter sequence; b) a first recombination site; c) a stuffer sequence; d) a second recombination site; and e) a second part of a U6 or U7 promoter sequence. In some embodiments, the stuffer sequence is excisable by the recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter.

In some embodiments, the first polynucleotide construct comprises: a) a sequence of a first part of a Rep gene; b) a sequence of a second part of the Rep gene; c) a sequence of a Cap gene; and d) an excisable element positioned between the first part of the sequence of Rep gene and the second part of the sequence of the Rep gene.

In some embodiments, the excisable element comprises a stop signaling sequence. In some embodiments, the excisable element comprises a rabbit beta globin intron. In some embodiments, the excisable element comprises an exon. In some embodiments, the excisable element comprises an intron and an exon. In some embodiments, the excisable element comprises an intron.

In some embodiments, two splice sites are positioned between the sequence of the first part of the Rep gene and the sequence of the second part of the Rep gene. In some embodiments, the two splice sites are a 5′ splice site and a 3′ splice site. In some embodiments, the 5′ splice site is a rabbit beta globin 5′ splice site. In some embodiments, the 3′ splice site is a rabbit beta globin 3′ splice site. In some embodiments, three splice sites are positioned between the sequence of the first part of the Rep gene and the sequence of the second part of the Rep gene. In some embodiments, the three splice sites are a 5′ splice site, a first 3′ splice site, and a second 3′ splice site. In some embodiments, a first 3′ splice site is a duplicate of the second 3′ splice site. In some embodiments, the first 3′ splice site is a rabbit beta globin 3′ splice site. In some embodiments, the second 3′ splice site is a rabbit beta globin 3′ splice site.

In some embodiments, the excisable element comprises a recombination site. In some embodiments, the recombination site is a lox site or FRT site. In some embodiments, the lox site is a loxP site.

In some embodiments, the excisable element comprises from 5′ to 3′: a) the 5′ splice site; b) a first recombination site; c) the first 3′ splice site; d) a stop signaling sequence; e) a second recombination site; and f) the second 3′ splice site.

In some embodiments, the excisable element comprises from 5′ to 3′: a) the 5′ splice site; b) a first spacer segment; c) a second spacer segment comprising: i) a first recombination site; ii) the first 3′ splice site; iv) a stop signaling sequence; and v) a second recombination site; and d) a third spacer segment comprising the second 3′ splice site. In some embodiments, the first spacer sequence comprises an intron. In some embodiments, the first spacer segment comprises a sequence having at least 70%, 80%, 90%, 95%, 99%, or 100% sequence identity to SEQ ID NO: 1. In some embodiments, the second spacer segment comprises a sequence having at least 70%, 80%, 90%, 95%, 99%, or 100% sequence identity to SEQ ID NO: 2. In some embodiments, the third spacer segment comprises a sequence having at least 70%, 80%, 90%, 95%, 99%, or 100% sequence identity to SEQ ID NO: 3. In some embodiments, the third spacer segment comprises an intron. In some embodiments, the first spacer segment and the third spacer segment are capable of being excised by endogenous cellular machinery. In some embodiments, the second spacer segment comprises an exon. In some embodiments, the second spacer segment further comprises a polyA sequence. In some embodiments, the poly A sequence is 3′ of the exon. In some embodiments, the polyA sequence comprises a rabbit beta globin (RBG) polyA sequence.

In some embodiments, the second spacer segment comprises from 5′ to 3′: a) a first recombination site; b) the first 3′ splice site; c) an exon; d) a stop signaling sequence; and e) a second recombination site. In some embodiments, the first recombination site is a first lox sequence and the second recombination site is a second lox sequence. In some embodiments, the first lox sequence is a first loxP sequence and a second lox sequence is a second loxP sequence. In some embodiments, the first recombination site is a first FRT site and the second recombination site is a second FRT site. In some embodiments, the stop signaling sequence is a termination codon of the exon or a polyA sequence. In some embodiments, the poly A sequence comprises a rabbit beta globin (RBG) polyA sequence. In some embodiments, the exon encodes a detectable marker or a selectable marker. In some embodiments, the detectable marker comprises a luminescent marker or a fluorescent marker. In some embodiments, the fluorescent marker is GFP, EGFP, RFP, CFP, BFP, YFP, or mCherry.

In some embodiments, the second spacer segment is excisable by a recombinase. In some embodiments, the recombinase is a Cre polypeptide or a Flippase polypeptide. In some embodiments, the Cre polypeptide is fused to a ligand binding domain. In some embodiments, the ligand binding domain is a hormone receptor. In some embodiments, the recombinase is a Cre-ERT2 polypeptide.

In some embodiments, the Rep gene codes for Rep polypeptides. In some embodiments, the Cap gene codes for Cap polypeptides. In some embodiments, transcription of the Rep gene and the Cap gene are driven by native promoters. In some embodiments, the native promoters comprise P5, P19, and P40.

In some embodiments, the Rep polypeptides are wildtype Rep polypeptides. In some embodiments, the Rep polypeptides comprise Rep78, Rep68, Rep52, and Rep40. In some embodiments, a truncated replication associated protein comprising a polypeptide expressed from the sequence of first part of a Rep gene and the exon is capable of being expressed in the absence of the recombinase.

In some embodiments, the Cap polypeptides are wildtype Cap polypeptides. In some embodiments, the Cap polypeptides are AAV capsid proteins. In some embodiments, the AAV capsid proteins comprise VP1, VP2, and VP3. In some embodiments, a serotype of the AAV capsid proteins is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68.

In some embodiments, the first polynucleotide construct further comprises a sequence coding for a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is DHFR, GS, TYMS, or PAH. In some embodiments, the selectable marker is a split intein linked to an N-terminus of the auxotrophic selection element or split intein linked to a C-terminus of the auxotrophic selection element. In some embodiments, the selectable marker is a split intein linked to an N-terminus of the active protein or split intein linked to a C-terminus of the active protein. In some embodiments, the selectable marker is a leucine zipper linked to an N-terminus of the auxotrophic selection element or leucine zipper linked to a C-terminus of the auxotrophic selection element. In some embodiments, the selectable marker is a leucine zipper linked to an N-terminus of the active protein or leucine zipper linked to a C-terminus of the active protein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance.

In some embodiments, the first polynucleotide construct comprises a sequence having at least 70%, 80%, 90%, 95%, 99%, or 100% sequence identity to any one of SEQ ID NO: 1-SEQ ID NO: 3, SEQ ID 6-SEQ ID NO: 8, SEQ ID NO: 32, SEQ ID NO: 90-SEQ ID NO: 99, SEQ ID NO: 101-SEQ ID NO: 109, SEQ ID NO: 112-SEQ ID NO: 131, or SEQ ID NO: 136-SEQ ID NO: 138. In some embodiments, the second polynucleotide construct has at least 70%, 80%, 90%, 95%, 99%, or 100% sequence identity to any one of SEQ ID NO: 9-SEQ ID NO: 19, SEQ ID 23-SEQ ID NO: 32, SEQ ID NO: 35, SEQ ID NO: 90-SEQ ID NO: 99, SEQ ID NO: 101-SEQ ID NO: 109, SEQ ID NO: 112-SEQ ID NO: 131, SEQ ID NO: 137, or SEQ ID NO: 138. In some embodiments, the Rep/Cap construct comprises SEQ ID NO: 145 downstream of the sequence encoding the AAV Cap proteins. In some embodiments, the Rep/Cap construct lacks SEQ ID NO: 145 downstream of the sequence encoding the AAV Cap proteins. In some embodiments, the Rep/Cap construct comprises a sequence having at least 70%, 80%, 90%, 95%, 99%, or 100% sequence identity to any one of SEQ ID NO: 1-SEQ ID NO: 3, SEQ ID 6-SEQ ID NO: 8, SEQ ID NO: 32, SEQ ID NO: 90-SEQ ID NO: 99, SEQ ID NO: 101-SEQ ID NO: 109, SEQ ID NO: 112-SEQ ID NO: 131, or SEQ ID NO: 136-SEQ ID NO: 138, but wherein these sequences lack SEQ ID NO: 145 downstream of the sequence encoding the AAV Cap proteins. In some embodiments, the first polynucleotide construct and the second polynucleotide construct are stably integrated in the cell's genome.

In some embodiments, the cell further comprises a payload construct, wherein the payload construct is a polynucleotide coding for a payload. In some embodiments, the payload construct comprises a sequence having at least 70%, 80%, 90%, 95%, 99%, or 100% sequence identity to SEQ ID NO: 33. In some embodiments, the payload construct has at least 70%, 80%, 90%, 95%, 99%, or 100% sequence identity to SEQ ID NO: 147. In some embodiments, the payload construct has at least 70%, 80%, 90%, 95%, 99%, or 100% sequence identity to SEQ ID NO: 149. In some embodiments, the payload construct has at least 70%, 80%, 90%, 95%, 99%, or 100% sequence identity to SEQ ID NO: 151. In some embodiments, the payload construct has at least 70%, 80%, 90%, 95%, 99%, or 100% sequence identity to SEQ ID NO: 153. In some embodiments, a plasmid comprises the payload construct and comprises a sequence having at least 70%, 80%, 90%, 95%, 99%, or 100% sequence identity to SEQ ID NO: 33. In some embodiments, a plasmid comprises the payload construct and comprises a sequence having at least 70%, 80%, 90%, 95%, 99%, or 100% sequence identity to SEQ ID NO: 147. In some embodiments, a plasmid comprises the payload construct and comprises a sequence having at least 70%, 80%, 90%, 95%, 99%, or 100% sequence identity to SEQ ID NO: 149. In some embodiments, a plasmid comprises the payload construct and comprises a sequence having at least 70%, 80%, 90%, 95%, 99%, or 100% sequence identity to SEQ ID NO: 151. In some embodiments, a plasmid comprises the payload construct and comprises a sequence having at least 70%, 80%, 90%, 95%, 99%, or 100% sequence identity to SEQ ID NO: 153. In some embodiments, the payload construct comprises a sequence of a payload flanked by ITR sequences. In some embodiments, the payload construct comprises a sequence having at least 70%, 80%, 90%, 95%, 99%, or 100% sequence identity to SEQ ID NO: 139. In some embodiments, expression of the sequence of the payload is driven by a constitutive promoter. In some embodiments, the constitutive promoter and sequence of the payload are flanked by ITR sequences. In some embodiments, the payload construct comprises a sequence having at least 70%, 80%, 90%, 95%, 99%, or 100% sequence identity to SEQ ID NO: 146. In some embodiments, the payload construct comprises a sequence having at least 70%, 80%, 90%, 95%, 99%, or 100% sequence identity to SEQ ID NO: 148. In some embodiments, the payload construct comprises a sequence having at least 70%, 80%, 90%, 95%, 99%, or 100% sequence identity to SEQ ID NO: 150. In some embodiments, the payload construct comprises a sequence having at least 70%, 80%, 90%, 95%, 99%, or 100% sequence identity to SEQ ID NO: 152.

In some embodiments, the sequence of the payload comprises a polynucleotide sequence coding for a gene. In some embodiments, the gene codes for a selectable marker or detectable marker. In some embodiments, the gene codes for a therapeutic polypeptide or transgene.

In some embodiments, the sequence of the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the payload construct is stably integrated into the genome of the cell.

In some embodiments, a plurality of the payload construct are stably integrated into the genome of the cell. In some embodiments, the plurality of the payload constructs are separately stably integrated into the genome of the cell. In some embodiments, the payload construct further comprises a sequence coding for a selectable marker or detectable marker outside of the ITR sequences. In some embodiments, the payload construct is integrated into the genome of the cell.

In some embodiments, a method for increasing production of rAAV virions from a cell, comprises amplifying expression of AAV Rep and capsid proteins, helper proteins, and/or payload in the cell, wherein the amplifying comprises:
increasing copy number of a polynucleotide construct comprising a sequence encoding one or more AAV Rep proteins and a sequence encoding one or more AAV cap proteins, a polynucleotide construct comprising a sequence encoding one or more AAV helper proteins, and/or a polynucleotide construct comprising a sequence encoding the payload; introducing an agent to amplify expression of the Rep/Cap genes, helper genes, and/or payload.

In some embodiments, the polynucleotide construct further comprises a selectable marker operably linked to an attenuated promoter. In some embodiments, the increasing copy number of the polynucleotide construct comprises culturing the cell under conditions that select for the presence of the selectable marker, thereby producing the cell comprising an increased copy number of the polynucleotide construct compared to the polynucleotide construct further comprising a selectable marker operably linked to a nonattenuated promoter. In some embodiments, the attenuated promoter is an attenuated EF1alpha promoter and the nonattenuated promoter is an EF1alpha promoter; optionally, wherein the attenuated EF1alpha promoter is SEQ ID NO: 132 and the EF1alpha promoter is SEQ ID NO: 133. In some embodiments, the polynucleotide construct further comprises a mutated selectable marker having decreased enzymatic activity compared to an unmutated selectable marker. In some embodiments, the increasing copy number of the polynucleotide construct comprises culturing the cell under conditions that select for the presence of the mutated selectable marker, thereby producing the cell comprising an increased copy number of the polynucleotide construct compared to the polynucleotide construct further comprising the unmutated selectable marker. In some embodiments, the mutated selectable marker is a mutated GS and the unmutated selectable marker is GS; optionally, wherein the mutated GS having a R324C, R324S, or R341C mutation as compared to SEQ ID NO: 112 and the GS is SEQ ID NO: 112; optionally, wherein the mutated GS is SEQ ID NO: 142, SEQ ID NO: 143, or SEQ ID NO: 144. In some embodiments, the polynucleotide construct further comprises a selectable marker. In some embodiments, the increasing copy number of the polynucleotide construct comprises culturing the cell under conditions that select for the presence of the selectable marker and in the presence of an inhibitor of the selectable marker, thereby producing the cell comprising an increased copy number of the polynucleotide construct compared to the polynucleotide construct further comprising the selectable marker cultured in the absence of the inhibitor of the selectable marker. In some embodiments, the polynucleotide construct is any polynucleotide construct as described herein.

Also provided herein are methods of producing a stable cell line comprising expanding a cell described above.

Also provided herein are methods of producing a plurality of rAAV virion comprising culturing a cell described above in the presence of a first triggering agent and a second triggering agent. In some embodiments, the first triggering agent is doxycycline and the second triggering agent is tamoxifen. In some embodiments, the plurality of rAAV virion have an encapsidation ratio of no less than 0.5, 0.6, 0.7, 0.8, 0.9, 0.95, 0.97, or 0.99 prior to purification. In some embodiments, the plurality of rAAV virion have a F:E ratio of no less than 0.5, 0.6, 0.7, 0.8, 0.9, 0.95, 0.97, or 0.99 prior to purification. In some embodiments, the plurality of rAAV virion have a concentration of greater than 1×10¹¹ or no less than 5×10¹¹, 1×10¹², 5×10¹², 1×10¹³ or 1×10¹⁴ viral genomes per milliliter prior to purification. In some embodiments, the plurality of rAAV virion have an infectivity of no less than 50%, 60%, 70%, 80%, 90%, 95%, or 99% at an MOI of 1×10⁵ vg/target cell or less. In some embodiments, the culturing is in a bioreactor.

Also provided herein are pharmaceutical compositions comprising the rAAV virion produced by the cell or the method described above and a pharmaceutically acceptable carrier. Also provided herein are methods of treating a condition or disorder, the method comprising administering a therapeutically effective amount of the pharmaceutical composition to a patient in need thereof.

### BRIEF DESCRIPTION OF THE DRAWINGS

FIG. 1 depicts the pre-triggered state of an exemplary cell in which a plurality of synthetic nucleic acid constructs have been separately integrated into the nuclear genome. This exemplary cell gives rise to a stable cell line capable of conditionally producing recombinant AAV (rAAV) virions that package a payload (e.g., a therapeutic polynucleotide). The brackets in construct 3 indicate the position of the flanking ITRs.

FIGS. 2A-2C depict an exemplary embodiment of construct 2 from FIG. 1 in greater detail. This construct permits conditional expression of Cre. In the pre-triggered state (top of FIG. 2A) the integrated nucleic acid construct has a Cre coding sequence and adenoviral E2A and E4 helper protein coding sequences collectively under the control of an inducible promoter that becomes active upon the addition of a triggering agent. Other coding elements (activator and a gene that permits mammalian selection (mammalian selection)) are under control of a constitutive promoter (“CMV promoter”). The Cre coding element is positioned between LoxP sites and is additionally fused to estrogen response elements (“ER2”), which allows for control over the localization of Cre in response to estrogen agonists, such as tamoxifen. Upon addition of a triggering agent, Cre is expressed (bottom of FIG. 2A), and upon addition of Tamoxifen, Cre translocates to the nucleus. As shown in FIG. 2B, following translation and translocation of the Cre protein into the cell nucleus, the Cre protein effects excision of its own coding sequence, leaving the integrated construct shown at the bottom of FIG. 2B. Shown in FIG. 2C is an optional insert in construct 2. The optional insert includes a Cre inducible U6 promoter that drives the expression of transcriptionally dead mutants of VA RNA1 (VA RNA). Specifically, the U6 promoter is split into two parts separated by a Lox flanked stuffer sequence. The U6 promoter is inactive because of the presence of the stuffer sequence. Cre mediated excision of the stuffer sequence activates the U6 promoter, which then drives the expression of VA RNA.

FIGS. 3A-3B are schematics depicting details of an exemplary embodiment of construct 1. This construct is designed to permit expression of AAV Rep and Cap proteins from their endogenous promoters after a triggering event. FIG. 3A shows the pre-triggered state of the integrated nucleic acid construct. An intervening spacer (excisable spacer) interrupts the Rep coding sequence. The excisable spacer comprises a first spacer segment, a second spacer segment which is excisable (second “excisable” spacer segment), and a third spacer segment. The second “excisable” spacer segment comprises EGFP flanked by LoxP sites and an upstream 3′ splice site (3′SS). A pre-triggered transcript is shown at the bottom of the figure. This pre-triggered transcript encodes the 5′ portion of AAV rep fused to a fluorescent marker protein, EGFP. The pre-triggered transcript contains a single intron flanked by 5′ splice site (5′SS) and 3′ splice site (3′SS). FIG. 3B shows the conversion of the pre-triggered construct (top schematics) to a post-triggered state (bottom schematics) upon exposure to Cre in the cell nucleus. Cre excises the second “excisable” spacer segment, which includes the EGFP marker coding sequence and the upstream 3′ splice site (3′ SS). When the second “excisable” spacer segment is excised by Cre, the construct allows for expression of functional Rep and Cap transcripts from their respective endogenous promoters.

FIG. 3C shows an alternate embodiment of construct 1. In this embodiment, an intervening spacer interrupts the Rep coding sequence. The intervening spacer comprises a first spacer segment a 5′ splice site (5′SS), a second spacer segment comprising a first recombination site, a first 3′ splice site (3′SS), a detectable protein (e.g., blue fluorescent protein “BFP”) encoding sequence, a second recombination site, a third spacer segment, and second 3′SS. A pre-triggered transcript is shown below the schematic of the pre-triggered construct 1. This pre-triggered transcript encodes the 5′ portion of AAV rep fused to a fluorescent marker protein, BFP. The pre-triggered transcript contains a single intron flanked by 5′ splice site (5′SS) and the first 3′ splice site (3′SS). FIG. 3C also shows the conversion of the pre-triggered construct (top schematic) to a post-triggered state (bottom schematic) upon exposure to Cre in the cell nucleus. Since the first and second recombination sites are present in an opposite orientation, Cre inverts the second spacer segment such that the sequence encoding the BFP and the first 3′SS are no longer operably connected to the p5 and p19 promoters. The sequence encoding BFP and the first 3′SS are present in the transcript in the intron which intron is flanked by the 5′SS and the second 3′SS. When the second spacer segment is inverted by Cre, the construct allows for expression of functional Rep and Cap transcripts from their respective endogenous promoters. Upon inversion of the second spacer segment, BFP is no longer expressed.

FIG. 4 depicts an exemplary embodiment of construct 3. Construct 3 comprises a sequence that encodes a payload (payload polynucleotide). The payload polynucleotide is under control of a constitutive promoter. The brackets indicate the position of the flanking ITRs. As indicated, in various nonlimiting embodiments, the payload is a transgene encoding a protein of interest, a homology element for homology-directed repair (e.g., HDR homology region), or a guide RNA. Also shown is a coding sequence coding for a protein that permits selection in mammalian cells (mammalian selection).

FIG. 5A depicts an exemplary embodiment of a split auxotrophic selection system that permits stable retention of two integrated nucleic acid constructs under a single selective pressure. One construct encodes the N-terminal fragment of mammalian dihydrofolate reductase (DHFR) fused to a leucine zipper peptide (“Nter-DHFR”). This N-terminal fragment is enzymatically nonfunctional. The other construct encodes the C-terminal fragment of DHFR fused to a leucine zipper peptide (“Cter-DHFR”). This C-terminal fragment is enzymatically nonfunctional. When both fragments are concurrently expressed in the cell, a functional DHFR enzyme complex is formed through association of the leucine zipper peptides. Both constructs can be stably retained in the genome of a DHFR null cell by growth in a medium lacking hypoxanthine and thymidine (-HT selection).

FIG. 5B shows an exemplary deployment of this split auxotrophic selection design in the multi-construct system of FIG. 1 in its pre-triggered state. In this example, the split auxotrophic selection elements are deployed in constructs 1 and 3. A separate exemplary antibiotic selection approach, blasticidin resistance, is deployed in construct 2. This results in the ability to stably maintain all three constructs in the mammalian cell line by culturing in medium having a single antibiotic, blasticidin, and lacking both thymidine and hypoxanthine.

FIG. 6 depicts the post-triggered state of all 3 constructs following the addition of tamoxifen and a triggering agent. In the presence of the triggering agent, adenoviral E2A and E4 helper proteins are expressed. AAV rep and cap coding sequences are expressed under control of endogenous promoters. The payload is expressed under control of a constitutive promoter. rAAV virions encapsidating the payload are therefore produced. The three integrated constructs are stably maintained in the nuclear genome with a single antibiotic (Blasticidin) and auxotrophic selection (media lacking both thymidine and hypoxanthine).

FIGS. 7A-7B are light and fluorescence microscopic images. Light microscope images are presented in the left column. Green fluorescence images are presented in the middle column. Red fluorescence images are presented in the right column. Following addition of different amounts of Cre vesicles containing Cre protein and a red fluorescence marker protein, cells were either mock-transfected (“Mock”), transfected with a plasmid having construct 1 (“AAV2 CODE”), or transfected with a control AAV2 plasmid capable of expression Rep and Cap proteins (“AAV2”). Without addition of Cre (FIG. 7A), only the cells transfected with a plasmid having Construct 1 show intense green fluorescence, indicating expression of the Rep-EGFP fusion protein (see bottom of FIG. 3A). FIG. 7B shows decreased EGFP fluorescence in the presence of increasing amounts of Cre, indicating recombination and subsequent removal of EGFP cassette from construct 1.

FIGS. 8A-8B are blots and graphs showing Rep production from post-triggered plasmid construct 1. FIG. 8A shows Western blots illustrating that Cre-mediated excision of the excisable spacer segment induces Rep protein production from post-triggered plasmid construct 1. In addition, the presence of the rabbit beta globin intron does not interfere with Rep protein expression level. FIG. 8B shows a schematic of a Rep/Cap polynucleotide construct, which is cloned into a piggybac vector with a Blasticidin resistance gene (SEQ ID NO: 8). The excisable element interrupting the Rep gene was inserted downstream of the p19 promoter. The GFP levels confirm successful integration of the Rep/Cap construct in cells from STXC0068 cell line (bottom FACS plot) compared to cells from the parental cell line (top FACS plot). Graphs of the cell density (top graph) and viability (bottom graph) data of the STXC0068 cell line illustrate that there were no negative effects from the integrated AAV sequences. The left blot shows the production of Rep proteins and the right blot shows total protein, for the parental cell line, the parental cell line after the addition of Cre, the STXC0068 cell line, and the STXC0068 cell line after the addition of Cre.

FIG. 9A presents a schematic of the PKR pathway interactions.

FIG. 9B shows a diagram VA RNA construct and the sequence of VA RNA1 that shows its double-stranded RNA (dsRNA) structure.

FIGS. 10A-10B depict the plasmid descriptions (FIG. 10A) and testing (FIG. 10B) of these plasmid constructs comprising VA RNA constructs including wildtype VA RNA (pHelper), VA RNA knockout (STXC002), and VA RNA knockout with a compensatory viral protein (infected cell protein 34.5 (ICP34.5); STXC0016) to evaluate the effect of VA RNA on AAV titers.

FIGS. 11A-11B depict the plasmid descriptions (FIG. 11A) and testing of these plasmids (FIG. 11B) comprising VA RNA promoter mutants for the relative VA RNA expression in adherent HEK293 cells.

FIGS. 12A-12D depict the design of an inducible U6 promoter segment containing mutant VA RNA (FIG. 12A), the plasmid descriptions (FIG. 12B) for the control and test plasmids, and the relative VA RNA expression in LV max cells (FIGS. 12C & 12D), illustrating rescue of select mutants with an inducible promoter.

FIGS. 13A-13B are graphs showing titer results using the mutant and inducible VA RNA constructs from FIG. 12B in HEK293T cells (FIG. 13A) and LV Max cells (FIG. 13B).

FIG. 14 shows a plasmid map of STX_C002, which is a helper plasmid without VA RNA expression (VA RNA is deleted).

FIG. 15 shows a plasmid map of STX_C0032, which is a STX_C002helper plasmid backbone containing a WT VA RNA.

FIG. 16 shows a plasmid map of STX_C0033, which is a STX_C002 helper plasmid backbone containing a VA RNA1 B1 mutant (a six-nucleotide segment deleted from the B Box) with the VA RNA in reverse orientation.

FIG. 17 shows a plasmid map of STX_C0036, which is a STX_C002 helper plasmid containing the VA RNA mutations G16A and T45C, with the VA RNA in reverse orientation).

FIG. 18 shows a plasmid map of STX_C0041, which is made by modifying the STX_00033 helper construct containing VA RNA1 B1 mutant (a six-nucleotide segment deleted from the B Box) to contain a U6 inducible promoter construct (as shown in FIG. 12A). The position of the new U6 promoter and Lox sites are shown.

FIG. 19 shows a plasmid map of STX_C0042 which is made by modifying the STX_C0035 helper plasmid containing the VA RNA mutations G16A and T45C to contain a U6 inducible promoter construct (as shown in FIG. 12A). The position of the new U6 promoter and Lox sites are shown.

FIG. 20 shows a plasmid map of STX_C0043 which is made by modifying the STX_C0037 helper plasmid containing the VA RNA mutations G16A and G60A. The position of the new U6 promoter and Lox sites are shown.

FIG. 21 shows a plasmid map of STX_C0037 containing the VA RNA mutations G16A and G60A.

FIG. 22 is a schematic showing the production of an exemplary stable cell line (P2 Producer Cell Line) containing a Rep/Cap construct, an inducible helper construct, and a construct with payload construct and the packaging of the payload (Gene of Interest) into virions. The PI Helper Cell Line is produced from integration of either inducible helper construct #1 or inducible construct #2 into the Serum Free Suspension Adapted 293 cells.

FIG. 23 shows plasmid maps and a graph showing Rep production using inducible bicistronic constructs in a transient transfection system.

FIG. 24 shows schematics of STXC0090 and STXC0110 constructs illustrating helper and Cre induction using the TetOn system. (“E1alpha” refers to “EF1alpha”)

FIG. 25 shows schematics of VA RNA mutant constructs and various promoter and selection options. (“E1alpha” refers to “EF1alpha”)

FIG. 26 shows intracellular staining for expression of FLAG-tagged E2A from cells with stable integration of STXC-0123 (T33, left plot), STXC-0124 (T34, middle plot), or STXC-0125 (T35, right plot) helper constructs, and after either mock induction, no induction, or induction of Cre.

FIG. 27 shows an overview of HEK293 cells with the stably integrated helper plasmid showing no cytotoxic effects and induction of Cre, production of VA RNA and good distribution of E2A expression.

FIG. 28 shows work flows for producing stable cell line pools. The schematic and work flow on the left illustrates integration of STXC0123 to produce the T33 pool, in which STXC0137 and STXC0136 are then integrated, to produce three stable cell line pools (T40, T41, and T42). The schematic and workflow on the right illustrates integration of STXC0133 to produce the T44 pool, in which STXC0137 and STXC0136 are then integrated, to produce three stable cell line pools (T56, T57, and T58). The stable cell line pools are then treated with doxycycline and tamoxifen to produce virions encapsidating STXC650. (“E1alpha” refers to “EF1alpha”)

FIG. 29 shows graphs of the viable cell density (left graph) and viability (right graph) for the T33 pool and T44 pool (FIG. 28), and illustrate that there were no negative effects from the integrated plasmid constructs.

FIG. 30 shows graphs for E2A expression, VA RNA expression, culture density and culture viability for the T33 pool stable cell line, the T44 stable cell line, and the parental cell line (VPC) either not induced or after induction. The left graph is at 24 hours post induction and the right graph is at 48 hours post induction.

FIG. 31 shows graphs of the viable cell density (left graph) and viability (right graph) for the T40, T41, and T42 cell line pools illustrated in FIG. 28.

FIG. 32 shows graphs of the viable cell density (right graph) and viability (left graph) for the T59, T60, and T61 cell line pools, which were produced the same way as the T56, T57, and T58 cell line pools illustrated in FIG. 28.

FIG. 33 shows a graph of capsid production from the T42 pool stable cell line after induction compared to cells produced by transient triple transfection (3×Tfxn) in various cell medias (AAV, Bal, Cyt 2, Cyt9, Fuji 7, Fuji 7-2, HE300, TS1, TS3, or TS5). The left bar for each media type indicates total capsid titer and the right bar for each media type indicates the titer of capsids encapsidating a viral genome (e.g., the payload construct).

FIG. 34 shows the titer of capsids encapsidating a viral genome (e.g., the payload construct) for the T42 stable cell line pool, T59 stable cell line pool, T60 stable cell line pool, and T61 stable cell line pool either with (+) or without (−) induction in HE300 media.

FIG. 35 shows infectivity as indicated by the percentage of GFP+ cells after infecting target cells (CHO Pro-5 cells) with capsids from the T42 pool stable cell line compared to the T61 pool stable cell line in various media. The left bar for each cell line type/media is for a dilution factor of 1 and the right bar for each cell line type/media is for a dilution factor of 4.

FIG. 36 shows a graph of the titer of capsids encapsidating a viral genome (e.g., the payload construct) per cell from the T42 pool stable cell line after induction compared the titer of capsids encapsidating a viral genome (e.g., the payload construct) to per cell from the triple transfected parental cells (VPC) in various cell medias. The left bar for each media type indicates titer of capsids encapsidating a viral genome produced per cell from the T42 pool stable cell line and the right bar for each media type indicates titer of capsids encapsidating a viral genome produced per cell from the triple transfected parental cell line (VPC).

FIG. 37 shows a graph of the dilution adjusted titer of capsids encapsidating a viral genome (e.g., the payload construct) from the T42 pool stable cell line after induction at different seed densities in various cell medias. The 3×Tfxn dashed line indicates the dilution adjusted titer of capsids encapsidating a viral genome (e.g., the payload construct) produced by cells after transient triple transfection (3×Tfxn).

FIG. 38 shows a graph of total capsids in different cell media with mini pool clones selected from the T42 pool stable cell line compared to the T42 pool stable cell line after induction in different cell media.

FIG. 39 shows infectivity as indicated by the percentage of GFP+ cells (encapsidated payload) after infecting target cells (CHO Pro-5 cells) with capsids versus multiplicity of infection (vg/cell) for mini pool clones selected from the T42 pool stable cell line in various cell media from FIG. 38. (Control is a purified capsid produced by cells after transient transfection).

FIG. 40 shows infectivity as indicated by the percentage of GFP+ cells (encapsidated payload) after infecting target cells (CHO Pro-5 cells) with capsids versus multiplicity of infection (vg/cell) for mini pool clones selected from the T42 pool stable cell line in various cell media from FIG. 38. The inset control shows infectivity as indicated by the percentage of GFP+ cells (encapsidated payload) after infecting target cells (CHO Pro-5 cells) with capsids versus multiplicity of infection (vg/cell) for a purified capsid produced by cells after transient transfection.

FIG. 41 shows (top) a workflow for testing reproducibility and stability of clones and (middle; bottom) reproducibility of titer from 1D3 cells (a mini-pool clone from the T42 pool stable cell line) cultured, taken and induced at 12 different time points in a 6-week time period, in which later time points are for cells having an increased number of passages. The bottom graph shows that the clones have increased titer and reproducibility.

FIG. 42 shows titer distributions (as assessed by PCR (top) and ELISA (bottom)) from 1D3 cells (a mini-pool clone from the T42 pool stable cell line) taken and induced at different 12 time points in a 6-week time period, in which later time points are for cells having an increased number of passages.

FIG. 43 shows stability of titer from banked aliquots of frozen 1D3 (a mini-pool clone from the T42 pool stable cell line) that were cultured and banked at different time points over 6 weeks, but then all thawed and induced at the same time. The bottom graph shows that the clone in-vitro cell age did not impact viral titer.

FIG. 44 shows titer (vg/ml) of single clones (from the T42 pool stable cell line) after induction vs. titer (vg/ml) of the T42 pool stable cell line after induction.

FIG. 45 shows titer (vg/ml) (top graph) or packaging efficiency (vg/capsid (%)) (bottom graph) of a subset of top single clones (from the T42 pool stable cell line) after induction vs. a triple transfected control.

FIG. 46 shows cells (Glutamine Synthetase KO cells) that were transfected with a vector system for integration, maintained growth and viability as shown by viable cell density (left) and % viability (right). The vector system comprised the helper construct comprising Glutamine Synthetase as a selectable marker, a Rep/Cap construct comprising a C-term of a split Blasticidin resistance protein as a selectable marker, and a payload construct comprising a N-term of a split Blasticidin resistance protein as a selectable marker. ON:ASE indicates the ratio of transposon to transposase used for integration of the constructs.

FIG. 47 shows that the cells from FIG. 46 that integrated the vector system produced AAV after induction with doxycycline and tamoxifen. The ratios indicate the ratio of transposon to transposase used for initial integration of the constructs. +Gln or −Gln indicates whether the cells were passaged with or without glutamine in the media. Titer was measured by qPCR.

FIGS. 48A-48C show higher yield, higher payload packaging, and enhanced infectivity of AAV produced after induction of single clones from the T42 pool stable cell line. FIG. 48A shows titer of single clones (from the T42 pool stable cell line) after induction compared to the T42 pool stable cell line after induction and compared to titer produced from cells after transient transfection. The top clone shows a greater than 10-fold improvement in viral titer. Titer was measured from AAV5-sc-eGFP lysate pre-purification by capsid ELISA for viral particle. FIG. 48B shows packaging efficiency of virions produced a single clone (from the T42 pool stable cell line) after induction compared to packaging efficiency of virions produced from cells after transient transfection. The single clone showed a 400% higher payload per capsid content prior to purification. Packaging was measure from AAV5-sc-eGFP lysate pre-purification, via ddPCR for viral genome (vg), and by capsid ELISA for viral particle (vp). FIG. 48C shows enhanced infectivity of virions produced after induction from a single clone (from the T42 pool stable cell line) compared to infectivity of virions produced from cells after transient transfection. The single clone showed a 300% improvement in infectivity. Infectivity was measure from GFP expressing CHO cells after transduction with AAV-sc-eGFP lysate pre-purification

FIG. 49A shows a schematic for Rep/Cap amplification by promoter mutagenesis in which a selectable marker, such as glutamine synthetase (GS), is under the control of an attenuated promoter, such as EF1alpha (EF1a) promoter having a TATGTA mutation.

FIG. 49B shows a schematic for Rep/Cap amplification by inhibitor amplification, such as by using methionine sulfoximine (MSX) to inhibit GS.

FIG. 50 shows a schematic of three paths for amplification of Rep/Cap using GS as a selectable marker in a selection system.

FIG. 51. shows a schematic of GS repression/inhibition for the pathways described in FIG. 8 for selection of cells having higher GS expression and/or cells having a higher copy number of integrated constructs comprising GS.

FIG. 52 shows a flowchart of cell line development process for the P3 cells of FIG. 8. T220 cells comprise selected cells that were transfected with a Rep/Cap plasmid comprising a GS selectable marker having an R324C mutation. T221 cells comprise selected cells that were transfected with a Rep/Cap plasmid comprising a GS selectable marker having an R324S mutation. T222 cells comprise selected cells that were transfected with a Rep/Cap plasmid comprising a GS selectable marker having an R3241C mutation. T223 cells comprise selected cells that were transfected with a Rep/Cap plasmid comprising a GS selectable marker (full-length, FL) and selected in media comprising MSX. T224 cells comprise selected cells that were transfected with a Rep/Cap plasmid comprising a GS selectable marker under the control of an EF1alpha promoter having a TATGTA mutation.

FIG. 53 shows viable cell density (left) and percent viability (right) of cells from P3 comprising integrated mutant GS R41C.

FIG. 54 shows viable cell density (left) and percent viability (right of cells from P3 cultured in 50 uM, 100 μM, 250 uM, 500 uM of MSX.

FIG. 55 shows viral particles measured by ELISA (Capsid/mL) (left) or viral genomes measured by qPCR (vg/mL) produced after induction of T222 cells, T223 cells not cultured with MSX, or T223 cells cultured with 1000 uM MSX.

FIG. 56 shows viral genomes measured by qPCR (vg/mL) produced either 5 days or 7 days after induction of T222 cells, T223 cells not cultured with MSX, or T223 cells cultured with 1000 uM MSX, in which induction was in H300 media with or with glutamine or Fuji media with or with glutamine.

FIG. 57 shows viable cell density (VCD) (left) or percent viability (right) 5 days after induction of T222 cells, T223 cells not cultured with MSX, or T223 cells cultured with 1000 uM MSX, in which the cells were induced in H300 media with or with glutamine or Fuji media with or with glutamine.

FIG. 58 shows viable cell density (VCD) (left) or percent viability (right) 7 days after induction of T222 cells, T223 cells not cultured with MSX, or T223 cells cultured with 1000 uM MSX, in which the cells were induced in H300 media with or with glutamine or Fuji media with or with glutamine.

FIG. 59 shows the ratio of the helper construct (Helper), payload construct (Payload), and Rep/Cap construct (Rep) that were integrated into T222 cells, T223 cells cultured in the presence of 50-500 uM MSX, T223 cells cultured in the presence of 50 uM MSX, T223 cells cultured in the presence of 100 uM MSX, T223 cells cultured in the presence of 250 uM MSX, T223 cells cultured in the presence of 500 uM MSX, T223 cells cultured in the presence of 1000 uM MSX, or a control T42 cells using antibiotic selection instead of GS selection for the Rep/Cap construct (left graph) or the ratio of the helper construct (Helper), payload construct (Payload), and Rep/Cap construct (Rep) that were integrated into T222 cells, T223 cells cultured in the presence of 0 uM MSX, or T223 cells cultured in the presence of 1000 uM MSX (right graph).

FIG. 60 shows the ratio of the helper construct (Helper; left bar of each group), payload construct (Payload; middle bar of each group), and Rep/Cap construct (Rep; right bar of each group) that were integrated into T222 cells, T223 cells cultured in the presence of 0 uM MSX, or T223 cells cultured in the presence of 1000 uM MSX (left graph) or the viral genomes as measured by qPCR (vg/mL) on day 5 after induction of T222 cells (left bar), T223 cells cultured in the presence of 0 uM MSX (middle bar), or T223 cells cultured in the presence of 1000 uM MSX (right bar) in Fuji media without glutamine (right graph).

FIG. 61 shows a schematic of a method for producing stable cell lines capable of inducibly generating rAAV encapsidating a progranulin (PGRN) encoding nucleic acid as the payload.

FIG. 62 provides viable cell density and percent viability of stable cell lines transfected with different payload constructs.

FIG. 63 provides titers of rAAV encapsidating a sequence encoding progranulin (vg/ml) and titers of rAAV capsid proteins (vp/ml) produced by stable cell lines transfected with different payload constructs.

FIG. 64 shows titers of viral genome (vg)/ml, viral particles (vp)/ml, and Rep proteins (Rep)/ml produced by clones derived from stable cell line pool T205-RSV Promoter—PGRN: STX1041.

FIG. 65 shows high titer produced after induction of single clones from the stable cell line pool T205. The graph shows the capsid titer (vp/ml) from cell lysate produced after induction of single clones (from the stable cell line pool T205) compared to the stable cell line pool T205 after induction and compared to titer produced from cells after transient transfection. The top clone shows a greater than 10-fold improvement in viral titer over transient transfection titer, indicating that similarly to select single cell clones from the T42 pool stable cell line with an eGFP payload (e.g., Clone D (eGFP)), higher titer can be achieved after induction of select single cell clones from a pool stable cell line (here, from the stable cell line pool T205) having a therapeutically relevant payload (here, a sequence coding for progranulin (PGRN)) compared to transient transfection titer. Titer was measured from AAV5-sc-payload lysate pre-purification by capsid ELISA for viral particle.

FIG. 66 shows progranulin production (PGRN pg/mL) by cells infected at the indicated multiplicity of infection of rAAV virion titers (MOI vg/cell) that were produced after induction of a single cell clone cell line (CL23) selected from the stable cell line pool T205.

### DETAILED DESCRIPTION

To solve the problems presented by transient transfection approaches to rAAV production while addressing the toxicity of AAV Rep protein when constitutively expressed, disclosed herein are polynucleotide constructs and cell lines stably integrated with said polynucleotide constructs (referred to herein as “stable cell lines”) that enable conditional (also referred to herein as “inducible”) production of recombinant AAV (rAAV) virions. In some embodiments, the compositions and methods of use thereof as disclosed herein provide rAAV virions that encapsidate a desired expressible payload, such as an expressible therapeutic payload. Further provided herein is a stable mammalian cell line, wherein the cells are capable of conditionally producing recombinant AAV (rAAV) virions within which are packaged an expressible payload; and wherein a population of virions produced by the stable cell are more homogenous than a population of virions produced by an otherwise comparable cell producing rAAV virions upon transient transfection.

Further provided herein is a stable mammalian cell line, wherein the cells are capable of conditionally producing recombinant AAV (rAAV) virions within which are packaged an expressible payload; and production of virions is inducible upon addition of a triggering agent.

Further provided herein is a stable mammalian cell line, wherein the cells are capable of conditionally producing recombinant AAV (rAAV) virions within which are packaged an expressible payload; and production of virions is not conditioned on the presence of a plasmid within the cell.

### Definitions

Unless defined otherwise, all technical and scientific terms used herein have the meaning commonly understood by one of ordinary skill in the art to which the invention pertains.

“Recombinant”, as applied to an AAV virion, means that the rAAV virion (synonymously, rAAV virus particle) is the product of one or more procedures that result in an AAV particle construct that is distinct from an AAV virion in nature.

In some aspects, the disclosure provides transfected host cells. The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell which has been transfected. Thus, a “host cell” may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation. A “host cell” as used herein may refer to any mammalian cell which is capable of functioning as an adenovirus packaging cell, i.e., expresses any adenovirus proteins essential to the production of AAV, such as HEK 293 cells and their derivatives (HEK293T cells, HEK293F cells), HeLa, A549, Vero, CHO cells or CHO-derived cells, and other packaging cells.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

The term “cell culture,” refers to cells grown adherent or in suspension, bioreactors, roller bottles, hyperstacks, microspheres, macrospheres, flasks and the like, as well as the components of the supernatant or suspension itself, including but not limited to rAAV particles, cells, cell debris, cellular contaminants, colloidal particles, biomolecules, host cell proteins, nucleic acids, and lipids, and flocculants. Large scale approaches, such as bioreactors, including suspension cultures and adherent cells growing attached to microcarriers or macrocarriers in stirred bioreactors, are also encompassed by the term “cell culture.” Cell culture procedures for both large and small-scale production of proteins are encompassed by the present disclosure.

As used herein, the term “intermediate cell line” refers to a cell line that contains the AAV rep and cap components integrated into the host cell genome or a cell line that contains the adenoviral helper functions integrated into the host cell genome.

As used herein, the term “packaging cell line” refers to a cell line that contains the AAV rep and cap components and the adenoviral helper functions integrated into the host cell genome. A payload construct must be added to the packaging cell line to generate rAAV virions.

As used herein, the term “production cell line” refers to a cell line that contains the AAV rep and cap components, the adenoviral helper functions, and a payload construct. The rep and cap components and the adenoviral helper functions are integrated into the host cell genome. The payload construct can be stably integrated into the host cell genome or transiently transfected. rAAV virions can be generated from the production cell line upon the introduction of one or more triggering agents in the absence of any plasmid or transfection agent.

As used herein, the term “downstream purification” refers to the process of separating rAAV virions from cellular and other impurities. Downstream purification processes include chromatography-based purification processes, such as ion exchange (IEX) chromatography and affinity chromatography.

The term “prepurification yield” refers to the rAAV yield prior to the downstream purification processes. The term “postpurification yield” refers to the rAAV yield after the downstream purification processes. rAAV yield can be measured as viral genome (vg)/L.

The encapsidation ratio of a population of rAAV virions can be measured as the ratio of rAAV viral particle (VP) to viral genome (VG). The rAAV viral particle includes empty capsids, partially full capsids (e.g., comprising a partial viral genome), and full capsids (e.g., comprising a full viral genome).

The F:E ratio of a population of rAAV virions can be measured as the ratio of rAAV full capsids to empty capsids. The rAAV full capsid particle includes partially full capsids (e.g., comprising a partial viral genome) and full capsids (e.g., comprising a full viral genome). The empty capsids lack a viral genome.

The potency or infectivity of a population of rAAV virions can be measured as the percentage of target cells infected by the rAAV virions at a multiplicity of infection (MOI; viral genomes/target cell). Exemplary MOI values are 1×101, 1×102, 2×103, 5×104, or 1×105 vg/target cell. An MOI can be a value chosen from the range of 1×10¹ to 1×10⁵ vg/target cell.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. The use of the term “vector” throughout this specification refers to either plasmid or viral vectors, which permit the desired components to be transferred to the host cell via transfection or infection. For example, an adeno-associated viral (AAV) vector is a plasmid comprising a recombinant AAV genome. In some embodiments, useful vectors are contemplated to be those vectors in which the nucleic acid segment to be transcribed is positioned under the transcriptional control of a promoter.

The phrases “operatively positioned,” “operatively linked,” “under control” or “under transcriptional control” means that the promoter is in the correct location and orientation in relation to the nucleic acid to control RNA polymerase initiation and expression of the gene.

The term “expression vector or construct” or “synthetic construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or functional RNA (e.g., guide RNA) from a transcribed gene.

The term “auxotrophic” or “auxotrophic selection marker” as used herein refers to the usage of a medium lacking a supplement, such as a medium lacking an essential nutrient such as the purine precursors hypoxanthine and thymidine (HT), or the like, for selection of a functional enzyme which allows for growth in the medium lacking the essential nutrient, e.g., a functional dihydrofolate reductase or the like.

The term cytostatic as used herein refers to a cellular component or agent/element or condition that inhibits cell growth. Cytostasis is the inhibition of cell growth and multiplication.

The term cytotoxic as used herein refers to quality of being toxic to cells. For instance, cells exposed to a cytotoxic agent or condition may undergo necrosis, in which they lose membrane integrity and die rapidly as a result of cell lysis. Cells exposed to a cytotoxic agent can also stop actively growing and dividing (a decrease in cell viability), or the cells can activate a genetic program of controlled cell death (apoptosis).

As used herein, a “monoclonal cell line” or “monoclonality” is used to describe cells produced from a single ancestral cell by repeated cellular replication. Thus, “monoclonal cells” can be said to form a single clone.

The terms “tetracycline” is used generically herein to refer to all antibiotics that are structurally and functionally related to tetracycline, including tetracycline, doxycycline, demeclocycline, minocycline, sarecycline, oxytetracycline, omadacycline, or eravacycline.

The terms “constitutive” or “constitutive expression” are used interchangeably herein. They refer to genes that are transcribed in an ongoing manner. In some embodiments, the terms refer to the expression of a therapeutic payload or a nucleic acid sequence that is not conditioned on addition of an expression triggering agent to the cell culture medium.

The term “expressible therapeutic polynucleotide or “expressible polynucleotide encoding a payload” or “payload polynucleotide” or “payload” refers to a polynucleotide that is encoded in an AAV genome vector (“AAV genome vector”) flanked by AAV inverted terminal repeats (ITRs). A payload disclosed herein may be a therapeutic payload. A payload may include any one or combination of the following: a transgene, a tRNA suppressor, a guide RNA, or any other target binding/modifying oligonucleotide or derivative thereof, or payloads may include immunogens for vaccines, and elements for any gene editing machinery (DNA or RNA editing). Payloads can also include those that deliver a transgene encoding antibody chains or fragments that are amenable to viral vector-mediated expression (also referred to as “vectored or vectorized antibody” for gene delivery). See, e.g., Curr Opin HIV AIDS. 2015 May; 10 (3): 190-197, describing vectored antibody gene delivery for the prevention or treatment of HIV infection. See also, U.S. Pat. No. 10,780,182, which describes AAV delivery of trastuzumab (Herceptin) for treatment of HER2+brain metastases. A payload disclosed herein may not be a therapeutic payload (e.g., a coding for a detectable marker such as GFP).

In particular, in some instances the payload polynucleotide refers to a polynucleotide that can be a homology element for homology-directed repair, or a guide RNA to be delivered for a variety of purposes. In some embodiments, the transgene refers to a nucleic acid sequence coded for expression of guide RNA for ADAR editing or ADAT editing. In some embodiments, the transgene refers to a transgene packaged for gene therapy. In some embodiments, the transgene refers to synthetic constructs packaged for vaccines.

### Exemplary System Overview

The stable mammalian cell line relies on stable integration and maintenance of a plurality of synthetic nucleic acid constructs within the nuclear genome of the cell. One of these constructs permits inducible expression of a hormone-activated excising element. The excising element can be a recombinase. The recombinase can be a site-specific recombinase. The site-specific recombinase can be a Cre polypeptide or a flippase. Triggering of Cre expression leads to genomic rearrangements, which in turn lead to expression of adenovirus helper proteins, expression of AAV Rep and Cap proteins, and production of rAAV, optionally, encapsidating a therapeutic payload (e.g., transgene, a tRNA suppressor, a guide RNA, or other oligonucleotide). These elements can be in one or more constructs, in any combination that is capable of conditionally producing AAV virion.

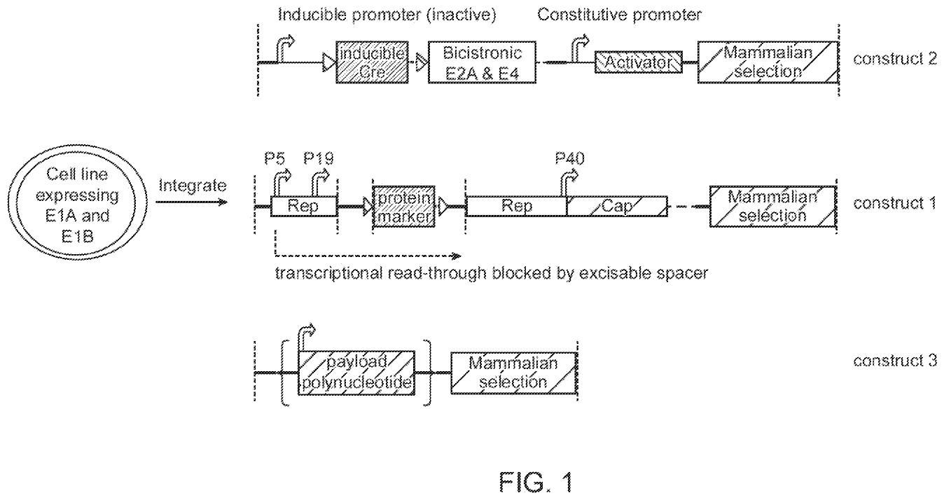

FIG. 1 depicts the pre-triggered state of an exemplary embodiment. In the embodiment shown, three synthetic nucleic acid constructs are separately integrated into the nuclear genome of a cell line that expresses adenovirus E1A and E1B, such as HEK 293 cells. In the pre-triggered state, transcriptional read-through of rep on construct 1 is blocked by an intervening spacer. The payload polynucleotide on construct 3 is flanked by AAV ITRs, represented by the brackets.

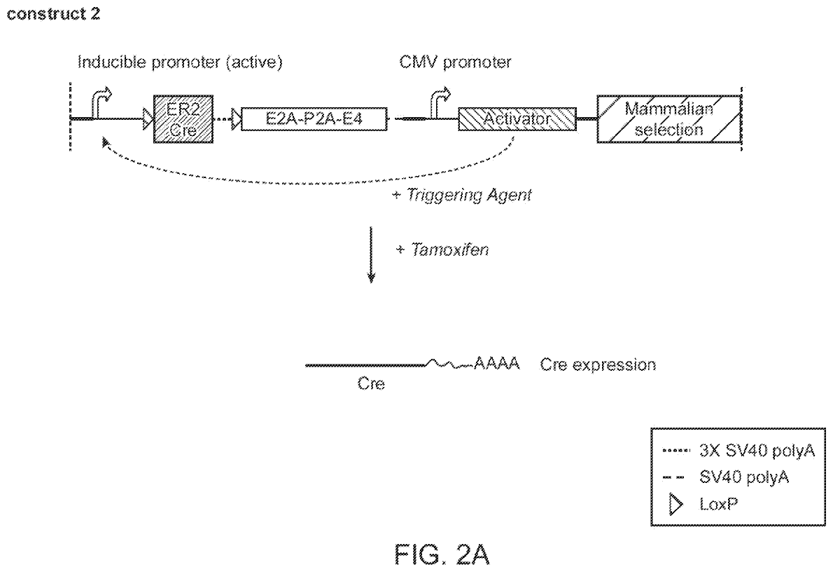

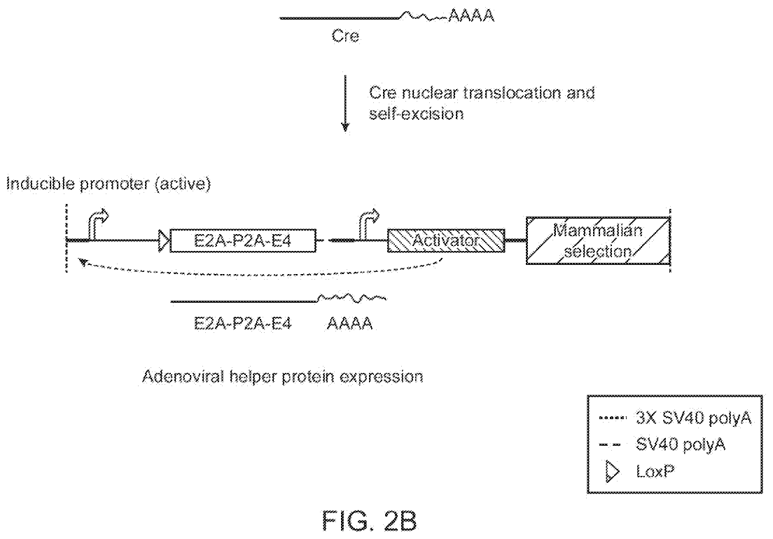

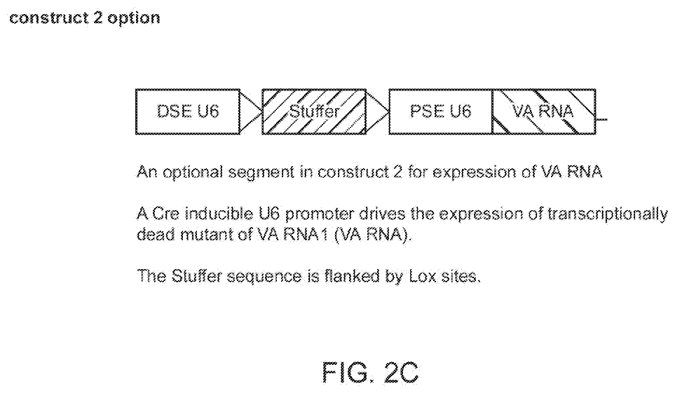

An exemplary construct 2 is shown in greater detail in FIGS. 2A-2C. This construct permits conditional expression of Cre. In some embodiments, the construct 2 comprises a P2A sequence positioned between an E2A sequence and an E4 sequence. In some embodiments, the construct 2 comprises an internal ribosomal entry site (IRES) sequence positioned between an E2A sequence and an E4 sequence. An IRES can comprise at least 80%, 85%, 90%, 95%, 98%, 99% or 100% sequence identity to SEQ ID N: 135. In some embodiments, the inducible promoter system of construct 2 is a Tet On inducible promoter system. In some embodiments, the inducible promoter system of construct 2 is a Tet Off inducible promoter system. In some embodiments, the inducible promoter system of construct 2 is a cumate inducible promoter system.

In the pre-triggered state (top of FIG. 2A), the Cre coding sequence is under the control of an inducible promoter. For example, the inducible promoter is a Tet-inducible promoter. In the absence of a triggering agent, inducible promoter is not active. For example, a triggering agent for Tet-inducible promoter is a tetracycline. In the absence of a tetracycline, such as doxycycline (“Dox”), Tet activator protein (TetOn3G) cannot bind and activate the basal Tet On promoter. In addition, the localization of Cre is under control of estrogen response elements (“ER2”) that require binding of an estrogen agonist or selective modulator, such as tamoxifen, for the translocation from the cytoplasm to the nucleus. This approach limits pre-triggering Cre expression with consequent promiscuous recombination events and toxicity. The ER2 Cre element also comprises a strong 3′ polyadenylation signal, which prevents basal expression of the downstream adenoviral helper genes, E2A and E4. In some embodiments, the Cre is split into two fragments, that can be fused in the presence of a chemical agent, such as rapamycin. In some embodiments, the Cre is a light inducible Cre.

When the triggering agent (e.g., Dox) and tamoxifen are added to the culture medium, TetOn3G binds the Tet responsive basal promoter and estrogen response elements are activated, triggering Cre expression (bottom of FIG. 2A). Following translation and then translocation of the Cre protein into the cell nucleus, Cre excises its own coding sequence from construct 2, leaving the integrated construct shown at the bottom of FIG. 2B. Elimination of the upstream poly-adenylation site allows expression of E2A and E4 helper proteins, maintained by the presence of doxycycline. Similarly, for the optional additional insert shown in FIG. 2C, VA-RNA is expressed by Cre mediated excision of the stuffer sequence, which activates the U6 promoter which then drives the expression of VA RNA.

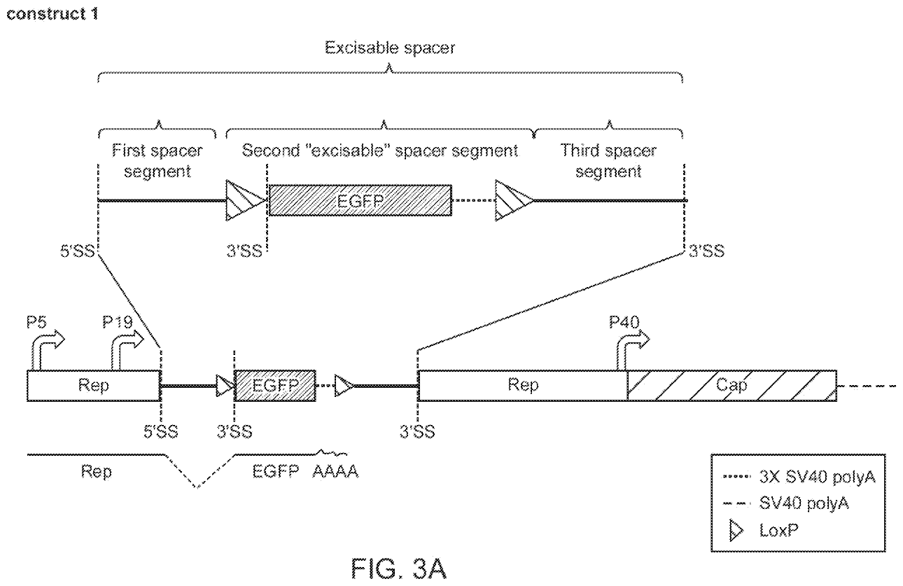

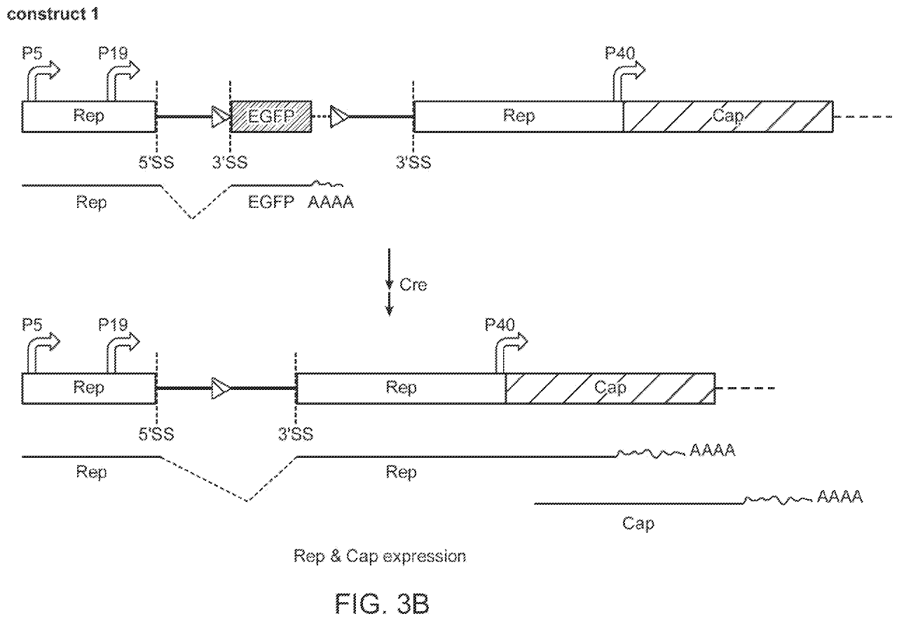

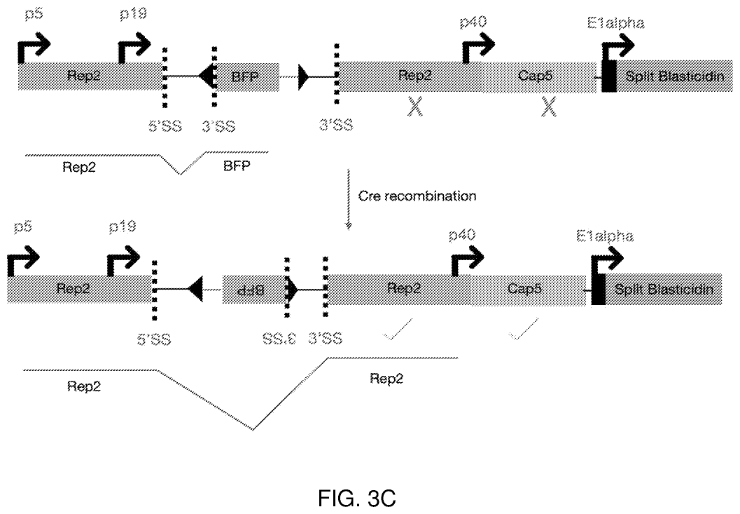

FIGS. 3A-3B schematically depict details of an exemplary embodiment of construct 1. This construct is designed to prevent expression of AAV Rep prior to a triggering event, yet permit expression of AAV Rep and Cap proteins from their endogenous promoters after a triggering event.

FIG. 3A shows the pre-triggered state of integrated nucleic acid construct 1. An excisable spacer interrupts the rep coding sequence, blocking transcriptional read-through of the full-length rep coding sequence. A pre-triggered transcript is shown at the bottom of the figure. This pre-triggered transcript encodes the 5′ portion of AAV Rep fused to a fluorescent marker protein, EGFP. The transcript contains a single intron flanked by 5′ and 3′ splice sites. Routine splicing produces a transcript that encodes a fusion protein that includes the N-terminal portion of rep fused to an enhanced green fluorescent protein (EGFP). The fusion protein lacks the toxicity of full-length Rep protein, and presence of pre-triggered construct 1 in the cell genome can be detected by EGFP fluorescence for quality control. In some embodiments, the EGFP fluorescence is used to select for cells that have integrated nucleic acid construct 1, which then form a stable cell pool. A stable cell pool with the integrated nucleic acid construct 1 can therefore be produced from selecting for cells expressing EGFP.

As shown at the top of FIG. 3A, the excisable spacer comprises a first spacer segment, a second spacer segment, and a third spacer segment. The excisable spacer is inserted at an insertion site between the p19 internal promoter and the p40 internal promoter of the AAV Rep coding sequence. In some embodiments, the insertion site is CAG-G, CAG-A, AAG-G, or AAG-A, wherein the dash (−) indicates the point of insertion of the excisable spacer.

FIG. 3B shows the conversion of the pre-triggered construct (above) to a post-triggered state (below) upon exposure to Cre within the cell nucleus. Cre excises the second spacer segment, which includes the EGFP marker coding sequence and the upstream 3′ splice site. As rearranged, the construct now allows expression of functional Rep and Cap transcripts from their respective endogenous promoters, as shown at the bottom of FIG. 3B. Loss of EGFP expression indicates successful Cre-mediated genomic recombination.

FIG. 4 depicts an exemplary embodiment of construct 3, which is an exemplary payload construct. Construct 3 comprises a sequence that encodes a payload. This sequence element is under control of a constitutive promoter. The payload can be any payload for which rAAV is an appropriate vehicle, including a transgene encoding a protein of interest, a homology element for homology-directed repair, or a guide RNA. The payload is flanked by AAV ITRs, represented by the brackets.

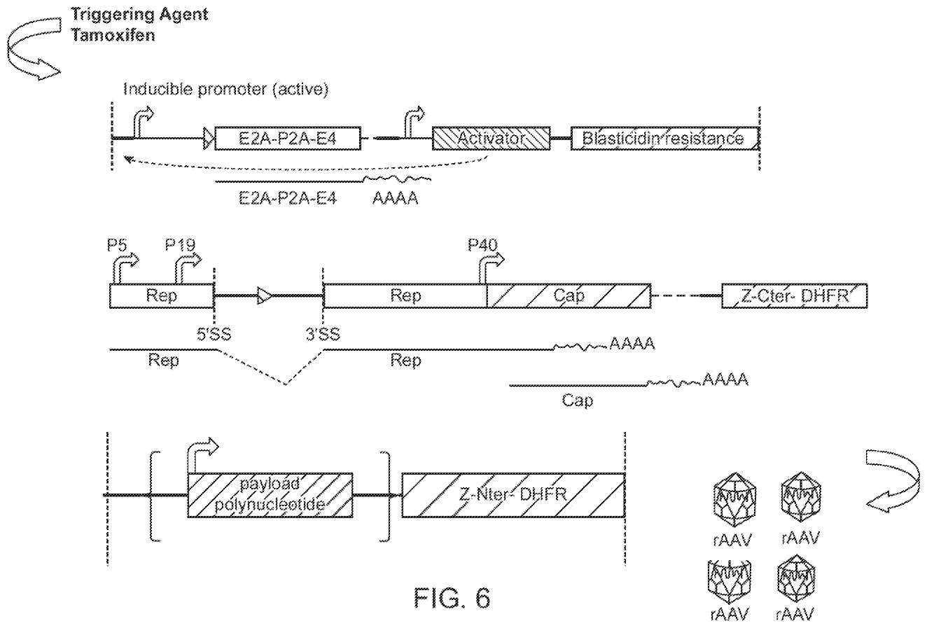

FIG. 6 depicts the post-triggered state of all 3 constructs following the addition of tamoxifen and doxycycline to the cell medium. Adenoviral E2A and E4 helper proteins are expressed from integrated construct 2 under control of the inducible promoter (e.g., a Tet-On promoter activated in the presence of Dox). AAV rep and cap coding sequences are expressed from construct 1 under control of endogenous promoters. The payload is expressed under control of a constitutive promoter. rAAV virions that encapsidate the payload are therefore produced.

This approach provides numerous benefits over current AAV systems for delivery of payloads.

Maintaining constructs stably in the cellular genome requires selective pressure. To reduce the number of selective agents (and in particular, antibiotics) required to stably maintain three integrated constructs within the cell line genome, we have designed an approach that stably maintains all 3 constructs in the nuclear genome with a single antibiotic selection, plus a single auxotrophic selection. In some embodiments, the cell line stably maintains all 3 constructs in the nuclear genome with no antibiotic selection. In some embodiments, the cell line stably maintains all 3 constructs in the nuclear genome utilizing auxotrophic protein selection. In some embodiments, the auxotrophic protein selection is with two auxotrophic protein selections.

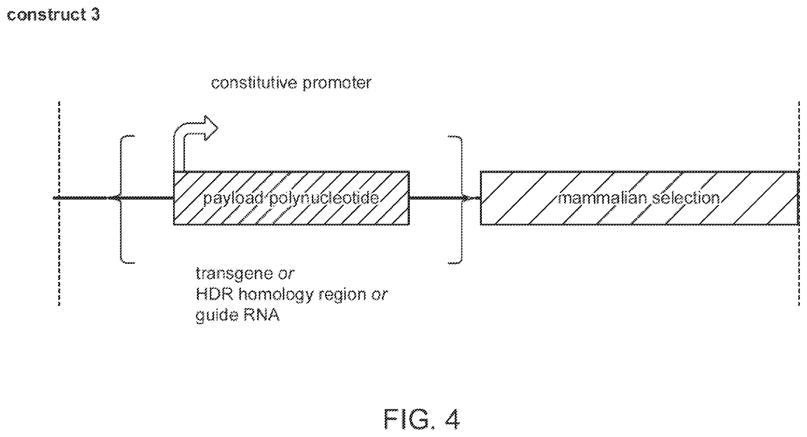

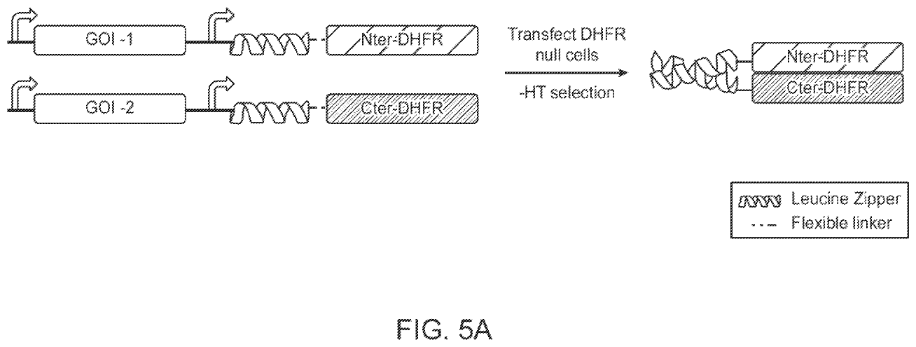

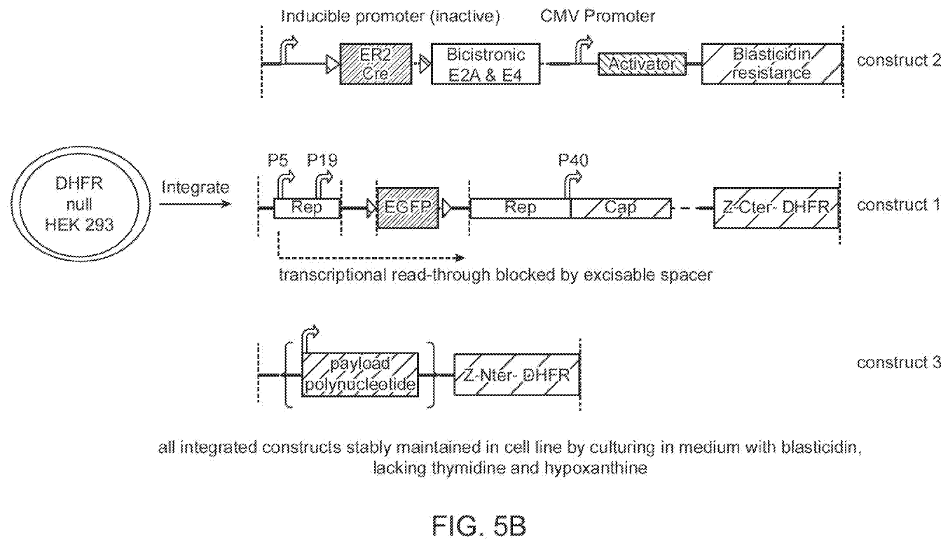

FIG. 5A depicts a split auxotrophic selection system that permits stable retention of two integrated nucleic acid constructs under a single selective pressure. One construct encodes the N-terminal fragment of mammalian dihydrofolate reductase (DHFR) fused to a leucine zipper peptide (“Nter-DHFR”). This N-terminal fragment is enzymatically nonfunctional. The other construct encodes the C-terminal fragment of DHFR fused to a leucine zipper peptide (“Cter-DHFR”). This C-terminal fragment is enzymatically nonfunctional. When both fragments are concurrently expressed in the cell, a functional DHFR enzyme complex is formed through association of the leucine zipper peptides. Both constructs can be stably retained in the genome of a DHFR null cell by growth in a medium lacking hypoxanthine and thymidine.

FIG. 5B shows an exemplary deployment of this split auxotrophic selection design in the multi-construct system of FIG. 1 in its pre-triggered state. In this example, the split auxotrophic selection elements are deployed on constructs 1 and 3. A separate exemplary antibiotic selection approach, blasticidin resistance, is deployed on construct 2. This results in the ability to stably maintain all three constructs in the mammalian cell line using a single antibiotic, culturing in medium with blasticidin, lacking thymidine and hypoxanthine.

In some embodiments, the split auxotrophic selection system is a split intervening proteins (inteins) system that permits stable retention of two integrated nucleic acid constructs under a single selective pressure. Inteins auto catalyze a protein splicing reaction that results in excision of the intein and joining of the flanking amino acids (extein sequences) via a peptide bond. Inteins exist in nature as a single domain within a host protein or, less frequently, in a split form. For split inteins, the two separate polypeptide fragments of the intein must associate in order for protein trans-splicing to occur to excise the intein. Split intein systems are described in: Cheriyan et al, J. Biol. Chem 288:6202-6211 (2013); Stevens et al, PNAS 114:8538-8543 (2017); Jillette et al., Nat Comm 10:4968 (2019); US 2020/0087388 A1; and US 2020/0263197 A1. In some embodiments, the split auxotrophic selection system described herein comprises a construct encoding an N-terminal fragment of an auxotrophic protein fused to an N-terminal intein of the split intein and a construct encoding the C-terminal fragment of the auxotrophic protein fused to a C-terminal intein of the split intein. This N-terminal fragment is enzymatically nonfunctional and this C-terminal fragment is enzymatically nonfunctional. When both fragments are concurrently expressed in the cell, the split inteins can catalyze the joining of the N-terminal fragment of the auxotrophic protein and a C-terminal fragment of the auxotrophic protein to form a functional enzyme, such as any one of the enzymes disclosed herein (e.g., PAH, GS, TYMS, DHFR). In some embodiments, both constructs can be stably retained in the genome of a cell by growth in a medium lacking the product produced by the enzyme. In some embodiments, the split auxotrophic selection elements (e.g., a construct encoding an N-terminal fragment of an auxotrophic protein fused to an N-terminal intein of the split intein and a construct encoding the C-terminal fragment of the auxotrophic protein fused to a C-terminal intein of the split intein) deployed on, for example, constructs 1 and 3, are part of a split intein system. A separate exemplary auxotrophic selection approach, e.g., a full length auxotrophic protein, can be deployed on construct 2. In some embodiments, a construct encoding an N-terminal fragment of an auxotrophic protein fused to an N-terminal intein of the split intein or a construct encoding the C-terminal fragment of the auxotrophic protein fused to a C-terminal intein of the split intein further encodes a helper enzyme, wherein expression of the helper enzyme facilitates growth of the host cell in conjunction with the functional enzyme upon application of the single selective pressure.

Following triggering and Cre-mediated genomic rearrangement, the selection elements remain unchanged, allowing continued maintenance of the three post-triggering integrated constructs using a single antibiotic in medium lacking hypoxanthine and thymidine.

Viral proteins needed for AAV virion formation are inhibited by host cell mechanisms. Inhibition of these host cell mechanisms to maximize AAV viral titers in the stable cell lines described herein include, but are not limited to: knocking out PKR (PKR KO) (pathway is responsible for inhibition of viral proteins) in the starting cell line (P0), introducing a mutant EIF2alpha (in the PKR pathway) in the starting cell line (P0), and/or manipulating or modulating virus-associated (VA) RNAs (VA RNAs, an inhibitor of PKR). Virus-associated (VA) RNAs from adenovirus act as small-interference RNAs and are transcribed from the vector genome. These VA RNAs can trigger the innate immune response. Moreover, VA RNAs are processed to functional viral miRNAs and disturb the expression of numerous cellular genes. Therefore, VA-deleted adenoviral vector production constructs (AdVs) lacking VA RNA genes, or having modified VA RNA, would be advantageous. However, VA-deleted AdVs do not produce commercially sufficient quantities of AAV titers (e.g., resulting in fewer and poor-quality virions). Conversely, overexpressing VA RNA also results in a low titer of AAV production that would not be commercially feasible for scale-up. Thus, developing conditional VA RNA constructs, and combining any of those optimized constructs with the conditional helper constructs described herein, will provide commercially relevant, high-quality virions from the AAV production systems as described herein. All three of these strategies can be done in any combination.

VA RNA is also an inhibitor of PKR, which is involved in a pathway responsible for inhibiting AAV viral protein synthesis. In particular, PKR phosphorylates EIF2alpha, which results in inhibition of viral protein synthesis. FIG. 4A shows a schematic of VA RNA inhibition of PKR and the PKR pathway is shown below at left. The structure of VA RNA, which is a double stranded RNA (dsRNA) is shown in FIG. 4B.

While the limited interactions between VA RNA, PKR, and EIF2alpha are understood, PKR is a major kinase that may self-phosphorylate and EIF2alpha may be phosphorylated by other kinases. As such, three strategies (PKR KO, EIF2alpha mutation, manipulation of VA RNA) are being developed for use in any combination in the AAV production systems described herein.

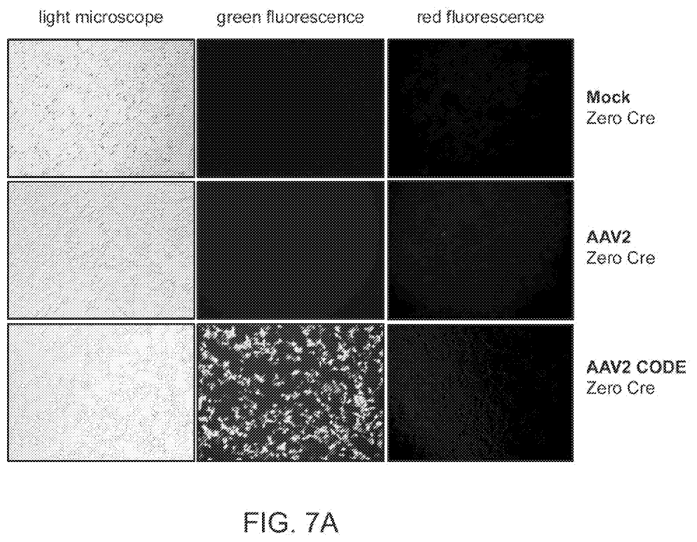

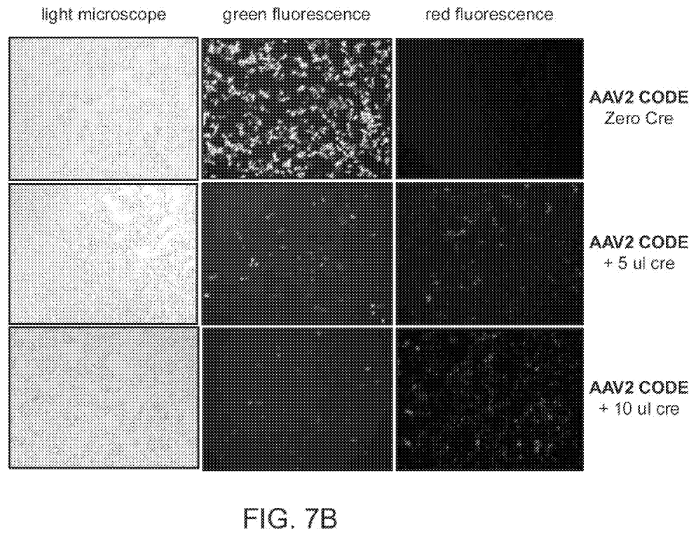

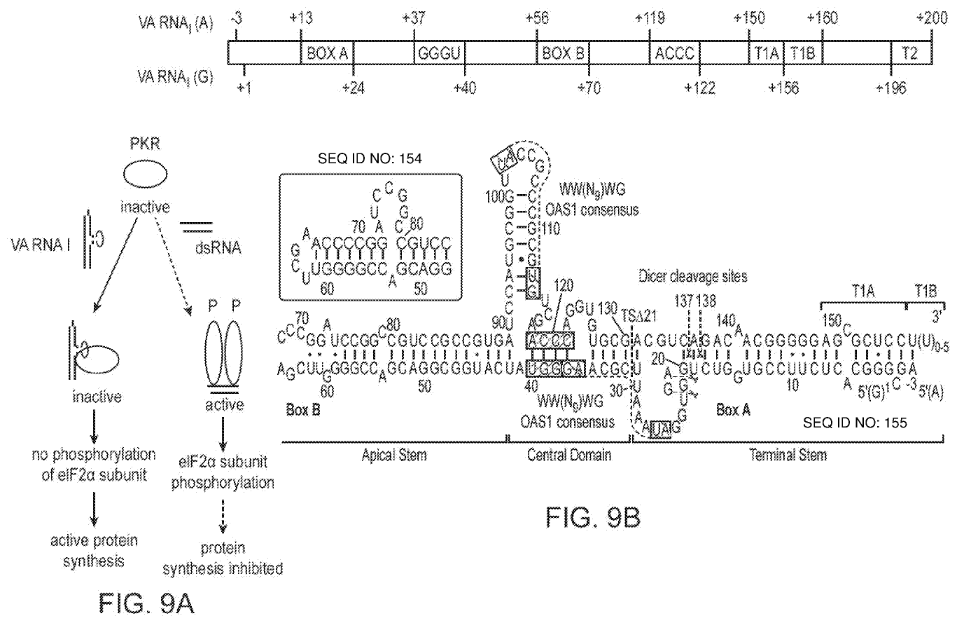

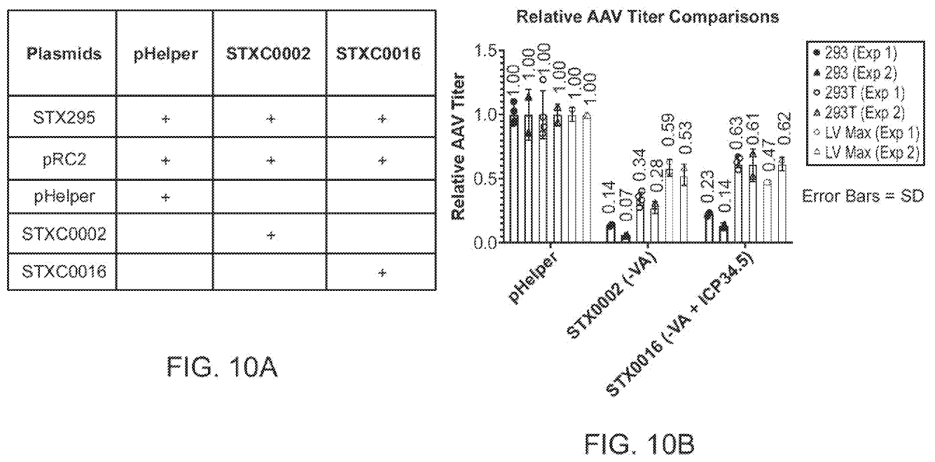

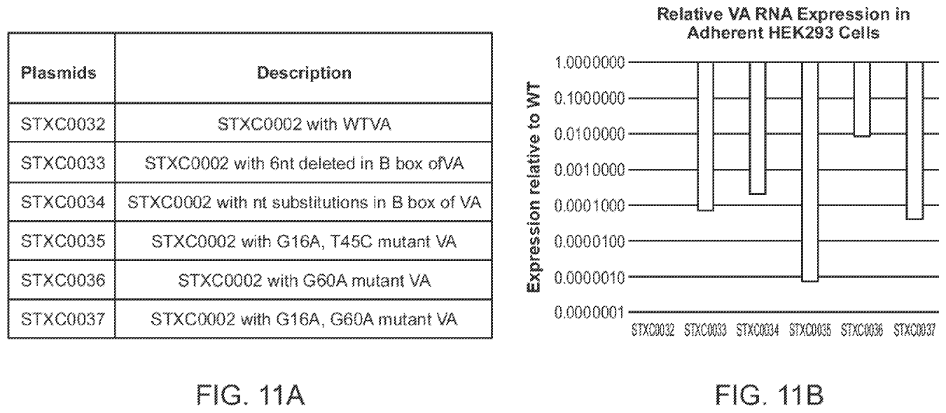

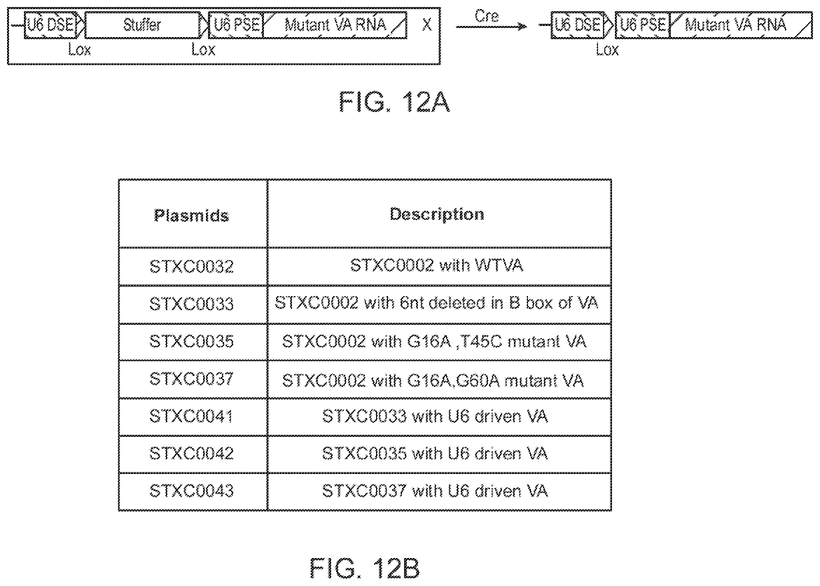

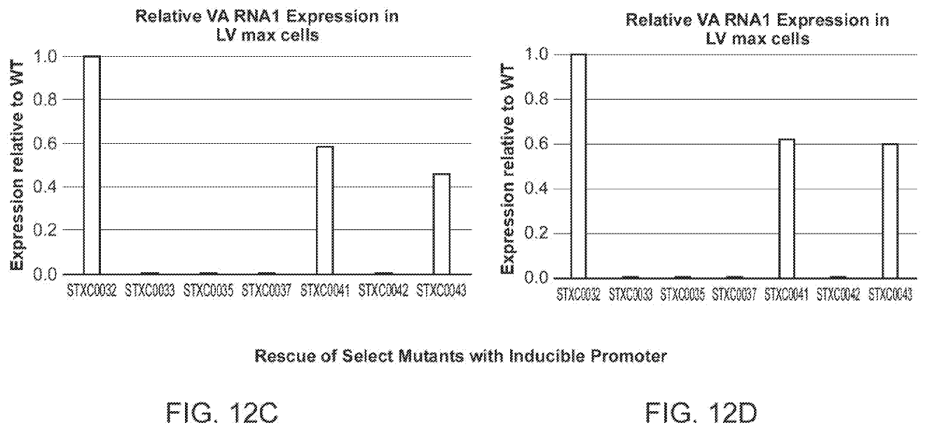

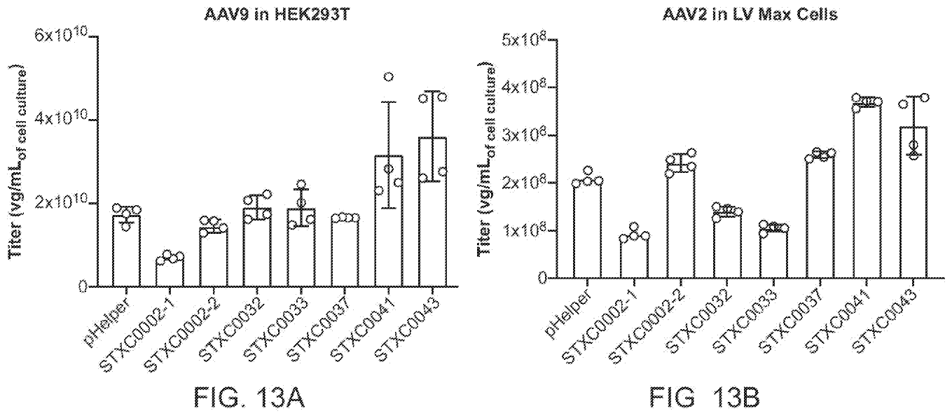

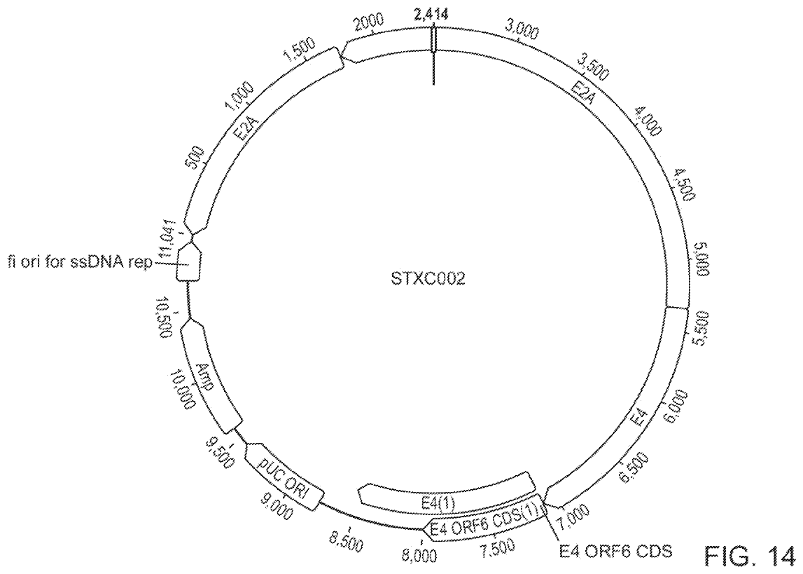

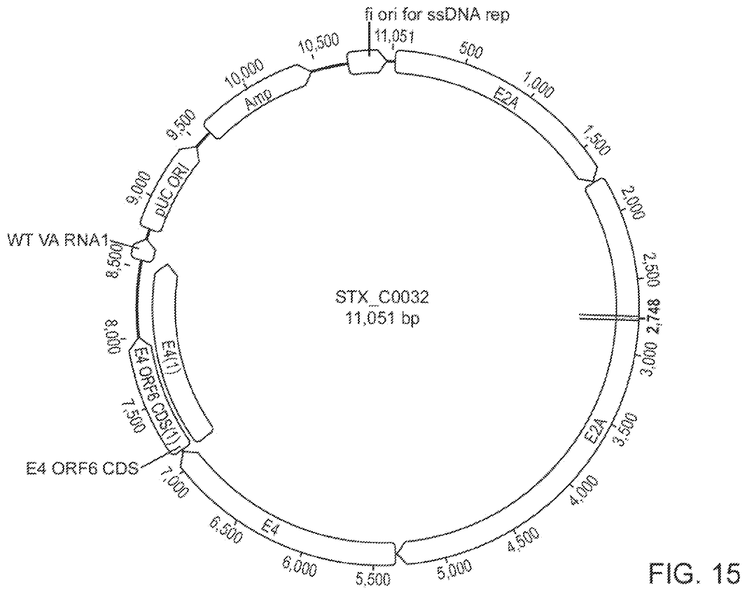

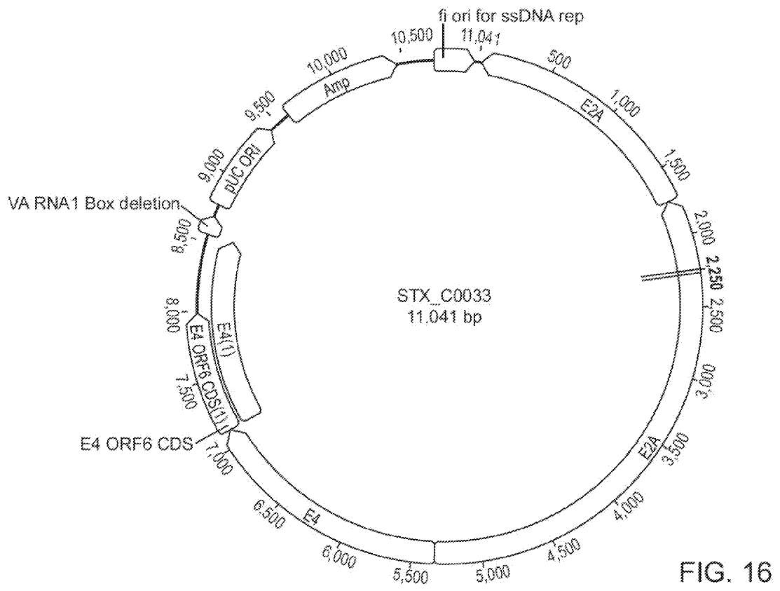

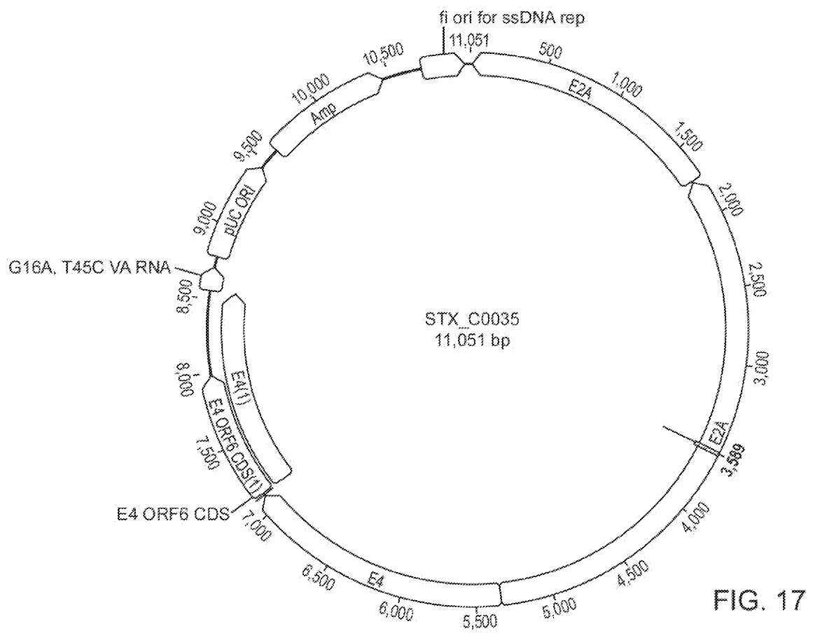

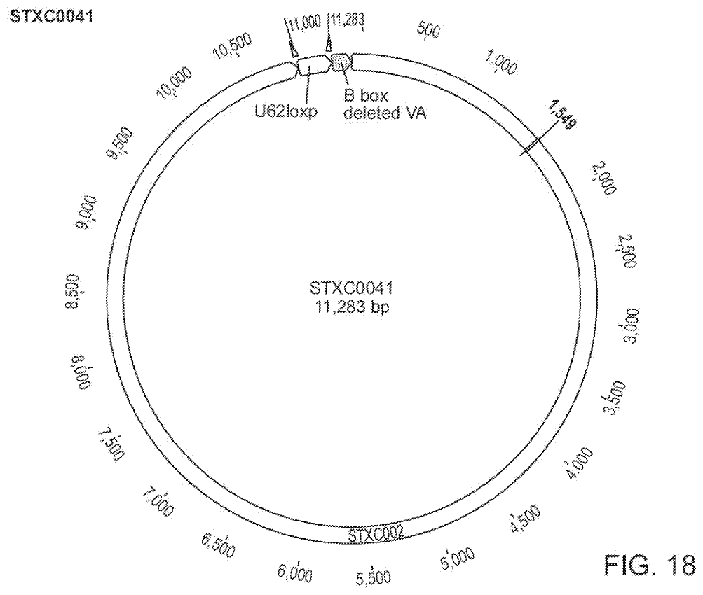

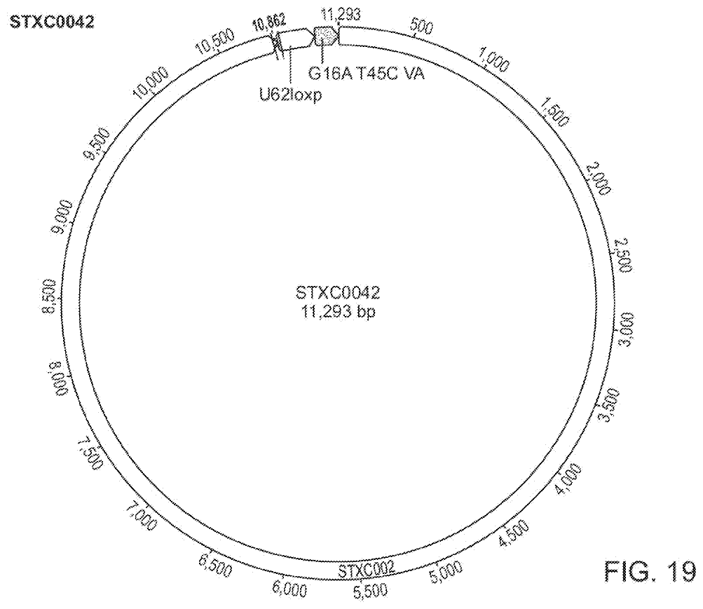

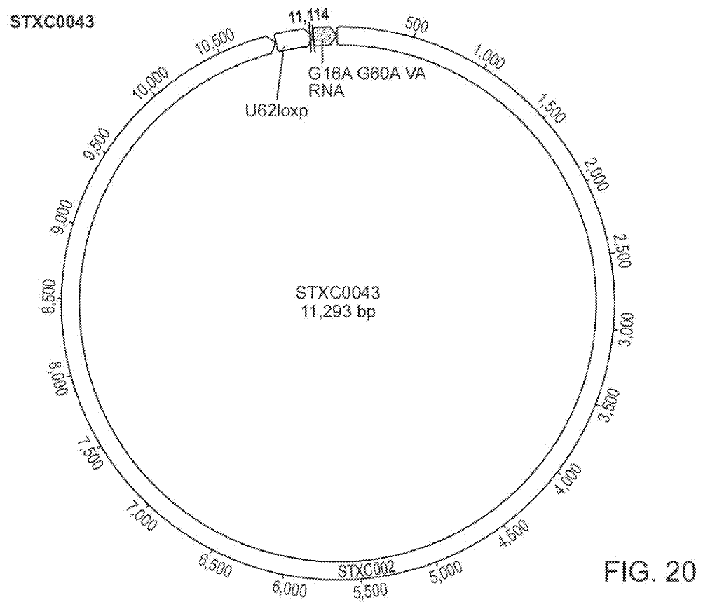

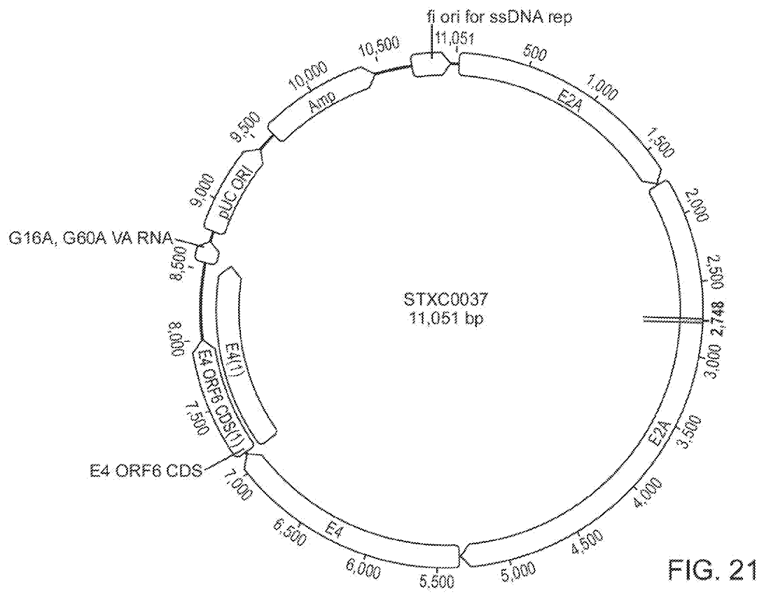

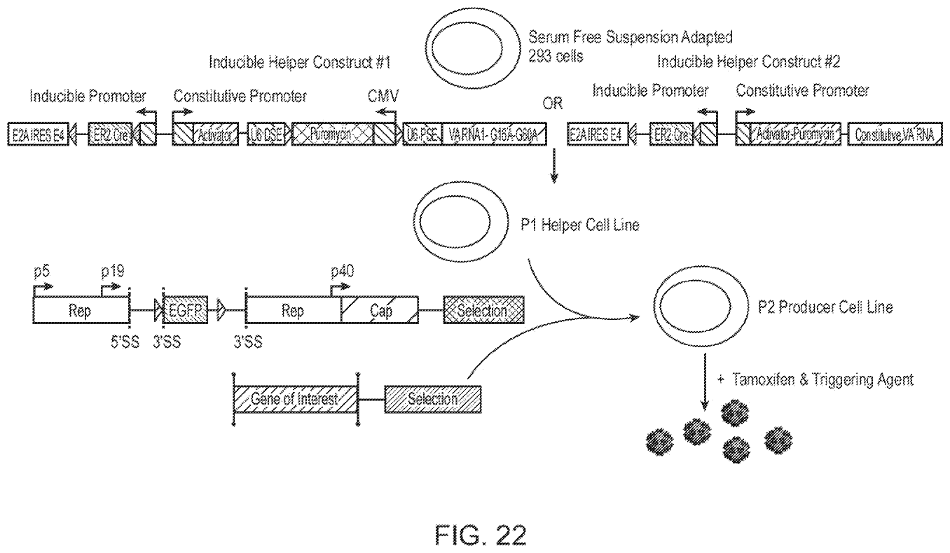

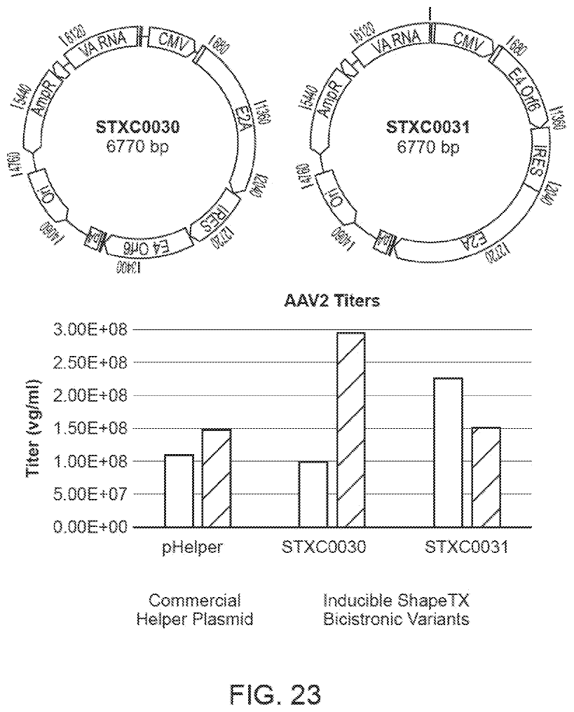

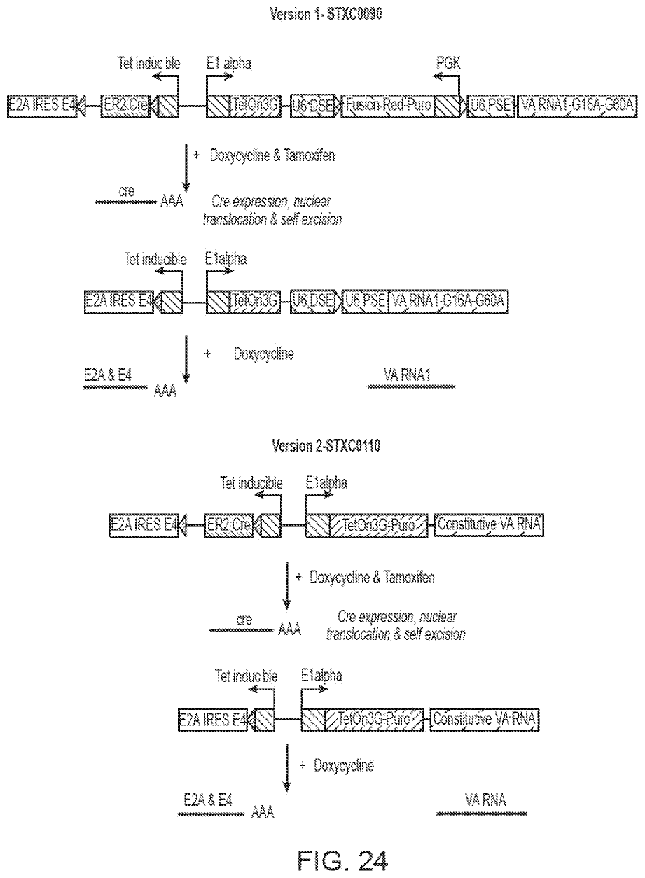

Thus, an option for overcoming the general antiviral effects of mammalian cell production of AAV virions is to modify expression of VA RNA. Therefore, VA-deleted adenoviral vector production constructs (AdVs) lacking VA RNA genes, or having modified VA RNA, have been designed and are described herein in FIG. 2C and FIGS. 4-15. It is noted that VA-deleted AdVs do not produce commercially sufficient quantities of AAV titers (e.g. resulting in fewer and poor-quality virions). Conversely, overexpressing VA RNA also results in a low titer of AAV virion production that would not be commercially feasible for scale-up. Thus, developing conditional VA RNA constructs, and combining any of those optimized constructs with the conditional helper constructs described herein, will provide commercially relevant, high-quality virions from the AAV production systems as described herein. FIG. 2C and FIGS. 4-15 illustrate various modified and inducible mutant VA RNA constructs and their effects on virion production. These various approaches provide numerous benefits over current systems for AAV production.

The constructs of this system can also be used in a vector system, wherein the constructs do not integrate into the genome of the cell.

### Conditional Expression

In a first aspect, the stable cell lines are provided. In some embodiments, the stable cell lines are mammalian stable cell lines. The cells are capable of conditionally producing recombinant AAV (rAAV) virions. In some embodiments, the cells are capable of conditionally producing rAAV virions. In some embodiments, said rAAV virions package an expressible payload. In some embodiments, said rAAV virions package a sequence encoding a payload. In preferred embodiments, production of virions is not conditioned on the presence of an episome or independent plasmid within the cell.

In another aspect, the plasmids or episomes are provided comprising the constructs as disclosed herein. In some embodiments, the plasmids or episomes are transfected mammalian stable cell lines. In some embodiments, the plasmids or episomes further comprise Epstein-Barr virus (EBV) sequences to stably maintain the constructs extrachromosomally. The cells are capable of conditionally producing recombinant AAV (rAAV) virions. In some embodiments, the cells are capable of conditionally producing rAAV virions. In some embodiments, said rAAV virions package an expressible payload. In some embodiments, said rAAV virions package a sequence encoding a payload.

In some embodiments, expression of AAV Rep is conditional. In some embodiments, expression of AAV Rep and Cap proteins is conditional. In certain embodiments, expression of AAV Rep and Cap proteins is conditioned on addition of at least a first expression triggering agent to the cell culture medium. In certain embodiments, expression of AAV Rep and Cap proteins is conditioned on addition of a first expression triggering agent and a second expression triggering agent to the cell culture medium.

In a system with a triggering agent, doxycycline is a suitable agent. In certain embodiments, doxycycline is used to the control a Tet inducible promoter. Alternatively, other inducible promoters can be utilized instead of a Tet inducible promoter, such as, but not limited to, a cumate inducible promoter system, which is under the control of cumate as the triggering agent or an ecdysone-inducible promoter, which is under the control of ecdysone or ponasterone as the triggering agent.

Any suitable inducible excising agent (e.g., recombinase) can be utilized. An excising agent can be a recombinase. An excising agent can be a site-specific recombinase. Exemplary site-specific recombinase systems include, without limitation, Cre-loxP, Flp-FRT, PhiC31-att, Dre-rox, and Tre-loxLTR site-specific recombinase systems. The Cre-loxP system uses a Cre recombinase to catalyze site-specific recombination between two loxP sites. The Flp-FRT system uses a flippase (FLP) recombinase to catalyze site-specific recombination between two flippase recognition target (FRT) sites. The PhiC31-att system uses a phiC31 recombinase to catalyze site-specific recombination between two attachment (att) sites referred to as attB and attP. The Dre-rox system uses a DreO recombinase to catalyze site-specific recombination between two rox sites. The Tre-loxLTR system uses a Tre recombinase to catalyze site-specific recombination between two loxP sites that are modified with HIV long terminal repeats (loxLTR). For a description of various site-specific recombinase systems, see, e.g., Stark et al. (2011) Biochem. Soc. Trans. 39 (2): 617-22; Olorunniji et al. (2016) Biochem. J. 473 (6): 673-684; Birling et al. (2009) Methods Mol. Biol. 561:245-63; García-Otin et al. (2006) Front. Biosci. 11:1108-1136; Weasner et al. (2017) Methods Mol. Biol. 1642:195-209; herein incorporated by reference in their entireties.

An excising agent can target a recombination site. Examples of suitable inducible excising agents include Cre and a flippase. The Cre element can be hormone activated Cre, or light inducible Cre. A recombination site can be a lox site. A lox site can be a loxP site. A recombination site can be an FRT site.

The Flippase recombinase system is based on Flp-FRT recombination, a site-directed recombination technology used to manipulate DNA under controlled conditions in vivo. It is analogous to Cre-lox recombination but involves the recombination of sequences between short flippase recognition target (FRT) sites by the recombinase flippase (Flp) derived from the 2μ plasmid of baker's yeast Saccharomyces cerevisiae. The Flp protein, much like Cre, is a tyrosine family site-specific recombinase.

In typical embodiments, the cells do not express cytotoxic levels of Rep protein prior to addition of both the first expression and second triggering agents to the cell culture medium. In certain embodiments, the cells do not express cytostatic levels of Rep protein prior to addition of both the first and second expression triggering agents to the cell culture medium. In certain embodiments, the average concentration of Rep protein within the cells is less than the amount prior to addition of both of the first and second expression triggering agents to the cell culture medium. In some embodiments, expression of Rep and Cap proteins becomes constitutive after addition of all of the at least first expression triggering agents to the cell culture medium.

In some embodiments, expression of at least one adenoviral helper protein is conditional.

In certain embodiments, expression of the at least one adenoviral helper protein is conditioned on addition of at least a third expression triggering agent to the cell culture medium. In particular embodiments, the third expression triggering agent is the same as the first expression triggering agent. In certain embodiments, expression of adenoviral helper proteins is conditioned on addition of a third expression triggering agent and a fourth expression triggering agent to the cell culture medium. In particular embodiments, the fourth expression triggering agent is the same as the second expression triggering agent. In particular embodiments, the third expression triggering agent is the same as the first expression triggering agent and the fourth expression triggering agent is the same as the second expression triggering agent.

In some embodiments, continued expression of adenoviral helper proteins following triggering of expression by contact of the cell with the at least third expression triggering agent requires the presence of only the third expression triggering agent in the cell culture medium. In certain embodiments, the third triggering agent is the same as the first triggering agent.

In some embodiments, expression of at least one adenoviral helper RNA is conditional. In certain embodiments, the adenoviral helper proteins comprise Ad E2A. In certain embodiments, the adenoviral helper proteins comprise Ad E4. In some embodiments, the adenoviral helper protein is tagged. A tag can be a protein tag. A protein tag can be a FLAG tag. In some embodiments, E2A is FLAG-tagged. In some embodiments, E4 is FLAG-tagged.

In particular embodiments, the adenoviral helper RNA is a VA RNA. In particular embodiments, the adenoviral helper RNA is expressed from an inducible VA RNA construct. In some embodiments, the VA RNA is a mutant VA RNA. In some embodiments, the VA RNA is a transcriptionally dead VA RNA. In some embodiments, the VA RNA is under the control of an RNA polymerase III promoter. In some embodiments, the VA RNA is under the control of an interrupted RNA polymerase III promoter. In some embodiments, the VA RNA is under the control of a U6 or U7 promoter. In some embodiments, the VA RNA is under the control of an interrupted U6 or U7 promoter.

In some embodiments, the third expression triggering agent is a tetracycline. In certain embodiments, the tetracycline is doxycycline (“Dox”). In some embodiments, the fourth expression triggering agent is an estrogen receptor ligand. In certain embodiments, the estrogen receptor ligand is a selective estrogen receptor modulator (SERM). In particular embodiments, the estrogen receptor ligand is tamoxifen.

In some embodiments of the stable cell line, expression of the payload is conditioned on addition of at least a fifth expression triggering agent to the cell culture medium. In some embodiments, expression of the payload is not conditioned on addition of an expression triggering agent to the cell culture medium.

In some embodiments, expression of Rep and Cap proteins, adenoviral helper proteins, and the payload becomes constitutive after addition of only one expression triggering agent to the cell culture medium. In certain embodiments, expression of Rep and Cap proteins and the adenoviral helper proteins becomes constitutive after addition of only one expression triggering agent to the cell culture medium.

In certain embodiments, the one expression triggering agent is the first expression triggering agent. In certain embodiments, the first expression triggering agent is a tetracycline. In particular embodiments, the first expression triggering agent is doxycycline.

### Synthetic Nucleic Acid Constructs

In typical embodiments, the nuclear genome of the cell of the stable cell line comprises a plurality of integrated synthetic nucleic acid constructs. Typically, each of the plurality of synthetic nucleic acid constructs is separately integrated into the nuclear genome of the cell. In some embodiments, only a single non-auxotrophic selection is required to maintain all of the plurality of synthetic nucleic acid constructs stably within the nuclear genome of the cells. In some embodiments, antibiotic resistance is required to maintain the plurality of synthetic constructs stably within the nuclear genomes of the cells. In some embodiments, both a non-auxotrophic selection and antibiotic resistance is required to maintain the plurality of synthetic constructs stably within the nuclear genomes of the cells. In some embodiments, auxotrophic selection and antibiotic resistance is required to maintain the plurality of synthetic constructs stably within the nuclear genomes of the cells. In some embodiments, auxotrophic selection is required to maintain the plurality of synthetic constructs stably within the nuclear genomes of the cells.

In some embodiments, the nuclear genome of the cell comprises two integrated synthetic constructs.

In some embodiments, the nuclear genome of the cell comprises three integrated synthetic constructs. In particular embodiments, the first integrated synthetic construct comprises conditionally expressible AAV Rep and Cap coding sequences; the second integrated synthetic construct comprises a conditionally expressible Cre coding sequence and conditionally expressible adenoviral helper protein coding sequences; and the third integrated synthetic construct comprises expressible coding sequences for the payload.

In some embodiments, the nuclear genome of the cell comprises four or more integrated synthetic constructs. In particular embodiments, the four or more integrated synthetic construct comprises, in various combinations, conditionally expressible AAV Rep coding sequences; conditionally expressible Cap coding sequences; a recombinase coding sequence (e.g., can be under a constitutive or inducible promoter; may or may not be self-excising); conditionally expressible adenoviral helper protein coding sequences; the expressible coding sequences for the payload; and expressible VA RNA coding sequence (e.g., can be under a constitutive or inducible promoter; can be wild-type VA RNA or a mutant thereof).

### AAV Rep/Cap Construct(s)

Disclosed herein are polynucleotide constructs encoding for a Rep and Cap polypeptide. Provided herein is a first polynucleotide construct, which encodes for Rep and Cap and comprises spacer or excisable elements. This first polynucleotide construct (Construct 1) is also referred to as a Rep/Cap construct, and/or “AAV Rep/Cap Construct.” In some embodiments, the elements of this first polynucleotide construct are in one or more separate constructs.

These polynucleotide constructs can be stably integrated into a cell line and are triggered to produce AAV Rep and Cap polypeptides in the presence of an excising element (e.g., a recombinase). In some embodiments, the first integrated synthetic construct comprises conditionally expressible AAV Rep and Cap coding sequences. In some embodiments, the polynucleotides do not integrate into the cell line and are triggered to produce AAV Rep and Cap polypeptides in the presence of an excising element (e.g., a recombinase. In some embodiments, the transfected plasmids or episomes comprise conditionally expressible AAV Rep and Cap coding sequences.

The Rep sequence can encode Rep from any desired AAV serotype. In some embodiments, the encoded Rep protein is drawn from the same serotype as the Cap protein. In some embodiments, the encoded Rep protein is drawn from a different serotype from the Cap protein. In particular embodiments, the encoded Rep protein includes, but is not limited to, a Rep protein from AAV serotypes AAV-1, AAV-2, AAV-3, AAV-4, AAV-5, AAV-6, AAV-7, AAV-8, AAV-9, AAV-10 and AAV-11, or chimeric combinations thereof.

The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol, 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al. Virol, 78:6381-6388 (2004); the AAV-10 genome is provided in Mol Ther, 13 (1): 67-76 (2006); and the AAV-11 genome is provided in Virology, 330 (2): 375-383 (2004).

In the exemplary embodiments illustrated in FIG. 3A, prior to the cell being contacted with the first expression triggering agent, the Rep coding sequence is interrupted by an intervening spacer. In some embodiments, the intervening spacer is inserted at CAG-G, CAG-A, AAG-G, AAG-A, wherein the dash (−) indicates the point of insertion of the intervening spacer, in the Rep coding sequence, and the intervening spacer is inserted downstream of the p19 promoter and upstream of the p40 promoter. In some embodiments, the intervening spacer is an excisable spacer.

In certain embodiments, the intervening spacer segment comprises, from 5′ to 3′, a first spacer segment, a second spacer segment, and a third spacer segment. In some embodiments, the intervening spacer is inserted at CAG-G, CAG-A, AAG-G, AAG-A, wherein the dash (−) indicates the point of insertion of the intervening spacer, in the Rep coding sequence, and the intervening spacer is inserted downstream of the p19 promoter and upstream of the p40 promoter. In some embodiments, the intervening spacer is an excisable spacer.

In particular embodiments, the first spacer segment comprises a 5′ splice site (5′SS) 5′ to the first spacer element. In some embodiments, the first spacer segment comprises a nucleic acid sequence having at least 80% identity to SEQ ID NO: 1.

In some embodiments, the second spacer segment comprises a polynucleotide encoding a detectable protein marker flanked by lox sites. In certain embodiments, the detectable protein marker is a fluorescent protein. In particular embodiments, the fluorescent protein is a green fluorescent protein (GFP). In specific embodiments, the GFP is EGFP. In particular embodiments, the fluorescent protein is a blue fluorescent protein (BFP). Screening for the fluorescent marker can be used to confirm integration of the construct into the cell genome, and can subsequently be used to confirm excision of the intervening spacer segment. In some embodiments, the second spacer segment further comprises a polyA sequence. In certain embodiments, the poly A sequence comprises a rabbit beta globin (RBG) polyA. In some embodiments, the second spacer segment further comprises a first 3′ splice site (3′SS) between the first lox site and the polynucleotide encoding the protein marker.

In some embodiments, the second spacer segment comprises a nucleic acid sequence having at least 80% identity to SEQ ID NO: 2.

In some embodiments, the third spacer segment further comprises a second 3′ splice site (3′SS). In particular embodiments, the second 3′ splice site is positioned 3′ to the second lox site.

In some embodiments, the third spacer segment comprises a nucleic acid sequence having at least 80% identity to SEQ ID NO: 3.

In various embodiments, the Rep coding sequences are operatively linked to an endogenous P5 promoter. In various embodiments, the Rep coding sequences are operatively linked to an endogenous P19 promoter. In some embodiments, the intervening spacer is inserted into the Rep coding sequence at a position downstream of the P19 promoter and upstream of the endogenous P40 promoter. In some embodiments, the intervening spacer is inserted at CAG-G, CAG-A, AAG-G, AAG-A downstream of the P19 promoter and upstream of the P40 promoter, wherein the dash (−) indicates the point of insertion of the intervening spacer, in the Rep coding sequence.

In some embodiments, the Rep coding sequences are operably linked to an inducible promoter. In some embodiments, the inducible promoter comprises a tetracycline-inducible promoter, a cumate-inducible promoter, or a cumate-inducible promoter. In some embodiments, the Rep coding sequences are operably linked to a constitutive promoter. In some embodiments, the constitutive promoter is EF1alpha promoter or human cytomegalovirus promoter. In some embodiments, the intervening spacer is inserted into the Rep coding sequence at a position downstream of the P19 promoter and upstream of the endogenous P40 promoter. In some embodiments, the intervening spacer is inserted at CAG-G, CAG-A, AAG-G, AAG-A downstream of the P19 promoter and upstream of the P40 promoter, wherein the dash (−) indicates the point of insertion of the intervening spacer, in the Rep coding sequence.

In some embodiments, the Rep coding sequence is 5′ to the Cap coding sequence. In certain embodiments, the Cap coding sequence is operatively linked to an endogenous P40 promoter.

In some embodiments, the Cap coding sequence is operably linked to a promoter. In some embodiments, the sequence coding for VP1, the sequence coding for VP2, and the sequence coding for VP3 are operably linked to a promoter. In some embodiments, a single construct or separate constructs comprise these sequences, in any combination. In some embodiments, the promoter is an inducible promoter. In some embodiments, the inducible promoter comprises a tetracycline-inducible promoter, a cumate-inducible promoter, or a cumate-inducible promoter. In some embodiments, the promoter is a constitutive promoter, wherein the sequences coding for the one or more cap proteins are downstream of an excisable element (e.g., a sequence flanked by recombination sites and comprising a stop signal) and constitutive promoter, wherein upon excision of the excisable element (e.g., by a recombinase), the sequences coding for the one or more cap proteins are operably linked to the constitutive promoter. In some embodiments, the constitutive promoter is EF1alpha promoter or human cytomegalovirus promoter.

In various embodiments, the Cap protein is selected from the capsid of an avian AAV, bovine AAV, canine AAV, equine AAV, primate AAV, non-primate AAV, and ovine AAV, and modifications, derivatives, or pseudotypes thereof.

In some embodiments, the capsid is a capsid selected from AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 or AAVhu68 (described in WO2020/033842, incorporated herein by reference in its entirety). The hu68 capsid is described in WO 2018/160582, incorporated herein by reference in its entirety.

In some embodiments, the capsid is a derivative, modification, or pseudotype of AAV1, AAV2, AAV3, AAV4, AAV5, AAV6, AAV7, AAV8, AAV9, AAV 10, AAV11, AAV 12, AAV 13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16 or AAVhu68.

In some embodiments, capsid protein is a chimera of capsid proteins from two or more serotype selected from AAV1, AAV2, rAAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16 (described in WO2020/033842, incorporated herein by reference in its entirety). In certain embodiments, the capsid is an rh32.33 capsid, described in U.S. Pat. No. 8,999,678, incorporated herein by reference in its entirety.

In particular embodiments, the capsid is an AAV1 capsid. In particular embodiments, the capsid is an AAV5 capsid. In particular embodiments, the capsid is an AAV9 capsid.

In various embodiments, the first integrated construct further comprises a first mammalian cell selection element.

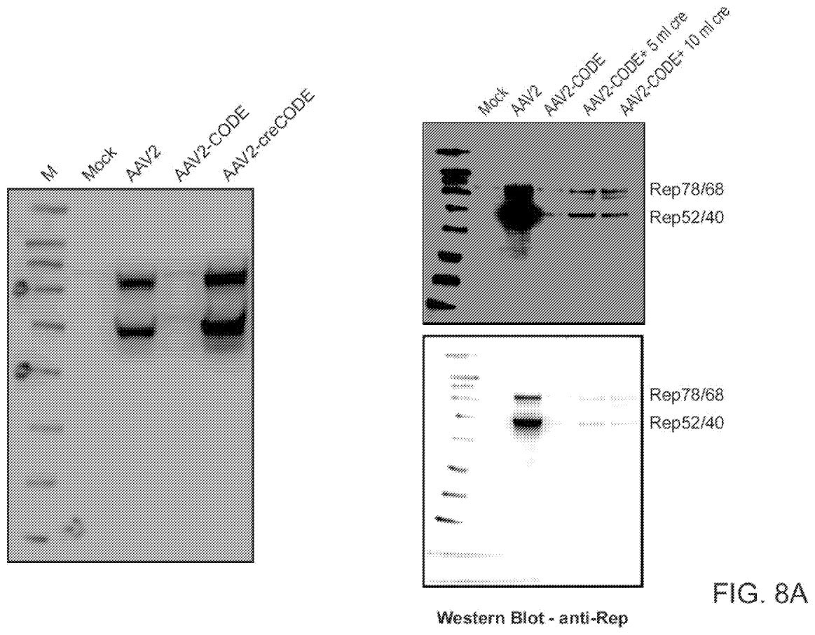

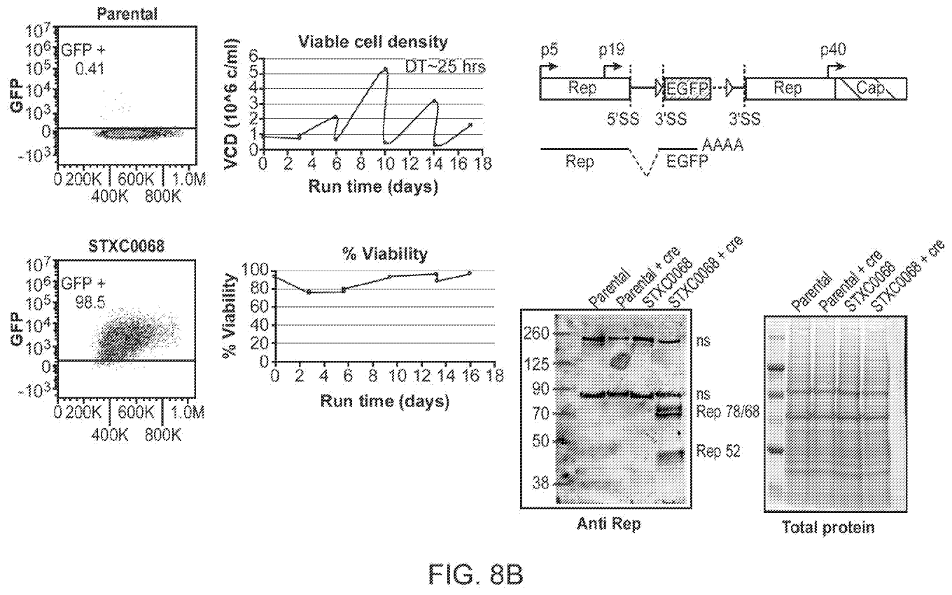

In some embodiments, the inducible Rep and Cap construct is as shown in FIG. 8B. In some embodiments, the inducible polynucleotide construct encoding for Rep and Cap encodes for a first part of a Rep polypeptide, a second part of a Rep polypeptide, a Cap polypeptide, and an excisable element. The excisable element may be positioned between the first part of the Rep polypeptide and the second part of the Rep polypeptide. The excisable element may, thus, interrupt the sequence encoding for the Rep polypeptide at any point along the sequence encoding for Rep. Without excision of the excisable element, Rep is minimally expressed or not expressed at all. In some embodiments, the Rep polypeptide is a wildtype Rep polypeptide. In other embodiments, the Rep polypeptide is a mutant Rep polypeptide. In some embodiments, the Cap polypeptide is a wildtype Cap polypeptide. In other embodiments, the Cap polypeptide is a mutant Cap polypeptide. In some embodiments, the excisable element comprises an intron, an exon, or an intron and an exon. In particular embodiments, the excisable element from 5′ to 3′ comprises a 5′ splice site; a first spacer segment comprising a first intron; a second spacer segment comprising: a first lox sequence, a 3′ splice site, an exon, a stop signaling sequence, a second lox sequence; and a third spacer segment comprising a second intron. The first spacer segment and the third spacer segment may be excised by endogenous cellular machinery.

In some embodiments, the second spacer segment in the excisable element is excised by a recombinase. A recombinase can be Cre. Cre may be provided as any form of exogenous Cre, such as Cre gesicles. Cre may also be encoded for by a second polynucleotide construct or by any separate polynucleotide construct. In some embodiments, a construct encoding for adenoviral helper proteins also encodes for Cre. In some embodiments, the second polynucleotide construct is also inducible, for example, as described below. In some embodiments, a construct encoding for Rep/Cap proteins also encodes for Cre.

In some embodiments, expression of the Rep and Cap are driven by native promoters, including P5, P19, P40, or any combination thereof. In some embodiments, expression of the Rep and Cap are driven by inducible promoters. In some embodiments, expression of the Rep and Cap are driven by constitutive promoters. In some embodiments the exon of the excisable element may be any detectable marker. For example, detectable markers contemplated herein include luminescent markers, fluorescent markers, or radiolabels. Fluorescent markers include, but are not limited to, EGFP, GFP, BFP, RFP, or any combination thereof.

In some embodiments, the Rep/Cap construct is a polynucleotide construct comprising: a) a sequence of a first part of a Rep gene; b) sequence of a second part of the Rep gene; c) a sequence of a Cap gene; and d) an excisable element positioned between the first part of the sequence of Rep gene and the second part of the sequence of the Rep gene. In some embodiments, the excisable element comprises a stop signaling sequence. In some embodiments, the excisable element comprises a rabbit beta globin intron. In some embodiments, the excisable element comprises an exon. In some embodiments, the excisable element comprises an intron and an exon. In some embodiments, the excisable element comprises an intron. In some embodiments, two splice sites are positioned between the sequence of the first part of the Rep gene and the sequence of the second part of the Rep gene. In some embodiments, the two splice sites are a 5′ splice site and a 3′ splice site. In some embodiments, the 5′ splice site is a rabbit beta globin 5′ splice site. In some embodiments, the 3′ splice site is a rabbit beta globin 3′ splice site. In some embodiments, three splice sites are positioned between the sequence of the first part of the Rep gene and the sequence of the second part of the Rep gene. In some embodiments, the three splice sites are a 5′ splice site, a first 3′ splice site, and a second 3′ splice site. In some embodiments, a first 3′ splice site is a duplicate of the second 3′ splice site. In some embodiments, the first 3′ splice site is a rabbit beta globin 3′ splice site. In some embodiments, the second 3′ splice site is a rabbit beta globin 3′ splice site. In some embodiments, the excisable element comprises a recombination site. In some embodiments, the recombination site is a lox site or FRT site. In some embodiments, the lox site is a loxP site. In some embodiments, the excisable element comprises from 5′ to 3′: a) the 5′ splice site; b) a first recombination site; c) the first 3′ splice site; d) a stop signaling sequence; e) a second recombination site; and f) the second 3′ splice site. In some embodiments, the excisable element comprises from 5′ to 3′: a) the 5′ splice site; b) a first spacer segment; c) a second spacer segment comprising: i) a first recombination site; ii) the first 3′ splice site; iv) a stop signaling sequence; and v) a second recombination site; and d) a third spacer segment comprising the second 3′ splice site. In some embodiments, the first spacer sequence comprises an intron. In some embodiments, the first spacer segment comprises a sequence having at least 70%, 80%, 90%, 95%, 99%, or 100% sequence identity to SEQ ID NO: 1. In some embodiments, the second spacer segment comprises a sequence having at least 70%, 80%, 90%, 95%, 99%, or 100% sequence identity to SEQ ID NO: 2. In some embodiments, the third spacer segment comprises a sequence having at least 70%, 80%, 90%, 95%, 99%, or 100% sequence identity to SEQ ID NO: 3. In some embodiments, the third spacer segment comprises an intron. In some embodiments, the first spacer segment and the third spacer segment are capable of being excised by endogenous cellular machinery. In some embodiments, the second spacer segment comprises an exon. In some embodiments, the second spacer segment further comprises a poly A sequence. In some embodiments, the polyA sequence is 3′ of the exon. In some embodiments, the polyA sequence comprises a rabbit beta globin (RBG) polyA sequence. The polynucleotide construct of any one of claims, wherein the second spacer segment comprises from 5′ to 3′: a) a first recombination site; b) the first 3′ splice site; c) an exon; d) a stop signaling sequence; and e) a second recombination site. In some embodiments, the first recombination site is a first lox sequence and the second recombination site is a second lox sequence. In some embodiments, the first lox sequence is a first loxP sequence and a second lox sequence is a second loxP sequence. In some embodiments, the first recombination site is a first FRT site and the second recombination site is a second FRT site. In some embodiments, the stop signaling sequence is a termination codon of the exon or a polyA sequence. In some embodiments, the poly A sequence comprises a rabbit beta globin (RBG) polyA sequence. In some embodiments, the exon encodes a detectable marker or a selectable marker. In some embodiments, the detectable marker comprises a luminescent marker or a fluorescent marker. In some embodiments, the fluorescent marker is GFP, EGFP, RFP, CFP, BFP, YFP, or mCherry. In some embodiments, the second spacer segment is excisable by a recombinase. In some embodiments, the recombinase is a site-specific recombinase. In some embodiments, the recombinase is a Cre polypeptide or a Flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the recombinase is encoded by a second polynucleotide construct or exogenously provided. In some embodiments, the Rep gene codes for Rep polypeptides. In some embodiments, the Cap gene codes for Cap polypeptides. In some embodiments, transcription of the Rep gene and the Cap gene are driven by native promoters. In some embodiments, the native promoters comprise P5, P19, and P40. In some embodiments, transcription of the Rep gene and the Cap gene are driven by inducible promoters. In some embodiments, the Rep polypeptides are wildtype Rep polypeptides. In some embodiments, the Rep polypeptides comprise Rep78, Rep68, Rep52, and Rep40. In some embodiments, a truncated replication associated protein comprising a polypeptide expressed from the sequence of first part of a Rep gene and the exon is capable of being expressed in the absence of the recombinase. In some embodiments, the Cap polypeptides are wildtype Cap polypeptides. In some embodiments, the Cap polypeptides are AAV capsid proteins. In some embodiments, the AAV capsid proteins comprise VP1, VP2, and VP3. In some embodiments, a serotype of the AAV capsid proteins is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68.

In some embodiments, the Rep/Cap construct further comprises a sequence coding for a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the Rep/Cap construct further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the Rep/Cap construct can increase helper enzyme levels thereby increasing production of the molecule required for cell growth, by, e.g., increasing levels of a co-factor required for enzyme activity. In some embodiments, the Rep/Cap construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, a selectable marker and helper enzyme comprises at least 80%, 85%, 90%, 95%, 98%, 99%, or 100% sequence identity to SEQ ID NO: 101-SEQ ID NO: 109.

In some embodiments, the Rep/Cap construct is in a vector. In some embodiments, the Rep/Cap construct is in a plasmid. In some embodiments, the Rep/Cap construct is in a bacterial artificial chromosome or yeast artificial chromosome. In some embodiments, the Rep/Cap construct is a synthetic nucleic acid construct. In some embodiments, the Rep/Cap construct comprises a sequence having at least 70%, 80%, 90%, 95%, 99%, or 100% sequence identity to any one of SEQ ID NO: 1-SEQ ID NO: 3, SEQ ID 6-SEQ ID NO: 8, SEQ ID NO: 32, SEQ ID NO: 90-SEQ ID NO: 99, SEQ ID NO: 101-SEQ ID NO: 109, SEQ ID NO: 112-SEQ ID NO: 131, or SEQ ID NO: 136-SEQ ID NO: 138, or any combination thereof. In some embodiments, the Rep/Cap construct has at least 70%, 80%, 90%, 95%, 99%, or 100% sequence identity to any one of SEQ ID NO: 1-SEQ ID NO: 3, SEQ ID 6-SEQ ID NO: 8, SEQ ID NO: 32, SEQ ID NO: 90-SEQ ID NO: 99, SEQ ID NO: 101-SEQ ID NO: 109, SEQ ID NO: 112-SEQ ID NO: 131, or SEQ ID NO: 136-SEQ ID NO: 138, or any combination thereof. In some embodiments, the Rep/Cap construct comprises SEQ ID NO: 145 downstream of the sequence encoding the AAV Cap proteins. In some embodiments, the Rep/Cap construct lacks SEQ ID NO: 145 downstream of the sequence encoding the AAV Cap proteins. In some embodiments, the Rep/Cap construct comprises a sequence having at least 70%, 80%, 90%, 95%, 99%, or 100% sequence identity to any one of SEQ ID NO: 1-SEQ ID NO: 3, SEQ ID 6-SEQ ID NO: 8, SEQ ID NO: 32, SEQ ID NO: 90-SEQ ID NO: 99, SEQ ID NO: 101-SEQ ID NO: 109, SEQ ID NO: 112-SEQ ID NO: 131, or SEQ ID NO: 136-SEQ ID NO: 138, but wherein Rep/Cap construct lacks SEQ ID NO: 145 downstream of the sequence encoding the AAV Cap proteins.

In some embodiments, the Rep/Cap construct further comprises a sequence coding for VA RNA. In some embodiments, a sequence coding for VA RNA is in separate construct or in any separate construct coding for an element of the Rep/Cap construct. In some embodiments, a payload construct comprises a polynucleotide construct coding for a VA RNA. In some embodiments, the VA RNA is operably linked to a constitutive promoter or an inducible promoter. In some embodiments, the constitutive promoter is EF1alpha promoter or human cytomegalovirus promoter. In some embodiments, the inducible promoter is a tetracycline-inducible promoter, an ecdysone-inducible promoter, or a cumate-inducible promoter. In some embodiments, the sequence coding for VA RNA is a transcriptionally dead sequence. In some embodiments, the sequence coding for VA RNA comprises at least two mutations in the internal promoter. In some embodiments, the expression of the VA RNA is under the control of an RNA polymerase III promoter. In some embodiments, the expression of the VA RNA is under the control of an interrupted RNA polymerase III promoter. In some embodiments, the expression of the VA RNA is under the control of a U6 or U7 promoter. In some embodiments, the expression of the VA RNA is under the control of an interrupted U6 or U7 promoter. In some embodiments, the polynucleotide construct comprises upstream of the sequence coding for VA RNA gene sequence, from 5′ to 3′: a) a first part of a U6 or U7 promoter sequence; b) a first recombination site; c) a stuffer sequence; d) a second recombination site; e) a second part of a U6 or U7 promoter sequence. In some embodiments, the stuffer sequence is excisable by the recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter.

In some embodiments the gene codes for a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the stuffer sequence further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the stuffer sequence further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

A major advantage of the inducible polynucleotide constructs disclosed herein encoding for Rep and Cap include that upon stable integration into a mammalian cell line, expression of Rep and Cap is inducible even in the absence of a transfection agent or a plasmid. In some embodiments, the stable cell line populations disclosed herein are homogeneous. For example, at least 70%, at least 80%, at least 90%, at least 95%, or 100% of the stable cell population comprises the stably integrated polynucleotide construct encoding for Rep and Cap proteins.

### Adenoviral Helper Construct(s)

Provided herein is a second polynucleotide construct (also referred to as Construct 2), which encodes for one or more adenoviral helper proteins. This second polynucleotide construct is also referred to as an inducible helper construct (e.g., Adenoviral Helper Construct provides one or more helper proteins selected from E1A, E1B, E2A and E4 absent from a host cell) to be used in production of rAAV virions. In some embodiments, the sequence encoding E4 is a sequence encoding E4orf6. In some embodiments, the elements of an inducible helper construct (e.g., one or more helper proteins selected from E1A, E1B, E2A and E4 absent from a host cell) are in one or more separate constructs to be used in production of rAAV virions. In some embodiments, the host cell provides, one, two, or three of the four helper proteins. For example, for a host cell expressing E1A and E1B, the adenoviral helper construct provides E2A and E4. For a host cell expressing E2A and E4, the adenoviral helper construct provides E1A and E1B. For a host cell expressing E1B, the adenoviral helper construct provides E1A, E2A and E4. For a host cell expressing E2A, the adenoviral helper construct provides E1B, E1A and E4. For a host cell expressing E4, the adenoviral helper construct provides E1B, E2A and E1A. For a host cell expressing E1A, E2A and E4, the adenoviral helper construct provides E1B. For a host cell expressing E1B, E1A and E4, the adenoviral helper construct provides E2A. For a host cell expressing E1B, E2A and E1A, the adenoviral helper construct provides E4. In some embodiments, E4 is E4orf6.

In some embodiments, the sequences coding for E1A, E1B, E2A, and E4 are operably linked to separate promoters. In some embodiments, the sequences coding for E1A, E1B, E2A, and E4 are operably linked to one promoter. In some embodiments, the sequences coding for E1A, E1B, E2A, and E4 are operably linked, in any combination, to one promoter or separate promoters. The separate promoters can be the same promoters or different promoters. A combination of the separate promoters and the one promoter can be the same promoters or different promoters. The one promoter can be a native promoter, a constitutive promoter, or an inducible promoter. The separate promoters can be native promoters, constitutive promoters, inducible promoters, or any combination thereof. In some embodiments, the constitutive promoter is EF1alpha promoter or human cytomegalovirus promoter. In some embodiments, the inducible promoter is a tetracycline-inducible promoter, a cumate-inducible promoter, or an ecdysone-inducible promoter. For example, some the sequence coding for E1A and E1B are separated by an IRES sequence of P2A sequence and are operably linked to one promoter. In some embodiments, the sequence coding for E1A and E2A are separated by an IRES sequence of P2A sequence and are operably linked to one promoter. In some embodiments, the sequence coding for E1A and E4 are separated by an IRES sequence of P2A sequence and are operably linked to one promoter. In some embodiments, the sequence coding for E1B and E2A are separated by an IRES sequence of P2A sequence and are operably linked to one promoter. In some embodiments, the sequence coding for E1B and E4 are separated by an IRES sequence of P2A sequence and are operably linked to one promoter. In some embodiments, the sequence coding for E2A and E4 are separated by an IRES sequence of P2A sequence and are operably linked to one promoter. In some embodiments, the sequences coding for the helper proteins are in different orientations. In some embodiments, the sequences coding for the helper proteins are bidirectional. In some embodiments, the E1A is operably linked to a natural or constitutive promoter, E1B is operably linked to a natural or constitutive promoter, and E2A and E4 are downstream of an excisable element (e.g., a sequence flanked by recombination sites and comprising a stop signal) and constitutive promoter, wherein upon excision of the excisable element (e.g., by a recombinase), E2A and E4 are operably linked to the constitutive promoter. In some embodiments, the E1A is operably linked to a natural or constitutive promoter, E1B is operably linked to a natural or constitutive promoter, and E2A and E4 are downstream of an excisable element (e.g., a sequence flanked by recombination sites and comprising a stop signal) and inducible promoter, wherein upon excision of the excisable element (e.g., by a recombinase), E2A and E4 are operably linked to the inducible promoter. In some embodiments, the E2A is operably linked to a natural or constitutive promoter, E4 is operably linked to a natural or constitutive promoter, and E1A and E1B are downstream of an excisable element (e.g., a sequence flanked by recombination sites and comprising a stop signal) and constitutive promoter, wherein upon excision of the excisable element (e.g., by a recombinase), E1A and E1B are operably linked to the constitutive promoter. In some embodiments, the E2A is operably linked to a natural or constitutive promoter, E4 is operably linked to a natural or constitutive promoter, and E1A and E1B are downstream of an excisable element (e.g., a sequence flanked by recombination sites and comprising a stop signal) and inducible promoter, wherein upon excision of the excisable element (e.g., by a recombinase), E1A and E1B are operably linked to the inducible promoter.

In certain embodiments, the adenoviral helper construct provides inducible production of one or more of the helper proteins. In some embodiments, an adenoviral helper protein further comprises a protein tag. A protein tag can be a FLAG tag. In some embodiments, E2A is a FLAG tagged E2A. In some embodiments, E4 is a FLAG tagged E4. A protein tag, such as a FLAG tag, can be used to screen for or to confirm integration of the second polynucleotide construct and expression of the adenoviral helper protein from the second polynucleotide construct in a cell after induction.

In some embodiments, the second integrated synthetic construct comprises conditionally expressible recombinase and conditionally expressible adenovirus helper proteins. In some embodiments, the second synthetic construct comprises conditionally expressible recombinase and conditionally expressible adenovirus helper proteins. In some embodiments, the one or more separate integrated constructs comprises conditionally expressible recombinase and conditionally expressible adenovirus helper proteins. In some embodiments, the one or more separate constructs comprises conditionally expressible recombinase and conditionally expressible adenovirus helper proteins. In some embodiments, the second integrated synthetic construct comprises conditionally expressible Cre recombinase and conditionally expressible adenovirus helper proteins. In the exemplary embodiments illustrated in FIG. 2A, prior to the cell being contacted with at least a third expression triggering agent, the second integrated construct comprises, from 5′ to 3′: an inducible promoter, a Cre coding sequence, a first polyA sequence, adenoviral helper protein coding sequences, a second polyA sequence, a constitutive promoter, a coding sequence for a protein that is responsive to the first expression triggering agent, and a second mammalian cell selection element.

In typical embodiments, the Cre coding sequence is operatively linked to the inducible promoter. In various embodiments, the inducible promoter comprises an element responsive to the third expression triggering agent. In some embodiments, the inducible promoter contains a regulatory sequence that allows for control of the promoter. The regulatory sequence can be operably linked to the promoter and positioned upstream of the promoter. Such regulatory sequences are known to those of skill in the art, and examples include those which cause the expression of a gene to be turned on or off in response to a chemical or physical stimulus, including the presence of a regulatory compound. The regulatory sequence used to control expression may be endogenous or exogenous to the host cell. In some embodiments, bacterial gene control elements in combination with viral transactivator proteins are used to provide mammalian inducible expression. Examples of mammalian-compatible regulatory sequences include those capable of controlling an engineered promoter to adjust transcription in response to antibiotics including, without limitation, tetracyclines, streptogramins, and macrolides. For a description of various inducible expression systems, see, e.g., Weber et al. (2004) Methods Mol. Biol. 267:451-66, Das et al. (2016) Curr. Gene Ther. 16 (3): 156-67, Chruscicka et al. (2015) J. Biomol. Screen. 20 (3): 350-8, Yarranton (1992) Curr. Opin. Biotechnol. 3 (5): 506-11, Gossen & Bujard (1992) Proc. Natl. Acad. Sci. U.S.A. 89 (12): 5547-51, Gossen et al. (1995) Science 268 (5218): 1766-9; herein incorporated by reference.

In some embodiments, a bacterial tetracycline response element (TRE) is included in a construct to allow mammalian expression to be induced by tetracycline or a derivative thereof (e.g., doxycycline). In certain embodiments, the inducible promoter comprises a plurality of tetracycline (Tet) operator elements capable of binding to a Tet responsive activator protein in the presence of a tetracycline. In some embodiments, the plurality of tetracycline (Tet) operator elements form a Tetracycline Responsive element (TRE). In some embodiments, the TRE comprises seven repeats of a 19 base pair operator sequence. In further embodiments, the TRE comprises seven repeats of a 19 base pair operator sequence upstream of a minimal CMV promoter sequence.

In some embodiments, a Tet responsive activator protein comprises an amino acid sequence having at least 90% identity (e.g., at least 95% or 100% identity) to the amino acid

(SEQ ID NO: 40)
MSRLDKSKVINSALELLNGVGIEGLTTRKLAQKLGVEQPTLYWHVKNKR
ALLDALPIEMLDRHHTHFCPLEGESWQDFLRNNAKSFRCALLSHRDGAK
VHLGTRPTEKQYETLENQLAFLCQQGFSLENALYALSAVGHFTLGCVLE
EQEHQVAKEERETPTTDSMPPLLRQAIELFDRQGAEPAFLFGLELIICG
LEKQLKCESGGPADALDDFDLDMLPADALDDFDLDMLPADALDDFDLDM
LPG.

In some embodiments, a Tet responsive activator protein comprises a nucleic acid sequence having at least 90% identity (e.g., at least 95% or 100% identity) to the amino acid sequence:

(SEQ ID NO: 69)
ATGTCTAGACTGGACAAGAGCAAAGTCATAAACTCTGCTCTGGAATTAC
TCAATGGAGTCGGTATCGAAGGCCTGACGACAAGGAAACTCGCTCAAAA
GCTGGGAGTTGAGCAGCCTACCCTGTACTGGCACGTGAAGAACAAGCGG
GCCCTGCTCGATGCCCTGCCAATCGAGATGCTGGACAGGCATCATACCC
ACTTCTGCCCCCTGGAAGGCGAGTCATGGCAAGACTTTCTGCGGAACAA
CGCCAAGTCATTCCGCTGTGCTCTCCTCTCACATCGCGACGGGGCTAAA
GTGCATCTCGGCACCCGCCCAACAGAGAAACAGTACGAAACCCTGGAAA
ATCAGCTCGCGTTCCTGTGTCAGCAAGGCTTCTCCCTGGAGAACGCACT
GTACGCTCTGTCCGCCGTGGGCCACTTTACACTGGGCTGCGTATTGGAG
GAACAGGAGCATCAAGTAGCAAAAGAGGAAAGAGAGACACCTACCACCG
ATTCTATGCCCCCACTTCTGAGACAAGCAATTGAGCTGTTCGACCGGCA
GGGAGCCGAACCTGCCTTCCTTTTCGGCCTGGAACTAATCATATGTGGC
CTGGAGAAACAGCTAAAGTGCGAAAGCGGCGGGCCGGCCGACGCCCTTG
ACGATTTTGACTTAGACATGCTCCCAGCCGATGCCCTTGACGACTTTGA
CCTTGATATGCTGCCTGCTGACGCTCTTGACGATTTTGACCTTGACATG
CTCCCCGGGTAA.

In some embodiments, a Tet responsive activator protein comprises a nucleic acid sequence having at least 90% identity (e.g., at least 95% or 100% identity) to the amino acid

(SEQ ID NO: 70)
ATGTCTAGATTAGATAAAAGTAAAGTGATTAACAGCGCATTAGAGCTGC
TTAATGGGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTACCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGATGGAGCAAAA
GTACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCACTTTACTTTAGGTTGCGTATTGGAA
GAACAAGAGCATCAAGTCGCTAAAGAAGAAAGGGAAACACCTACTACTG
ATAGTATGCCGCCATTATTACGACAAGCTATCGAATTATTTGATCGCCA
AGGTGCAGAGCCAGCCTTCTTATTCGGCCTTGAATTGATCATATGCGGA
TTAGAAAAACAACTTAAATGTGAAAGTGGGTCCGCGTACAGCCGCGCGC
GTACGAAAAACAATTACGGGTCTACCATCGAGGGCCTGCTCGATCTCCC
GGACGACGACGCCCCCGAAGAGGCGGGGCTGGCGGCTCCGCGCCTGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein comprises a nucleic acid sequence having at least 90% identity (e.g., at least 95% or 100% identity) to the amino acid sequence:

(SEQ ID NO: 71)
ATGTCTAGATTAGATAAAAGTAAAGTGATTAACAGCGCATTAGAGCTGC
TTAATGGGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTACCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGATGGAGCAAAA
GTACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCATTTTACTTTAGGTTGCGTATTGGAA
GATCAAGAGCATCAAGTCGCTAAAGAAGAAAGGGAAACACCTACTACTG
ATAGTATGCCGCCATTATTACGACAAGCTATCGAATTATTTGATCACCA
AGGTGCAGAGCCAGCCTTCTTATTCGGCCTTGAATTGATCATATGCGGA
TTAGAAAAACAACTTAAATGTGAAAGTGGGTCCGCGTACAGCCGCGCGC
GTACGAAAAACAATTACGGGTCTACCATCGAGGGCCTGCTCGATCTCCC
GGACGACGACGCCCCCGAAGAGGCGGGGCTGGCGGCTCCGCGCCTGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein comprises a nucleic acid sequence having at least 90% identity (e.g., at least 95% or 100% identity) to the amino acid

(SEQ ID NO: 72)
ATGTCTAGATTAGATAAAAGTAAAGTGATTAACGGCGCATTAGAGCTGC
TTAATGGGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTACCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGATGGAGCAAAA
GTACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCATTTTACTTTAGGTTGCGTATTGGAA
GATCAAGAGCATCAAGTCGCTAAAGAAGAAAGGGAAACACCTACTACTG
ATAGTATGCCGCCATTATTACGACAAGCTATCGAATTATTTGATCACCA
AGGTGCAGAGCCAGCCTTCTTATTCGGCCTTGAATTGATCATATGCGGA
TTAGAAAAACAACTTAAATGTGAAAGTGGGTCCGCGTACAGCCGCGCGC
GTACGAAAAACAATTACGGGTCTACCATCGAGGGCCTGCTCGATCTCCC
GGACGACGACGCCCCCGAAGAGGCGGGGCTGGCGGCTCCGCGCCTGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein comprises a nucleic acid sequence having at least 90% identity (e.g., at least 95% or 100% identity) to the amino acid

(SEQ ID NO: 73)
ATGTCTAGATTAGATAAAAGTAAAGTGATTAACGGCGCATTAGAGCTGC
TTAATGGGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTACCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGATGGAGCAAAA
GTACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCACTTTACTTTAGGTTGCGTATTGGAA
GAACAAGAGCATCAAGTCGCTAAAGAAGAAAGGGAAACACCTACTACTG
ATAGTATGCCGCCATTATTACGACAAGCTATCGAATTATTTGATCGCCA
AGGTGCAGAGCCAGCCTTCTTATTCGGCCTTGAATTGATCATATGCGGA
TCTCCCGGACGACGACGCCCCCGAAGAGGCGGGGCTGGCGGCTCCGCGC
CTTAGAAAAACAACTTAAATGTGAAAGTGGGTCCGCGTACAGCCGCGCG
CGTACGAAAAACAATTACGGGTCTACCATCGAGGGCCTGCTCGATGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein comprises a nucleic acid sequence having at least 90% identity (e.g., at least 95% or 100% identity) to the amino acid sequence:

(SEQ ID NO: 74)
ATGTCTAGATTAGATAAAAGTAAAGTGATTAACAGCGCATTAGAGCTGC
TTAATGAGGTCGGAATCGAAGGTTTAGCAACCCGTAAACTCGCCCAGAA
GCTAGGTGTAGAGCAGCCTACATTGTATTGGCATGTAAAAAATAAGCGG
GCTTTGCTCGACGCCTTAGCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGGTGGAGCAAAA
GTACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCATTTTACTTTAGGTTGCGTATTGGAA
GATCAAGAGCATCAAGTCGCTAAAGAAGAAAGGGAAACACCTACTACTG
ATAGTATGCCGCCATTATTACGACAAGCTATCGAATTATTTGATCACCA
AGGTGCAGAGCCAGCCTTCTTATTCGGCCTTGAATTGATCATATGCGGA
TTAGAAAAACAACTTAAATGTGAAAGTGGGTCCGCGTACAGCCGCGCGC
GTACGAAAAACAATTACGGGTCTACCATCGAGGGCCTGCTCGATCTCCC
GGACGACGACGCCCCCGAAGAGGCGGGGCTGGCGGCTCCGCGCCTGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein comprises a nucleic acid sequence having at least 90% identity (e.g., at least 95% or 100% identity) to the amino acid sequence:

(SEQ ID NO: 75)
ATGTCTAGATTAGATAAAAGTAAAGTGATTAACAGCGCATTAGAGCTGC
TTAATGAGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTAGCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGATAGAGCAAAA
GTACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCATTTTACTTTAGGTTGCGTATTGGAA
GATCAAGAGCATCAAGTCGCTAAAGAAGAAAGGGAAACACCTACTACTG
ATAGTATGCCGCCATTATTACGACAAGCTATCGAATTATTTGATCACCA
AGGTGCAGAGCCAGCCTTCTTATTCGGCCTTGAATTGATCATATGCGGA
TTAGAAAAACAACTTAAATGTGAAAGTGGGTCCGCGTACAGCCGCGCGC
GTACGAAAAACAATTACGGGTCTACCATCGAGGGCCTGCTCGATCTCCC
GGACGACGACGCCCCCGAAGAGGCGGGGCTGGCGGCTCCGCGCCTGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein comprises a nucleic acid sequence having at least 90% identity (e.g., at least 95% or 100% identity) to the amino acid sequence:

(SEQ ID NO: 76)
ATGTCTAGATTAGATAAAAGTAAAGTGATTAACAGCGCATTAGAGCTGC
TTAATGAGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTAGCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGATGGAGCAAAA
GAACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCATTTTACTTTAGGTTGCGTATTGGAA
GATCAAGAGCATCAAGTCGCTAAAGAAGAAAGGGAAACACCTACTACTG
ATAGTATGCCGCCATTATTACGACAAGCTATCGAATTATTTGATCACCA
AGGTGCAGAGCCAGCCTTCTTATTCGGCCTTGAATTGATCATATGCGGA
TTAGAAAAACAACTTAAATGTGAAAGTGGGTCCGCGTACAGCCGCGCGC
GTACGAAAAACAATTACGGGTCTACCATCGAGGGCCTGCTCGATCTCCC
GGACGACGACGCCCCCGAAGAGGCGGGGCTGGCGGCTCCGCGCCTGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein comprises a nucleic acid sequence having at least 90% identity (e.g., at least 95% or 100% identity) to the amino acid sequence:

(SEQ ID NO: 77)
ATGTCTAGATTAGATAAAAGTAAAGTGATTAACAGCGCATTAGAGCTGC
TTAATGGGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTAGCCATTGAGATGTTAGATAGGCACCATACTC
ACTTTTGCCCTTTAGAAGGGGAAAGCTGGCAAGATTTTTTACGTAATAA
CGCTAAAAGTTTTAGTGCTTTACTAAGTCATCGCGATGGAGCAAAAGTA
CATTTAGGTACACGGCCTACAGAAAAACAGTATGAAACTCTCGAAAATC
AATTAGCCTTTTTATGCCAACAAGGTTTTTCACTAGAGAATGCATTATA
TGCACTCAGCGCTGTGGGGCATTTTACTTTAGGTTGCGTATTGGAAGAT
CAAGAGCATCAAGTCGCTAAAGAAGAAAGGGAAACACCTACTACTGATA
GTATGCCGCCATTATTACGACAAGCTATCGAATTATTTGATCACCAAGG
TGCAGAGCCAGCCTTCTTATTCGGCCTTGAATTGATCATATGCGGATTA
GAAAAACAACTTAAATGTGAAAGTGGGTCCGCGTACAGCCGCGCGCGTA
CGAAAAACAATTACGGGTCTACCATCGAGGGCCTGCTCGATCTCCCGGA
CGACGACGCCCCCGAAGAGGCGGGGCTGGCGGCTCCGCGCCTGTCCTTT
CTCCCCGCGGGACACACGCGCAGACTGTCGACGGCCCCCCCGACCGATG
TCAGCCTGGGGGACGAGCTCCACTTAGACGGCGAGGACGTGGCGATGGC
GCATGCCGACGCGCTAGACGATTTCGATCTGGACATGTTGGGGGACGGG
GATTCCCCGGGTCCGGGATTTACCCCCCACGACTCCGCCCCCTACGGCG
CTCTGGATATGGCCGACTTCGAGTTTGAGCAGATGTTTACCGATGCCCT
TGGAATTGACGAGTACGGTGGGTAG.

In some embodiments, a Tet responsive activator protein comprises a nucleic acid sequence having at least 90% identity (e.g., at least 95% or 100% identity) to the amino acid sequence:

(SEQ ID NO: 78)
ATGTCTAGATTAGATAAAAGTAAAGTGATTAACAGCGCATTAGAGCTGC
TTAATGAGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTAGCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGATGGAGCAAAA
GAACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCATTTTACTTTAGGTTGCGTATTGGAA
GATCAAGAGCATCAAGTCGCTAAAGAAGAAAGGGAAACACCTACTACTG
ATAGTATGCCGCCATTATTACGACAAGCTATCGAATTATTTGATCACCA
AGGTGCAGAGCCAGCCTTCTTATTCGGCCTTGAATTGATCATATGCGGA
TTAGAAAAACAACTTAAATGTAAAAGTGGGTCCGCGTACAGCCGCGCGC
GTACGAAAAACAATTACGGGTCTACCATCGAGGGCCTGCTCGATCTCCC
GGACGACGACGCCCCCGAAGAGGCGGGGCTGGCGGCTCCGCGCCTGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein comprises a nucleic acid sequence having at least 90% identity (e.g., at least 95% or 100% identity) to the amino acid sequence:

(SEQ ID NO: 78)
ATGTCTAGATTAGATAAAAGTAAAGTGATTAACAGCGCATTAGAGCTGC
TTAATGAGGTCGGAATCGAAGGTTTAACAACCCGTAAACTCGCCCAGAA
GCTAGGTGTAGAGCAGCCTACATTGTATTGGCATGTAAAAAATAAGCGG
GCTTTGCTCGACGCCTTAGCCATTGAGATGTTAGATAGGCACCATACTC
ACTTTTGCCCTTTAGAAGGGGAAAGCTGGCAAGATTTTTTACGTAATAA
CGCTAAAAGTTTTAGATGTGCTTTACTAAGTCATCGCGATGGAGCAAAA
GAACATTTAGGTACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCATTTTACTTTAGGTTGCGTATTGGAA
GATCAAGAGCATCAAGTCGCTAAAGAAGAAAGGGAAACACCTACTACTG
ATAGTATGCCGCCATTATTACGACAAGCTATCGAATTATTTGATCACCA
AGGTGCAGAGCCAGCCTTCTTATTCGGCCTTGAATTGATCATATGCGGA
TTAGAAAAACAACTTAAATGTAAAAGTGGGTCCGCGTACAGCCGCGCGC
GTACGAAAAACAATTACGGGTCTACCATCGAGGGCCTGCTCGATCTCCC
GGACGACGACGCCCCCGAAGAGGCGGGGCTGGCGGCTCCGCGCCTGTCC
TTTCTCCCCGCGGGACACACGCGCAGACTGTCGACGGCCCCCCCGACCG
ATGTCAGCCTGGGGGACGAGCTCCACTTAGACGGCGAGGACGTGGCGAT
GGCGCATGCCGACGCGCTAGACGATTTCGATCTGGACATGTTGGGGGAC
GGGGATTCCCCGGGTCCGGGATTTACCCCCCACGACTCCGCCCCCTACG
GCGCTCTGGATATGGCCGACTTCGAGTTTGAGCAGATGTTTACCGATGC
CCTTGGAATTGACGAGTACGGTGGGTAG.

In some embodiments, a Tet responsive activator protein is a variant of a Tet responsive activator protein and comprises the sequence set forth in SEQ ID NO:40 but with the following amino acid substitutions: 86Y A209T; V9I F86Y A209T; F67S F86Y A209T; G138D F86Y A209T; E157K F86Y A209T; R171K F86Y A209T; V9I G138D F86Y A209T; V9I E157K F86Y A209T; V9I R171K F86Y A209T; F67S R171K F86Y A209T; V9I F67S F86Y A209T; F67S G138D F86Y A209T; F67S E157K F86Y A209T; V9I F67S G138D F86Y A209T; V9I F67S E157K F86Y A209T; V9I F67S R171K F86Y A209T; V9I G138D E157K F86Y A209T; V9I G138D R171K F86Y A209T; F86Y; F86Y A209T; F67S F86Y A209T; G138d F86Y A209T; E157K F86Y A209T; R171K F86Y A209T; V9I G138D F86Y A209T; V9I E157K F86Y A209T; V9I R171K F86Y A209T; F177L F86Y A209T; F67S F177L F86Y A209T; C195S F86Y A209T; G138S F86Y A209T; C68R F86Y A209T; V9I F67S F86Y A209T; F67S G138D F86Y A209T; F67S E157K F86Y A209T; F67S R171K F86Y A209T; V9I F67S G138D F86Y A209T; V9I F67S E157K F86Y A209T; V9I F67S R171K F86Y A209T; V9I G138D E157K F86Y A209T; V9I G138D R171K F86Y A209T; S12G F67S F86Y A209T; G19M F67S F86Y A209T; E37Q F67S F86Y A209T; C68R G138D F86Y A209T; G19M G138D F86Y A209T; E37Q G138D F86Y A209T; V9I C68R G138D F86Y A209T; V9I G19M G138D F86Y A209T; V9I E37Q G138D F86Y A209T; F67S; G138D; E157K; R171K; V9I G138D; V9I E157K; V9I R171K; F177L; F67S F177L; C195S; G138S; C68R; V9I F67S; F67S G138D; F67S E157K; F67S R171K; V9I F67S G138D; V9I F67S E157K; V9I F67S R171K; V9I G138D E157K; V9I G138D R171K; S12G F67S; G19M F67S; E37Q F67S; V9I C68R G138D; V9I G19M G138D; V9I E37Q G138D; V9I G19M F67S G138D; V9I S12G F67S G138D; V9I F67S C68R G138D; F67S F86Y; G138D F86Y; E157K F86Y; R171K F86Y; V9I G138D F86Y; V9I E157K F86Y; V9I R171K F86Y; F177L F86Y; F67S F177L F86Y; C195S F86Y; G138S F86Y; C68R F86Y; V9I F67S F86Y; F67S G138D F86Y; F67S E157K F86Y; F67S R171K F86Y; V9I F67S G138D F86Y; V9I F67S E157K F86Y; V9I F67S R171K F86Y; V9I G138D E157K F86Y; V9I G138D R171K F86Y; S12G F67S F86Y; G19M F67S F86Y; E37Q F67S F86Y; V9I C68R G138D F86Y; V9I G19M G138D F86Y; V9I E37Q G138D F86Y; V9I G19M F67S G138D F86Y; V9I S12G F67S G138D F86Y; V9I F67S C68R G138D F86Y; F67S A209T; G138D A209T; E157K A209T; R171K A209T; V9I G138D A209T; V9I E157K A209T; V9I R171K A209T; F177L A209T; F67S F177L A209T; C195S A209T; G138S A209T; C68R A209T; V9I F67S A209T; F67S G138D A209T; F67S E157K A209T; F67S R171K A209T; V9I F67S G138D A209T; V9I F67S E157K A209T; V9I F67S R171K A209T; V9I G138D E157K A209T; V9I G138D R171K A209T; S12G F67S A209T; G19M F67S A209T; E37Q F67S A209T; V9I C68R G138D A209T; V9I G19M G138D A209T; V9I E37Q G138D A209T; V9I G19M F67S G138D A209T; V9I S12G F67S G138D A209T; V9I F67S C68R G138D A209T; G19M F67S V9I G138D F86Y A209T; S12G F67S V9I G138D F86Y A209T; or C68R F67S V9I G138D F86Y A209T; where the numbering of the substituted amino acids is based on the numbering of amino acids in SEQ ID NO:40.

In some embodiments, a Tet responsive activator protein comprises an amino acid sequence having at least 90% identity to the amino acid sequence:

(SEQ ID NO: 41)
MSRLDKSKVINSALELLNGVGIEGLTTRKLAQKLGVEQPTLYWHVKNKR
ALLDALPIEMLDRHHTHFCPLEGESWQDFLRNNAKSFRCALLSHRDGAK
VHLGTRPTEKQYETLENQLAFLCQQGFSLENALYALSAVGHFTLGCVLE
EQEHQVAKEERETPTTDSMPPLLRQAIELFDRQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 42)
MSRLDKSKVINSALELLNGVGIEGLTTRKLAQKLGVEQPTLYWHVKNKR
ALLDALPIEMLDRHHTHFCPLEGESWQDFLRNNAKSFRCALLSHRDGAK
VHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 43)
MSRLDKSKVINGALELLNGVGIEGLTTRKLAQKLGVEQPTLYWHVKNKR
ALLDALPIEMLDRHHTHFCPLEGESWQDFLRNNAKSFRCALLSHRDGAK
VHLGTRPTEKQYETLENQLAFLCQQGFSLENALYALSAVGHFTLGCVLE
EQEHQVAKEERETPTTDSMPPLLRQAIELFDRQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 44)
MSRLDKSKVINSALELLNEVGIEGLATRKLAQKLGVEQPTLYWHVKNKR
ALLDALAIEMLDRHHTHFCPLEGESWQDFLRNNAKSFRCALLSHRGGAK
VHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 45)
MSRLDKSKVINSALELLNEVGIEGLTTRKLAQKLGVEQPTLYWHVKNKR
ALLDALAIEMLDRHHTHFCPLEGESWQDFLRNNAKSFRCALLSHRDRAK
VHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 80)
MSRLDKSKVINSALELLNEVGIEGLTTRKLAQKLGVEQPTLYWHVKNKR
ALLDALAIEMLDRHHTHFCPLEGESWQDFLRNNAKSFRCALLSHRDGAK
EHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 81)
MSRLDKSKVINSALELLNGVGIEGLTTRKLAQKLGVEQPTLYWHVKNKR
ALLDALAIEMLDRHHTHFCPLEGESWQDFLRNNAKSFSALLSHRDGAKV
HLGTRPTEKQYETLENQLAFLCQQGFSLENALYALSAVGHFTLGCVLED
QEHQVAKEERETPTTDSMPPLLRQAIELFDHQGAEPAFLFGLELIICGL
EKQLKCESGSAYSRARTKNNYGSTIEGLLDLPDDDAPEEAGLAAPRLSF
LPAGHTRRLSTAPPTDVSLGDELHLDGEDVAMAHADALDDFDLDMLGDG
DSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 82)
MSRLDKSKVINSALELLNEVGIEGLTTRKLAQKLGVEQPTLYWHVKNKR
ALLDALAIEMLDRHHTHFCPLEGESWQDFLRNNAKSFRCALLSHRDGAK
EHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCKSGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 83)
MSRLDKSKVINSALELLNEVGIEGLTTRKLAQKLGVEQPTLYWHVKNKR
ALLDALAIEMLDRHHTHFCPLKGESWQDFLRNNAKSFRCALLSHRNGAK
VHSDTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 84)
MSRLDKSKVINGALELLNGVGIEGLTTRKLAQKLGVEQPTLYWHVKNKR
ALLDALPIEMLDRHHTHFCPLEGESWQDFLRNNAKSFRCALLSHRDGAK
VHLGTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a Tet responsive activator protein comprises an amino acid sequence having at least 90% identity to the amino acid sequence:

(SEQ ID NO: 43)
MSRLDKSKVINGALELLNGVGIEGLTTRKLAQKLGVEQPTLYWHVKNKR
ALLDALPIEMLDRHHTHFCPLEGESWQDFLRNNAKSFRCALLSHRDGAK
VHLGTRPTEKQYETLENQLAFLCQQGFSLENALYALSAVGHFTLGCVLE
EQEHQVAKEERETPTTDSMPPLLRQAIELFDRQGAEPAFLFGLELIICG
LEKQLKCESGSAYSRARTKNNYGSTIEGLLDLPDDDAPEEAGLAAPRLS
FLPAGHTRRLSTAPPTDVSLGDELHLDGEDVAMAHADALDDFDLDMLGD
GDSPGPGFTPHDSAPYGALDMADFEFEQMFTDALGIDEYGG.

In some embodiments, a TRE comprises a nucleotide sequence having at least 80% identity (e.g., at least 85%, at least 90%, at least 95%, or a 100% identity) to the nucleotide sequence:

(SEQ ID NO: 46)
GAATTCCTCGAGTTTACCACTCCCTATCAGTGATAGAGAAAAGTGAAAG
TCGAGTTTACCACTCCCTATCAGTGATAGAGAAAAGTGAAAGTCGAGTT
TACCACTCCCTATCAGTGATAGAGAAAAGTGAAAGTCGAGTTTACCACT
CCCTATCAGTGATAGAGAAAAGTGAAAGTCGAGTTTACCACTCCCTATC
AGTGATAGAGAAAAGTGAAAGTCGAGTTTACCACTCCCTATCAGTGATA
GAGAAAAGTGAAAGTCGAGTTTACCACTCCCTATCAGTGATAGAGAAAA
GTGAAAGTCGAGCTCGGTACCCGGGTCGAGTAGGCGTGTACGGTGGGAG
GCCTATATAAGCAGAGCTCGTTTAGTGAACCGTCAGATCGCCTGGAGAC
GCCATCCACGCTGTTTTGACCTCCATAGAAGACACCGGGACCGATCCAG
CCTCCGCGG.

In some embodiments, a TRE comprises a nucleotide sequence having at least 80% identity (e.g., at least 85%, at least 90%, at least 95%, or a 100% identity) to the nucleotide sequence:

(SEQ ID NO: 47)
GAATTCCTCGACCCGGGTACCGAGCTCGACTTTCACTTTTCTCTATCAC
TGATAGGGAGTGGTAAACTCGACTTTCACTTTTCTCTATCACTGATAGG
GAGTGGTAAACTCGACTTTCACTTTTCTCTATCACTGATAGGGAGTGGT
AAACTCGACTTTCACTTTTCTCTATCACTGATAGGGAGTGGTAAACTCG
ACTTTCACTTTTCTCTATCACTGATAGGGAGTGGTAAACTCGACTTTCA
CTTTTCTCTATCACTGATAGGGAGTGGTAAACTCGACTTTCACTTTTCT
CTATCACTGATAGGGAGTGGTAAACTCGAGTAGGCGTGTACGGTGGGAG
GCCTATATAAGCAGAGCTCGTTTAGTGAACCGTCAGATCGCCTGGAGAC
GCCATCCACGCTGTTTTGACCTCCATAGAAGACACCGGGACCGATCCAG
CCTCCGCGG.

In some embodiments, a TRE comprises a nucleotide sequence having at least 80% identity (e.g., at least 85%, at least 90%, at least 95%, or a 100% identity) to the nucleotide sequence:

(SEQ ID NO: 48)
GAGCTCGACTTTCACTTTTCTCTATCACTGATAGGGAGTGGTAAACTCG
ACTTTCACTTTTCTCTATCACTGATAGGGAGTGGTAAACTCGACTTTCA
CTTTTCTCTATCACTGATAGGGAGTGGTAAACTCGACTTTCACTTTTCT
CTATCACTGATAGGGAGTGGTAAACTCGACTTTCACTTTTCTCTATCAC
TGATAGGGAGTGGTAAACTCGACTTTCACTTTTCTCTATCACTGATAGG
GAGTGGTAAACTCGACTTTCACTTTTCTCTATCACTGATAGGGAGTGGT
AAACTCGAGATCCGGCGAATTCGAACACGCAGATGCAGTCGGGGCGGCG
CGGTCCGAGGTCCACTTCGCATATTAAGGTGACGCGTGTGGCCTCGAAC
ACCGAG.

In some embodiments, a TRE comprises a nucleotide sequence having at least 80% identity (e.g., at least 85%, at least 90%, at least 95%, or a 100% identity) to the nucleotide sequence:

(SEQ ID NO: 50)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTTAGGCGTGTACGGTGGGCGCCTATAAAAGCAGAGCTCGTTTA
GTGAACCGTCAGATCGCCTGGAGACGCCATCCACGCTGTTTCCATAGAA
GA.

In some embodiments, a TRE comprises a nucleotide sequence having at least 80% identity (e.g., at least 85%, at least 90%, at least 95%, or a 100% identity) to the nucleotide sequence:

(SEQ ID NO: 51)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTTAGGCGTGTACGGTGGGCGCCTATAAAAGCAGAGCTCGTTTA
GTGAACCGTCAGATCGCCTGGAGA.

In some embodiments, a TRE comprises a nucleotide sequence having at least 80% identity (e.g., at least 85%, at least 90%, at least 95%, or a 100% identity) to the nucleotide sequence:

(SEQ ID NO: 52)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTTAGGCGTGTACGGTGGGCGCCTATAAAAGCAGAGCTCGTTTA
GTGAACCGTCAGATCGCCTGGAGGTAATCAACTACCAATTCCAGCTCTC
TTTTGACAACTGGTCTTATACCAACTTTCCGTACCACTTCCTACCCTCG
TAAGACAATTGCAA.

In some embodiments, a TRE comprises a nucleotide sequence having at least 80% identity (e.g., at least 85%, at least 90%, at least 95%, or a 100% identity) to the nucleotide sequence:

(SEQ ID NO: 53)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTTAGGCGTGTACGGTGGGCGCCTATAAAAGCAGAGCTCGTTTA
GTGAACCGTCAGATCGCCTGGAGCAATTCCACAACACTTTTGTCTTATA
CCAACTTTCCGTACCACTTCCTACCCTCGTAAA.

In some embodiments, a TRE comprises a nucleotide sequence having at least 80% identity (e.g., at least 85%, at least 90%, at least 95%, or a 100% identity) to the nucleotide sequence:

(SEQ ID NO: 54)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTTAGGCGTGTACGGTGGGCGCCTATAAAAGCAGAGCTCGTTTA
GTGAACCGTCAGATCGCCTGGAGCTAATCAACTACCAATTCCAGCTCTC
TTTTGACAACTGGTCTTATACCAACTTTCCGTACCACTTCCTACCCTCC
TAAGACAATTGCAAA.

In some embodiments, a TRE comprises a nucleotide sequence having at least 80% identity (e.g., at least 85%, at least 90%, at least 95%, or a 100% identity) to the nucleotide sequence:

(SEQ ID NO: 55)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTGCCTATGTTCTTTTGGAATCTATCCAAGTCTTATGTAAATGC
TTATGTAAACCATAATATAAAAGAGTGCTGATTTTTTGAGTAAACTTGC
AACAGTCCTAACATTCTTCTCTCGTGTGTTTGTGTCTGTTCGCCATCCC
GTCTCCGCTCGTCACTTATCCTTCACTTTTCAGAGGGTCCCCCCGCAGA
TCCCGGTCACCCTCAGGTCGG.

In some embodiments, a TRE comprises a nucleotide sequence having at least 80% identity (e.g., at least 85%, at least 90%, at least 95%, or a 100% identity) to the nucleotide sequence:

(SEQ ID NO: 56)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTCCATAATATAAAAGAGTGCTGATTTTTTGAGTAAACTTGCAA
CAGTCCTAACATTCTTCTCTCGTGTGTTTGTGTCTGTTCGCCATCCCGT
CTCCGCTCGTCACTTATCCTTCACTTTTCAGAGGGTCCCCCCGCAGATC
CCGGTCACCCTCAGGTCGG.

In some embodiments, a TRE comprises a nucleotide sequence having at least 80% identity (e.g., at least 85%, at least 90%, at least 95%, or a 100% identity) to the nucleotide sequence:

(SEQ ID NO: 57)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTCCAGGGCGCCTATAAAAGAGTGCTGATTTTTTGAGTAAACTT
GCAACAGTCCTAACATTCTTCTCTCGTGTGTTTGTGTCTGTTCGCCATC
CCGTCTCCGCTCGTCACTTATCCTTCACTTTTCAGAGGGTCCCCCCGCA
GATCCCGGTCACCCTCAGGTCGG.

In some embodiments, a TRE comprises a nucleotide sequence having at least 80% identity (e.g., at least 85%, at least 90%, at least 95%, or a 100% identity) to the nucleotide sequence:

(SEQ ID NO: 58)
TTTACTCCCTATCAGTGATAGAGAACGTATGAAGAGTTTACTCCCTATC
AGTGATAGAGAACGTATGCAGACTTTACTCCCTATCAGTGATAGAGAAC
GTATAAGGAGTTTACTCCCTATCAGTGATAGAGAACGTATGACCAGTTT
ACTCCCTATCAGTGATAGAGAACGTATCTACAGTTTACTCCCTATCAGT
GATAGAGAACGTATATCCAGTTTACTCCCTATCAGTGATAGAGAACGTA
TAAGCTTTGCTTATGTAAACCAGGGCGCCTATAAAAGAGTGCTGATTTT
TTGAGTAAACTTCAATTCCACAACACTTTTGTCTTATACCAACTTTCCG
TACCACTTCCTACCCTCGTAAA.

In some embodiments, a TRE comprises a nucleotide sequence having at least 80% identity (e.g., at least 85%, at least 90%, at least 95%, or a 100% identity) to the nucleotide sequence:

(SEQ ID NO: 59)
GAATTCTTTACTCCCTATCAGTGATAGAGAATGTATGAAGAGTTTACTC
CCTATCAGTGATAGAGAATGTATGCAGACTTTACTCCCTATCAGTGATA
GAGAATGTATAAGGAGTTTACTCCCTATCAGTGATAGAGAATGTATGAC
CAGTTTACTCCCTATCAGTGATAGAGAATGTATCTACAGTTTACTCCCT
ATCAGTGATAGAGAATGTATATCCAGTTTACTCCCTATCAGTGATAGAG
AATGTATAAGCTTTAGG.

In some embodiments, a TRE comprises a nucleotide sequence having at least 80% identity (e.g., at least 85%, at least 90%, at least 95%, or a 100% identity) to the nucleotide sequence:

(SEQ ID NO: 60)
CATGTACAGTGGGCACCTATAAAAGCAGAGCTCATTTAGTGAACTGTCA
GATTGCCTGGAGCAATTCCACAACACTTTTGTCTTATACCAACTTTCCA
TACCACTTCCTACCCTCATAAAGTGCACACCATGG.

In some embodiments, a TRE comprises a nucleotide sequence having at least 80% identity (e.g., at least 85%, at least 90%, at least 95%, or a 100% identity) to the nucleotide sequence:

(SEQ ID NO: 61)
CCATGGTGTGCACTTTATGAGGGTAGGAAGTGGTATGGAAAGTTGGTAT
AAGACAAAAGTGTTGTGGAATTGCTCCAGGCAATCTGACAGTTCACTAA
ATGAGCTCTGCTTTTATAGGTGCCCACTGTACATGCCTAAGAATTCTTT
ACT.

In some embodiments, a TRE comprises a nucleotide sequence having at least 80% identity (e.g., at least 85%, at least 90%, at least 95%, or a 100% identity) to the nucleotide sequence:

(SEQ ID NO: 62)
CCCTATCAGTGATAGAGAATGTATGAAGAGTTTACTCCCTATCAGTGAT
AGAGAATGTATGCAGACTTTACTCCCTATCAGTGATAGAGAATGTATAA
GGAGTTTACTCCCTATCAGTGATAGAGAATGTATGACCAGTTTACTCCC
TATCAGTGATAGAGAATGTATCTACAGTTTACTCCCTATCAGTGATAGA
GAATGTATATCCAGTTTACTCCCTATCAGTGATAGAGAATGTATAAGCT
TTAGG.

In some embodiments, a minimal promoter of a TRE comprises a nucleotide sequence having at least 80% identity (e.g., at least 85%, at least 90%, at least 95%, or a 100% identity) to the nucleotide sequence:

(SEQ ID NO: 63)
GGTAGGCGTGTACGGTGGGAGGCCTATATAAGCAGAGCTCGTTTAGTGA
ACCGTCAGATCGCCTGGAGACGCCATCCACGCTGTTTTGACCTCCATAG
AAGACACCGGGACCGATCCAGCCTCCGCGG.

In some embodiments, a minimal promoter of a TRE comprises a nucleotide sequence having at least 80% identity (e.g., at least 85%, at least 90%, at least 95%, or a 100% identity) to the nucleotide sequence:

(SEQ ID NO: 64)
GGTAGGCGTGTACGGTGGGCGCCTATAAAAGCAGAGCTCGTTTAGTGAA
CCGTCAGATCGCCTGGAGACGCCATCCACGCTGTTTTGACCTCCATAGA
AGACACCGGGACCGATCCAGCCTCCGCG.

In some embodiments, a minimal promoter of a TRE comprises a nucleotide sequence having at least 80% identity (e.g., at least 85%, at least 90%, at least 95%, or a 100% identity) to the nucleotide sequence:

(SEQ ID NO: 65)
TAGGCGTGTACGGTGGGCGCCTATAAAAGCAGAGCTCGTTTAGTGAAC
CGTCAGATCGCCTGGAGA.

In some embodiments, a minimal promoter of a TRE comprises a nucleotide sequence having at least 80% identity (e.g., at least 85%, at least 90%, at least 95%, or a 100% identity) to the nucleotide sequence:

(SEQ ID NO: 66)
GTAATCAACTACCAATTCCAGCTCTCTTTTGACAACTGGTCTTATACCA
ACTTTCCGTACCACTTGCAACCCTCGTAAGACAATTGCAA.

In some embodiments, a minimal promoter of a TRE comprises a nucleotide sequence having at least 80% identity (e.g., at least 85%, at least 90%, at least 95%, or a 100% identity) to the nucleotide sequence:

(SEQ ID NO: 67)
AATTCCACAACACTTTTGTCTTATACCAACTTTCCGTACCACTTCCTAC
CCTCGTAAA.

In some embodiments, a minimal promoter of a TRE comprises a nucleotide sequence having at least 80% identity (e.g., at least 85%, at least 90%, at least 95%, or a 100% identity) to the nucleotide sequence:

(SEQ ID NO: 68)
GCCTATGTTCTTTTGGAATCTATCCAAGTCTTATGTAAATGCTTATGTA
AACCATAATATAAAAGAGTGCTGATTTTTTGAGTAAACTTGCAACAGTC
CTAACATTCTTCTCTCGTGTGTTTGTGTCTGTTCGCCATCCCGTCTCCG
CTCGTCACTTATCCTTCACTTTTCAGAGGGTCCCCCCGCAGATCCCGGT
CACCCTCAGGTCGG.

In some embodiments, a Tet Repressor binding protein may comprise a sequence having at least 90% identity (e.g., at least 95% or 100% identity) to the amino acid sequence:

(SEQ ID NO: 49)
MSRLDKSKVINSALELLNEVGIEGLTTRKLAQKLGVEQPTLYWHVKNKR
ALLDALAIEMLDRHHTHFCPLKGESWQDFLRNKAKSFRCALLSHRNGAK
VHSDTRPTEKQYETLENQLAFLCQQGFSLENALYALSAVGHFTLGCVLE
DQEHQVAKEERETPTTDSMPPLLRQAIELFDHQGAEPAFLFGLELIICG
LEKQLKCESGS.

In some embodiments, a Tet Repressor binding protein may comprise a sequence having at least 90% identity (e.g., at least 95% or 100% identity) to the amino acid sequence:

(SEQ ID NO: 86)
ATGTCTAGATTAGATAAAAGTAAAGTGATTAACAGCGCATTAGAGCTGC
TTAATGAGGTCGGAATCGAAGGTTTAACAACCCGTAAACTCGCCCAGAA
GCTAGGTGTAGAGCAGCCTACACTGTATTGGCATGTAAAAAATAAGCGG
GCTTTGCTCGACGCCTTAGCCATTGAGATGTTAGATAGGCACCATACTC
ACTTTTGCCCTTTAAAAGGGGAAAGCTGGCAAGATTTTTTACGCAATAA
GGCTAAAAGTTTTAGATGTGCTTTACTAAGTCATCGCAATGGAGCAAAA
GTACATTCAGATACACGGCCTACAGAAAAACAGTATGAAACTCTCGAAA
ATCAATTAGCCTTTTTATGCCAACAAGGTTTTTCACTAGAGAATGCATT
ATATGCACTCAGCGCTGTGGGGCATTTTACTTTAGGTTGCGTATTGGAA
GATCAAGAGCATCAAGTCGCTAAAGAAGAAAGGGAAACACCTACTACTG
ATAGTATGCCGCCATTATTACGACAAGCTATCGAATTATTTGATCACCA
AGGTGCAGAGCCAGCCTTCTTATTCGGCCTTGAATTGATCATATGCGGA
TTAGAAAAACAACTTAAATGTGAAAGTGGGTCC.

In some embodiments, the minimal promoter comprises a sequence having at least 70%, 80%, 90%, 95%, 99%, or 100% sequence identity to any one of SEQ ID NOs: 63-68. In some embodiments, the inducible promoter comprises a sequence having at least 70%, 80%, 90%, 95%, 99%, or 100% sequence identity to any one of SEQ ID NOs: 22, 46-48, or 50-62. In some embodiments, the rTA comprises a sequence having at least 70%, 80%, 90%, 95%, 99%, or 100% sequence identity to any one of SEQ ID NOs: 21, 40-45, or 69-86, or variants thereof.

The Tet responsive activator protein or variant thereof, the Tet Repressor binding protein or variant thereof, TRE sequence, or any tetracycline-inducible promoter sequence or variant thereof can be any of those disclosed in U.S. Pat. Nos. 7,541,446; 8,383,364; 6,136,954; 5,814,618; 6,271,348; 5,789,156; 7,666,668; 6,914,124; 5,650,298; 5,922,927; 5,464,758; 5,866,755; 5,589,362; 5,654,168; 6,242,667; 5,912,411; 6,783,756; 5,888,981; 6,004,941; 6,252,136; 5,859,310; 6,271,341; 6,087,166; US2003022315; US20050037335; U.S. Pat. No. 9,181,556; and WO03056021, which are each herein incorporated by reference in their entirety.

Any of the proteins described herein may be expressed from a nucleotide sequence that has been codon-optimized to increase expression in a host cell, e.g., a mammalian cell or a human cell line.

In other embodiments, an insect gene control element is used to provide mammalian inducible expression. For example, an ecdysone-responsive element and a gene encoding the ecdysone receptor can be included in a construct to allow mammalian expression to be induced by the insect hormone ecdysone or analogs or derivatives thereof, such as ponasterone. In mammalian cells, the ecdysone receptor heterodimerizes with the retinoid X receptor (RXR). The ecdysone-responsive element comprises a binding site for the RXR-ecdysone receptor heterodimer, which is typically a synthetic recognition site for the heterodimer that preferably does not bind any endogenous transcription factors or natural nuclear hormone receptors. In the presence of ecdysone or an analog or derivative thereof, the RXR-ecdysone receptor heterodimer binds to the ecdysone-responsive element to activate transcription from the promoter. For a description of ecdysone-responsive promoters, see, e.g., No et al. (1996) Proc. Natl. Acad. Sci. USA 93 (8): 3346-51, Oehme et al. (2006) Cell Death and Differentiation (2006) 13:189-201; herein incorporated by reference.

In some embodiments, the second construct or an additional separate construct comprises an element responsive to a fourth expression triggering agent. In certain embodiments, the fourth expression triggering agent-responsive element comprises a plurality of hormone-response elements. In particular embodiments, the hormone-response elements are estrogen responsive elements (EREs). In various embodiments, the third expression triggering element is the same as the first expression triggering element, and the fourth expression triggering element is the same as the second expression triggering element.

In some embodiments, the inducible promoter comprises a plurality of Tet operator elements capable of binding to a Tet responsive activator protein in the presence of a third expression triggering agent. In particular embodiments, the third expression triggering agent is the same as the first expression triggering agent.

In some embodiments, the recombinase coding sequence is flanked by a first recombinase site and a second recombinase site. In some embodiments, the recombinase is Cre. In some embodiments, the Cre coding sequence is flanked by a first lox site and a second lox site. In some embodiments, the first polyA sequence is positioned between the Cre coding sequence and adenoviral helper protein coding sequences that encode one or both of adenovirus E2A and E4. The strong 3′ polyadenylation signal positioned upstream (5′ to) the coding sequences for the adenovirus helper proteins prevents basal expression of the downstream adenoviral helper genes, E2A and E4.

In some embodiments, helper construct does not comprise a recombinase that is self-excising. In some embodiments, the recombinase is a site-specific recombinase. In some embodiments, the recombinase is fused to a ligand binding domain. In some embodiments, the recombinase is Cre polypeptide or flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the recombinase translocates to the nucleus in the presence of a triggering agent. In some embodiments, the triggering agent is an estrogen receptor ligand. In some embodiments, the triggering agent is a selective estrogen receptor modulator (SERM). In some embodiments, the triggering agent is tamoxifen. In some embodiments, the recombinase is self-excising to reduce toxicity of the expressed recombinase to cell after induction of expression of the recombinase. In some embodiments, the Cre is self-excising to reduce toxicity of the expressed Cre to cell after induction of expression of the Cre. In some embodiments, the Cre-ERT2 is self-excising to reduce toxicity of the expressed Cre-ERT2 to cell after induction of expression of the Cre-ERT2. When the recombinase is not self-excisable, the sequence or sequences encoding one or more helper proteins can be operably linked to an inducible promoter. When the recombinase is not self-excisable, the sequence or sequences encoding one or more helper proteins can be downstream of an excisable element (e.g., a sequence flanked by recombination sites and comprising a stop signal) and constitutive promoter, wherein upon excision of the excisable element (e.g., by a recombinase), the sequence or sequences encoding one or more helper proteins are operably linked to the constitutive promoter.

In some embodiments, the further segment shown in FIG. 2C provides for inducible production of VA-RNA from construct 2.

In this embodiment, the further segment includes a Cre-inducible U6 or U7 promoter. The U6 or U7 promoter is split into 2 parts separated by a Lox flanked stuffer sequence. The U6 or U7 promoter is inactive because of the presence of the stuffer sequence. Cre mediated excision of the stuffer activates the U6 or U7 promoter. The U6 or U7 promoter drives the expression of transcriptionally dead mutants of VA RNA1 (a preferred embodiment is a double point mutant G16A-G60A). Other embodiments provide for alternative sources of VA-RNA.

In various embodiments, the coding sequence for the first expression triggering agent-responsive protein is operatively linked to a CMV promoter. In some embodiments, the coding sequence for the first expression triggering agent-responsive protein comprises a coding sequence for the Tet responsive activator protein. In particular embodiments, the Tet responsive activator protein is Tet-on-3G activator protein.

In various embodiments, the second mammalian cell selection element confers antibiotic resistance. In particular embodiments, the antibiotic resistance conferring element is a blasticidin resistance gene.

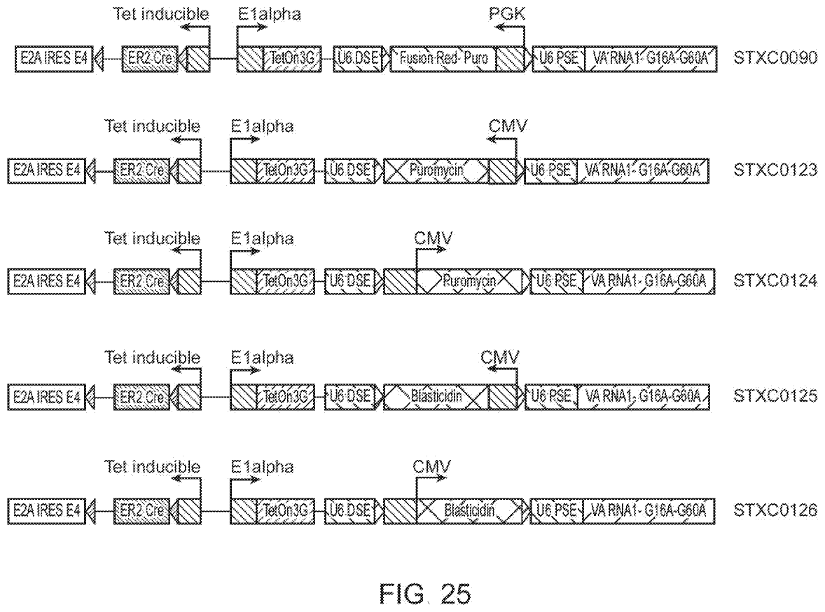

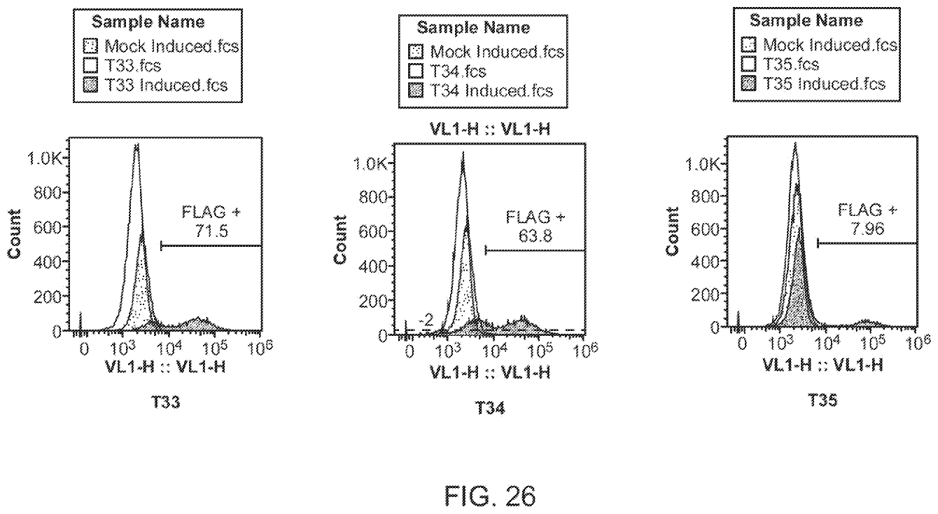

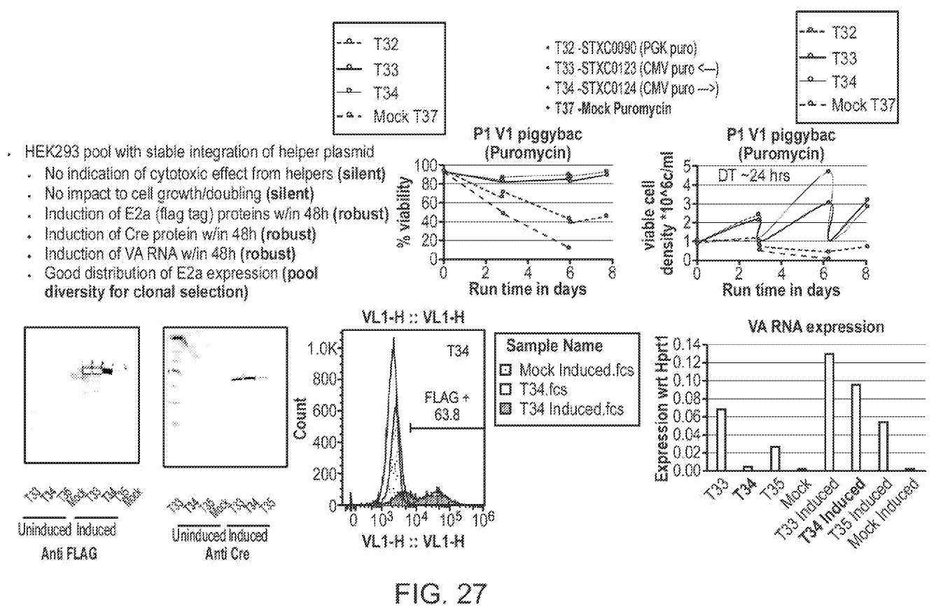

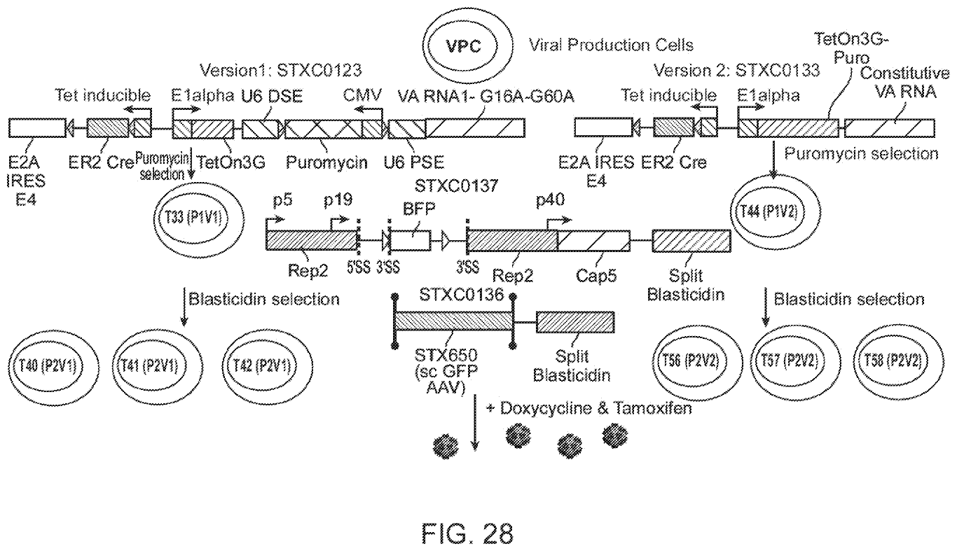

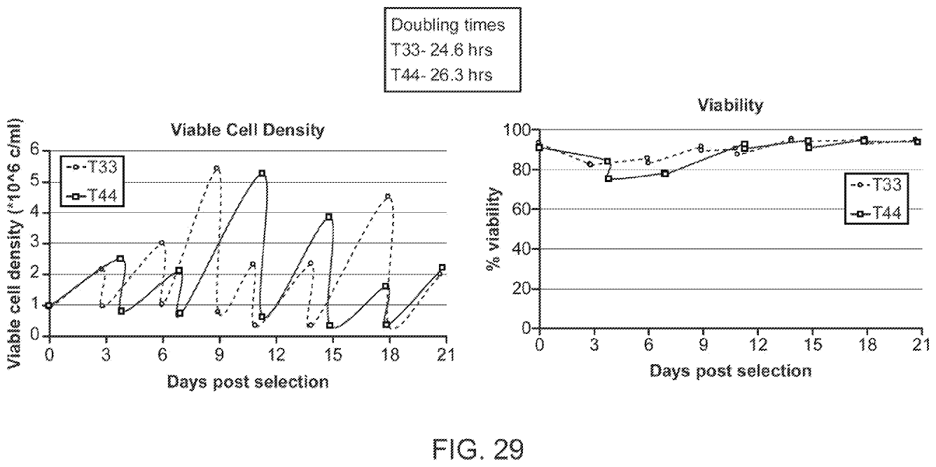

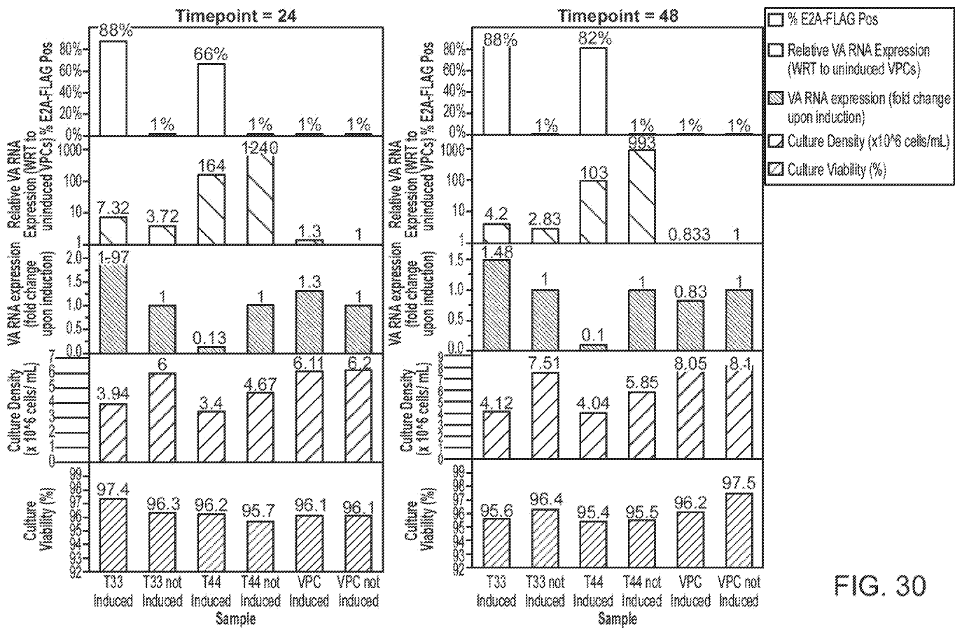

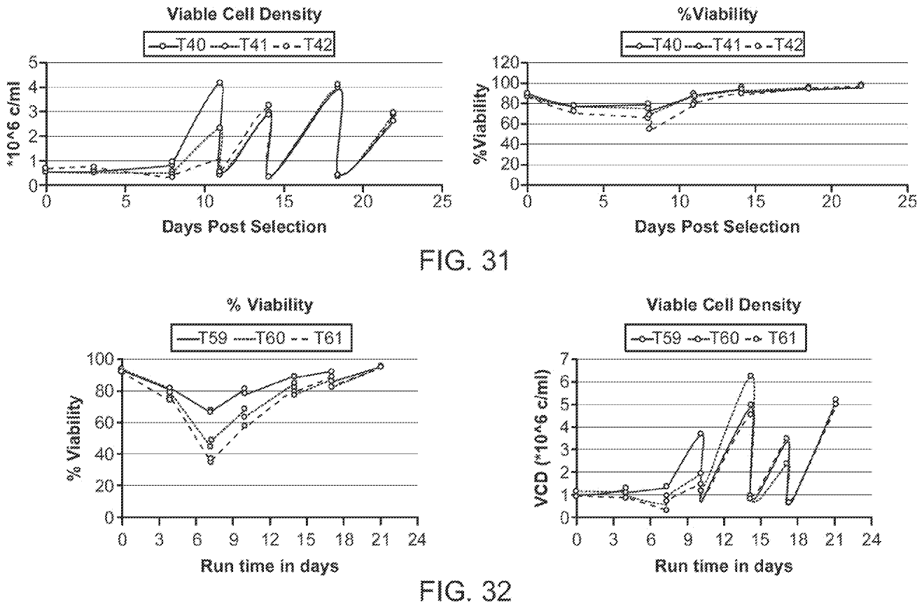

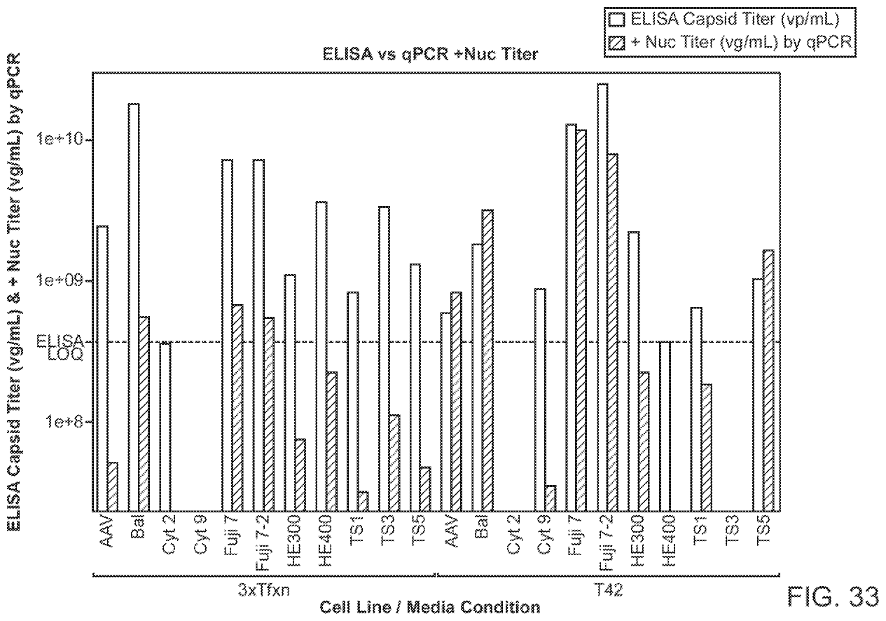

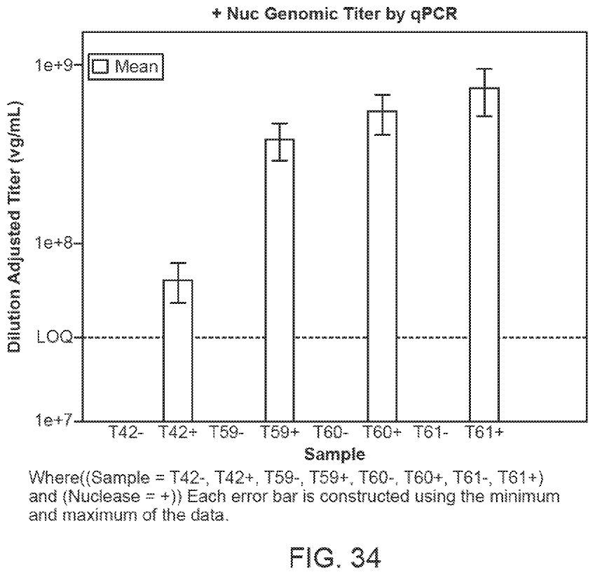

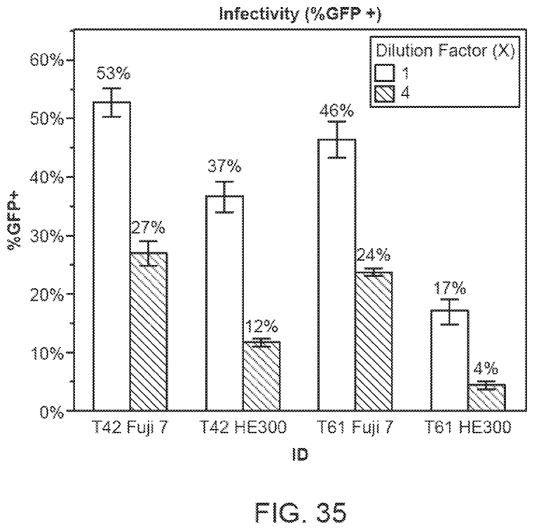

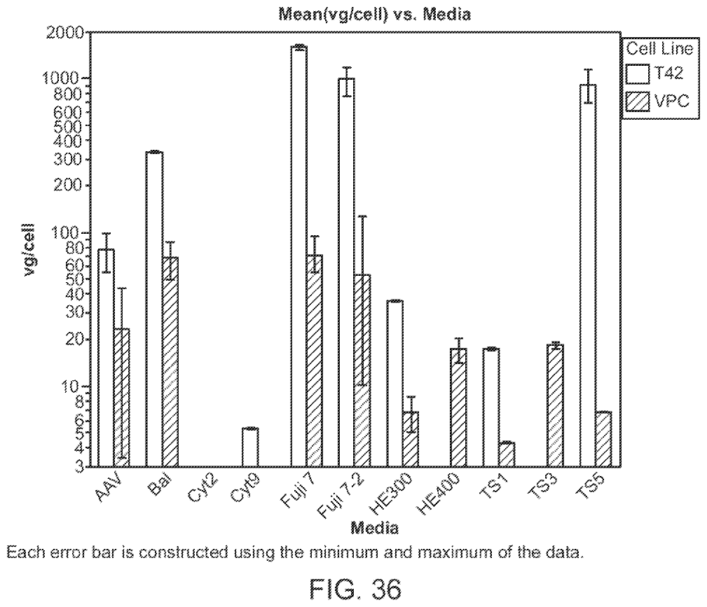

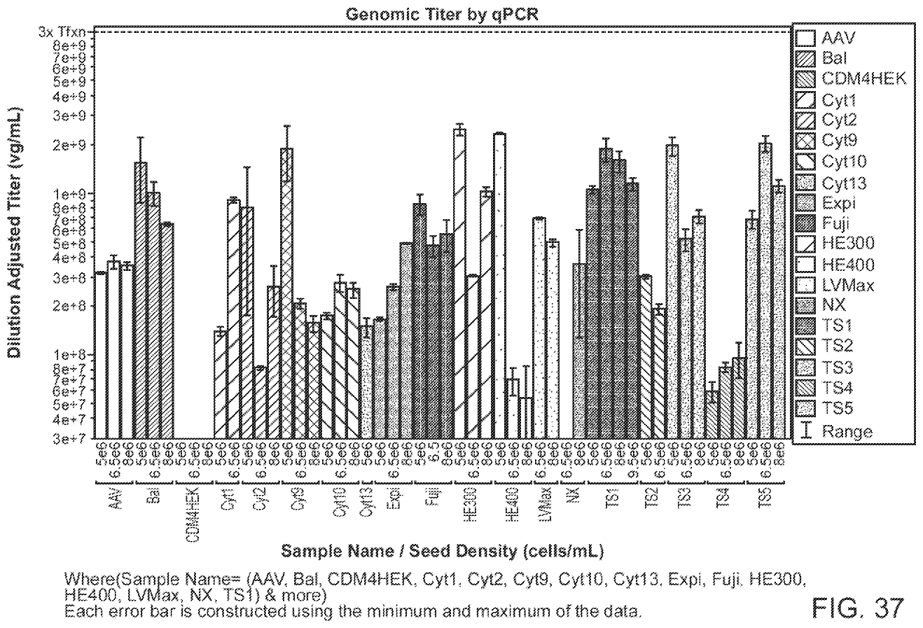

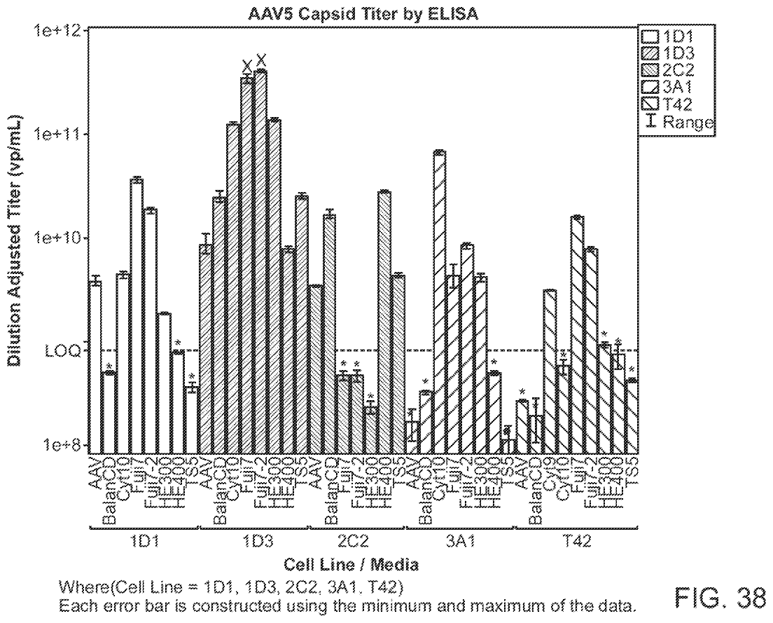

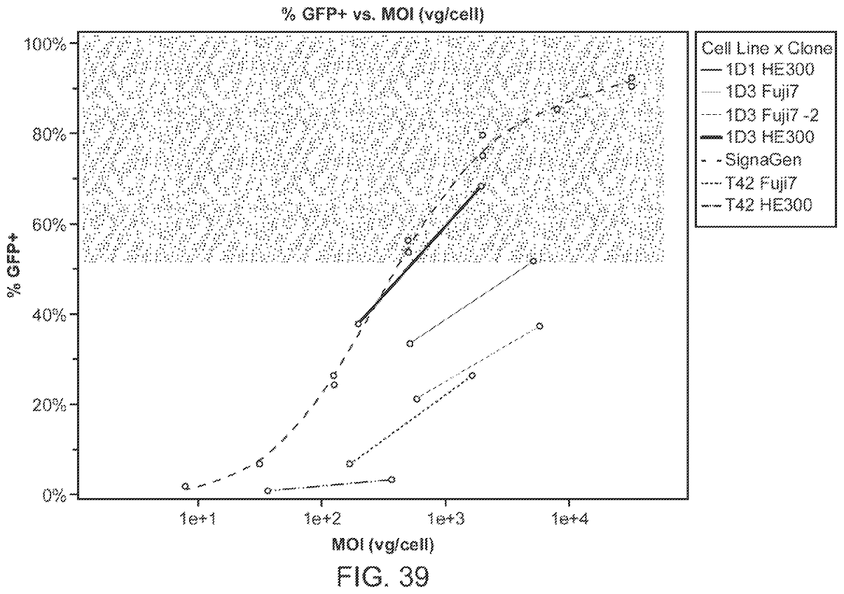

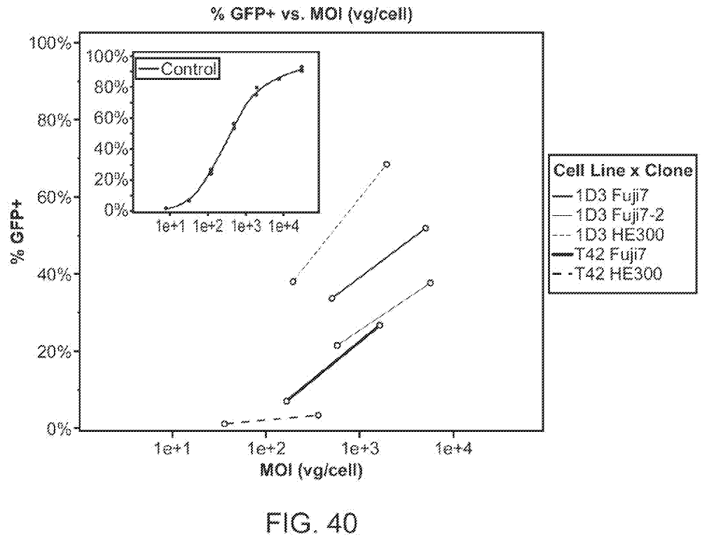

In some embodiments, the inducible helper polynucleotide construct is as shown at left or at right in FIG. 25. Multiple inducible helper polynucleotide constructs are contemplated herein. In some embodiments, the elements of the inducible helper polynucleotide constructs can be in one or more separate constructs. In some embodiments, said inducible helper polynucleotide constructs encode for one or more adenoviral helper proteins, such as VA RNA, E2A, E4, or any combination thereof. In some embodiments, the present disclosure provides for an inducible polynucleotide construct encoding for a mutated VA RNA gene sequence. In some embodiments, the mutations to VA RNA render its internal promoters inactive. For example, as shown in FIG. 25 (at left), the inducible helper polynucleotide construct may comprise from 5′ to 3′ a first part of a U6 promoter sequence, a first lox sequence, a stuffer sequence, a second lox sequence, and a second part of a U6 promoter sequence. The stuffer sequence may be any polynucleotide sequence and is excised by Cre. Cre may be exogenously provided, such as in the form of Cre gesicles. Cre may also be encoded for in the same inducible helper polynucleotide construct and expression of Cre may be conditioned on the presence of at least two triggering agents, such as doxycycline and tamoxifen. Cre may be a hormone activated Cre.

In other embodiments, instead of a mutated VA RNA gene sequence, the inducible helper constructs may comprise a constitutively expressed VA RNA that is not mutated, for example, as shown in FIG. 25 (at right).

In some embodiments, the inducible helper polynucleotide construct also encodes for one or more helper proteins, a self-excising element upstream of the one or more helper proteins, and an inducible promoter upstream of the self-excising element. Expression of the self-excising element may be driven by a Tet-On-3G system. For example, the construct may comprise a Tet-On 3G gene sequence, wherein expression is driven by an EF1alpha promoter. The EF1alpha promoter may be a mutated EF1alpha promoter. The mutated EF1alpha promoter can have a sequence of:

(SEQ ID NO: 20)
ggatctgcgatcgctccggtgcccgtcagtgggcagagcgcacatcgcc
cacagtccccgagaagttggggggaggggtcggcaattgaacgggtgcc
tagagaaggtggcgcggggtaaactgggaaagtgatgtcgtgtactggc
tccgcctttttcccgaggggggggagaaccgtatgtaagtgcagtagtc
gccgtgaacgttctttttcgcaacgggtttgccgccagaacacagctga
agcttcgaggggctcgcatctctccttcacgcgcccgccgccctacctg
aggccgccatccacgccggttgagtcgcgttctgccgcctcccgcctgt
ggtgcctcctgaactgcgtccgccgtctaggtaagtttaaagctcaggt
cgagaccgggcctttgtccggcgctcccttggagcctacctagactcag
ccggctctccacgctttgcctgaccctgcttgctcaactctacgtcttt
gtttcgttttctgttctgcgccgttacagatccaagctgtgaccggcgc
ctac.

In the presence of a first triggering agent, such as doxycycline, Tet-On-3G is able to bind the Tet inducible promoter. Upon this binding event, the Tet inducible promoter drives expression of the self-excising element. In some embodiments, the self-excising element is a hormone activated Cre. In the presence of a second triggering agent, such as tamoxifen, and upon expression of Cre, Cre self-excises itself leading to expression of downstream adenoviral helper proteins. Thus, mammalian cell lines stably integrated with the inducible helper constructs disclosed herein only express adenoviral helper proteins in the presence of at least two triggering agents (e.g., doxycycline and tamoxifen).

In some embodiments, an inducible helper construct is a polynucleotide construct coding for: a) one or more helper proteins; and b) a self-excising element upstream of the one or more helper proteins. In some embodiments, the self-excising element is operably linked to a constitutive promoter. In some embodiments, the constitutive promoter is EF1 alpha promoter or human cytomegalovirus promoter. In some embodiments, a sequence coding for the self-excising element comprises a poly A sequence. In some embodiments, the self-excising element is a recombinase. In some embodiments, the recombinase is a site-specific recombinase. In some embodiments, the recombinase is fused to a ligand binding domain. In some embodiments, the recombinase is Cre polypeptide or flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the self-excising element translocates to the nucleus in the presence of a triggering agent. In some embodiments, the triggering agent is an estrogen receptor ligand. In some embodiments, the triggering agent is a selective estrogen receptor modulator (SERM). In some embodiments, the triggering agent is tamoxifen. In some embodiments, the recombinase is flanked by recombination sites. In some embodiments, the recombination sites are lox sites or flippase recognition target (FRT) sites. In some embodiments, the lox sites are loxP sites. In some embodiments, the self-excising element is excised upon administration of the triggering agent, thereby operably linking the constitutive promoter to the one or more helper proteins. In some embodiments, the inducible helper construct lacks sequences coding for a tetracycline inducible system (e.g., a tetracycline-responsive promoter element (TRE) and/or a reverse tetracycline-controlled transactivator (rTA)). In some embodiments, the inducible helper construct lacks sequences coding for a tetracycline-inducible system, an ecdysone-inducible system, or a cumate-inducible system.

In some embodiments, an inducible helper construct is a polynucleotide construct coding for: a) one or more helper proteins; b) a self-excising element upstream of the one or more helper proteins; and c) an inducible promoter upstream of the self-excising element. In some embodiments, the self-excising element is operably linked to the inducible promoter. In some embodiments, expression of the self-excising element is driven by the inducible promoter.

In some embodiments, an inducible helper construct is a polynucleotide construct coding for: a) one or more helper proteins; b) a recombinase; and c) an inducible promoter upstream of the one or more helper proteins; and d) an inducible promoter upstream the recombinase. In some embodiments, the recombinase is operably linked to the inducible promoter. In some embodiments, the one or more helper proteins are operably linked to the inducible promoter. In some embodiments, expression of the recombinase is driven by the inducible promoter. In some embodiments, expression of the one or more helper proteins is driven by the inducible promoter.

In some embodiments, the inducible promoter is a tetracycline-responsive promoter element (TRE). In some embodiments, the TRE comprises Tet operator (tetO) sequence concatemers fused to a minimal promoter. In some embodiments, the minimal promoter is a human cytomegalovirus promoter. In some embodiments, the minimal promoter comprises a sequence having at least 70%, 80%, 90%, 95%, 99%, or 100% sequence identity to any one of SEQ ID NOs: 63-68. In some embodiments, the inducible promoter comprises a sequence having at least 70%, 80%, 90%, 95%, 99%, or 100% sequence identity to any one of SEQ ID NOs: 22, 46-48, or 50-62. In some embodiments, transcription is activated from the inducible promoter upon binding of an activator. In some embodiments, the activator binds to the inducible promoter in the presence of a first triggering agent. In some embodiments, further comprising an activator. In some embodiments, the activator is operably linked to a constitutive promoter. In some embodiments, the constitutive promoter is EF1 alpha promoter or human cytomegalovirus promoter. In some embodiments, the EF1alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the activator is reverse tetracycline-controlled transactivator (rTA) comprising a Tet Repressor binding protein (TetR) fused to a VP16 transactivation domain. In some embodiments, the rTA comprises four mutations in the tetR DNA binding moiety. In some embodiments, the rTA comprises a sequence having at least 70%, 80%, 90%, 95%, 99%, or 100% sequence identity to any one of SEQ ID NOs: 21, 40-45, or 69-86, or variants thereof.

In some embodiments, the inducible promoter is bound by a repressor in the absence of a first triggering agent. In some embodiments, the inducible promoter is activated in the presence of a first triggering agent. In some embodiments, the first triggering agent binds to the repressor. In some embodiments, the repressor is a tetracycline-controlled transactivator. In some embodiments, further comprising the repressor. In some embodiments, the repressor is operably linked to a constitutive promoter. In some embodiments, further comprising a tetracycline-controlled transactivator. In some embodiments, the tetracycline-controlled transactivator is operably linked to a constitutive promoter. In some embodiments, the constitutive promoter is EF1 alpha promoter. In some embodiments, the EF1alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the tetracycline-controlled transactivator is unbound in the presence of a first triggering agent. In some embodiments, the tetracycline-controlled transactivator does not bind to the inducible promoter in the presence of a first triggering agent. In some embodiments, the constitutive promoter is EF1alpha promoter. In some embodiments, the EF1alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, transcription is activated from the inducible promoter upon binding of the first triggering agent to the repressor. In some embodiments, the repressor binds to the first triggering agent. In some embodiments, the first triggering agent is a tetracycline. In some embodiments, the tetracycline is doxycycline.

In some embodiments, wherein the inducible promoter is a cumate operator sequence. In some embodiments, the cumate operator sequence is downstream of a constitutive promoter. In some embodiments, the constitutive promoter is a human cytomegalovirus promoter. In some embodiments, wherein the inducible promoter is bound by a cymR repressor in the absence of a first triggering agent. In some embodiments, the inducible promoter is activated in the presence of a first triggering agent. In some embodiments, the first triggering agent binds to the cymR repressor. In some embodiments, the cumate inducible system further comprises a cymR repressor. In some embodiments, the cymR repressor is operably linked to a constitutive promoter. In some embodiments, the constitutive promoter is EF1alpha promoter. In some embodiments, the EF1alpha promoter comprises at least one mutation. In some embodiments, the constitutive promoter comprises a sequence having at least 70%, 80%, 90%, 95%, 99%, or 100% sequence identity with SEQ ID NO: 20. In some embodiments, the first triggering agent is a cumate.

In some embodiments, a sequence coding for the self-excising element comprises a poly A sequence. In some embodiments, the self-excising element is a recombinase. In some embodiments, the recombinase is a site-specific recombinase. In some embodiments, the recombinase is fused to a ligand binding domain. In some embodiments, the recombinase is Cre polypeptide or flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the self-excising element translocates to the nucleus in the presence of a second triggering agent. In some embodiments, the second triggering agent is an estrogen receptor ligand. In some embodiments, the second triggering agent is a selective estrogen receptor modulator (SERM). In some embodiments, the second triggering agent is tamoxifen. In some embodiments, the recombinase is flanked by recombination sites. In some embodiments, the recombination sites are lox sites or flippase recognition target (FRT) sites. In some embodiments, the lox sites are loxP sites.

In some embodiments, the one or more adenoviral helper proteins comprise E2A and E4. In some embodiments, the one or more adenoviral helper proteins further comprises a protein tag. In some embodiments, the protein tag is a FLAG-tag. In some embodiments, the E2A is FLAG-tagged E2A. In some embodiments, the sequence coding for E2 and the sequence coding for E4 are separated by an internal ribosome entry site (IRES) or by P2A.

In some embodiments, the inducible helper construct further comprises a sequence coding for a selectable marker.

In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the helper construct further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the helper construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, an inducible helper construct further comprises a sequence coding for VA RNA. In some embodiments, the VA RNA is on a separate construct from the sequences encoding one or more helper proteins. In some embodiments, the VA RNA is operably linked to a constitutive promoter or an inducible promoter. In some embodiments, the constitutive promoter is EF1alpha promoter or human cytomegalovirus promoter. In some embodiments, the inducible promoter is a tetracycline-inducible promoter, an ecdysone-inducible promoter, or a cumate-inducible promoter. In some embodiments, the sequence coding for VA RNA is a transcriptionally dead sequence. In some embodiments, the sequence coding for VA RNA comprises at least two mutations in the internal promoter. In some embodiments, the expression of the VA RNA is under the control of an RNA polymerase III promoter. In some embodiments, the expression of the VA RNA is under the control of an interrupted RNA polymerase III promoter. In some embodiments, the expression of the VA RNA is under the control of a U6 or U7 promoter. In some embodiments, the expression of the VA RNA is under the control of an interrupted U6 or U7 promoter. In some embodiments, the polynucleotide construct comprises upstream of the sequence coding for VA RNA gene sequence, from 5′ to 3′: a) a first part of a U6 or U7 promoter sequence; b) a first recombination site; c) a stuffer sequence; d) a second recombination site; e) a second part of a U6 or U7 promoter sequence. In some embodiments, the stuffer sequence is excisable by the recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter.

In some embodiments, the gene encodes a detectable marker or a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the stuffer sequence further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the stuffer sequence further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, the detectable marker comprises a luminescent marker or a fluorescent marker. In some embodiments, the fluorescent marker is GFP, EGFP, RFP, CFP, BFP, YFP, or mCherry. In some embodiments, the first recombination site is a first lox sequence and the second recombination site is a second lox sequence. In some embodiments, the first lox sequence is a first loxP site and the second lox sequence is a second loxP site. In some embodiments, the first recombination site is a first FRT site and the second recombination site is a second FRT site.

In some embodiments, an inducible helper construct is in a vector. In some embodiments, an inducible helper construct is in a plasmid. In some embodiments, an inducible helper construct is in a bacterial artificial chromosome or yeast artificial chromosome. In some embodiments, an inducible helper construct is a synthetic nucleic acid construct. In some embodiments, an inducible helper construct comprises a sequence having at least 70%, 80%, 90%, 95%, 99%, or 100% sequence identity to any one of SEQ ID NO: 9-SEQ ID NO: 19, SEQ ID 23-SEQ ID NO: 32, SEQ ID NO: 35, SEQ ID NO: 90-SEQ ID NO: 99, SEQ ID NO: 101-SEQ ID NO: 109, SEQ ID NO: 112-SEQ ID NO: 131, SEQ ID NO: 137, or SEQ ID NO: 138, or any combination thereof. In some embodiments, an inducible helper construct has at least 70%, 80%, 90%, 95%, 99%, or 100% sequence identity to any one of SEQ ID NO: 9-SEQ ID NO: 19, SEQ ID 23-SEQ ID NO: 32, SEQ ID NO: 35, SEQ ID NO: 90-SEQ ID NO: 99, SEQ ID NO: 101-SEQ ID NO: 109, SEQ ID NO: 112-SEQ ID NO: 131, SEQ ID NO: 137, or SEQ ID NO: 138, or any combination thereof.

In some embodiments, an inducible helper construct comprises a polynucleotide construct coding for a VA RNA. In some embodiments, the VA RNA is on a separate construct from the sequences encoding one or more helper proteins. In some embodiments, the VA RNA is operably linked to a constitutive promoter or an inducible promoter. In some embodiments, the constitutive promoter is EF1alpha promoter or human cytomegalovirus promoter. In some embodiments, the inducible promoter is a tetracycline-inducible promoter, an ecdysone-inducible promoter, or a cumate-inducible promoter. In some embodiments, an inducible helper construct comprises a polynucleotide construct coding for a VA RNA, wherein a sequence coding for the VA RNA comprises at least two mutations in an internal promoter. In some embodiments, a separate polynucleotide construct codes for a VA RNA, wherein a sequence coding for the VA RNA comprises at least two mutations in an internal promoter. In some embodiments, the sequence coding for the VA RNA comprises a sequence coding for a transcriptionally dead VA RNA. In some embodiments, the sequence coding for the VA RNA comprises a deletion of from about 5-10 nucleotides in the promoter region. In some embodiments, the sequence coding for the VA RNA comprises at least one mutation. In some embodiments, the at least one mutation is in the A Box promoter region. In some embodiments, the at least one mutation is in the B Box promoter region. In some embodiments, the at least one mutation is G16A and G60A. In some embodiments, the expression of the VA RNA is under the control of an RNA polymerase III promoter. In some embodiments, the expression of the VA RNA is under the control of an interrupted RNA polymerase III promoter. In some embodiments, the expression of the VA RNA is under the control of a U6 or U7 promoter. In some embodiments, the expression of the VA RNA is under the control of an interrupted U6 or U7 promoter. In some embodiments, the polynucleotide construct comprises upstream of the VA RNA gene sequence, from 5′ to 3′: a) a first part of a U6 or U7 promoter sequence; b) a first recombination site; c) a stuffer sequence; d) a second recombination site; e) a second part of a U6 or U7 promoter sequence. In some embodiments, the stuffer sequence is excisable by a recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter. In some embodiments, the gene encodes a detectable marker or a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the stuffer sequence further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the stuffer sequence further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, the detectable marker comprises a luminescent marker or a fluorescent marker. In some embodiments, the fluorescent marker is GFP, EGFP, RFP, CFP, BFP, YFP, or mCherry. In some embodiments, an inducible helper construct comprises a polynucleotide construct coding for a VA RNA or the VA RNA construct further comprising a sequence coding for a recombinase. In some embodiments, the recombinase is exogenously provided. In some embodiments, the recombinase is a site-specific recombinase. In some embodiments, the recombinase is a Cre polypeptide or a Flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the first recombination site is a first lox sequence and the second recombination site is a second lox sequence. In some embodiments, the first lox sequence is a first loxP site and the second lox sequence is a second loxP site. In some embodiments, the first recombination site is a first FRT site and the second recombination site is a second FRT site. In some embodiments, the construct comprising the VA RNA as described herein further comprises a sequence coding for a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the construct comprising the VA RNA further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the construct comprising the VA RNA further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, an inducible helper construct comprises a polynucleotide construct coding for a VA RNA or the VA RNA construct is in a vector. In some embodiments, an inducible helper construct comprises a polynucleotide construct coding for a VA RNA or the VA RNA construct is in a plasmid. In some embodiments, an inducible helper construct comprises a polynucleotide construct coding for a VA RNA or the VA RNA construct is in a bacterial artificial chromosome or yeast artificial chromosome. In some embodiments, an inducible helper construct comprises a polynucleotide construct coding for a VA RNA or the VA RNA construct is a synthetic nucleic acid construct. In some embodiments, an inducible helper construct comprises a sequence having at least 70%, 80%, 90%, 95%, 99%, or 100% sequence identity to any one of SEQ ID NO: 13-SEQ ID NO: 19 or SEQ ID 23-SEQ ID NO: 26. In some embodiments, an inducible helper construct has at least 70%, 80%, 90%, 95%, 99%, or 100% sequence identity to any one of SEQ ID NO: 13-SEQ ID NO: 19 or SEQ ID 23-SEQ ID NO: 26. In some embodiments, a VA RNA construct comprises a sequence having at least 70%, 80%, 90%, 95%, 99%, or 100% sequence identity to any one of SEQ ID NO: 13-SEQ ID NO: 19 or SEQ ID 23-SEQ ID NO: 26. In some embodiments, a VA RNA construct has a sequence having at least 70%, 80%, 90%, 95%, 99%, or 100% sequence identity to any one of SEQ ID NO: 13-SEQ ID NO: 19 or SEQ ID 23-SEQ ID NO: 26.

### Polynucleotide Encoding a Payload

In some embodiments, the third integrated synthetic construct (also referred to as Construct 3) comprises the coding sequence for an expressible payload and a third mammalian cell selection element. In the exemplary embodiments shown in FIG. 4, the expressible payload is under the control of a constitutive promoter. This construct can be referred to as construct 3 or payload construct, interchangeably.

In some embodiments, the expressible payload encodes a guide RNA. In certain embodiments, the guide RNA directs RNA editing. In some embodiments, the guide RNA directs Cas-mediated DNA editing. In some embodiments, the guide RNA directs ADAR-mediated RNA editing. In some embodiments, the third integrated synthetic construct comprises a sequence encoding for any of the expressible payloads disclosed herein. For example, said sequence can encode for any therapeutic. For example, the therapeutic may be a transgene, a guide RNA, an antisense RNA, an oligonucleotide, an mRNA, a miRNA, a shRNA, a tRNA suppressor, a CRISPR-Cas protein, any gene editing enzyme, or any combination thereof. In some embodiments, the transgene encodes for progranulin. In some embodiments, the tRNA suppressor is capable of suppressing an opal stop codon. In some embodiments, the tRNA suppressor is capable of suppressing an ochre stop codon. In some embodiments, the tRNA suppressor is capable of suppressing an amber stop codon. In some embodiments, the third integrated synthetic construct comprises sequences encoding for more than one of the expressible payloads disclosed herein. For example, the third integrated synthetic construct comprise 2 gRNA, 3 gRNA, 4 gRNA, 5 gRNA, 6 gRNA, 7 gRNA, 8 gRNA, 9 gRNA, or 10 gRNA. These gRNAs can all be the same, all be different, or any combination of the same and different. For example, the third integrated synthetic construct comprise 2 suppressor tRNAs, 3 suppressor tRNAs, 4 suppressor tRNAs, 5 suppressor tRNAs, 6 suppressor tRNAs, 7 suppressor tRNAs, 8 suppressor tRNAs, 9 suppressor tRNAs, or 10 suppressor tRNAs. These suppressor tRNAs can all be the same, all be different, or any combination of the same and different.

In some embodiments, the expressible payload encodes a protein. In certain embodiments, the expressible payload is an enzyme, useful for replacement gene therapy. In some embodiments, the protein is a therapeutic antibody. In some embodiments, the protein is a vaccine immunogen. In particular embodiments, the vaccine immunogen is a viral protein.

In some embodiments, the expressible payload is a homology construct for homologous recombination.

In various embodiments, the third mammalian cell selection element is an auxotrophic selection element.

In some embodiments, the payload construct comprises a sequence having at least 70%, 80%, 90%, 95%, 99%, or 100% sequence identity to SEQ ID NO: 33 or SEQ ID NO: 139. In some embodiments, the payload construct has at least 70%, 80%, 90%, 95%, 99%, or 100% sequence identity to SEQ ID NO: 147. In some embodiments, the payload construct has at least 70%, 80%, 90%, 95%, 99%, or 100% sequence identity to SEQ ID NO: 149. In some embodiments, the payload construct has at least 70%, 80%, 90%, 95%, 99%, or 100% sequence identity to SEQ ID NO: 151. In some embodiments, the payload construct has at least 70%, 80%, 90%, 95%, 99%, or 100% sequence identity to SEQ ID NO: 153. In some embodiments, a plasmid comprises the payload construct and comprises a sequence having at least 70%, 80%, 90%, 95%, 99%, or 100% sequence identity to SEQ ID NO: 33. In some embodiments, a plasmid comprises the payload construct and comprises a sequence having at least 70%, 80%, 90%, 95%, 99%, or 100% sequence identity to SEQ ID NO: 147. In some embodiments, a plasmid comprises the payload construct and comprises a sequence having at least 70%, 80%, 90%, 95%, 99%, or 100% sequence identity to SEQ ID NO: 149. In some embodiments, a plasmid comprises the payload construct and comprises a sequence having at least 70%, 80%, 90%, 95%, 99%, or 100% sequence identity to SEQ ID NO: 151. In some embodiments, a plasmid comprises the payload construct and comprises a sequence having at least 70%, 80%, 90%, 95%, 99%, or 100% sequence identity to SEQ ID NO: 153. In some embodiments, the payload construct comprises a sequence having at least 70%, 80%, 90%, 95%, 99%, or 100% sequence identity to SEQ ID NO: 33 or SEQ ID NO: 139, wherein SEQ ID NO: 34 in SEQ ID NO: 33 or SEQ ID NO: 139 is replaced with a sequence of the payload of interest. In some embodiments, the payload construct comprises a sequence having at least 70%, 80%, 90%, 95%, 99%, or 100% sequence identity to SEQ ID NO: 146. In some embodiments, the payload construct comprises a sequence having at least 70%, 80%, 90%, 95%, 99%, or 100% sequence identity to SEQ ID NO: 148. In some embodiments, the payload construct comprises a sequence having at least 70%, 80%, 90%, 95%, 99%, or 100% sequence identity to SEQ ID NO: 150. In some embodiments, the payload construct comprises a sequence having at least 70%, 80%, 90%, 95%, 99%, or 100% sequence identity to SEQ ID NO: 152.

In some embodiments, the payload construct comprises a sequence of a payload flanked by ITR sequences. In some embodiments, expression of the sequence of the payload is driven by a constitutive promoter or an inducible promoter. In some embodiments, the promoter and sequence of the payload are flanked by ITR sequences. In some embodiments, the payload construct comprises a sequence having at least 70%, 80%, 90%, 95%, 99%, or 100% sequence identity to SEQ ID NO: 146. In some embodiments, the payload construct comprises a sequence having at least 70%, 80%, 90%, 95%, 99%, or 100% sequence identity to SEQ ID NO: 148. In some embodiments, the payload construct comprises a sequence having at least 70%, 80%, 90%, 95%, 99%, or 100% sequence identity to SEQ ID NO: 150. In some embodiments, the payload construct comprises a sequence having at least 70%, 80%, 90%, 95%, 99%, or 100% sequence identity to SEQ ID NO: 152.

In some embodiments, the sequence of the payload comprises a polynucleotide sequence coding for a gene. In some embodiments, the gene codes for a selectable marker or detectable marker. In some embodiments, the gene codes for a therapeutic polypeptide or transgene. In some embodiments, the therapeutic polypeptide or transgene is progranulin. In some embodiments, the sequence of the payload comprises a polynucleotide sequence coding for a therapeutic polynucleotide. In some embodiments, the therapeutic polynucleotide is a tRNA suppressor or a guide RNA. In some embodiments, the tRNA suppressor is capable of suppressing an opal stop codon. In some embodiments, the tRNA suppressor is capable of suppressing an ochre stop codon. In some embodiments, the tRNA suppressor is capable of suppressing an amber stop codon. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the guide RNA, when bound to a target RNA, recruits an ADAR protein for editing of the target RNA. In some embodiments, the Cas protein is catalytically inactive Cas protein. In some embodiments, the payload construct is stably integrated into the genome of the cell. In some embodiments, a plurality of the payload construct are stably integrated into the genome of the cell. In some embodiments, the plurality of the payload constructs are separately stably integrated into the genome of the cell.

In some embodiments, the payload construct further comprises a sequence coding for a selectable marker or detectable marker outside of the ITR sequences. In some embodiments, expression of the selectable marker or detectable marker outside of the ITR sequences is driven by a promoter. The promoter can be a constitutive promoter or an inducible promoter. In some embodiments, the constitutive promoter is EF1alpha promoter or human cytomegalovirus promoter. In some embodiments, the inducible promoter is a tetracycline-inducible promoter, an ecdysone-inducible promoter, or a cumate-inducible promoter. In some embodiments, the selectable marker is a mammalian cell selection element (e.g., a third mammalian cell selection element). In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the payload construct further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the payload construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, the selectable marker is outside of the ITR sequences on the payload construct. In some embodiments, the selectable marker outside of the ITR sequences is a split intein linked to an N-terminus of the auxotrophic protein or split intein linked to a C-terminus of the auxotrophic protein. In some embodiments, the selectable marker outside of the ITR sequences is a leucine zipper linked to an N-terminus of the auxotrophic or leucine zipper linked to a C-terminus of the auxotrophic. In some embodiments, the selectable marker outside of the ITR sequences is a split intein linked to an N-terminus of the antibiotic resistance protein or split intein linked to a C-terminus of the antibiotic resistance protein. In some embodiments, the selectable marker outside of the ITR sequences is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the payload construct further comprises a spacer between the 5′ ITR and the promoter/selectable marker or promoter/detectable marker outside of the ITR sequences. In some embodiments, the payload construct further comprises a spacer between the 3′ ITR and the promoter/selectable marker or promoter/detectable marker outside of the ITR sequences. In some embodiments, the spacer ranges in length from 500 base pairs to 5000 base pairs, including any length within this range such as 500, 525, 550, 575, 600, 625, 650, 675, 700, 725, 750, 775, 800, 825, 850, 875, 900, 925, 950, 975, 1000, 1250, 1500, 1750, 2000, 2225, 2500, 2750, 3000, 3250, 3500, 3750, 4000, 4250, 4500, 4750, or 5000 base pairs. In some embodiments, the spacer length is a sufficient length for decreasing reverse packaging of the selectable marker or detectable marker that is outside the ITR sequences.

In some embodiments, the third integrated synthetic construct comprising the coding sequence for a payload and a selectable marker or detectable marker is further engineered to remove locations having the potential for Rep-mediated nicking. For example, a location having the potential for Rep-mediated nicking is a location having the sequence CAGTGAGCGAGCGAGCGCGCAG (SEQ ID NO: 87); a sequence comprising GAGC repeats; or the sequence GATGGAGTTGGCCACTCCCTC (SEQ ID NO: 89). These sequences can be engineered to prevent binding of Rep proteins for Rep-mediated nicking. In some embodiments, the location having the potential for Rep-mediated nicking that is engineered to prevent binding of Rep proteins for Rep-mediated nicking is in a region within 100 nucleotides of an ITR sequence. In some embodiments, the location having the potential for Rep-mediated nicking that is engineered to prevent binding of Rep proteins for Rep-mediated nicking is in a region within 200 nucleotides of an ITR sequence. In some embodiments, the location having the potential for Rep-mediated nicking that is engineered to prevent binding of Rep proteins for Rep-mediated nicking is in a region within 300 nucleotides of an ITR sequence. In some embodiments, the location having the potential for Rep-mediated nicking that is engineered to prevent binding of Rep proteins for Rep-mediated nicking is in a region within 400 nucleotides of an ITR sequence. In some embodiments, the location having the potential for Rep-mediated nicking that is engineered to prevent binding of Rep proteins for Rep-mediated nicking is in a region within 500 nucleotides of an ITR sequence. In some embodiments, the location having the potential for Rep-mediated nicking that is engineered to prevent binding of Rep proteins for Rep-mediated nicking is in a region within 600, 625, 650, 675, 700, 725, 750, 775, 800, 825, 850, 875, 900, 925, 950, 975, 1000, 1250, 1500, 1750, 2000, 2225, 2500, 2750, 3000, 3250, 3500, 3750, 4000, 4250, 4500, 4750, or 5000 nucleotides of an ITR sequence.

In some embodiments, a payload construct comprises a polynucleotide construct coding for a VA RNA. In some embodiments, the VA RNA is operably linked to a constitutive promoter or an inducible promoter. In some embodiments, the constitutive promoter is EF1 alpha promoter or human cytomegalovirus promoter. In some embodiments, the inducible promoter is a tetracycline-inducible promoter, an ecdysone-inducible promoter, or a cumate-inducible promoter. In some embodiments, a payload construct comprises a polynucleotide construct coding for a VA RNA, wherein a sequence coding for the VA RNA comprises at least two mutations in an internal promoter. In some embodiments, a separate polynucleotide construct codes for a VA RNA, wherein a sequence coding for the VA RNA comprises at least two mutations in an internal promoter. In some embodiments, the sequence coding for the VA RNA comprises a sequence coding for a transcriptionally dead VA RNA. In some embodiments, the sequence coding for the VA RNA comprises a deletion of from about 5-10 nucleotides in the promoter region. In some embodiments, the sequence coding for the VA RNA comprises at least one mutation. In some embodiments, the at least one mutation is in the A Box promoter region. In some embodiments, the at least one mutation is in the B Box promoter region. In some embodiments, the at least one mutation is G16A and G60A. In some embodiments, the expression of the VA RNA is under the control of an RNA polymerase III promoter. In some embodiments, the expression of the VA RNA is under the control of an interrupted RNA polymerase III promoter. In some embodiments, the expression of the VA RNA is under the control of a U6 or U7 promoter. In some embodiments, the expression of the VA RNA is under the control of an interrupted U6 or U7 promoter. In some embodiments, the polynucleotide construct comprises upstream of the VA RNA gene sequence, from 5′ to 3′: a) a first part of a U6 or U7 promoter sequence; b) a first recombination site; c) a stuffer sequence; d) a second recombination site; e) a second part of a U6 or U7 promoter sequence. In some embodiments, the stuffer sequence is excisable by a recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter. In some embodiments, the gene encodes a detectable marker or a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the stuffer sequence further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the stuffer sequence further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, the detectable marker comprises a luminescent marker or a fluorescent marker. In some embodiments, the fluorescent marker is GFP, EGFP, RFP, CFP, BFP, YFP, or mCherry. In some embodiments, an inducible helper construct comprises a polynucleotide construct coding for a VA RNA or the VA RNA construct further comprising a sequence coding for a recombinase. In some embodiments, the recombinase is exogenously provided. In some embodiments, the recombinase is a site-specific recombinase. In some embodiments, the recombinase is a Cre polypeptide or a Flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the first recombination site is a first lox sequence and the second recombination site is a second lox sequence. In some embodiments, the first lox sequence is a first loxP site and the second lox sequence is a second loxP site. In some embodiments, the first recombination site is a first FRT site and the second recombination site is a second FRT site. In some embodiments, the construct comprising the VA RNA as described herein further comprises a sequence coding for a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the construct comprising VA RNA further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the construct comprising the VA RNA further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, an inducible helper construct comprises a polynucleotide construct coding for a VA RNA or the VA RNA construct is in a vector. In some embodiments, an inducible helper construct comprises a polynucleotide construct coding for a VA RNA or the VA RNA construct is in a plasmid. In some embodiments, an inducible helper construct comprises a polynucleotide construct coding for a VA RNA or the VA RNA construct is in a bacterial artificial chromosome or yeast artificial chromosome. In some embodiments, an inducible helper construct comprises a polynucleotide construct coding for a VA RNA or the VA RNA construct is a synthetic nucleic acid construct. In some embodiments, an inducible helper construct comprises a sequence having at least 70%, 80%, 90%, 95%, 99%, or 100% sequence identity to any one of SEQ ID NO: 13-SEQ ID NO: 19 or SEQ ID 23-SEQ ID NO: 26. In some embodiments, an inducible helper construct has at least 70%, 80%, 90%, 95%, 99%, or 100% sequence identity to any one of SEQ ID NO: 13-SEQ ID NO: 19 or SEQ ID 23-SEQ ID NO: 26. In some embodiments, a VA RNA construct comprises a sequence having at least 70%, 80%, 90%, 95%, 99%, or 100% sequence identity to any one of SEQ ID NO: 13-SEQ ID NO: 19 or SEQ ID 23-SEQ ID NO: 26. In some embodiments, a VA RNA construct has a sequence having at least 70%, 80%, 90%, 95%, 99%, or 100% sequence identity to any one of SEQ ID NO: 13-SEQ ID NO: 19 or SEQ ID 23-SEQ ID NO: 26.

### Host Production Cell

The present disclosure further provides host cells comprising the vector system described herein. A subject host cell can be an isolated cell, e.g., a cell in in vitro culture. A subject host cell is useful for producing rAAV virions, as described below. A subject host cell useful for producing rAAV virions can be any cell that is capable of expressing proteins from a p5 promoter. Where a subject host cell is used to produce rAAV virions, it is referred to as a “packaging cell.” In some cases, a subject host cell is stably genetically modified with the vector system. In other cases, a subject host cell is transiently genetically modified with the vector system.

The vector system described herein can be used in a variety of host cells for rAAV virion production. For example, suitable host cells that have been transfected with the vector system are rendered capable of producing rAAV virions. The first and second, and third polynucleotide constructs of the vector system can be introduced into a host cell, either simultaneously or serially, using established transfection techniques, including, but not limited to, electroporation, calcium phosphate precipitation, liposome-mediated transfection, and the like. In some embodiments, the first and second polynucleotide constructs of the vector system are introduced into a host cell, and the third polynucleotide construct comprising the expressible payload is introduced later when production of the payload is desired.

A subject host cell is generated by introducing the vector system into any of a variety of cells, e.g., mammalian cells, including, without limitation, murine cells, and primate cells (e.g., human cells). Suitable mammalian cells include, but are not limited to, primary cells and cell lines, where suitable cell lines include, but are not limited to, 293 cells, COS cells, HeLa cells, Vero cells, 3T3 mouse fibroblasts, C3H10T1/2 fibroblasts, CHO cells, and the like. Non-limiting examples of suitable host cells include, e.g., HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like. A subject host cell can also be made using a baculovirus to infect insect cells such as Sf9 cells, which produce AAV (see, e.g., U.S. Pat. Nos. 7,271,002 and 8,945,918). In some embodiments, a host cell is any cell capable of activating a p5 of sequence encoding a Rep protein.

In typical embodiments, the production host cell is a mammalian cell line that expresses adenovirus E1A and E1B. In particular embodiments, the cell is a human embryonic kidney (HEK) 293 cell line or derivatives thereof (HEK293T cells, HEK293F cells), a human HeLa cell line that expresses E1A and E1B, a Chinese hamster ovary (CHO) cell line that expresses E1A and E1B, or a Vero cell that expresses adenovirus E1A and E1B. In particular embodiments, the host cell is a HEK293 cell line.

In certain embodiments, the host cell is or is genetically altered to be deficient in an enzyme required for production of a molecule required for cell growth, for example, an enzyme required for catalyzing production of a cofactor or nutrient. In certain embodiments, the host cell is DHFR null. In specific embodiments, the host cell is a DHFR null HEK293 cell. In some embodiments, the host cell is GS null. In some embodiments, the host cell is a GS null HEK293.

In some embodiments, the host cell expresses or is genetically modified to express GTP-CH1.

In some embodiments, the HEK293 cell expresses AAV E1A and E1B. In the presence of doxycycline and tamoxifen, the ER2 Cre is excised from the first integrated synthetic construct, thereby permitting expression of AAV E2A and E4. The self-excised ER2 Cre recombines by virtue of the lox sites flanking the EGFP cassette in the second integrated synthetic construct, thereby removing the EGFP segment from the second spacer element in the integrated second synthetic construct. As such, any cells comprising only the second integrated synthetic construct will be EGFP signal positive whereas cells comprising both the first and second integrated synthetic constructs will be EGFP signal negative, following the addition of the triggering agents. Absence of EGFP signal indicates successful transfection of both the first and second integrated synthetic constructs in a cell. This is further ensured by antibiotic resistance selection, e.g., blasticidin resistance.

Additionally, removal of the EGFP cassette provides for the functional expression of Rep and Cap proteins, which can be linked to a first selectable marker, e.g., a first DHFR selection element, e.g., Z-Cter DHFR. The first selectable marker is capable of associating with a second selectable marker, e.g., a second DHFR selection element, e.g., a Z-Cter DHFR is capable of associating with a second DHFR selection element, e.g., Z-Nter DHFR, present in the third integrated synthetic construct to form an active molecule that allows the cell to survive in a selection medium, e.g., HT lacking media selection. The first selectable marker and second selectable marker can be any selectable marker as described herein, wherein expression of the first selectable marker and expression of the second selectable marker form an active molecule (e.g., a functional enzyme) that allows the cell to survive in a selection medium (e.g., a selection media deficient in the product produced by the functional enzyme).

In some embodiments, the third integrated synthetic construct comprises a payload. The payload can be a guide RNA (FIGS. 4 and 5B), an HDR homology region, or a gene of interest.

In some embodiments, one or more of the synthetic nucleic acid constructs are integrated into the genome of a production host cell. In some embodiments, the integration of a construct into a chromosome is site-specific. Any method known in the art for directing integration into the genome may be used. For example, a polynucleotide construct can be cloned into a lentivirus vector that integrates into the nuclear genome of the cell. Alternatively, a transposon system, a clustered regularly interspersed short palindromic repeats (CRISPR) system, or a site-specific recombinase can be used to integrate a polynucleotide construct into the host cell genome, as described further below.

In some embodiments, a polynucleotide construct is integrated into the genome using a transposon system comprising a transposase and transposon donor DNA. The transposase can be provided to a host cell with an expression vector or mRNA comprising a coding sequence encoding the transposase. The transposon donor DNA can be provided with a vector comprising transposon terminal inverted repeats (TIRs). The polynucleotide construct is cloned into the transposon donor vector between the TIRs. The host cell is cotransfected with an expression vector or mRNA encoding the transposase and the transposon donor vector containing the polynucleotide construct insert, wherein the polynucleotide construct is excised from the transposon donor vector and integrated into the genome of the host cell at a target transposon insertion site. Transposition efficiency may be improved in a host cell by codon optimization of the transposase, using engineered hyperactive transposases, and/or introduction of mutations in the transposon terminal repeats. Any suitable transposon system can be used including, without limitation, the piggyBac, Tol2, or Sleeping Beauty transposon systems. For a description of various transposon systems, see, e.g., Kawakami et al. (2007) Genome Biol. 8 Suppl 1 (Suppl 1): S7, Tipanee et al. (2017) Biosci Rep. 37 (6): BSR20160614, Yoshida et al. (2017) Sci Rep. 7:43613, Yusa et al. (2011) Proc. Natl. Acad. Sci. USA 108 (4): 1531-1536, Doherty et al. (2012) Hum. Gene Ther. 23 (3): 311-320; herein incorporated by reference in their entireties.

In some embodiments, a construct is integrated at a target chromosomal locus by homologous recombination using site-specific nucleases or site-specific recombinases. For example, a construct can be integrated into a double-strand DNA break at the target chromosomal site by homology-directed repair. A DNA break may be created by a site-specific nuclease, such as, but not limited to, a Cas nuclease (e.g., Cas9, Cpf1, or C2c1), an engineered RNA-guided FokI nuclease, a zinc finger nuclease (ZFN), a transcription activator-like effector-based nuclease (TALEN), a restriction endonuclease, a meganuclease, a homing endonuclease, and the like. Any site-specific nuclease that selectively cleaves a sequence at the target site for integration of the construct may be used. See, e.g., Targeted Genome Editing Using Site-Specific Nucleases: ZFNs, TALENs, and the CRISPR/Cas9 System (T. Yamamoto ed., Springer, 2015); Genome Editing: The Next Step in Gene Therapy (Advances in Experimental Medicine and Biology, T. Cathomen, M. Hirsch, and M. Porteus eds., Springer, 2016); Aachen Press Genome Editing (CreateSpace Independent Publishing Platform, 2015); herein incorporated by reference in their entireties.

The construct sequence to be integrated is flanked by a pair of homology arms responsible for targeting the construct to the target chromosomal locus. A 5′ homology arm that hybridizes to a 5′ genomic target sequence and a 3′ homology arm that hybridizes to a 3′ genomic target sequence can be introduced into a polynucleotide construct. The homology arms are referred to herein as 5′ and 3′ (i.e., upstream and downstream) homology arms, which relates to the relative position of the homology arms in the polynucleotide construct. The 5′ and 3′ homology arms hybridize to regions within the target locus where the construct is integrated, which are referred to herein as the “5′ target sequence” and “3′ target sequence,” respectively.

The homology arm must be sufficiently complementary for hybridization to the target sequence to mediate homologous recombination between the construct and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein construct is integrated into the genomic DNA by HDR at the genomic target locus recognized (i.e., sufficiently complementary for hybridization) by the 5′ and 3′ homology arms.

In certain embodiments, the corresponding homologous nucleotide sequences in the genomic target sequence (i.e., the “5′ target sequence” and “3′ target sequence”) flank a specific site for cleavage and/or a specific site for integrating the construct. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate.

A homology arm can be of any length, e.g., 10 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another, e.g., one may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g., one may be 40% shorter or more, 50% shorter or more, sometimes 60% shorter or more, 70% shorter or more, 80% shorter or more, 90% shorter or more, or 95% shorter or more than the other homology arm.

An RNA-guided nuclease can be targeted to a particular genomic sequence (i.e., genomic target sequence for insertion of a polynucleotide construct) by altering its guide RNA sequence. A target-specific guide RNA comprises a nucleotide sequence that is complementary to a genomic target sequence, and thereby mediates binding of the nuclease-gRNA complex by hybridization at the target site. For example, the gRNA can be designed selectively bind to the chromosomal target site where integration of the construct is desired. In certain embodiments, the RNA-guided nuclease used for genome modification is a clustered regularly interspersed short palindromic repeats (CRISPR) system Cas nuclease. Any RNA-guided Cas nuclease capable of catalyzing site-directed cleavage of DNA to allow integration of polynucleotide constructs by the HDR mechanism can be used for selective integration at a target chromosomal site, including CRISPR system type I, type II, or type III Cas nucleases. Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

In certain embodiments, a type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to selectively integrate a construct at a chromosomal target site as described herein. The Cas9 need not be physically derived from an organism, but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP_002989955, WP_038434062, WP_011528583); Campylobacter jejuni (WP_022552435, YP_002344900), Campylobacter coli (WP_060786116); Campylobacter fetus (WP_059434633); Corynebacterium ulcerans (NC_015683, NC_017317); Corynebacterium diphtheria (NC_016782, NC_016786); Enterococcus faecalis (WP_033919308); Spiroplasma syrphidicola (NC_021284); Prevotella intermedia (NC_017861); Spiroplasma taiwanense (NC_021846); Streptococcus iniae (NC_021314); Belliella baltica (NC_018010); Psychroflexus torquisI (NC_018721); Streptococcus thermophilus (YP_820832), Streptococcus mutans (WP_061046374, WP_024786433); Listeria innocua (NP_472073); Listeria monocytogenes (WP_061665472); Legionella pneumophila (WP_062726656); Staphylococcus aureus (WP_001573634); Francisella tularensis (WP_032729892, WP_014548420), Enterococcus faecalis (WP_033919308); Lactobacillus rhamnosus (WP_048482595, WP_032965177); and Neisseria meningitidis (WP_061704949, YP_002342100); all of which sequences (as entered by the date of filing of this application) are herein incorporated by reference. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42 (4): 2577-90; Kapitonov et al. (2015) J. Bacteriol. 198 (5): 797-807, Shmakov et al. (2015) Mol. Cell. 60 (3): 385-397, and Chylinski et al. (2014) Nucleic Acids Res. 42 (10): 6091-6105); for sequence comparisons and a discussion of genetic diversity and phylogenetic analysis of Cas9.

The CRISPR-Cas system naturally occurs in bacteria and archaea where it plays a role in RNA-mediated adaptive immunity against foreign DNA. The bacterial type II CRISPR system uses the endonuclease, Cas9, which forms a complex with a guide RNA (gRNA) that specifically hybridizes to a complementary genomic target sequence, where the Cas9 endonuclease catalyzes cleavage to produce a double-stranded break. Targeting of Cas9 typically further relies on the presence of a 5′ protospacer-adjacent motif (PAM) in the DNA at or near the gRNA-binding site.

The genomic target site may comprise a nucleotide sequence that is complementary to the gRNA, and may further comprise a protospacer adjacent motif (PAM). In certain embodiments, the target site comprises 20-30 base pairs in addition to a 3 base pair PAM. Typically, the first nucleotide of a PAM can be any nucleotide, while the two other nucleotides will depend on the specific Cas9 protein that is chosen. Exemplary PAM sequences are known to those of skill in the art and include, without limitation, NNG, NGN, NAG, and NGG, wherein N represents any nucleotide. In certain embodiments, the allele targeted by a gRNA comprises a mutation that creates a PAM within the allele, wherein the PAM promotes binding of the Cas9-gRNA complex to the allele.

In certain embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1) may be used. Cpf1 is another class II CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571): 17-17, Zetsche et al. (2015) Cell. 163 (3): 759-771, Murovec et al. (2017) Plant Biotechnol. J. 15 (8): 917-926, Zhang et al. (2017) Front. Plant Sci. 8:177, Fernandes et al. (2016) Postepy Biochem. 62 (3): 315-326; herein incorporated by reference.

C2c1 is another class II CRISPR/Cas system RNA-guided nuclease that may be used. C2c1, similarly to Cas9, depends on both a crRNA and tracrRNA for guidance to target sites. For a description of C2c1, see, e.g., Shmakov et al. (2015) Mol Cell. 60 (3): 385-397, Zhang et al. (2017) Front Plant Sci. 8:177; herein incorporated by reference.

In yet another embodiment, an engineered RNA-guided FokI nuclease may be used. RNA-guided FokI nucleases comprise fusions of inactive Cas9 (dCas9) and the FokI endonuclease (FokI-dCas9), wherein the dCas9 portion confers guide RNA-dependent targeting on FokI. For a description of engineered RNA-guided FokI nucleases, see, e.g., Havlicek et al. (2017) Mol. Ther. 25 (2): 342-355, Pan et al. (2016) Sci Rep. 6:35794, Tsai et al. (2014) Nat Biotechnol. 32 (6): 569-576; herein incorporated by reference.

The RNA-guided nuclease can be provided in the form of a protein, such as the nuclease complexed with a gRNA, or provided by a nucleic acid encoding the RNA-guided nuclease, such as an RNA (e.g., messenger RNA) or DNA (expression vector) that is introduced into the host cell. Codon usage may be optimized to improve production of an RNA-guided nuclease in a particular cell or organism. For example, a nucleic acid encoding an RNA-guided nuclease can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease is introduced into cells, the protein can be transiently, conditionally, or constitutively expressed in the cell.

In some embodiments, a polynucleotide construct is site-specifically integrated into the genome of a host cell using a clustered regularly interspaced short palindromic repeats (CRISPR)-Cas9 system, wherein the construct is integrated into a Cas9-induced double-strand break at the target chromosomal site. A vector encoding Cas9 and a gRNA targeting the desired chromosomal site for integration is introduced into the host cell. Sequences with homology to the target locus are introduced into the polynucleotide construct to allow for integration by homology-directed repair. For a description of the use of a CRISPR-Cas9 systems for targeted genomic integration of AAV constructs, see, e.g., Nat. Commun. (2019) 10 (1): 4439; herein incorporated by reference.

Alternatively, site-specific recombinases can be used to selectively integrate a polynucleotide construct at a target chromosomal site. A target chromosomal site for integration of one or more polynucleotide constructs disclosed herein may include a transcriptionally active chromosomal sites. Examples of transcriptionally active chromosomal sites include DNaseI hypersensitive sites (DHSs). A polynucleotide construct can be site-specifically integrated into the genome of a host cell by introducing a first recombination site into the construct and expressing a site-specific recombinase in the host cell. The target chromosomal site of the host cell comprises a second recombination site, wherein recombination between the first and second recombination sites mediated by the site-specific recombinase results in integration of the vector at the target chromosomal locus. The target chromosomal site may comprise either a recombination site native to the genome of the host cell or an engineered recombination site recognized by the site-specific recombinase. Various recombinases may be used for site-specific integration of vector constructs, including, but not limited to phi C31 phage recombinase, TP901-1 phage recombinase, and R4 phage recombinase. In some cases, a recombinase engineered to improve the efficiency of genomic integration at the target chromosomal site may be used. For a description of various site-specific recombinase systems and their use in site-specific recombination and genomic integration of constructs, see, e.g., U.S. Pat. No. 6,632,672; Olivares et al. (2001) Gene 278:167-176; Stoll et al. (2002) J. Bacteriol. 184 (13): 3657-3663; Thyagarajan et al. (2001) Mol. Cell Biol. 21 (12): 3926-3934; Sclimenti et al. (2001) Nucleic Acids Res. 29 (24): 5044-5051; Stark et al. (2011) Biochem. Soc. Trans. 39 (2): 617-22; Olorunniji et al. (2016) Biochem. J. 473 (6): 673-684; Birling et al. (2009) Methods Mol. Biol. 561:245-63; García-Otin et al. (2006) Front. Biosci. 11:1108-1136; Weasner et al. (2017) Methods Mol. Biol. 1642:195-209; herein incorporated by reference in their entireties).

In some embodiments, one or more of the polynucleotide constructs are not integrated into the genome of the production host cell, and instead are maintained in the cell extrachromosomally. Examples of extrachromosomal polynucleotide constructs include those that persist as stable/persistent plasmids or episomal plasmids. In some embodiments, a construct comprises Epstein-Barr virus (EBV) sequences, including the EBV origin of replication. oriP, and the EBV gene, EBNA1, to provide stable extrachromosomal maintenance and replication of the construct. For a description of methods of using EBV sequences to stably maintain vectors extrachromosomally, see, e.g., Stoll et al. (2010) Mol. Ther. 4 (2): 122-129 and Deutsch et al. (2010) J. Virol. 84 (5): 2533-2546; herein incorporated by reference in their entireties. In some embodiments, the polynucleotide constructs of the present disclosure may be introduced into a cell in manner similar to the currently used triple-transfection method for production of rAAV virions.

In a preferred embodiment, this system requires only one antibiotic resistance marker, and two split auxotrophic constructs for selection of all three plasmids, each being transformed just once into the DHFR knockout strain-producing a master cell line for virion production which can be stored and then utilized for scaled-up production without further transformations. This approach provides inducible control over expression of the Rep/Cap products avoiding the toxicity typically associated with Rep/Cap production and also avoids selection with multiple antibiotics, which is not preferred for therapeutic products. Both overexpression of Rep/Cap and selection with multiple antibiotics can be toxic and result in diminished virion yield. The transformed cells can be frozen for storage and thawed for subsequent applications.

### Payloads

Disclosed herein are payloads that may be encoded for by polynucleotide construct 3, which encodes for a payload. This third polynucleotide is referred to herein as a “payload construct” or “therapeutic payload.” Thus, disclosed herein are stable mammalian cell lines that encapsidate a payload. The payload may be an expressible payload. The polynucleotide may encode for any therapeutic. For example, the therapeutic may be a transgene, a guide RNA, an antisense RNA, an oligonucleotide, an mRNA, a miRNA, a shRNA, a tRNA suppressor, a CRISPR-Cas protein, any gene editing enzyme, or any combination thereof. In some embodiments, the payload is guide RNA, wherein the guide RNA, when bound to a target RNA, recruits an ADAR enzyme for editing of the target RNA. In some embodiments, the payload is progranulin. In some embodiments, any one of SEQ ID NO: 146-SEQ ID NO: 153 comprise the progranulin payload. In some embodiments, the payload is progranulin and is flanked by ITRs, e.g., is an ITR flanked progranulin payload of SEQ ID NO: 146 or SEQ ID NO: 147. In some embodiments, the ITR flanked progranulin payload has at least 70%, 75%, 80%, 85%, 90%, 95%, 96%, 97%, 98%, 99%, or 100% sequence identity to the ITR flanked progranulin payload of any one of SEQ ID NO: 146-SEQ ID NO: 153. In some embodiments, the stable mammalian cell lines disclosed herein can conditionally produce rAAV virions that encapsidate more than one payload. Any combination of payloads disclosed herein is contemplated.

Production of Single-Stranded or Self-Complementary rAAV Virion DNA

The region of the third polynucleotide construct between the two inverted terminal repeats (3′ ITR and 5′ ITR) is packaged into rAAV virions. In some embodiments, the rAAV virions comprise wild-type inverted terminal repeats, wherein the rAAV virion DNA that is generated is single-stranded (i.e., ssAAV virion). In other embodiments, a terminal resolution site in the 3′ ITR is deleted, resulting in formation of an rAAV virion comprising DNA that is self-complementary (i.e., scAAV virion). The scAAV forms a single-stranded DNA molecule during replication in which two single-stranded genomes comprising a plus DNA strand and a minus DNA strand are concatenated to form a self-complementary intramolecular dsDNA genome. Unlike ssAAV virions, the scAAV virions do not need to perform second-strand DNA synthesis, which increases the efficiency of scAAV transgene expression relative to ssAAV. However, the maximum cargo capacity of scAAV (i.e., maximum length of region between the 5′ ITR and 3′ ITR of the third polynucleotide construct) that can be packaged into the rAAV virion is about half that of ssAAV because the scAAV DNA packaged into a viral particle is a concatemer of two single-stranded genomes of opposite strands. For a description of methods of producing scAAV virions, see, e.g., Raj et al. (2011) Expert Rev. Hematol. 4 (5): 539-549, McCarty (2008) Mol. Ther. 16 (10): 1648-1656, McCarty et al. (2003) Gene Ther. 10 (26): 2112-2118; herein incorporated by reference in their entireties.

### Split Auxotrophic Selection

Maintaining constructs stably in the cellular genome requires selective pressure.

Typically, each integrated nucleic acid construct comprises a mammalian cell selection element. In some embodiments, the stable cell line comprises three integrated nucleic acid constructs, wherein the first nucleic acid construct comprises a first mammalian cell selection element, the second nucleic acid construct comprises a second mammalian cell selection element, and the third nucleic acid construct comprises a third mammalian cell selection element.

In some embodiments, the mammalian selection elements are components of a split auxotrophic selection system. In some embodiments, a first mammalian selection element, a second mammalian selection element, or a third mammalian selection element can be a component of a split auxotrophic selection system. For example, a first mammalian selection element can be a first component of the split auxotrophic selection system and second mammalian selection element can be a second component of the split auxotrophic selection system. As another example, a second mammalian selection element can be a first component of the split auxotrophic selection system and third mammalian selection element can be a second component of the split auxotrophic selection system.

A split auxotrophic system can be a leucine zipper based system that permits stable retention of two integrated nucleic acid constructs under a single selective pressure. In some embodiments, components of the split auxotrophic selection system described herein comprise a C-terminal fragment of the auxotrophic protein Z-Cter and an N-terminal fragment of an auxotrophic protein Z-Nter. In some embodiments, a split auxotrophic system is a split intervening proteins (inteins) system that permits stable retention of two integrated nucleic acid constructs under a single selective pressure. Inteins auto catalyze a protein splicing reaction that results in excision of the intein and joining of the flanking amino acids (extein sequences) via a peptide bond. Inteins exist in nature as a single domain within a host protein or, less frequently, in a split form. For split inteins, the two separate polypeptide fragments of the intein must associate in order for protein trans-splicing to occur to excise the intein. Split intein systems are described in: Cheriyan et al, J. Biol. Chem 288:6202-6211 (2013); Stevens et al, PNAS 114:8538-8543 (2017); Jillette et al., Nat Comm 10:4968 (2019); US 2020/0087388 A1; and US 2020/0263197 A1. In some embodiments, components of the split auxotrophic selection system described herein comprises a construct encoding an N-terminal fragment of an auxotrophic protein fused to an N-terminal intein of the split intein and a construct encoding the C-terminal fragment of the auxotrophic protein fused to a C-terminal intein of the split intein. This N-terminal fragment is enzymatically nonfunctional and this C-terminal fragment is enzymatically nonfunctional. When both fragments are concurrently expressed in the cell, the split inteins can catalyze the joining of the N-terminal fragment of the auxotrophic protein and a C-terminal fragment of the auxotrophic protein to form a functional enzyme, such as any one of the enzymes disclosed herein (e.g., PAH, GS, TYMS, DHFR). In some embodiments, both constructs can be stably retained in the genome of a cell by growth in a medium lacking the product produced by the enzyme.

In some embodiments, a construct encoding for a component of a split auxotrophic system further encodes a helper enzyme, wherein expression of the helper enzyme facilitates growth of the host cell in conjunction with the functional enzyme upon application of the single selective pressure.

FIG. 5A depicts an exemplary split auxotrophic selection system that permits stable retention of two integrated nucleic acid constructs under a single selective pressure. One construct encodes the N-terminal fragment of mammalian dihydrofolate reductase (DHFR) fused to a leucine zipper peptide (“Nter-DHFR”). This N-terminal fragment is enzymatically nonfunctional. The other construct encodes the C-terminal fragment of DHFR fused to a leucine zipper peptide (“Cter-DHFR”). This C-terminal fragment is enzymatically nonfunctional. When both fragments are concurrently expressed in the cell, a functional DHFR enzyme complex is formed through association of the leucine zipper peptides. Both constructs can be stably retained in the genome of a DHFR null cell by growth in a medium lacking hypoxanthine and thymidine.

FIG. 5B shows an exemplary deployment of this split auxotrophic selection design in the multi-construct system of FIG. 1 in its pre-triggered state. In this embodiment, the split auxotrophic selection elements are deployed on constructs 1 and 3. A separate exemplary antibiotic selection element, blasticidin resistance, is deployed on construct 2. This results in the ability to stably maintain all three constructs in the mammalian cell line using a single antibiotic, culturing in medium with blasticidin, lacking thymidine and hypoxanthine. In some embodiments, the construct 2 further comprising a sequence coding for VA RNA as described herein. In some embodiments, the VA RNA is a mutated VA RNA. In some embodiments, the VA RNA is transcriptionally dead VA RNA. In some embodiments, the VA RNA is under the control of a U6 promoter. In some embodiments, the U6 promoter is a conditionally active. In some embodiments, the U6 promoter comprises an interrupting sequence that is capable of being floxed upon addition a triggering agent (e.g., the triggering agent induces the expression of a recombinase as described herein).

In some embodiments, the first nucleic acid construct comprises a first mammalian cell selection element, and the first mammalian cell selection element is a first auxotrophic selection element. In certain embodiments, the first auxotrophic selection element encodes an active protein. In some embodiments, the first auxotrophic selection element is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the first auxotrophic selection element codes for an inactive protein that requires expression of a second auxotrophic selection element for activity. In some embodiments, the first auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the first auxotrophic selection element codes for DHFR Z-Cter or DHFR Z-Nter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the first auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the first auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the first auxotrophic selection element codes for C-terminal fragment of PAH, GS, TYMS, or DHFR fused to a C-terminal intein of a split intein. In some embodiments, the first auxotrophic selection element codes for an N-terminal fragment of PAH, GS, TYMS, or DHFR fused to a N-terminal intein of a split intein. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, first nucleic acid construct further comprises a sequence coding for a first auxotrophic selection element and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the first nucleic acid construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a first auxotrophic selection element comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the first auxotrophic selection element and helper enzyme comprises at least 80%, 85%, 90%, 95%, 98%, 99%, or 100% sequence identity to SEQ ID NO: 101-SEQ ID NO: 109.

In various embodiments, the second nucleic acid construct comprises a second mammalian cell selection element, and the second mammalian cell selection element encodes antibiotic resistance. In particular embodiments, the antibiotic resistance gene is a blasticidin resistance gene. In certain embodiments, the second mammalian cell selection element encodes an active protein. In some embodiments, t the second mammalian cell selection element is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123.

In various embodiments, the third nucleic acid construct comprises a third mammalian cell selection element. In some embodiments, the third mammalian cell selection element is a second auxotrophic selection element. In certain embodiments, the second auxotrophic selection element encodes an active protein. In some embodiments, the second auxotrophic selection element is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the second auxotrophic selection element codes for an inactive protein that requires expression of a first auxotrophic selection element for activity. In some embodiments, the second auxotrophic selection element codes for a C-terminal fragment of the auxotrophic protein Z-Cter and the first auxotrophic selection element codes for N-terminal fragment of an auxotrophic protein Z-Nter, or vice a versa. In some embodiments, the second auxotrophic selection element codes for DHFR Z-Cter or DHFR Z-Nter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the second auxotrophic selection element codes for a C-terminal fragment of the auxotrophic protein fused to a C-terminal intein of a split intein and the first auxotrophic selection element codes for an N-terminal fragment of an auxotrophic protein fused to an N-terminal intein of a split intein. In some embodiments, the second auxotrophic selection element codes for an N-terminal fragment of an auxotrophic protein fused to an N-terminal intein of a split intein and the first auxotrophic selection element codes for a C-terminal fragment of the auxotrophic protein fused to a C-terminal intein of a split intein. In some embodiments, the second auxotrophic selection element codes for C-terminal fragment of PAH, GS, TYMS, or DHFR fused to a C-terminal intein of a split intein. In some embodiments, the second auxotrophic selection element codes for an N-terminal fragment of PAH, GS, TYMS, or DHFR fused to a N-terminal intein of a split intein. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, third nucleic acid construct further comprises a sequence coding for second auxotrophic element and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the third nucleic acid construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a second auxotrophic selection element comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the second auxotrophic selection element and helper enzyme comprises at least 80%, 85%, 90%, 95%, 98%, 99%, or 100% sequence identity to SEQ ID NO: 101-SEQ ID NO: 109.

In various embodiments, the selectable marker of the first nucleic acid construct, the selectable marker of the second nucleic acid construct, and the selectable marker of the third nucleic acid construct are interchangeable between each other. For example, the first nucleic acid construct can comprise the second auxotrophic selection element, the second nucleic acid construct can comprise the first auxotrophic selection element, and the third nucleic acid construct can comprise the mammalian selection element.

In various embodiments, the stable mammalian cell line can be propagated in growth media lacking hypoxanthine and thymidine.

### Exemplary Complete System in Detail

Described herein are many different combinations of elements in constructs, in combination, are capable of conditionally producing AAV virion in a cell. An exemplary complete system is described as follows, which is a non-limiting example. It is understood that variations of the elements described herein can be used in any of constructs described below, and the elements of the constructs described below may be split into one or more separate constructs. The first integrated synthetic construct comprises an intervening spacer sequence inserted into the coding sequence of AAV2 Rep protein. The intervening spacer sequence comprises an enhanced green fluorescent protein (EGFP) and a rabbit beta globin (RBG) polyadenylation (polyA) signal, flanked by two lox sites, are inserted into an RBG intron. The RBG intron includes the 5′ splice site (5′SS) and the 3′ splice site (3′SS) (as shown in FIGS. 3A-3B). The RBG intron is inserted downstream of the Rep endogenous P5 and P19 promoters and interrupt the Rep coding sequence. This design blocks the expression of Rep proteins generated by both P5 and P19 promoters. The EGFP serves as a visual indicator of successful integration and to monitor Cre mediated excision, and could be replaced by any suitable marker. For instance, loss of EGFP expression indicates successful Cre-mediated genomic recombination (See, FIG. 3B). Current approaches rely on inserting the EGFP and the poly A within an intron without duplication of 3′ splice site (3′SS). If there is readthrough after polyA, the 5′SS can combine with the native 3′SS, thus removing the entire RBG intron and as a result, commencing Rep expression. By contrast, the design as described herein includes an additional 3′SS upstream of the EGFP, which solves the problem of undesired Rep expression. The present design provides that if there is readthrough, the construct allows splicing of 5′SS to the upstream 3′SS. Without being bound to any theories, the additional 3′SS which is nearest to the 5′SS is preferred since it is the same 3′SS as the downstream one and the two 3′SS are of equal strength. As such, all Rep proteins will be produced fused to the EGFP protein and then terminate. In an event where the Rep proteins do not terminate after EGFP, they will continue to produce codons coded by the rest of the RBG intro, thereby making a non-functional Rep protein. This approach prevents overexpression of Rep proteins which may have inhibitory effects on adenovirus and cell growth, thereby reducing toxicity of the recombinant AAV (rAAV) construct.

As described herein, expression of functional Rep protein is induced only in the presence of a first expression triggering agent, e.g., the addition of doxycycline which results in the production of Cre. In the presence of Cre, the intervening spacer is excised thereby resuming intact coding sequencing of the Rep protein. This approach provides controlled and inducible Rep expression.

This is driven by the second integrated synthetic construct, which comprises an estrogen inducible Cre (ER2 Cre) gene and adenoviral helper genes, E2A and E4orf6 (E4) (See, FIGS. 1, 2A-2B, 3A-3B, and 6).

In certain embodiments, the third integrated synthetic construct (“construct three”) comprises a polynucleotide flanked by AAV inverted terminal repeats (ITRs, shown by brackets in FIG. 1, FIG. 4, FIG. 5B, and FIG. 6). In certain embodiments, the third integrated construct further comprises a component of a split auxotrophic selection, as described above in Section 4.4.5. In particular embodiments, the component of the split auxotrophic selection comprises a first enzymatically nonfunctional dihydrofolate reductase (DHFR) fragment fused to a leucine zipper. Binding with a second DHFR fragment also fused to a leucine zipper produces an active complex, and allows selection for cells expressing both the first and the third integrated synthetic constructs. The construct three polynucleotide can comprise any payload including at least a guide RNA, a gene of interest, a transgene, an HDR homology region, a minigene or a therapeutic polynucleotide. This approach requires only a single auxotrophic selection agent, and a single antibiotic selection agent to be present in the cell culture medium to maintain all of the plurality of synthetic nucleic acid constructs stably within the nuclear genome of the cells. This approach also avoids multiple antibiotic resistance selection, which may be undesirable for downstream applications, e.g., gene therapy.

In one aspect, provided herein is a stable mammalian cell line, wherein the cells are capable of conditionally producing recombinant AAV (rAAV) virions within which are packaged an expressible payload; and production of virions is not conditioned on the presence of an episome within the cell.

In various embodiments, expression of AAV rep and cap proteins is conditional. In some embodiments, expression of AAV rep and cap proteins is conditioned on addition of at least a first expression triggering agent to the cell culture medium. In some embodiments, expression of AAV Rep and Cap proteins is conditioned on addition of a first expression triggering agent and a second expression triggering agent to the cell culture medium.

In some embodiments, the cells do not express cytotoxic levels of Rep protein prior to addition of the at least a first expression triggering agent to the cell culture medium. In some embodiments, the cells do not express cytostatic levels of Rep protein prior to addition of the at least first expression triggering agent to the cell culture medium.

In some embodiments, the average concentration of Rep protein within the cells is less than between 1-99%, 10-90%, 20-80%, 30-70%, 40-60% prior to addition of the at least first expression triggering agent to the cell culture medium. In some embodiments, the average concentration of Rep protein within the cells is less than about 1%, 5%, 10%, 15%, 20%, 25%, 30%, 35%, 40%, 45%, 50%, 55%, 60%, 65%, 70%, 75%, 80%, 85%, 90%, 95%, or 99% prior to addition of the at least first expression triggering agent to the cell culture medium.

In various embodiments, expression of Rep and Cap proteins becomes constitutive after addition of the at least first expression triggering agent to the cell culture medium. The stable cell lines include those wherein expression of adenoviral helper proteins is conditional. In some embodiments, expression of adenoviral helper proteins is conditioned on addition of at least a first expression triggering agent to the cell culture medium. In some embodiments, expression of adenoviral helper proteins is conditioned on addition of a first expression triggering agent and a second expression triggering agent to the cell culture medium. In some embodiments, continued expression of adenoviral helper proteins following triggering of expression requires presence of only the first expression triggering agent in the cell culture medium.

In some embodiments, the adenoviral helper proteins include E2A and E4.

In some embodiments, the first expression triggering agent is a tetracycline. In some embodiments, the tetracycline is doxycycline.

In some embodiments, the second expression triggering agent is an estrogen receptor ligand. In some embodiments, the estrogen receptor ligand is a selective estrogen receptor modulator (SERM). In some embodiments, the estrogen receptor ligand is tamoxifen.

In some embodiments, expression of the payload is not conditioned on addition of an expression triggering agent to the cell culture medium.

In various embodiments, the nuclear genome of the cell comprises a plurality of integrated synthetic nucleic acid constructs. In some embodiments, the nuclear genome of the cell comprises two integrated synthetic constructs. In some embodiments, the nuclear genome of the cell comprises three integrated synthetic constructs. In some embodiments, each of the plurality of synthetic nucleic acid constructs is separately integrated into the nuclear genome of the cell.

In some embodiments, only a single non-auxotrophic selection agent is required to be present in the cell culture medium to maintain all of the plurality of synthetic nucleic acid constructs stably within the nuclear genome of the cells.

In some embodiments, the first integrated synthetic construct comprises conditionally expressible AAV Rep and Cap coding sequences; the second integrated synthetic construct comprises a conditionally expressible Cre coding sequence and conditionally expressible adenoviral helper protein coding sequences; and the third integrated synthetic construct comprises expressible coding sequences for the payload.

In some embodiments, the first integrated construct comprises a Rep coding sequence interrupted by an intervening spacer. In some embodiments, the intervening spacer comprises, from 5′ to 3′, a first spacer, a second spacer and a third spacer. In some embodiments, the intervening spacer comprises nucleic acid sequences of a rabbit beta globin (RBG) intron and a rabbit beta globin (RBG) poly A. In some embodiments, the first spacer comprises a nucleic acid sequence of at least 80% identity to SEQ ID NO: 1. In some embodiments, the first spacer comprises a 5′ splice site (5′SS) 5′ to the first spacer. In some embodiments, the second spacer comprises a nucleic acid sequence of at least 80% identity to SEQ ID NO: 2. In some embodiments, the second spacer comprises, from 5′ to 3′ a first lox site, an enhanced green fluorescent protein (EGFP), the RBG polyA sequence, and a second lox site. In some embodiments, the second spacer further comprises a first 3′ splice site (3′SS) flanked by the first lox site and the EGFP. In some embodiments, the third spacer comprises a nucleic acid sequence of at least 80% identity to SEQ ID NO: 3. In some embodiments, the third spacer further comprises a second 3′ splice site (3′SS) 3′ to the third spacer.

In some embodiments, the Rep coding sequence comprises a polynucleotide sequence operatively linked to an endogenous P5 promoter. In some embodiments, the Rep coding sequence comprises a polynucleotide sequence operatively linked to an endogenous P19 promoter. In some embodiments, the intervening spacer is inserted into the Rep coding sequence at a position downstream of the P19 promoter. In some embodiments, the intervening spacer is inserted into the Rep coding sequence at a position in frame with the protein produced from activation of the P5 promoter and the P19 promoter. In some embodiments, wherein the Rep coding sequence is 5′ to the Cap coding sequence. In some embodiments, the Cap coding sequence is operatively linked to an endogenous P40 promoter.

In some embodiments, the second integrated construct comprises, from 5′ to 3′, a Cre coding sequence and a first polyA sequence, adenoviral helper protein coding sequences and a second polyA sequence, a first expression triggering agent responsive element, and an antibiotic selection element. In some embodiments, the Cre coding sequence is flanked by a first lox site and a second lox site. In some embodiments, the Cre coding sequence is operatively linked to an inducible promoter. In some embodiments, the inducible promoter comprises a plurality of tetracycline (Tet) operator elements capable of binding to a Tet responsive activator protein in the presence of a first expression triggering agent. In some embodiments, the inducible promoter comprises a plurality of Tet operator elements capable of binding to a Tet responsive activator protein in the presence of a first expression triggering agent and a second expression triggering agent responsive element. In some embodiments, the adenoviral helper protein coding sequences comprise E2A and E4 sequences. In some embodiments, the first expression triggering agent responsive element is operatively linked to a CMV promoter. In some embodiments, the first expression triggering agent responsive element comprises the Tet responsive activator protein (Tet-on-3G). In some embodiments, the antibiotic selection element is blasticidin resistance.

In some embodiments, the third integrated synthetic construct comprises a coding sequence for the expressible payload and a first element of an auxotrophic selection agent and the first integrated synthetic construct comprises coding sequences for a second element of the auxotrophic selection agent. In some embodiments, the first element of a auxotrophic selection agent comprises a first dihydrofolate reductase (DHFR) selectable marker (SEQ ID NO: 4). In some embodiments, the first DHFR comprises a leucine zipper (Nter). In some embodiments, the second element of the auxotrophic selection agent comprises a second DHFR (SEQ ID NO: 5). In some embodiments, the second DHFR comprises a leucine zipper (Cter). In some embodiments, the DHFR selection comprises the ability to grow in media lacking hypoxantine-thymidine.

In some embodiments, the mammalian cell line is selected from the group consisting of a human embryonic kidney (HEK) 293 cell line, a human HeLa cell line, and a Chinese hamster ovary (CHO) cell line. In some embodiments, the mammalian cell line is a HEK293 cell line. In some embodiments, the mammalian cell line expresses adenovirus helper functions E1A and E1B.

Alternative constructs as described herein can be used in a complete system. The complete system can be integrated into the host cell genome to produce a stable cell line. The complete system can be transfected into the host cell and then conditional production of AAV virion from the plasmids can be induced. In some embodiments, the complete system comprises episomes in the host cell and conditional production of AAV virion from the episomes is induced. In some embodiments, a complete system that lacks the elements of a tetracycline-inducible system is induced by adding tamoxifen to cause translocation of the Cre to the nucleus and subsequent production of AAV virion.

### A. The Stable Mammalian Cell or Cell Line

As described herein, the stable mammalian cell or cell line can be a human derived cell or cell line such as a human embryonic kidney (HEK) 293 cell line or a human HeLa cell line, or a mammalian cell or cell line such as Chinese hamster ovary (CHO) cell line. In some embodiments, the mammalian cell line is a HEK293 cell line. In some embodiments, the mammalian cell line expresses adenovirus helper functions E1A and E1B. In some embodiments, the mammalian cell line expresses adenovirus helper functions E2A and E4 (e.g., E4orf6).

### B. The First Integrated Synthetic Construct

Shown are exemplary designs of the first integrated synthetic construct (FIGS. 1, 3B and 6).

As described in FIGS. 3A-3B, the first integrated synthetic construct comprises a Rep coding sequence 5′ of a Cap coding sequence. The Rep coding sequence is interrupted by an intervening spacer. In some embodiments, the first integrated synthetic construct further comprises a selection element such as an auxotrophic selection marker (FIG. 5B). In some embodiments, the selection element is a partial or a second element of the non-auxotrophic selection marker. The intervening spacer comprises a rabbit beta globin (RBG) intron, which is modified by duplicating the RBG 3′ splice site (3′SS) upstream to an enhanced green fluorescent protein (EGFP) cassette within the intron. The EGFP cassette is cloned immediately downstream of this duplicated splice site followed by a rabbit beta globin polyadenylation signal. This entire modification (3′SS, EGFP and polyA) is flanked by two lox sites so that the module can be removed upon Cre expression. The modified rabbit beta globin intron (the intervening spacer sequence) is inserted into the coding sequence of AAV2 Rep protein. The point of insertion is downstream of P19 promoter, away from any known regulatory elements. It is also in frame with the proteins produced from P5 and P19 protein so that EGFP expression can be visualized. Cap genes from any AAV serotype are cloned downstream of the AAV2 Rep cassette. The Cap genes are driven by their endogenous P40 promoter. In the absence of Cre, the 5′ splice site (5′SS) gets spliced to the upstream of 3′SS, so the EGFP becomes the terminal exon and transcription terminates at the beta globin polyadenylation signal. Thus, the expression of all Rep proteins either from the P5 or P19 promoter is prematurely terminated. Since expression of the P40 promoter is dependent on the presence of the Rep proteins, the P40 promoter is silent and there is no expression of Cap proteins. Upon Cre expression from a second integrated synthetic construct, the entire second spacer element (except for the left lox site is excised from the beta globin intron. The 5′SS now splices with the native 3′SS site and expression of all Rep proteins commences. Rep expression activates the P40 proteins and Cap proteins are therefore also expressed.

Alternative Rep/Cap constructs as described herein can be integrated into a host cell genome to produce the stable cell line. Alternative Rep/Cap constructs that are non-integrating as described herein can be introduced into a host cell genome.

### C. The Second Integrated Synthetic Construct

Shown are exemplary designs of the second integrated synthetic construct (FIGS. 2A-2C).

As shown in FIG. 2A, the second integrated synthetic construct comprising, from 5′ to 3′, a Cre coding sequence and a first polyA sequence, adenoviral helper protein coding sequences and a second polyA sequence, a first expression triggering agent responsive element, and an antibiotic selection element. The Cre coding sequence is flanked by a first lox site and a second lox site, and is operatively linked to an inducible promoter. The inducible promoter comprises a plurality of tetracycline (Tet) operator elements capable of binding to a Tet responsive activator protein in the presence of a first expression triggering agent, e.g., doxycycline or tetracycline. In some embodiments, the inducible promoter comprises a plurality of tetracycline (Tet) operator elements capable of binding to a Tet responsive activator protein in the presence of a first expression triggering agent and a second expression triggering agent responsive element, e.g., tamoxifen. The first expression triggering element may comprise a Tet responsive activator protein (Tet-on-3G) and is operatively linked to a CMV promoter. The antibiotic selection element can be blasticidin resistance (FIG. 5B). The optional insert shown in FIG. 2C provides for inducible production of VA-RNA, which are short non-coding transcripts essential for Adenovirus replication. In this construct, an alternative insert to construct 2, includes a Cre inducible U6 promoter that drives the expression of transcriptionally dead mutants of VA RNA1 (a preferred embodiment is a double point mutant G16A-G60A). The U6 promoter is split into 2 parts separated by a Lox flanked stuffer sequence. The U6 promoter is inactive because of the presence of the stuffer sequence. Cre mediated excision of the stuffer activates the U6 promoter which then drives the expression of VA RNA. Other embodiments may provide for alternative sources of VA-RNA.

In some embodiments, the Cre coding sequencing is an estrogen inducible Cre that has a strong polyadenylation signal (stop signal) at its 3′ end. Following this is a bicistronic E2A, E4orf6 cassette. The plasmid also has a constitutive promoter (CMV) which drives the expression of the Tet responsive activator protein (Tet-on 3G).

In the off state when doxycycline (Dox) is absent, the Tet-on 3G cannot bind to the Tet operator elements in the Tet-regulatable promoter so the promoter is not active. Estrogen responsive Cre is used instead of simple Cre to counteract the basal or leaky expression of the Tet-regulatable promoter. In the off state if there is leaky expression of Cre gene, the expressed Cre protein will be held inactive in the cytoplasm. The strong polyadenylation signal, 3′ of the cre gene will prevent basal expression of adenoviral helper genes, E2A and E4. To induce expression, doxycycline and tamoxifen are added to the cell culture (FIG. 6). Doxycycline will bind to the Tet-on 3G protein and this will promote binding of the Tet-on 3G to the tet operator elements in the Tet-regulatable promoter. This will trigger the activation of the promoter. ER2 Cre will be expressed at high levels and tamoxifen will bring the Cre to the nucleus.

Alternative helper constructs as described herein can be integrated into a host cell genome to produce the stable cell line. Alternative helper constructs that are non-integrating as described herein can be introduced into a host cell genome.

### D. The Third Integrated Synthetic Construct

Shown are exemplary designs of the third integrated synthetic construct (FIGS. 1, 4, 5B and 6). As described in FIGS. 4 and 6, the third integrated synthetic construct comprises coding sequences for an expressible payload, and/or a guide RNA, and a first element of a non-auxotrophic selection agent capable of binding to a partial or a second element of the non-auxotrophic selection agent in the first integrated synthetic construct. In some embodiments, the first element of a non-auxotrophic selection agent comprises a first dihydrofolate reductase (DHFR) selectable marker (SEQ ID NO: 4). The first DHFR may comprise a leucine zipper (Nter). In some embodiments, the second element of the non-auxotrophic selection agent comprises a second DHFR (SEQ ID NO: 5). The second DHFR may comprise a leucine zipper (Cter). In some embodiments, the DHFR selection comprises hypoxantine-thymidine selection. In some embodiments, re-association of the first and second DHFR selection markers allows for selection of a mammalian cell expression both the first integrated synthetic construct and the third integrated synthetic construct.

Alternative payload constructs as described herein can be integrated into a host cell genome to produce the stable cell line. Alternative payload constructs that are non-integrating as described herein can be introduced into a host cell genome.

In some embodiments, a cell comprises two constructs (any combination of Rep/Cap construct, inducible helper construct, and the payload construct). In some embodiments, a cell comprises the Rep/Cap construct and the inducible helper construct. In some embodiments, the inducible helper construct comprises a VA RNA construct as described herein. In some embodiments, cell further comprises the VA RNA construct.

In some embodiments, a cell comprises any combination of Rep/Cap construct(s) and inducible helper construct(s) as described herein. In some embodiments, a cell comprises any combination of Rep/Cap construct(s) and payload construct(s) as described herein. In some embodiments, a cell comprises any combination of inducible helper construct(s) and payload construct(s) as described herein. In some embodiments, a cell comprises any combination of Rep/Cap construct(s), inducible helper construct(s), and the payload construct(s) as described herein that are capable of producing AAV virion upon induction. In some embodiments, a cell comprises the Rep/Cap construct(s) and the inducible helper construct(s) of the complete system capable of producing AAV virion upon induction. In some embodiments, a cell comprises the Rep/Cap construct(s) and the payload construct(s). In some embodiments, a cell comprises the payload construct(s) and the inducible helper construct(s) of the complete system capable of producing AAV virion upon induction. In some embodiments, a Rep/Cap construct, an inducible helper construct, or a payload construct further comprises a VA RNA construct as described herein. In some embodiments, the cell further comprises a separate VA RNA construct as described herein.

In some embodiments, a cell comprises all three constructs (Rep/Cap construct, inducible helper construct, and the payload construct). In some embodiments, a cell comprises any combination of Rep/Cap construct(s), inducible helper construct(s), and the payload construct(s) as described herein that are capable of producing AAV virion upon induction. In some embodiments, the inducible helper construct comprises a VA RNA construct as described herein. In some embodiments, a Rep/Cap construct, an inducible helper construct, or a payload construct further comprises a VA RNA construct as described herein. In some embodiments, cell further comprises the VA RNA construct. In some embodiments, this cell is capable of producing an rAAV virion upon addition of at least one triggering agent. In some embodiments, the rAAV virion comprising the capsid protein and the payload nucleic acid sequence have an infectivity of no less than 50%, 60%, 70%, 80%, 90%, 95%, or 99% at an MOI of 1×10⁵ vg/target cell or less. In some embodiments, the rAAV virions have an increased infectivity compared rAAV virions produced by an otherwise comparable the population of cells capable of producing rAAV virions upon transient transfection at the same MOI. In some embodiments, the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI. In some embodiments, the rAAV virions have at least 50%, 60%, 70%, 80%, 90%, 95%, 97%, 98%, 99%, or 100% infectivity as compared to AAV virions produced by a cell having wildtype AAV at the same MOI. In some embodiments, the rAAV virions have at least 50%, 60%, 70%, 80%, 90%, 95%, 97%, 98%, 99%, or 100% infectivity as compared to AAV virions at the same MOI. In some embodiments, the AAV virions are wildtype AAV virions produced by a cell having wildtype AAV. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the cell is conditionally capable of producing rAAV virions having a payload encapsidation ratio of no less than 0.5, 0.6, 0.7, 0.8, 0.9, 0.95, 0.97, or 0.99. In some embodiments, the rAAV virions have a payload encapsidation ratio of no less than 0.5, 0.6, 0.7, 0.8, 0.9, 0.95, 0.97, or 0.99 prior to purification. In some embodiments, the rAAV virions have a concentration of greater than 1×10¹¹ or no less than 5×10¹¹, 1×10¹², 5×10¹², 1×10¹³ or 1×10¹⁴ viral genomes per milliliter prior to purification. In some embodiments, the cell is capable of producing rAAV virions comprising the payload nucleic acid sequence at a titer of greater than 1× 10¹¹ or no less than 5×10¹¹, 1×10¹², 5×10¹², 1×10¹³ or 1×10¹⁴ viral genomes per milliliter. In some embodiments, the cell is capable of producing rAAV virions comprising the payload nucleic acid sequence at a concentration of greater than 1×10¹¹ or no less than 5×10¹¹, 1×10¹². 5×10¹², 1×10¹³ or 1×10¹⁴ viral genomes per milliliter prior to purification. In some embodiments, this cell is expanded to produce a population of cells. In some embodiments, the population of cells produces a stable cell line as described herein. In some embodiments, this cell is passaged at least three times. In some embodiments, this cell can be passaged up to 60 times. In some embodiments, this cell can be passage more than 60 times. In some embodiments, the cell maintains the ability to be conditionally induced after each passage.

### Cell Comprising a Construct

In some embodiments, a cell comprises one construct (Rep/Cap construct, inducible helper construct, and the payload construct). In some embodiments, the one construct is stably integrated into the genome of the cell. In some embodiments, the one construct is not stably integrated into the genome of the cell. In some embodiments, a plurality of the one construct is stably integrated into the genome of the cell. In some embodiments, a plurality of the one construct is not stably integrated into the genome of the cell. In some embodiments, a cell comprises two constructs (any combination of Rep/Cap construct, inducible helper construct, and the payload construct). In some embodiments, the two constructs are stably integrated into the genome of the cell. In some embodiments, the two constructs are not stably integrated into the genome of the cell. In some embodiments, the two constructs are separately stably integrated into the genome of the cell. In some embodiments, the two constructs are not separately stably integrated into the genome of the cell. In some embodiments, a plurality of the two constructs are stably integrated into the genome of the cell. In some embodiments, a plurality of the two constructs are not stably integrated into the genome of the cell. In some embodiments, a plurality of the two constructs are separately stably integrated into the genome of the cell. In some embodiments, a plurality of the two constructs are not separately stably integrated into the genome of the cell. In some embodiments, a cell comprises the Rep/Cap construct and the inducible helper construct. In some embodiments, a cell comprises the Rep/Cap construct(s) as disclosed herein, the inducible helper construct(s) as disclosed herein, payload construct(s) as disclosed herein, or any combination thereof. In some embodiments, the cell, the inducible helper construct comprises a VA RNA construct as described herein. In some embodiments, cell further comprises the VA RNA construct as described herein. In some embodiments, the VA RNA construct is stably integrated into the genome of the cell. In some embodiments, the VA RNA construct is not stably integrated into the genome of the cell.

In some embodiments, a cell comprises all three constructs (Rep/Cap construct, inducible helper construct, and the payload construct). In some embodiments, the three constructs are stably integrated into the genome of the cell. In some embodiments, the three constructs are not stably integrated into the genome of the cell. In some embodiments, the three constructs are separately stably integrated into the genome of the cell. In some embodiments, the three constructs are not separately stably integrated into the genome of the cell. In some embodiments, a plurality of the three constructs are stably integrated into the genome of the cell. In some embodiments, a plurality of the three constructs are not stably integrated into the genome of the cell. In some embodiments, a plurality of the three constructs are separately stably integrated into the genome of the cell. In some embodiments, a plurality of the three constructs are not separately stably integrated into the genome of the cell. In some embodiments, the cell, the inducible helper construct comprises a VA RNA construct as described herein. In some embodiments, cell further comprises the VA RNA construct.

In some embodiments, a VA RNA construct is a polynucleotide construct coding for a VA RNA, wherein a sequence coding for the VA RNA comprises at least two mutations in an internal promoter. In some embodiments, the sequence coding for the VA RNA comprises a sequence coding for a transcriptionally dead VA RNA. In some embodiments, the sequence coding for the VA RNA comprises a deletion of from about 5-10 nucleotides in the promoter region. In some embodiments, the sequence coding for the VA RNA comprises at least one mutation. In some embodiments, the at least one mutation is in the A Box promoter region. In some embodiments, the at least one mutation is in the B Box promoter region. In some embodiments, the at least one mutation is G16A and G60A. In some embodiments, the expression of the VA RNA is under the control of an RNA polymerase III promoter. In some embodiments, the expression of the VA RNA is under the control of an interrupted RNA polymerase III promoter. In some embodiments, the expression of the VA RNA is under the control of a U6 or U7 promoter. In some embodiments, the expression of the VA RNA is under the control of an interrupted U6 or U7 promoter. In some embodiments, the polynucleotide construct comprises upstream of the VA RNA gene sequence, from 5′ to 3′: a) a first part of a U6 or U7 promoter sequence; b) a first recombination site; c) a stuffer sequence; d) a second recombination site; e) a second part of a U6 or U7 promoter sequence. In some embodiments, the stuffer sequence is excisable by a recombinase. In some embodiments, the stuffer sequence comprises a sequence encoding a gene. In some embodiments, the stuffer sequence comprises a promoter. In some embodiments, the promoter is a constitutive promoter. In some embodiments, the promoter is a CMV promoter. In some embodiments, the gene encodes a detectable marker or a selectable marker. In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the stuffer sequence further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the stuffer sequence further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109. In some embodiments, the detectable marker comprises a luminescent marker or a fluorescent marker. In some embodiments, the fluorescent marker is GFP, EGFP, RFP, CFP, BFP, YFP, or mCherry. In some embodiments, the VA RNA construct further comprises a sequence coding for a recombinase. In some embodiments, the recombinase is exogenously provided. In some embodiments, the recombinase is a site-specific recombinase. In some embodiments, the recombinase is a Cre polypeptide or a Flippase polypeptide. In some embodiments, the Cre polypeptide is fused to a ligand binding domain. In some embodiments, the ligand binding domain is a hormone receptor. In some embodiments, the hormone receptor is an estrogen receptor. In some embodiments, the estrogen receptor comprises a point mutation. In some embodiments, the estrogen receptor is ERT2. In some embodiments, the recombinase is a Cre-ERT2 polypeptide. In some embodiments, the first recombination site is a first lox sequence and the second recombination site is a second lox sequence. In some embodiments, the first lox sequence is a first loxP site and the second lox sequence is a second loxP site. In some embodiments, the first recombination site is a first FRT site and the second recombination site is a second FRT site. The polynucleotide construct of any one of any embodiment disclosed herein, further comprising a sequence coding for a selectable marker.

In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the polynucleotide construct further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the polynucleotide construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109. In some embodiments, the VA RNA construct comprises a sequence having at least 70%, 80%, 90%, 95%, 99%, or 100% sequence identity to any one of SEQ ID NO: 13-SEQ ID NO: 19 or SEQ ID 23-SEQ ID NO: 26. In some embodiments, VA RNA construct has at least 70%, 80%, 90%, 95%, 99%, or 100% sequence identity to any one of SEQ ID NO: 13-SEQ ID NO: 19 or SEQ ID 23-SEQ ID NO: 26.

In some embodiments, the cell is a mammalian cell or insect cell. In some embodiments, the cell is a HEK293 cell, HeLa cell, CHO cell, or SF9 cell. In some embodiments, the cell expresses E1A protein and E1B protein. In some embodiments, the cell further comprises a payload construct. In some embodiments, the payload construct comprises a sequence having at least 70%, 80%, 90%, 95%, 99%, or 100% sequence identity to SEQ ID NO: 33. In some embodiments, the payload construct comprises a sequence of a payload flanked by ITR sequences. In some embodiments, expression of the sequence of the payload is driven by a constitutive promoter. In some embodiments, the constitutive promoter and sequence of the payload are flanked by ITR sequences. In some embodiments, the sequence of the payload comprises a polynucleotide sequence coding for a gene. In some embodiments, the gene codes for a selectable marker or detectable marker. In some embodiments, the gene codes for a therapeutic polypeptide or transgene. In some embodiments, the sequence of the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the payload construct is stably integrated into the genome of the cell. In some embodiments, a plurality of the payload construct are stably integrated into the genome of the cell. In some embodiments, the plurality of the payload constructs are separately stably integrated into the genome of the cell. In some embodiments, the payload construct further comprises a sequence coding for a selectable marker or detectable marker outside of the ITR sequences. In some embodiments, the selectable marker is a mammalian cell selection element.

In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the payload construct further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the payload construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, the selectable marker is outside of the ITR sequences on the payload construct. In some embodiments, the selectable marker outside of the ITR sequences is a split intein linked to an N-terminus of the auxotrophic protein or split intein linked to a C-terminus of the auxotrophic protein. In some embodiments, the selectable marker outside of the ITR sequences is a leucine zipper linked to an N-terminus of the auxotrophic or leucine zipper linked to a C-terminus of the auxotrophic. In some embodiments, the selectable marker outside of the ITR sequences is a split intein linked to an N-terminus of the antibiotic resistance protein or split intein linked to a C-terminus of the antibiotic resistance protein. In some embodiments, the selectable marker outside of the ITR sequences is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the payload construct is in a plasmid. In some embodiments, the payload construct is in a bacterial artificial chromosome or yeast artificial chromosome. In some embodiments, the payload construct is stably integrated into the genome of the cell. In some embodiments, the payload construct is a synthetic nucleic acid construct. In some embodiments, the cell is capable of producing an rAAV virion that encapsidates the sequence of the payload. In some embodiments, the cell is capable of producing an rAAV virion upon addition of at least one triggering agent.

In some embodiments, the cell is selected to comprise a high copy number of a construct integrated into the cell. The construct can be the helper construct, the Rep/Cap construct, the payload construct, or any combination thereof. In some embodiments, the construct comprises an attenuated promoter that drives expression the selectable marker results in selection of a cell having integrated a high copy number of the construct into the cell genome. An attenuated promoter can be a mutated EF1alpha promoter, such as an attenuated EF1alpha promoter comprising SEQ ID NO: 132. In some embodiments, the construct comprises a selectable marker having weak activity, such as a selectable marker mutated to have decreased enzymatic activity, results in selection of a cell having integrated a high copy number of the construct into the cell genome. For example, the selectable marker can be a mutated GS, having a mutation at R324C, R324S, or R341C mutation as compared to SEQ ID NO: 112. In some embodiments, culturing the cell comprising a construct having a selectable marker with an inhibitor of the selectable marker results in selection of the cell having integrated a high copy number of the construct into the cell genome. For example, the selectable marker can be GS and the cell can be cultured with methionine sulfoximine (MSX). In some embodiments, the selectable maker is DHFR and the cell can be cultured with methotrexate, ochratoxin A, alpha-methyl-tyrosine, alpha-methyl-phenylalanine, beta-2-thienyl-DL-alanine, or fenclonine. The selectable marker can be any auxotrophic protein or any antibiotic resistance protein. The selectable marker can any auxtrophic selection element as described herein. The selectable marker can be any selectable marker as described herein. In some embodiments, the selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the construct further comprises a helper enzyme, such as GTP-CH1. In some embodiments, the helper enzyme comprises at least 80%, 85%, 90%, 95%, 98%, 99%, or 100% sequence identity to SEQ ID NO: 99. In some embodiments, the selectable marker and helper enzyme of the construct comprises at least 80%, 85%, 90%, 95%, 98%, 99%, or 100% sequence identity to SEQ ID NO: 101-SEQ ID NO: 109. In some embodiments, the selection occurs in media comprising, for example, an antibiotic, or lacking nutrient required for cell growth accordingly for the selectable marker being used. In some embodiments, the media is supplemented with a cofactor or a cofactor precursor accordingly for the selectable marker being used and/or the helper enzyme being used. In some embodiments, the cofactor or cofactor precursor is tetrahydrobiopterin (BH4) or 7,8-dihydrobiopterin (7,8-BH2).

In some embodiments, this cell is capable of producing an rAAV virion upon addition of at least one triggering agent. In some embodiments, the rAAV virion comprising the capsid protein and the payload nucleic acid sequence have an infectivity of no less than 50%, 60%, 70%, 80%, 90%, 95%, or 99% at an MOI of 1×105 vg/target cell or less. In some embodiments, the rAAV virions have an increased infectivity compared rAAV virions produced by an otherwise comparable the population of cells capable of producing rAAV virions upon transient transfection at the same MOI. In some embodiments, the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI. In some embodiments, the rAAV virions have at least 50%, 60%, 70%, 80%, 90%, 95%, 97%, 98%, 99%, or 100% infectivity as compared to AAV virions produced by a cell having wildtype AAV at the same MOI. In some embodiments, the rAAV virions have at least 50%, 60%, 70%, 80%, 90%, 95%, 97%, 98%, 99%, or 100% infectivity as compared to AAV virions at the same MOI. In some embodiments, the AAV virions are wildtype AAV virions produced by a cell having wildtype AAV. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the cell is conditionally capable of producing rAAV virions having a F:E ratio of no less than 0.5, 0.6, 0.7, 0.8, 0.9, 0.95, 0.97, or 0.99. In some embodiments, the rAAV virions have a F:E ratio of no less than 0.5, 0.6, 0.7, 0.8, 0.9, 0.95, 0.97, or 0.99 prior to purification. In some embodiments, the cell is conditionally capable of producing rAAV virions having a encapsidation ratio of no less than 0.5, 0.6, 0.7, 0.8, 0.9, 0.95, 0.97, or 0.99. In some embodiments, the rAAV virions have a payload encapsidation ratio of no less than 0.5, 0.6, 0.7, 0.8, 0.9, 0.95, 0.97, or 0.99 prior to purification. In some embodiments, the rAAV virions have a concentration of greater than 1×10¹¹ or no less than 5×10¹¹, 1×10¹², 5×10¹², 1×10¹³ or 1×10¹⁴ viral genomes per milliliter prior to purification. In some embodiments, the cell is capable of producing rAAV virions comprising the payload nucleic acid sequence at a titer of greater than 1× 10¹¹ or no less than 5×10¹¹, 1×10¹², 5×10¹², 1×10¹³ or 1×10¹⁴ viral genomes per milliliter. In some embodiments, the cell is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification. In some embodiments, this cell is expanded to produce a population of cells. In some embodiments, the population of cells produces a stable cell line as described herein. In some embodiments, this cell is passaged at least three times. In some embodiments, this cell can be passaged up to 60 times. In some embodiments, this cell can be passage more than 60 times. In some embodiments, the cell maintains the ability to be conditionally induced after each passage.

### Population of Cells Comprising a Construct

In some embodiments, a population of cells comprise one construct (Rep/Cap construct, inducible helper construct, and the payload construct). In some embodiments, the one construct is stably integrated into the genomes of the cells. In some embodiments, the one construct is not stably integrated into the genomes of the cells. In some embodiments, a plurality of the one construct is stably integrated into the genomes of the cells. In some embodiments, a plurality of the one construct is not stably integrated into the genomes of the cells. In some embodiments, a population of cells comprises two constructs (any combination of Rep/Cap construct, inducible helper construct, and the payload construct). In some embodiments, the two constructs are stably integrated into the genomes of the cells. In some embodiments, the two constructs are not stably integrated into the genomes of the cells. In some embodiments, the two constructs are separately stably integrated into the genomes of the cells. In some embodiments, the two constructs are not separately stably integrated into the genomes of the cells. In some embodiments, a plurality of the two constructs are stably integrated into the genome of the cell. In some embodiments, a plurality of the two constructs are not stably integrated into the genome of the cell. In some embodiments, a plurality of the two constructs are separately stably integrated into the genomes of the cells. In some embodiments, a plurality of the two constructs are not separately stably integrated into the genomes of the cells. In some embodiments, a cell comprises the Rep/Cap construct and the inducible helper construct. In some embodiments, the cell, the inducible helper construct comprises a VA RNA construct as described herein. In some embodiments, cell further comprises the VA RNA construct as described herein. In some embodiments, the VA RNA construct is stably integrated into the genomes of the cells. In some embodiments, the VA RNA construct is not stably integrated into the genomes of the cells.

In some embodiments, a population of cells comprises all three constructs (Rep/Cap construct, inducible helper construct, and the payload construct). In some embodiments, the three constructs are stably integrated into the genomes of the cells. In some embodiments, the three constructs are not stably integrated into the genomes of the cells. In some embodiments, the three constructs are separately stably integrated into the genomes of the cells. In some embodiments, the three constructs are not separately stably integrated into the genomes of the cells. In some embodiments, a plurality of the three constructs are stably integrated into the genomes of the cells. In some embodiments, a plurality of the three constructs are not stably integrated into the genomes of the cells. In some embodiments, a plurality of the three constructs are separately stably integrated into the genomes of the cells. In some embodiments, a plurality of the three constructs are not separately stably integrated into the genomes of the cells. In some embodiments, the inducible helper construct comprises a VA RNA construct as described herein. In some embodiments, a population of cells further comprises the VA RNA construct separately integrated into the genomes of the cells. In some embodiments, a population of cells further comprises the VA RNA construct that are not separately integrated into the genomes of the cells.

In some embodiments, one or more constructs comprising the elements of the Rep/Cap construct, the helper construct, and/or the payload construct are stably integrated into the genomes of the cells. In some embodiments, one or more constructs comprising the elements of the Rep/Cap construct, the helper construct, and/or the payload construct are not stably integrated into the genomes of the cells. In some embodiments, one or more constructs comprising the elements of the Rep/Cap construct, the helper construct, and/or the payload construct are separately stably integrated into the genomes of the cells. In some embodiments, one or more constructs comprising the elements of the Rep/Cap construct, the helper construct, and/or the payload construct are not separately stably integrated into the genomes of the cells. In some embodiments, a plurality of the one or more constructs comprising the elements of the Rep/Cap construct, the helper construct, and/or the payload construct are separately stably integrated into the genomes of the cells. In some embodiments, the plurality of the one or more constructs comprising the elements of the Rep/Cap construct, the helper construct, and/or the payload construct are not separately stably integrated into the genomes of the cells. In some embodiments, cell further comprises the VA RNA construct as described herein. In some embodiments, the VA RNA construct is stably integrated into the genomes of the cells. In some embodiments, the VA RNA construct is not stably integrated into the genomes of the cells.

In some embodiments, the population of cells is selected to comprise a high copy number of a construct integrated into the population of cells. The construct can be the helper construct, the Rep/Cap construct, the payload construct, or any combination thereof. In some embodiments, the construct comprises an attenuated promoter that drives expression the selectable marker results in selection of the population of cells having integrated a high copy number of the construct into the cell genome. An attenuated promoter can be a mutated EF1alpha promoter, such as an attenuated EF1alpha promoter comprising SEQ ID NO: 132. In some embodiments, the construct comprises a selectable marker having weak activity, such as a selectable marker mutated to have decreased enzymatic activity, results in selection of the population of cells having integrated a high copy number of the construct into the cell genome. For example, the selectable marker can be a mutated GS, having a mutation at R324C, R324S, or R341C mutation as compared to SEQ ID NO: 112. In some embodiments, culturing the population of cells comprising the construct having a selectable marker, with an inhibitor of the selectable marker results in selection of the population of cells having integrated a high copy number of the construct into the cell genome of the population of cells. For example, the selectable marker can be GS and the cell can be cultured with methionine sulfoximine (MSX). In some embodiments, the selectable maker is DHFR and the cell can be cultured with methotrexate, ochratoxin A, alpha-methyl-tyrosine, alpha-methyl-phenylalanine, beta-2-thienyl-DL-alanine, or fenclonine. The selectable marker can be any auxotrophic protein or any antibiotic resistance protein. The selectable marker can any auxtrophic selection element as described herein. The selectable marker can be any selectable marker as described herein. In some embodiments, the selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the construct further comprises a helper enzyme. In some embodiments, the selectable marker and helper enzyme of the construct comprises at least 80%, 85%, 90%, 95%, 98%, 99%, or 100% sequence identity to SEQ ID NO: 101-SEQ ID NO: 109. In some embodiments, the selection occurs in media comprising, for example, an antibiotic, or lacking nutrient (e.g., a cofactor) required for cell growth accordingly for the selectable marker being used.

A population of cells as disclosed herein can be capable of producing rAAV virions having a encapsidation ratio of no less than 0.5, 0.6, 0.7, 0.8, 0.9, 0.95, 0.97, or 0.99. In some embodiments, the population of cells are a population of mammalian cells or a population of insect cells. In some embodiments, the population of cells are a population of HEK293 cells, HeLa cells, CHO cells, or SF9 cells. In some embodiments, the cell expresses E1A protein and E1B protein. In some embodiments, the population of cells further comprises a payload construct. In some embodiments, the payload construct comprises a sequence of a payload flanked by ITR sequences. In some embodiments, expression of the payload is driven by a constitutive promoter. In some embodiments, the constitutive promoter and sequence of the payload are flanked by ITR sequences. In some embodiments, the payload comprises a polynucleotide sequence encoding a gene. In some embodiments, the gene codes for a selectable marker or detectable marker. In some embodiments, the gene codes for a therapeutic polypeptide or transgene. In some embodiments, the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the payload construct is stably integrated into the genome of the cell. In some embodiments, the payload construct further comprises a sequence coding for a selectable marker or detectable marker outside of the ITR sequences.

In some embodiments, the selectable marker is a mammalian cell selection element. In some embodiments, the selectable marker is an auxotrophic selection element. In some embodiments, the auxotrophic selection element codes for an active protein. In some embodiments, the active protein is glutamine synthetase (GS), thymidylate synthase (TYMS), phenylalanine hydroxylase (PAH), or dihydrofolate reductase (DHFR). In some embodiments, PAH comprises at least 80%, 85%, 90%, 95%, 98%, 99%, or 100% sequence identity to SEQ ID NO: 90. In some embodiments, GS comprises at least 80%, 85%, 90%, 95%, 98%, 99%, or 100% sequence identity to SEQ ID NO: 112. In some embodiments, TYMS comprises at least 80%, 85%, 90%, 95%, 98%, 99%, or 100% sequence identity to SEQ ID NO: 123. In some embodiments, the auxotrophic selection element codes for an inactive protein that requires expression of a second auxotrophic selection element for activity. In some embodiments, the auxotrophic selection element codes for a C-terminal fragment of the auxotrophic protein Z-Cter and the second auxotrophic selection element codes for N-terminal fragment of an auxotrophic protein Z-Nter, or vice a versa. In some embodiments, the auxotrophic selection element codes for DHFR Z-Cter or DHFR Z-Nter. In some embodiments In some embodiments, the selectable marker is DHFR Z-Nter or DHFR Z-Cter. In some embodiments, the DHFR Z-Nter comprises a sequence having at least 70%, 80%, 90%, 95%, 99%, or 100% sequence identity to SEQ ID NO: 4. In some embodiments, the DHFR Z-Cter comprises a sequence having at least 70%, 80%, 90%, 95%, 99%, or 100% sequence identity to SEQ ID NO: 5. In some embodiments, the auxotrophic selection element codes for a C-terminal fragment of the auxotrophic protein fused to a C-terminal intein of a split intein and the second auxotrophic selection element codes for an N-terminal fragment of an auxotrophic protein fused to an N-terminal intein of a split intein. In some embodiments, the auxotrophic selection element codes for an N-terminal fragment of an auxotrophic protein fused to an N-terminal intein of a split intein and the second auxotrophic selection element codes for a C-terminal fragment of the auxotrophic protein fused to a C-terminal intein of a split intein. In some embodiments, the auxotrophic selection element codes for C-terminal fragment of PAH, GS, TYMS, or DHFR fused to a C-terminal intein of a split intein. In some embodiments, the auxotrophic selection element codes for an N-terminal fragment of PAH, GS, TYMS, or DHFR fused to a N-terminal intein of a split intein. In some embodiments, the selectable marker is an antibiotic resistance protein. In some embodiments, the selectable marker is a split intein linked to an N-terminus of the antibiotic resistance protein or split intein linked to a C-terminus of the antibiotic resistance protein. In some embodiments, the selectable marker is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the split intein is derived from the Nostoc punctiforme (Npu) DnaE intein, the Synechocystis species, strain PCC6803 (Ssp) DnaE intein, or the consensus DnaE intein (Cfa). In some embodiments, an N-terminal intein comprises at least 80%, 85%, 90%, 95%, 98%, 99%, or 100% sequence identity to SEQ ID NO: 140. In some embodiments, a C-terminal intein comprises at least 80%, 85%, 90%, 95%, 98%, 99%, or 100% sequence identity to SEQ ID NO: 141.

In some embodiments, the payload construct further comprises a sequence coding for a selectable marker and a helper enzyme, wherein expression of the helper enzyme facilitates growth of the cell in conjunction with the selectable marker. In certain embodiments, the helper enzyme is an enzyme that facilitates production of a molecule required for cell growth. For example, the helper enzyme may be required for production of a cofactor utilized by the functional enzyme to generate the molecule required for cell growth. In certain embodiments, the cell may produce the helper enzyme at low levels and the expression of the helper enzyme from the helper construct can increase helper enzyme levels thereby increasing production of the molecule required for cell growth, by, e.g., increasing levels of a co-factor required for enzyme activity. In some embodiments, the payload construct further encodes a helper enzyme involved in production of tyrosine from phenylalanine. In some embodiments, the helper enzyme facilitates PAH-mediated production of tyrosine from phenylalanine. In some embodiments, the helper enzyme catalyzes production a co-factor required by PAH for converting phenylalanine to tyrosine. In some embodiments, the helper enzyme is GTP cyclohydrolase I (GTP-CH1). In some embodiments, the helper enzyme comprises at least 80%, 85%, 90%, 95%, 98%, 99%, or 100% sequence identity to SEQ ID NO: 99. In some embodiments, the GTP-CH1 produces the cofactor (6R)-5,6,7,8-tetrahydrobiopterin (BH4) that is required for conversion of phenylalanine to tyrosine. In some embodiments, expression of GTP-CH1 facilitates growth of the host cell in conjunction with functional PAH upon application of the single selective pressure.

In some embodiments, a selectable marker comprises at least 80%, 85%, 90%, 95%, 98%, 99%, or 100% sequence identity to SEQ ID NO: 90-SEQ ID NO: 98, SEQ ID NO: 112-SEQ ID NO: 131, SEQ ID NO: 137, or SEQ ID NO: 138. In some embodiments, the selectable marker and helper enzyme comprises at least 80%, 85%, 90%, 95%, 98%, 99%, or 100% sequence identity to SEQ ID NO: 101-SEQ ID NO: 109.

In some embodiments, the selectable marker is outside of the ITR sequences on the payload construct. In some embodiments, the selectable marker outside of the ITR sequences is a split intein linked to an N-terminus of the auxotrophic protein or split intein linked to a C-terminus of the auxotrophic protein. In some embodiments, the selectable marker outside of the ITR sequences is a leucine zipper linked to an N-terminus of the auxotrophic or leucine zipper linked to a C-terminus of the auxotrophic. In some embodiments, the selectable marker outside of the ITR sequences is a split intein linked to an N-terminus of the antibiotic resistance protein or split intein linked to a C-terminus of the antibiotic resistance protein. In some embodiments, the selectable marker outside of the ITR sequences is a leucine zipper linked to an N-terminus of the antibiotic resistance protein or leucine zipper linked to a C-terminus of the antibiotic resistance protein. In some embodiments, the antibiotic resistance protein is for puromycin resistance or blasticidin resistance. In some embodiments, the payload construct is in a plasmid. In some embodiments, the payload construct is in a bacterial artificial chromosome or yeast artificial chromosome. In some embodiments, the payload construct is stably integrated into the genomes of the population of cells. A population of cells produced by expanding a cell of any one of any embodiment disclosed herein. In some embodiments, expanding comprises passaging the cell at least three times. In some embodiments, a cell of the population of cells is capable of conditionally producing recombinant AAV (rAAV) virions upon addition of at least two triggering agents. In some embodiments, the cell is capable of conditionally producing rAAV virions upon addition of at least two triggering agents. In some embodiments, the at least two triggering agents comprise doxycycline and tamoxifen. In some embodiments, the at least two triggering agents induce the expression and translocation of an excising element to the nucleus. In some embodiments, a cell of the population of cells is capable of conditionally producing rAAV virions upon addition of an excising element. In some embodiments, the excising element is a recombinase. In some embodiments, the excising element is a site-specific recombinase. In some embodiments, the excising element is a Cre polypeptide or a flippase polypeptide. In some embodiments, the excising element is hormone regulated. In some embodiments, the population of cells are conditionally capable of producing rAAV virions within which are packaged an expressible polynucleotide encoding a payload; and wherein a population of virions produced by the population of cells are more homogenous than a population of virions produced by an otherwise comparable the population of cells capable of producing rAAV virions upon transient transfection. In some embodiments, the population of virions produced by the population of cells has a ratio of viral genomes to transduction units of about 500:1 to 1:1. In some embodiments, the population of virions produced by the population of cells has a ratio of vector genomes to infectious unit of 100:1. In some embodiments, production of virions is inducible upon addition of a triggering agent. In some embodiments, production of virions is inducible upon addition of at least two triggering agents. In some embodiments, the population of cells is conditionally capable of producing rAAV virions having a payload encapsidation ratio of no less than 0.5, 0.6, 0.7, 0.8, 0.9, 0.95, 0.97, or 0.99. In some embodiments, the rAAV virions have a payload encapsidation ratio of no less than 0.5, 0.6, 0.7, 0.8, 0.9, 0.95, 0.97, or 0.99 prior to purification. In some embodiments, the population of cells are capable of reaching a viable cell density of no less than 1×10⁶, 2×10⁶, 5×10⁶, or 1×10⁷ cells per milliliter. In some embodiments, the rAAV virions have a concentration of greater than 1×10¹¹ or no less than 5×10¹¹, 1×10¹², 5×10¹², 1×10¹³ or 1×10¹⁴ viral genomes per milliliter prior to purification. In some embodiments, the population of cells is capable of producing rAAV virions comprising the payload nucleic acid sequence at a titer of greater than 1×10¹¹ or no less than 5×10¹¹, 1×10¹², 5×10¹², 1×10¹³ or 1× 10¹⁴ viral genomes per milliliter. In some embodiments, the population of cells is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification. In some embodiments, the rAAV virions comprising the capsid protein and the payload nucleic acid sequence have an infectivity of no less than 50%, 60%, 70%, 80%, 90%, 95%, or 99% at an MOI of 1×10⁵ vg/target cell or less. In some embodiments, the rAAV virions have an increased infectivity compared rAAV virions produced by an otherwise comparable the population of cells capable of producing rAAV virions upon transient transfection at the same MOI. In some embodiments, the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI. In some embodiments, the rAAV virions have at least 50%, 60%, 70%, 80%, 90%, 95%, 97%, 98%, 99%, or 100% infectivity as compared AAV virions AAV at the same MOI. In some embodiments, the AAV virions are wildtype AAV virions produced by a cell having wildtype AAV. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the cell is conditionally capable of producing rAAV virions having a F:E ratio of no less than 0.5, 0.6, 0.7, 0.8, 0.9, 0.95, 0.97, or 0.99. In some embodiments, the rAAV virions have a F:E ratio of no less than 0.5, 0.6, 0.7, 0.8, 0.9, 0.95, 0.97, or 0.99 prior to purification. In some embodiments, the cells are cryopreserved. In some embodiments, the cells are comprised within a vial, flask, syringe, or other suitable cell-storage container. In some embodiments, production of rAAV virions is inducible in the absence of a plasmid. In some embodiments, expression of AAV Rep and Cap proteins is inducible in the absence of a plasmid. In some embodiments, expression of the at least one or more helper proteins is inducible in the absence of a plasmid. In some embodiments, production of rAAV virions is inducible in the absence of a transfection agent. In some embodiments, expression of AAV Rep and Cap proteins is inducible in the absence of a transfection agent. In some embodiments, expression of the at least one or more helper proteins is inducible in the absence of a transfection agent. A second population of cell produced by expanding the population of cells of any one of the preceding embodiments. The second population of cells, wherein expanding the population of cells comprises passaging the population of cells at least three times. In some embodiments expanding the population of cells comprises passaging the population of cells from 3 to 60 times. In some embodiments, expanding the population of cells comprises passaging the population of cells at least 4, 5, 6, 7, 8, 9, 10, 15, 20, 25, 30, 35, 40, 45, 50, 55, or 60 times.

In some embodiments, an rAAV virion produced by the methods described herein have increased infectivity compared to a comparable rAAV virion produced by transient transfection methods.

### Stable Cell Line

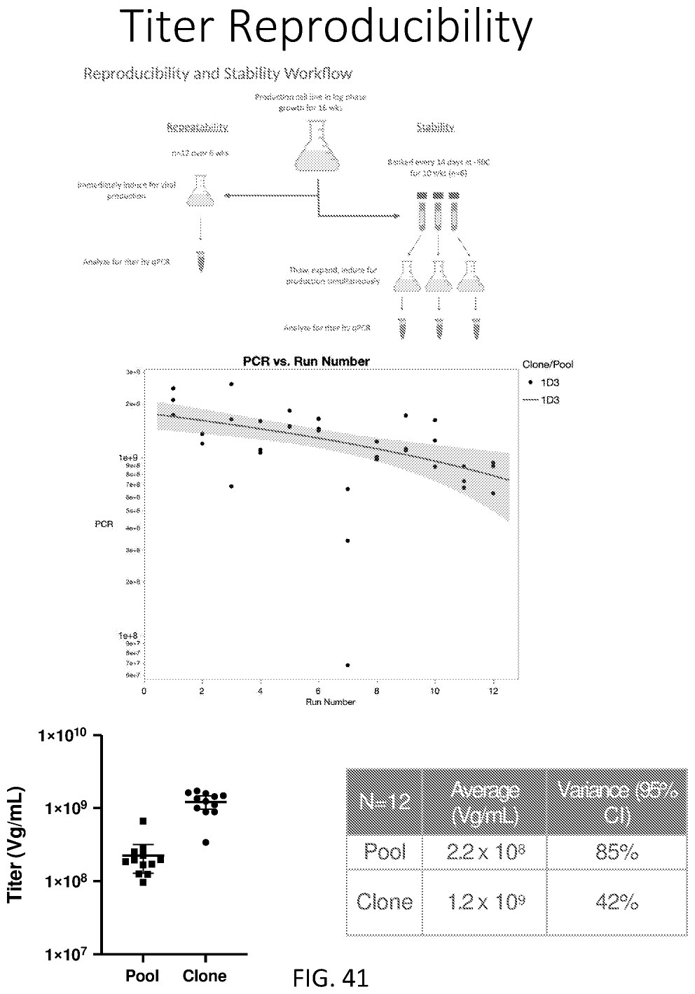

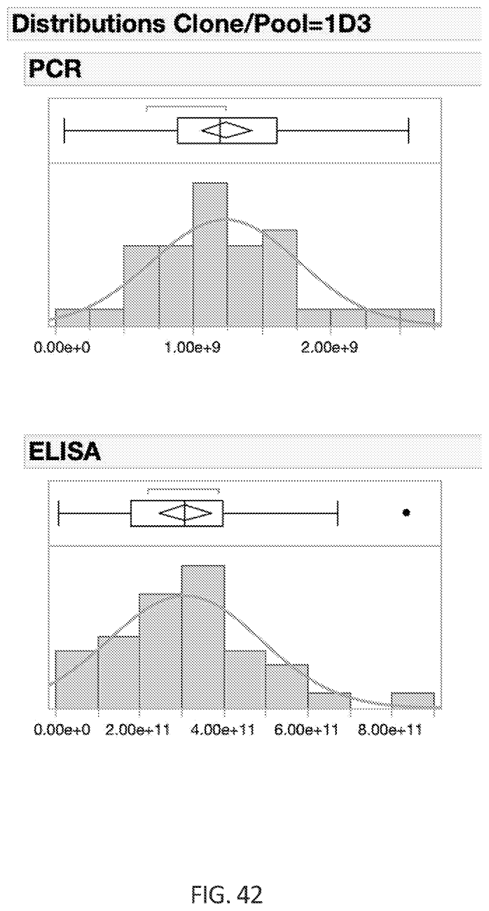

In some embodiments, a stable cell line is produced from the cell as described herein. In some embodiments, a stable cell line is produced from the population of cells as described herein. In some embodiments, the stable cell line is derived from a single cell and is monoclonal. The stable cell line can be a mammalian stable cell line. The stable cell line can be produced by expanding or passaging a cell as described herein. The stable cell line produced by expanding or passaging a single cell and is a monoclonal stable cell line can have high reproducibility of titer (see, e.g., FIG. 41 and FIG. 42). For example, induction of a first population of cells from the monoclonal stable cell line can produce titer that is no more than 50% different than titer produced from a second population of cells from the monoclonal stable cell line after induction. Induction of a first population of cells from the monoclonal stable cell line can produce titer that is no more than 40% different than titer produced from a second population of cells from the monoclonal stable cell line after induction. Induction of a first population of cells from the monoclonal stable cell line can produce titer that is no more than 35% different than titer produced from a second population of cells from the monoclonal stable cell line after induction. Induction of a first population of cells from the monoclonal stable cell line can produce titer that is no more than 30% different than titer produced from a second population of cells from the monoclonal stable cell line after induction. Induction of a first population of cells from the monoclonal stable cell line can produce titer that is no more than 25% different than titer produced from a second population of cells from the monoclonal stable cell line after induction. Induction of a first population of cells from the monoclonal stable cell line can produce titer that is no more than 20% different than titer produced from a second population of cells from the monoclonal stable cell line after induction. Induction of a first population of cells from the monoclonal stable cell line can produce titer that is no more than 10% different than titer produced from a second population of cells from the monoclonal stable cell line after induction. Induction of a first population of cells from the monoclonal stable cell line can produce titer that is approximately equal to titer produced from a second population of cells from the monoclonal stable cell line after induction. In some embodiments, the titer reproducibility occurs between at least a first population of cells from the monoclonal stable cell line and a second population of cells from the monoclonal stable cell line, but can occur between 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 different populations of cells. In some embodiments, the titer reproducibility occurs between 40, 50, 60, 70, 80, 90, or more different populations of cells. The different populations of cells from the monoclonal stable cell line can be cells taken and induced at different time points over a set time period, in which later time point populations of cells have undergone an increased number of passages compared to the early time point populations of cells. The set time period can be 1 week, 2 weeks, 3 weeks, 4 weeks, 6 weeks, 2 months, 4 months, 8 months, 10 months, 1 year, 2 years, 3 year, 4 year, 5 year, 6 years, 7 year, 8 years, 9 years, 10 years, any time period therebetween, or over 10 years. The monoclonal stable cell line can be any single cell stable cell line expanded to be a population of cells as described herein, e.g., a monoclonal stable cell line wherein the payload is progranulin. The monoclonal stable cell line having high reproducibility of titer can be a monoclonal stable cell line wherein the payload is progranulin.

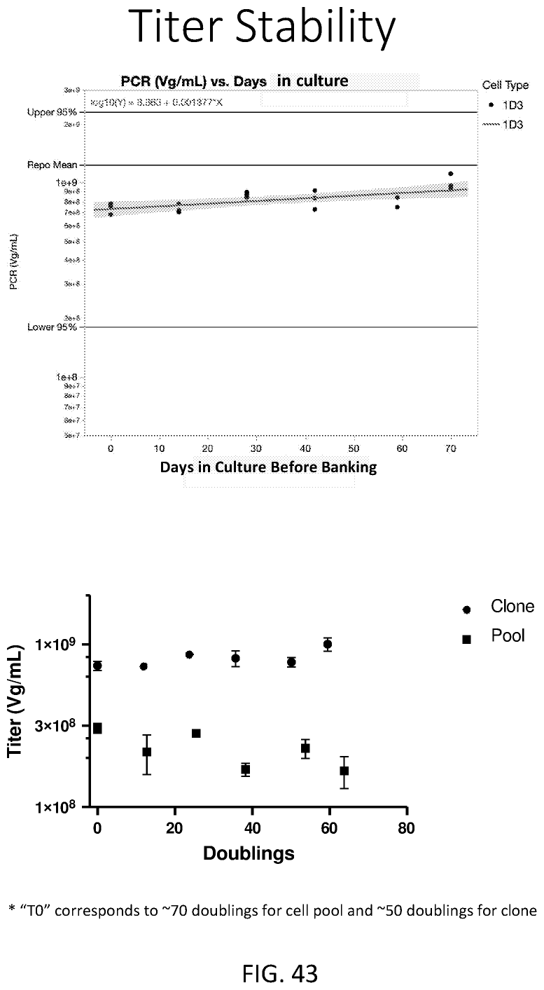

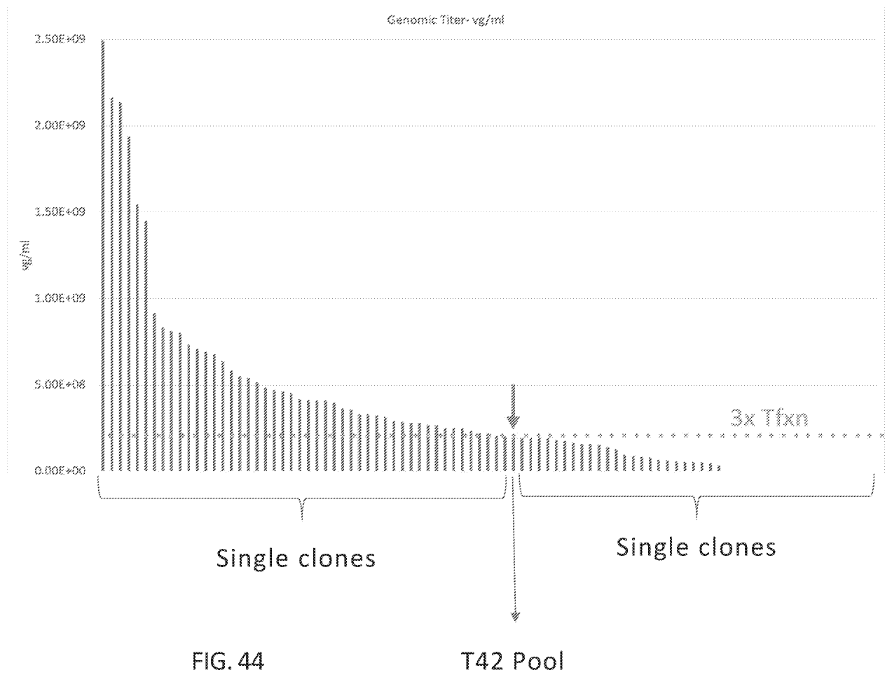

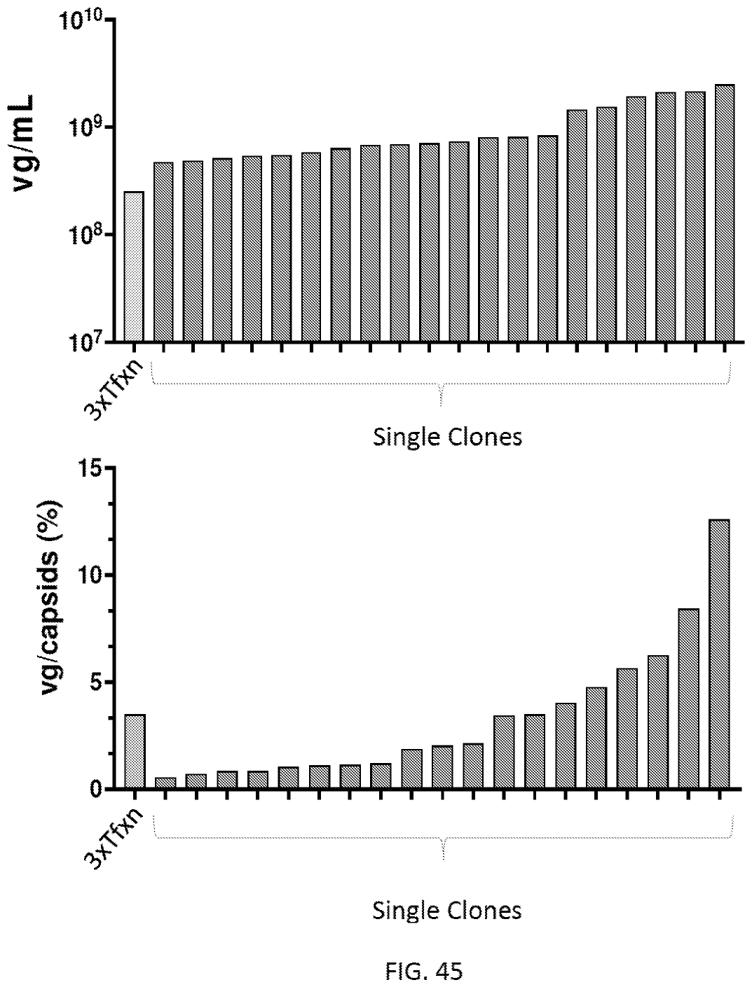

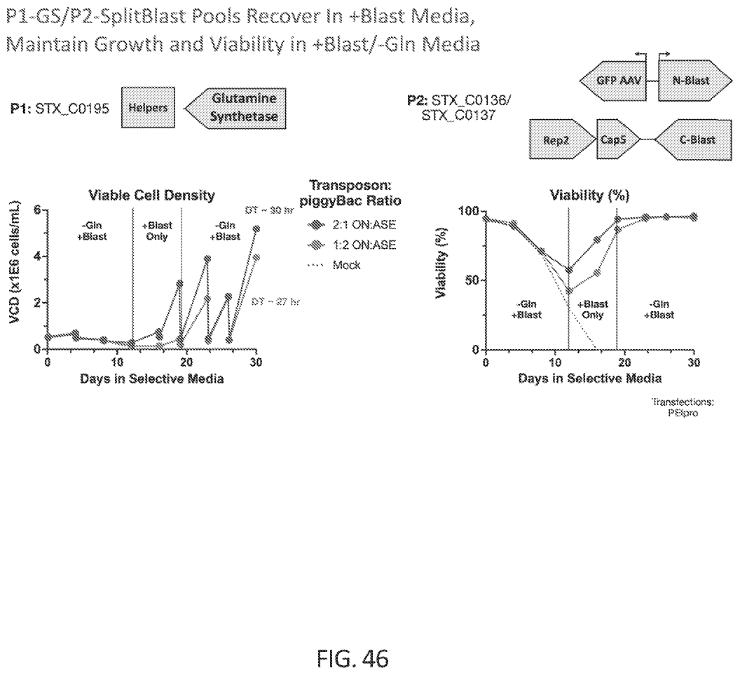

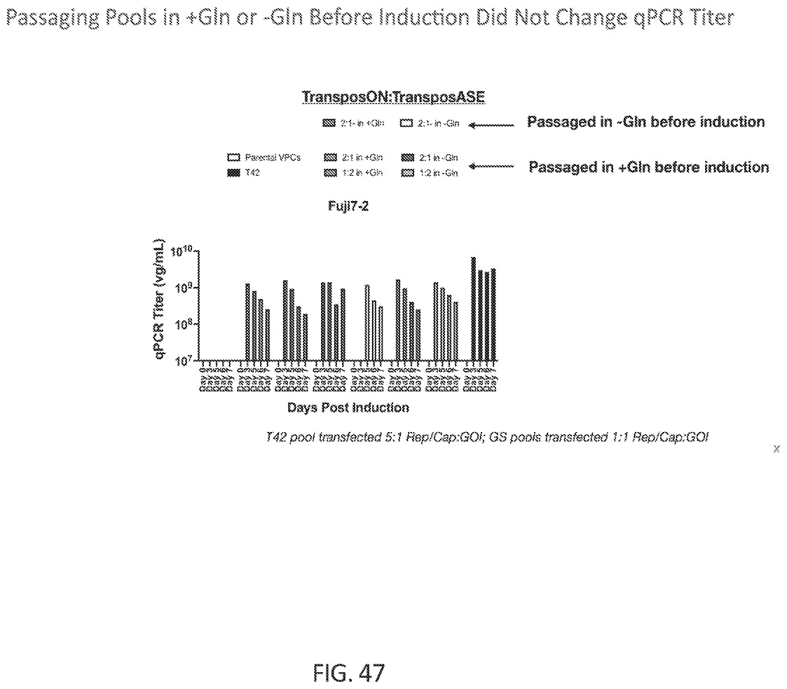

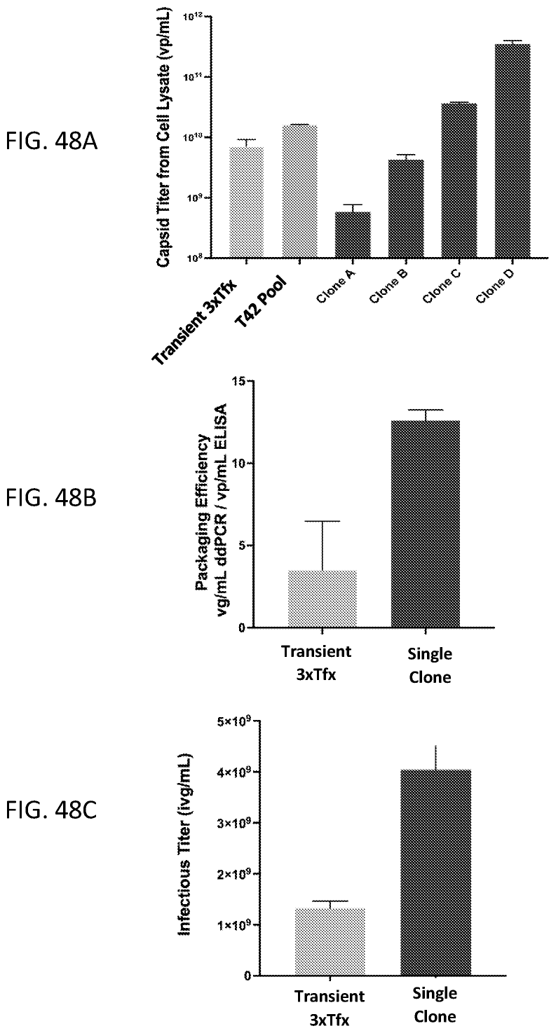

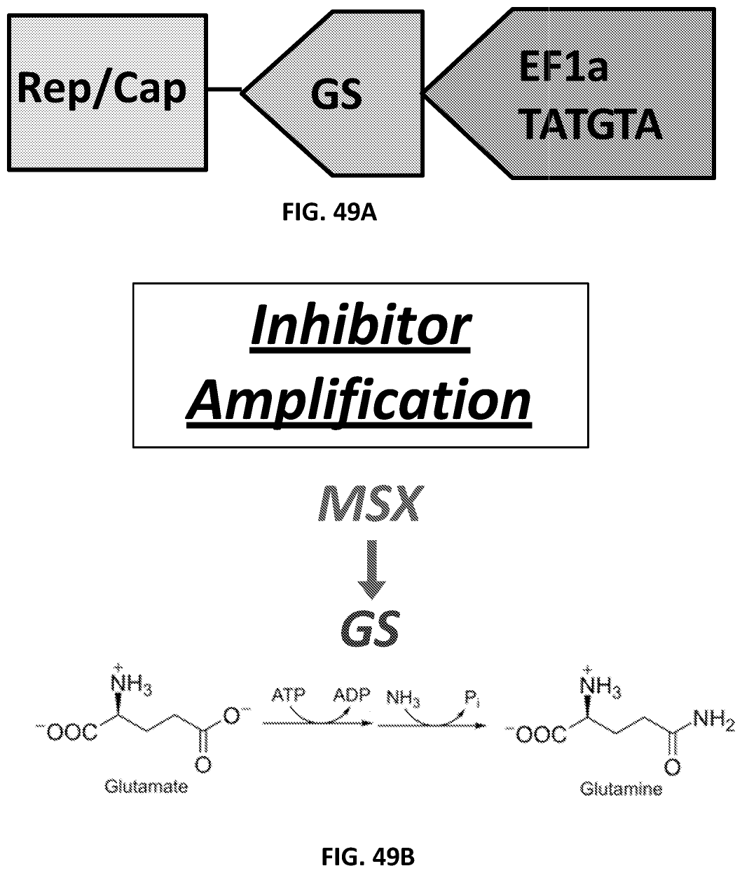

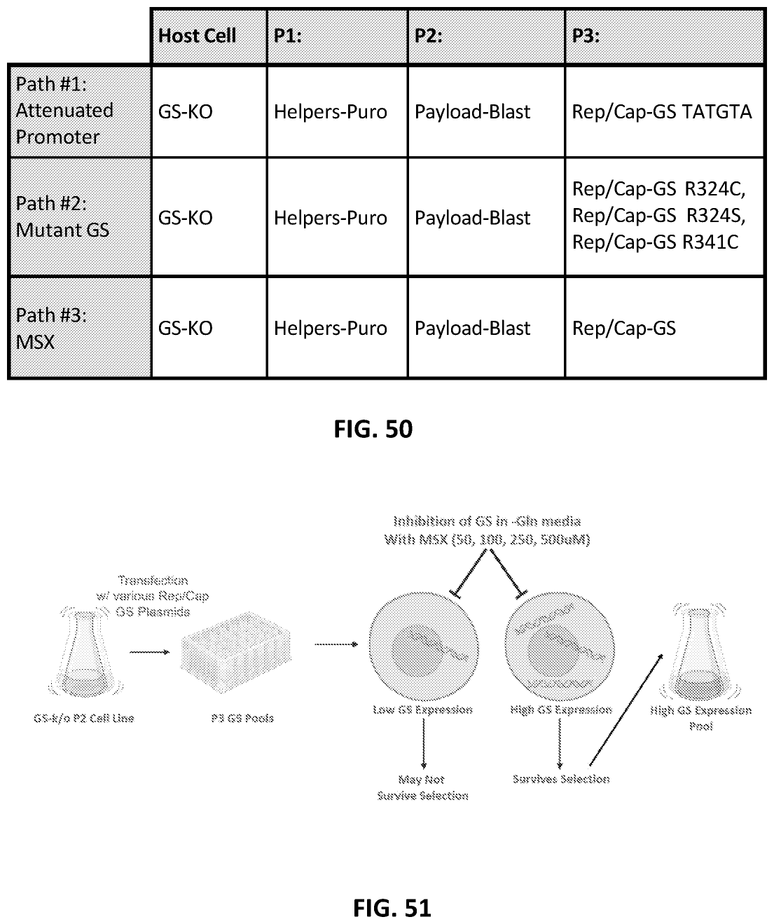

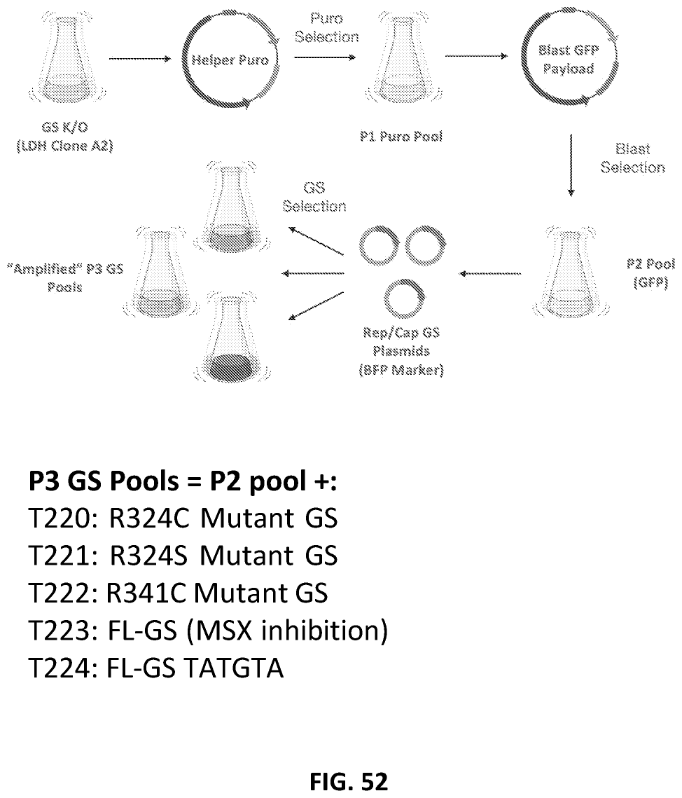

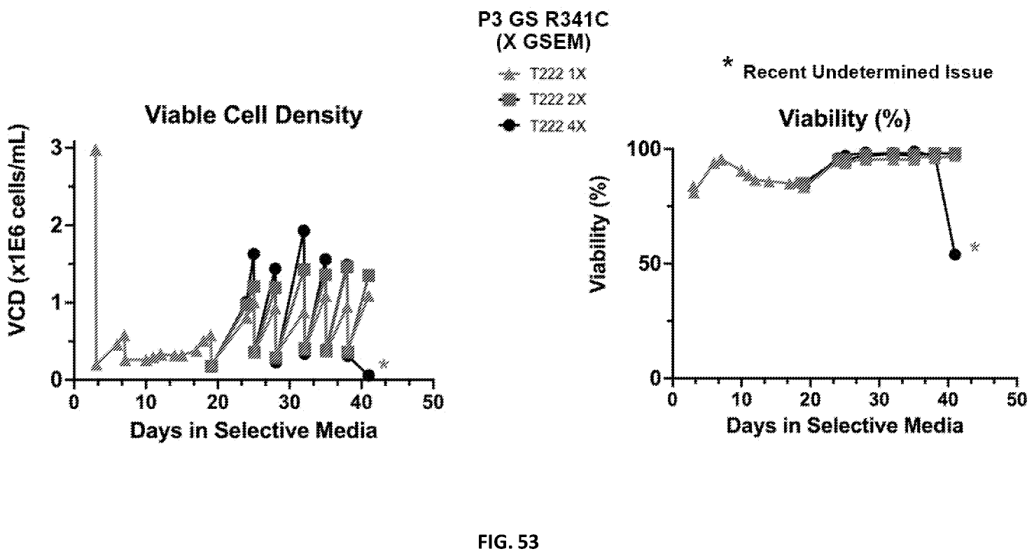

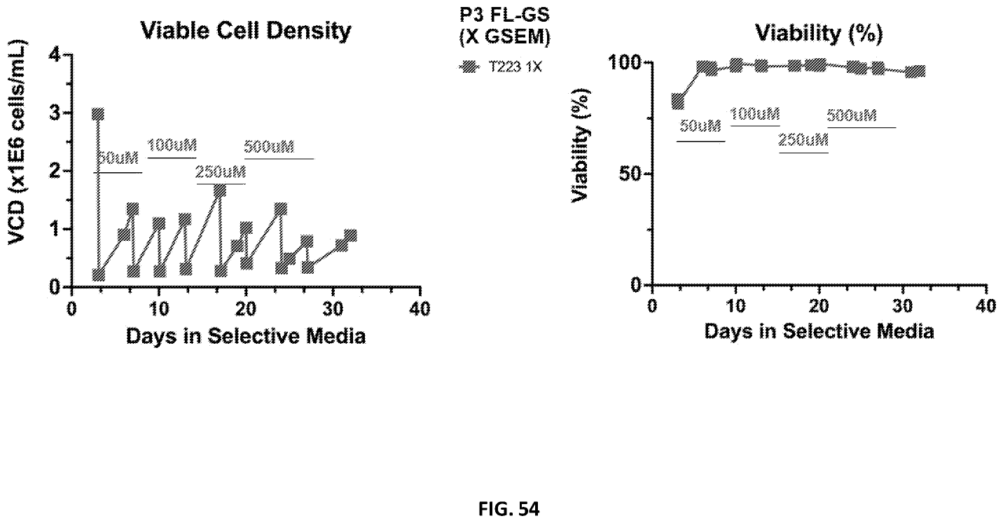

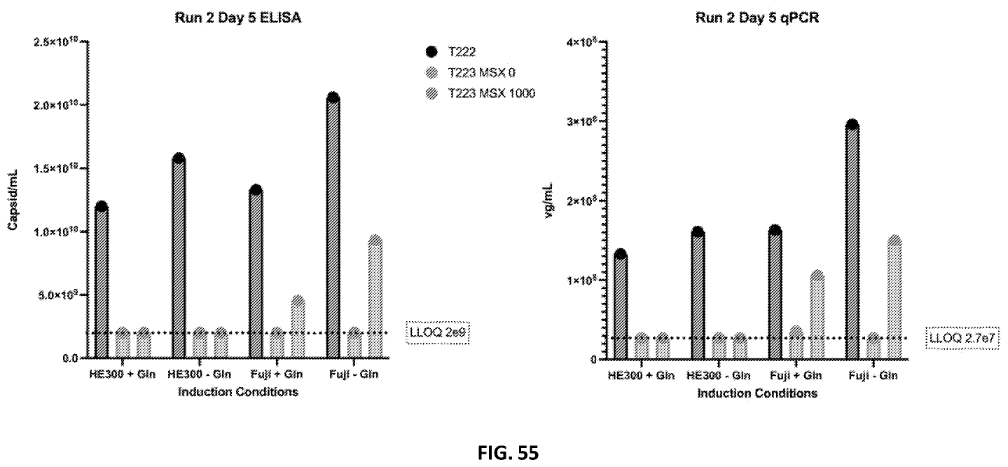

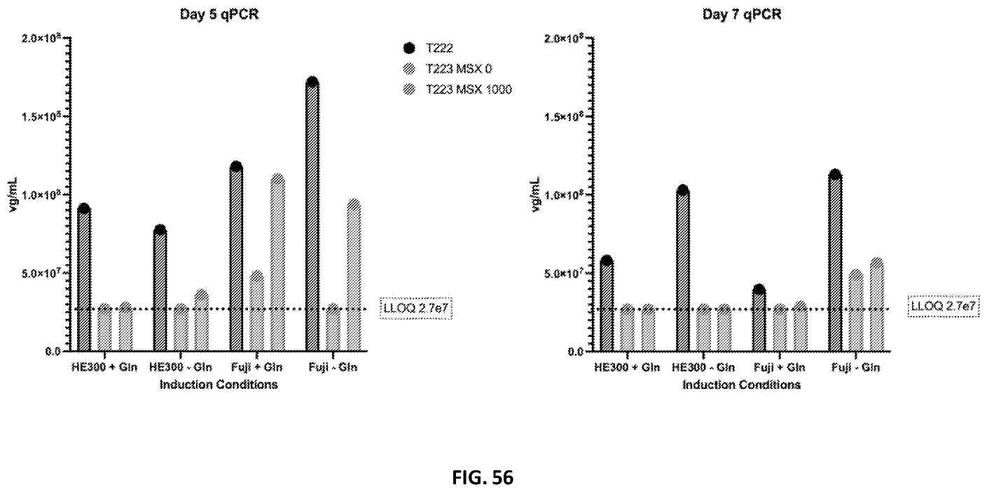

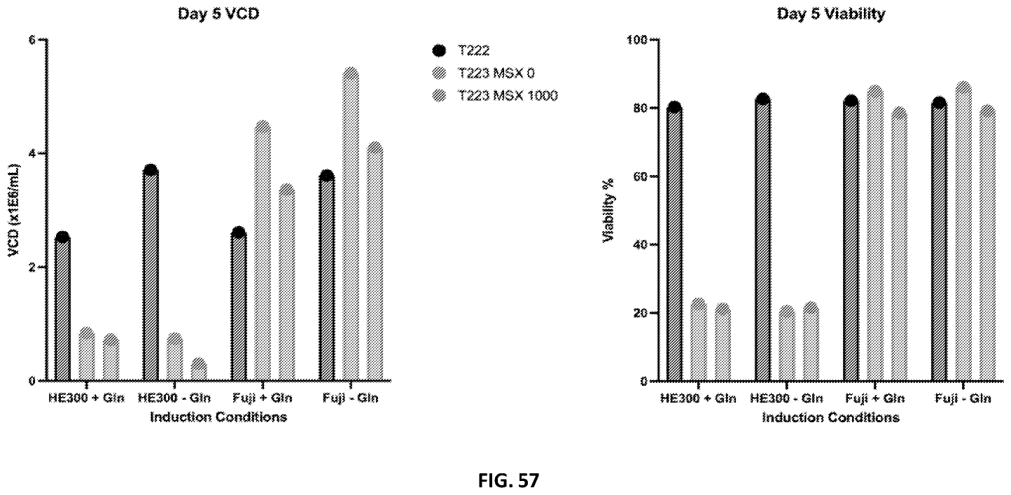

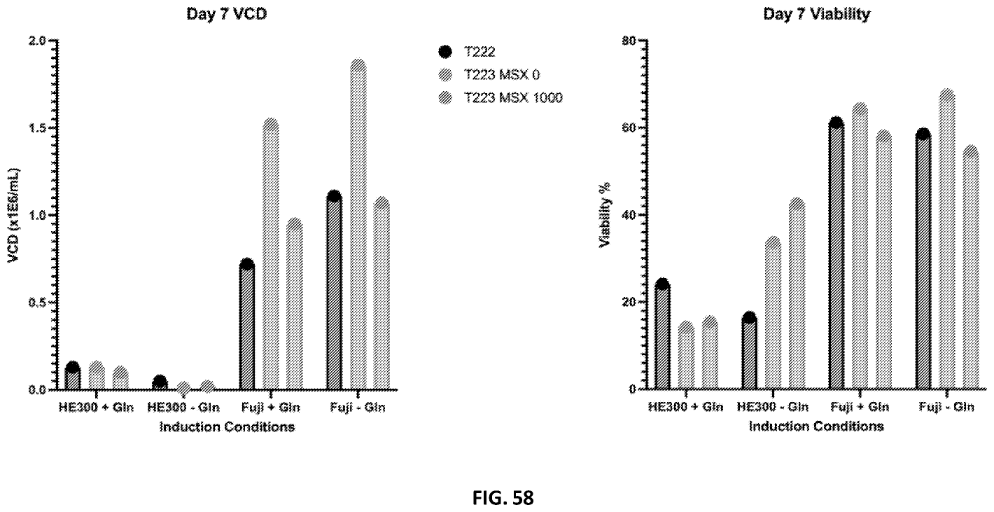

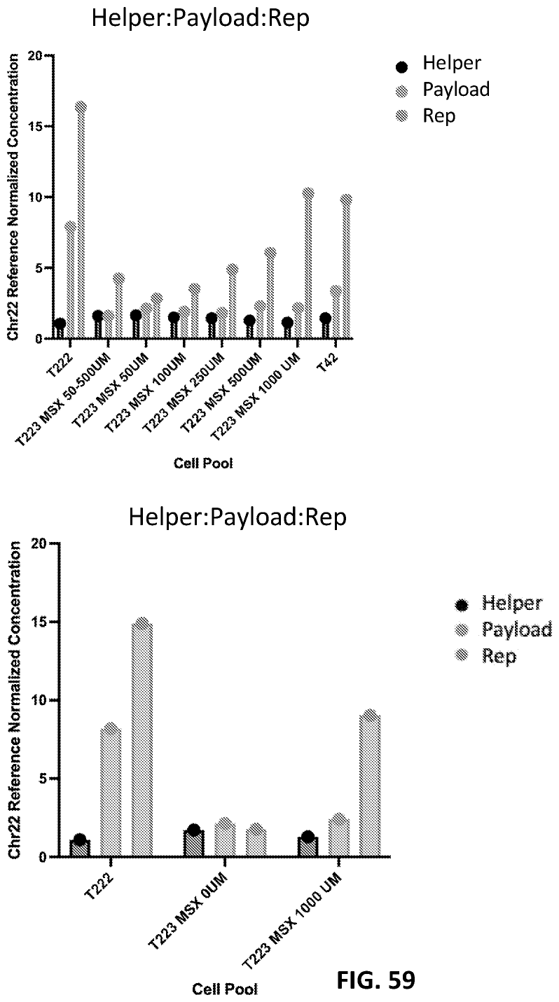

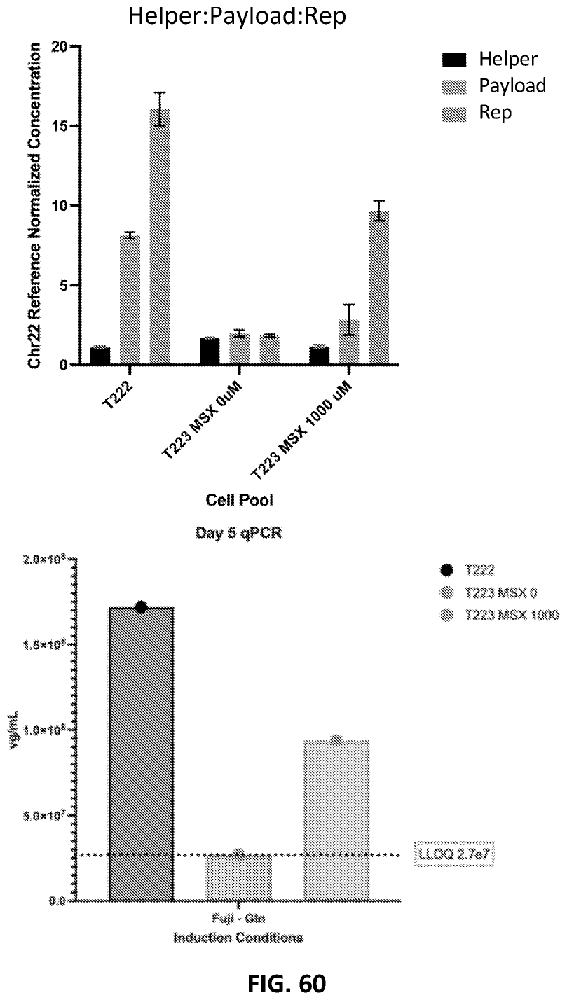

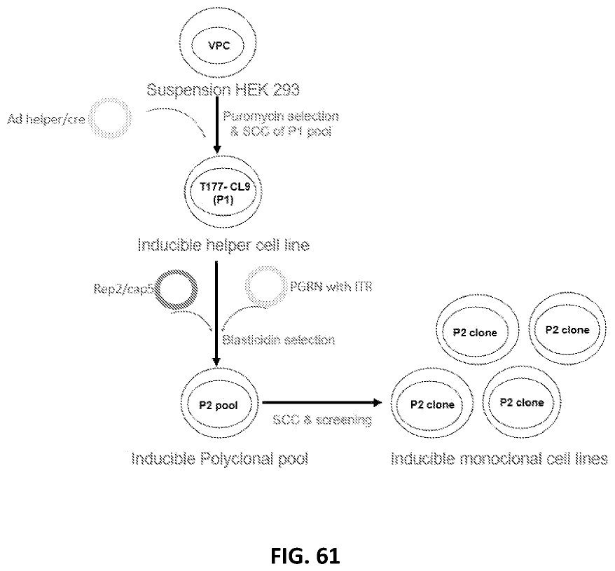

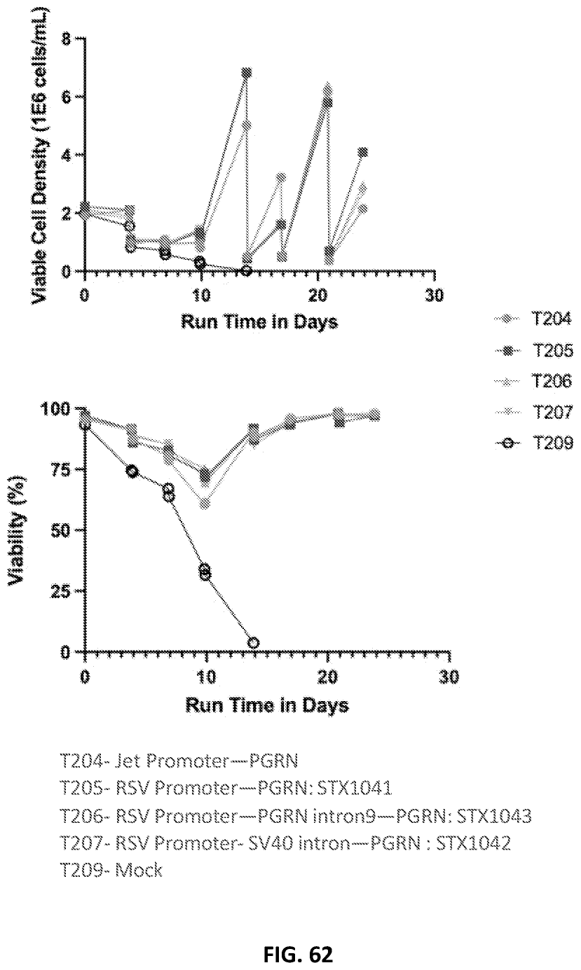

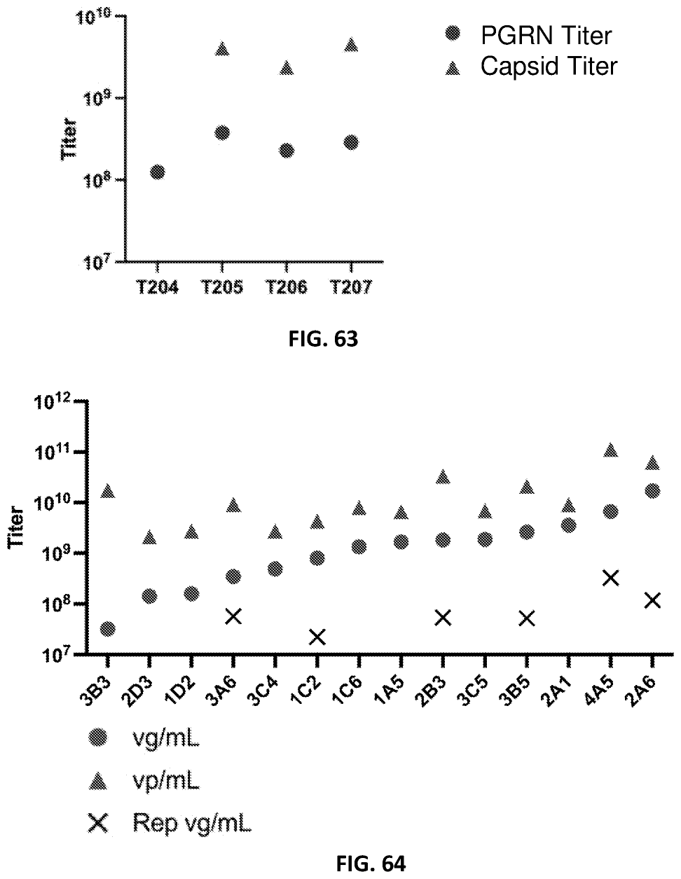

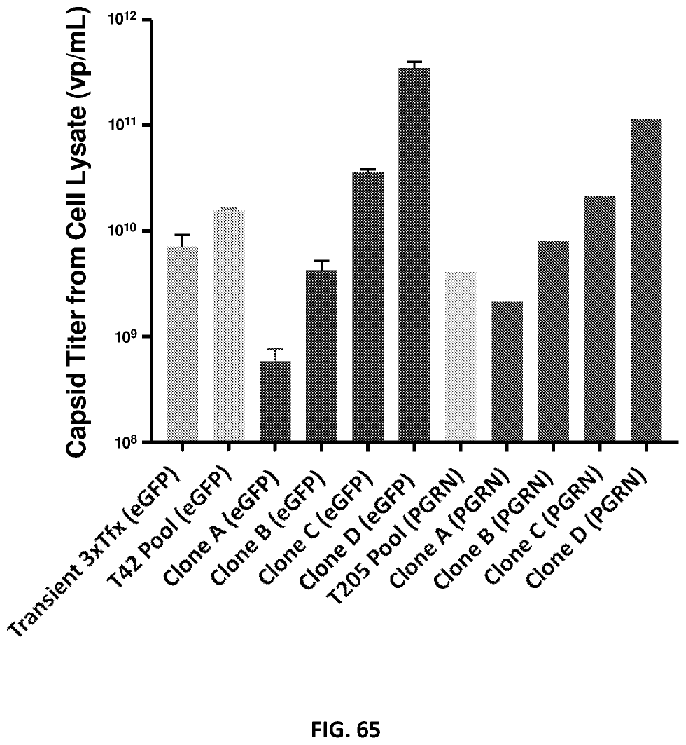

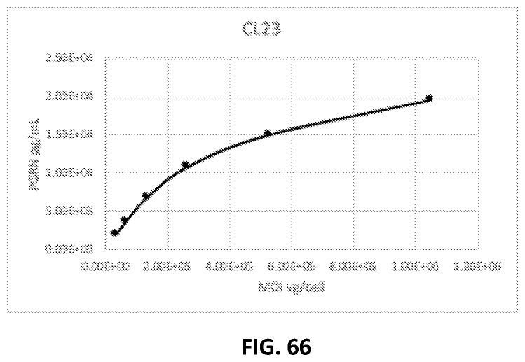

The stable cell line produced by expanding or passaging a single cell and is a monoclonal stable cell line can have high stability of titer (see, e.g., FIG. 43). For example, titer of banked cells is stable between the banked cells from different time points (having a different number of passages) but induced at the same time. For example, induction of a first population of cells (e.g., a first population of banked cells) from the monoclonal stable cell line can produce titer that is no more than 50% different than titer produced from a second population of cells (e.g., a second population of banked cells) from the monoclonal stable cell line after induction. Induction of a first population of cells (e.g., a first population of banked cells) from the monoclonal stable cell line can produce titer that is no more than 40% different than titer produced from a second population of cells (e.g., a second population of banked cells) from the monoclonal stable cell line after induction. Induction of a first population of cells (e.g., a first population of banked cells) from the monoclonal stable cell line can produce titer that is no more than 35% different than titer produced from a second population of cells (e.g., a second population of banked cells) from the monoclonal stable cell line after induction. Induction of a first population of cells (e.g., a first population of banked cells) from the monoclonal stable cell line can produce titer that is no more than 30% different than titer produced from a second population of cells (e.g., a second population of banked cells) from the monoclonal stable cell line after induction. Induction of a first population of cells (e.g., a first population of banked cells) from the monoclonal stable cell line can produce titer that is no more than 25% different than titer produced from a second population of cells (e.g., a second population of banked cells) from the monoclonal stable cell line after induction. Induction of a first population of cells (e.g., a first population of banked cells) from the monoclonal stable cell line can produce titer that is no more than 20% different than titer produced from a second population of cells (e.g., a second population of banked cells) from the monoclonal stable cell line after induction. Induction of a first population of cells (e.g., a first population of banked cells) from the monoclonal stable cell line can produce titer that is no more than 15% different than titer produced from a second population of cells (e.g., a second population of banked cells) from the monoclonal stable cell line after induction. Induction of a first population of cells (e.g., a first population of banked cells) from the monoclonal stable cell line can produce titer that is no more than 10% different than titer produced from a second population of cells (e.g., a second population of banked cells) from the monoclonal stable cell line after induction. Induction of a first population of cells (e.g., a first population of banked cells) from the monoclonal stable cell line can produce titer that is no more than 5% different than titer produced from a second population of cells (e.g., a second population of banked cells) from the monoclonal stable cell line after induction. Induction of a first population of cells (e.g., a first population of banked cells) from the monoclonal stable cell line can produce titer that is approximately equal to titer produced from a second population of cells (e.g., a second population of banked cells) from the monoclonal stable cell line after induction. In some embodiments, the titer stability occurs between at least a first population of cells (e.g., a first population of banked cells) from the monoclonal stable cell line and a second population of cells (e.g., a second population of banked cells) from the monoclonal stable cell line, but can occur between 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49, 50, 51, 52, 53, 54, 55, 56, 57, 58, 59, 60, 61, 62, 63, 64, or 65 different populations of cells. In some embodiments, the titer stability occurs between 70, 80, 90, or more different populations of cells. In some embodiments, the different populations of cells taken at different time points are at least 1 day, 2 day, 3 days, 1 week, 2 weeks, 3 weeks, 4 weeks, 5 weeks, 6 weeks, 7 weeks, 8 weeks, 9 weeks, 10 weeks, 11 weeks, 12 weeks, 4 months, 5 months, 6 months, any time point therebetween, or more apart. In some embodiments, the different populations of cells have undergone different a different number of passages, e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 41, 42, 43, 44, 45, 46, 47, 48, 49, 50, 51, 52, 53, 54, 55, 56, 57, 58, 59, 60, 61, 62, 63, 64, or 65 passages. The monoclonal stable cell line can be any single cell stable cell line expanded to be a population of cells as described herein, e.g., a monoclonal stable cell line wherein the payload is progranulin. The monoclonal stable cell line having high stability of titer can be a monoclonal stable cell line wherein the payload is progranulin.

In some embodiments, a stable cell line comprises the population of cells as disclosed herein. In some embodiments, the population of cells are derived from a single cell. In some embodiments, at least 70%, 80%, 90%, 95%, 99%, or 100% of the cells of the stable cell line are the population of cells as disclosed herein. A stable cell line derived from a cell as disclosed herein. A stable cell line expanded from a cell as disclosed herein. In some embodiments, the stable cell line is a mammalian stable cell line. In some embodiments, expression of one or more helper proteins is inducible in the absence of a plasmid. In some embodiments, expression of one or more helper proteins is inducible in the absence of a transfection agent. In some embodiments, expression of AAV Rep and Cap proteins is inducible in the absence of a plasmid. In some embodiments, expression of AAV Rep and Cap proteins is inducible in the absence of a transfection agent. In some embodiments, production of rAAV virions is inducible in the absence of a plasmid. In some embodiments, production of rAAV virions is inducible in the absence of a transfection agent. In some embodiments, the stable cell line is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter. In some embodiments, the stable cell line is capable of producing rAAV virions comprising the payload nucleic acid sequence at a concentration of greater than 1×10¹¹ or no less than 5×10¹¹, 1×10¹², 5×10¹², 1×10¹³ or 1×10¹⁴ viral genomes per milliliter prior to purification. In some embodiments, the stable cell line is conditionally capable of producing rAAV virions having a encapsidation ratio of no less than 0.5, 0.6, 0.7, 0.8, 0.9, 0.95, 0.97, or 0.99. In some embodiments, the rAAV virions have a encapsidation ratio of no less than 0.5, 0.6, 0.7, 0.8, 0.9, 0.95, 0.97, or 0.99 prior to purification. In some embodiments, the rAAV virions comprising the capsid protein and the payload nucleic acid sequence have an infectivity of no less than 50%, 60%, 70%, 80%, 90%, 95%, or 99%, at an MOI of 1×10⁵ vg/target cell or less. In some embodiments, the rAAV virions have an increased infectivity compared rAAV virions produced by an otherwise comparable the population of cells capable of producing rAAV virions upon transient transfection at the same MOI. In some embodiments, the rAAV virions have at least 1%, 5%, 10%, 15%, 20%, 30%, 40%, or 50% greater infectivity compared rAAV virions produced by an otherwise comparable the population of cells capable of producing rAAV virions upon transient transfection at the same MOI. In some embodiments, the rAAV virions have at least 50%, 60%, 70%, 80%, 90%, 95%, 97%, 98%, 99%, or 100% infectivity as compared to AAV virions produced by a cell having wildtype AAV at the same MOI. In some embodiments, the rAAV virions have at least 50%, 60%, 70%, 80%, 90%, 95%, 97%, 98%, 99%, or 100% infectivity as compared to AAV virions at the same MOI. In some embodiments, the AAV virions are wildtype AAV virions produced by a cell having wildtype AAV. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the stable cell line is conditionally capable of producing rAAV virions having a F:E ratio of no less than 0.5, 0.6, 0.7, 0.8, 0.9, 0.95, 0.97, or 0.99. In some embodiments, the rAAV virions have a F:E ratio of no less than 0.5, 0.6, 0.7, 0.8, 0.9, 0.95, 0.97, or 0.99 prior to purification. In some embodiments, at least one cell of the stable cell line is cryopreserved. In some embodiments, at least one cell of the stable cell line is in a vial, flask, syringe, or other suitable cell-storage container.

In some embodiments, a method of producing a stable cell line comprises contacting a cell to the Rep/Cap construct as described herein, and expanding the cell to produce the stable cell line. In some embodiments, a method of producing a stable cell line comprises contacting a cell to the inducible helper construct as described herein, and expanding the cell to produce the stable cell line. In some embodiments, a method of producing a stable cell line comprises contacting a cell to the Rep/Cap construct, contacting the cell to the inducible helper construct as described herein, and expanding the cell to produce the stable cell line. In some embodiments, a method of producing a stable cell line comprises contacting a cell to the Rep/Cap construct, contacting the cell to inducible helper construct as described herein, contacting the cell to the payload construct, and expanding the cell to produce the stable cell line.

### Cell Cultures and Bioreactors

In some embodiments, a cell, population of cells, or stable cell line as disclosed herein is in a cell culture. In some embodiments, a cell culture composition comprising: a) suspension-adapted cells, b) serum-free cell culture media, and c) recombinant AAV (rAAV) virions, wherein the cell culture composition is free of herpes simplex virus, baculovirus, and adenovirus, and wherein the cell culture composition is free of plasmid and transfection agent. In some embodiments, the cell culture composition is free of polyethylenimine (PEI). In some embodiments, the suspension-adapted cells are suspension-adapted mammalian cells. In some embodiments, the suspension-adapted cells are suspension-adapted HEK293 cells or derivatives thereof. In some embodiments, the suspension-adapted mammalian cells are cells from the stable cell line of as disclosed herein, the population of cells as disclosed herein, or comprise a cell as disclosed herein. In some embodiments, the cell culture composition has a prepurification rAAV concentration of no less than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, or 5×10¹⁵ viral genome (vg)/L. In some embodiments, the cell culture composition has a prepurification rAAV encapsidation ratio of no less than 0.5, 0.6, 0.7, 0.8, 0.9, 0.95, 0.97, or 0.99.

In some embodiments, rAAV virion from the stable cells as disclosed herein is produced in a bioreactor. In some embodiments, a bioreactor comprises the stable cell line as disclosed herein. In some embodiments, a bioreactor comprising the population of cells of as disclosed herein. In some embodiments, a bioreactor comprising the cell as disclosed herein. In some embodiments, a bioreactor contains the cell culture as disclosed herein. In some embodiments, the bioreactor is a 1L bioreactor. In some embodiments, the IL bioreactor has a total rAAV yield of greater than 1×10¹⁴ viral genome (vg). In some embodiments, the bioreactor is a 5L bioreactor. In some embodiments, the 5L bioreactor has a total rAAV yield of greater than 5×10¹⁴ viral genome (vg). In some embodiments, the bioreactor is a 50L bioreactor. In some embodiments, the 50L bioreactor has a total rAAV yield of greater than 5×10¹⁵ viral genome (vg). In some embodiments, the bioreactor is a 100L bioreactor. In some embodiments, the 100L bioreactor has a total rAAV yield of greater than 1×10¹⁶ viral genome (vg). In some embodiments, the bioreactor is a 500L bioreactor. In some embodiments, the 500L bioreactor has a total rAAV yield of greater than 5×1016 viral genome (vg). In some embodiments, the bioreactor is a 2000L bioreactor. In some embodiments, the 2000L bioreactor has a total rAAV yield of greater than 2×10¹⁷ viral genome (vg). In some embodiments, a bioreactor comprises a plurality of rAAV virions having a concentration of greater than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, or 5×10¹⁵ viral genome (vg)/L. In some embodiments, a bioreactor comprises a plurality of rAAV virions having a prepurification concentration of greater than 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, or 5×10¹⁵ viral genome (vg)/L. In some embodiments, the bioreactor is a 1L, 5L, 50L, 100L, 500L, or 2000L bioreactor. In some embodiments, the bioreactor is a single use bioreactor.

Compositions of rAAV

In some embodiments, the cell, population of cells, or stable cell line as disclosed herein is induced (as disclosed herein, e.g., after administration of a first and a second triggering agent in a bioreactor) to produce a plurality of rAAV virions. In some embodiments, a composition comprises a plurality of rAAV virions encapsidating a viral genome, wherein the composition has a prepurification concentration of greater than 1×10¹¹ or no less than 5×10¹¹, 1× 10¹², 5×10¹², 1×10¹³ or 1×10¹⁴ viral genomes per milliliter. In some embodiments, a composition comprises a plurality of rAAV virions encapsidating a viral genome, wherein the composition has a prepurification encapsidation ratio of no less than 0.5, 0.6, 0.7, 0.8, 0.9, 0.95, 0.97, or 0.99. In some embodiments, a composition comprises a plurality of rAAV virions encapsidating a viral genome, wherein the composition has a prepurification F:E ratio of no less than 0.5, 0.6, 0.7, 0.8, 0.9, 0.95, 0.97, or 0.99. In some embodiments, a composition comprises an rAAV virion encapsidating a viral genome, wherein the composition has an infectivity of no less than 50%, 60%, 70%, 80%, 90%, 95%, or 99% at an MOI of 1×10⁵ vg/target cell or less. In some embodiments, the rAAV virion has an increased infectivity compared an rAAV virion produced by an otherwise comparable cell capable of producing rAAV virions upon transient transfection at the same MOI. In some embodiments, the rAAV virion has at least 1%, 5%, 10%, 15%, 20%, 30%, 40%, or 50% greater infectivity compared an rAAV virion produced by an otherwise comparable cell capable of producing rAAV virions upon transient transfection at the same MOI. In some embodiments, the rAAV virion has at least 50%, 60%, 70%, 80%, 90%, 95%, 97%, 98%, 99%, or 100% infectivity as compared an AAV virion produced by a cell having wildtype AAV at the same MOI. In some embodiments, the rAAV virion has at least 50%, 60%, 70%, 80%, 90%, 95%, 97%, 98%, 99%, or 100% infectivity as compared an AAV virion produced by a cell having wildtype AAV at the same MOI. In some embodiments, the compositions further comprises a plurality of the rAAV virion. In some embodiments, the plurality of rAAV virions have a prepurification concentration of greater than 1×10¹¹ or no less than 5×10¹¹, 1×10¹², 5×10¹², 1×10¹³ or 1×10¹⁴ viral genomes per milliliter. In some embodiments, the plurality of rAAV virions have a prepurification encapsidation ratio of no less than 0.5, 0.6, 0.7, 0.8, 0.9, 0.95, 0.97, or 0.99. In some embodiments, the plurality of rAAV virions have a prepurification F:E ratio of no less than 0.5, 0.6, 0.7, 0.8, 0.9, 0.95, 0.97, or 0.99. In some embodiments, the plurality of rAAV virions have an infectivity of no less than 50%, 60%, 70%, 80%, 90%, 95%, or 99%. In some embodiments, the plurality of rAAV virions have an increased infectivity compared a plurality of rAAV virions produced by an otherwise comparable the population of cells capable of producing rAAV virions upon transient transfection at the same MOI. In some embodiments, the plurality of rAAV virions have at least 1%, 5%, 10%, 15%, 20%, 30%, 40%, or 50% greater infectivity compared a plurality of rAAV virions produced by an otherwise comparable the population of cells capable of producing rAAV virions upon transient transfection at the same MOI. In some embodiments, the plurality of rAAV virions have at least 50%, 60%, 70%, 80%, 90%, 95%, 97%, 98%, 99%, or 100% infectivity as compared a plurality of AAV virions produced by a cell having wildtype AAV at the same MOI. In some embodiments, the MOI is 1×10¹, 1×10², 2×10³, 5×10⁴, or 1×10⁵ vg/target cell. In some embodiments, the MOI is selected from a range of 1×10¹ to 1×10⁵ vg/target cell. In some embodiments, the viral genome comprises a sequence coding for a payload. In some embodiments, expression of the sequence of the payload is driven by a constitutive promoter. In some embodiments, the sequence of the payload comprises a polynucleotide sequence coding for a gene. In some embodiments, the gene codes for a selectable marker or detectable marker. In some embodiments, the gene codes for a therapeutic polypeptide or transgene. In some embodiments, the sequence of the payload comprises a polynucleotide sequence coding for a therapeutic polynucleotide. In some embodiments, the therapeutic polynucleotide is a tRNA suppressor or a guide RNA. In some embodiments, the guide RNA is a polyribonucleotide capable of binding to a protein. In some embodiments, the protein is nuclease. In some embodiments, the protein is a Cas protein, an ADAR protein, or an ADAT protein. In some embodiments, the Cas protein is catalytically inactive Cas protein. In some embodiments, the rAAV virion comprises a Cap polypeptide. In some embodiments, the Cap polypeptide is an AAV capsid protein. In some embodiments, the AAV capsid protein is VP1, VP2, or VP3. In some embodiments, a serotype of the AAV capsid protein is selected from the group consisting of AAV1, AAV2, AAV3, AAV4, AAV5, AAV6, AAV7, AAV8, AAV9, AAV 10, AAV11, AAV 12, AAV13, AAV 14, AAV 15 and AAV 16, AAV.rh8, AAV.rh10, AAV.rh20, AAV.rh39, AAV.Rh74, AAV.RHM4-1, AAV.hu37, AAV.Anc80, AAV.Anc80L65, AAV.7m8, AAV.PHP.B, AAV2.5, AAV2YF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, and AAVhu68.

In some embodiments, rAAV virions as disclosed herein are in a first composition and a second composition. In some embodiments, the first composition and the second composition have an encapsidation ratio that varies by no more than 20%, 10%, 5%, 4%, 3%, 2%, or 1%. In some embodiments, the first composition and the second composition have an F:E ratio that varies by no more than 20%, 10%, 5%, 4%, 3%, 2%, or 1%. In some embodiments, the first composition and the second composition have a concentration of viral genomes per milliliter that varies by no more than 20%, 10%, 5%, 4%, 3%, 2%, or 1%. In some embodiments, the first composition and the second composition have an infectivity that varies by no more than 20%, 10%, 5%, 4%, 3%, 2%, or 1%. In some embodiments, the first composition is a first dose and the second composition is a second dose. In some embodiments, the first composition is produced at least 1, 2, 3, 4, 5, 6, or 7 days before the second composition is produced. In some embodiments, a plurality of rAAV virions of the first composition is produced at least 1, 2, 3, 4, 5, 6, or 7 days before a plurality of rAAV virions of the second composition is produced. In some embodiments, the first composition is produced at least 1, 2, 3, 4, 5, 6, 7, 8, 9, 10, 11, or 12 months before the second composition is produced. In some embodiments, a plurality of rAAV virions of the first composition is produced at least 1, 2, 3, 4, 5, 6, 7, 8, 9, 10, 11, or 12 months before the second composition is produced. In some embodiments, the first composition is produced at least 1, 2, 3, 4, 5, 6, 7, 8, 9, 10, 11, or 12 years before the second composition is produced. In some embodiments, a plurality of rAAV virions of the first composition is produced at least 1, 2, 3, 4, 5, 6, 7, 8, 9, 10, 11, or 12 years before the second composition is produced. In some embodiments, the first composition is produced from a plurality of virions from a first bioreactor and the second composition is produced from a plurality of virions from a second bioreactor. In some embodiments, a third composition or more compositions are produced from the rAAV as disclosed herein. In some embodiments, the first composition, the second composition, and the third composition have an encapsidation ratio that varies by no more than 20%, 10%, 5%, 4%, 3%, 2%, or 1%. In some embodiments, the first composition, the second composition, and the third composition have an F:E ratio that varies by no more than 20%, 10%, 5%, 4%, 3%, 2%, or 1%. In some embodiments, the first composition, the second composition, and the third composition have a concentration of viral genomes per milliliter that varies by no more than 20%, 10%, 5%, 4%, 3%, 2%, or 1%. In some embodiments, the first composition, the second composition, and the third composition have an infectivity that varies by no more than 20%, 10%, 5%, 4%, 3%, 2%, or 1%. In some embodiments, the third composition is a third dose. In some embodiments, the third composition is produced at least 1, 2, 3, 4, 5, 6, or 7 days after the second composition is produced. In some embodiments, a plurality of rAAV virions of the third composition is produced at least 1, 2, 3, 4, 5, 6, or 7 days after a plurality of rAAV virions of the second composition is produced. In some embodiments, the third composition is produced at least 1, 2, 3, 4, 5, 6, 7, 8, 9, 10, 11, or 12 months after the second composition is produced. In some embodiments, a plurality of rAAV virions of the third composition is produced at least 1, 2, 3, 4, 5, 6, 7, 8, 9, 10, 11, or 12 months after the second composition is produced. In some embodiments, the third composition is produced at least 1, 2, 3, 4, 5, 6, 7, 8, 9, 10, 11, or 12 years after the second composition is produced. In some embodiments, a plurality of rAAV virions of the third composition is produced at least 1, 2, 3, 4, 5, 6, 7, 8, 9, 10, 11, or 12 years after the second composition is produced. In some embodiments, the third composition is produced from a plurality of virions from a third bioreactor.

### Pharmaceutical Compositions

In some embodiments, a pharmaceutical composition comprises the plurality of rAAV virions of any one of the embodiments as disclosed herein and a pharmaceutically acceptable carrier. In some embodiment, a plurality of pharmaceutical doses each independently comprise the plurality of rAAV virions of any one of the embodiments as disclosed herein and a pharmaceutically acceptable carrier. In some embodiments, the encapsidation ratio has a difference of not more than 20%, 10%, 5%, 4%, 3%, 2%, or 1% between a first dose and a second dose of a plurality of pharmaceutical doses. In some embodiments, the F:E ratio has a difference of not more than 20%, 10%, 5%, 4%, 3%, 2%, or 1% between a first dose and a second dose of a plurality of pharmaceutical doses. In some embodiments, the concentration of viral genomes has a difference of not more than 20%, 10%, 5%, 4%, 3%, 2%, or 1% between a first dose and a second dose of a plurality of pharmaceutical doses. In some embodiments, the concentration of vector genomes has a difference of not more than 20%, 10%, 5%, 4%, 3%, 2%, or 1% between a first dose and a second dose of a plurality of pharmaceutical doses. In some embodiments, the rAAV virion infectivity has a difference of not more than 20%, 10%, 5%, 4%, 3%, 2%, or 1% between a first dose and a second dose of a plurality of pharmaceutical doses.

Method of Producing rAAV

In another aspect, methods of producing rAAV from stable cell lines is provided. The method comprises adding the at least first and at least second expression triggering agents to the medium within which the stable mammalian cell lines described above are being cultured.

In another aspect, methods of producing rAAV from a cell or population of cells as described herein is provided. The method comprises adding the at least first and at least second expression triggering agents to the medium within which the cell or population of cells described above are being cultured.

In particular embodiments, the first expression triggering agent is a tetracycline. In specific embodiments, the first expression triggering agent is Dox. In particular embodiments, the second expression triggering agent is an estrogen agonist or selective estrogen receptor modulator. In specific embodiments, the second expression triggering agent is tamoxifen.

In some embodiments, the method further comprises a later step of culturing the stable mammalian cell line only in the presence of the first expression triggering agent. In some embodiments, the method further comprises a later step of culturing the cell or population of cells only in the presence of the first expression triggering agent.

In some embodiments, the method further comprises purifying rAAV from culture medium. In some embodiments, the purifying comprises performing chromatographic purification. In some embodiments, the chromatographic purification comprises using a positively charged anion exchange resin, using a negatively charged anion exchange resin, using cation exchange chromatography, using affinity chromatography, using size exclusion chromatography, or a combination thereof. In some embodiments, the chromatographic purification comprises using column chromatographic fractionation.

In some embodiments, rAAV is produced in a bioreactor as described herein.

In some embodiments, a method of inducing the cell as described herein, the population of cells as described herein, or the stable cell line as described herein comprises administering a first triggering agent to the cell, population of cells, or the stable cell line, thereby inducing expression of the Rep polypeptides, Cap polypeptides, and one or more adenoviral helper proteins, in the cell, population of cells, or stable cell line. In some embodiments, the first triggering agent binds to an activator or a repressor. In some embodiments, activation of an inducible promoter is induced. In some embodiments, the activated inducible promoter transcribes a recombinase. In some embodiments, the first triggering agent is tetracycline or cumate. In some embodiments, the tetracycline is doxycycline. The methods described herein further comprise culturing the cell, population of cells, or the stable cell line with a second triggering agent. In some embodiments, the second triggering agent is an estrogen receptor ligand. In some embodiments, the second triggering agent is a selective estrogen receptor modulator (SERM). In some embodiments, the second triggering agent is tamoxifen. In some embodiments, the second triggering agent binds to the recombinase. In some embodiments, the second triggering agent induces the recombinase to translocate to a nucleus of the cell, of a cell of the population of cells, of a cell of the stable cell lines.

In some embodiments, a method of producing rAAV virion comprises administering a first triggering agent to the cell, population of cells, or the stable cell line, administering a second triggering agent to the cell, population of cells, or stable cell line, thereby producing the rAAV virion in the cell, population of cells, or stable cell line. In some embodiments, the first triggering agent binds to an activator or a repressor. In some embodiments, activation of an inducible promoter is induced. In some embodiments, the activated inducible promoter transcribes a recombinase. In some embodiments, the first triggering agent is tetracycline or cumate. In some embodiments, the tetracycline is doxycycline. In some embodiments, the method further comprises culturing the cell, population of cells, or the stable cell line with a second triggering agent. In some embodiments, the second triggering agent is an estrogen receptor ligand. In some embodiments, the second triggering agent is a selective estrogen receptor modulator (SERM). In some embodiments, the second triggering agent is tamoxifen. In some embodiments, the second triggering agent binds to the recombinase. In some embodiments, the second triggering agent induces the recombinase to translocate to a nucleus of the cell, of a cell of the population of cells, of a cell of the stable cell lines. In some embodiments, the recombinase cuts at recombinase sites. In some embodiments, the at least one adenoviral help proteins, the Rep polypeptides, and the Cap polypeptides are expressed. In some embodiments, the Rep polypeptides and the Cap polypeptides assemble into an rAAV virion. In some embodiments, the rAAV virion encapsidates a sequence of a payload. In some embodiments, the cell, population of cells, or stable cell line do not express cytotoxic levels of Rep polypeptides prior to administration of both the first triggering agent and the second triggering agent. In some embodiments, the cell, population of cells, or stable cell line do not express cytotoxic levels of Cap polypeptides prior to administration of both the first triggering agent and the second triggering agent. In some embodiments, the cell, population of cells, or stable cell line do not express cytostatic levels of Rep polypeptides prior to administration of both the first triggering agent and the second triggering agent. In some embodiments, the average concentration of Rep polypeptides within the cell, population of cells, or stable cell line is less than the amount prior to administration of both of the first triggering agent and second triggering agent. In some embodiments, expression of Rep polypeptides and Cap polypeptides becomes constitutive after administration of both the first triggering agent and the second triggering agent. In some embodiments, the method further comprises performing at least a portion of the method in a bioreactor. In some embodiments, the bioreactor is not less than 20 L, 30, L, 40 L, 50 L, 100 L, 250 L, 300 L, or 500 L.

In some embodiments, the method further comprises producing the rAAV virions in a plurality of batches. In some embodiments, the method further comprises producing the rAAV virions having a difference in the encapsidation ratio of not more than 20%, 15%, 10%, 5%, 3%, 2%, or 1% between a first batch and a second batch. In some embodiments, the method further comprises producing the rAAV virions having a difference in the F:E ratio of not more than 20%, 15%, 10%, 5%, 3%, 2%, or 1% between a first batch and a second batch. In some embodiments, the method further comprises producing the rAAV virions having a difference in the concentration of viral genomes of not more than 20%, 15%, 10%, 5%, 3%, 2%, or 1% between a first batch and a second batch. In some embodiments, the method further comprises producing the rAAV virions having a difference in the concentration of vector genomes of not more than 20%, 15%, 10%, 5%, 3%, 2%, or 1% between a first batch and a second batch. In some embodiments, the method further comprises producing the rAAV virions having a difference in infectivity of not more than 20%, 15%, 10%, 5%, 3%, 2%, or 1% between a first batch and a second batch. In some embodiments, the method further comprises performing the method according to good manufacturing practice (GMP) standards. In some embodiments, the method further comprises performing the method in a GMP facility. In some embodiments, the method further comprises comprising culturing the cells in a culture medium and collecting a portion of the plurality of rAAV virions from the culture medium. In some embodiments, the method further comprises purifying at least some of the plurality of rAAV virions collected from the culture medium to obtain a purified rAAV population. In some embodiments, the purifying comprises performing chromatographic purification. In some embodiments, the chromatographic purification comprises using a positively charged anion exchange resin, using a negatively charged anion exchange resin, using cation exchange chromatography, using affinity chromatography, using size exclusion chromatography, or a combination thereof. In some embodiments, the chromatographic purification comprises using column chromatographic fractionation.

In some embodiments, an rAAV virion is made by the methods as disclosed herein. In some embodiments, a composition comprising a plurality of rAAV virions is made by the methods as disclosed herein. In some embodiments, the rAAV virion produced as disclosed herein has increased infectivity compared to an rAAV virion produced by comparable transient transfection methods.

### Methods of Treatment

In another aspect, methods of treatment are provided. In various embodiments, the method comprises administering rAAV produced by the process described above to a patient in need thereof. In some embodiments, the administering is by intravenous administration, intramuscular administration, intrathecal administration, intracisternal administration, or administration via brain surgery.

In some embodiments, a method of treating a condition or disorder comprises administering a therapeutically effective amount of the pharmaceutical composition of as disclosed herein to a patient in need thereof. In some embodiments, the disorder is a monogenic disorder. In some embodiments, the treatment results in at least one undesirable side effect and wherein the undesirable side effect is reduced relative to administering a daily dose that deviates more than 50%, 40%, 30%, 30%, 15%, 10%, 5%, or 2% from an expected dose. In some embodiments, the administering is by injection. In some embodiments, the injection is an infusion. In some embodiments, the daily dose is administered to the patient once. In some embodiments, the daily dose is administered to the patient two or more times. In some embodiments, the treatment results in at least one undesirable side effect and wherein the undesirable side effect is reduced relative to administering a plurality of rAAV virions produced from a triple transfection method.

In some embodiments, the methods reduce the immunogenicity of a dose of rAAV having a predetermined number of viral genomes (VG) as compared to the same rAAV VG dose prepared by transient triple transfection. In some embodiments, the immunogenicity is measured by the titer or concentration of neutralizing antibodies in a subject. In some embodiments, a concentration of rAAV virion neutralizing antibody in the blood serum of the patient is reduced relative to a concentration of rAAV virion neutralizing antibody in the blood serum of a patient after administering a plurality of rAAV virions produced from a triple transfection method. In some embodiments, the concentration of rAAV virion neutralizing antibodies is measured by an ELISA assay.

In some embodiments, the methods reduce the number or intensity of adverse effects caused by administering a dose of rAAV having a predetermined number of viral genomes (VG) as compared to the same rAAV VG dose prepared by transient triple transfection. In some embodiments, the methods reduce the number of adverse effects. In some embodiments, the predetermined number of VG in a dose is no greater than 3×10¹⁴ vg/kg. In some embodiments, the predetermined number of VG in a dose is no greater than 1×10¹⁴ vg/kg. In some embodiments, the predetermined number of VG in a dose is no greater than 5×10¹³ vg/kg. In some embodiments, the methods reduce the intensity of adverse effects. In some embodiments, the methods reduce both the number and the intensity of adverse events.

In some embodiments, a method of administering a dose of rAAV virions having a predetermined number of viral genomes (VG) to a subject with reduced number or intensity of adverse effects as compared to administration of the same rAAV VG dose prepared by transient triple transfection comprises: administering a dose of rAAV produced in the cell as disclosed herein, the population of cells disclosed herein, or the stable cells as disclosed herein. In some embodiments, the adverse effect is selected from the group consisting of: liver dysfunction, liver inflammation, gastrointestinal infection, vomiting, bacterial infection, sepsis, increases in troponin levels, decreases in red blood cell counts, decreases in platelet counts, activation of the complement immune system response, acute kidney injury, cardio-pulmonary insufficiency, and death. In some embodiments, the adverse effect is an increase in serum levels of one or more proinflammatory cytokines. In some embodiments, the adverse effect is an increase in serum levels of one or more of interferon gamma (IFNγ), interleukin 1β (IL-1β), and interleukin 6 (IL-6).

In another aspects, a method of repeatedly administering a dose of rAAV to a subject in need thereof are provided. In some embodiments, the method comprises administering a first dose of rAAV produced by the cell lines and the processes described above, and then administering at least a second dose of rAAV produced by the cell lines and the processes described above. In some embodiments, the method comprises administering a first dose and a second dose of rAAV produced by the cell lines and the processes described above. In some embodiments, the method comprises administering a first dose, a second dose, and a third dose of rAAV produced by the cell lines and the processes described above. In some embodiments, the method comprises administering more than three doses of rAAV produced by the cell lines and the processes described above. In some embodiments, the first dose of rAAV and the at lease second dose of rAAV are administered through the same route of administration. In some embodiments, the first dose of rAAV and the at least second dose of rAAV are administered through different routes of administration. In some embodiments, the route of administration is intravenous administration, intramuscular administration, intrathecal administration, intracisternal administration, or administration via brain surgery.

In some embodiments, a method of treating a condition or disorder comprises administering a first therapeutically effective amount of the pharmaceutical composition of as disclosed herein having a predetermined number of viral genomes to a patient in need thereof and a second therapeutically effective amount of the pharmaceutical composition as disclosed herein having the predetermined number of viral genomes to the patient in need thereof. In some embodiments, the first therapeutically effective amount and the second therapeutically effective amount vary by no more than 1%, 5%, 10%, or 15%.

### Kits

In another aspect, components or embodiments described herein for the system are provided in a kit. For example, any of the plasmids, as well as the mammalian cells, related buffers, media, triggering agents, or other components related to cell culture and virion production can be provided, with optional components frozen and packaged as a kit, alone or along with separate containers of any of the other agents and optional instructions for use. In some embodiments, the kit may comprise culture vessels, vials, tubes, or the like.

The methods for producing and packaging recombinant vectors in desired AAV capsids to produce the rAAVs are not meant to be limiting and other suitable methods will be apparent to the skilled artisan.

### Aspects of the Invention

The below items disclose various aspects of the invention. Each of the aspects described below can be combined with other aspects and embodiments disclosed elsewhere herein, including the claims, where the combinations are clearly compatible. For example, described herein are three exemplary, non-limiting, constructs, referred to as “construct 1”, “construct 2” and “construct 3”. The disclosure provided herein describes these constructs in specific and general detail.

In the following aspects, the first recombinant nucleic acid sequence encoding an AAV Rep protein and an AAV Cap protein corresponds to the specific and general disclosures of “construct 1” provided herein. It is intended that any aspects described below relating to the first recombinant nucleic acid may be combined with any of the specific and general disclosures of “construct 1” provided herein where the combinations are clearly compatible.

In the following aspects, the second recombinant nucleic acid sequence encoding one or more adenoviral helper proteins corresponds to the specific and general disclosures of “construct 2” provided herein. It is intended that any aspects described below relating to the second recombinant nucleic acid may be combined with any of the specific and general disclosures of “construct 2” provided herein where the combinations are clearly compatible.

In the following aspects, the third recombinant nucleic acid sequence encoding a payload corresponds to the specific and general disclosures of “construct 3” provided herein. It is intended that any aspects described below relating to the third recombinant nucleic acid may be combined with any of the specific and general disclosures of “construct 3” provided herein where the combinations are clearly compatible.

It is intended that any aspects and disclosures provided herein relating to the first, second and third recombinant nucleic acids, and relating to the specific and general disclosures of constructs 1, 2 and 3 may be combined together where the combinations are clearly compatible.

### NUMBERED EMBODIMENTS

1. A cell for inducibly producing recombinant adenovirus associated virus (rAAV) virions, the cell comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by
(i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence; and a first constitutive promoter operably linked to a sequence encoding a first selectable marker; and
a second polynucleotide construct comprising an inducible promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding one or more AAV helper proteins, wherein the inducible promoter is not operably linked to the sequence encoding the one or more AAV helper proteins; a second constitutive promoter operably linked to a sequence encoding an activator, wherein the cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the one or more AAV helper proteins.
2. The cell of embodiment 1, further comprising a third polynucleotide construct comprising a promoter operably linked to a sequence encoding a payload and a third selectable marker, wherein the sequence encoding the payload is flanked by a 5′ AAV inverted terminal repeat (5′ ITR) and a 3′ AAV inverted terminal repeat (3′ ITR); optionally, wherein the sequence encoding the payload is flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity any one of SEQ ID NO: 146-SEQ ID NO: 153.
3. The cell of embodiment 2, wherein a terminal resolution site in the 3′ ITR is deleted.
4. The cell of embodiment 2 or 3, wherein the third polynucleotide construct further comprises a spacer between the 5′ ITR and the sequence encoding the third selectable marker or a spacer between the sequence encoding the third selectable marker and the 3′ ITR, or a combination thereof.
5. The cell of embodiment 4, wherein the spacer ranges in length from 500 base pairs to 5000 base pairs.
6. The cell of any one of embodiments 1-5, wherein the coding sequence encoding the stop signaling sequence of the first polynucleotide construct further encodes a protein marker that comprises the stop signaling sequence.
7. The cell of any one of embodiments 1-6, wherein the cell further comprises an adenovirus E1A protein and E1B protein, and the one or more AAV helper proteins expressed by the second polynucleotide construct are an adenovirus E2A protein and E4 protein.
8. The cell of any one of embodiments 1-6, wherein the cell further comprises an adenovirus E2A protein and E4 protein, and the one or more AAV helper proteins expressed by the second polynucleotide construct are an adenovirus E1A protein and E1B protein.
9. The cell of any one of embodiments 1-6, further comprising a fourth polynucleotide construct comprising an inducible or constitutive promoter operably linked to a sequence encoding one or more helper proteins.
10. The cell of embodiment 9, wherein the one or more AAV helper proteins expressed by the second polynucleotide construct are an adenovirus E2A protein and E4 protein and the one or more AAV helper proteins expressed by the fourth polynucleotide construct are an adenovirus E1A protein and E1B protein.
11. The cell of embodiment 9, wherein the one or more AAV helper proteins expressed by the second polynucleotide construct are an adenovirus E1A protein and E1B protein and the one or more AAV helper proteins expressed by the fourth polynucleotide construct are an adenovirus E2A protein and E4 protein.
12. The cell of any one of embodiments 1-11, wherein the sequence coding for one or more AAV helper proteins comprises a bicistronic open reading frame encoding two AAV helper proteins.
13. The cell of embodiment 12, wherein the two AAV helper proteins are E2A and E4 or E1A and E1B.
14. The cell of embodiment 12 or 13, wherein the bicistronic open reading frame comprises an internal ribosome entry site (IRES) or a peptide 2A (P2A) sequence.
15. The cell of any one of embodiments 1-14, wherein transcription of the AAV Rep coding sequences and the sequence encoding one or more AAV capsid proteins are driven by native AAV promoters.
16. The cell of embodiment 15, wherein transcription of the AAV Rep coding sequences is driven by the P5 and P19 native AAV promoters, and transcription of the sequence encoding the one or more AAV capsid proteins is driven by the P40 native AAV promoter.
17. The cell of any one of embodiments 1-14, wherein transcription of the AAV Rep coding sequences is driven by an inducible promoter.
18. The cell of any one of embodiments 1-17, wherein transcription of the sequence encoding the one or more AAV capsid proteins is driven by an inducible promoter.
19. The cell of any one of embodiments 1-18, wherein the AAV capsid proteins comprise VP1, VP2, and VP3.
20. The cell of embodiment 19, wherein the third sequence of the first polynucleotide construct further comprises a first inducible promoter operably linked to the sequence encoding the VP1 capsid protein, a second inducible promoter operably linked to the sequence encoding the VP2 capsid protein, or a third inducible promoter operably linked to the sequence encoding the VP3 capsid protein, or a combination thereof.
21. The cell of embodiment 20, wherein the third sequence of the first polynucleotide construct further comprises a first inducible promoter operably linked to the sequence encoding the VP1 capsid protein, a second inducible promoter operably linked to the sequence encoding the VP2 capsid protein, and a third inducible promoter operably linked to the sequence encoding the VP3 capsid protein.
22. The cell of any one of embodiments 1-21, wherein the cell is a mammalian cell.
23. The cell of embodiment 22, wherein the mammalian cell is a HEK293 cell.
24. The cell of embodiment 23, wherein the HEK293 cell is DHFR-deficient or GS-deficient.
25. The cell of any one of embodiments 1-24, wherein the first polynucleotide construct, the second polynucleotide construct, or the combination thereof are integrated into the nuclear genome of the cell.
26. The cell of embodiment 25, wherein the first polynucleotide construct, the second polynucleotide construct, or a combination thereof are integrated into the nuclear genome of the cell using a transposon system, a clustered regularly interspersed short palindromic repeats (CRISPR) system, or a site-specific recombinase.
27. The method of embodiment 26 wherein the transposon system is a Tol2, piggyBac, or Sleeping Beauty transposon system.
28. The cell of embodiment 25, wherein the first polynucleotide construct, the second polynucleotide construct, or the combination thereof are provided by a lentivirus vector that integrates into the nuclear genome of the cell.
29. The cell of any one of embodiments 1-24, wherein the first polynucleotide construct, the second polynucleotide construct, or the combination thereof are not integrated into the nuclear genome of the cell.
30. The cell of embodiment 29, wherein the first polynucleotide construct and the second polynucleotide construct further comprise Epstein-Barr virus (EBV) sequences to stably maintain the constructs extrachromosomally.
31. The cell of any one of embodiments 1-30, wherein the inducible promoter in the second polynucleotide construct is selected from the group consisting of a tetracycline-inducible promoter, an ecdysone-inducible promoter, and a cumate-inducible promoter, 32. The cell of embodiment 31, wherein the tetracycline-inducible promoter comprises a tetracycline-responsive promoter element (TRE).
33. The cell of embodiment 32, wherein the TRE comprises Tet operator (tetO) sequence concatemers fused to a minimal promoter.
34. The cell of embodiment 33, wherein the minimal promoter is a human cytomegalovirus promoter or comprises the sequence as set forth in one of SEQ ID NOs: 63-68.
35. The cell of any one of embodiments 31-34, wherein the TRE comprises the sequence as set forth in one of SEQ ID NOs: 22, 46-48, and 50-62.
36. The cell of any one of embodiments 31-35, wherein the activator comprises the sequence as set forth in one of SEQ ID NOs: 21, 40-45, and 69-86, or variants thereof.
37. The cell of any one of embodiments 31-36, wherein the triggering agent for inducing the tetracycline-inducible promoter is tetracycline or doxycycline.
38. The cell of embodiment 37, wherein the activator is a reverse tetracycline-controlled transactivator (rTA) comprising a Tet Repressor binding protein (TetR) fused to a VP16 transactivation domain, and the triggering agent is tetracycline or doxycycline.
39. The cell of embodiment 31, wherein the triggering agent for inducing the ecdysone-inducible promoter is ecdysone or ponasterone.
40. The cell of embodiment 31, wherein the triggering agent for inducing the cumate-inducible promoter is cumate.
41. The cell of embodiment 40, wherein the cumate-inducible promoter comprises a cumate operator sequence.
42. The cell of any one of embodiments 1-41, wherein the inducible recombinase is fused to an estrogen response element (ER) and translocates to the nucleus in the presence of tamoxifen.
43. The cell of any one of embodiments 1-42, wherein the first selectable marker encoded by the first polynucleotide construct comprises:
a C-terminal fragment of the mammalian DHFR (Cter-DHFR) fused to a leucine zipper peptide, and the selectable marker encoded by the third polynucleotide construct comprises an N-terminal fragment of the mammalian DHFR (Nter-DHFR) fused to a leucine zipper peptide, or an N-terminal fragment of the mammalian DHFR (Nter-DHFR) fused to a leucine zipper peptide, and the selectable marker encoded by the third polynucleotide construct comprises a C-terminal fragment of the mammalian DHFR (Cter-DHFR) fused to a leucine zipper peptide.
44. The cell of any one of embodiments 1-42, wherein the selectable marker encoded by the first polynucleotide construct, second polynucleotide construct, or third polynucleotide construct is an auxotrophic protein or antibiotic resistance protein; optionally, wherein the auxotrophic protein is glutamine synthetase (GS), thymidylate synthase (TYMS), phenylalanine hydroxylase (PAH), or dihydrofolate reductase (DHFR); optionally, wherein the antibiotic resistance protein is blasticidin resistance or puromycin resistance; optionally, wherein the selectable marker is an N-terminal fragment of the auxotrophic protein or antibiotic resistance protein fused to a N-terminal fragment of a split intein; optionally, wherein the an N-terminal fragment of the auxotrophic protein or antibiotic resistance protein fused to a N-terminal fragment of a split intein is any one of SEQ ID NO: 91, 93, 95, 97, 113, 115, 117, 119, 121, 124, 126, 128, 130, or 137; optionally, wherein the selectable marker is a C-terminal fragment of a split intein fused to an C-terminal fragment of the auxotrophic protein or antibiotic resistance protein; optionally, wherein the C-terminal fragment of a split intein fused to an C-terminal fragment of the auxotrophic protein or antibiotic resistance protein comprises any one of SEQ ID NO: 92, 93, 94, 96, 98, 114, 116, 118, 120, 122, 125, 127, 129, 131, or 141.
45. The cell of embodiment 44, wherein the selectable marker encoded by the first polynucleotide construct comprises a first antibiotic resistance protein, the selectable marker encoded by the second polynucleotide construct comprises a second antibiotic resistance protein, and the selectable marker encoded by the third polynucleotide construct comprises a third antibiotic resistance protein, and wherein the first antibiotic resistance protein, the second antibiotic resistance protein, and the third antibiotic resistance protein are different.
46. The cell of any one of embodiments 1-45, wherein the recombination sites in the first polynucleotide construct and the second polynucleotide construct are lox sites and the recombinase is a cre recombinase or wherein the recombination sites in the first polynucleotide construct and the second polynucleotide are flippase recognition target (FRT) sites and the recombinase is a flippase (Flp) recombinase.
47. The cell of any one of embodiments 1-46, wherein upon expression of the inducible recombinase,
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein; and recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins.
48. The cell of any one of embodiments 2-47, wherein the sequence encoding the payload comprises a reporter gene, a therapeutic gene, or a transgene encoding a protein of interest; optionally, wherein the sequence encoding the payload a sequence encoding progranulin.
49. The cell of any one of embodiments 2-47, wherein the sequence encoding the payload comprises a suppressor tRNA, a guide RNA, or a homology region for homology-directed repair.
50. The cell of any one of embodiments 1-49, wherein the second polynucleotide construct further comprises an insert comprising a sequence encoding VA-RNA; or optionally, wherein a fifth construct comprises an insert comprising a sequence encoding VA-RNA.
51. The cell of embodiment 50, wherein the VA-RNA is wild-type VA-RNA or VA-RNA comprising one or more mutations in the VA-RNA internal promoter.
52. The cell of embodiment 50 or 51, wherein the insert comprises:
a first part of a second constitutive promoter and a second part of a second constitutive promoter separated by a second excisable element comprising a fifth recombination site and a sixth recombination site flanking a stuffer sequence, wherein the fifth and sixth recombination sites are oriented in the same direction, and
a VA-RNA coding sequence, wherein excision of the second excisable element by the inducible recombinase generates a functional complete second constitutive promoter operably linked to the VA-RNA coding sequence to allow expression of the VA-RNA.
53. The cell of embodiment 52, wherein the first part of the second constitutive promoter comprises a distal sequence element (DSE) of an RNA polymerase III promoter, and the second part of the second constitutive promoter comprises a proximal sequence element (PSE) of an RNA polymerase III promoter.
54. The cell of embodiment 52, wherein the first part of the second constitutive promoter comprises a distal sequence element (DSE) of a U6 promoter, and the second part of the second constitutive promoter comprises a proximal sequence element (PSE) of a U6 promoter.
55. The cell of embodiment 52, wherein the first part of the second constitutive promoter comprises a distal sequence element (DSE) of a U7 promoter, and the second part of the second constitutive promoter comprises a proximal sequence element (PSE) of a U7 promoter.
56. The cell of any one of embodiments 51-55, wherein the VA-RNA comprises a G16A mutation or a G60A mutation, or a combination thereof.
57.

The cell of any one of embodiments 1-56, wherein the first polynucleotide construct further comprises:
(i) a first spacer segment and a second spacer segment flanking the excisable element, wherein the first part of the AAV Rep coding sequence and the second part of the AAV Rep coding sequence are separated by the first spacer segment, the excisable element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site; or
(ii) a first spacer segment and a second spacer segment flanking the inversible element, wherein the first part of the AAV Rep coding sequence and the second part of the AAV Rep coding sequence are separated by the first spacer segment, the inversible element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site.
58. The cell of embodiment 57, wherein first part of the AAV Rep coding sequence comprises a p5 internal promoter and a p19 internal promoter, and the second part of the AAV Rep coding sequence comprises a p40 internal promoter.
59. The cell of embodiment 58, wherein the excisable spacer is inserted at an insertion site between the p19 internal promoter and the p40 internal promoter of the AAV Rep coding sequence.
60. The cell of embodiment 59, wherein the insertion site is between a CAG and a G, a CAG and an A, an AAG and a G, and an AAG and an A.
61. The cell of any one of embodiments 1-60, wherein the inducible recombinase is fused to an estrogen response element (ER) and translocates to the nucleus in the presence of tamoxifen.
62. The cell of any one of embodiments 47-61, wherein the complete AAV Rep coding sequence comprises an intron.
63. A vector system for inducibly producing recombinant adenovirus associated virus (rAAV) virions, the vector system comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence; and a first constitutive promoter operably linked to a sequence encoding a first selectable marker; and
a second polynucleotide construct comprising an inducible promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding one or more AAV helper proteins, wherein the inducible promoter is not operably linked to the sequence encoding the one or more AAV helper proteins; a second constitutive promoter operably linked to a sequence encoding an activator, wherein a cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the one or more AAV helper proteins.
64. The vector system of embodiment 63, further comprising a third polynucleotide construct comprising a promoter operably linked to a sequence encoding a payload and a third selectable marker, wherein the sequence encoding the payload is flanked by a 5′ AAV inverted terminal repeat (5′ ITR) and a 3′ AAV inverted terminal repeat (3′ ITR)); optionally, wherein the sequence encoding the payload is flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to any one of SEQ ID NO: 146-SEQ ID NO: 153.
65. The vector system of embodiment 64, wherein a terminal resolution site in the 3′ ITR is deleted.
66. The vector system of embodiment 64 or 65, wherein the third polynucleotide construct wherein the third polynucleotide construct further comprises a spacer between the 5′ ITR and the sequence encoding the third selectable marker or a spacer between the sequence encoding the third selectable marker and the 3′ ITR, or a combination thereof.
67. The vector system of embodiment 66, wherein the spacer ranges in length from 500 base pairs to 5000 base pairs.
68. The vector system of any one of embodiments 63-67, wherein the coding sequence encoding the stop signaling sequence of the first polynucleotide construct further encodes a protein marker that comprises the stop signaling sequence.
69. The vector system of any one of embodiments 63-68, wherein the one or more AAV helper proteins expressed by the second polynucleotide construct are an adenovirus E2A protein and E4 protein.
70. The vector system of any one of embodiments 63-68, wherein the one or more AAV helper proteins expressed by the second polynucleotide construct are an adenovirus E1A protein and E1B protein.
71. The vector system of any one of embodiments 63-70, further comprising a fourth polynucleotide construct comprising an inducible or constitutive promoter operably linked to a sequence encoding one or more helper proteins.
72. The vector system of embodiment 71, wherein the one or more AAV helper proteins expressed by the second polynucleotide construct are an adenovirus E2A protein and E4 protein and the one or more AAV helper proteins expressed by the fourth polynucleotide construct are an adenovirus E1A protein and E1B protein.
73. The vector system of embodiment 71, wherein the one or more AAV helper proteins expressed by the second polynucleotide construct are an adenovirus E1A protein and E1B protein and the one or more AAV helper proteins expressed by the fourth polynucleotide construct are an adenovirus E2A protein and E4 protein.
74. The vector system of any one of embodiments 63-73, wherein the sequence coding for one or more AAV helper proteins comprises a bicistronic open reading frame encoding two AAV helper proteins.
75. The vector system of embodiment 74, wherein the two AAV helper proteins are E2A and E4 or E1A and E1B.
76. The vector system of embodiment 74 or 75, wherein the bicistronic open reading frame comprises an internal ribosome entry site (IRES) or a peptide 2A (P2A) sequence.
77. The vector system of any one of embodiments 63-76, wherein transcription of the AAV Rep coding sequences and the sequence encoding one or more AAV capsid proteins are driven by native AAV promoters.
78. The vector system of embodiment 77, wherein transcription of the AAV Rep coding sequences is driven by the P5 and P19 native AAV promoters, and transcription of the sequence encoding the one or more AAV capsid proteins is driven by the P40 native AAV promoter.
79. The vector system of any one of embodiments 63-76, wherein transcription of the AAV Rep coding sequences is driven by an inducible promoter.
80. The vector system of any one of embodiments 63-79, wherein transcription of the sequence encoding the one or more AAV capsid proteins is driven by an inducible promoter.
81. The vector system of any one of embodiments 63-80, wherein the AAV capsid proteins comprise VP1, VP2, and VP3.
82. The vector system of embodiment 81, wherein the third sequence of the first polynucleotide construct further comprises a first inducible promoter operably linked to the sequence encoding the VP1 capsid protein, a second inducible promoter operably linked to the sequence encoding the VP2 capsid protein, or a third inducible promoter operably linked to the sequence encoding the VP3 capsid protein, or a combination thereof.
83. The vector system of embodiment 82, wherein the third sequence of the first polynucleotide construct further comprises a first inducible promoter operably linked to the sequence encoding the VP1 capsid protein, a second inducible promoter operably linked to the sequence encoding the VP2 capsid protein, and a third inducible promoter operably linked to the sequence encoding the VP3 capsid protein.
84. The vector system of any one of embodiments 63-83, wherein the first polynucleotide construct, the second polynucleotide construct, or the combination thereof are integrated into the nuclear genome of a cell.
85. The vector system of embodiment 84, wherein the first polynucleotide construct, the second polynucleotide construct, or a combination thereof are integrated into the nuclear genome of the cell using a transposon system, a clustered regularly interspersed short palindromic repeats (CRISPR) system, or a site-specific recombinase.
86. The vector system of embodiment 85 wherein the transposon system is a Tol2, piggyBac, or Sleeping Beauty transposon system.
87. The vector system of embodiment 84, wherein the first polynucleotide construct, the second polynucleotide construct, or the combination thereof are provided by a lentivirus vector that integrates into the nuclear genome of a cell.
88. The vector system of any one of embodiments 63-83, wherein the first polynucleotide construct, the second polynucleotide construct, or the combination thereof are not integrated into the nuclear genome of a cell.
89. The vector system of embodiment 88, wherein the first polynucleotide construct and the second polynucleotide construct further comprise Epstein-Barr virus (EBV) sequences to stably maintain the constructs extrachromosomally.
90. The vector system of any one of embodiments 63-89, wherein the inducible promoter in the second polynucleotide construct is selected from the group consisting of a tetracycline-inducible promoter, an ecdysone-inducible promoter, and a cumate-inducible promoter, 91. The vector system of embodiment 90, wherein the tetracycline-inducible promoter comprises a tetracycline-responsive promoter element (TRE).
92. The vector system of embodiment 91, wherein the TRE comprises Tet operator (tetO) sequence concatemers fused to a minimal promoter.
93. The vector system of embodiment 92, wherein the minimal promoter is a human cytomegalovirus promoter or comprises the sequence as set forth in one of SEQ ID NOs: 63-68.
94. The vector system of any one of embodiments 90-93, wherein the TRE comprises the sequence as set forth in one of SEQ ID NOs: 22, 46-48, and 50-62.
95. The vector system of any one of embodiments 90-94, wherein the activator comprises the sequence as set forth in one of SEQ ID NOs: 21, 40-45, and 69-86, or variants thereof.
96. The vector system of any one of embodiments 90-95, wherein the triggering agent for inducing the tetracycline-inducible promoter is tetracycline or doxycycline.
97. The vector system of embodiment 96, wherein the activator is a reverse tetracycline-controlled transactivator (rTA) comprising a Tet Repressor binding protein (TetR) fused to a VP16 transactivation domain, and the triggering agent is tetracycline or doxycycline.
98. The vector system of embodiment 90, wherein the triggering agent for inducing the ecdysone-inducible promoter is ecdysone or ponasterone.
99. The vector system of embodiment 90, wherein the triggering agent for inducing the cumate-inducible promoter is cumate.
100. The vector system of embodiment 99, wherein the cumate-inducible promoter comprises a cumate operator sequence.
101. The vector system of any one of embodiments 63-100, wherein the inducible recombinase is fused to an estrogen response element (ER) and translocates to the nucleus in the presence of tamoxifen.
102. The vector system of any one of embodiments 63-101, wherein the first selectable marker encoded by the first polynucleotide construct comprises:
a C-terminal fragment of the mammalian DHFR (Cter-DHFR) fused to a leucine zipper peptide, and the selectable marker encoded by the third polynucleotide construct comprises an N-terminal fragment of the mammalian DHFR (Nter-DHFR) fused to a leucine zipper peptide, or an N-terminal fragment of the mammalian DHFR (Nter-DHFR) fused to a leucine zipper peptide, and the selectable marker encoded by the third polynucleotide construct comprises a C-terminal fragment of the mammalian DHFR (Cter-DHFR) fused to a leucine zipper peptide.
103. The vector system of any one of embodiments 63-101, wherein the selectable marker encoded by the first polynucleotide construct, second polynucleotide construct, or third polynucleotide construct is an auxotrophic protein or antibiotic resistance protein; optionally, wherein the auxotrophic protein is glutamine synthetase (GS), thymidylate synthase (TYMS), phenylalanine hydroxylase (PAH), or dihydrofolate reductase (DHFR); optionally, wherein the antibiotic resistance protein is blasticidin resistance or puromycin resistance; optionally, wherein the selectable marker is an N-terminal fragment of the auxotrophic protein or antibiotic resistance protein fused to a N-terminal fragment of a split intein; optionally, wherein the an N-terminal fragment of the auxotrophic protein or antibiotic resistance protein fused to a N-terminal fragment of a split intein is any one of SEQ ID NO: 91, 93, 95, 97, 113, 115, 117, 119, 121, 124, 126, 128, 130, or 137; optionally, wherein the selectable marker is a C-terminal fragment of a split intein fused to an C-terminal fragment of the auxotrophic protein or antibiotic resistance protein; optionally, wherein the C-terminal fragment of a split intein fused to an C-terminal fragment of the auxotrophic protein or antibiotic resistance protein comprises any one of SEQ ID NO: 92, 93, 94, 96, 98, 114, 116, 118, 120, 122, 125, 127, 129, 131, or 141.
104. The vector system of embodiment 103, wherein the selectable marker encoded by the first polynucleotide construct comprises a first antibiotic resistance protein, the selectable marker encoded by the second polynucleotide construct comprises a second antibiotic resistance protein, and the selectable marker encoded by the third polynucleotide construct comprises a third antibiotic resistance protein, and wherein the first antibiotic resistance protein, the second antibiotic resistance protein, and the third antibiotic resistance protein are different.
105. The vector system of any one of embodiments 63-104, wherein the recombination sites in the first polynucleotide construct and the second polynucleotide construct are lox sites and the recombinase is a cre recombinase or wherein the recombination sites in the first polynucleotide construct and the second polynucleotide are flippase recognition target (FRT) sites and the recombinase is a flippase (Flp) recombinase.
106. The vector system of any one of embodiments 63-105, wherein upon expression of the inducible recombinase,
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein; and recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins.
107. The vector system of any one of embodiments 64-106, wherein the sequence encoding the payload comprises a reporter gene, a therapeutic gene, or a transgene encoding a protein of interest; optionally, wherein the sequence encoding the payload comprises a sequence encoding progranulin.
108. The vector system of any one of embodiments 64-106, wherein the sequence encoding the payload comprises a suppressor tRNA, a guide RNA or a homology region for homology-directed repair.
109. The vector system of any one of embodiments 63-108, wherein the second polynucleotide construct further comprises an insert comprising VA-RNA or a fourth construct comprises an insert comprising VA-RNA.
110. The vector system of embodiment 109, wherein the VA-RNA is wild-type VA-RNA or VA-RNA comprising one or more mutations in the VA-RNA internal promoter.
111. The vector system of embodiment 108 or 109, wherein the insert comprises:
a first part of a second constitutive promoter and a second part of a second constitutive promoter separated by a second excisable element comprising a fifth recombination site and a sixth recombination site flanking a stuffer sequence, wherein the fifth and sixth recombination sites are oriented in the same direction, and
a VA-RNA coding sequence, wherein excision of the second excisable element by the inducible recombinase generates a functional complete second constitutive promoter operably linked to the VA-RNA coding sequence to allow expression of the VA-RNA.
112. The vector system of embodiment 111, wherein the first part of the second constitutive promoter comprises a distal sequence element (DSE) of an RNA polymerase III promoter, and the second part of the second constitutive promoter comprises a proximal sequence element (PSE) of an RNA polymerase III promoter.
113. The vector system of embodiment 111, wherein the first part of the second constitutive promoter comprises a distal sequence element (DSE) of a U6 promoter, and the second part of the second constitutive promoter comprises a proximal sequence element (PSE) of a U6 promoter.
114. The vector system of embodiment 111, wherein the first part of the second constitutive promoter comprises a distal sequence element (DSE) of a U7 promoter, and the second part of the second constitutive promoter comprises a proximal sequence element (PSE) of a U7 promoter.
115. The vector system of any one of embodiments 109-114, wherein the VA-RNA comprises a G16A mutation or a G60A mutation, or a combination thereof.
116. The vector system of any one of embodiments 63-115, wherein the first polynucleotide construct further comprises:
(i) a first spacer segment and a second spacer segment flanking the excisable element, wherein the first part of the AAV Rep coding sequence and the second part of the AAV Rep coding sequence are separated by the first spacer segment, the excisable element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site; or
(ii) a first spacer segment and a second spacer segment flanking the inversible element, wherein the first part of the AAV Rep coding sequence and the second part of the AAV Rep coding sequence are separated by the first spacer segment, the inversible element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site.
117. The vector system of embodiment 116, wherein first part of the AAV Rep coding sequence comprises a p5 internal promoter and a p19 internal promoter, and the second part of the AAV Rep coding sequence comprises a p40 internal promoter.
118. The vector system of embodiment 117, wherein the excisable spacer is inserted at an insertion site between the p19 internal promoter and the p40 internal promoter of the AAV Rep coding sequence.
119. The vector system of embodiment 118, wherein the insertion site is between a CAG and a G, a CAG and an A, an AAG and a G, and an AAG and an A.
120. The vector system of any one of embodiments 63-119, wherein the inducible recombinase is fused to an estrogen response element (ER) and translocates to the nucleus in the presence of tamoxifen.
121. The vector system of any one of embodiments 63-120, wherein the complete AAV Rep coding sequence comprises an intron.
122. A method of generating a cell line for inducibly producing recombinant AAV (rAAV) virions comprising a payload, the method comprising:
introducing into a cell a first polynucleotide construct comprising a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence; and a first constitutive promoter operably linked to a sequence encoding a first selectable marker;
selecting for cells expressing the first selectable marker;
introducing into a cell a second polynucleotide construct comprising an inducible promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding one or more AAV helper proteins, wherein the inducible promoter is not operably linked to the sequence encoding the one or more AAV helper proteins; a second constitutive promoter operably linked to a sequence encoding an activator, wherein the cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
selecting for cells expressing the second selectable marker;
introducing into a cell a third polynucleotide construct comprising a constitutive promoter operably linked to a sequence encoding a payload and a third selectable marker, wherein the sequence encoding the payload is flanked by a 5′ AAV inverted terminal repeat (5′ ITR) and a 3′ AAV inverted terminal repeat (3′ ITR); optionally, wherein the sequence encoding the payload is flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to any one of SEQ ID NO: 146-SEQ ID NO: 153; and
selecting for cells expressing the third selectable marker.
123. The method of embodiment 122, further comprising contacting the cell with the triggering agent, wherein in the presence of the triggering agent, the activator activates the inducible promoter resulting in expression of the recombinase, wherein
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein; and recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins.
124. The method of embodiment 123 or 122, wherein a terminal resolution site in the 3′ ITR is deleted.
125. The method of any one of embodiments 122-124, wherein the third polynucleotide construct further comprises a spacer between the 5′ ITR and the sequence encoding the third selectable marker or a spacer between the sequence encoding the third selectable marker and the 3′ ITR, or a combination thereof.
126. The method of embodiment 125, wherein the spacer ranges in length from 500 base pairs to 5000 base pairs.
127. The method of any one of embodiments 122-126, wherein the coding sequence encoding the stop signaling sequence of the first polynucleotide construct further encodes a protein marker.
128. The method of any one of embodiments 122-127, wherein the cell further comprises an adenovirus E1A protein and E1B protein, and the one or more AAV helper proteins expressed by the second polynucleotide construct are an adenovirus E2A protein and E4 protein.
129. The method of any one of embodiments 122-128, wherein the cell further comprises an adenovirus E2A protein and E4 protein, and the one or more AAV helper proteins expressed by the second polynucleotide construct are an adenovirus E1A protein and E1B protein.
130. The method of any one of embodiments 122-129, further comprising a fourth polynucleotide construct comprising an inducible or constitutive promoter operably linked to a sequence encoding one or more helper proteins.
131. The method of embodiment 130, wherein the one or more AAV helper proteins expressed by the second polynucleotide construct are an adenovirus E2A protein and E4 protein and the one or more AAV helper proteins expressed by the fourth polynucleotide construct are an adenovirus E1A protein and E1B protein.
132. The method of embodiment 130, wherein the one or more AAV helper proteins expressed by the second polynucleotide construct are an adenovirus E1A protein and E1B protein and the one or more AAV helper proteins expressed by the fourth polynucleotide construct are an adenovirus E2A protein and E4 protein.
133. The method of any one of embodiments 122-132, wherein the sequence coding for one or more AAV helper proteins comprises a bicistronic open reading frame encoding two AAV helper proteins.
134. The method of embodiment 133, wherein the two AAV helper proteins are E2A and E4 or E1A and E1B.
135. The method of embodiment 133 or 134, wherein the bicistronic open reading frame comprises an internal ribosome entry site (IRES) or a peptide 2A (P2A) sequence.
136. The method of any one of embodiments 122-135, wherein transcription of the AAV Rep coding sequences and the sequence encoding one or more AAV capsid proteins are driven by native AAV promoters.
137. The method of embodiment 136, wherein transcription of the AAV Rep coding sequences is driven by the P5 and P19 native AAV promoters, and transcription of the sequence encoding the one or more AAV capsid proteins is driven by the P40 native AAV promoter.
138. The method of any one of embodiments 122-135, wherein transcription of the AAV Rep coding sequences is driven by an inducible promoter.
139. The method of any one of embodiments 122-138, wherein transcription of the sequence encoding the one or more AAV capsid proteins is driven by an inducible promoter.
140. The method of any one of embodiments 122-139, wherein the AAV capsid proteins comprise VP1, VP2, and VP3.
141. The method of embodiment 140, wherein the third sequence of the first polynucleotide construct further comprises a first inducible promoter operably linked to the sequence encoding the VP1 capsid protein, a second inducible promoter operably linked to the sequence encoding the VP2 capsid protein, or a third inducible promoter operably linked to the sequence encoding the VP3 capsid protein, or a combination thereof.
142. The method of embodiment 141, wherein the third sequence of the first polynucleotide construct further comprises a first inducible promoter operably linked to the sequence encoding the VP1 capsid protein, a second inducible promoter operably linked to the sequence encoding the VP2 capsid protein, and a third inducible promoter operably linked to the sequence encoding the VP3 capsid protein.
143. The method of any one of embodiments 122-142, wherein the cell is a mammalian cell.
144. The method of embodiment 143, wherein the mammalian cell is a HEK293 cell.
145. The method of embodiment 144, wherein the HEK293 cell is DHFR-deficient or GS-deficient.
146. The method of any one of embodiments 122-145, wherein the first polynucleotide construct, the second polynucleotide construct, or the combination thereof are integrated into the nuclear genome of the cell.
147. The method of embodiment 145, wherein the first polynucleotide construct, the second polynucleotide construct, or a combination thereof are integrated into the nuclear genome of the cell using a transposon system, a clustered regularly interspersed short palindromic repeats (CRISPR) system, or a site-specific recombinase.
148. The method of embodiment 147 wherein the transposon system is a Tol2, piggyBac, or Sleeping Beauty transposon system.
149. The method of embodiment 146, wherein the first polynucleotide construct, the second polynucleotide construct, or the combination thereof are provided by a lentivirus vector that integrates into the nuclear genome of the cell.
150. The method of any one of embodiments 122-145, wherein the first polynucleotide construct, the second polynucleotide construct, or the combination thereof are not integrated into the nuclear genome of the cell.
151. The method of embodiment 150, wherein the first polynucleotide construct and the second polynucleotide construct further comprise Epstein-Barr virus (EBV) sequences to stably maintain the constructs extrachromosomally.
152. The method of any one of embodiments 122-151, wherein the inducible promoter in the second polynucleotide construct is selected from the group consisting of a tetracycline-inducible promoter, an ecdysone-inducible promoter, and a cumate-inducible promoter, 153. The method of embodiment 152, wherein the tetracycline-inducible promoter comprises a tetracycline-responsive promoter element (TRE).
154. The method of embodiment 153, wherein the TRE comprises Tet operator (tetO) sequence concatemers fused to a minimal promoter.
155. The method of embodiment 154, wherein the minimal promoter is a human cytomegalovirus promoter or comprises the sequence as set forth in one of SEQ ID NOs: 63-68.
156. The method of any one of embodiments 153-155, wherein the TRE comprises the sequence as set forth in one of SEQ ID NOs: 22, 46-48, and 50-62.
157. The method of any one of embodiments 152-156, wherein the activator comprises the sequence as set forth in one of SEQ ID NOs: 21 and 40 or variants thereof-45.
158. The method of any one of embodiments 152-157, wherein the triggering agent for inducing the tetracycline-inducible promoter is tetracycline or doxycycline.
159. The method of embodiment 158, wherein the activator is a reverse tetracycline-controlled transactivator (rTA) comprising a Tet Repressor binding protein (TetR) fused to a VP16 transactivation domain, and the triggering agent is tetracycline or doxycycline.
160. The method of embodiment 152, wherein the triggering agent for inducing the ecdysone-inducible promoter is ecdysone or ponasterone.
161. The method of embodiment 152, wherein the triggering agent for inducing the cumate-inducible promoter is cumate.
162. The method of embodiment 161, wherein the cumate-inducible promoter comprises a cumate operator sequence.
163. The method of any one of embodiments 122-162, wherein the inducible recombinase is fused to an estrogen response element (ER) and translocates to the nucleus in the presence of tamoxifen.
164. The method of any one of embodiments 122-163, wherein the first selectable marker encoded by the first polynucleotide construct comprises:
a C-terminal fragment of the mammalian DHFR (Cter-DHFR) fused to a leucine zipper peptide, and the selectable marker encoded by the third polynucleotide construct comprises an N-terminal fragment of the mammalian DHFR (Nter-DHFR) fused to a leucine zipper peptide, or an N-terminal fragment of the mammalian DHFR (Nter-DHFR) fused to a leucine zipper peptide, and the selectable marker encoded by the third polynucleotide construct comprises a C-terminal fragment of the mammalian DHFR (Cter-DHFR) fused to a leucine zipper peptide.
165. The method of any one of embodiments 122-163, wherein the selectable marker encoded by the first polynucleotide construct, second polynucleotide construct, or third polynucleotide construct is an auxotrophic protein or antibiotic resistance protein; optionally, wherein the selectable marker is an N-terminal fragment of the auxotrophic protein or antibiotic resistance protein fused to a N-terminal fragment of a split intein; optionally, wherein the an N-terminal fragment of the auxotrophic protein or antibiotic resistance protein fused to a N-terminal fragment of a split intein is any one of SEQ ID NO: 91, 93, 95, 97, 113, 115, 117, 119, 121, 124, 126, 128, 130, or 137; optionally, wherein the selectable marker is a C-terminal fragment of a split intein fused to an C-terminal fragment of the auxotrophic protein or antibiotic resistance protein; optionally, wherein the C-terminal fragment of a split intein fused to an C-terminal fragment of the auxotrophic protein or antibiotic resistance protein comprises any one of SEQ ID NO: 92, 93, 94, 96, 98, 114, 116, 118, 120, 122, 125, 127, 129, 131, or 141.
166. The method of embodiment 165, wherein the selectable marker encoded by the first polynucleotide construct comprises a first antibiotic resistance protein, the selectable marker encoded by the second polynucleotide construct comprises a second antibiotic resistance protein, and the selectable marker encoded by the third polynucleotide construct comprises a third antibiotic resistance protein, and wherein the first antibiotic resistance protein, the second antibiotic resistance protein, and the third antibiotic resistance protein are different.
167. The method of any one of embodiments 122-166, wherein the recombination sites in the first polynucleotide construct and the second polynucleotide construct are lox sites and the recombinase is a cre recombinase or wherein the recombination sites in the first polynucleotide construct and the second polynucleotide are flippase recognition target (FRT) sites and the recombinase is a flippase (Flp) recombinase.
168. The method of any one of embodiments 122-167, wherein upon expression of the inducible recombinase,
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein; and
recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins.
169. The method of any one of embodiments 122-168, wherein the sequence encoding the payload comprises a reporter gene, a therapeutic gene, or a transgene encoding a protein of interest; optionally, wherein the sequence encoding the payload a sequence encoding progranulin.
170. The method of any one of embodiments 122-168, wherein the sequence encoding the payload comprises a suppressor tRNA, a guide RNA or a homology region for homology-directed repair.
171. The method of any one of embodiments 122-170, wherein the second polynucleotide construct further comprises an insert comprising a sequence encoding VA-RNA; or optionally, wherein a fifth construct comprises an insert comprising a sequence encoding VA-RNA.
172. The method of embodiment 171, wherein the VA-RNA is wild-type VA-RNA or VA-RNA comprising one or more mutations in the VA-RNA internal promoter.
173. The method of embodiment 170 or 172, wherein the insert comprises:
a first part of a second constitutive promoter and a second part of a second constitutive promoter separated by a second excisable element comprising a fifth recombination site and a sixth recombination site flanking a stuffer sequence, wherein the fifth and sixth recombination sites are oriented in the same direction, and
a VA-RNA coding sequence, wherein excision of the second excisable element by the inducible recombinase generates a functional complete second constitutive promoter operably linked to the VA-RNA coding sequence to allow expression of the VA-RNA.
174. The method of embodiment 173, wherein the first part of the second constitutive promoter comprises a distal sequence element (DSE) of an RNA polymerase III promoter, and the second part of the second constitutive promoter comprises a proximal sequence element (PSE) of an RNA polymerase III promoter.
175. The method of embodiment 174, wherein the first part of the second constitutive promoter comprises a distal sequence element (DSE) of a U6 promoter, and the second part of the second constitutive promoter comprises a proximal sequence element (PSE) of a U6 promoter.
176. The method of embodiment 174, wherein the first part of the second constitutive promoter comprises a distal sequence element (DSE) of a U7 promoter, and the second part of the second constitutive promoter comprises a proximal sequence element (PSE) of a U7 promoter.
177. The method of any one of embodiments 172-176, wherein the VA-RNA comprises a G16A mutation or a G60A mutation, or a combination thereof.
178. The method of any one of embodiments 122-177, wherein the first polynucleotide construct further comprises:
(i) a first spacer segment and a second spacer segment flanking the excisable element, wherein the first part of the AAV Rep coding sequence and the second part of the AAV Rep coding sequence are separated by the first spacer segment, the excisable element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site; or.
(ii) a first spacer segment and a second spacer segment flanking the inversible element, wherein the first part of the AAV Rep coding sequence and the second part of the AAV Rep coding sequence are separated by the first spacer segment, the inversible element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site.
179. The method of embodiment 178, wherein first part of the AAV Rep coding sequence comprises a p5 internal promoter and a p19 internal promoter, and the second part of the AAV Rep coding sequence comprises a p40 internal promoter.
180. The method of embodiment 179, wherein the excisable spacer is inserted at an insertion site between the p19 internal promoter and the p40 internal promoter of the AAV Rep coding sequence.
181. The method of embodiment 180, wherein the insertion site is between a CAG and a G, a CAG and an A, an AAG and a G, and an AAG and an A.
182. The method of any one of embodiments 122-181, wherein the inducible recombinase is fused to an estrogen response element (ER) and translocates to the nucleus in the presence of tamoxifen.
183. The method of any one of embodiments 122-182, wherein the complete AAV Rep coding sequence comprises an intron.
184. A method for generating a recombinant adenovirus associated virus (rAAV) virion comprising a sequence encoding a payload of interest, the method comprising contacting the cell according to any one of embodiments 2-62 with the triggering agent,
wherein in the presence of the triggering agent, the activator activates the inducible promoter of the second polynucleotide construct resulting in expression of the recombinase, wherein recombination between the first recombination site and the second recombination site in the first polynucleotide construct by the recombinase results in excision of the excisable element or inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein, and recombination between the third recombination site and the fourth recombination site in the second polynucleotide construct by the recombinase results in excision of the self-excising element comprising the sequence encoding the inducible recombinase, wherein the inducible promoter becomes operably linked to the sequence encoding the one or more AAV helper proteins to allow expression of the one or more AAV helper proteins.
wherein the expression of the one or more AAV helper proteins results in expression of the one or more Rep proteins and the one or more capsid proteins, thereby generating an rAAV virion comprising the sequence encoding the payload of interest.
185. The rAAV virion produced by the method of embodiment 184.
186. The rAAV virion of embodiment 185, wherein a terminal resolution site in the 3′ ITR is deleted such that the rAAV virion comprises self-complementary DNA.
187. The rAAV virion of embodiment 185, wherein the 3′ ITR is a wild-type 3′ ITR such that the rAAV virion comprises single-stranded DNA.
188. A cell for inducibly producing recombinant adenovirus associated virus (rAAV) virions, the cell comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction; or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence or the third sequence comprises one or more promoters operably linked to the sequence encoding the one or more AAV capsid proteins; and a first constitutive promoter operably linked to a sequence encoding a first selectable marker; and
a second polynucleotide construct comprising a second constitutive promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding one or more AAV helper proteins, wherein the second constitutive promoter is not operably linked to the sequence encoding the one or more AAV helper proteins; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the one or more AAV helper proteins.
189. A cell for inducibly producing recombinant adenovirus associated virus (rAAV) virions, the cell comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence; and a first constitutive promoter operably linked to a sequence encoding a first selectable marker; and
a second polynucleotide construct comprising a first inducible promoter operably linked to an inducible recombinase; a second inducible promoter operably linked to a sequence encoding one or more AAV helper proteins; a second constitutive promoter operably linked to a sequence encoding an activator, wherein the cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
wherein in absence of activation of the first and second inducible promoters, the cell expresses a fusion protein comprising a Rep protein encoded by the first part of the AAV Rep coding sequence that terminates at the stop signaling sequence, and does not express detectable levels of the inducible recombinase and the one or more AAV helper proteins.
190. A cell for inducibly producing recombinant adenovirus associated virus (rAAV) virions, the cell comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence; and a first constitutive promoter operably linked to a sequence encoding a first selectable marker; and
a second polynucleotide construct comprising a first inducible promoter operably linked to an inducible recombinase; a second inducible promoter operably linked to a sequence encoding an E2A AAV helper protein; a third inducible promoter operably linked to a sequence encoding an E4 AAV helper protein; a second constitutive promoter operably linked to a sequence encoding an activator, wherein the cell constitutively expresses the activator and the activator is unable to activate the inducible promoters in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E2A and E4 AAV helper proteins.
191. The cell of embodiment 190, wherein the second inducible promoter and the third inducible promoter have the same or different orientations.
192. A cell for inducibly producing recombinant adenovirus associated virus (rAAV) virions, the cell comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence; and a first constitutive promoter operably linked to a sequence encoding a first selectable marker; and
a second polynucleotide construct comprising an inducible promoter operably linked to an inducible recombinase; a bidirectional inducible promoter operably linked to a sequence encoding an E2A AAV helper protein and a sequence encoding an E4 AAV helper protein; a second constitutive promoter operably linked to a sequence encoding an activator, wherein the cell constitutively expresses the activator and the activator is unable to activate the inducible promoters in absence of a triggering agent; and a third constitutive promoter operably linked to a sequence encoding a second selectable marker, wherein the cell constitutively expresses the second selectable marker; wherein in absence of activation of the inducible promoter, the cell expresses a fusion protein comprising a Rep protein encoded by the first part of the AAV Rep coding sequence that terminates at the stop signaling sequence; and
wherein in absence of activation of the bidirectional inducible promoter, the cell does not express detectable levels of the inducible recombinase and the E2A and E4 AAV helper proteins.
193. A cell for inducibly producing recombinant adenovirus associated virus (rAAV) virions, the cell comprising:
a polynucleotide construct comprising an inducible promoter operably linked to a self-excising element; the self-excising element comprising a first recombination site and a second recombination site flanking a sequence encoding an inducible recombinase, wherein the first recombination site and the second recombination site are oriented in the same direction; a sequence encoding an E2A AAV helper protein, wherein the inducible promoter is not operably linked to the sequence encoding the E2A AAV helper protein; a first constitutive promoter operably linked to a sequence encoding an activator, wherein the cell constitutively expresses the activator and the activator is unable to activate the inducible promoter in absence of a triggering agent; and a second constitutive promoter operably linked to a sequence encoding a selectable marker, wherein the cell constitutively expresses the selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E2A AAV helper protein.
194. A cell for inducibly producing recombinant adenovirus associated virus (rAAV) virions, the cell comprising:
a polynucleotide construct comprising an inducible promoter operably linked to a self-excising element; the self-excising element comprising a first recombination site and a second recombination site flanking a sequence encoding an inducible recombinase, wherein the first recombination site and the second recombination site are oriented in the same direction; a sequence encoding an E4 AAV helper protein, wherein the inducible promoter is not operably linked to the sequence encoding the E4 AAV helper protein; a first constitutive promoter operably linked to a sequence encoding an activator, wherein the cell constitutively expresses the activator and the activator is unable to activate the inducible promoter in absence of a triggering agent; and a second constitutive promoter operably linked to a sequence encoding a selectable marker, wherein the cell constitutively expresses the selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E4 AAV helper protein.
195. A cell for inducibly producing recombinant adenovirus associated virus (rAAV) virions, the cell comprising:
a polynucleotide construct comprising an inducible promoter operably linked to a self-excising element; the self-excising element comprising a first recombination site and a second recombination site flanking a sequence encoding an inducible recombinase, wherein the first recombination site and the second recombination site are oriented in the same direction; a sequence encoding an E1A AAV helper protein, wherein the inducible promoter is not operably linked to the sequence encoding the E1A AAV helper protein; a first constitutive promoter operably linked to a sequence encoding an activator, wherein the cell constitutively expresses the activator and the activator is unable to activate the inducible promoter in absence of a triggering agent; and a second constitutive promoter operably linked to a sequence encoding a selectable marker, wherein the cell constitutively expresses the selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E1A AAV helper protein.
196. A cell for inducibly producing recombinant adenovirus associated virus (rAAV) virions, the cell comprising:
a polynucleotide construct comprising an inducible promoter operably linked to a self-excising element; the self-excising element comprising a first recombination site and a second recombination site flanking a sequence encoding an inducible recombinase, wherein the first recombination site and the second recombination site are oriented in the same direction; a sequence encoding an E1B AAV helper protein, wherein the inducible promoter is not operably linked to the sequence encoding the E1B AAV helper protein; a first constitutive promoter operably linked to a sequence encoding an activator, wherein the cell constitutively expresses the activator and the activator is unable to activate the inducible promoter in absence of a triggering agent; and a second constitutive promoter operably linked to a sequence encoding a selectable marker, wherein the cell constitutively expresses the selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E1B AAV helper protein.
197. A cell for inducibly producing recombinant adenovirus associated virus (rAAV) virions, the cell comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding a VP1 AAV capsid protein, wherein the second sequence comprises a promoter that is operably linked to the third sequence; and a first constitutive promoter operably linked to a sequence encoding a first selectable marker.
198. A cell for inducibly producing recombinant adenovirus associated virus (rAAV) virions, the cell comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding a VP2 AAV capsid protein, wherein the second sequence comprises a promoter that is operably linked to the third sequence; and a first constitutive promoter operably linked to a sequence encoding a first selectable marker.
199. A cell for inducibly producing recombinant adenovirus associated virus (rAAV) virions, the cell comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding a VP3 AAV capsid protein, wherein the second sequence comprises a promoter that is operably linked to the third sequence; and a first constitutive promoter operably linked to a sequence encoding a first selectable marker.
200. A vector system for inducibly producing recombinant adenovirus associated virus (rAAV) virions, the vector system comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding a VP1 AAV capsid protein, wherein the second sequence comprises a promoter that is operably linked to the third sequence; and a first constitutive promoter operably linked to a sequence encoding a first selectable marker; a second polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by an excisable element that comprises a first recombination site and a second recombination site flanking the coding sequence encoding the stop signaling sequence, wherein the first recombination site and the second recombination site are oriented in the same direction, and wherein the one or more promoters are not operably linked to the second sequence comprising the second part of the AAV Rep coding sequence; a third sequence comprising a sequence encoding a VP2 AAV capsid protein, wherein the second sequence comprises a promoter that is operably linked to the third sequence; and a first constitutive promoter operably linked to a sequence encoding a first selectable marker; and
a third polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by an excisable element that comprises a first recombination site and a second recombination site flanking the coding sequence encoding the stop signaling sequence, wherein the first recombination site and the second recombination site are oriented in the same direction, and wherein the one or more promoters are not operably linked to the second sequence comprising the second part of the AAV Rep coding sequence; a third sequence comprising a sequence encoding a VP3 AAV capsid protein, wherein the second sequence comprises a promoter that is operably linked to the third sequence; and a first constitutive promoter operably linked to a sequence encoding a first selectable marker.
201. The vector system of embodiment 200, further comprising a polynucleotide construct comprising an inducible promoter operably linked to an inducible recombinase; a bidirectional inducible promoter operably linked to a sequence encoding an E2A AAV helper protein and a sequence encoding an E4 AAV helper protein; a second constitutive promoter operably linked to a sequence encoding an activator, wherein the cell constitutively expresses the activator and the activator is unable to activate the inducible promoters in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence; and
wherein in absence of activation of the bidirectional inducible promoter, the cell does not express detectable levels of the inducible recombinase and the E2A and E4 AAV helper proteins.
202. The vector system of embodiment 200, further comprising a polynucleotide construct comprising a first inducible promoter operably linked to an inducible recombinase; a second inducible promoter operably linked to a sequence encoding an E2A AAV helper protein; a third inducible promoter operably linked to a sequence encoding an E4 AAV helper protein; a second constitutive promoter operably linked to a sequence encoding an activator, wherein the cell constitutively expresses the activator and the activator is unable to activate the inducible promoters in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E2A and E4 AAV helper proteins.
203. The vector system of embodiment 202, wherein the second inducible promoter and the third inducible promoter have the same or different orientations.
204. The vector system of embodiment 200, further comprising a polynucleotide construct comprising an inducible promoter operably linked to an inducible recombinase; a bidirectional inducible promoter operably linked to a sequence encoding an E2A AAV helper protein and a sequence encoding an E4 AAV helper protein; a second constitutive promoter operably linked to a sequence encoding an activator, wherein the cell constitutively expresses the activator and the activator is unable to activate the inducible promoters in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence; and
wherein in absence of activation of the bidirectional inducible promoter, the cell does not express detectable levels of the inducible recombinase and the E2A and E4 AAV helper proteins.
205. The vector system of embodiment 200, further comprising a polynucleotide construct comprising an inducible promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding an E2A AAV helper protein, wherein the inducible promoter is not operably linked to the sequence encoding the E2A AAV helper protein; a second constitutive promoter operably linked to a sequence encoding an activator, wherein the cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E2A AAV helper protein.
206. The vector system of embodiment 200 or 205, further comprising a polynucleotide construct comprising a second polynucleotide construct comprising an inducible promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding an E4 AAV helper protein, wherein the inducible promoter is not operably linked to the sequence encoding the E4 AAV helper protein; a second constitutive promoter operably linked to a sequence encoding an activator, wherein the cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E4 AAV helper protein.
207. The vector system of embodiment 200, 205, or 206, further comprising a polynucleotide construct comprising an inducible promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding an EA AAV helper protein, wherein the inducible promoter is not operably linked to the sequence encoding the E1A AAV helper protein; a second constitutive promoter operably linked to a sequence encoding an activator, wherein the cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E1A AAV helper protein.
208. The vector system of embodiment 100, further comprising a polynucleotide construct comprising an inducible promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding an E1B AAV helper protein, wherein the inducible promoter is not operably linked to the sequence encoding the E1B AAV helper protein; a second constitutive promoter operably linked to a sequence encoding an activator, wherein the cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the E1B AAV helper protein.
209. A vector system for inducibly producing recombinant adenovirus associated virus (rAAV) virions, the vector system comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence; and a first constitutive promoter operably linked to a sequence encoding a first selectable marker; and
a second polynucleotide construct comprising a first inducible promoter operably linked a sequence encoding an inducible recombinase; a second inducible promoter operably linked to a sequence encoding one or more AAV helper proteins; a second constitutive promoter operably linked to a sequence encoding an activator, wherein a cell constitutively expresses the activator and the activator is unable to activate the inducible promoter in absence of a triggering agent; and a third constitutive promoter operably linked to a sequence encoding a second selectable marker, wherein the cell constitutively expresses the second selectable marker;
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the one or more AAV helper proteins.
210. A vector system for inducibly producing recombinant adenovirus associated virus (rAAV) virions, the vector system comprising:
a first polynucleotide construct comprising one or more promoters operably linked to a first sequence comprising a first part of an AAV Rep coding sequence and a coding sequence encoding a stop signaling sequence; a second sequence comprising a second part of the AAV Rep coding sequence, wherein the first sequence comprising the first part of the AAV Rep coding sequence and the second sequence comprising the second part of the AAV Rep coding sequence are separated by (i) an excisable element that comprises a first recombination site and a second recombination site flanking the coding sequence encoding the stop signaling sequence, wherein the first recombination site and the second recombination site are oriented in the same direction or (ii) an inversible element that comprises a first recombination site and a second recombination site flanking the coding sequence encoding the stop signaling sequence, wherein the first recombination site and the second recombination site are oriented in opposite directions, and wherein the one or more promoters are not operably linked to the second sequence comprising the second part of the AAV Rep coding sequence; a third sequence comprising a sequence encoding one or more AAV capsid proteins, wherein the second sequence comprises a promoter that is operably linked to the third sequence; and a first constitutive promoter operably linked to a sequence encoding a first selectable marker; and
a second polynucleotide construct comprising a second constitutive promoter operably linked to a self-excising element; the self-excising element comprising a third recombination site and a fourth recombination site flanking a sequence encoding an inducible recombinase (e.g., Cre-ERT2), wherein the third recombination site and the fourth recombination site are oriented in the same direction; a sequence encoding one or more AAV helper proteins, wherein the second constitutive promoter is not operably linked to the sequence encoding the one or more AAV helper proteins; and a third constitutive promoter operably linked to a sequence encoding a second selectable marker, wherein the cell constitutively expresses the second selectable marker;
wherein in absence of activation of the inducible recombinase, the cell expresses a fusion protein comprising a Rep protein encoded by the first part of the AAV Rep coding sequence that terminates at the stop signaling sequence, and does not express detectable levels of the inducible recombinase and the one or more AAV helper proteins.
211. The vector system of embodiment 210, further comprising a third polynucleotide construct comprising a promoter operably linked to a sequence encoding a payload and a third selectable marker, wherein the sequence encoding the payload is flanked by a 5′ AAV inverted terminal repeat (5′ ITR) and a 3′ AAV inverted terminal repeat (3′ ITR); optionally, wherein the sequence encoding the payload is flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to SEQ ID NO: 147.
212. The vector system of embodiments 210 or 211, wherein a terminal resolution site in the 3′ ITR is deleted.
213. The vector system of embodiment 211 or 212, wherein the third polynucleotide construct wherein the third polynucleotide construct further comprises a spacer between the 5′ ITR and the sequence encoding the third selectable marker or a spacer between the sequence encoding the third selectable marker and the 3′ ITR, or a combination thereof.
214. The vector system of embodiment 213, wherein the spacer ranges in length from 500 base pairs to 5000 base pairs.
215. The vector system of any one of embodiments 210-214, wherein the coding sequence encoding the stop signaling sequence of the first polynucleotide construct further encodes a protein marker that comprises the stop signaling sequence.
216. The vector system of any one of embodiments 210-215, wherein the one or more AAV helper proteins expressed by the second polynucleotide construct are an adenovirus E2A protein and E4 protein.
217. The vector system of any one of embodiments 210-216, wherein the one or more AAV helper proteins expressed by the second polynucleotide construct are an adenovirus E1A protein and E1B protein.
218. The vector system of any one of embodiments 210-217, further comprising a fourth polynucleotide construct comprising an inducible or constitutive promoter operably linked to a sequence encoding one or more helper proteins.
219. The vector system of embodiment 218, wherein the one or more AAV helper proteins expressed by the second polynucleotide construct are an adenovirus E2A protein and E4 protein and the one or more AAV helper proteins expressed by the fourth polynucleotide construct are an adenovirus E1A protein and E1B protein.
220. The vector system of embodiment 218, wherein the one or more AAV helper proteins expressed by the second polynucleotide construct are an adenovirus E1A protein and E1B protein and the one or more AAV helper proteins expressed by the fourth polynucleotide construct are an adenovirus E2A protein and E4 protein.
221. The vector system of any one of embodiments 210-220, wherein the sequence coding for one or more AAV helper proteins comprises a bicistronic open reading frame encoding two AAV helper proteins.
222. The vector system of embodiment 221, wherein the two AAV helper proteins are E2A and E4 or E1A and E1B.
223. The vector system of embodiment 221 or 222, wherein the bicistronic open reading frame comprises an internal ribosome entry site (IRES) or a peptide 2A (P2A) sequence.
224. The vector system of any one of embodiments 210-223, wherein transcription of the AAV Rep coding sequences and the sequence encoding one or more AAV capsid proteins are driven by native AAV promoters.
225. The vector system of embodiment 224, wherein transcription of the AAV Rep coding sequences is driven by the P5 and P19 native AAV promoters, and transcription of the sequence encoding the one or more AAV capsid proteins is driven by the P40 native AAV promoter.
226. The vector system of any one of embodiments 210-225, wherein the AAV capsid proteins comprise VP1, VP2, and VP3.
227. The vector system of any one of embodiments 210-226, wherein the first polynucleotide construct, the second polynucleotide construct, or the combination thereof are integrated into the nuclear genome of a cell.
228. The vector system of embodiment 227, wherein the first polynucleotide construct, the second polynucleotide construct, or a combination thereof are integrated into the nuclear genome of the cell using a transposon system, a clustered regularly interspersed short palindromic repeats (CRISPR) system, or a site-specific recombinase.
229. The vector system of embodiment 228 wherein the transposon system is a Tol2, piggyBac, or Sleeping Beauty transposon system.
230. The vector system of embodiment 228, wherein the first polynucleotide construct, the second polynucleotide construct, or the combination thereof are provided by a lentivirus vector that integrates into the nuclear genome of a cell.
231. The vector system of any one of embodiments 210-230, wherein the first polynucleotide construct, the second polynucleotide construct, or the combination thereof are not integrated into the nuclear genome of a cell.
232. The vector system of embodiment 231, wherein the first polynucleotide construct and the second polynucleotide construct further comprise Epstein-Barr virus (EBV) sequences to stably maintain the constructs extrachromosomally.
233. The vector system of any one of embodiments 210-232, wherein the inducible recombinase is fused to an estrogen response element (ER) and translocates to the nucleus in the presence of tamoxifen.
234. The vector system of any one of embodiments 210-233, wherein the first selectable marker encoded by the first polynucleotide construct comprises:
a C-terminal fragment of the mammalian DHFR (Cter-DHFR) fused to a leucine zipper peptide, and the selectable marker encoded by the third polynucleotide construct comprises an N-terminal fragment of the mammalian DHFR (Nter-DHFR) fused to a leucine zipper peptide, or an N-terminal fragment of the mammalian DHFR (Nter-DHFR) fused to a leucine zipper peptide, and the selectable marker encoded by the third polynucleotide construct comprises a C-terminal fragment of the mammalian DHFR (Cter-DHFR) fused to a leucine zipper peptide.
235. The vector system of any one of embodiments 210-233, wherein the selectable marker encoded by the first polynucleotide construct, second polynucleotide construct, or third polynucleotide construct is an auxotrophic protein or antibiotic resistance protein; optionally, wherein the selectable marker is an N-terminal fragment of the auxotrophic protein or antibiotic resistance protein fused to a N-terminal fragment of a split intein; optionally, wherein the an N-terminal fragment of the auxotrophic protein or antibiotic resistance protein fused to a N-terminal fragment of a split intein is any one of SEQ ID NO: 91, 93, 95, 97, 113, 115, 117, 119, 121, 124, 126, 128, 130, or 137; optionally, wherein the selectable marker is a C-terminal fragment of a split intein fused to an C-terminal fragment of the auxotrophic protein or antibiotic resistance protein; optionally, wherein the C-terminal fragment of a split intein fused to an C-terminal fragment of the auxotrophic protein or antibiotic resistance protein comprises any one of SEQ ID NO: 92, 93, 94, 96, 98, 114, 116, 118, 120, 122, 125, 127, 129, 131, or 141
236. The vector system of embodiment 235, wherein the selectable marker encoded by the first polynucleotide construct comprises a first antibiotic resistance protein, the selectable marker encoded by the second polynucleotide construct comprises a second antibiotic resistance protein, and the selectable marker encoded by the third polynucleotide construct comprises a third antibiotic resistance protein, and wherein the first antibiotic resistance protein, the second antibiotic resistance protein, and the third antibiotic resistance protein are different.
237. The vector system of any one of embodiments 210-236, wherein the recombination sites in the first polynucleotide construct and the second polynucleotide construct are lox sites and the recombinase is a cre recombinase or wherein the recombination sites in the first polynucleotide construct and the second polynucleotide are flippase recognition target (FRT) sites and the recombinase is a flippase (Flp) recombinase.
238. The vector system of any one of embodiments 210-237, wherein upon expression of the inducible recombinase,
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein; and
recombination between the third recombination site and the fourth recombination site in the second polynucleotide construct results in excision of the self-excising element comprising the sequence encoding the inducible recombinase, wherein the second constitutive promoter becomes operably linked to the sequence encoding the one or more AAV helper proteins to allow expression of the one or more AAV helper proteins.
239. The vector system of any one of embodiments 211-238, wherein the sequence encoding the payload comprises a reporter gene, a therapeutic gene, or a transgene encoding a protein of interest; optionally, wherein the sequence encoding the payload comprises a sequence encoding progranulin.
240. The vector system of any one of embodiments 211-239, wherein the sequence encoding the payload comprises a suppressor tRNA, a guide RNA or a homology region for homology-directed repair.
241. The vector system of any one of embodiments 210-240, wherein the second polynucleotide construct further comprises an insert comprising VA-RNA or a fourth construct comprises an insert comprising VA-RNA.
242. The vector system of embodiment 241, wherein the VA-RNA is wild-type VA-RNA or VA-RNA comprising one or more mutations in the VA-RNA internal promoter.
243. The vector system of embodiment 241 or 242, wherein the insert comprises:
a first part of a fourth constitutive promoter and a second part of a fourth constitutive promoter separated by a second excisable element comprising a fifth recombination site and a sixth recombination site flanking a stuffer sequence, wherein the fifth and sixth recombination sites are oriented in the same direction, and
a VA-RNA coding sequence, wherein excision of the second excisable element by the inducible recombinase generates a functional complete second constitutive promoter operably linked to the VA-RNA coding sequence to allow expression of the VA-RNA.
244. The vector system of embodiment 243, wherein the first part of the fourth constitutive promoter comprises a distal sequence element (DSE) of an RNA polymerase III promoter, and the second part of the fourth constitutive promoter comprises a proximal sequence element (PSE) of an RNA polymerase III promoter.
245. The vector system of embodiment 243, wherein the first part of the fourth constitutive promoter comprises a distal sequence element (DSE) of a U6 promoter, and the second part of the fourth constitutive promoter comprises a proximal sequence element (PSE) of a U6 promoter.
246. The vector system of embodiment 243, wherein the first part of the fourth constitutive promoter comprises a distal sequence element (DSE) of a U7 promoter, and the second part of the fourth constitutive promoter comprises a proximal sequence element (PSE) of a U7 promoter.
247. The vector system of any one of embodiments 241-246, wherein the VA-RNA comprises a G16A mutation or a G60A mutation, or a combination thereof.
248. The vector system of any one of embodiments 210-247, wherein the first polynucleotide construct further comprises:
(i) a first spacer segment and a second spacer segment flanking the excisable element, wherein the first part of the AAV Rep coding sequence and the second part of the AAV Rep coding sequence are separated by the first spacer segment, the excisable element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site; or
(ii) a first spacer segment and a second spacer segment flanking the inversible element, wherein the first part of the AAV Rep coding sequence and the second part of the AAV Rep coding sequence are separated by the first spacer segment, the inversible element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site.
249. The vector system of embodiment 248, wherein first part of the AAV Rep coding sequence comprises a p5 internal promoter and a p19 internal promoter, and the second part of the AAV Rep coding sequence comprises a p40 internal promoter.
250. The vector system of embodiment 249, wherein the excisable spacer is inserted at an insertion site between the p19 internal promoter and the p40 internal promoter of the AAV Rep coding sequence.
251. The vector system of embodiment 250, wherein the insertion site is between a CAG and a G, a CAG and an A, an AAG and a G, and an AAG and an A.
252. The vector system of any one of embodiments 210-251, wherein the inducible recombinase is fused to an estrogen response element (ER) and translocates to the nucleus in the presence of tamoxifen.
253. The vector system of any one of embodiments 210-252, wherein the complete AAV Rep coding sequence comprises an intron.
254. A method for increasing production of rAAV virions from a cell, the method comprising: amplifying expression of AAV Rep and capsid proteins, helper proteins, and/or payload in the cell, wherein the amplifying comprises:
increasing copy number of a polynucleotide construct comprising a sequence encoding one or more AAV Rep proteins and a sequence encoding one or more AAV cap proteins, a polynucleotide construct comprising a sequence encoding one or more AAV helper proteins, and/or a polynucleotide construct comprising a sequence encoding the payload;
introducing an agent to amplify expression of the Rep/Cap genes, helper genes, and/or payload.
255. The method of embodiment 254, wherein the polynucleotide construct further comprises a selectable marker operably linked to an attenuated promoter.
256. The method of embodiment 255, wherein the increasing copy number of the polynucleotide construct comprises culturing the cell under conditions that select for the presence of the selectable marker, thereby producing the cell comprising an increased copy number of the polynucleotide construct compared to the polynucleotide construct further comprising a selectable marker operably linked to a nonattenuated promoter.
257. The method of embodiments 254 or 255, wherein the attenuated promoter is an attenuated EF1alpha promoter and the nonattenuated promoter is an EF1alpha promoter; optionally, wherein the attenuated EF1alpha promoter is SEQ ID NO: 132 and the EF1alpha promoter is SEQ ID NO: 133.
258. The method of embodiment 254, wherein the polynucleotide construct further comprises a mutated selectable marker having decreased enzymatic activity compared to an unmutated selectable marker.
259. The method of embodiment 258, wherein the increasing copy number of the polynucleotide construct comprises culturing the cell under conditions that select for the presence of the mutated selectable marker, thereby producing the cell comprising an increased copy number of the polynucleotide construct compared to the polynucleotide construct further comprising the unmutated selectable marker.
260. The method of embodiments 258 or 259, wherein the mutated selectable marker is a mutated GS and the unmutated selectable marker is GS; optionally, wherein the mutated GS having a R324C, R324S, or R341C mutation as compared to SEQ ID NO: 112 and the GS is SEQ ID NO: 112; optionally, wherein the mutated GS is SEQ ID NO: 142, SEQ ID NO: 143, or SEQ ID NO: 144.
261. The method of embodiment 254, wherein the polynucleotide construct further comprises a selectable marker.
262. The method of embodiment 261, wherein the increasing copy number of the polynucleotide construct comprises culturing the cell under conditions that select for the presence of the selectable marker and in the presence of an inhibitor of the selectable marker, thereby producing the cell comprising an increased copy number of the polynucleotide construct compared to the polynucleotide construct further comprising the selectable marker cultured in the absence of the inhibitor of the selectable marker.

### EXAMPLES

INFORMAL SEQUENCE LISTING

First spacer segment- SEQ ID NO: 1:
GTGAGTTTGGGGACCCTTGATTGTTCTTTCTTTTTCGCTATTGTAAAATTC
ATGTTATATGGAGGGGGCAAAGTTTTCAGGGTGTTGTTTAGAATGGGAAGATGTCCC
TTGTATCACCATGGACCCTCATGATAATTTTGTTTCTTTCACTTTCTACTCTGTTGACA
ACCATTGTCTCCTCTTATTTTCTTTTCATTTTCTGTAACTTTTTCGTTAAACTTTAGCTT
GCATTTGTAACGAATTTTTAAATTCACTTTTGTTTATTTGTCAGATTGTAAGTACTTTC
TCTAATCACTTTTTTTTCAAGGCAATCAGGGTATATTATATTGTACTTCAGCACAGTTT
TAGAGAAC
Second spacer segment - SEQ ID NO: 2:
ataacttcgtataatgtatgctatacgaagttatCGGGCCCCTCTGCTAACCATGTTCATGCCTT
CTTCTTTTTCCTACAGatggtgagcaagggcgaggagctgttcaccggggtggtgcccatcctggtcgagctggacggcgac
gtaaacggccacaagttcagcgtgtccggcgagggcgagggcgatgccacctacggcaagctgaccctgaagttcatctgcaccaccggc
aagctgcccgtgccctggcccaccctcgtgaccaccctgacctacggcgtgcagtgcttcagccgctaccccgaccacatgaagcagcac
gacttcttcaagtccgccatgcccgaaggctacgtccaggagcgcaccatcttcttcaaggacgacggcaactacaagacccgcgccgagg
tgaagttcgagggcgacaccctggtgaaccgcatcgagctgaagggcatcgacttcaaggaggacggcaacatcctggggcacaagctg
gagtacaactacaacagccacaacgtctatatcatggccgacaagcagaagaacggcatcaaggtgaacttcaagatccgccacaacatcg
aggacggcagcgtgcagctcgccgaccactaccagcagaacacccccatcggcgacggccccgtgctgctgcccgacaaccactacctg
agcacccagtccgccctgagcaaagaccccaacgagaagcgcgatcacatggtcctgctggagttcgtgaccgccgccgggatcactctc
ggcatggacgagctgtacaagtaaCCTCAGGTGCAGGCTGCCTATCAGAAGGTGGTGGCTGGTGTG
GCCAATGCCCTGGCTCACAAATACCACTGAGATCTTTTTCCCTCTGCCAAAAATTATG
GGGACATCATGAAGCCCCTTGAGCATCTGACTTCTGGCTAATAAAGGAAATTTATTTT
CATTGCAATAGTGTGTTGGAATTTTTTGTGTCTCTCACTCGGAAGGACATATGGGAGG
GCAAATCATTTAAAACATCAGAATGAGTATTTGGTTTAGAGTTTGGCAACATATGCCC
ATATGCTGGCTGCCATGAACAAAGGTTGGCTATAAAGAGGTCATCAGTATATGAAAC
AGCCCCCTGCTGTCCATTCCTTATTCCATAGAAAAGCCTTGACTTGAGGTTAGATTTTT
TTTATATTTTGTTTTGTGTTATTTTTTTCTTTAACATCCCTAAAATTTTCCTTACATGTT
TTACTAGCCAGATTTTTCCTCCTCTCCTGACTACTCCCAGTCATAGCTGTCCCTCTTCT
CTTATGGAGATCataacttcgtataatgtatgctatacgaagttat
Third spacer segment - SEQ ID NO: 3:
AATTGTTATAATTAAATGATAAGGTAGAATATTTCTGCATATAAATTCTG
GCTGGCGTGGAAATATTCTTATTGGTAGAAACAACTACACCCTGGTCATCATCCTGCC
TTTCTCTTTATGGTTACAATGATATACACTGTTTGAGATGAGGATAAAATACTCTGAG
TCCAAACCGGGCCCCTCTGCTAACCATGTTCATGCCTTCTTCTtTTTCCTACAG
DHFR Z-Nter - SEQ ID NO: 4:
ATGAGAGGGTCAGATCCGGCAGCGTTGAAGCGGGCTAGGAACACTGAGG
CAGCAAGGCGCTCTCGAGCAAGGAAGTTGCAACGGATGAAACAATTGGAAGACAAA
GTTGAAGAGCTGCTTTCAAAAAACTACCACCTTGAAAATGAAGTCGCGAGGCTGAAG
AAATTGGTCGGATCTGCTGGCAGCGCAGCGGGGAGCGGTGAGTTTATGGTCAGACCT
CTCAACTGTATTGTCGCTGTCTCACAGAACATGGGTATCGGAAAGAACGGTGACTTG
CCGTGGCCGCCACTGCGGAATGAGTTCAAATACTTTCAGCGCATGACGACCACCAGC
AGTGTGGAGGGTAAGCAAAATCTTGTCATAATGGGTCGCAAGACTTGGTTTTCTATTC
CAGAGAAAAACAGACCGCTTAAAGATAGGATTAACATCGTGTTGAGCCGGGAACTG
AAAGAGCCACCAAGGGGAGCACATTTTTTGGCTAAGTCCTTGGATGACGCCCTGCGA
CTGATAGAGCAACCAGAACTTGCTTAGTAA
DHFR Z-Cter - SEQ ID NO: 5:
ATGCGCGGTTCCGACCCAGCAGCTTTGAAACGAGCACGAAACACGGAAG
CAGCCCGCAGGAGTCGAGCGAGAAAACTTCAGCGGATGAAGCAGCTTGAAGATAAA
GTCGAGGAATTGCTTAGCAAGAATTATCACCTCGAGAATGAAGTGGCGCGACTGAAA
AAACTTGTAGGTTCTGCTGGGAGCGCAGCCGGAAGCGGCGAGTTCTCAAAAGTTGAC
ATGGTGTGGATCGTGGGTGGAAGTTCTGTCTATCAAGAGGCGATGAATCAGCCTGGC
CACCTCAGACTGTTTGTTACAAGGATCATGCAGGAGTTCGAGTCTGACACGTTTTTTC
CAGAGATCGACCTGGGGAAATATAAACTCCTCCCAGAGTACCCAGGAGTGCTTAGTG
AGGTCCAAGAAGAGAAGGGAATCAAATATAAATTTGAAGTTTACGAAAAGAAGGAT
TAGTAA
ITR deleted AAV2 genome with Construct 1 (cloned in pCRII Topo vector)
SEQ ID NO: 6
ggaggggtggagtcgtgacgtgaattacgtcatagggttagggaggtcctgtattagaggtcacgtgagtgttttgcgaca
ttttgcgacaccatgtggtcacgctgggtatttaagcccgagtgagcacgcagggtctccATTTTGAAGCGGGAGGTTTG
AACGCGCAGCCGCCatgccggggttttacgagattgtgattaaggtccccagcgaccttgacgagcatctgcccggcatttctga
cagctttgtgaactgggtggccgagaaggaatgggagttgccgccagattctgacatggatctgaatctgattgagcaggcacccctgaccg
tggccgagaagctgcagcgcgactttctgacggaatggcgccgtgtgagtaaggccccggaggcccttttctttgtgcaatttgagaaggga
gagagctacttccacatgcacgtgctcgtggaaaccaccggggtgaaatccatggttttgggacgtttcctgagtcagattcgcgaaaaactg
attcagagaatttaccgcgggatcgagccgactttgccaaactggttcgcggtcacaaagaccagaaatggcgccggaggcgggaacaag
gtggtggatgagtgctacatccccaattacttgctccccaaaacccagcctgagctccagtgggcgtggactaatatggaacagtatttaagcg
cctgtttgaatctcacggagcgtaaacggttggtggcgcagcatctgacgcacgtgtcgcagacgcaggagcagaacaaagagaatcaga
atcccaattctgatgcgccggtgatcagatcaaaaacttcagccaggtacatggagctggtcgggtggctcgtggacaaGGTGAGTT
TGGGGACCCTTGATTGTTCTTTCTTTTTCGCTATTGTAAAATTCATGTTATATGGAGGG
GGCAAAGTTTTCAGGGTGTTGTTTAGAATGGGAAGATGTCCCTTGTATCACCATGGAC
CCTCATGATAATTTTGTTTCTTTCACTTTCTACTCTGTTGACAACCATTGTCTCCTCTTA
TTTTCTTTTCATTTTCTGTAACTTTTTCGTTAAACTTTAGCTTGCATTTGTAACGAATTT
TTAAATTCACTTTTGTTTATTTGTCAGATTGTAAGTACTTTCTCTAATCACTTTTTTTTC
AAGGCAATCAGGGTATATTATATTGTACTTCAGCACAGTTTTAGAGAACataacttcgtataat
gtatgctatacgaagttatCGGGCCCCTCTGCTAACCATGTTCATGCCTTCTTCTTTTTCCTACAGat
ggtgagcaagggcgaggagctgttcaccggggtggtgcccatcctggtcgagctggacggcgacgtaaacggccacaagttcagcgtgtc
cggcgagggcgagggcgatgccacctacggcaagctgaccctgaagttcatctgcaccaccggcaagctgcccgtgccctggcccaccc
tcgtgaccaccctgacctacggcgtgcagtgcttcagccgctaccccgaccacatgaagcagcacgacttcttcaagtccgccatgcccgaa
ggctacgtccaggagcgcaccatcttcttcaaggacgacggcaactacaagacccgcgccgaggtgaagttcgagggcgacaccctggtg
aaccgcatcgagctgaagggcatcgacttcaaggaggacggcaacatcctggggcacaagctggagtacaactacaacagccacaacgt
ctatatcatggccgacaagcagaagaacggcatcaaggtgaacttcaagatccgccacaacatcgaggacggcagcgtgcagctcgccga
ccactaccagcagaacacccccatcggcgacggccccgtgctgctgcccgacaaccactacctgagcacccagtccgccctgagcaaag
accccaacgagaagcgcgatcacatggtcctgctggagttcgtgaccgccgccgggatcactctcggcatggacgagctgtacaagtaaC
CTCAGGTGCAGGCTGCCTATCAGAAGGTGGTGGCTGGTGTGGCCAATGCCCTGGCTC
ACAAATACCACTGAGATCTTTTTCCCTCTGCCAAAAATTATGGGGACATCATGAAGCC
CCTTGAGCATCTGACTTCTGGCTAATAAAGGAAATTTATTTTCATTGCAATAGTGTGT
TGGAATTTTTTGTGTCTCTCACTCGGAAGGACATATGGGAGGGCAAATCATTTAAAAC
ATCAGAATGAGTATTTGGTTTAGAGTTTGGCAACATATGCCCATATGCTGGCTGCCAT
GAACAAAGGTTGGCTATAAAGAGGTCATCAGTATATGAAACAGCCCCCTGCTGTCCA
TTCCTTATTCCATAGAAAAGCCTTGACTTGAGGTTAGATTTTTTTTATATTTTGTTTTG
TGTTATTTTTTTCTTTAACATCCCTAAAATTTTCCTTACATGTTTTACTAGCCAGATTTT
TCCTCCTCTCCTGACTACTCCCAGTCATAGCTGTCCCTCTTCTCTTATGGAGATCataactt
cgtataatgtatgctatacgaagttatAATTGTTATAATTAAATGATAAGGTAGAATATTTCTGCATAT
AAATTCTGGCTGGCGTGGAAATATTCTTATTGGTAGAAACAACTACACCCTGGTCATC
ATCCTGCCTTTCTCTTTATGGTTACAATGATATACACTGTTTGAGATGAGGATAAAAT
ACTCTGAGTCCAAACCGGGCCCCTCTGCTAACCATGTTCATGCCTTCTTCTtTTTCCTA
CAGgggattacctcggagaagcagtggatccaggaggaccaggcctcatacatctccttcaatgcggcctccaactcgcggtcccaaatc
aaggctgccttggacaatgcgggaaagattatgagcctgactaaaaccgcccccgactacctggtgggccagcagcccgtggaggacattt
ccagcaatcggatttataaaattttggaactaaacgggtacgatccccaatatgcggcttccgtctttctgggatgggccacgaaaaagttcgg
caagaggaacaccatctggctgtttgggcctgcaactaccgggaagaccaacatcgcggaggccatagcccacactgtgcccttctacggg
tgcgtaaactggaccaatgagaactttcccttcaacgactgtgtcgacaagatggtgatctggtgggaggaggggaagatgaccgccaaggt
cgtggagtcggccaaagccattctcggaggaagcaaggtgcgcgtggaccagaaatgcaagtcctcggcccagatagacccgactcccgt
gatcgtcacctccaacaccaacatgtgcgccgtgattgacgggaactcaacgaccttcgaacaccagcagccgttgcaagaccggatgttca
aatttgaactcacccgccgtctggatcatgactttgggaaggtcaccaagcaggaagtcaaagactttttccggtgggcaaaggatcacgtgg
ttgaggtggagcatgaattctacgtcaaaaagggtggagccaagaaaagacccgcccccagtgacgcagatataagtgagcccaaacggg
tgcgcgagtcAGTTGCGCagccatcgacgtcagacgcggaagcttcgatcaactacgcagacagGTACCAAAACAAA
TGTTCTCGTCACGTGGGCATGAATCTGATGCTGTTTCCCTGCAGACAATGCGAGAGAA
TGAATCAGAATTCAAATATCTGCTTCACTCACGGACAGAAAGACTGTTTAGAGTGCTT
TCCCGTGTCAGAATCTCAACCCGTTTCTGTCGTCAAAAAGGCGTATCAGAAACTGTGC
TACATTCATCATATCATGGGAAAGGTGCCAGACGCTTGCACTGCCTGCGATCTGGTCA
ATGTGGATTTGGATGACTGCATCTTTGAACAATAAATGATTTAAATCAGGTATGGCTG
CCGATGGTTATCTTCCAGattggctcgaggacactctctctgaaggaataagacagtggtggaagctcaaacctggcccacc
accaccaaagcccgcagagcggcataaggacgacagcaggggtcttgtgcttcctgggtacaagtacctcggacccttcaacggactcga
caagggagagccggtcaacgaggcagacgccgcggccctcgagcacgacaaagcctacgaccggcagctcgacagcggagacaacc
cgtacctcaagtacaaccacgccgacgcggagtttcaggagcgccttaaagaagatacgtcttttgggggcaacctcggacgagcagtcttc
caggcgaaaaagagggttcttgaacctctgggcctggttgaggaacctgttaagacggctccgggaaaaaagaggccggtagagcactctc
ctgtggagccagactcctcctcgggaaccggaaaggcgggccagcagcctgcaagaaaaagattgaattttggtcagactggagacgcag
actcagtacctgacccccagcctctcggacagccaccagcagccccctctggtctgggaactaatacgatggctacaggcagtggcgcacc
aatggcagacaataacgagggcgccgacggagtgggtaattcctcgggaaattggcattgcgattccacatggatgggcgacagagtcatc
accaccagcacccgaacctgggccctgcccacctacaacaaccacctctacaaacaaatttccagccaatcaggagcctcgaacgacaatc
actactttggctacagcaccccttgggggtattttgacttcaacagattccactgccacttttcaccacgtgactggcaaagactcatcaacaaca
actggggattccgacccaagagactcaacttcaagctctttaacattcaagtcaaagaggtcacgcagaatgacggtacgacgacgattgcc
aataaccttaccagcacggttcaggtgtttactgactcggagtaccagctcccgtacgtcctcggctcggcgcatcaaggatgcctcccgccg
ttcccagcagacgtcttcatggtgccacagtatggatacctcaccctgaacaacgggagtcaggcagtaggacgctcttcattttactgcctgg
agtactttccttctcagatgctgcgtaccggaaacaactttaccttcagctacacttttgaggacgttcctttccacagcagctacgctcacagcc
agagtctggaccgtctcatgaatcctctcatcgaccagtacctgtattacttgagcagaacaaacactccaagtggaaccaccacgcagtcaa
ggcttcagttttctcaggccggagcgagtgacattcgggaccagtctaggaactggcttcctggaccctgttaccgccagcagcgagtatcaa
agacatctgcggataacaacaacagtgaatactcgtggactggagctaccaagtaccacctcaatggcagagactctctggtgaatccgggc
ccggccatggcaagccacaaggacgatgaagaaaagttttttcctcagagcggggttctcatctttgggaagcaaggctcagagaaaacaaa
tgtggacattgaaaaggtcatgattacagacgaagaggaaatcaggacaaccaatcccgtggctacggagcagtatggttctgtatctaccaa
cctccagagaggcaacagacaagcagctaccgcagatgtcaacacacaaggcgttcttccaggcatggtctggcaggacagagatgtgta
ccttcaggggcccatctgggcaaagattccacacacggacggacattttcacccctctcccctcatgggggattcggacttaaacaccctcct
ccacagattctcatcaagaacaccccggtacctgcgaatccttcgaccaccttcagtgcggcaaagtttgcttccttcatcacacagtactccac
gggacaggtcagcgtggagatcgagtgggagctgcagaaggaaaacagcaaacgctggaatcccgaaattcagtacacttccaactacaa
caagtctgttaatgtggactttactgtggacactaatggcgtgtattcagagcctcgccccattggcaccagatacctgactcgtaatctgtaatt
gcttgttaatcaataaaccgtttaattcgtttcagttgaactttggtctctgcgtatttctttcttatctagtttccatggctacgtagataagtagcatgg
cgggttaatcattaactacaTAAGGGCGAATTCTGCAGATATCCATCACACTGGCGGCCGCTCGA
GCATGCATCTAGAGGGCCCAATTCGCCCTATAGTGAGTCGTATTACAATTCACTGGCC
GTCGTTTTACAACGTCGTGACTGGGAAAACCCTGGCGTTACCCAACTTAATCGCCTTG
CAGCACATCCCCCTTTCGCCAGCTGGCGTAATAGCGAAGAGGCCCGCACCGATCGCC
CTTCCCAACAGTTGCGCAGCCTATACGTACGGCAGTTTAAGGTTTACACCTATAAAAG
AGAGAGCCGTTATCGTCTGTTTGTGGATGTACAGAGTGATATTATTGACACGCCGGG
GCGACGGATGGTGATCCCCCTGGCCAGTGCACGTCTGCTGTCAGATAAAGTCTCCCGT
GAACTTTACCCGGTGGTGCATATCGGGGATGAAAGCTGGCGCATGATGACCACCGAT
ATGGCCAGTGTGCCGGTCTCCGTTATCGGGGAAGAAGTGGCTGATCTCAGCCACCGC
GAAAATGACATCAAAAACGCCATTAACCTGATGTTCTGGGGAATATAAATGTCAGGC
ATGAGATTATCAAAAAGGATCTTCACCTAGATCCTTTTCACGTAGAAAGCCAGTCCGC
AGAAACGGTGCTGACCCCGGATGAATGTCAGCTACTGGGCTATCTGGACAAGGGAAA
ACGCAAGCGCAAAGAGAAAGCAGGTAGCTTGCAGTGGGCTTACATGGCGATAGCTA
GACTGGGCGGTTTTATGGACAGCAAGCGAACCGGAATTGCCAGCTGGGGCGCCCTCT
GGTAAGGTTGGGAAGCCCTGCAAAGTAAACTGGATGGCTTTCTCGCCGCCAAGGATC
TGATGGCGCAGGGGATCAAGCTCTGATCAAGAGACAGGATGAGGATCGTTTCGCATG
ATTGAACAAGATGGATTGCACGCAGGTTCTCCGGCCGCTTGGGTGGAGAGGCTATTC
GGCTATGACTGGGCACAACAGACAATCGGCTGCTCTGATGCCGCCGTGTTCCGGCTG
TCAGCGCAGGGGCGCCCGGTTCTTTTTGTCAAGACCGACCTGTCCGGTGCCCTGAATG
AACTGCAAGACGAGGCAGCGCGGCTATCGTGGCTGGCCACGACGGGCGTTCCTTGCG
CAGCTGTGCTCGACGTTGTCACTGAAGCGGGAAGGGACTGGCTGCTATTGGGCGAAG
TGCCGGGGCAGGATCTCCTGTCATCTCACCTTGCTCCTGCCGAGAAAGTATCCATCAT
GGCTGATGCAATGCGGCGGCTGCATACGCTTGATCCGGCTACCTGCCCATTCGACCAC
CAAGCGAAACATCGCATCGAGCGAGCACGTACTCGGATGGAAGCCGGTCTTGTCGAT
CAGGATGATCTGGACGAAGAGCATCAGGGGCTCGCGCCAGCCGAACTGTTCGCCAGG
CTCAAGGCGAGCATGCCCGACGGCGAGGATCTCGTCGTGACCCATGGCGATGCCTGC
TTGCCGAATATCATGGTGGAAAATGGCCGCTTTTCTGGATTCATCGACTGTGGCCGGC
TGGGTGTGGCGGACCGCTATCAGGACATAGCGTTGGCTACCCGTGATATTGCTGAAG
AGCTTGGCGGCGAATGGGCTGACCGCTTCCTCGTGCTTTACGGTATCGCCGCTCCCGA
TTCGCAGCGCATCGCCTTCTATCGCCTTCTTGACGAGTTCTTCTGAATTATTAACGCTT
ACAATTTCCTGATGCGGTATTTTCTCCTTACGCATCTGTGCGGTATTTCACACCGCATA
CAGGTGGCACTTTTCGGGGAAATGTGCGCGGAACCCCTATTTGTTTATTTTTCTAAAT
ACATTCAAATATGTATCCGCTCATGAGACAATAACCCTGATAAATGCTTCAATAATAG
CACGTGAGGAGGGCCACCATGGCCAAGTTGACCAGTGCCGTTCCGGTGCTCACCGCG
CGCGACGTCGCCGGAGCGGTCGAGTTCTGGACCGACCGGCTCGGGTTCTCCCGGGAC
TTCGTGGAGGACGACTTCGCCGGTGTGGTCCGGGACGACGTGACCCTGTTCATCAGC
GCGGTCCAGGACCAGGTGGTGCCGGACAACACCCTGGCCTGGGTGTGGGTGCGCGGC
CTGGACGAGCTGTACGCCGAGTGGTCGGAGGTCGTGTCCACGAACTTCCGGGACGCC
TCCGGGCCGGCCATGACCGAGATCGGCGAGCAGCCGTGGGGGCGGGAGTTCGCCCTG
CGCGACCCGGCCGGCAACTGCGTGCACTTCGTGGCCGAGGAGCAGGACTGACACGTG
CTAAAACTTCATTTTTAATTTAAAAGGATCTAGGTGAAGATCCTTTTTGATAATCTCA
TGACCAAAATCCCTTAACGTGAGTTTTCGTTCCACTGAGCGTCAGACCCCGTAGAAAA
GATCAAAGGATCTTCTTGAGATCCTTTTTTTCTGCGCGTAATCTGCTGCTTGCAAACA
AAAAAACCACCGCTACCAGCGGTGGTTTGTTTGCCGGATCAAGAGCTACCAACTCTTT
TTCCGAAGGTAACTGGCTTCAGCAGAGCGCAGATACCAAATACTGTCCTTCTAGTGTA
GCCGTAGTTAGGCCACCACTTCAAGAACTCTGTAGCACCGCCTACATACCTCGCTCTG
CTAATCCTGTTACCAGTGGCTGCTGCCAGTGGCGATAAGTCGTGTCTTACCGGGTTGG
ACTCAAGACGATAGTTACCGGATAAGGCGCAGCGGTCGGGCTGAACGGGGGGTTCGT
GCACACAGCCCAGCTTGGAGCGAACGACCTACACCGAACTGAGATACCTACAGCGTG
AGCTATGAGAAAGCGCCACGCTTCCCGAAGGGAGAAAGGCGGACAGGTATCCGGTA
AGCGGCAGGGTCGGAACAGGAGAGCGCACGAGGGAGCTTCCAGGGGGAAACGCCTG
GTATCTTTATAGTCCTGTCGGGTTTCGCCACCTCTGACTTGAGCGTCGATTTTTGTGAT
GCTCGTCAGGGGGGCGGAGCCTATGGAAAAACGCCAGCAACGCGGCCTTTTTACGGT
TCCTGGGCTTTTGCTGGCCTTTTGCTCACATGTTCTTTCCTGCGTTATCCCCTGATTCTG
TGGATAACCGTATTACCGCCTTTGAGTGAGCTGATACCGCTCGCCGCAGCCGAACGA
CCGAGCGCAGCGAGTCAGTGAGCGAGGAAGCGGAAGAGCGCCCAATACGCAAACCG
CCTCTCCCCGCGCGTTGGCCGATTCATTAATGCAGCTGGCACGACAGGTTTCCCGACT
GGAAAGCGGGCAGTGAGCGCAACGCAATTAATGTGAGTTAGCTCACTCATTAGGCAC
CCCAGGCTTTACACTTTATGCTTCCGGCTCGTATGTTGTGTGGAATTGTGAGCGGATA
ACAATTTCACACAGGAAACAGCTATGACCATGATTACGCCAAGCTATTTAGGTGACA
CTATAGAATACTCAAGCTATGCATCAAGCTTGGTACCGAGCTCGGATCCACTAGTAA
CGGCCGCCAGTGTGCTGGAATTCGCCCT
SEQ ID NO: 7 (Rep/Cap construct for AAV2)
GGCCTCCACGGCCACTAGTAACGGCCGCCAGTGTGCTGGAATTCGCCCTG
GAGGGGTGGAGTCGTGACGTGAATTACGTCATAGGGTTAGGGAGGTCCTGTATTAGA
GGTCACGTGAGTGTTTTGCGACATTTTGCGACACCATGTGGTCACGCTGGGTATTTAA
GCCCGAGTGAGCACGCAGGGTCTCCATTTTGAAGCGGGAGGTTTGAACGCGCAGCCG
CCATGCCGGGGTTTTACGAGATTGTGATTAAGGTCCCCAGCGACCTTGACGAGCATCT
GCCCGGCATTTCTGACAGCTTTGTGAACTGGGTGGCCGAGAAGGAATGGGAGTTGCC
GCCAGATTCTGACATGGATCTGAATCTGATTGAGCAGGCACCCCTGACCGTGGCCGA
GAAGCTGCAGCGCGACTTTCTGACGGAATGGCGCCGTGTGAGTAAGGCCCCGGAGGC
CCTTTTCTTTGTGCAATTTGAGAAGGGAGAGAGCTACTTCCACATGCACGTGCTCGTG
GAAACCACCGGGGTGAAATCCATGGTTTTGGGACGTTTCCTGAGTCAGATTCGCGAA
AAACTGATTCAGAGAATTTACCGCGGGATCGAGCCGACTTTGCCAAACTGGTTCGCG
GTCACAAAGACCAGAAATGGCGCCGGAGGCGGGAACAAGGTGGTGGATGAGTGCTA
CATCCCCAATTACTTGCTCCCCAAAACCCAGCCTGAGCTCCAGTGGGCGTGGACTAAT
ATGGAACAGTATTTAAGCGCCTGTTTGAATCTCACGGAGCGTAAACGGTTGGTGGCG
CAGCATCTGACGCACGTGTCGCAGACGCAGGAGCAGAACAAAGAGAATCAGAATCC
CAATTCTGATGCGCCGGTGATCAGATCAAAAACTTCAGCCAGGTACATGGAGCTGGT
CGGGTGGCTCGTGGACAAGGTGAGTTTGGGGACCCTTGATTGTTCTTTCTTTTTCGCT
ATTGTAAAATTCATGTTATATGGAGGGGGCAAAGTTTTCAGGGTGTTGTTTAGAATGG
GAAGATGTCCCTTGTATCACCATGGACCCTCATGATAATTTTGTTTCTTTCACTTTCTA
CTCTGTTGACAACCATTGTCTCCTCTTATTTTCTTTTCATTTTCTGTAACTTTTTCGTTA
AACTTTAGCTTGCATTTGTAACGAATTTTTAAATTCACTTTTGTTTATTTGTCAGATTG
TAAGTACTTTCTCTAATCACTTTTTTTTCAAGGCAATCAGGGTATATTATATTGTACTT
CAGCACAGTTTTAGAGAACATAACTTCGTATAATGTATGCTATACGAAGTTATCGGGC
CCCTCTGCTAACCATGTTCATGCCTTCTTCTTTTTCCTACAGATGGTGAGCAAGGGCG
AGGAGCTGTTCACCGGGGTGGTGCCCATCCTGGTCGAGCTGGACGGCGACGTAAACG
GCCACAAGTTCAGCGTGTCCGGCGAGGGCGAGGGCGATGCCACCTACGGCAAGCTGA
CCCTGAAGTTCATCTGCACCACCGGCAAGCTGCCCGTGCCCTGGCCCACCCTCGTGAC
CACCCTGACCTACGGCGTGCAGTGCTTCAGCCGCTACCCCGACCACATGAAGCAGCA
CGACTTCTTCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGACACCCT
GGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGC
AGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTG
CTGCCCGACAACCACTACCTGAGCACCCAGTCCGCCCTGAGCAAAGACCCCAACGAG
AAGCGCGATCACATGGTCCTGCTGGAGTTCGTGACCGCCGCCGGGATCACTCTCGGC
ATGGACGAGCTGTACAAGTAACCTCAGGTGCAGGCTGCCTATCAGAAGGTGGTGGCT
GGTGTGGCCAATGCCCTGGCTCACAAATACCACTGAGATCTTTTTCCCTCTGCCAAAA
ATTATGGGGACATCATGAAGCCCCTTGAGCATCTGACTTCTGGCTAATAAAGGAAAT
TTATTTTCATTGCAATAGTGTGTTGGAATTTTTTGTGTCTCTCACTCGGAAGGACATAT
GGGAGGGCAAATCATTTAAAACATCAGAATGAGTATTTGGTTTAGAGTTTGGCAACA
TATGCCCATATGCTGGCTGCCATGAACAAAGGTTGGCTATAAAGAGGTCATCAGTAT
ATGAAACAGCCCCCTGCTGTCCATTCCTTATTCCATAGAAAAGCCTTGACTTGAGGTT
AGATTTTTTTTATATTTTGTTTTGTGTTATTTTTTTCTTTAACATCCCTAAAATTTTCCTT
ACATGTTTTACTAGCCAGATTTTTCCTCCTCTCCTGACTACTCCCAGTCATAGCTGTCC
CTCTTCTCTTATGGAGATCATAACTTCGTATAATGTATGCTATACGAAGTTATAATTGT
TATAATTAAATGATAAGGTAGAATATTTCTGCATATAAATTCTGGCTGGCGTGGAAAT
ATTCTTATTGGTAGAAACAACTACACCCTGGTCATCATCCTGCCTTTCTCTTTATGGTT
ACAATGATATACACTGTTTGAGATGAGGATAAAATACTCTGAGTCCAAACCGGGCCC
CTCTGCTAACCATGTTCATGCCTTCTTCTTTTTCCTACAGGGGATTACCTCGGAGAAGC
AGTGGATCCAGGAGGACCAGGCCTCATACATCTCCTTCAATGCGGCCTCCAACTCGC
GGTCCCAAATCAAGGCTGCCTTGGACAATGCGGGAAAGATTATGAGCCTGACTAAAA
CCGCCCCCGACTACCTGGTGGGCCAGCAGCCCGTGGAGGACATTTCCAGCAATCGGA
TTTATAAAATTTTGGAACTAAACGGGTACGATCCCCAATATGCGGCTTCCGTCTTTCT
GGGATGGGCCACGAAAAAGTTCGGCAAGAGGAACACCATCTGGCTGTTTGGGCCTGC
AACTACCGGGAAGACCAACATCGCGGAGGCCATAGCCCACACTGTGCCCTTCTACGG
GTGCGTAAACTGGACCAATGAGAACTTTCCCTTCAACGACTGTGTCGACAAGATGGT
GATCTGGTGGGAGGAGGGGAAGATGACCGCCAAGGTCGTGGAGTCGGCCAAAGCCA
TTCTCGGAGGAAGCAAGGTGCGCGTGGACCAGAAATGCAAGTCCTCGGCCCAGATAG
ACCCGACTCCCGTGATCGTCACCTCCAACACCAACATGTGCGCCGTGATTGACGGGA
ACTCAACGACCTTCGAACACCAGCAGCCGTTGCAAGACCGGATGTTCAAATTTGAAC
TCACCCGCCGTCTGGATCATGACTTTGGGAAGGTCACCAAGCAGGAAGTCAAAGACT
TTTTCCGGTGGGCAAAGGATCACGTGGTTGAGGTGGAGCATGAATTCTACGTCAAAA
AGGGTGGAGCCAAGAAAAGACCCGCCCCCAGTGACGCAGATATAAGTGAGCCCAAA
CGGGTGCGCGAGTCAGTTGCGCAGCCATCGACGTCAGACGCGGAAGCTTCGATCAAC
TACGCAGACAGGTACCAAAACAAATGTTCTCGTCACGTGGGCATGAATCTGATGCTG
TTTCCCTGCAGACAATGCGAGAGAATGAATCAGAATTCAAATATCTGCTTCACTCACG
GACAGAAAGACTGTTTAGAGTGCTTTCCCGTGTCAGAATCTCAACCCGTTTCTGTCGT
CAAAAAGGCGTATCAGAAACTGTGCTACATTCATCATATCATGGGAAAGGTGCCAGA
CGCTTGCACTGCCTGCGATCTGGTCAATGTGGATTTGGATGACTGCATCTTTGAACAA
TAAATGATTTAAATCAGGTATGGCTGCCGATGGTTATCTTCCAGATTGGCTCGAGGAC
ACTCTCTCTGAAGGAATAAGACAGTGGTGGAAGCTCAAACCTGGCCCACCACCACCA
AAGCCCGCAGAGCGGCATAAGGACGACAGCAGGGGTCTTGTGCTTCCTGGGTACAAG
TACCTCGGACCCTTCAACGGACTCGACAAGGGAGAGCCGGTCAACGAGGCAGACGCC
GCGGCCCTCGAGCACGACAAAGCCTACGACCGGCAGCTCGACAGCGGAGACAACCC
GTACCTCAAGTACAACCACGCCGACGCGGAGTTTCAGGAGCGCCTTAAAGAAGATAC
GTCTTTTGGGGGCAACCTCGGACGAGCAGTCTTCCAGGCGAAAAAGAGGGTTCTTGA
ACCTCTGGGCCTGGTTGAGGAACCTGTTAAGACGGCTCCGGGAAAAAAGAGGCCGGT
AGAGCACTCTCCTGTGGAGCCAGACTCCTCCTCGGGAACCGGAAAGGCGGGCCAGCA
GCCTGCAAGAAAAAGATTGAATTTTGGTCAGACTGGAGACGCAGACTCAGTACCTGA
CCCCCAGCCTCTCGGACAGCCACCAGCAGCCCCCTCTGGTCTGGGAACTAATACGAT
GGCTACAGGCAGTGGCGCACCAATGGCAGACAATAACGAGGGCGCCGACGGAGTGG
GTAATTCCTCGGGAAATTGGCATTGCGATTCCACATGGATGGGCGACAGAGTCATCA
CCACCAGCACCCGAACCTGGGCCCTGCCCACCTACAACAACCACCTCTACAAACAAA
TTTCCAGCCAATCAGGAGCCTCGAACGACAATCACTACTTTGGCTACAGCACCCCTTG
GGGGTATTTTGACTTCAACAGATTCCACTGCCACTTTTCACCACGTGACTGGCAAAGA
CTCATCAACAACAACTGGGGATTCCGACCCAAGAGACTCAACTTCAAGCTCTTTAAC
ATTCAAGTCAAAGAGGTCACGCAGAATGACGGTACGACGACGATTGCCAATAACCTT
ACCAGCACGGTTCAGGTGTTTACTGACTCGGAGTACCAGCTCCCGTACGTCCTCGGCT
CGGCGCATCAAGGATGCCTCCCGCCGTTCCCAGCAGACGTCTTCATGGTGCCACAGT
ATGGATACCTCACCCTGAACAACGGGAGTCAGGCAGTAGGACGCTCTTCATTTTACT
GCCTGGAGTACTTTCCTTCTCAGATGCTGCGTACCGGAAACAACTTTACCTTCAGCTA
CACTTTTGAGGACGTTCCTTTCCACAGCAGCTACGCTCACAGCCAGAGTCTGGACCGT
CTCATGAATCCTCTCATCGACCAGTACCTGTATTACTTGAGCAGAACAAACACTCCAA
GTGGAACCACCACGCAGTCAAGGCTTCAGTTTTCTCAGGCCGGAGCGAGTGACATTC
GGGACCAGTCTAGGAACTGGCTTCCTGGACCCTGTTACCGCCAGCAGCGAGTATCAA
AGACATCTGCGGATAACAACAACAGTGAATACTCGTGGACTGGAGCTACCAAGTACC
ACCTCAATGGCAGAGACTCTCTGGTGAATCCGGGCCCGGCCATGGCAAGCCACAAGG
ACGATGAAGAAAAGTTTTTTCCTCAGAGCGGGGTTCTCATCTTTGGGAAGCAAGGCT
CAGAGAAAACAAATGTGGACATTGAAAAGGTCATGATTACAGACGAAGAGGAAATC
AGGACAACCAATCCCGTGGCTACGGAGCAGTATGGTTCTGTATCTACCAACCTCCAG
AGAGGCAACAGACAAGCAGCTACCGCAGATGTCAACACACAAGGCGTTCTTCCAGGC
ATGGTCTGGCAGGACAGAGATGTGTACCTTCAGGGGCCCATCTGGGCAAAGATTCCA
CACACGGACGGACATTTTCACCCCTCTCCCCTCATGGGTGGATTCGGACTTAAACACC
CTCCTCCACAGATTCTCATCAAGAACACCCCGGTACCTGCGAATCCTTCGACCACCTT
CAGTGCGGCAAAGTTTGCTTCCTTCATCACACAGTACTCCACGGGACAGGTCAGCGT
GGAGATCGAGTGGGAGCTGCAGAAGGAAAACAGCAAACGCTGGAATCCCGAAATTC
AGTACACTTCCAACTACAACAAGTCTGTTAATGTGGACTTTACTGTGGACACTAATGG
CGTGTATTCAGAGCCTCGCCCCATTGGCACCAGATACCTGACTCGTAATCTGTAATTG
CTTGTTAATCAATAAACCGTTTAATTCGTTTCAGTTGAACTTTGGTCTCTGCGTATTTC
TTTCTTATCTAGTTTCCATGGCTACGTAGATAAGTAGCATGGCGGGTTAATCATTAAC
TACA
SEQ ID NO: 8 (STXC0068)
ACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCAC
ATTTCCCCGAAAAGTGCCACCTAAATTGTAAGCGTTAATATTTTGTTAAAATTCGCGT
TAAATTTTTGTTAAATCAGCTCATTTTTTAACCAATAGGCCGAAATCGGCAAAATCCC
TTATAAATCAAAAGAATAGACCGAGATAGGGTTGAGTGTTGTTCCAGTTTGGAACAA
GAGTCCACTATTAAAGAACGTGGACTCCAACGTCAAAGGGCGAAAAACCGTCTATCA
GGGCGATGGCCCACTACGTGAACCATCACCCTAATCAAGTTTTTTGGGGTCGAGGTG
CCGTAAAGCACTAAATCGGAACCCTAAAGGGAGCCCCCGATTTAGAGCTTGACGGGG
AAAGCCGGCGAACGTGGCGAGAAAGGAAGGGAAGAAAGCGAAAGGAGCGGGCGCT
AGGGCGCTGGCAAGTGTAGCGGTCACGCTGCGCGTAACCACCACACCCGCCGCGCTT
AATGCGCCGCTACAGGGCGCGTCCCATTCGCCATTCAGGCTGCGCAACTGTTGGGAA
GGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACG
ACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACG
CTGCAGAACACGCAGCTAGATTAACCCTAGAAAGATAATCATATTGTGACGTACGTT
AAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATA
AATTCAACAAACAATTTATTTATGTTTATTTATTTATTAAAAAAAACAAAAACTCAAA
ATTTCTTCTATAAAGTAACAAAACTTTTATGAGGGACAGCCCCCCCCCAAAGCCCCCA
GGGATGTAATTACGTCCCTCCCCCGCTAGGGGGCAGCAGCGAGCCGCCCGGGGCTCC
GCTCCGGTCCGGCGCTCCCCCCGCATCCCCGAGCCGGCAGCGTGCGGGGACAGCCCG
GGCACGGGGAAGGTGGCACGGGATCGCTTTCCTCTGAACGCTTCTCGCTGCTCTTTGA
GCCTGCAGACACCTGGGGGGATACGGGGAAAAGGCCTCCACGGCCACTAGTAACGG
CCGCCAGTGTGCTGGAATTCGCCCTGGAGGGGTGGAGTCGTGACGTGAATTACGTCA
TAGGGTTAGGGAGGTCCTGTATTAGAGGTCACGTGAGTGTTTTGCGACATTTTGCGAC
ACCATGTGGTCACGCTGGGTATTTAAGCCCGAGTGAGCACGCAGGGTCTCCATTTTGA
AGCGGGAGGTTTGAACGCGCAGCCGCCATGCCGGGGTTTTACGAGATTGTGATTAAG
GTCCCCAGCGACCTTGACGAGCATCTGCCCGGCATTTCTGACAGCTTTGTGAACTGGG
TGGCCGAGAAGGAATGGGAGTTGCCGCCAGATTCTGACATGGATCTGAATCTGATTG
AGCAGGCACCCCTGACCGTGGCCGAGAAGCTGCAGCGCGACTTTCTGACGGAATGGC
GCCGTGTGAGTAAGGCCCCGGAGGCCCTTTTCTTTGTGCAATTTGAGAAGGGAGAGA
GCTACTTCCACATGCACGTGCTCGTGGAAACCACCGGGGTGAAATCCATGGTTTTGG
GACGTTTCCTGAGTCAGATTCGCGAAAAACTGATTCAGAGAATTTACCGCGGGATCG
AGCCGACTTTGCCAAACTGGTTCGCGGTCACAAAGACCAGAAATGGCGCCGGAGGCG
GGAACAAGGTGGTGGATGAGTGCTACATCCCCAATTACTTGCTCCCCAAAACCCAGC
CTGAGCTCCAGTGGGCGTGGACTAATATGGAACAGTATTTAAGCGCCTGTTTGAATCT
CACGGAGCGTAAACGGTTGGTGGCGCAGCATCTGACGCACGTGTCGCAGACGCAGGA
GCAGAACAAAGAGAATCAGAATCCCAATTCTGATGCGCCGGTGATCAGATCAAAAAC
TTCAGCCAGGTACATGGAGCTGGTCGGGTGGCTCGTGGACAAGGTGAGTTTGGGGAC
CCTTGATTGTTCTTTCTTTTTCGCTATTGTAAAATTCATGTTATATGGAGGGGGCAAAG
TTTTCAGGGTGTTGTTTAGAATGGGAAGATGTCCCTTGTATCACCATGGACCCTCATG
ATAATTTTGTTTCTTTCACTTTCTACTCTGTTGACAACCATTGTCTCCTCTTATTTTCTT
TTCATTTTCTGTAACTTTTTCGTTAAACTTTAGCTTGCATTTGTAACGAATTTTTAAATT
CACTTTTGTTTATTTGTCAGATTGTAAGTACTTTCTCTAATCACTTTTTTTTCAAGGCA
ATCAGGGTATATTATATTGTACTTCAGCACAGTTTTAGAGAACATAACTTCGTATAAT
GTATGCTATACGAAGTTATCGGGCCCCTCTGCTAACCATGTTCATGCCTTCTTCTTTTT
CCTACAGATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGGGCGAGG
GCGATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAGCCG
CTACCCCGACCACATGAAGCAGCACGACTTCTTCAAGTCCGCCATGCCCGAAGGCTA
CGTCCAGGAGCGCACCATCTTCTTCAAGGACGACGGCAACTACAAGACCCGCGCCGA
GGTGAAGTTCGAGGGCGACACCCTGGTGAACCGCATCGAGCTGAAGGGCATCGACTT
CAAGGAGGACGGCAACATCCTGGGGCACAAGCTGGAGTACAACTACAACAGCCACA
ACGTCTATATCATGGCCGACAAGCAGAAGAACGGCATCAAGGTGAACTTCAAGATCC
GCCACAACATCGAGGACGGCAGCGTGCAGCTCGCCGACCACTACCAGCAGAACACCC
CCATCGGCGACGGCCCCGTGCTGCTGCCCGACAACCACTACCTGAGCACCCAGTCCG
CCCTGAGCAAAGACCCCAACGAGAAGCGCGATCACATGGTCCTGCTGGAGTTCGTGA
CCGCCGCCGGGATCACTCTCGGCATGGACGAGCTGTACAAGTAACCTCAGGTGCAGG
CTGCCTATCAGAAGGTGGTGGCTGGTGTGGCCAATGCCCTGGCTCACAAATACCACT
GAGATCTTTTTCCCTCTGCCAAAAATTATGGGGACATCATGAAGCCCCTTGAGCATCT
GACTTCTGGCTAATAAAGGAAATTTATTTTCATTGCAATAGTGTGTTGGAATTTTTTGT
GTCTCTCACTCGGAAGGACATATGGGAGGGCAAATCATTTAAAACATCAGAATGAGT
ATTTGGTTTAGAGTTTGGCAACATATGCCCATATGCTGGCTGCCATGAACAAAGGTTG
GCTATAAAGAGGTCATCAGTATATGAAACAGCCCCCTGCTGTCCATTCCTTATTCCAT
AGAAAAGCCTTGACTTGAGGTTAGATTTTTTTTATATTTTGTTTTGTGTTATTTTTTTCT
TTAACATCCCTAAAATTTTCCTTACATGTTTTACTAGCCAGATTTTTCCTCCTCTCCTG
ACTACTCCCAGTCATAGCTGTCCCTCTTCTCTTATGGAGATCATAACTTCGTATAATGT
ATGCTATACGAAGTTATAATTGTTATAATTAAATGATAAGGTAGAATATTTCTGCATA
TAAATTCTGGCTGGCGTGGAAATATTCTTATTGGTAGAAACAACTACACCCTGGTCAT
CATCCTGCCTTTCTCTTTATGGTTACAATGATATACACTGTTTGAGATGAGGATAAAA
TACTCTGAGTCCAAACCGGGCCCCTCTGCTAACCATGTTCATGCCTTCTTCTTTTTCCT
ACAGGGGATTACCTCGGAGAAGCAGTGGATCCAGGAGGACCAGGCCTCATACATCTC
CTTCAATGCGGCCTCCAACTCGCGGTCCCAAATCAAGGCTGCCTTGGACAATGCGGG
AAAGATTATGAGCCTGACTAAAACCGCCCCCGACTACCTGGTGGGCCAGCAGCCCGT
GGAGGACATTTCCAGCAATCGGATTTATAAAATTTTGGAACTAAACGGGTACGATCC
CCAATATGCGGCTTCCGTCTTTCTGGGATGGGCCACGAAAAAGTTCGGCAAGAGGAA
CACCATCTGGCTGTTTGGGCCTGCAACTACCGGGAAGACCAACATCGCGGAGGCCAT
AGCCCACACTGTGCCCTTCTACGGGTGCGTAAACTGGACCAATGAGAACTTTCCCTTC
AACGACTGTGTCGACAAGATGGTGATCTGGTGGGAGGAGGGGAAGATGACCGCCAA
GGTCGTGGAGTCGGCCAAAGCCATTCTCGGAGGAAGCAAGGTGCGCGTGGACCAGA
AATGCAAGTCCTCGGCCCAGATAGACCCGACTCCCGTGATCGTCACCTCCAACACCA
ACATGTGCGCCGTGATTGACGGGAACTCAACGACCTTCGAACACCAGCAGCCGTTGC
AAGACCGGATGTTCAAATTTGAACTCACCCGCCGTCTGGATCATGACTTTGGGAAGG
TCACCAAGCAGGAAGTCAAAGACTTTTTCCGGTGGGCAAAGGATCACGTGGTTGAGG
TGGAGCATGAATTCTACGTCAAAAAGGGTGGAGCCAAGAAAAGACCCGCCCCCAGT
GACGCAGATATAAGTGAGCCCAAACGGGTGCGCGAGTCAGTTGCGCAGCCATCGACG
TCAGACGCGGAAGCTTCGATCAACTACGCAGACAGGTACCAAAACAAATGTTCTCGT
CACGTGGGCATGAATCTGATGCTGTTTCCCTGCAGACAATGCGAGAGAATGAATCAG
AATTCAAATATCTGCTTCACTCACGGACAGAAAGACTGTTTAGAGTGCTTTCCCGTGT
CAGAATCTCAACCCGTTTCTGTCGTCAAAAAGGCGTATCAGAAACTGTGCTACATTCA
TCATATCATGGGAAAGGTGCCAGACGCTTGCACTGCCTGCGATCTGGTCAATGTGGA
TTTGGATGACTGCATCTTTGAACAATAAATGATTTAAATCAGGTATGGCTGCCGATGG
TTATCTTCCAGATTGGCTCGAGGACACTCTCTCTGAAGGAATAAGACAGTGGTGGAA
GCTCAAACCTGGCCCACCACCACCAAAGCCCGCAGAGCGGCATAAGGACGACAGCA
GGGGTCTTGTGCTTCCTGGGTACAAGTACCTCGGACCCTTCAACGGACTCGACAAGG
GAGAGCCGGTCAACGAGGCAGACGCCGCGGCCCTCGAGCACGACAAAGCCTACGAC
CGGCAGCTCGACAGCGGAGACAACCCGTACCTCAAGTACAACCACGCCGACGCGGA
GTTTCAGGAGCGCCTTAAAGAAGATACGTCTTTTGGGGGCAACCTCGGACGAGCAGT
CTTCCAGGCGAAAAAGAGGGTTCTTGAACCTCTGGGCCTGGTTGAGGAACCTGTTAA
GACGGCTCCGGGAAAAAAGAGGCCGGTAGAGCACTCTCCTGTGGAGCCAGACTCCTC
CTCGGGAACCGGAAAGGCGGGCCAGCAGCCTGCAAGAAAAAGATTGAATTTTGGTC
AGACTGGAGACGCAGACTCAGTACCTGACCCCCAGCCTCTCGGACAGCCACCAGCAG
CCCCCTCTGGTCTGGGAACTAATACGATGGCTACAGGCAGTGGCGCACCAATGGCAG
ACAATAACGAGGGCGCCGACGGAGTGGGTAATTCCTCGGGAAATTGGCATTGCGATT
CCACATGGATGGGCGACAGAGTCATCACCACCAGCACCCGAACCTGGGCCCTGCCCA
CCTACAACAACCACCTCTACAAACAAATTTCCAGCCAATCAGGAGCCTCGAACGACA
ATCACTACTTTGGCTACAGCACCCCTTGGGGGTATTTTGACTTCAACAGATTCCACTG
CCACTTTTCACCACGTGACTGGCAAAGACTCATCAACAACAACTGGGGATTCCGACC
CAAGAGACTCAACTTCAAGCTCTTTAACATTCAAGTCAAAGAGGTCACGCAGAATGA
CGGTACGACGACGATTGCCAATAACCTTACCAGCACGGTTCAGGTGTTTACTGACTCG
GAGTACCAGCTCCCGTACGTCCTCGGCTCGGCGCATCAAGGATGCCTCCCGCCGTTCC
CAGCAGACGTCTTCATGGTGCCACAGTATGGATACCTCACCCTGAACAACGGGAGTC
AGGCAGTAGGACGCTCTTCATTTTACTGCCTGGAGTACTTTCCTTCTCAGATGCTGCG
TACCGGAAACAACTTTACCTTCAGCTACACTTTTGAGGACGTTCCTTTCCACAGCAGC
TACGCTCACAGCCAGAGTCTGGACCGTCTCATGAATCCTCTCATCGACCAGTACCTGT
ATTACTTGAGCAGAACAAACACTCCAAGTGGAACCACCACGCAGTCAAGGCTTCAGT
TTTCTCAGGCCGGAGCGAGTGACATTCGGGACCAGTCTAGGAACTGGCTTCCTGGAC
CCTGTTACCGCCAGCAGCGAGTATCAAAGACATCTGCGGATAACAACAACAGTGAAT
ACTCGTGGACTGGAGCTACCAAGTACCACCTCAATGGCAGAGACTCTCTGGTGAATC
CGGGCCCGGCCATGGCAAGCCACAAGGACGATGAAGAAAAGTTTTTTCCTCAGAGCG
GGGTTCTCATCTTTGGGAAGCAAGGCTCAGAGAAAACAAATGTGGACATTGAAAAGG
TCATGATTACAGACGAAGAGGAAATCAGGACAACCAATCCCGTGGCTACGGAGCAGT
ATGGTTCTGTATCTACCAACCTCCAGAGAGGCAACAGACAAGCAGCTACCGCAGATG
TCAACACACAAGGCGTTCTTCCAGGCATGGTCTGGCAGGACAGAGATGTGTACCTTC
AGGGGCCCATCTGGGCAAAGATTCCACACACGGACGGACATTTTCACCCCTCTCCCCT
CATGGGTGGATTCGGACTTAAACACCCTCCTCCACAGATTCTCATCAAGAACACCCCG
GTACCTGCGAATCCTTCGACCACCTTCAGTGCGGCAAAGTTTGCTTCCTTCATCACAC
AGTACTCCACGGGACAGGTCAGCGTGGAGATCGAGTGGGAGCTGCAGAAGGAAAAC
AGCAAACGCTGGAATCCCGAAATTCAGTACACTTCCAACTACAACAAGTCTGTTAAT
GTGGACTTTACTGTGGACACTAATGGCGTGTATTCAGAGCCTCGCCCCATTGGCACCA
GATACCTGACTCGTAATCTGTAATTGCTTGTTAATCAATAAACCGTTTAATTCGTTTCA
GTTGAACTTTGGTCTCTGCGTATTTCTTTCTTATCTAGTTTCCATGGCTACGTAGATAA
GTAGCATGGCGGGTTAATCATTAACTACATAAGGGCGAATTCTGCAGATATCCATCA
CACTGGCGGCCGCTCGAGCATGCATCTAGAGCTAGCGAATTCGAATTTAAATCGGAT
CCGCGGCCGCAAGGATCTGCGATCGCTCCGGTGCCCGTCAGTGGGCAGAGCGCACAT
CGCCCACAGTCCCCGAGAAGTTGGGGGGAGGGGTCGGCAATTGAACGGGTGCCTAG
AGAAGGTGGCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACGTTCTTTTT
CGCAACGGGTTTGCCGCCAGAACACAGCTGAAGCTTCGAGGGGCTCGCATCTCTCCT
TCACGCGCCCGCCGCCCTACCTGAGGCCGCCATCCACGCCGGTTGAGTCGCGTTCTGC
CGCCTCCCGCCTGTGGTGCCTCCTGAACTGCGTCCGCCGTCTAGGTAAGTTTAAAGCT
CAGGTCGAGACCGGGCCTTTGTCCGGCGCTCCCTTGGAGCCTACCTAGACTCAGCCG
GCTCTCCACGCTTTGCCTGACCCTGCTTGCTCAACTCTACGTCTTTGTTTCGTTTTCTGT
TCTGCGCCGTTACAGATCCAAGCTGTGACCGGCGCCTACGATATCGCCACCATGAAA
ACATTTAACATTTCTCAACAGGATCTAGAATTAGTAGAAGTAGCGACAGAGAAGATT
ACAATGCTTTATGAGGATAATAAACATCATGTGGGAGCGGCAATTCGTACGAAAACA
GGAGAAATCATTTCGGCAGTACATATTGAAGCGTATATAGGACGAGTAACTGTTTGT
GCAGAAGCCATTGCGATTGGTAGTGCAGTTTCGAATGGACAAAAGGATTTTGACACG
ATTGTAGCTGTTAGACACCCTTATTCTGACGAAGTAGATAGAAGTATTCGAGTGGTAA
GTCCTTGTGGTATGTGTAGGGAGTTGATTTCAGACTATGCACCAGATTGTTTTGTGTT
AATAGAAATGAATGGCAAGTTAGTCAAAACTACGATTGAAGAACTCATTCCACTCAA
ATATACCCGAAATTAAGGTACCTCGACAACCTTCCAAACTGAGTGCATGACCCGCAA
GCCCGGTGCCTGAAATCAACCTCTGGATTACAAAATTTGTGAAAGATTGACTGGTATT
CTTAACTATGTTGCTCCTTTTACGCTATGTGGATACGCTGCTTTAATGCCTTTGTATCA
TGCGTTAACTAAACTTGTTTATTGCAGCTTATAATGGTTACAAATAAAGCAATAGCAT
CACAAATTTCACAAATAAAGCATTTTTTTCACTGCATTCTAGTTGTGGTTTGTCCAAA
CTCATCAATGTATCTTATCATGTCTGGAATTGACTCAAATGATGTCAATTAGTCTATC
AGAAGCTATCTGGTCTCCCTTCCGGGGGACAAGACATCCCTGTTTAATATTTAAACAG
CAGTGTTCCCAAACTGGGTTCTTATATCCCTTGCTCTGGTCAACCAGGTTGCAGGGTT
TCCTGTCCTCACAGGAACGAAGTCCCTAAAGAAACAGTGGCAGCCAGGTTTAGCCCC
GGAATTGACTGGATTCCTTTTTTAGGGCCCATTGGTATGGCTTTTTCCCCGTATCCCCC
CAGGTGTCTGCAGGCTCAAAGAGCAGCGAGAAGCGTTCAGAGGAAAGCGATCCCGT
GCCACCTTCCCCGTGCCCGGGCTGTCCCCGCACGCTGCCGGCTCGGGGATGCGGGGG
GAGCGCCGGACCGGAGCGGAGCCCCGGGCGGCTCGCTGCTGCCCCCTAGCGGGGGA
GGGACGTAATTACATCCCTGGGGGCTTTGGGGGGGGGCTGTCCCTGATATCTATAAC
AAGAAAATATATATATAATAAGTTATCACGTAAGTAGAACATGAAATAACAATATAA
TTATCGTATGAGTTAAATCTTAAAAGTCACGTAAAAGATAATCATGCGTCATTTTGAC
TCACGCGGTCGTTATAGTTCAAAATCAGTGACACTTACCGCATTGACAAGCACGCCTC
ACGGGAGCTCCAAGCGGCGACTGAGATGTCCTAAATGCACAGCGACGGATTCGCGCT
ATTTAGAAAGAGAGAGCAATATTTCAAGAATGCATGCGTCAATTTTACGCAGACTAT
CTTTCTAGGGTTAATCTAGCTGCATCAGGATCATATCGTCGGGTCTTTTTTCCGGCTCA
GTCATCGCCCAAGCTGGCGCTATCTGGGCATCGGGGAGGAAGAAGCCCGTGCCTTTT
CCCGCGAGGTTGAAGCGGCATGGAAAGAGTTTGCCGAGGATGACTGCTGCTGCATTG
ACGTTGAGCGAAAACGCACGTTTACCATGATGATTCGGGAAGGTGTGGCCATGCACG
CCTTTAACGGTGAACTGTTCGTTCAGGCCACCTGGGATACCAGTTCGTCGCGGCTTTT
CCGGACACAGTTCCGGATGGTCAGCCCGAAGCGCATCAGCAACCCGAACAATACCGG
CGACAGCCGGAACTGCCGTGCCGGTGTGCAGATTAATGACAGCGGTGCGGCGCTGGG
ATATTACGTCAGCGAGGACGGGTATCCTGGCTGGATGCCGCAGAAATGGACATGGAT
ACCCCGTGAGTTACCCGGCGGGCGCGCTTGGCGTAATCATGGTCATAGCTGTTTCCTG
TGTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCACT
GCCCGCTTTCCAGTCGGGAAACCTGTCGTGCCAGCTGCATTAATGAATCGGCCAACG
CGCGGGGAGAGGCGGTTTGCGTATTGGGCGCTCTTCCGCTTCCTCGCTCACTGACTCG
CTGCGCTCGGTCGTTCGGCTGCGGCGAGCGGTATCAGCTCACTCAAAGGCGGTAATA
CGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCG
CCCCCCTGACGAGCATCACAAAAATCGACGCTCAAGTCAGAGGTGGCGAAACCCGAC
AGGACTATAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGCGTGGCGC
TTTCTCATAGCTCACGCTGTAGGTATCTCAGTTCGGTGTAGGTCGTTCGCTCCAAGCT
GGGCTGTGTGCACGAACCCCCCGTTCAGCCCGACCGCTGCGCCTTATCCGGTAACTAT
CGTCTTGAGTCCAACCCGGTAAGACACGACTTATCGCCACTGGCAGCAGCCACTGGT
AACAGGATTAGCAGAGCGAGGTATGTAGGCGGTGCTACAGAGTTCTTGAAGTGGTGG
CCTAACTACGGCTACACTAGAAGGACAGTATTTGGTATCTGCGCTCTGCTGAAGCCA
GTTACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTCAA
GAAGATCCTTTGATCTTTTCTACGGGGTCTGACGCTCAGTGGAACGAAAACTCACGTT
AAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTAC
CAATGCTTAATCAGTGAGGCACCTATCTCAGCGATCTGTCTATTTCGTTCATCCATAG
TTGCCTGACTCCCCGTCGTGTAGATAACTACGATACGGGAGGGCTTACCATCTGGCCC
CAGTGCTGCAATGATACCGCGAGACCCACGCTCACCGGCTCCAGATTTATCAGCAAT
AAACCAGCCAGCCGGAAGGGCCGAGCGCAGAAGTGGTCCTGCAACTTTATCCGCCTC
CATCCAGTCTATTAATTGTTGCCGGGAAGCTAGAGTAAGTAGTTCGCCAGTTAATAGT
TTGCGCAACGTTGTTGCCATTGCTACAGGCATCGTGGTGTCACGCTCGTCGTTTGGTA
TGGCTTCATTCAGCTCCGGTTCCCAACGATCAAGGCGAGTTACATGATCCCCCATGTT
GTGCAAAAAAGCGGTTAGCTCCTTCGGTCCTCCGATCGTTGTCAGAAGTAAGTTGGCC
GCAGTGTTATCACTCATGGTTATGGCAGCACTGCATAATTCTCTTACTGTCATGCCAT
CCGTAAGATGCTTTTCTGTGACTGGTGAGTACTCAACCAAGTCATTCTGAGAATAGTG
TATGCGGCGACCGAGTTGCTCTTGCCCGGCGTCAATACGGGATAATACCGCGCCACA
TAGCAGAACTTTAAAAGTGCTCATCATTGGAAAACGTTCTTCGGGGCGAAAACTCTC
AAGGATCTTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGAAGGCAAA
ATGCCGCAAAAAAGGGAATAAGGGCGACACGGAAATGTTGAATACTCAT
SEQ ID NO: 9 (STXC0090)
ACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCAC
ATTTCCCCGAAAAGTGCCACCTAAATTGTAAGCGTTAATATTTTGTTAAAATTCGCGT
TAAATTTTTGTTAAATCAGCTCATTTTTTAACCAATAGGCCGAAATCGGCAAAATCCC
TTATAAATCAAAAGAATAGACCGAGATAGGGTTGAGTGTTGTTCCAGTTTGGAACAA
GAGTCCACTATTAAAGAACGTGGACTCCAACGTCAAAGGGCGAAAAACCGTCTATCA
GGGCGATGGCCCACTACGTGAACCATCACCCTAATCAAGTTTTTTGGGGTCGAGGTG
CCGTAAAGCACTAAATCGGAACCCTAAAGGGAGCCCCCGATTTAGAGCTTGACGGGG
AAAGCCGGCGAACGTGGCGAGAAAGGAAGGGAAGAAAGCGAAAGGAGCGGGCGCT
AGGGCGCTGGCAAGTGTAGCGGTCACGCTGCGCGTAACCACCACACCCGCCGCGCTT
AATGCGCCGCTACAGGGCGCGTCCCATTCGCCATTCAGGCTGCGCAACTGTTGGGAA
GGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACG
ACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACG
CTGCAGAACACGCAGCTAGATTAACCCTAGAAAGATAATCATATTGTGACGTACGTT
AAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATA
AATTCAACAAACAATTTATTTATGTTTATTTATTTATTAAAAAAAACAAAAACTCAAA
ATTTCTTCTATAAAGTAACAAAACTTTTATGAGGGACAGCCCCCCCCCAAAGCCCCCA
GGGATGTAATTACGTCCCTCCCCCGCTAGGGGGCAGCAGCGAGCCGCCCGGGGCTCC
GCTCCGGTCCGGCGCTCCCCCCGCATCCCCGAGCCGGCAGCGTGCGGGGACAGCCCG
GGCACGGGGAAGGTGGCACGGGATCGCTTTCCTCTGAACGCTTCTCGCTGCTCTTTGA
GCCTGCAGACACCTGGGGGGATACGGGGAAAAGGCCTCCACGGCCACTAGTCCATAG
AGCCCACCGCATCCCCAGCATGCCTGCTATTGTCTTCCCAATCCTCCCCCTTGCTGTCC
TGCCCCACCCCACCCCCTAGAATAGAATGACACCTACTCAGACAATGCGATGCAATT
TCCTCATTTTATTAGGAAAGGACAGTGGGAGTGGCACCTTCCAGGGTCAAGGAAGGC
ACGGGGGAGGGGCAAACAACAGATGGCTGGCAACTAGAAGGCACAGCTACATGGGG
GTAGAGTCATAATCGTGCATCAGGATAGGGCGGTGGTGCTGCAGCAGCGCGCGAATA
AACTGCTGCCGCCGCCGCTCCGTCCTGCAGGAATACAACATGGCAGTGGTCTCCTCA
GCGATGATTCGCACCGCCCGCAGCATGAGACGCCTTGTCCTCCGGGCACAGCAGCGC
ACCCTGATCTCACTTAAATCAGCACAGTAACTGCAGCACAGCACCACAATATTGTTCA
AAATCCCACAGTGCAAGGCGCTGTATCCAAAGCTCATGGCGGGGACCACAGAACCCA
CGTGGCCATCATACCACAAGCGCAGGTAGATTAAGTGGCGACCCCTCATAAACACGC
TGGACATAAACATTACCTCTTTTGGCATGTTGTAATTCACCACCTCCCGGTACCATAT
AAACCTCTGATTAAACATGGCGCCATCCACCACCATCCTAAACCAGCTGGCCAAAAC
CTGCCCGCCGGCTATGCACTGCAGGGAACCGGGACTGGAACAATGACAGTGGAGAG
CCCAGGACTCGTAACCATGGATCATCATGCTCGTCATGATATCAATGTTGGCACAACA
CAGGCACACGTGCATACACTTCCTCAGGATTACAAGCTCCTCCCGCGTCAGAACCAT
ATCCCAGGGAACAACCCATTCCTGAATCAGCGTAAATCCCACACTGCAGGGAAGACC
TCGCACGTAACTCACGTTGTGCATTGTCAAAGTGTTACATTCGGGCAGCAGCGGATG
ATCCTCCAGTATGGTAGCGCGGGTCTCTGTCTCAAAAGGAGGTAGGCGATCCCTACT
GTACGGAGTGCGCCGAGACAACCGAGATCGTGTTGGTCGTAGTGTCATGCCAAATGG
AACGCCGGACGTAGTCATGGTTGTGGCCATATTATCATCGTGTTTTTCAAAGGAAAAC
CACGTCCCCGTGGTTCGGGGGGCCTAGACGTTTTTTTAACCTCGACTAAACACATGTA
AAGCATGTGCACCGAGGCCCCAGATCAGATCCCATACAATGGGGTACCTTCTGGGCA
TCCTTCAGCCCCTTGTTGAATACGCTTGAGGAGAGCCATTTGACTCTTTCCACAACTA
TCCAACTCACAACGTGGCACTGGGGTTGTGCCGCCTTTGCAGGTGTATCTTATACACG
TGGCTTTTGGCCGCAGAGGCACCTGTCGCCAGGTGGGGGGTTCCGCTGCCTGCAAAG
GGTCGCTACAGACGTTGTTTGTCTTCAAGAAGCTTCCAGAGGAACTGCTTCCTTCACG
ACATTCAACAGACCTTGCATTCCTTTGGCGAGAGGGGAAAGACCCCTAGGAATGCTC
GTCAAGAAGACAGGGCCAGGTTTCCGGGCCCTCACATTGCCAAAAGACGGCAATATG
GTGGAAAATAACATATAGACAAACGCACACCGGCCTTATTCCAAGCGGCTTCGGCCA
GTAACGTTAGGGGGGGGGGAGGGAGAGGGGCTTAAAAATCAAAGGGGTTCTGCCGC
GCATCACTATGCGCCACTGGCAGGGACACGTTGCGATACTGGTGTTTAGTGCTCCACT
TAAACTCAGGCACAACCATCCGCGGCAGCTCGGTGAAGTTTTCACTCCACAGGCTGC
GCACCATCACCAACGCGTTTAGCAGGTCGGGCGCCGATATCTTGAAGTCGCAGTTGG
GGCCTCCGCCCTGCGCGCGCGAGTTGCGATACACAGGGTTGCAGCACTGGAACACTA
TCAGCGCCGGGTGGTGCACGCTGGCCAGCACGCTCTTGTCGGAGATCAGATCCGCGT
CCAGGTCCTCCGCGTTGCTCAGGGCGAACGGAGTCAACTTTGGTAGCTGCCTTCCCAA
AAAGGGTGCATGCCCAGGCTTTGAGTTGCACTCGCACCGTAGTGGCATCAGAAGGTG
ACCGTGCCCGGTCTGGGCGTTAGGATACAGCGCCTGCATGAAAGCCTTGATCTGCTTA
AAAGCCACCTGAGCCTTTGCGCCTTCAGAGAAGAACATGCCGCAAGACTTGCCGGAA
AACTGATTGGCCGGACAGGCCGCGTCATGCACGCAGCACCTTGCGTCGGTGTTGGAG
ATCTGCACCACATTTCGGCCCCACCGGTTCTTCACGATCTTGGCCTTGCTAGACTGCT
CCTTCAGCGCGCGCTGCCCGTTTTCGCTCGTCACATCCATTTCAATCACGTGCTCCTTA
TTTATCATAATGCTCCCGTGTAGACACTTAAGCTCGCCTTCGATCTCAGCGCAGCGGT
GCAGCCACAACGCGCAGCCCGTGGGCTCGTGGTGCTTGTAGGTTACCTCTGCAAACG
ACTGCAGGTACGCCTGCAGGAATCGCCCCATCATCGTCACAAAGGTCTTGTTGCTGGT
GAAGGTCAGCTGCAACCCGCGGTGCTCCTCGTTTAGCCAGGTCTTGCATACGGCCGCC
AGAGCTTCCACTTGGTCAGGCAGTAGCTTGAAGTTTGCCTTTAGATCGTTATCCACGT
GGTACTTGTCCATCAACGCGCGCGCAGCCTCCATGCCCTTCTCCCACGCAGACACGAT
CGGCAGGCTCAGCGGGTTTATCACCGTGCTTTCACTTTCCGCTTCACTGGACTCTTCCT
TTTCCTCTTGCGTCCGCATACCCCGCGCCACTGGGTCGTCTTCATTCAGCCGCCGCAC
CGTGCGCTTACCTCCCTTGCCGTGCTTGATTAGCACCGGTGGGTTGCTGAAACCCACC
ATTTGTAGCGCCACATCTTCTCTTTCTTCCTCGCTGTCCACGATCACCTCTGGGGATGG
CGGGCGCTCGGGCTTGGGAGAGGGGCGCTTCTTTTTCTTTTTGGACGCAATGGCCAAA
TCCGCCGTCGAGGTCGATGGCCGCGGGCTGGGTGTGCGCGGCACCAGCGCATCTTGT
GACGAGTCTTCTTCGTCCTCGGACTCGAGACGCCGCCTCAGCCGCTTTTTTGGGGGCG
CGCGCTTGTCGTCATCGTCTTTGTAGTCGGGAGGCGGCGGCGACGGCGACGGGGACG
ACACGTCCTCCATGGTTGGTGGACGTCGCGCCGCACCGCGTCCGCGCTCGGGGGTGG
TTTCGCGCTGCTCCTCTTCCCGACTGGCCATGGTGGCCGAGGATAACTTCGTATATGG
TTTCTTATACGAAGTTATGATCCAGACATGATAAGATACATTGATGAGTTTGGACAAA
CCACAACTAGAATGCAGTGAAAAAAATGCTTTATTTGTGAAATTTGTGATGCTATTGC
TTTATTTGTAACCATTATAAGCTGCAATAAACAAGTTAACAACAACAATTGCATTCAT
TTTATGTTTCAGGTTCAGGGGGAGGTGTGGGAGGTTTTTTAAAGCAAGTAAAACCTCT
ACAAATGTGGTATGGCTGATTATGATCCTCTAGAGTCGCAGATCTGCTACGTATCAAG
CTGTGGCAGGGAAACCCTCTGCCTCCCCCGTGATGTAATACTTTTGCAAGGAATGCGA
TGAAGTAGAGCCCGCAGTGGCCAAGTGGCTTTGGTCCGTCTCCTCCACGGATGCCCCT
CCACGGCTAGTGGGCGCATGTAGGCGGTGGGCGTCCGCCGCCTCCAGCAGCAGGTCA
TAGAGGGGCACCACGTTCTTGCACTTCATGCTGTACAGATGCTCCATGCCTTTGTTAC
TCATGTGTCGGATGTGGGAGAGGATGAGGAGGAGCTGGGCCAGCCGCTGGTGCTGCT
GCTGCAGGGTCAGGCCTGCCTTGGCCATCAGGTGGATCAAAGTGTCTGTGATCTTGTC
CAGGACTCGGTGGATATGGTCCTTCTCTTCCAGAGACTTCAGGGTGCTGGACAGAAA
TGTGTACACTCCAGAATTAAGCAAAATAATAGATTTGAGGCACACAAACTCCTCTCC
CTGCAGATTCATCATGCGGAACCGAGATGATGTAGCCAGCAGCATGTCGAAGATCTC
CACCATGCCCTCTACACATTTTCCCTGGTTCCTGTCCAAGAGCAAGTTAGGAGCAAAC
AGTAGCTTCACTGGGTGCTCCATGGAGCGCCAGACGAGACCAATCATCAGGATCTCT
AGCCAGGCACATTCTAGAAGGTGGACCTGATCATGGAGGGTCAAATCCACAAAGCCT
GGCACCCTCTTCGCCCAGTTGATCATGTGAACCAGCTCCCTGTCTGCCAGGTTGGTCA
GTAAGCCCATCATCGAAGCTTCACTGAAGGGTCTGGTAGGATCATACTCGGAATAGA
GTATGGGGGGCTCAGCATCCAACAAGGCACTGACCATCTGGTCGGCCGTCAGGGACA
AGGCCAGGCTGTTCTTCTTAGAGCGTTTGATCATGAGCGGGCTTGGCCAAAGGTTGGC
AGCTCTCATGTCTCCAGCAGATGGCTCGAGATCGCCATCTTCCAGCAGGCGCACCATT
GCCCCTGTTTCACTATCCAGGTTACGGATATAGTTCATGACAATATTTACATTGGTCC
AGCCACCAGCTTGCATGATCTCCGGTATTGAAACTCCAGCGCGGGCCATATCTCGCGC
GGCTCCGACACGGGCACTGTGTCCAGACCAGGCCAGGTATCTCTGACCAGAGTCATC
CTAAAATACACAAACAATTAGAATCAGTAGTTTAACACATTATACACTTAAAAATTTT
ATATTTACCTTAGCGCCGTAAATCAATCGATGAGTTGCTTCAAAAATCCCTTCCAGGG
CGCGAGTTGATAGCTGGCTGGTGGCAGATGGCGCGGCAACACCATTTTTTCTGACCC
GGCAAAACAGGTAGTTATTCGGATCATCAGCTACACCAGAGACGGAAATCCATCGCT
CGACCAGTTTAGTGACTCCCAGGCTAAGTGCCTTCTCTACACCTGCGGTGCTAACCAG
CGTTTTCGTTCTGCCAATATGGATTAACATTCTCCCACCGTCAGTACGTGAGATATCTT
TAACCCTGATCCTGGCAATTTCGGCTATACGTAACAGGGTGTTATAAGCAATCCCCAG
AAATGCCAGATTACGTATATCCTGGCAGCGATCGCTATTTTCCATGAGTGAACGGACT
TGGTCGAAATCAGTGCGTTCGAACGCTAGAGCCTGTTTTGCACGTTCACCGGCATCAA
CGTTTTCTTTTCGGATCCGCCGCATAACCAGTGAAACAGCATTGCTGTCACTTGGTCG
TGGCAGCCCGGACCGACGATGAAGCATGTTTAGCTGGCCCAAATGTTGCTGGATAGT
TTTTACTGCCAGACCGCGCGCTTGAAGATATAGAAGATAATCGCGAACATCTTCAGG
TTCTGCGGGAAACCATTTCCGGTTATTCAACTTGCACCATGCCGCCCACGACCGGCAA
ACGGACAGAAGCATTTTCCAGGTATGCTCAGAAAACGCCTGGCGATCCCTGAACATG
TCCATCAGGTTCTTGCGAACCTCATCACTCGTTGCATCGACCGGTAATGCAGGCAAAT
TTTGGTGTACGGTCAGTAAATTGGACATGGTGGCTACGTAATAACTTCGTATATGGTT
TCTTATACGAAGTTATGCGGCCGCTTTACGAGGGTAGGAAGTGGTACGGAAAGTTGG
TATAAGACAAAAGTGTTGTGGAATTGCTCCAGGCGATCTGACGGTTCACTAAACGAG
CTCTGCTTTTATAGGCGCCCACCGTACACGCCTAAAGCTTATACGTTCTCTATCACTG
ATAGGGAGTAAACTGGATATACGTTCTCTATCACTGATAGGGAGTAAACTGTAGATA
CGTTCTCTATCACTGATAGGGAGTAAACTGGTCATACGTTCTCTATCACTGATAGGGA
GTAAACTCCTTATACGTTCTCTATCACTGATAGGGAGTAAAGTCTGCATACGTTCTCT
ATCACTGATAGGGAGTAAACTCTTCATACGTTCTCTATCACTGATAGGGAGTAAACTC
GCGGCCGCAGAGAAATGTTCTGGCACCTGCACTTGCACTGGGGACAGCCTATTTTGCT
AGTTTGTTTTGTTTCGTTTTGTTTTGATGGAGAGCGTATGTTAGTACTATCGATTCACA
CAAAAAACCAACACACAGATGTAATGAAAATAAAGATATTTTATTGGATCTGCGATC
GCTCCGGTGCCCGTCAGTGGGCAGAGCGCACATCGCCCACAGTCCCCGAGAAGTTGG
GGGGAGGGGTCGGCAATTGAACGGGTGCCTAGAGAAGGTGGCGCGGGGTAAACTGG
GAAAGTGATGTCGTGTACTGGCTCCGCCTTTTTCCCGAGGGTGGGGGAGAACCGTAT
GTAAGTGCAGTAGTCGCCGTGAACGTTCTTTTTCGCAACGGGTTTGCCGCCAGAACAC
AGCTGAAGCTTCGAGGGGCTCGCATCTCTCCTTCACGCGCCCGCCGCCCTACCTGAGG
CCGCCATCCACGCCGGTTGAGTCGCGTTCTGCCGCCTCCCGCCTGTGGTGCCTCCTGA
ACTGCGTCCGCCGTCTAGGTAAGTTTAAAGCTCAGGTCGAGACCGGGCCTTTGTCCGG
CGCTCCCTTGGAGCCTACCTAGACTCAGCCGGCTCTCCACGCTTTGCCTGACCCTGCT
TGCTCAACTCTACGTCTTTGTTTCGTTTTCTGTTCTGCGCCGTTACAGATCCAAGCTGT
GACCGGCGCCTACGCTAGCGGATCCGCCGCCACCATGTCTAGACTGGACAAGAGCAA
AGTCATAAACTCTGCTCTGGAATTACTCAATGGAGTCGGTATCGAAGGCCTGACGAC
AAGGAAACTCGCTCAAAAGCTGGGAGTTGAGCAGCCTACCCTGTACTGGCACGTGAA
GAACAAGCGGGCCCTGCTCGATGCCCTGCCAATCGAGATGCTGGACAGGCATCATAC
CCACTCCTGCCCCCTGGAAGGCGAGTCATGGCAAGACTTTCTGCGGAACAACGCCAA
GTCATACCGCTGTGCTCTTCTCTCACATCGCGACGGGGCTAAAGTGCATCTCGGCACC
CGCCCAACAGAGAAACAGTACGAAACCCTGGAAAATCAGCTCGCGTTCCTGTGTCAG
CAAGGCTTCTCCCTGGAGAACGCACTGTACGCTCTGTCCGCCGTGGGCCACTTTACAC
TGGGCTGCGTATTGGAGGAACAGGAGCATCAAGTAGCAAAAGAGGAAAGAGAGACA
CCTACCACCGATTCTATGCCCCCACTTCTGAAACAAGCAATTGAGCTGTTCGACCGGC
AGGGAGCCGAACCTGCCTTCCTTTTCGGCCTGGAACTAATCATATGTGGCCTGGAGA
AACAGCTAAAGTGCGAAAGCGGCGGGCCGACCGACGCCCTTGACGATTTTGACTTAG
ACATGCTCCCAGCCGATGCCCTTGACGACTTTGACCTTGATATGCTGCCTGCTGACGC
TCTTGACGATTTTGACCTTGACATGCTCCCCGGGTGAACCGGTCGCTGATCAGCCTCG
ACTGTGCCTTCTAGTTGCCAGCCATCTGTTGTTTGCCCCTCCCCCGTGCCTTCCTTGAC
CCTGGAAGGTGCCACTCCCACTGTCCTTTCCTAATAAAATGAGGAAATTGCATCGCAT
TGTCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGGCTTCT
GAGGCGGAAAGAACCAGCTGGGGCTCGACTAGAGCTTGCGGAACCCTTAGAGGGCC
TATTTCCCATGATTCCTTCATATTTGCATATACGATACAAGGCTGTTAGAGAGATAAT
TAGAATTAATTTGACTGTAAACACAAAGATATTAGTACAAAATAATAACTTCGTATA
ATGTATGCTATACGAAGTTATCAGACATGATAAGATACATTGATGAGTTTGGACAAA
CCACAACTAGAATGCAGTGAAAAAAATGCTTTATTTGTGAAATTTGTGATGCTATTGC
TTTATTTGTAACCATTATAAGCTGCAATAAACAAGGTACCTCAAGCGCCGGGTTTTCG
CGTCATGCACCACGTCCGTGGGCCCTCGGGTACTTCAACGTCAGCAGTAACTGTAAAT
CCGAGCCGTTCATAGAAGGGCAAATTCCTTGGCGCTGACGTTTCAAGAAAGGCTGGC
ACTCCGGCTCGTTCTGCGGCTTCTACTCCGGGCAATACCACCGCGGAACCAAGGCCCT
TTCCCTGATGATCGGGGCTAACGCCCACAGTAGCGAGGAACCAAGCTGGTTCTTTAG
GGCGGTGAGGGGCGAGGAGTCCTTCCATTTGTTGCTGAGCCGCGAGACGAGAGCCAC
TAAGCTCAGCCATTCGGGGACCAATTTCTGCAAATACAGCCCCGGCCTCAACGCTCTC
CGGAGTCGTCCACACTGCCACTGCAGCCCCGTCGTCGGCGACCCAAACTTTACCGAT
GTCCAATCCTACCCTGGTCAAAAAAAGTTCTTGCAATTCTGTAACCCGTTCAATATGT
CTATCAGGATCAACTGTGTGGCGTGTAGCGGGATAATCCGCGAAAGCGGCAGCCAAT
GTTCTCACGGCCCTAGGGACGTCGTCTCGAGTTGCCAGTCTGACAGTAGGTTTATATT
CTGTCATAGGTCCAGGGTTCTCCTCCACGTCTCCAGCCTGCTTCAGCAGGCTGAAGTT
AGTAGCTCCGGATCCTTTACCTCCATCACCAGCGCCACCAGTAGAGTATCTGGCCACA
GCCACCTCGTGCTGCTCGACGTAGGTCTCATCGTCGGCCTCCTTGATTCTTTCCAGTCT
GTGGTCCACGTTGTAGACGCCGGGCATCTTGAGGTTCGTAGCGGGTTTCTTGGATCTG
TATGTGGTCTCAAGGTTGCAGATCAGGTGGCCCCCGCCCACGAGCTTCAGGGCCATG
TCACATGCGCCTTCCAGGCCGCCGTCAGCGGGGTACATCGTCTCGGTGGAGGCCTCCC
AGCCGAGTGTTTTCTTCTGCATCACAGGGCCGTTGGCTGGGAAGTTCACCCCTCTAAC
CTTGACGTTGTAGATGAGGCAGCCGTCCTGGAGGCTGGTGTCCTGGGTAGCGGTCAG
CACGCCCCCGTCTTCGTATGTGGTGACTCTCTCCCATGTGAAGCCCTCAGGGAAGGAC
TGCTTAAAGAAGTCGGGGATGCCCGGAGGGTGCTTGATGAAGGTTCTGCTGCCGTAC
ATGAAGCTGGTAGCCAGGATGTCGAAGGCGAAGGGGAGAGGGCCGCCCTCGACGAC
CTTGATTCTCATGGTCTGGGTGCCCTCGTAGGGCTTGCCTTCGCCCTCGGATGTGCAC
TTGAAGTGGTGGTTGTTCACGGTGCCCTCCATGTACAGCTTCATGGGCATGTTCTCCT
TAATCAGCTCGCTCACGGTGGCGGCGAATTCCGAAAGGCCCGGAGATGAGGAAGAG
GAGAACAGCGCGGCAGACGTGCGCTTTTGAAGCGTGCAGAATGCCGGGCCTCCGGAG
GACCTTCGGGCGCCCGCCCCGCCCCTGAGCCCGCCCCTGAGCCCGCCCCCGGACCCA
CCCCTTCCCAGCCTCTGAGCCCAGAAAGCGAAGGAGCAAAGCTGCTATTGGCCGCTG
CCCCAAAGGCCTACCCGCTTCCATTGCTCAGCGGTGCTGTCCATCTGCACGAGACTAG
TGAGACGTGCTACTTCCATTTGTCACGTCCTGCACGACGCGAGCTGCGGGGCGGGGG
GGAACTTCCTGACTAGGGGAGGAGTAGAAGGTGGCGCGAAGGGGCCACCAAAGAAC
GGAGCCGGTTGGCGCCTACCGGTGGATGTGGAATGTGTGCGAGGCCAGAGGCCACTT
GTGTAGCGCCAAGTGCCCAGCGGGGCTGCTAAAGCGCATGCTCCAGACTGCCTTGGG
AAAAGCGCTCCCCTACCCATAACTTCGTATAATGTATGCTATACGAAGTTATTTTGCA
GTTTTAAAATTATGTTTTAAAATGGACTATCATATGCTTACCGTAACTTGAAAGTATT
TCGATTTCTTGGCTTTATATATCTTGTGGAAAGGACGAAACACCGGGCACTCTTCCGT
GATCTGGTGGATAAATTCGCAAGGGTATCATGGCGGACGACCGGGATTCGAACCCCG
GATCCGGCCGTCCGCCGTGATCCATGCGGTTACCGCCCGCGTGTCGAACCCAGGTGT
GCGACGTCAGACAACGGGGGAGCGCTCCTTTTTGGGCCCATTGGTATGGCTTTTTCCC
CGTATCCCCCCAGGTGTCTGCAGGCTCAAAGAGCAGCGAGAAGCGTTCAGAGGAAAG
CGATCCCGTGCCACCTTCCCCGTGCCCGGGCTGTCCCCGCACGCTGCCGGCTCGGGGA
TGCGGGGGGAGCGCCGGACCGGAGCGGAGCCCCGGGCGGCTCGCTGCTGCCCCCTAG
CGGGGGAGGGACGTAATTACATCCCTGGGGGCTTTGGGGGGGGGCTGTCCCTGATAT
CTATAACAAGAAAATATATATATAATAAGTTATCACGTAAGTAGAACATGAAATAAC
AATATAATTATCGTATGAGTTAAATCTTAAAAGTCACGTAAAAGATAATCATGCGTC
ATTTTGACTCACGCGGTCGTTATAGTTCAAAATCAGTGACACTTACCGCATTGACAAG
CACGCCTCACGGGAGCTCCAAGCGGCGACTGAGATGTCCTAAATGCACAGCGACGGA
TTCGCGCTATTTAGAAAGAGAGAGCAATATTTCAAGAATGCATGCGTCAATTTTACGC
AGACTATCTTTCTAGGGTTAATCTAGCTGCATCAGGATCATATCGTCGGGTCTTTTTTC
CGGCTCAGTCATCGCCCAAGCTGGCGCTATCTGGGCATCGGGGAGGAAGAAGCCCGT
GCCTTTTCCCGCGAGGTTGAAGCGGCATGGAAAGAGTTTGCCGAGGATGACTGCTGC
TGCATTGACGTTGAGCGAAAACGCACGTTTACCATGATGATTCGGGAAGGTGTGGCC
ATGCACGCCTTTAACGGTGAACTGTTCGTTCAGGCCACCTGGGATACCAGTTCGTCGC
GGCTTTTCCGGACACAGTTCCGGATGGTCAGCCCGAAGCGCATCAGCAACCCGAACA
ATACCGGCGACAGCCGGAACTGCCGTGCCGGTGTGCAGATTAATGACAGCGGTGCGG
CGCTGGGATATTACGTCAGCGAGGACGGGTATCCTGGCTGGATGCCGCAGAAATGGA
CATGGATACCCCGTGAGTTACCCGGCGGGCGCGCTTGGCGTAATCATGGTCATAGCT
GTTTCCTGTGTGAAATTGTTATCCGCTCACAATTCCACACAACATACGAGCCGGAAGC
ATAAAGTGTAAAGCCTGGGGTGCCTAATGAGTGAGCTAACTCACATTAATTGCGTTG
CGCTCACTGCCCGCTTTCCAGTCGGGAAACCTGTCGTGCCAGCTGCATTAATGAATCG
GCCAACGCGCGGGGAGAGGCGGTTTGCGTATTGGGCGCTCTTCCGCTTCCTCGCTCAC
TGACTCGCTGCGCTCGGTCGTTCGGCTGCGGCGAGCGGTATCAGCTCACTCAAAGGC
GGTAATACGGTTATCCACAGAATCAGGGGATAACGCAGGAAAGAACATGTGAGCAA
AAGGCCAGCAAAAGGCCAGGAACCGTAAAAAGGCCGCGTTGCTGGCGTTTTTCCATA
GGCTCCGCCCCCCTGACGAGCATCACAAAAATCGACGCTCAAGTCAGAGGTGGCGAA
ACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAGC
GTGGCGCTTTCTCATAGCTCACGCTGTAGGTATCTCAGTTCGGTGTAGGTCGTTCGCT
CCAAGCTGGGCTGTGTGCACGAACCCCCCGTTCAGCCCGACCGCTGCGCCTTATCCGG
TAACTATCGTCTTGAGTCCAACCCGGTAAGACACGACTTATCGCCACTGGCAGCAGC
CACTGGTAACAGGATTAGCAGAGCGAGGTATGTAGGCGGTGCTACAGAGTTCTTGAA
GTGGTGGCCTAACTACGGCTACACTAGAAGGACAGTATTTGGTATCTGCGCTCTGCTG
AAGCCAGTTACCTTCGGAAAAAGAGTTGGTAGCTCTTGATCCGGCAAACAAACCACC
GCTGGTAGCGGTGGTTTTTTTGTTTGCAAGCAGCAGATTACGCGCAGAAAAAAAGGA
TCTCAAGAAGATCCTTTGATCTTTTCTACGGGGTCTGACGCTCAGTGGAACGAAAACT
CACGTTAAGGGATTTTGGTCATGAGATTATCAAAAAGGATCTTCACCTAGATCCTTTT
AAATTAAAAATGAAGTTTTAAATCAATCTAAAGTATATATGAGTAAACTTGGTCTGA
CAGTTACCAATGCTTAATCAGTGAGGCACCTATCTCAGCGATCTGTCTATTTCGTTCA
TCCATAGTTGCCTGACTCCCCGTCGTGTAGATAACTACGATACGGGAGGGCTTACCAT
CTGGCCCCAGTGCTGCAATGATACCGCGAGACCCACGCTCACCGGCTCCAGATTTATC
AGCAATAAACCAGCCAGCCGGAAGGGCCGAGCGCAGAAGTGGTCCTGCAACTTTATC
CGCCTCCATCCAGTCTATTAATTGTTGCCGGGAAGCTAGAGTAAGTAGTTCGCCAGTT
AATAGTTTGCGCAACGTTGTTGCCATTGCTACAGGCATCGTGGTGTCACGCTCGTCGT
TTGGTATGGCTTCATTCAGCTCCGGTTCCCAACGATCAAGGCGAGTTACATGATCCCC
CATGTTGTGCAAAAAAGCGGTTAGCTCCTTCGGTCCTCCGATCGTTGTCAGAAGTAAG
TTGGCCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCTGAGA
ATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCC
AACTGATCTTCAGCATCTTTTACTTTCACCAGCGTTTCTGGGTGAGCAAAAACAGGAA
GGCAAAATGCCGCAAAAAAGGGAATAAGGGCGACACGGAAATGTTGAATACTCAT
SEQ ID NO: 10 (STXC0110)
ACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCAC
ATTTCCCCGAAAAGTGCCACCTAAATTGTAAGCGTTAATATTTTGTTAAAATTCGCGT
TAAATTTTTGTTAAATCAGCTCATTTTTTAACCAATAGGCCGAAATCGGCAAAATCCC
TTATAAATCAAAAGAATAGACCGAGATAGGGTTGAGTGTTGTTCCAGTTTGGAACAA
GAGTCCACTATTAAAGAACGTGGACTCCAACGTCAAAGGGCGAAAAACCGTCTATCA
GGGCGATGGCCCACTACGTGAACCATCACCCTAATCAAGTTTTTTGGGGTCGAGGTG
CCGTAAAGCACTAAATCGGAACCCTAAAGGGAGCCCCCGATTTAGAGCTTGACGGGG
AAAGCCGGCGAACGTGGCGAGAAAGGAAGGGAAGAAAGCGAAAGGAGCGGGCGCT
AGGGCGCTGGCAAGTGTAGCGGTCACGCTGCGCGTAACCACCACACCCGCCGCGCTT
AATGCGCCGCTACAGGGCGCGTCCCATTCGCCATTCAGGCTGCGCAACTGTTGGGAA
GGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACG
ACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACG
CTGCAGAACACGCAGCTAGATTAACCCTAGAAAGATAATCATATTGTGACGTACGTT
AAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATA
AATTCAACAAACAATTTATTTATGTTTATTTATTTATTAAAAAAAACAAAAACTCAAA
ATTTCTTCTATAAAGTAACAAAACTTTTATGAGGGACAGCCCCCCCCCAAAGCCCCCA
GGGATGTAATTACGTCCCTCCCCCGCTAGGGGGCAGCAGCGAGCCGCCCGGGGCTCC
GCTCCGGTCCGGCGCTCCCCCCGCATCCCCGAGCCGGCAGCGTGCGGGGACAGCCCG
GGCACGGGGAAGGTGGCACGGGATCGCTTTCCTCTGAACGCTTCTCGCTGCTCTTTGA
GCCTGCAGACACCTGGGGGGATACGGGGAAAAGGCCTCCACGGCCACTAGTCCATAG
AGCCCACCGCATCCCCAGCATGCCTGCTATTGTCTTCCCAATCCTCCCCCTTGCTGTCC
TGCCCCACCCCACCCCCTAGAATAGAATGACACCTACTCAGACAATGCGATGCAATT
TCCTCATTTTATTAGGAAAGGACAGTGGGAGTGGCACCTTCCAGGGTCAAGGAAGGC
ACGGGGGAGGGGCAAACAACAGATGGCTGGCAACTAGAAGGCACAGCTACATGGGG
GTAGAGTCATAATCGTGCATCAGGATAGGGCGGTGGTGCTGCAGCAGCGCGCGAATA
AACTGCTGCCGCCGCCGCTCCGTCCTGCAGGAATACAACATGGCAGTGGTCTCCTCA
GCGATGATTCGCACCGCCCGCAGCATGAGACGCCTTGTCCTCCGGGCACAGCAGCGC
ACCCTGATCTCACTTAAATCAGCACAGTAACTGCAGCACAGCACCACAATATTGTTCA
AAATCCCACAGTGCAAGGCGCTGTATCCAAAGCTCATGGCGGGGACCACAGAACCCA
CGTGGCCATCATACCACAAGCGCAGGTAGATTAAGTGGCGACCCCTCATAAACACGC
TGGACATAAACATTACCTCTTTTGGCATGTTGTAATTCACCACCTCCCGGTACCATAT
AAACCTCTGATTAAACATGGCGCCATCCACCACCATCCTAAACCAGCTGGCCAAAAC
CTGCCCGCCGGCTATGCACTGCAGGGAACCGGGACTGGAACAATGACAGTGGAGAG
CCCAGGACTCGTAACCATGGATCATCATGCTCGTCATGATATCAATGTTGGCACAACA
CAGGCACACGTGCATACACTTCCTCAGGATTACAAGCTCCTCCCGCGTCAGAACCAT
ATCCCAGGGAACAACCCATTCCTGAATCAGCGTAAATCCCACACTGCAGGGAAGACC
TCGCACGTAACTCACGTTGTGCATTGTCAAAGTGTTACATTCGGGCAGCAGCGGATG
ATCCTCCAGTATGGTAGCGCGGGTCTCTGTCTCAAAAGGAGGTAGGCGATCCCTACT
GTACGGAGTGCGCCGAGACAACCGAGATCGTGTTGGTCGTAGTGTCATGCCAAATGG
AACGCCGGACGTAGTCATGGTTGTGGCCATATTATCATCGTGTTTTTCAAAGGAAAAC
CACGTCCCCGTGGTTCGGGGGGCCTAGACGTTTTTTTAACCTCGACTAAACACATGTA
AAGCATGTGCACCGAGGCCCCAGATCAGATCCCATACAATGGGGTACCTTCTGGGCA
TCCTTCAGCCCCTTGTTGAATACGCTTGAGGAGAGCCATTTGACTCTTTCCACAACTA
TCCAACTCACAACGTGGCACTGGGGTTGTGCCGCCTTTGCAGGTGTATCTTATACACG
TGGCTTTTGGCCGCAGAGGCACCTGTCGCCAGGTGGGGGGTTCCGCTGCCTGCAAAG
GGTCGCTACAGACGTTGTTTGTCTTCAAGAAGCTTCCAGAGGAACTGCTTCCTTCACG
ACATTCAACAGACCTTGCATTCCTTTGGCGAGAGGGGAAAGACCCCTAGGAATGCTC
GTCAAGAAGACAGGGCCAGGTTTCCGGGCCCTCACATTGCCAAAAGACGGCAATATG
GTGGAAAATAACATATAGACAAACGCACACCGGCCTTATTCCAAGCGGCTTCGGCCA
GTAACGTTAGGGGGGGGGGAGGGAGAGGGGCTTAAAAATCAAAGGGGTTCTGCCGC
GCATCACTATGCGCCACTGGCAGGGACACGTTGCGATACTGGTGTTTAGTGCTCCACT
TAAACTCAGGCACAACCATCCGCGGCAGCTCGGTGAAGTTTTCACTCCACAGGCTGC
GCACCATCACCAACGCGTTTAGCAGGTCGGGCGCCGATATCTTGAAGTCGCAGTTGG
GGCCTCCGCCCTGCGCGCGCGAGTTGCGATACACAGGGTTGCAGCACTGGAACACTA
TCAGCGCCGGGTGGTGCACGCTGGCCAGCACGCTCTTGTCGGAGATCAGATCCGCGT
CCAGGTCCTCCGCGTTGCTCAGGGCGAACGGAGTCAACTTTGGTAGCTGCCTTCCCAA
AAAGGGTGCATGCCCAGGCTTTGAGTTGCACTCGCACCGTAGTGGCATCAGAAGGTG
ACCGTGCCCGGTCTGGGCGTTAGGATACAGCGCCTGCATGAAAGCCTTGATCTGCTTA
AAAGCCACCTGAGCCTTTGCGCCTTCAGAGAAGAACATGCCGCAAGACTTGCCGGAA
AACTGATTGGCCGGACAGGCCGCGTCATGCACGCAGCACCTTGCGTCGGTGTTGGAG
ATCTGCACCACATTTCGGCCCCACCGGTTCTTCACGATCTTGGCCTTGCTAGACTGCT
CCTTCAGCGCGCGCTGCCCGTTTTCGCTCGTCACATCCATTTCAATCACGTGCTCCTTA
TTTATCATAATGCTCCCGTGTAGACACTTAAGCTCGCCTTCGATCTCAGCGCAGCGGT
GCAGCCACAACGCGCAGCCCGTGGGCTCGTGGTGCTTGTAGGTTACCTCTGCAAACG
ACTGCAGGTACGCCTGCAGGAATCGCCCCATCATCGTCACAAAGGTCTTGTTGCTGGT
GAAGGTCAGCTGCAACCCGCGGTGCTCCTCGTTTAGCCAGGTCTTGCATACGGCCGCC
AGAGCTTCCACTTGGTCAGGCAGTAGCTTGAAGTTTGCCTTTAGATCGTTATCCACGT
GGTACTTGTCCATCAACGCGCGCGCAGCCTCCATGCCCTTCTCCCACGCAGACACGAT
CGGCAGGCTCAGCGGGTTTATCACCGTGCTTTCACTTTCCGCTTCACTGGACTCTTCCT
TTTCCTCTTGCGTCCGCATACCCCGCGCCACTGGGTCGTCTTCATTCAGCCGCCGCAC
CGTGCGCTTACCTCCCTTGCCGTGCTTGATTAGCACCGGTGGGTTGCTGAAACCCACC
ATTTGTAGCGCCACATCTTCTCTTTCTTCCTCGCTGTCCACGATCACCTCTGGGGATGG
CGGGCGCTCGGGCTTGGGAGAGGGGCGCTTCTTTTTCTTTTTGGACGCAATGGCCAAA
TCCGCCGTCGAGGTCGATGGCCGCGGGCTGGGTGTGCGCGGCACCAGCGCATCTTGT
GACGAGTCTTCTTCGTCCTCGGACTCGAGACGCCGCCTCAGCCGCTTTTTTGGGGGCG
CGCGCTTGTCGTCATCGTCTTTGTAGTCGGGAGGCGGCGGCGACGGCGACGGGGACG
ACACGTCCTCCATGGTTGGTGGACGTCGCGCCGCACCGCGTCCGCGCTCGGGGGTGG
TTTCGCGCTGCTCCTCTTCCCGACTGGCCATGGTGGCCGAGGATAACTTCGTATATGG
TTTCTTATACGAAGTTATGATCCAGACATGATAAGATACATTGATGAGTTTGGACAAA
CCACAACTAGAATGCAGTGAAAAAAATGCTTTATTTGTGAAATTTGTGATGCTATTGC
TTTATTTGTAACCATTATAAGCTGCAATAAACAAGTTAACAACAACAATTGCATTCAT
TTTATGTTTCAGGTTCAGGGGGAGGTGTGGGAGGTTTTTTAAAGCAAGTAAAACCTCT
ACAAATGTGGTATGGCTGATTATGATCCTCTAGAGTCGCAGATCTGCTACGTATCAAG
CTGTGGCAGGGAAACCCTCTGCCTCCCCCGTGATGTAATACTTTTGCAAGGAATGCGA
TGAAGTAGAGCCCGCAGTGGCCAAGTGGCTTTGGTCCGTCTCCTCCACGGATGCCCCT
CCACGGCTAGTGGGCGCATGTAGGCGGTGGGCGTCCGCCGCCTCCAGCAGCAGGTCA
TAGAGGGGCACCACGTTCTTGCACTTCATGCTGTACAGATGCTCCATGCCTTTGTTAC
TCATGTGTCGGATGTGGGAGAGGATGAGGAGGAGCTGGGCCAGCCGCTGGTGCTGCT
GCTGCAGGGTCAGGCCTGCCTTGGCCATCAGGTGGATCAAAGTGTCTGTGATCTTGTC
CAGGACTCGGTGGATATGGTCCTTCTCTTCCAGAGACTTCAGGGTGCTGGACAGAAA
TGTGTACACTCCAGAATTAAGCAAAATAATAGATTTGAGGCACACAAACTCCTCTCC
CTGCAGATTCATCATGCGGAACCGAGATGATGTAGCCAGCAGCATGTCGAAGATCTC
CACCATGCCCTCTACACATTTTCCCTGGTTCCTGTCCAAGAGCAAGTTAGGAGCAAAC
AGTAGCTTCACTGGGTGCTCCATGGAGCGCCAGACGAGACCAATCATCAGGATCTCT
AGCCAGGCACATTCTAGAAGGTGGACCTGATCATGGAGGGTCAAATCCACAAAGCCT
GGCACCCTCTTCGCCCAGTTGATCATGTGAACCAGCTCCCTGTCTGCCAGGTTGGTCA
GTAAGCCCATCATCGAAGCTTCACTGAAGGGTCTGGTAGGATCATACTCGGAATAGA
GTATGGGGGGCTCAGCATCCAACAAGGCACTGACCATCTGGTCGGCCGTCAGGGACA
AGGCCAGGCTGTTCTTCTTAGAGCGTTTGATCATGAGCGGGCTTGGCCAAAGGTTGGC
AGCTCTCATGTCTCCAGCAGATGGCTCGAGATCGCCATCTTCCAGCAGGCGCACCATT
GCCCCTGTTTCACTATCCAGGTTACGGATATAGTTCATGACAATATTTACATTGGTCC
AGCCACCAGCTTGCATGATCTCCGGTATTGAAACTCCAGCGCGGGCCATATCTCGCGC
GGCTCCGACACGGGCACTGTGTCCAGACCAGGCCAGGTATCTCTGACCAGAGTCATC
CTAAAATACACAAACAATTAGAATCAGTAGTTTAACACATTATACACTTAAAAATTTT
ATATTTACCTTAGCGCCGTAAATCAATCGATGAGTTGCTTCAAAAATCCCTTCCAGGG
CGCGAGTTGATAGCTGGCTGGTGGCAGATGGCGCGGCAACACCATTTTTTCTGACCC
GGCAAAACAGGTAGTTATTCGGATCATCAGCTACACCAGAGACGGAAATCCATCGCT
CGACCAGTTTAGTGACTCCCAGGCTAAGTGCCTTCTCTACACCTGCGGTGCTAACCAG
CGTTTTCGTTCTGCCAATATGGATTAACATTCTCCCACCGTCAGTACGTGAGATATCTT
TAACCCTGATCCTGGCAATTTCGGCTATACGTAACAGGGTGTTATAAGCAATCCCCAG
AAATGCCAGATTACGTATATCCTGGCAGCGATCGCTATTTTCCATGAGTGAACGGACT
TGGTCGAAATCAGTGCGTTCGAACGCTAGAGCCTGTTTTGCACGTTCACCGGCATCAA
CGTTTTCTTTTCGGATCCGCCGCATAACCAGTGAAACAGCATTGCTGTCACTTGGTCG
TGGCAGCCCGGACCGACGATGAAGCATGTTTAGCTGGCCCAAATGTTGCTGGATAGT
TTTTACTGCCAGACCGCGCGCTTGAAGATATAGAAGATAATCGCGAACATCTTCAGG
TTCTGCGGGAAACCATTTCCGGTTATTCAACTTGCACCATGCCGCCCACGACCGGCAA
ACGGACAGAAGCATTTTCCAGGTATGCTCAGAAAACGCCTGGCGATCCCTGAACATG
TCCATCAGGTTCTTGCGAACCTCATCACTCGTTGCATCGACCGGTAATGCAGGCAAAT
TTTGGTGTACGGTCAGTAAATTGGACATGGTGGCTACGTAATAACTTCGTATATGGTT
TCTTATACGAAGTTATGCGGCCGCTTTACGAGGGTAGGAAGTGGTACGGAAAGTTGG
TATAAGACAAAAGTGTTGTGGAATTGCTCCAGGCGATCTGACGGTTCACTAAACGAG
CTCTGCTTTTATAGGCGCCCACCGTACACGCCTAAAGCTTATACGTTCTCTATCACTG
ATAGGGAGTAAACTGGATATACGTTCTCTATCACTGATAGGGAGTAAACTGTAGATA
CGTTCTCTATCACTGATAGGGAGTAAACTGGTCATACGTTCTCTATCACTGATAGGGA
GTAAACTCCTTATACGTTCTCTATCACTGATAGGGAGTAAAGTCTGCATACGTTCTCT
ATCACTGATAGGGAGTAAACTCTTCATACGTTCTCTATCACTGATAGGGAGTAAACTC
GCGGCCGCAGAGAAATGTTCTGGCACCTGCACTTGCACTGGGGACAGCCTATTTTGCT
AGTTTGTTTTGTTTCGTTTTGTTTTGATGGAGAGCGTATGTTAGTACTATCGATTCACA
CAAAAAACCAACACACAGATGTAATGAAAATAAAGATATTTTATTGGATCTGCGATC
GCTCCGGTGCCCGTCAGTGGGCAGAGCGCACATCGCCCACAGTCCCCGAGAAGTTGG
GGGGAGGGGTCGGCAATTGAACGGGTGCCTAGAGAAGGTGGCGCGGGGTAAACTGG
GAAAGTGATGTCGTGTACTGGCTCCGCCTTTTTCCCGAGGGTGGGGGAGAACCGTAT
GTAAGTGCAGTAGTCGCCGTGAACGTTCTTTTTCGCAACGGGTTTGCCGCCAGAACAC
AGCTGAAGCTTCGAGGGGCTCGCATCTCTCCTTCACGCGCCCGCCGCCCTACCTGAGG
CCGCCATCCACGCCGGTTGAGTCGCGTTCTGCCGCCTCCCGCCTGTGGTGCCTCCTGA
ACTGCGTCCGCCGTCTAGGTAAGTTTAAAGCTCAGGTCGAGACCGGGCCTTTGTCCGG
CGCTCCCTTGGAGCCTACCTAGACTCAGCCGGCTCTCCACGCTTTGCCTGACCCTGCT
TGCTCAACTCTACGTCTTTGTTTCGTTTTCTGTTCTGCGCCGTTACAGATCCAAGCTGT
GACCGGCGCCTACGCTAGCGGATCCGCCGCCACCATGTCTAGACTGGACAAGAGCAA
AGTCATAAACTCTGCTCTGGAATTACTCAATGGAGTCGGTATCGAAGGCCTGACGAC
AAGGAAACTCGCTCAAAAGCTGGGAGTTGAGCAGCCTACCCTGTACTGGCACGTGAA
GAACAAGCGGGCCCTGCTCGATGCCCTGCCAATCGAGATGCTGGACAGGCATCATAC
CCACTCCTGCCCCCTGGAAGGCGAGTCATGGCAAGACTTTCTGCGGAACAACGCCAA
GTCATACCGCTGTGCTCTTCTCTCACATCGCGACGGGGCTAAAGTGCATCTCGGCACC
CGCCCAACAGAGAAACAGTACGAAACCCTGGAAAATCAGCTCGCGTTCCTGTGTCAG
CAAGGCTTCTCCCTGGAGAACGCACTGTACGCTCTGTCCGCCGTGGGCCACTTTACAC
TGGGCTGCGTATTGGAGGAACAGGAGCATCAAGTAGCAAAAGAGGAAAGAGAGACA
CCTACCACCGATTCTATGCCCCCACTTCTGAAACAAGCAATTGAGCTGTTCGACCGGC
AGGGAGCCGAACCTGCCTTCCTTTTCGGCCTGGAACTAATCATATGTGGCCTGGAGA
AACAGCTAAAGTGCGAAAGCGGCGGGCCGACCGACGCCCTTGACGATTTTGACTTAG
ACATGCTCCCAGCCGATGCCCTTGACGACTTTGACCTTGATATGCTGCCTGCTGACGC
TCTTGACGATTTTGACCTTGACATGCTCCCCGGGGGATCCGGAGCTACTAACTTCAGC
CTGCTGAAGCAGGCTGGAGACGTGGAGGAGAACCCTGGACCTATGACAGAATATAA
ACCTACTGTCAGACTGGCAACTCGAGACGACGTCCCTAGGGCCGTGAGAACATTGGC
TGCCGCTTTCGCGGATTATCCCGCTACACGCCACACAGTTGATCCTGATAGACATATT
GAACGGGTTACAGAATTGCAAGAACTTTTTTTGACCAGGGTAGGATTGGACATCGGT
AAAGTTTGGGTCGCCGACGACGGGGCTGCAGTGGCAGTGTGGACGACTCCGGAGAGC
GTTGAGGCCGGGGCTGTATTTGCAGAAATTGGTCCCCGAATGGCTGAGCTTAGTGGC
TCTCGTCTCGCGGCTCAGCAACAAATGGAAGGACTCCTCGCCCCTCACCGCCCTAAA
GAACCAGCTTGGTTCCTCGCTACTGTGGGCGTTAGCCCCGATCATCAGGGAAAGGGC
CTTGGTTCCGCGGTGGTATTGCCCGGAGTAGAAGCCGCAGAACGAGCCGGAGTGCCA
GCCTTTCTTGAAACGTCAGCGCCAAGGAATTTGCCCTTCTATGAACGGCTCGGATTTA
CAGTTACTGCTGACGTTGAAGTACCCGAGGGCCCACGGACGTGGTGCATGACGCGAA
AACCCGGCGCTTGAGTTTAAACCGCTGATCAGCCTCGACTGTGCCTTCTAGTTGCCAG
CCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAG
CAGGCATGCTGGGGATGCGGTGGGCTCTATGGCTTCTGAGGCGGAAAGAACCAGCTG
GGGCTCGACTAGAGCTTGCGGAACCCTTAGTTTAAACGGGCCCTTAATTAATCGATGT
AGGATGTTGCCCCTCCTGACGCGGTAGGAGAAGGGGAGGGTGCCCTGCATGTCTGCC
GCTGCTCTTGCTCTTGCCGCTGCTGAGGAGGGGGGCGCATCTGCCGCAGCACCGGAT
GCATCTGGGAAAAGCAAAAAAGGGGCTCGTCCCTGTTTCCGGAGGAATTTGCAAGCG
GGGTCTTGCATGACGGGGAGGCAAACCCCCGTTCGCCGCAGTCCGGCCGGCCCGAGA
CTCGAACCGGGGGTCCTGCGACTCAACCCTTGGAAAATAACCCTCCGGCTACAGGGA
GCGAGCCACTTAATGCTTTCGCTTTCCAGCCTAACCGCTTACGCCGCGCGCGGCCAGT
GGCCAAAAAAGCTAGCGCAGCAGCCGCCGCGCCTGGAAGGAAGCCAAAAGGAGCGC
TCCCCCGTTGTCTGACGTCGCACACCTGGGTTCGACACGCGGGCGGTAACCGCATGG
ATCACGGCGGACGGCCGGATCCGGGGTTCGAACCCCGGTCGTCCGCCATGATACCCT
TGCGAATTTATCCACCAGACCACGGAAGAGTGCCCGCTTACAGGCTCTCCTTTTGCAC
GGTCTAGAGCGTCAACGACTGCGCACGCCTCACCGGCCAGAGCGTCCCGACCATGGA
GCACTTTTTGCCGCTGCGCAACATCTGGAACCGCGTCCGCGACTTTCCGCGCGCCTCC
ACCACCGCCGCCGGCATCACCTGGATGTCCAGGTACATCTACGGATTACGGGGCCCA
TTGGTATGGCTTTTTCCCCGTATCCCCCCAGGTGTCTGCAGGCTCAAAGAGCAGCGAG
AAGCGTTCAGAGGAAAGCGATCCCGTGCCACCTTCCCCGTGCCCGGGCTGTCCCCGC
ACGCTGCCGGCTCGGGGATGCGGGGGGAGCGCCGGACCGGAGCGGAGCCCCGGGCG
GCTCGCTGCTGCCCCCTAGCGGGGGAGGGACGTAATTACATCCCTGGGGGCTTTGGG
GGGGGGCTGTCCCTGATATCTATAACAAGAAAATATATATATAATAAGTTATCACGT
AAGTAGAACATGAAATAACAATATAATTATCGTATGAGTTAAATCTTAAAAGTCACG
TAAAAGATAATCATGCGTCATTTTGACTCACGCGGTCGTTATAGTTCAAAATCAGTGA
CACTTACCGCATTGACAAGCACGCCTCACGGGAGCTCCAAGCGGCGACTGAGATGTC
CTAAATGCACAGCGACGGATTCGCGCTATTTAGAAAGAGAGAGCAATATTTCAAGAA
TGCATGCGTCAATTTTACGCAGACTATCTTTCTAGGGTTAATCTAGCTGCATCAGGAT
CATATCGTCGGGTCTTTTTTCCGGCTCAGTCATCGCCCAAGCTGGCGCTATCTGGGCA
TCGGGGAGGAAGAAGCCCGTGCCTTTTCCCGCGAGGTTGAAGCGGCATGGAAAGAGT
TTGCCGAGGATGACTGCTGCTGCATTGACGTTGAGCGAAAACGCACGTTTACCATGA
TGATTCGGGAAGGTGTGGCCATGCACGCCTTTAACGGTGAACTGTTCGTTCAGGCCAC
CTGGGATACCAGTTCGTCGCGGCTTTTCCGGACACAGTTCCGGATGGTCAGCCCGAA
GCGCATCAGCAACCCGAACAATACCGGCGACAGCCGGAACTGCCGTGCCGGTGTGCA
GATTAATGACAGCGGTGCGGCGCTGGGATATTACGTCAGCGAGGACGGGTATCCTGG
CTGGATGCCGCAGAAATGGACATGGATACCCCGTGAGTTACCCGGCGGGCGCGCTTG
GCGTAATCATGGTCATAGCTGTTTCCTGTGTGAAATTGTTATCCGCTCACAATTCCAC
ACAACATACGAGCCGGAAGCATAAAGTGTAAAGCCTGGGGTGCCTAATGAGTGAGCT
AACTCACATTAATTGCGTTGCGCTCACTGCCCGCTTTCCAGTCGGGAAACCTGTCGTG
CCAGCTGCATTAATGAATCGGCCAACGCGCGGGGAGAGGCGGTTTGCGTATTGGGCG
CTCTTCCGCTTCCTCGCTCACTGACTCGCTGCGCTCGGTCGTTCGGCTGCGGCGAGCG
GTATCAGCTCACTCAAAGGCGGTAATACGGTTATCCACAGAATCAGGGGATAACGCA
GGAAAGAACATGTGAGCAAAAGGCCAGCAAAAGGCCAGGAACCGTAAAAAGGCCGC
GTTGCTGGCGTTTTTCCATAGGCTCCGCCCCCCTGACGAGCATCACAAAAATCGACGC
TCAAGTCAGAGGTGGCGAAACCCGACAGGACTATAAAGATACCAGGCGTTTCCCCCT
GGAAGCTCCCTCGTGCGCTCTCCTGTTCCGACCCTGCCGCTTACCGGATACCTGTCCG
CCTTTCTCCCTTCGGGAAGCGTGGCGCTTTCTCATAGCTCACGCTGTAGGTATCTCAG
TTCGGTGTAGGTCGTTCGCTCCAAGCTGGGCTGTGTGCACGAACCCCCCGTTCAGCCC
GACCGCTGCGCCTTATCCGGTAACTATCGTCTTGAGTCCAACCCGGTAAGACACGACT
TATCGCCACTGGCAGCAGCCACTGGTAACAGGATTAGCAGAGCGAGGTATGTAGGCG
GTGCTACAGAGTTCTTGAAGTGGTGGCCTAACTACGGCTACACTAGAAGGACAGTAT
TTGGTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAGATT
ACGCGCAGAAAAAAAGGATCTCAAGAAGATCCTTTGATCTTTTCTACGGGGTCTGAC
GCTCAGTGGAACGAAAACTCACGTTAAGGGATTTTGGTCATGAGATTATCAAAAAGG
ATCTTCACCTAGATCCTTTTAAATTAAAAATGAAGTTTTAAATCAATCTAAAGTATAT
ATGAGTAAACTTGGTCTGACAGTTACCAATGCTTAATCAGTGAGGCACCTATCTCAGC
GATCTGTCTATTTCGTTCATCCATAGTTGCCTGACTCCCCGTCGTGTAGATAACTACG
ATACGGGAGGGCTTACCATCTGGCCCCAGTGCTGCAATGATACCGCGAGACCCACGC
TCACCGGCTCCAGATTTATCAGCAATAAACCAGCCAGCCGGAAGGGCCGAGCGCAGA
AGTGGTCCTGCAACTTTATCCGCCTCCATCCAGTCTATTAATTGTTGCCGGGAAGCTA
GAGTAAGTAGTTCGCCAGTTAATAGTTTGCGCAACGTTGTTGCCATTGCTACAGGCAT
CGTGGTGTCACGCTCGTCGTTTGGTATGGCTTCATTCAGCTCCGGTTCCCAACGATCA
AGGCGAGTTACATGATCCCCCATGTTGTGCAAAAAAGCGGTTAGCTCCTTCGGTCCTC
CGATCGTTGTCAGAAGTAAGTTGGCCGCAGTGTTATCACTCATGGTTATGGCAGCACT
GCATAATTCTCTTACTGTCATGCCATCCGTAAGATGCTTTTCTGTGACTGGTGAGTACT
CAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAA
ACGTTCTTCGGGGCGAAAACTCTCAAGGATCTTACCGCTGTTGAGATCCAGTTCGATG
TAACCCACTCGTGCACCCAACTGATCTTCAGCATCTTTTACTTTCACCAGCGTTTCTGG
GTGAGCAAAAACAGGAAGGCAAAATGCCGCAAAAAAGGGAATAAGGGCGACACGG
AAATGTTGAATACTCAT
SEQ ID NO: 11 (Helper construct, V1)
GCCTCCACGGCCACTAGTCCATAGAGCCCACCGCATCCCCAGCATGCCTG
CTATTGTCTTCCCAATCCTCCCCCTTGCTGTCCTGCCCCACCCCACCCCCTAGAATAGA
ATGACACCTACTCAGACAATGCGATGCAATTTCCTCATTTTATTAGGAAAGGACAGTG
GGAGTGGCACCTTCCAGGGTCAAGGAAGGCACGGGGGAGGGGCAAACAACAGATGG
CTGGCAACTAGAAGGCACAGCTACATGGGGGTAGAGTCATAATCGTGCATCAGGATA
GGGCGGTGGTGCTGCAGCAGCGCGCGAATAAACTGCTGCCGCCGCCGCTCCGTCCTG
CAGGAATACAACATGGCAGTGGTCTCCTCAGCGATGATTCGCACCGCCCGCAGCATG
AGACGCCTTGTCCTCCGGGCACAGCAGCGCACCCTGATCTCACTTAAATCAGCACAG
TAACTGCAGCACAGCACCACAATATTGTTCAAAATCCCACAGTGCAAGGCGCTGTAT
CCAAAGCTCATGGCGGGGACCACAGAACCCACGTGGCCATCATACCACAAGCGCAG
GTAGATTAAGTGGCGACCCCTCATAAACACGCTGGACATAAACATTACCTCTTTTGGC
ATGTTGTAATTCACCACCTCCCGGTACCATATAAACCTCTGATTAAACATGGCGCCAT
CCACCACCATCCTAAACCAGCTGGCCAAAACCTGCCCGCCGGCTATGCACTGCAGGG
AACCGGGACTGGAACAATGACAGTGGAGAGCCCAGGACTCGTAACCATGGATCATC
ATGCTCGTCATGATATCAATGTTGGCACAACACAGGCACACGTGCATACACTTCCTCA
GGATTACAAGCTCCTCCCGCGTCAGAACCATATCCCAGGGAACAACCCATTCCTGAA
TCAGCGTAAATCCCACACTGCAGGGAAGACCTCGCACGTAACTCACGTTGTGCATTG
TCAAAGTGTTACATTCGGGCAGCAGCGGATGATCCTCCAGTATGGTAGCGCGGGTCT
CTGTCTCAAAAGGAGGTAGGCGATCCCTACTGTACGGAGTGCGCCGAGACAACCGAG
ATCGTGTTGGTCGTAGTGTCATGCCAAATGGAACGCCGGACGTAGTCATGGTTGTGG
CCATATTATCATCGTGTTTTTCAAAGGAAAACCACGTCCCCGTGGTTCGGGGGGCCTA
GACGTTTTTTTAACCTCGACTAAACACATGTAAAGCATGTGCACCGAGGCCCCAGATC
AGATCCCATACAATGGGGTACCTTCTGGGCATCCTTCAGCCCCTTGTTGAATACGCTT
GAGGAGAGCCATTTGACTCTTTCCACAACTATCCAACTCACAACGTGGCACTGGGGTT
GTGCCGCCTTTGCAGGTGTATCTTATACACGTGGCTTTTGGCCGCAGAGGCACCTGTC
GCCAGGTGGGGGGTTCCGCTGCCTGCAAAGGGTCGCTACAGACGTTGTTTGTCTTCAA
GAAGCTTCCAGAGGAACTGCTTCCTTCACGACATTCAACAGACCTTGCATTCCTTTGG
CGAGAGGGGAAAGACCCCTAGGAATGCTCGTCAAGAAGACAGGGCCAGGTTTCCGG
GCCCTCACATTGCCAAAAGACGGCAATATGGTGGAAAATAACATATAGACAAACGCA
CACCGGCCTTATTCCAAGCGGCTTCGGCCAGTAACGTTAGGGGGGGGGGAGGGAGAG
GGGCTTAAAAATCAAAGGGGTTCTGCCGCGCATCACTATGCGCCACTGGCAGGGACA
CGTTGCGATACTGGTGTTTAGTGCTCCACTTAAACTCAGGCACAACCATCCGCGGCAG
CTCGGTGAAGTTTTCACTCCACAGGCTGCGCACCATCACCAACGCGTTTAGCAGGTCG
GGCGCCGATATCTTGAAGTCGCAGTTGGGGCCTCCGCCCTGCGCGCGCGAGTTGCGA
TACACAGGGTTGCAGCACTGGAACACTATCAGCGCCGGGTGGTGCACGCTGGCCAGC
ACGCTCTTGTCGGAGATCAGATCCGCGTCCAGGTCCTCCGCGTTGCTCAGGGCGAAC
GGAGTCAACTTTGGTAGCTGCCTTCCCAAAAAGGGTGCATGCCCAGGCTTTGAGTTGC
ACTCGCACCGTAGTGGCATCAGAAGGTGACCGTGCCCGGTCTGGGCGTTAGGATACA
GCGCCTGCATGAAAGCCTTGATCTGCTTAAAAGCCACCTGAGCCTTTGCGCCTTCAGA
GAAGAACATGCCGCAAGACTTGCCGGAAAACTGATTGGCCGGACAGGCCGCGTCATG
CACGCAGCACCTTGCGTCGGTGTTGGAGATCTGCACCACATTTCGGCCCCACCGGTTC
TTCACGATCTTGGCCTTGCTAGACTGCTCCTTCAGCGCGCGCTGCCCGTTTTCGCTCGT
CACATCCATTTCAATCACGTGCTCCTTATTTATCATAATGCTCCCGTGTAGACACTTAA
GCTCGCCTTCGATCTCAGCGCAGCGGTGCAGCCACAACGCGCAGCCCGTGGGCTCGT
GGTGCTTGTAGGTTACCTCTGCAAACGACTGCAGGTACGCCTGCAGGAATCGCCCCA
TCATCGTCACAAAGGTCTTGTTGCTGGTGAAGGTCAGCTGCAACCCGCGGTGCTCCTC
GTTTAGCCAGGTCTTGCATACGGCCGCCAGAGCTTCCACTTGGTCAGGCAGTAGCTTG
AAGTTTGCCTTTAGATCGTTATCCACGTGGTACTTGTCCATCAACGCGCGCGCAGCCT
CCATGCCCTTCTCCCACGCAGACACGATCGGCAGGCTCAGCGGGTTTATCACCGTGCT
TTCACTTTCCGCTTCACTGGACTCTTCCTTTTCCTCTTGCGTCCGCATACCCCGCGCCA
CTGGGTCGTCTTCATTCAGCCGCCGCACCGTGCGCTTACCTCCCTTGCCGTGCTTGATT
AGCACCGGTGGGTTGCTGAAACCCACCATTTGTAGCGCCACATCTTCTCTTTCTTCCT
CGCTGTCCACGATCACCTCTGGGGATGGCGGGCGCTCGGGCTTGGGAGAGGGGCGCT
TCTTTTTCTTTTTGGACGCAATGGCCAAATCCGCCGTCGAGGTCGATGGCCGCGGGCT
GGGTGTGCGCGGCACCAGCGCATCTTGTGACGAGTCTTCTTCGTCCTCGGACTCGAGA
CGCCGCCTCAGCCGCTTTTTTGGGGGCGCGCGCTTGTCGTCATCGTCTTTGTAGTCGG
GAGGCGGCGGCGACGGCGACGGGGACGACACGTCCTCCATGGTTGGTGGACGTCGC
GCCGCACCGCGTCCGCGCTCGGGGGTGGTTTCGCGCTGCTCCTCTTCCCGACTGGCCA
TGGTGGCCGAGGATAACTTCGTATATGGTTTCTTATACGAAGTTATGATCCAGACATG
ATAAGATACATTGATGAGTTTGGACAAACCACAACTAGAATGCAGTGAAAAAAATGC
TTTATTTGTGAAATTTGTGATGCTATTGCTTTATTTGTAACCATTATAAGCTGCAATAA
ACAAGTTAACAACAACAATTGCATTCATTTTATGTTTCAGGTTCAGGGGGAGGTGTGG
GAGGTTTTTTAAAGCAAGTAAAACCTCTACAAATGTGGTATGGCTGATTATGATCCTC
TAGAGTCGCAGATCTGCTACGTATCAAGCTGTGGCAGGGAAACCCTCTGCCTCCCCC
GTGATGTAATACTTTTGCAAGGAATGCGATGAAGTAGAGCCCGCAGTGGCCAAGTGG
CTTTGGTCCGTCTCCTCCACGGATGCCCCTCCACGGCTAGTGGGCGCATGTAGGCGGT
GGGCGTCCGCCGCCTCCAGCAGCAGGTCATAGAGGGGCACCACGTTCTTGCACTTCA
TGCTGTACAGATGCTCCATGCCTTTGTTACTCATGTGTCGGATGTGGGAGAGGATGAG
GAGGAGCTGGGCCAGCCGCTGGTGCTGCTGCTGCAGGGTCAGGCCTGCCTTGGCCAT
CAGGTGGATCAAAGTGTCTGTGATCTTGTCCAGGACTCGGTGGATATGGTCCTTCTCT
TCCAGAGACTTCAGGGTGCTGGACAGAAATGTGTACACTCCAGAATTAAGCAAAATA
ATAGATTTGAGGCACACAAACTCCTCTCCCTGCAGATTCATCATGCGGAACCGAGAT
GATGTAGCCAGCAGCATGTCGAAGATCTCCACCATGCCCTCTACACATTTTCCCTGGT
TCCTGTCCAAGAGCAAGTTAGGAGCAAACAGTAGCTTCACTGGGTGCTCCATGGAGC
GCCAGACGAGACCAATCATCAGGATCTCTAGCCAGGCACATTCTAGAAGGTGGACCT
GATCATGGAGGGTCAAATCCACAAAGCCTGGCACCCTCTTCGCCCAGTTGATCATGT
GAACCAGCTCCCTGTCTGCCAGGTTGGTCAGTAAGCCCATCATCGAAGCTTCACTGAA
GGGTCTGGTAGGATCATACTCGGAATAGAGTATGGGGGGCTCAGCATCCAACAAGGC
ACTGACCATCTGGTCGGCCGTCAGGGACAAGGCCAGGCTGTTCTTCTTAGAGCGTTTG
ATCATGAGCGGGCTTGGCCAAAGGTTGGCAGCTCTCATGTCTCCAGCAGATGGCTCG
AGATCGCCATCTTCCAGCAGGCGCACCATTGCCCCTGTTTCACTATCCAGGTTACGGA
TATAGTTCATGACAATATTTACATTGGTCCAGCCACCAGCTTGCATGATCTCCGGTAT
TGAAACTCCAGCGCGGGCCATATCTCGCGCGGCTCCGACACGGGCACTGTGTCCAGA
CCAGGCCAGGTATCTCTGACCAGAGTCATCCTAAAATACACAAACAATTAGAATCAG
TAGTTTAACACATTATACACTTAAAAATTTTATATTTACCTTAGCGCCGTAAATCAAT
CGATGAGTTGCTTCAAAAATCCCTTCCAGGGCGCGAGTTGATAGCTGGCTGGTGGCA
GATGGCGCGGCAACACCATTTTTTCTGACCCGGCAAAACAGGTAGTTATTCGGATCAT
CAGCTACACCAGAGACGGAAATCCATCGCTCGACCAGTTTAGTGACTCCCAGGCTAA
GTGCCTTCTCTACACCTGCGGTGCTAACCAGCGTTTTCGTTCTGCCAATATGGATTAA
CATTCTCCCACCGTCAGTACGTGAGATATCTTTAACCCTGATCCTGGCAATTTCGGCT
ATACGTAACAGGGTGTTATAAGCAATCCCCAGAAATGCCAGATTACGTATATCCTGG
CAGCGATCGCTATTTTCCATGAGTGAACGGACTTGGTCGAAATCAGTGCGTTCGAAC
GCTAGAGCCTGTTTTGCACGTTCACCGGCATCAACGTTTTCTTTTCGGATCCGCCGCA
TAACCAGTGAAACAGCATTGCTGTCACTTGGTCGTGGCAGCCCGGACCGACGATGAA
GCATGTTTAGCTGGCCCAAATGTTGCTGGATAGTTTTTACTGCCAGACCGCGCGCTTG
AAGATATAGAAGATAATCGCGAACATCTTCAGGTTCTGCGGGAAACCATTTCCGGTT
ATTCAACTTGCACCATGCCGCCCACGACCGGCAAACGGACAGAAGCATTTTCCAGGT
ATGCTCAGAAAACGCCTGGCGATCCCTGAACATGTCCATCAGGTTCTTGCGAACCTCA
TCACTCGTTGCATCGACCGGTAATGCAGGCAAATTTTGGTGTACGGTCAGTAAATTGG
ACATGGTGGCTACGTAATAACTTCGTATATGGTTTCTTATACGAAGTTATGCGGCCGC
TTTACGAGGGTAGGAAGTGGTACGGAAAGTTGGTATAAGACAAAAGTGTTGTGGAAT
TGCTCCAGGCGATCTGACGGTTCACTAAACGAGCTCTGCTTTTATAGGCGCCCACCGT
ACACGCCTAAAGCTTATACGTTCTCTATCACTGATAGGGAGTAAACTGGATATACGTT
CTCTATCACTGATAGGGAGTAAACTGTAGATACGTTCTCTATCACTGATAGGGAGTAA
ACTGGTCATACGTTCTCTATCACTGATAGGGAGTAAACTCCTTATACGTTCTCTATCA
CTGATAGGGAGTAAAGTCTGCATACGTTCTCTATCACTGATAGGGAGTAAACTCTTCA
TACGTTCTCTATCACTGATAGGGAGTAAACTCGCGGCCGCAGAGAAATGTTCTGGCA
CCTGCACTTGCACTGGGGACAGCCTATTTTGCTAGTTTGTTTTGTTTCGTTTTGTTTTG
ATGGAGAGCGTATGTTAGTACTATCGATTCACACAAAAAACCAACACACAGATGTAA
TGAAAATAAAGATATTTTATTGGATCTGCGATCGCTCCGGTGCCCGTCAGTGGGCAG
AGCGCACATCGCCCACAGTCCCCGAGAAGTTGGGGGGAGGGGTCGGCAATTGAACG
GGTGCCTAGAGAAGGTGGCGCGGGGTAAACTGGGAAAGTGATGTCGTGTACTGGCTC
CGCCTTTTTCCCGAGGGTGGGGGAGAACCGTATGTAAGTGCAGTAGTCGCCGTGAAC
GTTCTTTTTCGCAACGGGTTTGCCGCCAGAACACAGCTGAAGCTTCGAGGGGCTCGCA
TCTCTCCTTCACGCGCCCGCCGCCCTACCTGAGGCCGCCATCCACGCCGGTTGAGTCG
CGTTCTGCCGCCTCCCGCCTGTGGTGCCTCCTGAACTGCGTCCGCCGTCTAGGTAAGT
TTAAAGCTCAGGTCGAGACCGGGCCTTTGTCCGGCGCTCCCTTGGAGCCTACCTAGAC
TCAGCCGGCTCTCCACGCTTTGCCTGACCCTGCTTGCTCAACTCTACGTCTTTGTTTCG
TTTTCTGTTCTGCGCCGTTACAGATCCAAGCTGTGACCGGCGCCTACGCTAGCGGATC
CGCCGCCACCATGTCTAGACTGGACAAGAGCAAAGTCATAAACTCTGCTCTGGAATT
ACTCAATGGAGTCGGTATCGAAGGCCTGACGACAAGGAAACTCGCTCAAAAGCTGGG
AGTTGAGCAGCCTACCCTGTACTGGCACGTGAAGAACAAGCGGGCCCTGCTCGATGC
CCTGCCAATCGAGATGCTGGACAGGCATCATACCCACTCCTGCCCCCTGGAAGGCGA
GTCATGGCAAGACTTTCTGCGGAACAACGCCAAGTCATACCGCTGTGCTCTTCTCTCA
CATCGCGACGGGGCTAAAGTGCATCTCGGCACCCGCCCAACAGAGAAACAGTACGA
AACCCTGGAAAATCAGCTCGCGTTCCTGTGTCAGCAAGGCTTCTCCCTGGAGAACGC
ACTGTACGCTCTGTCCGCCGTGGGCCACTTTACACTGGGCTGCGTATTGGAGGAACAG
GAGCATCAAGTAGCAAAAGAGGAAAGAGAGACACCTACCACCGATTCTATGCCCCC
ACTTCTGAAACAAGCAATTGAGCTGTTCGACCGGCAGGGAGCCGAACCTGCCTTCCT
TTTCGGCCTGGAACTAATCATATGTGGCCTGGAGAAACAGCTAAAGTGCGAAAGCGG
CGGGCCGACCGACGCCCTTGACGATTTTGACTTAGACATGCTCCCAGCCGATGCCCTT
GACGACTTTGACCTTGATATGCTGCCTGCTGACGCTCTTGACGATTTTGACCTTGACA
TGCTCCCCGGGTGAACCGGTCGCTGATCAGCCTCGACTGTGCCTTCTAGTTGCCAGCC
ATCTGTTGTTTGCCCCTCCCCCGTGCCTTCCTTGACCCTGGAAGGTGCCACTCCCACTG
TCCTTTCCTAATAAAATGAGGAAATTGCATCGCATTGTCTGAGTAGGTGTCATTCTAT
TCTGGGGGGTGGGGTGGGGCAGGACAGCAAGGGGGAGGATTGGGAAGACAATAGCA
GGCATGCTGGGGATGCGGTGGGCTCTATGGCTTCTGAGGCGGAAAGAACCAGCTGGG
GCTCGACTAGAGCTTGCGGAACCCTTAGAGGGCCTATTTCCCATGATTCCTTCATATT
TGCATATACGATACAAGGCTGTTAGAGAGATAATTAGAATTAATTTGACTGTAAACA
CAAAGATATTAGTACAAAATAATAACTTCGTATAATGTATGCTATACGAAGTTATCA
GACATGATAAGATACATTGATGAGTTTGGACAAACCACAACTAGAATGCAGTGAAAA
AAATGCTTTATTTGTGAAATTTGTGATGCTATTGCTTTATTTGTAACCATTATAAGCTG
CAATAAACAAGGTACCTCAAGCGCCGGGTTTTCGCGTCATGCACCACGTCCGTGGGC
CCTCGGGTACTTCAACGTCAGCAGTAACTGTAAATCCGAGCCGTTCATAGAAGGGCA
AATTCCTTGGCGCTGACGTTTCAAGAAAGGCTGGCACTCCGGCTCGTTCTGCGGCTTC
TACTCCGGGCAATACCACCGCGGAACCAAGGCCCTTTCCCTGATGATCGGGGCTAAC
GCCCACAGTAGCGAGGAACCAAGCTGGTTCTTTAGGGCGGTGAGGGGCGAGGAGTCC
TTCCATTTGTTGCTGAGCCGCGAGACGAGAGCCACTAAGCTCAGCCATTCGGGGACC
AATTTCTGCAAATACAGCCCCGGCCTCAACGCTCTCCGGAGTCGTCCACACTGCCACT
GCAGCCCCGTCGTCGGCGACCCAAACTTTACCGATGTCCAATCCTACCCTGGTCAAAA
AAAGTTCTTGCAATTCTGTAACCCGTTCAATATGTCTATCAGGATCAACTGTGTGGCG
TGTAGCGGGATAATCCGCGAAAGCGGCAGCCAATGTTCTCACGGCCCTAGGGACGTC
GTCTCGAGTTGCCAGTCTGACAGTAGGTTTATATTCTGTCATAGGTCCAGGGTTCTCC
TCCACGTCTCCAGCCTGCTTCAGCAGGCTGAAGTTAGTAGCTCCGGATCCTTTACCTC
CATCACCAGCGCCACCAGTAGAGTATCTGGCCACAGCCACCTCGTGCTGCTCGACGT
AGGTCTCATCGTCGGCCTCCTTGATTCTTTCCAGTCTGTGGTCCACGTTGTAGACGCC
GGGCATCTTGAGGTTCGTAGCGGGTTTCTTGGATCTGTATGTGGTCTCAAGGTTGCAG
ATCAGGTGGCCCCCGCCCACGAGCTTCAGGGCCATGTCACATGCGCCTTCCAGGCCG
CCGTCAGCGGGGTACATCGTCTCGGTGGAGGCCTCCCAGCCGAGTGTTTTCTTCTGCA
TCACAGGGCCGTTGGCTGGGAAGTTCACCCCTCTAACCTTGACGTTGTAGATGAGGC
AGCCGTCCTGGAGGCTGGTGTCCTGGGTAGCGGTCAGCACGCCCCCGTCTTCGTATGT
GGTGACTCTCTCCCATGTGAAGCCCTCAGGGAAGGACTGCTTAAAGAAGTCGGGGAT
GCCCGGAGGGTGCTTGATGAAGGTTCTGCTGCCGTACATGAAGCTGGTAGCCAGGAT
GTCGAAGGCGAAGGGGAGAGGGCCGCCCTCGACGACCTTGATTCTCATGGTCTGGGT
GCCCTCGTAGGGCTTGCCTTCGCCCTCGGATGTGCACTTGAAGTGGTGGTTGTTCACG
GTGCCCTCCATGTACAGCTTCATGGGCATGTTCTCCTTAATCAGCTCGCTCACGGTGG
CGGCGAATTCCGAAAGGCCCGGAGATGAGGAAGAGGAGAACAGCGCGGCAGACGTG
CGCTTTTGAAGCGTGCAGAATGCCGGGCCTCCGGAGGACCTTCGGGCGCCCGCCCCG
CCCCTGAGCCCGCCCCTGAGCCCGCCCCCGGACCCACCCCTTCCCAGCCTCTGAGCCC
AGAAAGCGAAGGAGCAAAGCTGCTATTGGCCGCTGCCCCAAAGGCCTACCCGCTTCC
ATTGCTCAGCGGTGCTGTCCATCTGCACGAGACTAGTGAGACGTGCTACTTCCATTTG
TCACGTCCTGCACGACGCGAGCTGCGGGGGGGGGGGGAACTTCCTGACTAGGGGAGG
AGTAGAAGGTGGCGCGAAGGGGCCACCAAAGAACGGAGCCGGTTGGCGCCTACCGG
TGGATGTGGAATGTGTGCGAGGCCAGAGGCCACTTGTGTAGCGCCAAGTGCCCAGCG
GGGCTGCTAAAGCGCATGCTCCAGACTGCCTTGGGAAAAGCGCTCCCCTACCCATAA
CTTCGTATAATGTATGCTATACGAAGTTATTTTGCAGTTTTAAAATTATGTTTTAAAAT
GGACTATCATATGCTTACCGTAACTTGAAAGTATTTCGATTTCTTGGCTTTATATATCT
TGTGGAAAGGACGAAACACCGGGCACTCTTCCGTGATCTGGTGGATAAATTCGCAAG
GGTATCATGGCGGACGACCGGGATTCGAACCCCGGATCCGGCCGTCCGCCGTGATCC
ATGCGGTTACCGCCCGCGTGTCGAACCCAGGTGTGCGACGTCAGACAACGGGGGAGC
GCTCC
SEQ ID NO: 12 (Helper construct, v2)
GCCTCCACGGCCACTAGTCCATAGAGCCCACCGCATCCCCAGCATGCCTG
CTATTGTCTTCCCAATCCTCCCCCTTGCTGTCCTGCCCCACCCCACCCCCTAGAATAGA
ATGACACCTACTCAGACAATGCGATGCAATTTCCTCATTTTATTAGGAAAGGACAGTG
GGAGTGGCACCTTCCAGGGTCAAGGAAGGCACGGGGGAGGGGCAAACAACAGATGG
CTGGCAACTAGAAGGCACAGCTACATGGGGGTAGAGTCATAATCGTGCATCAGGATA
GGGCGGTGGTGCTGCAGCAGCGCGCGAATAAACTGCTGCCGCCGCCGCTCCGTCCTG
CAGGAATACAACATGGCAGTGGTCTCCTCAGCGATGATTCGCACCGCCCGCAGCATG
AGACGCCTTGTCCTCCGGGCACAGCAGCGCACCCTGATCTCACTTAAATCAGCACAG
TAACTGCAGCACAGCACCACAATATTGTTCAAAATCCCACAGTGCAAGGCGCTGTAT
CCAAAGCTCATGGCGGGGACCACAGAACCCACGTGGCCATCATACCACAAGCGCAG
GTAGATTAAGTGGCGACCCCTCATAAACACGCTGGACATAAACATTACCTCTTTTGGC
ATGTTGTAATTCACCACCTCCCGGTACCATATAAACCTCTGATTAAACATGGCGCCAT
CCACCACCATCCTAAACCAGCTGGCCAAAACCTGCCCGCCGGCTATGCACTGCAGGG
AACCGGGACTGGAACAATGACAGTGGAGAGCCCAGGACTCGTAACCATGGATCATC
ATGCTCGTCATGATATCAATGTTGGCACAACACAGGCACACGTGCATACACTTCCTCA
GGATTACAAGCTCCTCCCGCGTCAGAACCATATCCCAGGGAACAACCCATTCCTGAA
TCAGCGTAAATCCCACACTGCAGGGAAGACCTCGCACGTAACTCACGTTGTGCATTG
TCAAAGTGTTACATTCGGGCAGCAGCGGATGATCCTCCAGTATGGTAGCGCGGGTCT
CTGTCTCAAAAGGAGGTAGGCGATCCCTACTGTACGGAGTGCGCCGAGACAACCGAG
ATCGTGTTGGTCGTAGTGTCATGCCAAATGGAACGCCGGACGTAGTCATGGTTGTGG
CCATATTATCATCGTGTTTTTCAAAGGAAAACCACGTCCCCGTGGTTCGGGGGGCCTA
GACGTTTTTTTAACCTCGACTAAACACATGTAAAGCATGTGCACCGAGGCCCCAGATC
AGATCCCATACAATGGGGTACCTTCTGGGCATCCTTCAGCCCCTTGTTGAATACGCTT
GAGGAGAGCCATTTGACTCTTTCCACAACTATCCAACTCACAACGTGGCACTGGGGTT
GTGCCGCCTTTGCAGGTGTATCTTATACACGTGGCTTTTGGCCGCAGAGGCACCTGTC
GCCAGGTGGGGGGTTCCGCTGCCTGCAAAGGGTCGCTACAGACGTTGTTTGTCTTCAA
GAAGCTTCCAGAGGAACTGCTTCCTTCACGACATTCAACAGACCTTGCATTCCTTTGG
CGAGAGGGGAAAGACCCCTAGGAATGCTCGTCAAGAAGACAGGGCCAGGTTTCCGG
GCCCTCACATTGCCAAAAGACGGCAATATGGTGGAAAATAACATATAGACAAACGCA
CACCGGCCTTATTCCAAGCGGCTTCGGCCAGTAACGTTAGGGGGGGGGGAGGGAGAG
GGGCTTAAAAATCAAAGGGGTTCTGCCGCGCATCACTATGCGCCACTGGCAGGGACA
CGTTGCGATACTGGTGTTTAGTGCTCCACTTAAACTCAGGCACAACCATCCGCGGCAG
CTCGGTGAAGTTTTCACTCCACAGGCTGCGCACCATCACCAACGCGTTTAGCAGGTCG
GGCGCCGATATCTTGAAGTCGCAGTTGGGGCCTCCGCCCTGCGCGCGCGAGTTGCGA
TACACAGGGTTGCAGCACTGGAACACTATCAGCGCCGGGTGGTGCACGCTGGCCAGC
ACGCTCTTGTCGGAGATCAGATCCGCGTCCAGGTCCTCCGCGTTGCTCAGGGCGAAC
GGAGTCAACTTTGGTAGCTGCCTTCCCAAAAAGGGTGCATGCCCAGGCTTTGAGTTGC
ACTCGCACCGTAGTGGCATCAGAAGGTGACCGTGCCCGGTCTGGGCGTTAGGATACA
GCGCCTGCATGAAAGCCTTGATCTGCTTAAAAGCCACCTGAGCCTTTGCGCCTTCAGA
GAAGAACATGCCGCAAGACTTGCCGGAAAACTGATTGGCCGGACAGGCCGCGTCATG
CACGCAGCACCTTGCGTCGGTGTTGGAGATCTGCACCACATTTCGGCCCCACCGGTTC
TTCACGATCTTGGCCTTGCTAGACTGCTCCTTCAGCGCGCGCTGCCCGTTTTCGCTCGT
CACATCCATTTCAATCACGTGCTCCTTATTTATCATAATGCTCCCGTGTAGACACTTAA
GCTCGCCTTCGATCTCAGCGCAGCGGTGCAGCCACAACGCGCAGCCCGTGGGCTCGT
GGTGCTTGTAGGTTACCTCTGCAAACGACTGCAGGTACGCCTGCAGGAATCGCCCCA
TCATCGTCACAAAGGTCTTGTTGCTGGTGAAGGTCAGCTGCAACCCGCGGTGCTCCTC
GTTTAGCCAGGTCTTGCATACGGCCGCCAGAGCTTCCACTTGGTCAGGCAGTAGCTTG
AAGTTTGCCTTTAGATCGTTATCCACGTGGTACTTGTCCATCAACGCGCGCGCAGCCT
CCATGCCCTTCTCCCACGCAGACACGATCGGCAGGCTCAGCGGGTTTATCACCGTGCT
TTCACTTTCCGCTTCACTGGACTCTTCCTTTTCCTCTTGCGTCCGCATACCCCGCGCCA
CTGGGTCGTCTTCATTCAGCCGCCGCACCGTGCGCTTACCTCCCTTGCCGTGCTTGATT
AGCACCGGTGGGTTGCTGAAACCCACCATTTGTAGCGCCACATCTTCTCTTTCTTCCT
CGCTGTCCACGATCACCTCTGGGGATGGCGGGCGCTCGGGCTTGGGAGAGGGGCGCT
TCTTTTTCTTTTTGGACGCAATGGCCAAATCCGCCGTCGAGGTCGATGGCCGCGGGCT
GGGTGTGCGCGGCACCAGCGCATCTTGTGACGAGTCTTCTTCGTCCTCGGACTCGAGA
CGCCGCCTCAGCCGCTTTTTTGGGGGCGCGCGCTTGTCGTCATCGTCTTTGTAGTCGG
GAGGCGGCGGCGACGGCGACGGGGACGACACGTCCTCCATGGTTGGTGGACGTCGC
GCCGCACCGCGTCCGCGCTCGGGGGTGGTTTCGCGCTGCTCCTCTTCCCGACTGGCCA
TGGTGGCCGAGGATAACTTCGTATATGGTTTCTTATACGAAGTTATGATCCAGACATG
ATAAGATACATTGATGAGTTTGGACAAACCACAACTAGAATGCAGTGAAAAAAATGC
TTTATTTGTGAAATTTGTGATGCTATTGCTTTATTTGTAACCATTATAAGCTGCAATAA
ACAAGTTAACAACAACAATTGCATTCATTTTATGTTTCAGGTTCAGGGGGAGGTGTGG
GAGGTTTTTTAAAGCAAGTAAAACCTCTACAAATGTGGTATGGCTGATTATGATCCTC
TAGAGTCGCAGATCTGCTACGTATCAAGCTGTGGCAGGGAAACCCTCTGCCTCCCCC
GTGATGTAATACTTTTGCAAGGAATGCGATGAAGTAGAGCCCGCAGTGGCCAAGTGG
CTTTGGTCCGTCTCCTCCACGGATGCCCCTCCACGGCTAGTGGGCGCATGTAGGCGGT
GGGCGTCCGCCGCCTCCAGCAGCAGGTCATAGAGGGGCACCACGTTCTTGCACTTCA
TGCTGTACAGATGCTCCATGCCTTTGTTACTCATGTGTCGGATGTGGGAGAGGATGAG
GAGGAGCTGGGCCAGCCGCTGGTGCTGCTGCTGCAGGGTCAGGCCTGCCTTGGCCAT
CAGGTGGATCAAAGTGTCTGTGATCTTGTCCAGGACTCGGTGGATATGGTCCTTCTCT
TCCAGAGACTTCAGGGTGCTGGACAGAAATGTGTACACTCCAGAATTAAGCAAAATA
ATAGATTTGAGGCACACAAACTCCTCTCCCTGCAGATTCATCATGCGGAACCGAGAT
GATGTAGCCAGCAGCATGTCGAAGATCTCCACCATGCCCTCTACACATTTTCCCTGGT
TCCTGTCCAAGAGCAAGTTAGGAGCAAACAGTAGCTTCACTGGGTGCTCCATGGAGC
GCCAGACGAGACCAATCATCAGGATCTCTAGCCAGGCACATTCTAGAAGGTGGACCT
GATCATGGAGGGTCAAATCCACAAAGCCTGGCACCCTCTTCGCCCAGTTGATCATGT
GAACCAGCTCCCTGTCTGCCAGGTTGGTCAGTAAGCCCATCATCGAAGCTTCACTGAA
GGGTCTGGTAGGATCATACTCGGAATAGAGTATGGGGGGCTCAGCATCCAACAAGGC
ACTGACCATCTGGTCGGCCGTCAGGGACAAGGCCAGGCTGTTCTTCTTAGAGCGTTTG
ATCATGAGCGGGCTTGGCCAAAGGTTGGCAGCTCTCATGTCTCCAGCAGATGGCTCG
AGATCGCCATCTTCCAGCAGGCGCACCATTGCCCCTGTTTCACTATCCAGGTTACGGA
TATAGTTCATGACAATATTTACATTGGTCCAGCCACCAGCTTGCATGATCTCCGGTAT
TGAAACTCCAGCGCGGGCCATATCTCGCGCGGCTCCGACACGGGCACTGTGTCCAGA
CCAGGCCAGGTATCTCTGACCAGAGTCATCCTAAAATACACAAACAATTAGAATCAG
TAGTTTAACACATTATACACTTAAAAATTTTATATTTACCTTAGCGCCGTAAATCAAT
CGATGAGTTGCTTCAAAAATCCCTTCCAGGGCGCGAGTTGATAGCTGGCTGGTGGCA
GATGGCGCGGCAACACCATTTTTTCTGACCCGGCAAAACAGGTAGTTATTCGGATCAT
CAGCTACACCAGAGACGGAAATCCATCGCTCGACCAGTTTAGTGACTCCCAGGCTAA
GTGCCTTCTCTACACCTGCGGTGCTAACCAGCGTTTTCGTTCTGCCAATATGGATTAA
CATTCTCCCACCGTCAGTACGTGAGATATCTTTAACCCTGATCCTGGCAATTTCGGCT
ATACGTAACAGGGTGTTATAAGCAATCCCCAGAAATGCCAGATTACGTATATCCTGG
CAGCGATCGCTATTTTCCATGAGTGAACGGACTTGGTCGAAATCAGTGCGTTCGAAC
GCTAGAGCCTGTTTTGCACGTTCACCGGCATCAACGTTTTCTTTTCGGATCCGCCGCA
TAACCAGTGAAACAGCATTGCTGTCACTTGGTCGTGGCAGCCCGGACCGACGATGAA
GCATGTTTAGCTGGCCCAAATGTTGCTGGATAGTTTTTACTGCCAGACCGCGCGCTTG
AAGATATAGAAGATAATCGCGAACATCTTCAGGTTCTGCGGGAAACCATTTCCGGTT
ATTCAACTTGCACCATGCCGCCCACGACCGGCAAACGGACAGAAGCATTTTCCAGGT
ATGCTCAGAAAACGCCTGGCGATCCCTGAACATGTCCATCAGGTTCTTGCGAACCTCA
TCACTCGTTGCATCGACCGGTAATGCAGGCAAATTTTGGTGTACGGTCAGTAAATTGG
ACATGGTGGCTACGTAATAACTTCGTATATGGTTTCTTATACGAAGTTATGCGGCCGC
TTTACGAGGGTAGGAAGTGGTACGGAAAGTTGGTATAAGACAAAAGTGTTGTGGAAT
TGCTCCAGGCGATCTGACGGTTCACTAAACGAGCTCTGCTTTTATAGGCGCCCACCGT
ACACGCCTAAAGCTTATACGTTCTCTATCACTGATAGGGAGTAAACTGGATATACGTT
CTCTATCACTGATAGGGAGTAAACTGTAGATACGTTCTCTATCACTGATAGGGAGTAA
ACTGGTCATACGTTCTCTATCACTGATAGGGAGTAAACTCCTTATACGTTCTCTATCA
CTGATAGGGAGTAAAGTCTGCATACGTTCTCTATCACTGATAGGGAGTAAACTCTTCA
TACGTTCTCTATCACTGATAGGGAGTAAACTCGCGGCCGCAGAGAAATGTTCTGGCA
CCTGCACTTGCACTGGGGACAGCCTATTTTGCTAGTTTGTTTTGTTTCGTTTTGTTTTG
ATGGAGAGCGTATGTTAGTACTATCGATTCACACAAAAAACCAACACACAGATGTAA
TGAAAATAAAGATATTTTATTGGATCTGCGATCGCTCCGGTGCCCGTCAGTGGGCAG
AGCGCACATCGCCCACAGTCCCCGAGAAGTTGGGGGGAGGGGTCGGCAATTGAACG
GGTGCCTAGAGAAGGTGGCGCGGGGTAAACTGGGAAAGTGATGTCGTGTACTGGCTC
CGCCTTTTTCCCGAGGGTGGGGGAGAACCGTATGTAAGTGCAGTAGTCGCCGTGAAC
GTTCTTTTTCGCAACGGGTTTGCCGCCAGAACACAGCTGAAGCTTCGAGGGGCTCGCA
TCTCTCCTTCACGCGCCCGCCGCCCTACCTGAGGCCGCCATCCACGCCGGTTGAGTCG
CGTTCTGCCGCCTCCCGCCTGTGGTGCCTCCTGAACTGCGTCCGCCGTCTAGGTAAGT
TTAAAGCTCAGGTCGAGACCGGGCCTTTGTCCGGCGCTCCCTTGGAGCCTACCTAGAC
TCAGCCGGCTCTCCACGCTTTGCCTGACCCTGCTTGCTCAACTCTACGTCTTTGTTTCG
TTTTCTGTTCTGCGCCGTTACAGATCCAAGCTGTGACCGGCGCCTACGCTAGCGGATC
CGCCGCCACCATGTCTAGACTGGACAAGAGCAAAGTCATAAACTCTGCTCTGGAATT
ACTCAATGGAGTCGGTATCGAAGGCCTGACGACAAGGAAACTCGCTCAAAAGCTGGG
AGTTGAGCAGCCTACCCTGTACTGGCACGTGAAGAACAAGCGGGCCCTGCTCGATGC
CCTGCCAATCGAGATGCTGGACAGGCATCATACCCACTCCTGCCCCCTGGAAGGCGA
GTCATGGCAAGACTTTCTGCGGAACAACGCCAAGTCATACCGCTGTGCTCTTCTCTCA
CATCGCGACGGGGCTAAAGTGCATCTCGGCACCCGCCCAACAGAGAAACAGTACGA
AACCCTGGAAAATCAGCTCGCGTTCCTGTGTCAGCAAGGCTTCTCCCTGGAGAACGC
ACTGTACGCTCTGTCCGCCGTGGGCCACTTTACACTGGGCTGCGTATTGGAGGAACAG
GAGCATCAAGTAGCAAAAGAGGAAAGAGAGACACCTACCACCGATTCTATGCCCCC
ACTTCTGAAACAAGCAATTGAGCTGTTCGACCGGCAGGGAGCCGAACCTGCCTTCCT
TTTCGGCCTGGAACTAATCATATGTGGCCTGGAGAAACAGCTAAAGTGCGAAAGCGG
CGGGCCGACCGACGCCCTTGACGATTTTGACTTAGACATGCTCCCAGCCGATGCCCTT
GACGACTTTGACCTTGATATGCTGCCTGCTGACGCTCTTGACGATTTTGACCTTGACA
TGCTCCCCGGGGGATCCGGAGCTACTAACTTCAGCCTGCTGAAGCAGGCTGGAGACG
TGGAGGAGAACCCTGGACCTATGACAGAATATAAACCTACTGTCAGACTGGCAACTC
GAGACGACGTCCCTAGGGCCGTGAGAACATTGGCTGCCGCTTTCGCGGATTATCCCG
CTACACGCCACACAGTTGATCCTGATAGACATATTGAACGGGTTACAGAATTGCAAG
AACTTTTTTTGACCAGGGTAGGATTGGACATCGGTAAAGTTTGGGTCGCCGACGACG
GGGCTGCAGTGGCAGTGTGGACGACTCCGGAGAGCGTTGAGGCCGGGGCTGTATTTG
CAGAAATTGGTCCCCGAATGGCTGAGCTTAGTGGCTCTCGTCTCGCGGCTCAGCAAC
AAATGGAAGGACTCCTCGCCCCTCACCGCCCTAAAGAACCAGCTTGGTTCCTCGCTAC
TGTGGGCGTTAGCCCCGATCATCAGGGAAAGGGCCTTGGTTCCGCGGTGGTATTGCC
CGGAGTAGAAGCCGCAGAACGAGCCGGAGTGCCAGCCTTTCTTGAAACGTCAGCGCC
AAGGAATTTGCCCTTCTATGAACGGCTCGGATTTACAGTTACTGCTGACGTTGAAGTA
CCCGAGGGCCCACGGACGTGGTGCATGACGCGAAAACCCGGCGCTTGAGTTTAAACC
GCTGATCAGCCTCGACTGTGCCTTCTAGTTGCCAGCCATCTGTTGTTTGCCCCTCCCCC
GTGCCTTCCTTGACCCTGGAAGGTGCCACTCCCACTGTCCTTTCCTAATAAAATGAGG
AAATTGCATCGCATTGTCTGAGTAGGTGTCATTCTATTCTGGGGGGTGGGGGGGGCA
GGACAGCAAGGGGGAGGATTGGGAAGACAATAGCAGGCATGCTGGGGATGCGGTGG
GCTCTATGGCTTCTGAGGCGGAAAGAACCAGCTGGGGCTCGACTAGAGCTTGCGGAA
CCCTTAGTTTAAACGGGCCCTTAATTAATCGATGTAGGATGTTGCCCCTCCTGACGCG
GTAGGAGAAGGGGAGGGTGCCCTGCATGTCTGCCGCTGCTCTTGCTCTTGCCGCTGCT
GAGGAGGGGGGCGCATCTGCCGCAGCACCGGATGCATCTGGGAAAAGCAAAAAAGG
GGCTCGTCCCTGTTTCCGGAGGAATTTGCAAGCGGGGTCTTGCATGACGGGGAGGCA
AACCCCCGTTCGCCGCAGTCCGGCCGGCCCGAGACTCGAACCGGGGGTCCTGCGACT
CAACCCTTGGAAAATAACCCTCCGGCTACAGGGAGCGAGCCACTTAATGCTTTCGCTT
TCCAGCCTAACCGCTTACGCCGCGCGCGGCCAGTGGCCAAAAAAGCTAGCGCAGCAG
CCGCCGCGCCTGGAAGGAAGCCAAAAGGAGCGCTCCCCCGTTGTCTGACGTCGCACA
CCTGGGTTCGACACGCGGGCGGTAACCGCATGGATCACGGCGGACGGCCGGATCCGG
GGTTCGAACCCCGGTCGTCCGCCATGATACCCTTGCGAATTTATCCACCAGACCACGG
AAGAGTGCCCGCTTACAGGCTCTCCTTTTGCACGGTCTAGAGCGTCAACGACTGCGCA
CGCCTCACCGGCCAGAGCGTCCCGACCATGGAGCACTTTTTGCCGCTGCGCAACATCT
GGAACCGCGTCCGCGACTTTCCGCGCGCCTCCACCACCGCCGCCGGCATCACCTGGA
TGTCCAGGTACATCTACGGATTACGGGGCCCATTGGTATG
SEQ ID NO: 21 (reverse tetracycline-controlled transactivator mutant)
ATGTCTAGACTGGACAAGAGCAAAGTCATAAACTCTGCTCTGGAATTACT
CAATGGAGTCGGTATCGAAGGCCTGACGACAAGGAAACTCGCTCAAAAGCTGGGAG
TTGAGCAGCCTACCCTGTACTGGCACGTGAAGAACAAGCGGGCCCTGCTCGATGCCC
TGCCAATCGAGATGCTGGACAGGCATCATACCCACTCCTGCCCCCTGGAAGGCGAGT
CATGGCAAGACTTTCTGCGGAACAACGCCAAGTCATACCGCTGTGCTCTTCTCTCACA
TCGCGACGGGGCTAAAGTGCATCTCGGCACCCGCCCAACAGAGAAACAGTACGAAA
CCCTGGAAAATCAGCTCGCGTTCCTGTGTCAGCAAGGCTTCTCCCTGGAGAACGCACT
GTACGCTCTGTCCGCCGTGGGCCACTTTACACTGGGCTGCGTATTGGAGGAACAGGA
GCATCAAGTAGCAAAAGAGGAAAGAGAGACACCTACCACCGATTCTATGCCCCCACT
TCTGAAACAAGCAATTGAGCTGTTCGACCGGCAGGGAGCCGAACCTGCCTTCCTTTTC
GGCCTGGAACTAATCATATGTGGCCTGGAGAAACAGCTAAAGTGCGAAAGCGGCGG
GCCGACCGACGCCCTTGACGATTTTGACTTAGACATGCTCCCAGCCGATGCCCTTGAC
GACTTTGACCTTGATATGCTGCCTGCTGACGCTCTTGACGATTTTGACCTTGACATGCT
CCCCGGG
SEQ ID NO: 22 (Tet inducible promoter sequence)
GAGTTTACTCCCTATCAGTGATAGAGAACGTATGAAGAGTTTACTCCCTA
TCAGTGATAGAGAACGTATGCAGACTTTACTCCCTATCAGTGATAGAGAACGTATAA
GGAGTTTACTCCCTATCAGTGATAGAGAACGTATGACCAGTTTACTCCCTATCAGTGA
TAGAGAACGTATCTACAGTTTACTCCCTATCAGTGATAGAGAACGTATATCCAGTTTA
CTCCCTATCAGTGATAGAGAACGTATAAGCTTTAGGCGTGTACGGTGGGCGCCTATA
AAAGCAGAGCTCGTTTAGTGAACCGTCAGATCGCCTGGAGCAATTCCACAACACTTT
TGTCTTATACCAACTTTCCGTACCACTTCCTACCCTCGTAAA
SEQ ID NO: 23 (STXC0034)
GGTACCCAACTCCATGCTTAACAGTCCCCAGGTACAGCCCACCCTGCGTC
GCAACCAGGAACAGCTCTACAGCTTCCTGGAGCGCCACTCGCCCTACTTCCGCAGCC
ACAGTGCGCAGATTAGGAGCGCCACTTCTTTTTGTCACTTGAAAAACATGTAAAAAT
AATGTACTAGGAGACACTTTCAATAAAGGCAAATGTTTTTATTTGTACACTCTCGGGT
GATTATTTACCCCCCACCCTTGCCGTCTGCGCCGTTTAAAAATCAAAGGGGTTCTGCC
GCGCATCGCTATGCGCCACTGGCAGGGACACGTTGCGATACTGGTGTTTAGTGCTCCA
CTTAAACTCAGGCACAACCATCCGCGGCAGCTCGGTGAAGTTTTCACTCCACAGGCT
GCGCACCATCACCAACGCGTTTAGCAGGTCGGGCGCCGATATCTTGAAGTCGCAGTT
GGGGCCTCCGCCCTGCGCGCGCGAGTTGCGATACACAGGGTTGCAGCACTGGAACAC
TATCAGCGCCGGGTGGTGCACGCTGGCCAGCACGCTCTTGTCGGAGATCAGATCCGC
GTCCAGGTCCTCCGCGTTGCTCAGGGCGAACGGAGTCAACTTTGGTAGCTGCCTTCCC
AAAAAGGGTGCATGCCCAGGCTTTGAGTTGCACTCGCACCGTAGTGGCATCAGAAGG
TGACCGTGCCCGGTCTGGGCGTTAGGATACAGCGCCTGCATGAAAGCCTTGATCTGCT
TAAAAGCCACCTGAGCCTTTGCGCCTTCAGAGAAGAACATGCCGCAAGACTTGCCGG
AAAACTGATTGGCCGGACAGGCCGCGTCATGCACGCAGCACCTTGCGTCGGTGTTGG
AGATCTGCACCACATTTCGGCCCCACCGGTTCTTCACGATCTTGGCCTTGCTAGACTG
CTCCTTCAGCGCGCGCTGCCCGTTTTCGCTCGTCACATCCATTTCAATCACGTGCTCCT
TATTTATCATAATGCTCCCGTGTAGACACTTAAGCTCGCCTTCGATCTCAGCGCAGCG
GTGCAGCCACAACGCGCAGCCCGTGGGCTCGTGGTGCTTGTAGGTTACCTCTGCAAA
CGACTGCAGGTACGCCTGCAGGAATCGCCCCATCATCGTCACAAAGGTCTTGTTGCTG
GTGAAGGTCAGCTGCAACCCGCGGTGCTCCTCGTTTAGCCAGGTCTTGCATACGGCCG
CCAGAGCTTCCACTTGGTCAGGCAGTAGCTTGAAGTTTGCCTTTAGATCGTTATCCAC
GTGGTACTTGTCCATCAACGCGCGCGCAGCCTCCATGCCCTTCTCCCACGCAGACACG
ATCGGCAGGCTCAGCGGGTTTATCACCGTGCTTTCACTTTCCGCTTCACTGGACTCTTC
CTTTTCCTCTTGCGTCCGCATACCCCGCGCCACTGGGTCGTCTTCATTCAGCCGCCGCA
CCGTGCGCTTACCTCCCTTGCCGTGCTTGATTAGCACCGGTGGGTTGCTGAAACCCAC
CATTTGTAGCGCCACATCTTCTCTTTCTTCCTCGCTGTCCACGATCACCTCTGGGGATG
GCGGGCGCTCGGGCTTGGGAGAGGGGCGCTTCTTTTTCTTTTTGGACGCAATGGCCAA
ATCCGCCGTCGAGGTCGATGGCCGCGGGCTGGGTGTGCGCGGCACCAGCGCATCTTG
TGACGAGTCTTCTTCGTCCTCGGACTCGAGACGCCGCCTCAGCCGCTTTTTTGGGGGC
GCGCGGGGAGGCGGCGGCGACGGCGACGGGGACGACACGTCCTCCATGGTTGGTGG
ACGTCGCGCCGCACCGCGTCCGCGCTCGGGGGTGGTTTCGCGCTGCTCCTCTTCCCGA
CTGGCCATTTCCTTCTCCTATAGGCAGAAAAAGATCATGGAGTCAGTCGAGAAGGAG
GACAGCCTAACCGCCCCCTTTGAGTTCGCCACCACCGCCTCCACCGATGCCGCCAAC
GCGCCTACCACCTTCCCCGTCGAGGCACCCCCGCTTGAGGAGGAGGAAGTGATTATC
GAGCAGGACCCAGGTTTTGTAAGCGAAGACGACGAGGATCGCTCAGTACCAACAGA
GGATAAAAAGCAAGACCAGGACGACGCAGAGGCAAACGAGGAACAAGTCGGGCGG
GGGGACCAAAGGCATGGCGACTACCTAGATGTGGGAGACGACGTGCTGTTGAAGCAT
CTGCAGCGCCAGTGCGCCATTATCTGCGACGCGTTGCAAGAGCGCAGCGATGTGCCC
CTCGCCATAGCGGATGTCAGCCTTGCCTACGAACGCCACCTGTTCTCACCGCGCGTAC
CCCCCAAACGCCAAGAAAACGGCACATGCGAGCCCAACCCGCGCCTCAACTTCTACC
CCGTATTTGCCGTGCCAGAGGTGCTTGCCACCTATCACATCTTTTTCCAAAACTGCAA
GATACCCCTATCCTGCCGTGCCAACCGCAGCCGAGCGGACAAGCAGCTGGCCTTGCG
GCAGGGCGCTGTCATACCTGATATCGCCTCGCTCGACGAAGTGCCAAAAATCTTTGA
GGGTCTTGGACGCGACGAGAAACGCGCGGCAAACGCTCTGCAACAAGAAAACAGCG
AAAATGAAAGTCACTGTGGAGTGCTGGTGGAACTTGAGGGTGACAACGCGCGCCTAG
CCGTGCTGAAACGCAGCATCGAGGTCACCCACTTTGCCTACCCGGCACTTAACCTACC
CCCCAAGGTTATGAGCACAGTCATGAGCGAGCTGATCGTGCGCCGTGCACGACCCCT
GGAGAGGGATGCAAACTTGCAAGAACAAACCGAGGAGGGCCTACCCGCAGTTGGCG
ATGAGCAGCTGGCGCGCTGGCTTGAGACGCGCGAGCCTGCCGACTTGGAGGAGCGAC
GCAAGCTAATGATGGCCGCAGTGCTTGTTACCGTGGAGCTTGAGTGCATGCAGCGGT
TCTTTGCTGACCCGGAGATGCAGCGCAAGCTAGAGGAAACGTTGCACTACACCTTTC
GCCAGGGCTACGTGCGCCAGGCCTGCAAAATTTCCAACGTGGAGCTCTGCAACCTGG
TCTCCTACCTTGGAATTTTGCACGAAAACCGCCTCGGGCAAAACGTGCTTCATTCCAC
GCTCAAGGGCGAGGCGCGCCGCGACTACGTCCGCGACTGCGTTTACTTATTTCTGTGC
TACACCTGGCAAACGGCCATGGGCGTGTGGCAGCAATGCCTGGAGGAGCGCAACCTA
AAGGAGCTGCAGAAGCTGCTAAAGCAAAACTTGAAGGACCTATGGACGGCCTTCAAC
GAGCGCTCCGTGGCCGCGCACCTGGCGGACATTATCTTCCCCGAACGCCTGCTTAAA
ACCCTGCAACAGGGTCTGCCAGACTTCACCAGTCAAAGCATGTTGCAAAACTTTAGG
AACTTTATCCTAGAGCGTTCAGGAATTCTGCCCGCCACCTGCTGTGCGCTTCCTAGCG
ACTTTGTGCCCATTAAGTACCGTGAATGCCCTCCGCCGCTTTGGGGTCACTGCTACCT
TCTGCAGCTAGCCAACTACCTTGCCTACCACTCCGACATCATGGAAGACGTGAGCGG
TGACGGCCTACTGGAGTGTCACTGTCGCTGCAACCTATGCACCCCGCACCGCTCCCTG
GTCTGCAATTCGCAACTGCTTAGCGAAAGTCAAATTATCGGTACCTTTGAGCTGCAGG
GTCCCTCGCCTGACGAAAAGTCCGCGGCTCCGGGGTTGAAACTCACTCCGGGGCTGT
GGACGTCGGCTTACCTTCGCAAATTTGTACCTGAGGACTACCACGCCCACGAGATTA
GGTTCTACGAAGACCAATCCCGCCCGCCAAATGCGGAGCTTACCGCCTGCGTCATTA
CCCAGGGCCACATCCTTGGCCAATTGCAAGCCATCAACAAAGCCCGCCAAGAGTTTC
TGCTACGAAAGGGACGGGGGGTTTACCTGGACCCCCAGTCCGGCGAGGAGCTCAACC
CAATCCCCCCGCCGCCGCAGCCCTATCAGCAGCCGCGGGCCCTTGCTTCCCAGGATG
GCACCCAAAAAGAAGCTGCAGCTGCCGCCGCCGCCACCCACGGACGAGGAGGAATA
CTGGGACAGTCAGGCAGAGGAGGTTTTGGACGAGGAGGAGGAGATGATGGAAGACT
GGGACAGCCTAGACGAAGCTTCCGAGGCCGAAGAGGTGTCAGACGAAACACCGTCA
CCCTCGGTCGCATTCCCCTCGCCGGCGCCCCAGAAATTGGCAACCGTTCCCAGCATCG
CTACAACCTCCGCTCCTCAGGCGCCGCCGGCACTGCCTGTTCGCCGACCCAACCGTAG
ATGGGACACCACTGGAACCAGGGCCGGTAAGTCTAAGCAGCCGCCGCCGTTAGCCCA
AGAGCAACAACAGCGCCAAGGCTACCGCTCGTGGCGCGGGCACAAGAACGCCATAG
TTGCTTGCTTGCAAGACTGTGGGGGCAACATCTCCTTCGCCCGCCGCTTTCTTCTCTAC
CATCACGGCGTGGCCTTCCCCCGTAACATCCTGCATTACTACCGTCATCTCTACAGCC
CCTACTGCACCGGCGGCAGCGGCAGCGGCAGCAACAGCAGCGGTCACACAGAAGCA
AAGGCGACCGGATAGCAAGACTCTGACAAAGCCCAAGAAATCCACAGCGGCGGCAG
CAGCAGGAGGAGGAGCGCTGCGTCTGGCGCCCAACGAACCCGTATCGACCCGCGAG
CTTAGAAATAGGATTTTTCCCACTCTGTATGCTATATTTCAACAAAGCAGGGGCCAAG
AACAAGAGCTGAAAATAAAAAACAGGTCTCTGCGCTCCCTCACCCGCAGCTGCCTGT
ATCACAAAAGCGAAGATCAGCTTCGGCGCACGCTGGAAGACGCGGAGGCTCTCTTCA
GCAAATACTGCGCGCTGACTCTTAAGGACTAGTTTCGCGCCCTTTCTCAAATTTAAGC
GCGAAAACTACGTCATCTCCAGCGGCCACACCCGGCGCCAGCACCTGTCGTCAGCGC
CATTATGAGCAAGGAAATTCCCACGCCCTACATGTGGAGTTACCAGCCACAAATGGG
ACTTGCGGCTGGAGCTGCCCAAGACTACTCAACCCGAATAAACTACATGAGCGCGGG
ACCCCACATGATATCCCGGGTCAACGGAATCCGCGCCCACCGAAACCGAATTCTCCT
CGAACAGGCGGCTATTACCACCACACCTCGTAATAACCTTAATCCCCGTAGTTGGCCC
GCTGCCCTGGTGTACCAGGAAAGTCCCGCTCCCACCACTGTGGTACTTCCCAGAGAC
GCCCAGGCCGAAGTTCAGATGACTAACTCAGGGGCGCAGCTTGCGGGCGGCTTTCGT
CACAGGGTGCGGTCGCCCGGGCGTTTTAGGGCGGAGTAACTTGCATGTATTGGGAAT
TGTAGTTTTTTTAAAATGGGAAGTGACGTATCGTGGGAAAACGGAAGTGAAGATTTG
AGGAAGTTGTGGGTTTTTTGGCTTTCGTTTCTGGGCGTAGGTTCGCGTGCGGTTTTCTG
GGTGTTTTTTGTGGACTTTAACCGTTACGTCATTTTTTAGTCCTATATATACTCGCTCT
GTACTTGGCCCTTTTTACACTGTGACTGATTGAGCTGGTGCCGTGTCGAGTGGTGTTTT
TTAATAGGTTTTTTTACTGGTAAGGCTGACTGTTATGGCTGCCGCTGTGGAAGCGCTG
TATGTTGTTCTGGAGCGGGAGGGTGCTATTTTGCCTAGGCAGGAGGGTTTTTCAGGTG
TTTATGTGTTTTTCTCTCCTATTAATTTTGTTATACCTCCTATGGGGGCTGTAATGTTGT
CTCTACGCCTGCGGGTATGTATTCCCCCGGGCTATTTCGGTCGCTTTTTAGCACTGACC
GATGTTAACCAACCTGATGTGTTTACCGAGTCTTACATTATGACTCCGGACATGACCG
AGGAACTGTCGGTGGTGCTTTTTAATCACGGTGACCAGTTTTTTTACGGTCACGCCGG
CATGGCCGTAGTCCGTCTTATGCTTATAAGGGTTGTTTTTCCTGTTGTAAGACAGGCTT
CTAATGTTTAAATGTTTTTTTTTTTGTTATTTTATTTTGTGTTTAATGCAGGAACCCGCA
GACATGTTTGAGAGAAAAATGGTGTCTTTTTCTGTGGTGGTTCCGGAACTTACCTGCC
TTTATCTGCATGAGCATGACTACGATGTGCTTGCTTTTTTGCGCGAGGCTTTGCCTGAT
TTTTTGAGCAGCACCTTGCATTTTATATCGCCGCCCATGCAACAAGCTTACATAGGGG
CTACGCTGGTTAGCATAGCTCCGAGTATGCGTGTCATAATCAGTGTGGGTTCTTTTGT
CATGGTTCCTGGCGGGGAAGTGGCCGCGCTGGTCCGTGCAGACCTGCACGATTATGT
TCAGCTGGCCCTGCGAAGGGACCTACGGGATCGCGGTATTTTTGTTAATGTTCCGCTT
TTGAATCTTATACAGGTCTGTGAGGAACCTGAATTTTTGCAATCATGATTCGCTGCTT
GAGGCTGAAGGTGGAGGGCGCTCTGGAGCAGATTTTTACAATGGCCGGACTTAATAT
TCGGGATTTGCTTAGAGACATATTGATAAGGTGGCGAGATGAAAATTATTTGGGCAT
GGTTGAAGGTGCTGGAATGTTTATAGAGGAGATTCACCCTGAAGGGTTTAGCCTTTAC
GTCCACTTGGACGTGAGGGCAGTTTGCCTTTTGGAAGCCATTGTGCAACATCTTACAA
ATGCCATTATCTGTTCTTTGGCTGTAGAGTTTGACCACGCCACCGGAGGGGAGCGCGT
TCACTTAATAGATCTTCATTTTGAGGTTTTGGATAATCTTTTGGAATAAAAAAAAAAA
AACATGGTTCTTCCAGCTCTTCCCGCTCCTCCCGTGTGTGACTCGCAGAACGAATGTG
TAGGTTGGCTGGGTGTGGCTTATTCTGCGGTGGTGGATGTTATCAGGGCAGCGGCGC
ATGAAGGAGTTTACATAGAACCCGAAGCCAGGGGGCGCCTGGATGCTTTGAGAGAGT
GGATATACTACAACTACTACACAGAGCGAGCTAAGCGACGAGACCGGAGACGCAGA
TCTGTTTGTCACGCCCGCACCTGGTTTTGCTTCAGGAAATATGACTACGTCCGGCGTT
CCATTTGGCATGACACTACGACCAACACGATCTCGGTTGTCTCGGCGCACTCCGTACA
GTAGGGATCGCCTACCTCCTTTTGAGACAGAGACCCGCGCTACCATACTGGAGGATC
ATCCGCTGCTGCCCGAATGTAACACTTTGACAATGCACAACGTGAGTTACGTGCGAG
GTCTTCCCTGCAGTGTGGGATTTACGCTGATTCAGGAATGGGTTGTTCCCTGGGATAT
GGTTCTGACGCGGGAGGAGCTTGTAATCCTGAGGAAGTGTATGCACGTGTGCCTGTG
TTGTGCCAACATTGATATCATGACGAGCATGATGATCCATGGTTACGAGTCCTGGGCT
CTCCACTGTCATTGTTCCAGTCCCGGTTCCCTGCAGTGCATAGCCGGCGGGCAGGTTT
TGGCCAGCTGGTTTAGGATGGTGGTGGATGGCGCCATGTTTAATCAGAGGTTTATATG
GTACCGGGAGGTGGTGAATTACAACATGCCAAAAGAGGTAATGTTTATGTCCAGCGT
GTTTATGAGGGGTCGCCACTTAATCTACCTGCGCTTGTGGTATGATGGCCACGTGGGT
TCTGTGGTCCCCGCCATGAGCTTTGGATACAGCGCCTTGCACTGTGGGATTTTGAACA
ATATTGTGGTGCTGTGCTGCAGTTACTGTGCTGATTTAAGTGAGATCAGGGTGCGCTG
CTGTGCCCGGAGGACAAGGCGTCTCATGCTGCGGGCGGTGCGAATCATCGCTGAGGA
GACCACTGCCATGTTGTATTCCTGCAGGACGGAGCGGCGGCGGCAGCAGTTTATTCG
CGCGCTGCTGCAGCACCACCGCCCTATCCTGATGCACGATTATGACTCTACCCCCATG
TAGGCGTGGACTTCCCCTTCGCCGCCCGTTGAGCAACCGCAAGTTGGACAGCAGCCT
GTGGCTCAGCAGCTGGACAGCGACATGAACTTAAGCGAGCTGCCCGGGGAGTTTATT
AATATCACTGATGAGCGTTTGGCTCGACAGGAAACCGTGTGGAATATAACACCTAAG
AATATGTCTGTTACCCATGATATGATGCTTTTTAAGGCCAGCCGGGGAGAAAGGACT
GTGTACTCTGTGTGTTGGGAGGGAGGTGGCAGGTTGAATACTAGGGTTCTGTGAGTTT
GATTAAGGTACGGTGATCAATATAAGCTATGTGGTGGTGGGGCTATACTACTGAATG
AAAAATGACTTGAAATTTTCTGCAATTGAAAAATAAACACGTTGAAACATAACATGC
AACAGGTTCACGATTCTTTATTCCTGGGCAATGTAGGAGAAGGTGTAAGAGTTGGTA
GCAAAAGTTTCAGTGGTGTATTTTCCACTTTCCCAGGACCATGTAAAAGACATAGAGT
AAGTGCTTACCTCGCTAGTTTCTGTGGATTCACTAGAATCGATGTCGACGGGCACTCT
TCCGTGGTCTGGTGGATAAATTCGCAAGGGTATCATGGCGGACGAACCGGTACCGAA
CCCCGGATCCGGCCGTCCGCCGTGATCCATGCGGTTACCGCCCGCGTGTCGAACCCA
GGTGTGCGACGTCAGACAACGGGGGAGCGCTCCTTTTTCATATGATCAGCTCACTCA
AAGGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCGT
GCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGCTGCGCCTTA
TCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCTCT
GCTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAA
AGGATCTCAAGAAGATCCTTTGATCTTTTCTACGGGGTCTGACGCTCAGTGGAACGAA
AACTCACGTTAAGGGATTTTGGTCATGAGATTATCAAAAAGGATCTTCACCTAGATCC
TTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTT
ATCCGCCTCCATCCAGTCTATTAATTGTTGCCGGGAAGCTAGAGTAAGTAGTTCGCCA
GTTAATAGTTTGCGCAACGTTGTTGCCATTGCTACAGGCATCGTGGTGTCACGCTCGT
CGTTTGGTATGGCTTCATTCAGCTCCGGTTCCCAACGATCAAGGCGAGTTACATGATC
CCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTG
TCATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATAC
CGCGCCACATAGCAGAACTTTAAAAGTGCTCATCATTGGAAAACGTTCTTCGGGGCG
AAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAG
GAAGGCAAAATGCCGCAAAAAAGGGAATAAGGGCGACACGGAAATGTTGAATACTC
ATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCC
CGAAAAGTGCCACCTAAATTGTAAGCGTTAATATTTTGTTAAAATTCGCGTTAAATTT
TTGTTAAATCAGCTCATTTTTTAACCAATAGGCCGAAATCGGCAAAATCCCTTATAAA
TCAAAAGAATAGACCGAGATAGGGTTGAGTGTTGTTCCAGTTTGGAACAAGAGTCCA
CTATTAAAGAACGTGGACTCCAACGTCAAAGGGCGAAAAACCGTCTATCAGGGCGAT
GGCCCACTACGTGAACCATCACCCTAATCAAGTTTTTTGGGGTCGAGGTGCCGTAAA
GCACTAAATCGGAACCCTAAAGGGAGCCCCCGATTTAGAGCTTGACGGGGAAAGCCG
GCGAACGTGGCGAGAAAGGAAGGGAAGAAAGCGAAAGGAGCGGGCGCTAGGGCGC
TGGCAAGTGTAGCGGTCACGCTGCGCGTAACCACCACACCCGCCGCGCTTAATGCGC
CGCTACAGGGCGCGATGGATCC
SEQ ID NO: 24 (STXC0036)
GGTACCCAACTCCATGCTTAACAGTCCCCAGGTACAGCCCACCCTGCGTC
GCAACCAGGAACAGCTCTACAGCTTCCTGGAGCGCCACTCGCCCTACTTCCGCAGCC
ACAGTGCGCAGATTAGGAGCGCCACTTCTTTTTGTCACTTGAAAAACATGTAAAAAT
AATGTACTAGGAGACACTTTCAATAAAGGCAAATGTTTTTATTTGTACACTCTCGGGT
GATTATTTACCCCCCACCCTTGCCGTCTGCGCCGTTTAAAAATCAAAGGGGTTCTGCC
GCGCATCGCTATGCGCCACTGGCAGGGACACGTTGCGATACTGGTGTTTAGTGCTCCA
CTTAAACTCAGGCACAACCATCCGCGGCAGCTCGGTGAAGTTTTCACTCCACAGGCT
GCGCACCATCACCAACGCGTTTAGCAGGTCGGGCGCCGATATCTTGAAGTCGCAGTT
GGGGCCTCCGCCCTGCGCGCGCGAGTTGCGATACACAGGGTTGCAGCACTGGAACAC
TATCAGCGCCGGGTGGTGCACGCTGGCCAGCACGCTCTTGTCGGAGATCAGATCCGC
GTCCAGGTCCTCCGCGTTGCTCAGGGCGAACGGAGTCAACTTTGGTAGCTGCCTTCCC
AAAAAGGGTGCATGCCCAGGCTTTGAGTTGCACTCGCACCGTAGTGGCATCAGAAGG
TGACCGTGCCCGGTCTGGGCGTTAGGATACAGCGCCTGCATGAAAGCCTTGATCTGCT
TAAAAGCCACCTGAGCCTTTGCGCCTTCAGAGAAGAACATGCCGCAAGACTTGCCGG
AAAACTGATTGGCCGGACAGGCCGCGTCATGCACGCAGCACCTTGCGTCGGTGTTGG
AGATCTGCACCACATTTCGGCCCCACCGGTTCTTCACGATCTTGGCCTTGCTAGACTG
CTCCTTCAGCGCGCGCTGCCCGTTTTCGCTCGTCACATCCATTTCAATCACGTGCTCCT
TATTTATCATAATGCTCCCGTGTAGACACTTAAGCTCGCCTTCGATCTCAGCGCAGCG
GTGCAGCCACAACGCGCAGCCCGTGGGCTCGTGGTGCTTGTAGGTTACCTCTGCAAA
CGACTGCAGGTACGCCTGCAGGAATCGCCCCATCATCGTCACAAAGGTCTTGTTGCTG
GTGAAGGTCAGCTGCAACCCGCGGTGCTCCTCGTTTAGCCAGGTCTTGCATACGGCCG
CCAGAGCTTCCACTTGGTCAGGCAGTAGCTTGAAGTTTGCCTTTAGATCGTTATCCAC
GTGGTACTTGTCCATCAACGCGCGCGCAGCCTCCATGCCCTTCTCCCACGCAGACACG
ATCGGCAGGCTCAGCGGGTTTATCACCGTGCTTTCACTTTCCGCTTCACTGGACTCTTC
CTTTTCCTCTTGCGTCCGCATACCCCGCGCCACTGGGTCGTCTTCATTCAGCCGCCGCA
CCGTGCGCTTACCTCCCTTGCCGTGCTTGATTAGCACCGGTGGGTTGCTGAAACCCAC
CATTTGTAGCGCCACATCTTCTCTTTCTTCCTCGCTGTCCACGATCACCTCTGGGGATG
GCGGGCGCTCGGGCTTGGGAGAGGGGCGCTTCTTTTTCTTTTTGGACGCAATGGCCAA
ATCCGCCGTCGAGGTCGATGGCCGCGGGCTGGGTGTGCGCGGCACCAGCGCATCTTG
TGACGAGTCTTCTTCGTCCTCGGACTCGAGACGCCGCCTCAGCCGCTTTTTTGGGGGC
GCGCGGGGAGGCGGCGGCGACGGCGACGGGGACGACACGTCCTCCATGGTTGGTGG
ACGTCGCGCCGCACCGCGTCCGCGCTCGGGGGTGGTTTCGCGCTGCTCCTCTTCCCGA
CTGGCCATTTCCTTCTCCTATAGGCAGAAAAAGATCATGGAGTCAGTCGAGAAGGAG
GACAGCCTAACCGCCCCCTTTGAGTTCGCCACCACCGCCTCCACCGATGCCGCCAAC
GCGCCTACCACCTTCCCCGTCGAGGCACCCCCGCTTGAGGAGGAGGAAGTGATTATC
GAGCAGGACCCAGGTTTTGTAAGCGAAGACGACGAGGATCGCTCAGTACCAACAGA
GGATAAAAAGCAAGACCAGGACGACGCAGAGGCAAACGAGGAACAAGTCGGGCGG
GGGGACCAAAGGCATGGCGACTACCTAGATGTGGGAGACGACGTGCTGTTGAAGCAT
CTGCAGCGCCAGTGCGCCATTATCTGCGACGCGTTGCAAGAGCGCAGCGATGTGCCC
CTCGCCATAGCGGATGTCAGCCTTGCCTACGAACGCCACCTGTTCTCACCGCGCGTAC
CCCCCAAACGCCAAGAAAACGGCACATGCGAGCCCAACCCGCGCCTCAACTTCTACC
CCGTATTTGCCGTGCCAGAGGTGCTTGCCACCTATCACATCTTTTTCCAAAACTGCAA
GATACCCCTATCCTGCCGTGCCAACCGCAGCCGAGCGGACAAGCAGCTGGCCTTGCG
GCAGGGCGCTGTCATACCTGATATCGCCTCGCTCGACGAAGTGCCAAAAATCTTTGA
GGGTCTTGGACGCGACGAGAAACGCGCGGCAAACGCTCTGCAACAAGAAAACAGCG
AAAATGAAAGTCACTGTGGAGTGCTGGTGGAACTTGAGGGTGACAACGCGCGCCTAG
CCGTGCTGAAACGCAGCATCGAGGTCACCCACTTTGCCTACCCGGCACTTAACCTACC
CCCCAAGGTTATGAGCACAGTCATGAGCGAGCTGATCGTGCGCCGTGCACGACCCCT
GGAGAGGGATGCAAACTTGCAAGAACAAACCGAGGAGGGCCTACCCGCAGTTGGCG
ATGAGCAGCTGGCGCGCTGGCTTGAGACGCGCGAGCCTGCCGACTTGGAGGAGCGAC
GCAAGCTAATGATGGCCGCAGTGCTTGTTACCGTGGAGCTTGAGTGCATGCAGCGGT
TCTTTGCTGACCCGGAGATGCAGCGCAAGCTAGAGGAAACGTTGCACTACACCTTTC
GCCAGGGCTACGTGCGCCAGGCCTGCAAAATTTCCAACGTGGAGCTCTGCAACCTGG
TCTCCTACCTTGGAATTTTGCACGAAAACCGCCTCGGGCAAAACGTGCTTCATTCCAC
GCTCAAGGGCGAGGCGCGCCGCGACTACGTCCGCGACTGCGTTTACTTATTTCTGTGC
TACACCTGGCAAACGGCCATGGGCGTGTGGCAGCAATGCCTGGAGGAGCGCAACCTA
AAGGAGCTGCAGAAGCTGCTAAAGCAAAACTTGAAGGACCTATGGACGGCCTTCAAC
GAGCGCTCCGTGGCCGCGCACCTGGCGGACATTATCTTCCCCGAACGCCTGCTTAAA
ACCCTGCAACAGGGTCTGCCAGACTTCACCAGTCAAAGCATGTTGCAAAACTTTAGG
AACTTTATCCTAGAGCGTTCAGGAATTCTGCCCGCCACCTGCTGTGCGCTTCCTAGCG
ACTTTGTGCCCATTAAGTACCGTGAATGCCCTCCGCCGCTTTGGGGTCACTGCTACCT
TCTGCAGCTAGCCAACTACCTTGCCTACCACTCCGACATCATGGAAGACGTGAGCGG
TGACGGCCTACTGGAGTGTCACTGTCGCTGCAACCTATGCACCCCGCACCGCTCCCTG
GTCTGCAATTCGCAACTGCTTAGCGAAAGTCAAATTATCGGTACCTTTGAGCTGCAGG
GTCCCTCGCCTGACGAAAAGTCCGCGGCTCCGGGGTTGAAACTCACTCCGGGGCTGT
GGACGTCGGCTTACCTTCGCAAATTTGTACCTGAGGACTACCACGCCCACGAGATTA
GGTTCTACGAAGACCAATCCCGCCCGCCAAATGCGGAGCTTACCGCCTGCGTCATTA
CCCAGGGCCACATCCTTGGCCAATTGCAAGCCATCAACAAAGCCCGCCAAGAGTTTC
TGCTACGAAAGGGACGGGGGGTTTACCTGGACCCCCAGTCCGGCGAGGAGCTCAACC
CAATCCCCCCGCCGCCGCAGCCCTATCAGCAGCCGCGGGCCCTTGCTTCCCAGGATG
GCACCCAAAAAGAAGCTGCAGCTGCCGCCGCCGCCACCCACGGACGAGGAGGAATA
CTGGGACAGTCAGGCAGAGGAGGTTTTGGACGAGGAGGAGGAGATGATGGAAGACT
GGGACAGCCTAGACGAAGCTTCCGAGGCCGAAGAGGTGTCAGACGAAACACCGTCA
CCCTCGGTCGCATTCCCCTCGCCGGCGCCCCAGAAATTGGCAACCGTTCCCAGCATCG
CTACAACCTCCGCTCCTCAGGCGCCGCCGGCACTGCCTGTTCGCCGACCCAACCGTAG
ATGGGACACCACTGGAACCAGGGCCGGTAAGTCTAAGCAGCCGCCGCCGTTAGCCCA
AGAGCAACAACAGCGCCAAGGCTACCGCTCGTGGCGCGGGCACAAGAACGCCATAG
TTGCTTGCTTGCAAGACTGTGGGGGCAACATCTCCTTCGCCCGCCGCTTTCTTCTCTAC
CATCACGGCGTGGCCTTCCCCCGTAACATCCTGCATTACTACCGTCATCTCTACAGCC
CCTACTGCACCGGCGGCAGCGGCAGCGGCAGCAACAGCAGCGGTCACACAGAAGCA
AAGGCGACCGGATAGCAAGACTCTGACAAAGCCCAAGAAATCCACAGCGGCGGCAG
CAGCAGGAGGAGGAGCGCTGCGTCTGGCGCCCAACGAACCCGTATCGACCCGCGAG
CTTAGAAATAGGATTTTTCCCACTCTGTATGCTATATTTCAACAAAGCAGGGGCCAAG
AACAAGAGCTGAAAATAAAAAACAGGTCTCTGCGCTCCCTCACCCGCAGCTGCCTGT
ATCACAAAAGCGAAGATCAGCTTCGGCGCACGCTGGAAGACGCGGAGGCTCTCTTCA
GCAAATACTGCGCGCTGACTCTTAAGGACTAGTTTCGCGCCCTTTCTCAAATTTAAGC
GCGAAAACTACGTCATCTCCAGCGGCCACACCCGGCGCCAGCACCTGTCGTCAGCGC
CATTATGAGCAAGGAAATTCCCACGCCCTACATGTGGAGTTACCAGCCACAAATGGG
ACTTGCGGCTGGAGCTGCCCAAGACTACTCAACCCGAATAAACTACATGAGCGCGGG
ACCCCACATGATATCCCGGGTCAACGGAATCCGCGCCCACCGAAACCGAATTCTCCT
CGAACAGGCGGCTATTACCACCACACCTCGTAATAACCTTAATCCCCGTAGTTGGCCC
GCTGCCCTGGTGTACCAGGAAAGTCCCGCTCCCACCACTGTGGTACTTCCCAGAGAC
GCCCAGGCCGAAGTTCAGATGACTAACTCAGGGGCGCAGCTTGCGGGCGGCTTTCGT
CACAGGGTGCGGTCGCCCGGGCGTTTTAGGGCGGAGTAACTTGCATGTATTGGGAAT
TGTAGTTTTTTTAAAATGGGAAGTGACGTATCGTGGGAAAACGGAAGTGAAGATTTG
AGGAAGTTGTGGGTTTTTTGGCTTTCGTTTCTGGGCGTAGGTTCGCGTGCGGTTTTCTG
GGTGTTTTTTGTGGACTTTAACCGTTACGTCATTTTTTAGTCCTATATATACTCGCTCT
GTACTTGGCCCTTTTTACACTGTGACTGATTGAGCTGGTGCCGTGTCGAGTGGTGTTTT
TTAATAGGTTTTTTTACTGGTAAGGCTGACTGTTATGGCTGCCGCTGTGGAAGCGCTG
TATGTTGTTCTGGAGCGGGAGGGTGCTATTTTGCCTAGGCAGGAGGGTTTTTCAGGTG
TTTATGTGTTTTTCTCTCCTATTAATTTTGTTATACCTCCTATGGGGGCTGTAATGTTGT
CTCTACGCCTGCGGGTATGTATTCCCCCGGGCTATTTCGGTCGCTTTTTAGCACTGACC
GATGTTAACCAACCTGATGTGTTTACCGAGTCTTACATTATGACTCCGGACATGACCG
AGGAACTGTCGGTGGTGCTTTTTAATCACGGTGACCAGTTTTTTTACGGTCACGCCGG
CATGGCCGTAGTCCGTCTTATGCTTATAAGGGTTGTTTTTCCTGTTGTAAGACAGGCTT
CTAATGTTTAAATGTTTTTTTTTTTGTTATTTTATTTTGTGTTTAATGCAGGAACCCGCA
GACATGTTTGAGAGAAAAATGGTGTCTTTTTCTGTGGTGGTTCCGGAACTTACCTGCC
TTTATCTGCATGAGCATGACTACGATGTGCTTGCTTTTTTGCGCGAGGCTTTGCCTGAT
TTTTTGAGCAGCACCTTGCATTTTATATCGCCGCCCATGCAACAAGCTTACATAGGGG
CTACGCTGGTTAGCATAGCTCCGAGTATGCGTGTCATAATCAGTGTGGGTTCTTTTGT
CATGGTTCCTGGCGGGGAAGTGGCCGCGCTGGTCCGTGCAGACCTGCACGATTATGT
TCAGCTGGCCCTGCGAAGGGACCTACGGGATCGCGGTATTTTTGTTAATGTTCCGCTT
TTGAATCTTATACAGGTCTGTGAGGAACCTGAATTTTTGCAATCATGATTCGCTGCTT
GAGGCTGAAGGTGGAGGGCGCTCTGGAGCAGATTTTTACAATGGCCGGACTTAATAT
TCGGGATTTGCTTAGAGACATATTGATAAGGTGGCGAGATGAAAATTATTTGGGCAT
GGTTGAAGGTGCTGGAATGTTTATAGAGGAGATTCACCCTGAAGGGTTTAGCCTTTAC
GTCCACTTGGACGTGAGGGCAGTTTGCCTTTTGGAAGCCATTGTGCAACATCTTACAA
ATGCCATTATCTGTTCTTTGGCTGTAGAGTTTGACCACGCCACCGGAGGGGAGCGCGT
TCACTTAATAGATCTTCATTTTGAGGTTTTGGATAATCTTTTGGAATAAAAAAAAAAA
AACATGGTTCTTCCAGCTCTTCCCGCTCCTCCCGTGTGTGACTCGCAGAACGAATGTG
TAGGTTGGCTGGGTGTGGCTTATTCTGCGGTGGTGGATGTTATCAGGGCAGCGGCGC
ATGAAGGAGTTTACATAGAACCCGAAGCCAGGGGGCGCCTGGATGCTTTGAGAGAGT
GGATATACTACAACTACTACACAGAGCGAGCTAAGCGACGAGACCGGAGACGCAGA
TCTGTTTGTCACGCCCGCACCTGGTTTTGCTTCAGGAAATATGACTACGTCCGGCGTT
CCATTTGGCATGACACTACGACCAACACGATCTCGGTTGTCTCGGCGCACTCCGTACA
GTAGGGATCGCCTACCTCCTTTTGAGACAGAGACCCGCGCTACCATACTGGAGGATC
ATCCGCTGCTGCCCGAATGTAACACTTTGACAATGCACAACGTGAGTTACGTGCGAG
GTCTTCCCTGCAGTGTGGGATTTACGCTGATTCAGGAATGGGTTGTTCCCTGGGATAT
GGTTCTGACGCGGGAGGAGCTTGTAATCCTGAGGAAGTGTATGCACGTGTGCCTGTG
TTGTGCCAACATTGATATCATGACGAGCATGATGATCCATGGTTACGAGTCCTGGGCT
CTCCACTGTCATTGTTCCAGTCCCGGTTCCCTGCAGTGCATAGCCGGCGGGCAGGTTT
TGGCCAGCTGGTTTAGGATGGTGGTGGATGGCGCCATGTTTAATCAGAGGTTTATATG
GTACCGGGAGGTGGTGAATTACAACATGCCAAAAGAGGTAATGTTTATGTCCAGCGT
GTTTATGAGGGGTCGCCACTTAATCTACCTGCGCTTGTGGTATGATGGCCACGTGGGT
TCTGTGGTCCCCGCCATGAGCTTTGGATACAGCGCCTTGCACTGTGGGATTTTGAACA
ATATTGTGGTGCTGTGCTGCAGTTACTGTGCTGATTTAAGTGAGATCAGGGTGCGCTG
CTGTGCCCGGAGGACAAGGCGTCTCATGCTGCGGGCGGTGCGAATCATCGCTGAGGA
GACCACTGCCATGTTGTATTCCTGCAGGACGGAGCGGCGGCGGCAGCAGTTTATTCG
CGCGCTGCTGCAGCACCACCGCCCTATCCTGATGCACGATTATGACTCTACCCCCATG
TAGGCGTGGACTTCCCCTTCGCCGCCCGTTGAGCAACCGCAAGTTGGACAGCAGCCT
GTGGCTCAGCAGCTGGACAGCGACATGAACTTAAGCGAGCTGCCCGGGGAGTTTATT
AATATCACTGATGAGCGTTTGGCTCGACAGGAAACCGTGTGGAATATAACACCTAAG
AATATGTCTGTTACCCATGATATGATGCTTTTTAAGGCCAGCCGGGGAGAAAGGACT
GTGTACTCTGTGTGTTGGGAGGGAGGTGGCAGGTTGAATACTAGGGTTCTGTGAGTTT
GATTAAGGTACGGTGATCAATATAAGCTATGTGGTGGTGGGGCTATACTACTGAATG
AAAAATGACTTGAAATTTTCTGCAATTGAAAAATAAACACGTTGAAACATAACATGC
AACAGGTTCACGATTCTTTATTCCTGGGCAATGTAGGAGAAGGTGTAAGAGTTGGTA
GCAAAAGTTTCAGTGGTGTATTTTCCACTTTCCCAGGACCATGTAAAAGACATAGAGT
AAGTGCTTACCTCGCTAGTTTCTGTGGATTCACTAGAATCGATGTCGACGGGCACTCT
TCCGTGGTCTGGTGGATAAATTCGCAAGGGTATCATGGCGGACGACCGGGATTCGAA
CCCCGGATCCGGCCGTCCGCCGTGATCCATGCGGTTACCGCCCGCGTGTCGAACCCA
GGTGTGCGACGTCAGACAACGGGGGAGCGCTCCTTTTTCATATGATCAGCTCACTCA
AAGGCGGTAATACGGTTATCCACAGAATCAGGGGATAACGCAGGAAAGAACATGTG
AGCAAAAGGCCAGCAAAAGGCCAGGAACCGTAAAAAGGCCGCGTTGCTGGCGTTTTT
CCATAGGCTCCGCCCCCCTGACGAGCATCACAAAAATCGACGCTCAAGTCAGAGGTG
GCGAAACCCGACAGGACTATAAAGATACCAGGCGTTTCCCCCTGGAAGCTCCCTCGT
GCGCTCTCCTGTTCCGACCCTGCCGCTTACCGGATACCTGTCCGCCTTTCTCCCTTCGG
GAAGCGTGGCGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGCTGCGCCTTA
TCCGGTAACTATCGTCTTGAGTCCAACCCGGTAAGACACGACTTATCGCCACTGGCAG
CAGCCACTGGTAACAGGATTAGCAGAGCGAGGTATGTAGGCGGTGCTACAGAGTTCT
TGAAGTGGTGGCCTAACTACGGCTACACTAGAAGAACAGTATTTGGTATCTGCGCTCT
GCTGAAGCCAGTTACCTTCGGAAAAAGAGTTGGTAGCTCTTGATCCGGCAAACAAAC
CACCGCTGGTAGCGGTGGTTTTTTTGTTTGCAAGCAGCAGATTACGCGCAGAAAAAA
AGGATCTCAAGAAGATCCTTTGATCTTTTCTACGGGGTCTGACGCTCAGTGGAACGAA
AACTCACGTTAAGGGATTTTGGTCATGAGATTATCAAAAAGGATCTTCACCTAGATCC
TTTTAAATTAAAAATGAAGTTTTAAATCAATCTAAAGTATATATGAGTAAACTTGGTC
TGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTAC
CATCTGGCCCCAGTGCTGCAATGATACCGCGAGACCCACGCTCACCGGCTCCAGATTT
ATCAGCAATAAACCAGCCAGCCGGAAGGGCCGAGCGCAGAAGTGGTCCTGCAACTTT
ATCCGCCTCCATCCAGTCTATTAATTGTTGCCGGGAAGCTAGAGTAAGTAGTTCGCCA
GTTAATAGTTTGCGCAACGTTGTTGCCATTGCTACAGGCATCGTGGTGTCACGCTCGT
CGTTTGGTATGGCTTCATTCAGCTCCGGTTCCCAACGATCAAGGCGAGTTACATGATC
CCCCATGTTGTGCAAAAAAGCGGTTAGCTCCTTCGGTCCTCCGATCGTTGTCAGAAGT
AAGTTGGCCGCAGTGTTATCACTCATGGTTATGGCAGCACTGCATAATTCTCTTACTG
TCATGCCATCCGTAAGATGCTTTTCTGTGACTGGTGAGTACTCAACCAAGTCATTCTG
AGAATAGTGTATGCGGCGACCGAGTTGCTCTTGCCCGGCGTCAATACGGGATAATAC
CGCGCCACATAGCAGAACTTTAAAAGTGCTCATCATTGGAAAACGTTCTTCGGGGCG
AAAACTCTCAAGGATCTTACCGCTGTTGAGATCCAGTTCGATGTAACCCACTCGTGCA
CCCAACTGATCTTCAGCATCTTTTACTTTCACCAGCGTTTCTGGGTGAGCAAAAACAG
GAAGGCAAAATGCCGCAAAAAAGGGAATAAGGGCGACACGGAAATGTTGAATACTC
ATACTCTTCCTTTTTCAATATTATTGAAGCATTTATCAGGGTTATTGTCTCATGAGCGG
ATACATATTTGAATGTATTTAGAAAAATAAACAAATAGGGGTTCCGCGCACATTTCCC
CGAAAAGTGCCACCTAAATTGTAAGCGTTAATATTTTGTTAAAATTCGCGTTAAATTT
TTGTTAAATCAGCTCATTTTTTAACCAATAGGCCGAAATCGGCAAAATCCCTTATAAA
TCAAAAGAATAGACCGAGATAGGGTTGAGTGTTGTTCCAGTTTGGAACAAGAGTCCA
CTATTAAAGAACGTGGACTCCAACGTCAAAGGGCGAAAAACCGTCTATCAGGGCGAT
GGCCCACTACGTGAACCATCACCCTAATCAAGTTTTTTGGGGTCGAGGTGCCGTAAA
GCACTAAATCGGAACCCTAAAGGGAGCCCCCGATTTAGAGCTTGACGGGGAAAGCCG
GCGAACGTGGCGAGAAAGGAAGGGAAGAAAGCGAAAGGAGCGGGCGCTAGGGCGC
TGGCAAGTGTAGCGGTCACGCTGCGCGTAACCACCACACCCGCCGCGCTTAATGCGC
CGCTACAGGGCGCGATGGATCC
SEQ ID NO: 25 (STXC0030)
CCGCGGCCGCCAACTTTGTATAGAAAAGTTGTAGTTATTAATAGTAATCA
ATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTA
TTTACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGT
GATGCGGTTTTGGCAGTACATCAATGGGCGTGGATAGCGGTTTGACTCACGGGGATT
TCCAAGTCTCCACCCCATTGACGTCAATGGGAGTTTGTTTTGGCACCAAAATCAACGG
GACTTTCCAAAATGTCGTAACAACTCCGCCCCATTGACGCAAATGGGCGGTAGGCGT
GTACGGTGGGAGGTCTATATAAGCAGAGCTGGTTTAGTGAACCGTCAGATCCAAGTT
TGTACAAAAAAGCAGGCTGCCACCATGGCCAGTCGGGAAGAGGAGCAGCGCGAAAC
CACCCCCGAGCGCGGACGCGGTGCGGCGCGACGTCCACCAACCATGGAGGACGTGTC
GTCCCCGTCGCCGTCGCCGCCGCCTCCCCGCGCGCCCCCAAAAAAGCGGCTGAGGCG
GCGTCTCGAGTCCGAGGACGAAGAAGACTCGTCACAAGATGCGCTGGTGCCGCGCAC
ACCCAGCCCGCGGCCATCGACCTCGACGGCGGATTTGGCCATTGCGTCCAAAAAGAA
AAAGAAGCGCCCCTCTCCCAAGCCCGAGCGCCCGCCATCCCCAGAGGTGATCGTGGA
CAGCGAGGAAGAAAGAGAAGATGTGGCGCTACAAATGGTGGGTTTCAGCAACCCAC
CGGTGCTAATCAAGCACGGCAAGGGAGGTAAGCGCACGGTGCGGCGGCTGAATGAA
GACGACCCAGTGGCGCGGGGTATGCGGACGCAAGAGGAAAAGGAAGAGTCCAGTGA
AGCGGAAAGTGAAAGCACGGTGATAAACCCGCTGAGCCTGCCGATCGTGTCTGCGTG
GGAGAAGGGCATGGAGGCTGCGCGCGCGTTGATGGACAAGTACCACGTGGATAACG
ATCTAAAGGCAAACTTCAAGCTACTGCCTGACCAAGTGGAAGCTCTGGCGGCCGTAT
GCAAGACCTGGCTAAACGAGGAGCACCGCGGGTTGCAGCTGACCTTCACCAGCAACA
AGACCTTTGTGACGATGATGGGGCGATTCCTGCAGGCGTACCTGCAGTCGTTTGCAG
AGGTAACCTACAAGCACCACGAGCCCACGGGCTGCGCGTTGTGGCTGCACCGCTGCG
CTGAGATCGAAGGCGAGCTTAAGTGTCTACACGGGAGCATTATGATAAATAAGGAGC
ACGTGATTGAAATGGATGTGACGAGCGAAAACGGGCAGCGCGCGCTGAAGGAGCAG
TCTAGCAAGGCCAAGATCGTGAAGAACCGGTGGGGCCGAAATGTGGTGCAGATCTCC
AACACCGACGCAAGGTGCTGCGTGCATGACGCGGCCTGTCCGGCCAATCAGTTTTCC
GGCAAGTCTTGCGGCATGTTCTTCTCTGAAGGCGCAAAGGCTCAGGTGGCTTTTAAGC
AGATCAAGGCTTTCATGCAGGCGCTGTATCCTAACGCCCAGACCGGGCACGGTCACC
TTCTGATGCCACTACGGTGCGAGTGCAACTCAAAGCCTGGGCATGCACCCTTTTTGGG
AAGGCAGCTACCAAAGTTGACTCCGTTCGCCCTGAGCAACGCGGAGGACCTGGACGC
GGATCTGATCTCCGACAAGAGCGTGCTGGCCAGCGTGCACCACCCGGCGCTGATAGT
GTTCCAGTGCTGCAACCCTGTGTATCGCAACTCGCGCGCGCAGGGCGGAGGCCCCAA
CTGCGACTTCAAGATATCGGCGCCCGACCTGCTAAACGCGTTGGTGATGGTGCGCAG
CCTGTGGAGTGAAAACTTCACCGAGCTGCCGCGGATGGTTGTGCCTGAGTTTAAGTG
GAGCACTAAACACCAGTATCGCAACGTGTCCCTGCCAGTGGCGCATAGCGATGCGCG
GCAGAACCCCTTTGATTTTTAAACCCAGCTTTCTTGTACAAAGTGGGCCCCTCTCCCT
CCCCCCCCCCTAACGTTACTGGCCGAAGCCGCTTGGAATAAGGCCGGTGTGCGTTTGT
CTATATGTTATTTTCCACCATATTGCCGTCTTTTGGCAATGTGAGGGCCCGGAAACCT
GGCCCTGTCTTCTTGACGAGCATTCCTAGGGGTCTTTCCCCTCTCGCCAAAGGAATGC
AAGGTCTGTTGAATGTCGTGAAGGAAGCAGTTCCTCTGGAAGCTTCTTGAAGACAAA
CAACGTCTGTAGCGACCCTTTGCAGGCAGCGGAACCCCCCACCTGGCGACAGGTGCC
TCTGCGGCCAAAAGCCACGTGTATAAGATACACCTGCAAAGGCGGCACAACCCCAGT
GCCACGTTGTGAGTTGGATAGTTGTGGAAAGAGTCAAATGGCTCTCCTCAAGCGTATT
CAACAAGGGGCTGAAGGATGCCCAGAAGGTACCCCATTGTATGGGATCTGATCTGGG
GCCTCGGTGCACATGCTTTACATGTGTTTAGTCGAGGTTAAAAAAACGTCTAGGCCCC
CCGAACCACGGGGACGTGGTTTTCCTTTGAAAAACACGATGATAATATGGCCACAAC
CATGACTACGTCCGGCGTTCCATTTGGCATGACACTACGACCAACACGATCTCGGTTG
TCTCGGCGCACTCCGTACAGTAGGGATCGCCTACCTCCTTTTGAGACAGAGACCCGCG
CTACCATACTGGAGGATCATCCGCTGCTGCCCGAATGTAACACTTTGACAATGCACA
ACGTGAGTTACGTGCGAGGTCTTCCCTGCAGTGTGGGATTTACGCTGATTCAGGAATG
GGTTGTTCCCTGGGATATGGTTCTGACGCGGGAGGAGCTTGTAATCCTGAGGAAGTG
TATGCACGTGTGCCTGTGTTGTGCCAACATTGATATCATGACGAGCATGATGATCCAT
GGTTACGAGTCCTGGGCTCTCCACTGTCATTGTTCCAGTCCCGGTTCCCTGCAGTGCA
TAGCCGGCGGGCAGGTTTTGGCCAGCTGGTTTAGGATGGTGGTGGATGGCGCCATGT
TTAATCAGAGGTTTATATGGTACCGGGAGGTGGTGAATTACAACATGCCAAAAGAGG
TAATGTTTATGTCCAGCGTGTTTATGAGGGGTCGCCACTTAATCTACCTGCGCTTGTG
GTATGATGGCCACGTGGGTTCTGTGGTCCCCGCCATGAGCTTTGGATACAGCGCCTTG
CACTGTGGGATTTTGAACAATATTGTGGTGCTGTGCTGCAGTTACTGTGCTGATTTAA
GTGAGATCAGGGTGCGCTGCTGTGCCCGGAGGACAAGGCGTCTCATGCTGCGGGCGG
TGCGAATCATCGCTGAGGAGACCACTGCCATGTTGTATTCCTGCAGGACGGAGCGGC
GGCGGCAGCAGTTTATTCGCGCGCTGCTGCAGCACCACCGCCCTATCCTGATGCACG
ATTATGACTCTACCCCCATGTAGCAACTTTATTATACATAGTTGATGGCCGGCCGCTT
CGAGCAGACATGATAAGATACATTGATGAGTTTGGACAAACCACAACTAGAATGCAG
TGAAAAAAATGCTTTATTTGTGAAATTTGTGATGCTATTGCTTTATTTGTAACCATTAT
AAGCTGCAATAAACAAGTTAACAACAACAATTGCATTCATTTTATGTTTCAGGTTCAG
GGGGAGGTGTGGGAGGTTTTTTAAAGCAAGTAAAACCTCTACAAATGTGGTAGCGGC
CGCGGCGCTCTTCCGCTTCCTCGCTCACTGACTCGCTGCGCTCGGTCGTTCGGCTGCG
GCGAGCGGTATCAGCTCACTCAAAGGCGGTAATACGGTTATCCACAGAATCAGGGGA
TAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATAC
CTGTCCGCCTTTCTCTCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGG
GTCTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTA
TCTCAGCGATCTGTCTATTTCGTTCATCCATAGTTGCCTGACTCCCCGTCGTGTAGATA
ACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCCAA
CGATCAAGGCGAGTTACATGATCCCCCATGTTGTGCAAAAAAGCGGTTAGCTCCTTC
GGTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCC
CGGCGTCAATACGGGATAATACCGCGCCACATAGCAGAACTTTAAAAGTGCTCATCA
TTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAAGGG
CGACACGGAAATGTTGAATACTCATACTCTTCCTTTTTCAATATTATTGAAGCATTTAT
CAGGGTTATTGTCTCATGAGCGGATACATATTTGAATGTATTTAGAAAAATAAACAA
ATAGGGGTTCCGCGCACATTTCCCCGAAAAGTGCCACCTGACGTCTAAGAAACCATT
ATTATCATGACATTAACCTATAAAAATAGGCGTATCACGAGGCCCTTTCGTCGGCGCG
CCTCGATGTAGGATGTTGCCCCTCCTGACGCGGTAGGAGAAGGGGAGGGTGCCCTGC
ATGTCTGCCGCTGCTCTTGCTCTTGCCGCTGCTGAGGAGGGGGGCGCATCTGCCGCAG
CACCGGATGCATCTGGGAAAAGCAAAAAAGGGGCTCGTCCCTGTTTCCGGAGGAATT
TGCAAGCGGGGTCTTGCATGACGGGGAGGCAAACCCCCGTTCGCCGCAGTCCGGCCG
GCCCGAGACTCGAACCGGGGGTCCTGCGACTCAACCCTTGGAAAATAACCCTCCGGC
TACAGGGAGCGAGCCACTTAATGCTTTCGCTTTCCAGCCTAACCGCTTACGCCGCGCG
CGGCCAGTGGCCAAAAAAGCTAGCGCAGCAGCCGCCGCGCCTGGAAGGAAGCCAAA
AGGAGCGCTCCCCCGTTGTCTGACGTCGCACACCTGGGTTCGACACGCGGGCGGTAA
CCGCATGGATCACGGCGGACGGCCGGATCCGGGGTTCGAACCCCGGTCGTCCGCCAT
GATACCCTTGCGAATTTATCCACCAGACCACGGAAGAGTGCCCGCTTACAGGCTCTCC
TTTTGCACGGTCTAGAGCGTCAACGACTGCGCACGCCTCACCGGCCAGAGCGTCCCG
ACCATGGAGCACTTTTTGCCGCTGCGCAACATCTGGAACCGCGTCCGCGACTTTCCGC
GCGCCTCCACCACCGCCGCCGGCATCACCTGGATGTCCAGGTACATCTACGGATTAC
GGGCGCG
SEQ ID NO: 26 (STXC0031)
CCGCGGCCGCCAACTTTGTATAGAAAAGTTGTAGTTATTAATAGTAATCA
ATTACGGGGTCATTAGTTCATAGCCCATATATGGAGTTCCGCGTTACATAACTTACGG
TAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTA
TTTACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGACCT
TATGGGACTTTCCTACTTGGCAGTACATCTACGTATTAGTCATCGCTATTACCATGGT
GATGCGGTTTTGGCAGTACATCAATGGGCGTGGATAGCGGTTTGACTCACGGGGATT
TCCAAGTCTCCACCCCATTGACGTCAATGGGAGTTTGTTTTGGCACCAAAATCAACGG
GACTTTCCAAAATGTCGTAACAACTCCGCCCCATTGACGCAAATGGGCGGTAGGCGT
GTACGGTGGGAGGTCTATATAAGCAGAGCTGGTTTAGTGAACCGTCAGATCCAAGTT
TGTACAAAAAAGCAGGCTGCCACCATGACTACGTCCGGCGTTCCATTTGGCATGACA
CTACGACCAACACGATCTCGGTTGTCTCGGCGCACTCCGTACAGTAGGGATCGCCTAC
CTCCTTTTGAGACAGAGACCCGCGCTACCATACTGGAGGATCATCCGCTGCTGCCCGA
ATGTAACACTTTGACAATGCACAACGTGAGTTACGTGCGAGGTCTTCCCTGCAGTGTG
GGATTTACGCTGATTCAGGAATGGGTTGTTCCCTGGGATATGGTTCTGACGCGGGAG
GAGCTTGTAATCCTGAGGAAGTGTATGCACGTGTGCCTGTGTTGTGCCAACATTGATA
TCATGACGAGCATGATGATCCATGGTTACGAGTCCTGGGCTCTCCACTGTCATTGTTC
CAGTCCCGGTTCCCTGCAGTGCATAGCCGGCGGGCAGGTTTTGGCCAGCTGGTTTAGG
ATGGTGGTGGATGGCGCCATGTTTAATCAGAGGTTTATATGGTACCGGGAGGTGGTG
AATTACAACATGCCAAAAGAGGTAATGTTTATGTCCAGCGTGTTTATGAGGGGTCGC
CACTTAATCTACCTGCGCTTGTGGTATGATGGCCACGTGGGTTCTGTGGTCCCCGCCA
TGAGCTTTGGATACAGCGCCTTGCACTGTGGGATTTTGAACAATATTGTGGTGCTGTG
CTGCAGTTACTGTGCTGATTTAAGTGAGATCAGGGTGCGCTGCTGTGCCCGGAGGAC
AAGGCGTCTCATGCTGCGGGCGGTGCGAATCATCGCTGAGGAGACCACTGCCATGTT
GTATTCCTGCAGGACGGAGCGGCGGCGGCAGCAGTTTATTCGCGCGCTGCTGCAGCA
CCACCGCCCTATCCTGATGCACGATTATGACTCTACCCCCATGTAGACCCAGCTTTCT
TGTACAAAGTGGGCCCCTCTCCCTCCCCCCCCCCTAACGTTACTGGCCGAAGCCGCTT
GGAATAAGGCCGGTGTGCGTTTGTCTATATGTTATTTTCCACCATATTGCCGTCTTTTG
GCAATGTGAGGGCCCGGAAACCTGGCCCTGTCTTCTTGACGAGCATTCCTAGGGGTCT
TTCCCCTCTCGCCAAAGGAATGCAAGGTCTGTTGAATGTCGTGAAGGAAGCAGTTCCT
CTGGAAGCTTCTTGAAGACAAACAACGTCTGTAGCGACCCTTTGCAGGCAGCGGAAC
CCCCCACCTGGCGACAGGTGCCTCTGCGGCCAAAAGCCACGTGTATAAGATACACCT
GCAAAGGCGGCACAACCCCAGTGCCACGTTGTGAGTTGGATAGTTGTGGAAAGAGTC
AAATGGCTCTCCTCAAGCGTATTCAACAAGGGGCTGAAGGATGCCCAGAAGGTACCC
CATTGTATGGGATCTGATCTGGGGCCTCGGTGCACATGCTTTACATGTGTTTAGTCGA
GGTTAAAAAAACGTCTAGGCCCCCCGAACCACGGGGACGTGGTTTTCCTTTGAAAAA
CACGATGATAATATGGCCACAACCATGGCCAGTCGGGAAGAGGAGCAGCGCGAAAC
CACCCCCGAGCGCGGACGCGGTGCGGCGCGACGTCCACCAACCATGGAGGACGTGTC
GTCCCCGTCGCCGTCGCCGCCGCCTCCCCGCGCGCCCCCAAAAAAGCGGCTGAGGCG
GCGTCTCGAGTCCGAGGACGAAGAAGACTCGTCACAAGATGCGCTGGTGCCGCGCAC
ACCCAGCCCGCGGCCATCGACCTCGACGGCGGATTTGGCCATTGCGTCCAAAAAGAA
AAAGAAGCGCCCCTCTCCCAAGCCCGAGCGCCCGCCATCCCCAGAGGTGATCGTGGA
CAGCGAGGAAGAAAGAGAAGATGTGGCGCTACAAATGGTGGGTTTCAGCAACCCAC
CGGTGCTAATCAAGCACGGCAAGGGAGGTAAGCGCACGGTGCGGCGGCTGAATGAA
GACGACCCAGTGGCGCGGGGTATGCGGACGCAAGAGGAAAAGGAAGAGTCCAGTGA
AGCGGAAAGTGAAAGCACGGTGATAAACCCGCTGAGCCTGCCGATCGTGTCTGCGTG
GGAGAAGGGCATGGAGGCTGCGCGCGCGTTGATGGACAAGTACCACGTGGATAACG
ATCTAAAGGCAAACTTCAAGCTACTGCCTGACCAAGTGGAAGCTCTGGCGGCCGTAT
GCAAGACCTGGCTAAACGAGGAGCACCGCGGGTTGCAGCTGACCTTCACCAGCAACA
AGACCTTTGTGACGATGATGGGGCGATTCCTGCAGGCGTACCTGCAGTCGTTTGCAG
AGGTAACCTACAAGCACCACGAGCCCACGGGCTGCGCGTTGTGGCTGCACCGCTGCG
CTGAGATCGAAGGCGAGCTTAAGTGTCTACACGGGAGCATTATGATAAATAAGGAGC
ACGTGATTGAAATGGATGTGACGAGCGAAAACGGGCAGCGCGCGCTGAAGGAGCAG
TCTAGCAAGGCCAAGATCGTGAAGAACCGGTGGGGCCGAAATGTGGTGCAGATCTCC
AACACCGACGCAAGGTGCTGCGTGCATGACGCGGCCTGTCCGGCCAATCAGTTTTCC
GGCAAGTCTTGCGGCATGTTCTTCTCTGAAGGCGCAAAGGCTCAGGTGGCTTTTAAGC
AGATCAAGGCTTTCATGCAGGCGCTGTATCCTAACGCCCAGACCGGGCACGGTCACC
TTCTGATGCCACTACGGTGCGAGTGCAACTCAAAGCCTGGGCATGCACCCTTTTTGGG
AAGGCAGCTACCAAAGTTGACTCCGTTCGCCCTGAGCAACGCGGAGGACCTGGACGC
GGATCTGATCTCCGACAAGAGCGTGCTGGCCAGCGTGCACCACCCGGCGCTGATAGT
GTTCCAGTGCTGCAACCCTGTGTATCGCAACTCGCGCGCGCAGGGCGGAGGCCCCAA
CTGCGACTTCAAGATATCGGCGCCCGACCTGCTAAACGCGTTGGTGATGGTGCGCAG
CCTGTGGAGTGAAAACTTCACCGAGCTGCCGCGGATGGTTGTGCCTGAGTTTAAGTG
GAGCACTAAACACCAGTATCGCAACGTGTCCCTGCCAGTGGCGCATAGCGATGCGCG
GCAGAACCCCTTTGATTTTTAACAACTTTATTATACATAGTTGATGGCCGGCCGCTTC
GAGCAGACATGATAAGATACATTGATGAGTTTGGACAAACCACAACTAGAATGCAGT
GAAAAAAATGCTTTATTTGTGAAATTTGTGATGCTATTGCTTTATTTGTAACCATTATA
AGCTGCAATAAACAAGTTAACAACAACAATTGCATTCATTTTATGTTTCAGGTTCAGG
GGGAGGTGTGGGAGGTTTTTTAAAGCAAGTAAAACCTCTACAAATGTGGTAGCGGCC
GCGGCGCTCTTCCGCTTCCTCGCTCACTGACTCGCTGCGCTCGGTCGTTCGGCTGCGG
CGAGCGGTATCAGCTCACTCAAAGGCGGTAATACGGTTATCCACAGAATCAGGGGAT
AACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGTT
TCCCCCTGGAAGCTCCCTCGTGCGCTCTCCTGTTCCGACCCTGCCGCTTACCGGATAC
CTGTCCGCCTTTCTCTCTTCGGGAAGCGTGGCGCTTTCTCATAGCTCACGCTGTAGGT
ATCTCAGTTCGGTGTAGGTCGTTCGCTCCAAGCTGGGCTGTGTGCACGAACCCCCCGT
TCAGCCCGACCGCTGCGCCTTATCCGGTAACTATCGTCTTGAGTCCAACCCGGTAAGA
CACGACTTATCGCCACTGGCAGCAGCCACTGGTAACAGGATTAGCAGAGCGAGGTAT
GTAGGCGGTGCTACAGAGTTCTTGAAGTGGTGGCCTAACTACGGCTACACTAGAAGA
ACAGTATTTGGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGTTTGCAAGCA
GCAGATTACGCGCAGAAAAAAAGGATCTCAAGAAGATCCTTTGATCTTTTCTACGGG
GTCTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAATCTAA
AGTATATATGAGTAAACTTGGTCTGACAGTTACCAATGCTTAATCAGTGAGGCACCTA
TCTCAGCGATCTGTCTATTTCGTTCATCCATAGTTGCCTGACTCCCCGTCGTGTAGATA
ACTACGATACGGGAGGGCTTACCATCTGGCCCCAGTGCTGCAATGATACCGCGAGAC
CCACGCTCACCGGCTCCAGATTTATCAGCAATAAACCAGCCAGCCGGAAGGGCCGAG
CGCAGAAGTGGTCCTGCAACTTTATCCGCCTCCATCCAGTCTATTAATTGTTGCCGGG
AAGCTAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTCCCAA
CGATCAAGGCGAGTTACATGATCCCCCATGTTGTGCAAAAAAGCGGTTAGCTCCTTC
GGTCCTCCGATCGTTGTCAGAAGTAAGTTGGCCGCAGTGTTATCACTCATGGTTATGG
CAGCACTGCATAATTCTCTTACTGTCATGCCATCCGTAAGATGCTTTTCTGTGACTGGT
GAGTACTCAACCAAGTCATTCTGAGAATAGTGTATGCGGCGACCGAGTTGCTCTTGCC
CGGCGTCAATACGGGATAATACCGCGCCACATAGCAGAACTTTAAAAGTGCTCATCA
TTGGAAAACGTTCTTCGGGGCGAAAACTCTCAAGGATCTTACCGCTGTTGAGATCCA
GTTCGATGTAACCCACTCGTGCACCCAACTGATCTTCAGCATCTTTTACTTTCACCAG
CGTTTCTGGGTGAGCAAAAACAGGAAGGCAAAATGCCGCAAAAAAGGGAATAAGGG
CGACACGGAAATGTTGAATACTCATACTCTTCCTTTTTCAATATTATTGAAGCATTTAT
CAGGGTTATTGTCTCATGAGCGGATACATATTTGAATGTATTTAGAAAAATAAACAA
ATAGGGGTTCCGCGCACATTTCCCCGAAAAGTGCCACCTGACGTCTAAGAAACCATT
ATTATCATGACATTAACCTATAAAAATAGGCGTATCACGAGGCCCTTTCGTCGGCGCG
CCTCGATGTAGGATGTTGCCCCTCCTGACGCGGTAGGAGAAGGGGAGGGTGCCCTGC
ATGTCTGCCGCTGCTCTTGCTCTTGCCGCTGCTGAGGAGGGGGGCGCATCTGCCGCAG
CACCGGATGCATCTGGGAAAAGCAAAAAAGGGGCTCGTCCCTGTTTCCGGAGGAATT
TGCAAGCGGGGTCTTGCATGACGGGGAGGCAAACCCCCGTTCGCCGCAGTCCGGCCG
GCCCGAGACTCGAACCGGGGGTCCTGCGACTCAACCCTTGGAAAATAACCCTCCGGC
TACAGGGAGCGAGCCACTTAATGCTTTCGCTTTCCAGCCTAACCGCTTACGCCGCGCG
CGGCCAGTGGCCAAAAAAGCTAGCGCAGCAGCCGCCGCGCCTGGAAGGAAGCCAAA
AGGAGCGCTCCCCCGTTGTCTGACGTCGCACACCTGGGTTCGACACGCGGGCGGTAA
CCGCATGGATCACGGCGGACGGCCGGATCCGGGGTTCGAACCCCGGTCGTCCGCCAT
GATACCCTTGCGAATTTATCCACCAGACCACGGAAGAGTGCCCGCTTACAGGCTCTCC
TTTTGCACGGTCTAGAGCGTCAACGACTGCGCACGCCTCACCGGCCAGAGCGTCCCG
ACCATGGAGCACTTTTTGCCGCTGCGCAACATCTGGAACCGCGTCCGCGACTTTCCGC
GCGCCTCCACCACCGCCGCCGGCATCACCTGGATGTCCAGGTACATCTACGGATTAC
GGGCGCG
SEQ ID NO: 27 (STXC00124)
TGGTATGGCTTTTTCCCCGTATCCCCCCAGGTGTCTGCAGGCTCAAAGAG
CAGCGAGAAGCGTTCAGAGGAAAGCGATCCCGTGCCACCTTCCCCGTGCCCGGGCTG
TCCCCGCACGCTGCCGGCTCGGGGATGCGGGGGGAGCGCCGGACCGGAGCGGAGCC
CCGGGCGGCTCGCTGCTGCCCCCTAGCGGGGGAGGGACGTAATTACATCCCTGGGGG
CTTTGGGGGGGGGCTGTCCCTGATATCTATAACAAGAAAATATATATATAATAAGTT
ATCACGTAAGTAGAACATGAAATAACAATATAATTATCGTATGAGTTAAATCTTAAA
AGTCACGTAAAAGATAATCATGCGTCATTTTGACTCACGCGGTCGTTATAGTTCAAAA
TCAGTGACACTTACCGCATTGACAAGCACGCCTCACGGGAGCTCCAAGCGGCGACTG
AGATGTCCTAAATGCACAGCGACGGATTCGCGCTATTTAGAAAGAGAGAGCAATATT
TCAAGAATGCATGCGTCAATTTTACGCAGACTATCTTTCTAGGGTTAATCTAGCTGCA
TCAGGATCATATCGTCGGGTCTTTTTTCCGGCTCAGTCATCGCCCAAGCTGGCGCTAT
CTGGGCATCGGGGAGGAAGAAGCCCGTGCCTTTTCCCGCGAGGTTGAAGCGGCATGG
AAAGAGTTTGCCGAGGATGACTGCTGCTGCATTGACGTTGAGCGAAAACGCACGTTT
ACCATGATGATTCGGGAAGGTGTGGCCATGCACGCCTTTAACGGTGAACTGTTCGTTC
AGGCCACCTGGGATACCAGTTCGTCGCGGCTTTTCCGGACACAGTTCCGGATGGTCA
GCCCGAAGCGCATCAGCAACCCGAACAATACCGGCGACAGCCGGAACTGCCGTGCC
GGTGTGCAGATTAATGACAGCGGTGCGGCGCTGGGATATTACGTCAGCGAGGACGGG
TATCCTGGCTGGATGCCGCAGAAATGGACATGGATACCCCGTGAGTTACCCGGCGGG
CGCGCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGAT
AACTACGATACGGGAGGGCTTACCATCTGGCCCCAGTGCTGCAATGATACCGCGAGA
CCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCA
ACGATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCC
AGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTT
ATCAGGGTTATTGTCTCATGAGCGGATACATATTTGAATGTATTTAGAAAAATAAACA
AATAGGGGTTCCGCGCACATTTCCCCGAAAAGTGCCACCTAAATTGTAAGCGTTAAT
ATTTTGTTAAAATTCGCGTTAAATTTTTGTTAAATCAGCTCATTTTTTAACCAATAGGC
CGAAATCGGCAAAATCCCTTATAAATCAAAAGAATAGACCGAGATAGGGTTGAGTGT
TGTTCCAGTTTGGAACAAGAGTCCACTATTAAAGAACGTGGACTCCAACGTCAAAGG
GCGAAAAACCGTCTATCAGGGCGATGGCCCACTACGTGAACCATCACCCTAATCAAG
TTTTTTGGGGTCGAGGTGCCGTAAAGCACTAAATCGGAACCCTAAAGGGAGCCCCCG
ATTTAGAGCTTGACGGGGAAAGCCGGCGAACGTGGCGAGAAAGGAAGGGAAGAAAG
CGAAAGGAGCGGGCGCTAGGGCGCTGGCAAGTGTAGCGGTCACGCTGCGCGTAACC
ACCACACCCGCCGCGCTTAATGCGCCGCTACAGGGCGCGTCCCATTCGCCATTCAGG
CTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAG
TCACGACGTTGTAAAACGACGGCCAGTGAGCGCGCCTCGTTCATTCACGTTTTTGAAC
CCGTGGAGGACGGGCAGACTCGCGGTGCAAATGTGTTTTACAGCGTGATGGAGCAGA
TGAAGATGCTCGACACGCTGCAGAACACGCAGCTAGATTAACCCTAGAAAGATAATC
ATATTGTGACGTACGTTAAAGATAATCATGTGTAAAATTGACGCATGTGTTTTATCGG
TCTGTATATCGAGGTTTATTTATTAATTTGAATAGATATTAAGTTTTATTATATTTACA
CTTACATACTAATAATAAATTCAACAAACAATTTATTTATGTTTATTTATTTATTAAAA
AAAACAAAAACTCAAAATTTCTTCTATAAAGTAACAAAACTTTTATGAGGGACAGCC
CCCCCCCAAAGCCCCCAGGGATGTAATTACGTCCCTCCCCCGCTAGGGGGCAGCAGC
GAGCCGCCCGGGGCTCCGCTCCGGTCCGGCGCTCCCCCCGCATCCCCGAGCCGGCAG
CGTGCGGGGACAGCCCGGGCACGGGGAAGGTGGCACGGGATCGCTTTCCTCTGAACG
CTTCTCGCTGCTCTTTGAGCCTGCAGACACCTGGGGGGATACGGGGAAAAGGCCTCC
ACGGCCACTAGTCCATAGAGCCCACCGCATCCCCAGCATGCCTGCTATTGTCTTCCCA
ATCCTCCCCCTTGCTGTCCTGCCCCACCCCACCCCCTAGAATAGAATGACACCTACTC
AGACAATGCGATGCAATTTCCTCATTTTATTAGGAAAGGACAGTGGGAGTGGCACCT
TCCAGGGTCAAGGAAGGCACGGGGGAGGGGCAAACAACAGATGGCTGGCAACTAGA
AGGCACAGCTACATGGGGGTAGAGTCATAATCGTGCATCAGGATAGGGCGGTGGTGC
TGCAGCAGCGCGCGAATAAACTGCTGCCGCCGCCGCTCCGTCCTGCAGGAATACAAC
ATGGCAGTGGTCTCCTCAGCGATGATTCGCACCGCCCGCAGCATGAGACGCCTTGTCC
TCCGGGCACAGCAGCGCACCCTGATCTCACTTAAATCAGCACAGTAACTGCAGCACA
GCACCACAATATTGTTCAAAATCCCACAGTGCAAGGCGCTGTATCCAAAGCTCATGG
CGGGGACCACAGAACCCACGTGGCCATCATACCACAAGCGCAGGTAGATTAAGTGGC
GACCCCTCATAAACACGCTGGACATAAACATTACCTCTTTTGGCATGTTGTAATTCAC
CACCTCCCGGTACCATATAAACCTCTGATTAAACATGGCGCCATCCACCACCATCCTA
AACCAGCTGGCCAAAACCTGCCCGCCGGCTATGCACTGCAGGGAACCGGGACTGGAA
CAATGACAGTGGAGAGCCCAGGACTCGTAACCATGGATCATCATGCTCGTCATGATA
TCAATGTTGGCACAACACAGGCACACGTGCATACACTTCCTCAGGATTACAAGCTCCT
CCCGCGTCAGAACCATATCCCAGGGAACAACCCATTCCTGAATCAGCGTAAATCCCA
CACTGCAGGGAAGACCTCGCACGTAACTCACGTTGTGCATTGTCAAAGTGTTACATTC
GGGCAGCAGCGGATGATCCTCCAGTATGGTAGCGCGGGTCTCTGTCTCAAAAGGAGG
TAGGCGATCCCTACTGTACGGAGTGCGCCGAGACAACCGAGATCGTGTTGGTCGTAG
TGTCATGCCAAATGGAACGCCGGACGTAGTCATGGTTGTGGCCATATTATCATCGTGT
TTTTCAAAGGAAAACCACGTCCCCGTGGTTCGGGGGGCCTAGACGTTTTTTTAACCTC
GACTAAACACATGTAAAGCATGTGCACCGAGGCCCCAGATCAGATCCCATACAATGG
GGTACCTTCTGGGCATCCTTCAGCCCCTTGTTGAATACGCTTGAGGAGAGCCATTTGA
CTCTTTCCACAACTATCCAACTCACAACGTGGCACTGGGGTTGTGCCGCCTTTGCAGG
TGTATCTTATACACGTGGCTTTTGGCCGCAGAGGCACCTGTCGCCAGGTGGGGGGTTC
CGCTGCCTGCAAAGGGTCGCTACAGACGTTGTTTGTCTTCAAGAAGCTTCCAGAGGA
ACTGCTTCCTTCACGACATTCAACAGACCTTGCATTCCTTTGGCGAGAGGGGAAAGAC
CCCTAGGAATGCTCGTCAAGAAGACAGGGCCAGGTTTCCGGGCCCTCACATTGCCAA
AAGACGGCAATATGGTGGAAAATAACATATAGACAAACGCACACCGGCCTTATTCCA
AGCGGCTTCGGCCAGTAACGTTAGGGGGGGGGGAGGGAGAGGGGCTTAAAAATCAA
AGGGGTTCTGCCGCGCATCACTATGCGCCACTGGCAGGGACACGTTGCGATACTGGT
GTTTAGTGCTCCACTTAAACTCAGGCACAACCATCCGCGGCAGCTCGGTGAAGTTTTC
ACTCCACAGGCTGCGCACCATCACCAACGCGTTTAGCAGGTCGGGCGCCGATATCTT
GAAGTCGCAGTTGGGGCCTCCGCCCTGCGCGCGCGAGTTGCGATACACAGGGTTGCA
GCACTGGAACACTATCAGCGCCGGGTGGTGCACGCTGGCCAGCACGCTCTTGTCGGA
GATCAGATCCGCGTCCAGGTCCTCCGCGTTGCTCAGGGCGAACGGAGTCAACTTTGG
TAGCTGCCTTCCCAAAAAGGGTGCATGCCCAGGCTTTGAGTTGCACTCGCACCGTAGT
GGCATCAGAAGGTGACCGTGCCCGGTCTGGGCGTTAGGATACAGCGCCTGCATGAAA
GCCTTGATCTGCTTAAAAGCCACCTGAGCCTTTGCGCCTTCAGAGAAGAACATGCCGC
AAGACTTGCCGGAAAACTGATTGGCCGGACAGGCCGCGTCATGCACGCAGCACCTTG
CGTCGGTGTTGGAGATCTGCACCACATTTCGGCCCCACCGGTTCTTCACGATCTTGGC
CTTGCTAGACTGCTCCTTCAGCGCGCGCTGCCCGTTTTCGCTCGTCACATCCATTTCAA
TCACGTGCTCCTTATTTATCATAATGCTCCCGTGTAGACACTTAAGCTCGCCTTCGATC
TCAGCGCAGCGGTGCAGCCACAACGCGCAGCCCGTGGGCTCGTGGTGCTTGTAGGTT
ACCTCTGCAAACGACTGCAGGTACGCCTGCAGGAATCGCCCCATCATCGTCACAAAG
GTCTTGTTGCTGGTGAAGGTCAGCTGCAACCCGCGGTGCTCCTCGTTTAGCCAGGTCT
TGCATACGGCCGCCAGAGCTTCCACTTGGTCAGGCAGTAGCTTGAAGTTTGCCTTTAG
ATCGTTATCCACGTGGTACTTGTCCATCAACGCGCGCGCAGCCTCCATGCCCTTCTCC
CACGCAGACACGATCGGCAGGCTCAGCGGGTTTATCACCGTGCTTTCACTTTCCGCTT
CACTGGACTCTTCCTTTTCCTCTTGCGTCCGCATACCCCGCGCCACTGGGTCGTCTTCA
TTCAGCCGCCGCACCGTGCGCTTACCTCCCTTGCCGTGCTTGATTAGCACCGGTGGGT
TGCTGAAACCCACCATTTGTAGCGCCACATCTTCTCTTTCTTCCTCGCTGTCCACGATC
ACCTCTGGGGATGGCGGGCGCTCGGGCTTGGGAGAGGGGCGCTTCTTTTTCTTTTTGG
ACGCAATGGCCAAATCCGCCGTCGAGGTCGATGGCCGCGGGCTGGGTGTGCGCGGCA
CCAGCGCATCTTGTGACGAGTCTTCTTCGTCCTCGGACTCGAGACGCCGCCTCAGCCG
CTTTTTTGGGGGCGCGCGCTTGTCGTCATCGTCTTTGTAGTCGGGAGGCGGCGGCGAC
GGCGACGGGGACGACACGTCCTCCATGGTTGGTGGACGTCGCGCCGCACCGCGTCCG
CGCTCGGGGGTGGTTTCGCGCTGCTCCTCTTCCCGACTGGCCATGGTGGCCGAGGATA
ACTTCGTATATGGTTTCTTATACGAAGTTATGATCCAGACATGATAAGATACATTGAT
GAGTTTGGACAAACCACAACTAGAATGCAGTGAAAAAAATGCTTTATTTGTGAAATT
TGTGATGCTATTGCTTTATTTGTAACCATTATAAGCTGCAATAAACAAGTTAACAACA
ACAATTGCATTCATTTTATGTTTCAGGTTCAGGGGGAGGTGTGGGAGGTTTTTTAAAG
CAAGTAAAACCTCTACAAATGTGGTATGGCTGATTATGATCCTCTAGAGTCGCAGATC
TGCTACGTATCAAGCTGTGGCAGGGAAACCCTCTGCCTCCCCCGTGATGTAATACTTT
TGCAAGGAATGCGATGAAGTAGAGCCCGCAGTGGCCAAGTGGCTTTGGTCCGTCTCC
TCCACGGATGCCCCTCCACGGCTAGTGGGCGCATGTAGGCGGTGGGCGTCCGCCGCC
TCCAGCAGCAGGTCATAGAGGGGCACCACGTTCTTGCACTTCATGCTGTACAGATGCT
CCATGCCTTTGTTACTCATGTGTCGGATGTGGGAGAGGATGAGGAGGAGCTGGGCCA
GCCGCTGGTGCTGCTGCTGCAGGGTCAGGCCTGCCTTGGCCATCAGGTGGATCAAAG
TGTCTGTGATCTTGTCCAGGACTCGGTGGATATGGTCCTTCTCTTCCAGAGACTTCAG
GGTGCTGGACAGAAATGTGTACACTCCAGAATTAAGCAAAATAATAGATTTGAGGCA
CACAAACTCCTCTCCCTGCAGATTCATCATGCGGAACCGAGATGATGTAGCCAGCAG
CATGTCGAAGATCTCCACCATGCCCTCTACACATTTTCCCTGGTTCCTGTCCAAGAGC
AAGTTAGGAGCAAACAGTAGCTTCACTGGGTGCTCCATGGAGCGCCAGACGAGACCA
ATCATCAGGATCTCTAGCCAGGCACATTCTAGAAGGTGGACCTGATCATGGAGGGTC
AAATCCACAAAGCCTGGCACCCTCTTCGCCCAGTTGATCATGTGAACCAGCTCCCTGT
CTGCCAGGTTGGTCAGTAAGCCCATCATCGAAGCTTCACTGAAGGGTCTGGTAGGAT
CATACTCGGAATAGAGTATGGGGGGCTCAGCATCCAACAAGGCACTGACCATCTGGT
CGGCCGTCAGGGACAAGGCCAGGCTGTTCTTCTTAGAGCGTTTGATCATGAGCGGGC
TTGGCCAAAGGTTGGCAGCTCTCATGTCTCCAGCAGATGGCTCGAGATCGCCATCTTC
CAGCAGGCGCACCATTGCCCCTGTTTCACTATCCAGGTTACGGATATAGTTCATGACA
ATATTTACATTGGTCCAGCCACCAGCTTGCATGATCTCCGGTATTGAAACTCCAGCGC
GGGCCATATCTCGCGCGGCTCCGACACGGGCACTGTGTCCAGACCAGGCCAGGTATC
TCTGACCAGAGTCATCCTAAAATACACAAACAATTAGAATCAGTAGTTTAACACATT
ATACACTTAAAAATTTTATATTTACCTTAGCGCCGTAAATCAATCGATGAGTTGCTTC
AAAAATCCCTTCCAGGGCGCGAGTTGATAGCTGGCTGGTGGCAGATGGCGCGGCAAC
ACCATTTTTTCTGACCCGGCAAAACAGGTAGTTATTCGGATCATCAGCTACACCAGAG
ACGGAAATCCATCGCTCGACCAGTTTAGTGACTCCCAGGCTAAGTGCCTTCTCTACAC
CTGCGGTGCTAACCAGCGTTTTCGTTCTGCCAATATGGATTAACATTCTCCCACCGTC
AGTACGTGAGATATCTTTAACCCTGATCCTGGCAATTTCGGCTATACGTAACAGGGTG
TTATAAGCAATCCCCAGAAATGCCAGATTACGTATATCCTGGCAGCGATCGCTATTTT
CCATGAGTGAACGGACTTGGTCGAAATCAGTGCGTTCGAACGCTAGAGCCTGTTTTG
CACGTTCACCGGCATCAACGTTTTCTTTTCGGATCCGCCGCATAACCAGTGAAACAGC
ATTGCTGTCACTTGGTCGTGGCAGCCCGGACCGACGATGAAGCATGTTTAGCTGGCCC
AAATGTTGCTGGATAGTTTTTACTGCCAGACCGCGCGCTTGAAGATATAGAAGATAA
TCGCGAACATCTTCAGGTTCTGCGGGAAACCATTTCCGGTTATTCAACTTGCACCATG
CCGCCCACGACCGGCAAACGGACAGAAGCATTTTCCAGGTATGCTCAGAAAACGCCT
GGCGATCCCTGAACATGTCCATCAGGTTCTTGCGAACCTCATCACTCGTTGCATCGAC
CGGTAATGCAGGCAAATTTTGGTGTACGGTCAGTAAATTGGACATGGTGGCTACGTA
ATAACTTCGTATATGGTTTCTTATACGAAGTTATGCGGCCGCTTTACGAGGGTAGGAA
GTGGTACGGAAAGTTGGTATAAGACAAAAGTGTTGTGGAATTGCTCCAGGCGATCTG
ACGGTTCACTAAACGAGCTCTGCTTTTATAGGCGCCCACCGTACACGCCTAAAGCTTA
TACGTTCTCTATCACTGATAGGGAGTAAACTGGATATACGTTCTCTATCACTGATAGG
GAGTAAACTGTAGATACGTTCTCTATCACTGATAGGGAGTAAACTGGTCATACGTTCT
CTATCACTGATAGGGAGTAAACTCCTTATACGTTCTCTATCACTGATAGGGAGTAAAG
TCTGCATACGTTCTCTATCACTGATAGGGAGTAAACTCTTCATACGTTCTCTATCACTG
ATAGGGAGTAAACTCGCGGCCGCAGAGAAATGTTCTGGCACCTGCACTTGCACTGGG
GACAGCCTATTTTGCTAGTTTGTTTTGTTTCGTTTTGTTTTGATGGAGAGCGTATGTTA
GTACTATCGATTCACACAAAAAACCAACACACAGATGTAATGAAAATAAAGATATTT
TATTGGATCTGCGATCGCTCCGGTGCCCGTCAGTGGGCAGAGCGCACATCGCCCACA
GTCCCCGAGAAGTTGGGGGGAGGGGTCGGCAATTGAACGGGTGCCTAGAGAAGGTG
GCGCGGGGTAAACTGGGAAAGTGATGTCGTGTACTGGCTCCGCCTTTTTCCCGAGGG
TGGGGGAGAACCGTATGTAAGTGCAGTAGTCGCCGTGAACGTTCTTTTTCGCAACGG
GTTTGCCGCCAGAACACAGCTGAAGCTTCGAGGGGCTCGCATCTCTCCTTCACGCGCC
CGCCGCCCTACCTGAGGCCGCCATCCACGCCGGTTGAGTCGCGTTCTGCCGCCTCCCG
CCTGTGGTGCCTCCTGAACTGCGTCCGCCGTCTAGGTAAGTTTAAAGCTCAGGTCGAG
ACCGGGCCTTTGTCCGGCGCTCCCTTGGAGCCTACCTAGACTCAGCCGGCTCTCCACG
CTTTGCCTGACCCTGCTTGCTCAACTCTACGTCTTTGTTTCGTTTTCTGTTCTGCGCCGT
TACAGATCCAAGCTGTGACCGGCGCCTACGCTAGCGGATCCGCCGCCACCATGTCTA
GACTGGACAAGAGCAAAGTCATAAACTCTGCTCTGGAATTACTCAATGGAGTCGGTA
TCGAAGGCCTGACGACAAGGAAACTCGCTCAAAAGCTGGGAGTTGAGCAGCCTACCC
TGTACTGGCACGTGAAGAACAAGCGGGCCCTGCTCGATGCCCTGCCAATCGAGATGC
TGGACAGGCATCATACCCACTCCTGCCCCCTGGAAGGCGAGTCATGGCAAGACTTTC
TGCGGAACAACGCCAAGTCATACCGCTGTGCTCTTCTCTCACATCGCGACGGGGCTA
AAGTGCATCTCGGCACCCGCCCAACAGAGAAACAGTACGAAACCCTGGAAAATCAG
CTCGCGTTCCTGTGTCAGCAAGGCTTCTCCCTGGAGAACGCACTGTACGCTCTGTCCG
CCGTGGGCCACTTTACACTGGGCTGCGTATTGGAGGAACAGGAGCATCAAGTAGCAA
AAGAGGAAAGAGAGACACCTACCACCGATTCTATGCCCCCACTTCTGAAACAAGCAA
TTGAGCTGTTCGACCGGCAGGGAGCCGAACCTGCCTTCCTTTTCGGCCTGGAACTAAT
CATATGTGGCCTGGAGAAACAGCTAAAGTGCGAAAGCGGCGGGCCGACCGACGCCC
TTGACGATTTTGACTTAGACATGCTCCCAGCCGATGCCCTTGACGACTTTGACCTTGA
TATGCTGCCTGCTGACGCTCTTGACGATTTTGACCTTGACATGCTCCCCGGGTGAACC
GGTCGCTGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGC
GGTGGGCTCTATGGCTTCTGAGGCGGAAAGAACCAGCTGGGGCTCGACTAGAGCTTG
CGGAACCCTTAGAGGGCCTATTTCCCATGATTCCTTCATATTTGCATATACGATACAA
GGCTGTTAGAGAGATAATTAGAATTAATTTGACTGTAAACACAAAGATATTAGTACA
AAATAATAACTTCGTATAATGTATGCTATACGAAGTTATCAGACATGATAAGATACA
TTGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGGTAC
CTCAAGCGCCGGGTTTTCGCGTCATGCACCACGTCCGTGGTAGAACTAGTATTATGCC
CAGTACATGACCTTATGGGACTTTCCTACTTGGCAGTACATCTACGTATTAGTCATCG
CTATTACCATGGTGATGCGGTTTTGGCAGTACATCAATGGGCGTGGATAGCGGTTTGA
CTCACGGGGATTTCCAAGTCTCCACCCCATTGACGTCAATGGGAGTTTGTTTTGGCAC
CAAAATCAACGGGACTTTCCAAAATGTCGTAACAACTCCGCCCCATTGACGCAAATG
GGCGGTAGGCGTGTACGGTGGGAGGTTTATATAAGCAGAGCTCGTTTAGTGAACCGT
CAGATCGCCTGGAGACGCCATCCACGCTGTTTTGACCTCCATAGAAGAGAATTCGCC
GCCACCATGACAGAATATAAACCTACTGTCAGACTGGCAACTCGAGACGACGTCCCT
AGGGCCGTGAGAACATTGGCTGCCGCTTTCGCGGATTATCCCGCTACACGCCACACA
GTTGATCCTGATAGACATATTGAACGGGTTACAGAATTGCAAGAACTTTTTTTGACCA
GGGTAGGATTGGACATCGGTAAAGTTTGGGTCGCCGACGACGGGGCTGCAGTGGCAG
TGTGGACGACTCCGGAGAGCGTTGAGGCCGGGGCTGTATTTGCAGAAATTGGTCCCC
GAATGGCTGAGCTTAGTGGCTCTCGTCTCGCGGCTCAGCAACAAATGGAAGGACTCC
TCGCCCCTCACCGCCCTAAAGAACCAGCTTGGTTCCTCGCTACTGTGGGCGTTAGCCC
CGATCATCAGGGAAAGGGCCTTGGTTCCGCGGTGGTATTGCCCGGAGTAGAAGCCGC
AGAACGAGCCGGAGTGCCAGCCTTTCTTGAAACGTCAGCGCCAAGGAATTTGCCCTT
CTATGAACGGCTCGGATTTACAGTTACTGCTGACGTTGAAGTACCCGAGGGCCCACG
GACGTGGTGCATGACGCGAAAACCCGGCGCTTGAACCGGTCGCTGATCAGCCTCGAC
TGTGCCTTCTAGTTGCCAGCCATCTGTTGTTTGCCCCTCCCCCGTGCCTTCCTTGACCC
TGGAAGGTGCCACTCCCACTGTCCTTTCCTAATAAAATGAGGAAATTGCATCGCATTG
TCTGAGTAGGTGTCATTCTATTCTGGGGGGTGGGGTGGGGCAGGACAGCAAGGGGGA
GGATTGGGAAGACAATAGCAGGCATGCTGGGGATGCGGTGGGCTCTATGGCTTCTGA
GGCGGAAAGAACCAGCTGGGGCTCGACTAGAGCTTGCGGAACCCTTAGGTTGGGAA
AAGCGCTCCCCTACCCATAACTTCGTATAATGTATGCTATACGAAGTTATTTTGCAGT
TTTAAAATTATGTTTTAAAATGGACTATCATATGCTTACCGTAACTTGAAAGTATTTC
GATTTCTTGGCTTTATATATCTTGTGGAAAGGACGAAACACCGGGCACTCTTCCGTGA
TCTGGTGGATAAATTCGCAAGGGTATCATGGCGGACGACCGGGATTCGAACCCCGGA
TCCGGCCGTCCGCCGTGATCCATGCGGTTACCGCCCGCGTGTCGAACCCAGGTGTGCG
ACGTCAGACAACGGGGGAGCGCTCCTTTTTGGGCCCAT
SEQ ID NO: 28 (STXC0125)
TGGTATGGCTTTTTCCCCGTATCCCCCCAGGTGTCTGCAGGCTCAAAGAG
CAGCGAGAAGCGTTCAGAGGAAAGCGATCCCGTGCCACCTTCCCCGTGCCCGGGCTG
TCCCCGCACGCTGCCGGCTCGGGGATGCGGGGGGAGCGCCGGACCGGAGCGGAGCC
CCGGGCGGCTCGCTGCTGCCCCCTAGCGGGGGAGGGACGTAATTACATCCCTGGGGG
CTTTGGGGGGGGGCTGTCCCTGATATCTATAACAAGAAAATATATATATAATAAGTT
ATCACGTAAGTAGAACATGAAATAACAATATAATTATCGTATGAGTTAAATCTTAAA
AGTCACGTAAAAGATAATCATGCGTCATTTTGACTCACGCGGTCGTTATAGTTCAAAA
TCAGTGACACTTACCGCATTGACAAGCACGCCTCACGGGAGCTCCAAGCGGCGACTG
AGATGTCCTAAATGCACAGCGACGGATTCGCGCTATTTAGAAAGAGAGAGCAATATT
TCAAGAATGCATGCGTCAATTTTACGCAGACTATCTTTCTAGGGTTAATCTAGCTGCA
TCAGGATCATATCGTCGGGTCTTTTTTCCGGCTCAGTCATCGCCCAAGCTGGCGCTAT
CTGGGCATCGGGGAGGAAGAAGCCCGTGCCTTTTCCCGCGAGGTTGAAGCGGCATGG
AAAGAGTTTGCCGAGGATGACTGCTGCTGCATTGACGTTGAGCGAAAACGCACGTTT
ACCATGATGATTCGGGAAGGTGTGGCCATGCACGCCTTTAACGGTGAACTGTTCGTTC
AGGCCACCTGGGATACCAGTTCGTCGCGGCTTTTCCGGACACAGTTCCGGATGGTCA
GCCCGAAGCGCATCAGCAACCCGAACAATACCGGCGACAGCCGGAACTGCCGTGCC
GGTGTGCAGATTAATGACAGCGGTGCGGCGCTGGGATATTACGTCAGCGAGGACGGG
TATCCTGGCTGGATGCCGCAGAAATGGACATGGATACCCCGTGAGTTACCCGGCGGG
CGCGCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGAT
AACTACGATACGGGAGGGCTTACCATCTGGCCCCAGTGCTGCAATGATACCGCGAGA
CCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCA
ACGATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCC
AGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTT
ATCAGGGTTATTGTCTCATGAGCGGATACATATTTGAATGTATTTAGAAAAATAAACA
AATAGGGGTTCCGCGCACATTTCCCCGAAAAGTGCCACCTAAATTGTAAGCGTTAAT
ATTTTGTTAAAATTCGCGTTAAATTTTTGTTAAATCAGCTCATTTTTTAACCAATAGGC
CGAAATCGGCAAAATCCCTTATAAATCAAAAGAATAGACCGAGATAGGGTTGAGTGT
TGTTCCAGTTTGGAACAAGAGTCCACTATTAAAGAACGTGGACTCCAACGTCAAAGG
GCGAAAAACCGTCTATCAGGGCGATGGCCCACTACGTGAACCATCACCCTAATCAAG
TTTTTTGGGGTCGAGGTGCCGTAAAGCACTAAATCGGAACCCTAAAGGGAGCCCCCG
ATTTAGAGCTTGACGGGGAAAGCCGGCGAACGTGGCGAGAAAGGAAGGGAAGAAAG
CGAAAGGAGCGGGCGCTAGGGCGCTGGCAAGTGTAGCGGTCACGCTGCGCGTAACC
ACCACACCCGCCGCGCTTAATGCGCCGCTACAGGGCGCGTCCCATTCGCCATTCAGG
CTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAG
TCACGACGTTGTAAAACGACGGCCAGTGAGCGCGCCTCGTTCATTCACGTTTTTGAAC
CCGTGGAGGACGGGCAGACTCGCGGTGCAAATGTGTTTTACAGCGTGATGGAGCAGA
TGAAGATGCTCGACACGCTGCAGAACACGCAGCTAGATTAACCCTAGAAAGATAATC
ATATTGTGACGTACGTTAAAGATAATCATGTGTAAAATTGACGCATGTGTTTTATCGG
TCTGTATATCGAGGTTTATTTATTAATTTGAATAGATATTAAGTTTTATTATATTTACA
CTTACATACTAATAATAAATTCAACAAACAATTTATTTATGTTTATTTATTTATTAAAA
AAAACAAAAACTCAAAATTTCTTCTATAAAGTAACAAAACTTTTATGAGGGACAGCC
CCCCCCCAAAGCCCCCAGGGATGTAATTACGTCCCTCCCCCGCTAGGGGGCAGCAGC
GAGCCGCCCGGGGCTCCGCTCCGGTCCGGCGCTCCCCCCGCATCCCCGAGCCGGCAG
CGTGCGGGGACAGCCCGGGCACGGGGAAGGTGGCACGGGATCGCTTTCCTCTGAACG
CTTCTCGCTGCTCTTTGAGCCTGCAGACACCTGGGGGGATACGGGGAAAAGGCCTCC
ACGGCCACTAGTCCATAGAGCCCACCGCATCCCCAGCATGCCTGCTATTGTCTTCCCA
ATCCTCCCCCTTGCTGTCCTGCCCCACCCCACCCCCTAGAATAGAATGACACCTACTC
AGACAATGCGATGCAATTTCCTCATTTTATTAGGAAAGGACAGTGGGAGTGGCACCT
TCCAGGGTCAAGGAAGGCACGGGGGAGGGGCAAACAACAGATGGCTGGCAACTAGA
AGGCACAGCTACATGGGGGTAGAGTCATAATCGTGCATCAGGATAGGGCGGTGGTGC
TGCAGCAGCGCGCGAATAAACTGCTGCCGCCGCCGCTCCGTCCTGCAGGAATACAAC
ATGGCAGTGGTCTCCTCAGCGATGATTCGCACCGCCCGCAGCATGAGACGCCTTGTCC
TCCGGGCACAGCAGCGCACCCTGATCTCACTTAAATCAGCACAGTAACTGCAGCACA
GCACCACAATATTGTTCAAAATCCCACAGTGCAAGGCGCTGTATCCAAAGCTCATGG
CGGGGACCACAGAACCCACGTGGCCATCATACCACAAGCGCAGGTAGATTAAGTGGC
GACCCCTCATAAACACGCTGGACATAAACATTACCTCTTTTGGCATGTTGTAATTCAC
CACCTCCCGGTACCATATAAACCTCTGATTAAACATGGCGCCATCCACCACCATCCTA
AACCAGCTGGCCAAAACCTGCCCGCCGGCTATGCACTGCAGGGAACCGGGACTGGAA
CAATGACAGTGGAGAGCCCAGGACTCGTAACCATGGATCATCATGCTCGTCATGATA
TCAATGTTGGCACAACACAGGCACACGTGCATACACTTCCTCAGGATTACAAGCTCCT
CCCGCGTCAGAACCATATCCCAGGGAACAACCCATTCCTGAATCAGCGTAAATCCCA
CACTGCAGGGAAGACCTCGCACGTAACTCACGTTGTGCATTGTCAAAGTGTTACATTC
GGGCAGCAGCGGATGATCCTCCAGTATGGTAGCGCGGGTCTCTGTCTCAAAAGGAGG
TAGGCGATCCCTACTGTACGGAGTGCGCCGAGACAACCGAGATCGTGTTGGTCGTAG
TGTCATGCCAAATGGAACGCCGGACGTAGTCATGGTTGTGGCCATATTATCATCGTGT
TTTTCAAAGGAAAACCACGTCCCCGTGGTTCGGGGGGCCTAGACGTTTTTTTAACCTC
GACTAAACACATGTAAAGCATGTGCACCGAGGCCCCAGATCAGATCCCATACAATGG
GGTACCTTCTGGGCATCCTTCAGCCCCTTGTTGAATACGCTTGAGGAGAGCCATTTGA
CTCTTTCCACAACTATCCAACTCACAACGTGGCACTGGGGTTGTGCCGCCTTTGCAGG
TGTATCTTATACACGTGGCTTTTGGCCGCAGAGGCACCTGTCGCCAGGTGGGGGGTTC
CGCTGCCTGCAAAGGGTCGCTACAGACGTTGTTTGTCTTCAAGAAGCTTCCAGAGGA
ACTGCTTCCTTCACGACATTCAACAGACCTTGCATTCCTTTGGCGAGAGGGGAAAGAC
CCCTAGGAATGCTCGTCAAGAAGACAGGGCCAGGTTTCCGGGCCCTCACATTGCCAA
AAGACGGCAATATGGTGGAAAATAACATATAGACAAACGCACACCGGCCTTATTCCA
AGCGGCTTCGGCCAGTAACGTTAGGGGGGGGGGGGGAGAGGGGCTTAAAAATCAA
AGGGGTTCTGCCGCGCATCACTATGCGCCACTGGCAGGGACACGTTGCGATACTGGT
GTTTAGTGCTCCACTTAAACTCAGGCACAACCATCCGCGGCAGCTCGGTGAAGTTTTC
ACTCCACAGGCTGCGCACCATCACCAACGCGTTTAGCAGGTCGGGCGCCGATATCTT
GAAGTCGCAGTTGGGGCCTCCGCCCTGCGCGCGCGAGTTGCGATACACAGGGTTGCA
GCACTGGAACACTATCAGCGCCGGGTGGTGCACGCTGGCCAGCACGCTCTTGTCGGA
GATCAGATCCGCGTCCAGGTCCTCCGCGTTGCTCAGGGCGAACGGAGTCAACTTTGG
TAGCTGCCTTCCCAAAAAGGGTGCATGCCCAGGCTTTGAGTTGCACTCGCACCGTAGT
GGCATCAGAAGGTGACCGTGCCCGGTCTGGGCGTTAGGATACAGCGCCTGCATGAAA
GCCTTGATCTGCTTAAAAGCCACCTGAGCCTTTGCGCCTTCAGAGAAGAACATGCCGC
AAGACTTGCCGGAAAACTGATTGGCCGGACAGGCCGCGTCATGCACGCAGCACCTTG
CGTCGGTGTTGGAGATCTGCACCACATTTCGGCCCCACCGGTTCTTCACGATCTTGGC
CTTGCTAGACTGCTCCTTCAGCGCGCGCTGCCCGTTTTCGCTCGTCACATCCATTTCAA
TCACGTGCTCCTTATTTATCATAATGCTCCCGTGTAGACACTTAAGCTCGCCTTCGATC
TCAGCGCAGCGGTGCAGCCACAACGCGCAGCCCGTGGGCTCGTGGTGCTTGTAGGTT
ACCTCTGCAAACGACTGCAGGTACGCCTGCAGGAATCGCCCCATCATCGTCACAAAG
GTCTTGTTGCTGGTGAAGGTCAGCTGCAACCCGCGGTGCTCCTCGTTTAGCCAGGTCT
TGCATACGGCCGCCAGAGCTTCCACTTGGTCAGGCAGTAGCTTGAAGTTTGCCTTTAG
ATCGTTATCCACGTGGTACTTGTCCATCAACGCGCGCGCAGCCTCCATGCCCTTCTCC
CACGCAGACACGATCGGCAGGCTCAGCGGGTTTATCACCGTGCTTTCACTTTCCGCTT
CACTGGACTCTTCCTTTTCCTCTTGCGTCCGCATACCCCGCGCCACTGGGTCGTCTTCA
TTCAGCCGCCGCACCGTGCGCTTACCTCCCTTGCCGTGCTTGATTAGCACCGGTGGGT
TGCTGAAACCCACCATTTGTAGCGCCACATCTTCTCTTTCTTCCTCGCTGTCCACGATC
ACCTCTGGGGATGGCGGGCGCTCGGGCTTGGGAGAGGGGCGCTTCTTTTTCTTTTTGG
ACGCAATGGCCAAATCCGCCGTCGAGGTCGATGGCCGCGGGCTGGGTGTGCGCGGCA
CCAGCGCATCTTGTGACGAGTCTTCTTCGTCCTCGGACTCGAGACGCCGCCTCAGCCG
CTTTTTTGGGGGCGCGCGCTTGTCGTCATCGTCTTTGTAGTCGGGAGGCGGCGGCGAC
GGCGACGGGGACGACACGTCCTCCATGGTTGGTGGACGTCGCGCCGCACCGCGTCCG
CGCTCGGGGGTGGTTTCGCGCTGCTCCTCTTCCCGACTGGCCATGGTGGCCGAGGATA
ACTTCGTATATGGTTTCTTATACGAAGTTATGATCCAGACATGATAAGATACATTGAT
GAGTTTGGACAAACCACAACTAGAATGCAGTGAAAAAAATGCTTTATTTGTGAAATT
TGTGATGCTATTGCTTTATTTGTAACCATTATAAGCTGCAATAAACAAGTTAACAACA
ACAATTGCATTCATTTTATGTTTCAGGTTCAGGGGGAGGTGTGGGAGGTTTTTTAAAG
CAAGTAAAACCTCTACAAATGTGGTATGGCTGATTATGATCCTCTAGAGTCGCAGATC
TGCTACGTATCAAGCTGTGGCAGGGAAACCCTCTGCCTCCCCCGTGATGTAATACTTT
TGCAAGGAATGCGATGAAGTAGAGCCCGCAGTGGCCAAGTGGCTTTGGTCCGTCTCC
TCCACGGATGCCCCTCCACGGCTAGTGGGCGCATGTAGGCGGTGGGCGTCCGCCGCC
TCCAGCAGCAGGTCATAGAGGGGCACCACGTTCTTGCACTTCATGCTGTACAGATGCT
CCATGCCTTTGTTACTCATGTGTCGGATGTGGGAGAGGATGAGGAGGAGCTGGGCCA
GCCGCTGGTGCTGCTGCTGCAGGGTCAGGCCTGCCTTGGCCATCAGGTGGATCAAAG
TGTCTGTGATCTTGTCCAGGACTCGGTGGATATGGTCCTTCTCTTCCAGAGACTTCAG
GGTGCTGGACAGAAATGTGTACACTCCAGAATTAAGCAAAATAATAGATTTGAGGCA
CACAAACTCCTCTCCCTGCAGATTCATCATGCGGAACCGAGATGATGTAGCCAGCAG
CATGTCGAAGATCTCCACCATGCCCTCTACACATTTTCCCTGGTTCCTGTCCAAGAGC
AAGTTAGGAGCAAACAGTAGCTTCACTGGGTGCTCCATGGAGCGCCAGACGAGACCA
ATCATCAGGATCTCTAGCCAGGCACATTCTAGAAGGTGGACCTGATCATGGAGGGTC
AAATCCACAAAGCCTGGCACCCTCTTCGCCCAGTTGATCATGTGAACCAGCTCCCTGT
CTGCCAGGTTGGTCAGTAAGCCCATCATCGAAGCTTCACTGAAGGGTCTGGTAGGAT
CATACTCGGAATAGAGTATGGGGGGCTCAGCATCCAACAAGGCACTGACCATCTGGT
CGGCCGTCAGGGACAAGGCCAGGCTGTTCTTCTTAGAGCGTTTGATCATGAGCGGGC
TTGGCCAAAGGTTGGCAGCTCTCATGTCTCCAGCAGATGGCTCGAGATCGCCATCTTC
CAGCAGGCGCACCATTGCCCCTGTTTCACTATCCAGGTTACGGATATAGTTCATGACA
ATATTTACATTGGTCCAGCCACCAGCTTGCATGATCTCCGGTATTGAAACTCCAGCGC
GGGCCATATCTCGCGCGGCTCCGACACGGGCACTGTGTCCAGACCAGGCCAGGTATC
TCTGACCAGAGTCATCCTAAAATACACAAACAATTAGAATCAGTAGTTTAACACATT
ATACACTTAAAAATTTTATATTTACCTTAGCGCCGTAAATCAATCGATGAGTTGCTTC
AAAAATCCCTTCCAGGGCGCGAGTTGATAGCTGGCTGGTGGCAGATGGCGCGGCAAC
ACCATTTTTTCTGACCCGGCAAAACAGGTAGTTATTCGGATCATCAGCTACACCAGAG
ACGGAAATCCATCGCTCGACCAGTTTAGTGACTCCCAGGCTAAGTGCCTTCTCTACAC
CTGCGGTGCTAACCAGCGTTTTCGTTCTGCCAATATGGATTAACATTCTCCCACCGTC
AGTACGTGAGATATCTTTAACCCTGATCCTGGCAATTTCGGCTATACGTAACAGGGTG
TTATAAGCAATCCCCAGAAATGCCAGATTACGTATATCCTGGCAGCGATCGCTATTTT
CCATGAGTGAACGGACTTGGTCGAAATCAGTGCGTTCGAACGCTAGAGCCTGTTTTG
CACGTTCACCGGCATCAACGTTTTCTTTTCGGATCCGCCGCATAACCAGTGAAACAGC
ATTGCTGTCACTTGGTCGTGGCAGCCCGGACCGACGATGAAGCATGTTTAGCTGGCCC
AAATGTTGCTGGATAGTTTTTACTGCCAGACCGCGCGCTTGAAGATATAGAAGATAA
TCGCGAACATCTTCAGGTTCTGCGGGAAACCATTTCCGGTTATTCAACTTGCACCATG
CCGCCCACGACCGGCAAACGGACAGAAGCATTTTCCAGGTATGCTCAGAAAACGCCT
GGCGATCCCTGAACATGTCCATCAGGTTCTTGCGAACCTCATCACTCGTTGCATCGAC
CGGTAATGCAGGCAAATTTTGGTGTACGGTCAGTAAATTGGACATGGTGGCTACGTA
ATAACTTCGTATATGGTTTCTTATACGAAGTTATGCGGCCGCTTTACGAGGGTAGGAA
GTGGTACGGAAAGTTGGTATAAGACAAAAGTGTTGTGGAATTGCTCCAGGCGATCTG
ACGGTTCACTAAACGAGCTCTGCTTTTATAGGCGCCCACCGTACACGCCTAAAGCTTA
TACGTTCTCTATCACTGATAGGGAGTAAACTGGATATACGTTCTCTATCACTGATAGG
GAGTAAACTGTAGATACGTTCTCTATCACTGATAGGGAGTAAACTGGTCATACGTTCT
CTATCACTGATAGGGAGTAAACTCCTTATACGTTCTCTATCACTGATAGGGAGTAAAG
TCTGCATACGTTCTCTATCACTGATAGGGAGTAAACTCTTCATACGTTCTCTATCACTG
ATAGGGAGTAAACTCGCGGCCGCAGAGAAATGTTCTGGCACCTGCACTTGCACTGGG
GACAGCCTATTTTGCTAGTTTGTTTTGTTTCGTTTTGTTTTGATGGAGAGCGTATGTTA
GTACTATCGATTCACACAAAAAACCAACACACAGATGTAATGAAAATAAAGATATTT
TATTGGATCTGCGATCGCTCCGGTGCCCGTCAGTGGGCAGAGCGCACATCGCCCACA
GTCCCCGAGAAGTTGGGGGGAGGGGTCGGCAATTGAACGGGTGCCTAGAGAAGGTG
GCGCGGGGTAAACTGGGAAAGTGATGTCGTGTACTGGCTCCGCCTTTTTCCCGAGGG
TGGGGGAGAACCGTATGTAAGTGCAGTAGTCGCCGTGAACGTTCTTTTTCGCAACGG
GTTTGCCGCCAGAACACAGCTGAAGCTTCGAGGGGCTCGCATCTCTCCTTCACGCGCC
CGCCGCCCTACCTGAGGCCGCCATCCACGCCGGTTGAGTCGCGTTCTGCCGCCTCCCG
CCTGTGGTGCCTCCTGAACTGCGTCCGCCGTCTAGGTAAGTTTAAAGCTCAGGTCGAG
ACCGGGCCTTTGTCCGGCGCTCCCTTGGAGCCTACCTAGACTCAGCCGGCTCTCCACG
CTTTGCCTGACCCTGCTTGCTCAACTCTACGTCTTTGTTTCGTTTTCTGTTCTGCGCCGT
TACAGATCCAAGCTGTGACCGGCGCCTACGCTAGCGGATCCGCCGCCACCATGTCTA
GACTGGACAAGAGCAAAGTCATAAACTCTGCTCTGGAATTACTCAATGGAGTCGGTA
TCGAAGGCCTGACGACAAGGAAACTCGCTCAAAAGCTGGGAGTTGAGCAGCCTACCC
TGTACTGGCACGTGAAGAACAAGCGGGCCCTGCTCGATGCCCTGCCAATCGAGATGC
TGGACAGGCATCATACCCACTCCTGCCCCCTGGAAGGCGAGTCATGGCAAGACTTTC
TGCGGAACAACGCCAAGTCATACCGCTGTGCTCTTCTCTCACATCGCGACGGGGCTA
AAGTGCATCTCGGCACCCGCCCAACAGAGAAACAGTACGAAACCCTGGAAAATCAG
CTCGCGTTCCTGTGTCAGCAAGGCTTCTCCCTGGAGAACGCACTGTACGCTCTGTCCG
CCGTGGGCCACTTTACACTGGGCTGCGTATTGGAGGAACAGGAGCATCAAGTAGCAA
AAGAGGAAAGAGAGACACCTACCACCGATTCTATGCCCCCACTTCTGAAACAAGCAA
TTGAGCTGTTCGACCGGCAGGGAGCCGAACCTGCCTTCCTTTTCGGCCTGGAACTAAT
CATATGTGGCCTGGAGAAACAGCTAAAGTGCGAAAGCGGCGGGCCGACCGACGCCC
TTGACGATTTTGACTTAGACATGCTCCCAGCCGATGCCCTTGACGACTTTGACCTTGA
TATGCTGCCTGCTGACGCTCTTGACGATTTTGACCTTGACATGCTCCCCGGGTGAACC
GGTCGCTGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGC
GGTGGGCTCTATGGCTTCTGAGGCGGAAAGAACCAGCTGGGGCTCGACTAGAGCTTG
CGGAACCCTTAGAGGGCCTATTTCCCATGATTCCTTCATATTTGCATATACGATACAA
GGCTGTTAGAGAGATAATTAGAATTAATTTGACTGTAAACACAAAGATATTAGTACA
AAATAATAACTTCGTATAATGTATGCTATACGAAGTTATCAGACATGATAAGATACA
TTGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGGTAC
CTCAAGCGCCGGGTTTTCGCGTCATGCACCACGTCCGTGGTCAACCCTCCCACACGTA
ACCAGATGGGAGAAGCTCTCGTATTCCAACAGCTGTAGGTTGACCGTCTGAATCCTTC
ACTATGGCCTTTATACCTGGATGCAGATCCAACAGGACTTGTCTACATCTACCACAAG
GTGACAAGATCCCGCGATTCTCGTTACCAATAGCAACGATACAAGTCAGATTCCCTG
CGGCGGCGGCAGCGGCGGTTCCCAGTACTACGAGTTCTGCGCAGGGGCCACCCGTAA
AATGATACACATTTACCCCAGTAAAAATCCGCCCATCGGATGACAAGGCTGCACTAG
CCACGGAATAGTCGTCAGATATAGGTATGGAATTGATTGTGGCGGTAGCCCTCTCTAT
CAGCGTGGACTCCTCCTGAGAGAGGGGTTTTGCCATGGTGGCGGCTTAAGGGTTCGA
TCCTCTAGAGTCCGGAGGCTGGATCGGTCCCGGTGTCTTCTATGGAGGTCAAAACAG
CGTGGATGGCGTCTCCAGGCGATCTGACGGTTCACTAAACGAGCTCTGCTTATATAGA
CCTCCCACCGTACACGCCTACCGCCCATTTGCGTCAATGGGGCGGAGTTGTTACGACA
TTTTGGAAAGTCCCGTTGATTTTGGTGCCAAAACAAACTCCCATTGACGTCAATGGGG
TGGAGACTTGGAAATCCCCGTGAGTCAAACCGCTATCCACGCCCATTGATGTACTGCC
AAAACCGCATCACCATGGTAATAGCGATGACTAATACGTAGATGTACTGCCAAGTAG
GAAAGTCCCATAAGGTCATGTACTGGGCATAATGCCAGGCGGGCCATTTACCGTCAT
TGACGTCAATAGGGGGCGTACTTGGCATATGATACACTTGATGTACTGCCAAGTGGG
CAGTTTACCGTAAATACTCCACCCATTGACGTCAATGGAAAGTCCCTATTGGCGTTAC
TATGGGAACATACGTCATTATTGACGTCAATGGGCGGGGGTCGTTGGGCGGTCAGCC
AGGCGGGCCATTTACCGTAAGTTATGTAACGCGGAACTCCATATATGGGCTATGAAC
TAATGACCCCGTAATTGATTACTATTAATAACTAGTCAATAATCAATGTCATTGGGAA
AAGCGCTCCCCTACCCATAACTTCGTATAATGTATGCTATACGAAGTTATTTTGCAGT
TTTAAAATTATGTTTTAAAATGGACTATCATATGCTTACCGTAACTTGAAAGTATTTC
GATTTCTTGGCTTTATATATCTTGTGGAAAGGACGAAACACCGGGCACTCTTCCGTGA
TCTGGTGGATAAATTCGCAAGGGTATCATGGCGGACGACCGGGATTCGAACCCCGGA
TCCGGCCGTCCGCCGTGATCCATGCGGTTACCGCCCGCGTGTCGAACCCAGGTGTGCG
ACGTCAGACAACGGGGGAGCGCTCCTTTTTGGGCCCAT
SEQ ID NO: 29 (STXC0126)
TGGTATGGCTTTTTCCCCGTATCCCCCCAGGTGTCTGCAGGCTCAAAGAG
CAGCGAGAAGCGTTCAGAGGAAAGCGATCCCGTGCCACCTTCCCCGTGCCCGGGCTG
TCCCCGCACGCTGCCGGCTCGGGGATGCGGGGGGAGCGCCGGACCGGAGCGGAGCC
CCGGGCGGCTCGCTGCTGCCCCCTAGCGGGGGAGGGACGTAATTACATCCCTGGGGG
CTTTGGGGGGGGGCTGTCCCTGATATCTATAACAAGAAAATATATATATAATAAGTT
ATCACGTAAGTAGAACATGAAATAACAATATAATTATCGTATGAGTTAAATCTTAAA
AGTCACGTAAAAGATAATCATGCGTCATTTTGACTCACGCGGTCGTTATAGTTCAAAA
TCAGTGACACTTACCGCATTGACAAGCACGCCTCACGGGAGCTCCAAGCGGCGACTG
AGATGTCCTAAATGCACAGCGACGGATTCGCGCTATTTAGAAAGAGAGAGCAATATT
TCAAGAATGCATGCGTCAATTTTACGCAGACTATCTTTCTAGGGTTAATCTAGCTGCA
TCAGGATCATATCGTCGGGTCTTTTTTCCGGCTCAGTCATCGCCCAAGCTGGCGCTAT
CTGGGCATCGGGGAGGAAGAAGCCCGTGCCTTTTCCCGCGAGGTTGAAGCGGCATGG
AAAGAGTTTGCCGAGGATGACTGCTGCTGCATTGACGTTGAGCGAAAACGCACGTTT
ACCATGATGATTCGGGAAGGTGTGGCCATGCACGCCTTTAACGGTGAACTGTTCGTTC
AGGCCACCTGGGATACCAGTTCGTCGCGGCTTTTCCGGACACAGTTCCGGATGGTCA
GCCCGAAGCGCATCAGCAACCCGAACAATACCGGCGACAGCCGGAACTGCCGTGCC
GGTGTGCAGATTAATGACAGCGGTGCGGCGCTGGGATATTACGTCAGCGAGGACGGG
TATCCTGGCTGGATGCCGCAGAAATGGACATGGATACCCCGTGAGTTACCCGGCGGG
CGCGCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGAT
AACTACGATACGGGAGGGCTTACCATCTGGCCCCAGTGCTGCAATGATACCGCGAGA
CCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCA
ACGATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCC
AGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTT
ATCAGGGTTATTGTCTCATGAGCGGATACATATTTGAATGTATTTAGAAAAATAAACA
AATAGGGGTTCCGCGCACATTTCCCCGAAAAGTGCCACCTAAATTGTAAGCGTTAAT
ATTTTGTTAAAATTCGCGTTAAATTTTTGTTAAATCAGCTCATTTTTTAACCAATAGGC
CGAAATCGGCAAAATCCCTTATAAATCAAAAGAATAGACCGAGATAGGGTTGAGTGT
TGTTCCAGTTTGGAACAAGAGTCCACTATTAAAGAACGTGGACTCCAACGTCAAAGG
GCGAAAAACCGTCTATCAGGGCGATGGCCCACTACGTGAACCATCACCCTAATCAAG
TTTTTTGGGGTCGAGGTGCCGTAAAGCACTAAATCGGAACCCTAAAGGGAGCCCCCG
ATTTAGAGCTTGACGGGGAAAGCCGGCGAACGTGGCGAGAAAGGAAGGGAAGAAAG
CGAAAGGAGCGGGCGCTAGGGCGCTGGCAAGTGTAGCGGTCACGCTGCGCGTAACC
ACCACACCCGCCGCGCTTAATGCGCCGCTACAGGGCGCGTCCCATTCGCCATTCAGG
CTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAG
TCACGACGTTGTAAAACGACGGCCAGTGAGCGCGCCTCGTTCATTCACGTTTTTGAAC
CCGTGGAGGACGGGCAGACTCGCGGTGCAAATGTGTTTTACAGCGTGATGGAGCAGA
TGAAGATGCTCGACACGCTGCAGAACACGCAGCTAGATTAACCCTAGAAAGATAATC
ATATTGTGACGTACGTTAAAGATAATCATGTGTAAAATTGACGCATGTGTTTTATCGG
TCTGTATATCGAGGTTTATTTATTAATTTGAATAGATATTAAGTTTTATTATATTTACA
CTTACATACTAATAATAAATTCAACAAACAATTTATTTATGTTTATTTATTTATTAAAA
AAAACAAAAACTCAAAATTTCTTCTATAAAGTAACAAAACTTTTATGAGGGACAGCC
CCCCCCCAAAGCCCCCAGGGATGTAATTACGTCCCTCCCCCGCTAGGGGGCAGCAGC
GAGCCGCCCGGGGCTCCGCTCCGGTCCGGCGCTCCCCCCGCATCCCCGAGCCGGCAG
CGTGCGGGGACAGCCCGGGCACGGGGAAGGTGGCACGGGATCGCTTTCCTCTGAACG
CTTCTCGCTGCTCTTTGAGCCTGCAGACACCTGGGGGGATACGGGGAAAAGGCCTCC
ACGGCCACTAGTCCATAGAGCCCACCGCATCCCCAGCATGCCTGCTATTGTCTTCCCA
ATCCTCCCCCTTGCTGTCCTGCCCCACCCCACCCCCTAGAATAGAATGACACCTACTC
AGACAATGCGATGCAATTTCCTCATTTTATTAGGAAAGGACAGTGGGAGTGGCACCT
TCCAGGGTCAAGGAAGGCACGGGGGAGGGGCAAACAACAGATGGCTGGCAACTAGA
AGGCACAGCTACATGGGGGTAGAGTCATAATCGTGCATCAGGATAGGGCGGTGGTGC
TGCAGCAGCGCGCGAATAAACTGCTGCCGCCGCCGCTCCGTCCTGCAGGAATACAAC
ATGGCAGTGGTCTCCTCAGCGATGATTCGCACCGCCCGCAGCATGAGACGCCTTGTCC
TCCGGGCACAGCAGCGCACCCTGATCTCACTTAAATCAGCACAGTAACTGCAGCACA
GCACCACAATATTGTTCAAAATCCCACAGTGCAAGGCGCTGTATCCAAAGCTCATGG
CGGGGACCACAGAACCCACGTGGCCATCATACCACAAGCGCAGGTAGATTAAGTGGC
GACCCCTCATAAACACGCTGGACATAAACATTACCTCTTTTGGCATGTTGTAATTCAC
CACCTCCCGGTACCATATAAACCTCTGATTAAACATGGCGCCATCCACCACCATCCTA
AACCAGCTGGCCAAAACCTGCCCGCCGGCTATGCACTGCAGGGAACCGGGACTGGAA
CAATGACAGTGGAGAGCCCAGGACTCGTAACCATGGATCATCATGCTCGTCATGATA
TCAATGTTGGCACAACACAGGCACACGTGCATACACTTCCTCAGGATTACAAGCTCCT
CCCGCGTCAGAACCATATCCCAGGGAACAACCCATTCCTGAATCAGCGTAAATCCCA
CACTGCAGGGAAGACCTCGCACGTAACTCACGTTGTGCATTGTCAAAGTGTTACATTC
GGGCAGCAGCGGATGATCCTCCAGTATGGTAGCGCGGGTCTCTGTCTCAAAAGGAGG
TAGGCGATCCCTACTGTACGGAGTGCGCCGAGACAACCGAGATCGTGTTGGTCGTAG
TGTCATGCCAAATGGAACGCCGGACGTAGTCATGGTTGTGGCCATATTATCATCGTGT
TTTTCAAAGGAAAACCACGTCCCCGTGGTTCGGGGGGCCTAGACGTTTTTTTAACCTC
GACTAAACACATGTAAAGCATGTGCACCGAGGCCCCAGATCAGATCCCATACAATGG
GGTACCTTCTGGGCATCCTTCAGCCCCTTGTTGAATACGCTTGAGGAGAGCCATTTGA
CTCTTTCCACAACTATCCAACTCACAACGTGGCACTGGGGTTGTGCCGCCTTTGCAGG
TGTATCTTATACACGTGGCTTTTGGCCGCAGAGGCACCTGTCGCCAGGTGGGGGGTTC
CGCTGCCTGCAAAGGGTCGCTACAGACGTTGTTTGTCTTCAAGAAGCTTCCAGAGGA
ACTGCTTCCTTCACGACATTCAACAGACCTTGCATTCCTTTGGCGAGAGGGGAAAGAC
CCCTAGGAATGCTCGTCAAGAAGACAGGGCCAGGTTTCCGGGCCCTCACATTGCCAA
AAGACGGCAATATGGTGGAAAATAACATATAGACAAACGCACACCGGCCTTATTCCA
AGCGGCTTCGGCCAGTAACGTTAGGGGGGGGGGAGGGAGAGGGGCTTAAAAATCAA
AGGGGTTCTGCCGCGCATCACTATGCGCCACTGGCAGGGACACGTTGCGATACTGGT
GTTTAGTGCTCCACTTAAACTCAGGCACAACCATCCGCGGCAGCTCGGTGAAGTTTTC
ACTCCACAGGCTGCGCACCATCACCAACGCGTTTAGCAGGTCGGGCGCCGATATCTT
GAAGTCGCAGTTGGGGCCTCCGCCCTGCGCGCGCGAGTTGCGATACACAGGGTTGCA
GCACTGGAACACTATCAGCGCCGGGTGGTGCACGCTGGCCAGCACGCTCTTGTCGGA
GATCAGATCCGCGTCCAGGTCCTCCGCGTTGCTCAGGGCGAACGGAGTCAACTTTGG
TAGCTGCCTTCCCAAAAAGGGTGCATGCCCAGGCTTTGAGTTGCACTCGCACCGTAGT
GGCATCAGAAGGTGACCGTGCCCGGTCTGGGCGTTAGGATACAGCGCCTGCATGAAA
GCCTTGATCTGCTTAAAAGCCACCTGAGCCTTTGCGCCTTCAGAGAAGAACATGCCGC
AAGACTTGCCGGAAAACTGATTGGCCGGACAGGCCGCGTCATGCACGCAGCACCTTG
CGTCGGTGTTGGAGATCTGCACCACATTTCGGCCCCACCGGTTCTTCACGATCTTGGC
CTTGCTAGACTGCTCCTTCAGCGCGCGCTGCCCGTTTTCGCTCGTCACATCCATTTCAA
TCACGTGCTCCTTATTTATCATAATGCTCCCGTGTAGACACTTAAGCTCGCCTTCGATC
TCAGCGCAGCGGTGCAGCCACAACGCGCAGCCCGTGGGCTCGTGGTGCTTGTAGGTT
ACCTCTGCAAACGACTGCAGGTACGCCTGCAGGAATCGCCCCATCATCGTCACAAAG
GTCTTGTTGCTGGTGAAGGTCAGCTGCAACCCGCGGTGCTCCTCGTTTAGCCAGGTCT
TGCATACGGCCGCCAGAGCTTCCACTTGGTCAGGCAGTAGCTTGAAGTTTGCCTTTAG
ATCGTTATCCACGTGGTACTTGTCCATCAACGCGCGCGCAGCCTCCATGCCCTTCTCC
CACGCAGACACGATCGGCAGGCTCAGCGGGTTTATCACCGTGCTTTCACTTTCCGCTT
CACTGGACTCTTCCTTTTCCTCTTGCGTCCGCATACCCCGCGCCACTGGGTCGTCTTCA
TTCAGCCGCCGCACCGTGCGCTTACCTCCCTTGCCGTGCTTGATTAGCACCGGTGGGT
TGCTGAAACCCACCATTTGTAGCGCCACATCTTCTCTTTCTTCCTCGCTGTCCACGATC
ACCTCTGGGGATGGCGGGCGCTCGGGCTTGGGAGAGGGGCGCTTCTTTTTCTTTTTGG
ACGCAATGGCCAAATCCGCCGTCGAGGTCGATGGCCGCGGGCTGGGTGTGCGCGGCA
CCAGCGCATCTTGTGACGAGTCTTCTTCGTCCTCGGACTCGAGACGCCGCCTCAGCCG
CTTTTTTGGGGGCGCGCGCTTGTCGTCATCGTCTTTGTAGTCGGGAGGCGGCGGCGAC
GGCGACGGGGACGACACGTCCTCCATGGTTGGTGGACGTCGCGCCGCACCGCGTCCG
CGCTCGGGGGTGGTTTCGCGCTGCTCCTCTTCCCGACTGGCCATGGTGGCCGAGGATA
ACTTCGTATATGGTTTCTTATACGAAGTTATGATCCAGACATGATAAGATACATTGAT
GAGTTTGGACAAACCACAACTAGAATGCAGTGAAAAAAATGCTTTATTTGTGAAATT
TGTGATGCTATTGCTTTATTTGTAACCATTATAAGCTGCAATAAACAAGTTAACAACA
ACAATTGCATTCATTTTATGTTTCAGGTTCAGGGGGAGGTGTGGGAGGTTTTTTAAAG
CAAGTAAAACCTCTACAAATGTGGTATGGCTGATTATGATCCTCTAGAGTCGCAGATC
TGCTACGTATCAAGCTGTGGCAGGGAAACCCTCTGCCTCCCCCGTGATGTAATACTTT
TGCAAGGAATGCGATGAAGTAGAGCCCGCAGTGGCCAAGTGGCTTTGGTCCGTCTCC
TCCACGGATGCCCCTCCACGGCTAGTGGGCGCATGTAGGCGGTGGGCGTCCGCCGCC
TCCAGCAGCAGGTCATAGAGGGGCACCACGTTCTTGCACTTCATGCTGTACAGATGCT
CCATGCCTTTGTTACTCATGTGTCGGATGTGGGAGAGGATGAGGAGGAGCTGGGCCA
GCCGCTGGTGCTGCTGCTGCAGGGTCAGGCCTGCCTTGGCCATCAGGTGGATCAAAG
TGTCTGTGATCTTGTCCAGGACTCGGTGGATATGGTCCTTCTCTTCCAGAGACTTCAG
GGTGCTGGACAGAAATGTGTACACTCCAGAATTAAGCAAAATAATAGATTTGAGGCA
CACAAACTCCTCTCCCTGCAGATTCATCATGCGGAACCGAGATGATGTAGCCAGCAG
CATGTCGAAGATCTCCACCATGCCCTCTACACATTTTCCCTGGTTCCTGTCCAAGAGC
AAGTTAGGAGCAAACAGTAGCTTCACTGGGTGCTCCATGGAGCGCCAGACGAGACCA
ATCATCAGGATCTCTAGCCAGGCACATTCTAGAAGGTGGACCTGATCATGGAGGGTC
AAATCCACAAAGCCTGGCACCCTCTTCGCCCAGTTGATCATGTGAACCAGCTCCCTGT
CTGCCAGGTTGGTCAGTAAGCCCATCATCGAAGCTTCACTGAAGGGTCTGGTAGGAT
CATACTCGGAATAGAGTATGGGGGGCTCAGCATCCAACAAGGCACTGACCATCTGGT
CGGCCGTCAGGGACAAGGCCAGGCTGTTCTTCTTAGAGCGTTTGATCATGAGCGGGC
TTGGCCAAAGGTTGGCAGCTCTCATGTCTCCAGCAGATGGCTCGAGATCGCCATCTTC
CAGCAGGCGCACCATTGCCCCTGTTTCACTATCCAGGTTACGGATATAGTTCATGACA
ATATTTACATTGGTCCAGCCACCAGCTTGCATGATCTCCGGTATTGAAACTCCAGCGC
GGGCCATATCTCGCGCGGCTCCGACACGGGCACTGTGTCCAGACCAGGCCAGGTATC
TCTGACCAGAGTCATCCTAAAATACACAAACAATTAGAATCAGTAGTTTAACACATT
ATACACTTAAAAATTTTATATTTACCTTAGCGCCGTAAATCAATCGATGAGTTGCTTC
AAAAATCCCTTCCAGGGCGCGAGTTGATAGCTGGCTGGTGGCAGATGGCGCGGCAAC
ACCATTTTTTCTGACCCGGCAAAACAGGTAGTTATTCGGATCATCAGCTACACCAGAG
ACGGAAATCCATCGCTCGACCAGTTTAGTGACTCCCAGGCTAAGTGCCTTCTCTACAC
CTGCGGTGCTAACCAGCGTTTTCGTTCTGCCAATATGGATTAACATTCTCCCACCGTC
AGTACGTGAGATATCTTTAACCCTGATCCTGGCAATTTCGGCTATACGTAACAGGGTG
TTATAAGCAATCCCCAGAAATGCCAGATTACGTATATCCTGGCAGCGATCGCTATTTT
CCATGAGTGAACGGACTTGGTCGAAATCAGTGCGTTCGAACGCTAGAGCCTGTTTTG
CACGTTCACCGGCATCAACGTTTTCTTTTCGGATCCGCCGCATAACCAGTGAAACAGC
ATTGCTGTCACTTGGTCGTGGCAGCCCGGACCGACGATGAAGCATGTTTAGCTGGCCC
AAATGTTGCTGGATAGTTTTTACTGCCAGACCGCGCGCTTGAAGATATAGAAGATAA
TCGCGAACATCTTCAGGTTCTGCGGGAAACCATTTCCGGTTATTCAACTTGCACCATG
CCGCCCACGACCGGCAAACGGACAGAAGCATTTTCCAGGTATGCTCAGAAAACGCCT
GGCGATCCCTGAACATGTCCATCAGGTTCTTGCGAACCTCATCACTCGTTGCATCGAC
CGGTAATGCAGGCAAATTTTGGTGTACGGTCAGTAAATTGGACATGGTGGCTACGTA
ATAACTTCGTATATGGTTTCTTATACGAAGTTATGCGGCCGCTTTACGAGGGTAGGAA
GTGGTACGGAAAGTTGGTATAAGACAAAAGTGTTGTGGAATTGCTCCAGGCGATCTG
ACGGTTCACTAAACGAGCTCTGCTTTTATAGGCGCCCACCGTACACGCCTAAAGCTTA
TACGTTCTCTATCACTGATAGGGAGTAAACTGGATATACGTTCTCTATCACTGATAGG
GAGTAAACTGTAGATACGTTCTCTATCACTGATAGGGAGTAAACTGGTCATACGTTCT
CTATCACTGATAGGGAGTAAACTCCTTATACGTTCTCTATCACTGATAGGGAGTAAAG
TCTGCATACGTTCTCTATCACTGATAGGGAGTAAACTCTTCATACGTTCTCTATCACTG
ATAGGGAGTAAACTCGCGGCCGCAGAGAAATGTTCTGGCACCTGCACTTGCACTGGG
GACAGCCTATTTTGCTAGTTTGTTTTGTTTCGTTTTGTTTTGATGGAGAGCGTATGTTA
GTACTATCGATTCACACAAAAAACCAACACACAGATGTAATGAAAATAAAGATATTT
TATTGGATCTGCGATCGCTCCGGTGCCCGTCAGTGGGCAGAGCGCACATCGCCCACA
GTCCCCGAGAAGTTGGGGGGAGGGGTCGGCAATTGAACGGGTGCCTAGAGAAGGTG
GCGCGGGGTAAACTGGGAAAGTGATGTCGTGTACTGGCTCCGCCTTTTTCCCGAGGG
TGGGGGAGAACCGTATGTAAGTGCAGTAGTCGCCGTGAACGTTCTTTTTCGCAACGG
GTTTGCCGCCAGAACACAGCTGAAGCTTCGAGGGGCTCGCATCTCTCCTTCACGCGCC
CGCCGCCCTACCTGAGGCCGCCATCCACGCCGGTTGAGTCGCGTTCTGCCGCCTCCCG
CCTGTGGTGCCTCCTGAACTGCGTCCGCCGTCTAGGTAAGTTTAAAGCTCAGGTCGAG
ACCGGGCCTTTGTCCGGCGCTCCCTTGGAGCCTACCTAGACTCAGCCGGCTCTCCACG
CTTTGCCTGACCCTGCTTGCTCAACTCTACGTCTTTGTTTCGTTTTCTGTTCTGCGCCGT
TACAGATCCAAGCTGTGACCGGCGCCTACGCTAGCGGATCCGCCGCCACCATGTCTA
GACTGGACAAGAGCAAAGTCATAAACTCTGCTCTGGAATTACTCAATGGAGTCGGTA
TCGAAGGCCTGACGACAAGGAAACTCGCTCAAAAGCTGGGAGTTGAGCAGCCTACCC
TGTACTGGCACGTGAAGAACAAGCGGGCCCTGCTCGATGCCCTGCCAATCGAGATGC
TGGACAGGCATCATACCCACTCCTGCCCCCTGGAAGGCGAGTCATGGCAAGACTTTC
TGCGGAACAACGCCAAGTCATACCGCTGTGCTCTTCTCTCACATCGCGACGGGGCTA
AAGTGCATCTCGGCACCCGCCCAACAGAGAAACAGTACGAAACCCTGGAAAATCAG
CTCGCGTTCCTGTGTCAGCAAGGCTTCTCCCTGGAGAACGCACTGTACGCTCTGTCCG
CCGTGGGCCACTTTACACTGGGCTGCGTATTGGAGGAACAGGAGCATCAAGTAGCAA
AAGAGGAAAGAGAGACACCTACCACCGATTCTATGCCCCCACTTCTGAAACAAGCAA
TTGAGCTGTTCGACCGGCAGGGAGCCGAACCTGCCTTCCTTTTCGGCCTGGAACTAAT
CATATGTGGCCTGGAGAAACAGCTAAAGTGCGAAAGCGGCGGGCCGACCGACGCCC
TTGACGATTTTGACTTAGACATGCTCCCAGCCGATGCCCTTGACGACTTTGACCTTGA
TATGCTGCCTGCTGACGCTCTTGACGATTTTGACCTTGACATGCTCCCCGGGTGAACC
GGTCGCTGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGC
GGTGGGCTCTATGGCTTCTGAGGCGGAAAGAACCAGCTGGGGCTCGACTAGAGCTTG
CGGAACCCTTAGAGGGCCTATTTCCCATGATTCCTTCATATTTGCATATACGATACAA
GGCTGTTAGAGAGATAATTAGAATTAATTTGACTGTAAACACAAAGATATTAGTACA
AAATAATAACTTCGTATAATGTATGCTATACGAAGTTATCAGACATGATAAGATACA
TTGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGGTAC
CTCAAGCGCCGGGTTTTCGCGTCATGCACCACGTCCGTGGTCTAGAACTAGTATTATG
CCCAGTACATGACCTTATGGGACTTTCCTACTTGGCAGTACATCTACGTATTAGTCAT
CGCTATTACCATGGTGATGCGGTTTTGGCAGTACATCAATGGGCGTGGATAGCGGTTT
GACTCACGGGGATTTCCAAGTCTCCACCCCATTGACGTCAATGGGAGTTTGTTTTGGC
ACCAAAATCAACGGGACTTTCCAAAATGTCGTAACAACTCCGCCCCATTGACGCAAA
TGGGCGGTAGGCGTGTACGGTGGGAGGTTTATATAAGCAGAGCTCGTTTAGTGAACC
GTCAGATCGCCTGGAGACGCCATCCACGCTGTTTTGACCTCCATAGAAGAGAATTCG
CCGCCACCATGAAAACATTTAACATTTCTCAACAGGATCTAGAATTAGTAGAAGTAG
CGACAGAGAAGATTACAATGCTTTATGAGGATAATAAACATCATGTGGGAGCGGCAA
TTCGTACGAAAACAGGAGAAATCATTTCGGCAGTACATATTGAAGCGTATATAGGAC
GAGTAACTGTTTGTGCAGAAGCCATTGCGATTGGTAGTGCAGTTTCGAATGGACAAA
AGGATTTTGACACGATTGTAGCTGTTAGACACCCTTATTCTGACGAAGTAGATAGAA
GTATTCGAGTGGTAAGTCCTTGTGGTATGTGTAGGGAGTTGATTTCAGACTATGCACC
AGATTGTTTTGTGTTAATAGAAATGAATGGCAAGTTAGTCAAAACTACGATTGAAGA
ACTCATTCCACTCAAATATACCCGAAATTAAACCGGTCGCTGATCAGCCTCGACTGTG
CCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTG
AGTAGGTGTCATTCTATTCTGGGGGGTGGGGTGGGGCAGGACAGCAAGGGGGAGGA
TTGGGAAGACAATAGCAGGCATGCTGGGGATGCGGTGGGCTCTATGGCTTCTGAGGC
GGAAAGAACCAGCTGGGGCTCGACTAGAGCTTGCGGAACCCTTAGGGCCCATTGGTA
TGGCTTGGGAAAAGCGCTCCCCTACCCATAACTTCGTATAATGTATGCTATACGAAGT
TATTTTGCAGTTTTAAAATTATGTTTTAAAATGGACTATCATATGCTTACCGTAACTTG
AAAGTATTTCGATTTCTTGGCTTTATATATCTTGTGGAAAGGACGAAACACCGGGCAC
TCTTCCGTGATCTGGTGGATAAATTCGCAAGGGTATCATGGCGGACGACCGGGATTC
GAACCCCGGATCCGGCCGTCCGCCGTGATCCATGCGGTTACCGCCCGCGTGTCGAAC
CCAGGTGTGCGACGTCAGACAACGGGGGAGCGCTCCTTTTTGGGCCCAT
SEQ ID NO: 30 (STXC0123)
TGGTATGGCTTTTTCCCCGTATCCCCCCAGGTGTCTGCAGGCTCAAAGAG
CAGCGAGAAGCGTTCAGAGGAAAGCGATCCCGTGCCACCTTCCCCGTGCCCGGGCTG
TCCCCGCACGCTGCCGGCTCGGGGATGCGGGGGGAGCGCCGGACCGGAGCGGAGCC
CCGGGCGGCTCGCTGCTGCCCCCTAGCGGGGGAGGGACGTAATTACATCCCTGGGGG
CTTTGGGGGGGGGCTGTCCCTGATATCTATAACAAGAAAATATATATATAATAAGTT
ATCACGTAAGTAGAACATGAAATAACAATATAATTATCGTATGAGTTAAATCTTAAA
AGTCACGTAAAAGATAATCATGCGTCATTTTGACTCACGCGGTCGTTATAGTTCAAAA
TCAGTGACACTTACCGCATTGACAAGCACGCCTCACGGGAGCTCCAAGCGGCGACTG
AGATGTCCTAAATGCACAGCGACGGATTCGCGCTATTTAGAAAGAGAGAGCAATATT
TCAAGAATGCATGCGTCAATTTTACGCAGACTATCTTTCTAGGGTTAATCTAGCTGCA
TCAGGATCATATCGTCGGGTCTTTTTTCCGGCTCAGTCATCGCCCAAGCTGGCGCTAT
CTGGGCATCGGGGAGGAAGAAGCCCGTGCCTTTTCCCGCGAGGTTGAAGCGGCATGG
AAAGAGTTTGCCGAGGATGACTGCTGCTGCATTGACGTTGAGCGAAAACGCACGTTT
ACCATGATGATTCGGGAAGGTGTGGCCATGCACGCCTTTAACGGTGAACTGTTCGTTC
AGGCCACCTGGGATACCAGTTCGTCGCGGCTTTTCCGGACACAGTTCCGGATGGTCA
GCCCGAAGCGCATCAGCAACCCGAACAATACCGGCGACAGCCGGAACTGCCGTGCC
GGTGTGCAGATTAATGACAGCGGTGCGGCGCTGGGATATTACGTCAGCGAGGACGGG
TATCCTGGCTGGATGCCGCAGAAATGGACATGGATACCCCGTGAGTTACCCGGCGGG
CGCGCTTGGCGTAATCATGGTCATAGCTGTTTCCTGTGTGAAATTGTTATCCGCTCAC
AATTCCACACAACATACGAGCCGGAAGCATAAAGTGTAAAGCCTGGGGTGCCTAATG
AGTGAGCTAACTCACATTAATTGCGTTGCGCTCACTGCCCGCTTTCCAGTCGGGAAAC
CTGTCGTGCCAGCTGCATTAATGAATCGGCCAACGCGCGGGGAGAGGCGGTTTGCGT
ATTGGGCGCTCTTCCGCTTCCTCGCTCACTGACTCGCTGCGCTCGGTCGTTCGGCTGC
GGCGAGCGGTATCAGCTCACTCAAAGGCGGTAATACGGTTATCCACAGAATCAGGGG
ATAACGCAGGAAAGAACATGTGAGCAAAAGGCCAGCAAAAGGCCAGGAACCGTAAA
AAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCG
TTTCCCCCTGGAAGCTCCCTCGTGCGCTCTCCTGTTCCGACCCTGCCGCTTACCGGATA
CCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCG
TTCAGCCCGACCGCTGCGCCTTATCCGGTAACTATCGTCTTGAGTCCAACCCGGTAAG
ACACGACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTAGAAG
GACAGTATTTGGTATCTGCGCTCTGCTGAAGCCAGTTACCTTCGGAAAAAGAGTTGGT
AGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGG
GGTCTGACGCTCAGTGGAACGAAAACTCACGTTAAGGGATTTTGGTCATGAGATTAT
CAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCT
ATCTCAGCGATCTGTCTATTTCGTTCATCCATAGTTGCCTGACTCCCCGTCGTGTAGAT
AACTACGATACGGGAGGGCTTACCATCTGGCCCCAGTGCTGCAATGATACCGCGAGA
CCCACGCTCACCGGCTCCAGATTTATCAGCAATAAACCAGCCAGCCGGAAGGGCCGA
GCGCAGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATTGCTA
CAGGCATCGTGGTGTCACGCTCGTCGTTTGGTATGGCTTCATTCAGCTCCGGTTCCCA
ACGATCAAGGCGAGTTACATGATCCCCCATGTTGTGCAAAAAAGCGGTTAGCTCCTT
CGGTCCTCCGATCGTTGTCAGAAGTAAGTTGGCCGCAGTGTTATCACTCATGGTTATG
GCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTG
CCCGGCGTCAATACGGGATAATACCGCGCCACATAGCAGAACTTTAAAAGTGCTCAT
CATTGGAAAACGTTCTTCGGGGCGAAAACTCTCAAGGATCTTACCGCTGTTGAGATCC
AGTTCGATGTAACCCACTCGTGCACCCAACTGATCTTCAGCATCTTTTACTTTCACCA
GCGTTTCTGGGTGAGCAAAAACAGGAAGGCAAAATGCCGCAAAAAAGGGAATAAGG
GCGACACGGAAATGTTGAATACTCATACTCTTCCTTTTTCAATATTATTGAAGCATTT
ATCAGGGTTATTGTCTCATGAGCGGATACATATTTGAATGTATTTAGAAAAATAAACA
AATAGGGGTTCCGCGCACATTTCCCCGAAAAGTGCCACCTAAATTGTAAGCGTTAAT
ATTTTGTTAAAATTCGCGTTAAATTTTTGTTAAATCAGCTCATTTTTTAACCAATAGGC
CGAAATCGGCAAAATCCCTTATAAATCAAAAGAATAGACCGAGATAGGGTTGAGTGT
TGTTCCAGTTTGGAACAAGAGTCCACTATTAAAGAACGTGGACTCCAACGTCAAAGG
GCGAAAAACCGTCTATCAGGGCGATGGCCCACTACGTGAACCATCACCCTAATCAAG
TTTTTTGGGGTCGAGGTGCCGTAAAGCACTAAATCGGAACCCTAAAGGGAGCCCCCG
ATTTAGAGCTTGACGGGGAAAGCCGGCGAACGTGGCGAGAAAGGAAGGGAAGAAAG
CGAAAGGAGCGGGCGCTAGGGCGCTGGCAAGTGTAGCGGTCACGCTGCGCGTAACC
ACCACACCCGCCGCGCTTAATGCGCCGCTACAGGGCGCGTCCCATTCGCCATTCAGG
CTGCGCAACTGTTGGGAAGGGCGATCGGTGCGGGCCTCTTCGCTATTACGCCAGCTG
GCGAAAGGGGGATGTGCTGCAAGGCGATTAAGTTGGGTAACGCCAGGGTTTTCCCAG
TCACGACGTTGTAAAACGACGGCCAGTGAGCGCGCCTCGTTCATTCACGTTTTTGAAC
CCGTGGAGGACGGGCAGACTCGCGGTGCAAATGTGTTTTACAGCGTGATGGAGCAGA
TGAAGATGCTCGACACGCTGCAGAACACGCAGCTAGATTAACCCTAGAAAGATAATC
ATATTGTGACGTACGTTAAAGATAATCATGTGTAAAATTGACGCATGTGTTTTATCGG
TCTGTATATCGAGGTTTATTTATTAATTTGAATAGATATTAAGTTTTATTATATTTACA
CTTACATACTAATAATAAATTCAACAAACAATTTATTTATGTTTATTTATTTATTAAAA
AAAACAAAAACTCAAAATTTCTTCTATAAAGTAACAAAACTTTTATGAGGGACAGCC
CCCCCCCAAAGCCCCCAGGGATGTAATTACGTCCCTCCCCCGCTAGGGGGCAGCAGC
GAGCCGCCCGGGGCTCCGCTCCGGTCCGGCGCTCCCCCCGCATCCCCGAGCCGGCAG
CGTGCGGGGACAGCCCGGGCACGGGGAAGGTGGCACGGGATCGCTTTCCTCTGAACG
CTTCTCGCTGCTCTTTGAGCCTGCAGACACCTGGGGGGATACGGGGAAAAGGCCTCC
ACGGCCACTAGTCCATAGAGCCCACCGCATCCCCAGCATGCCTGCTATTGTCTTCCCA
ATCCTCCCCCTTGCTGTCCTGCCCCACCCCACCCCCTAGAATAGAATGACACCTACTC
AGACAATGCGATGCAATTTCCTCATTTTATTAGGAAAGGACAGTGGGAGTGGCACCT
TCCAGGGTCAAGGAAGGCACGGGGGAGGGGCAAACAACAGATGGCTGGCAACTAGA
AGGCACAGCTACATGGGGGTAGAGTCATAATCGTGCATCAGGATAGGGCGGTGGTGC
TGCAGCAGCGCGCGAATAAACTGCTGCCGCCGCCGCTCCGTCCTGCAGGAATACAAC
ATGGCAGTGGTCTCCTCAGCGATGATTCGCACCGCCCGCAGCATGAGACGCCTTGTCC
TCCGGGCACAGCAGCGCACCCTGATCTCACTTAAATCAGCACAGTAACTGCAGCACA
GCACCACAATATTGTTCAAAATCCCACAGTGCAAGGCGCTGTATCCAAAGCTCATGG
CGGGGACCACAGAACCCACGTGGCCATCATACCACAAGCGCAGGTAGATTAAGTGGC
GACCCCTCATAAACACGCTGGACATAAACATTACCTCTTTTGGCATGTTGTAATTCAC
CACCTCCCGGTACCATATAAACCTCTGATTAAACATGGCGCCATCCACCACCATCCTA
AACCAGCTGGCCAAAACCTGCCCGCCGGCTATGCACTGCAGGGAACCGGGACTGGAA
CAATGACAGTGGAGAGCCCAGGACTCGTAACCATGGATCATCATGCTCGTCATGATA
TCAATGTTGGCACAACACAGGCACACGTGCATACACTTCCTCAGGATTACAAGCTCCT
CCCGCGTCAGAACCATATCCCAGGGAACAACCCATTCCTGAATCAGCGTAAATCCCA
CACTGCAGGGAAGACCTCGCACGTAACTCACGTTGTGCATTGTCAAAGTGTTACATTC
GGGCAGCAGCGGATGATCCTCCAGTATGGTAGCGCGGGTCTCTGTCTCAAAAGGAGG
TAGGCGATCCCTACTGTACGGAGTGCGCCGAGACAACCGAGATCGTGTTGGTCGTAG
TGTCATGCCAAATGGAACGCCGGACGTAGTCATGGTTGTGGCCATATTATCATCGTGT
TTTTCAAAGGAAAACCACGTCCCCGTGGTTCGGGGGGCCTAGACGTTTTTTTAACCTC
GACTAAACACATGTAAAGCATGTGCACCGAGGCCCCAGATCAGATCCCATACAATGG
GGTACCTTCTGGGCATCCTTCAGCCCCTTGTTGAATACGCTTGAGGAGAGCCATTTGA
CTCTTTCCACAACTATCCAACTCACAACGTGGCACTGGGGTTGTGCCGCCTTTGCAGG
TGTATCTTATACACGTGGCTTTTGGCCGCAGAGGCACCTGTCGCCAGGTGGGGGGTTC
CGCTGCCTGCAAAGGGTCGCTACAGACGTTGTTTGTCTTCAAGAAGCTTCCAGAGGA
ACTGCTTCCTTCACGACATTCAACAGACCTTGCATTCCTTTGGCGAGAGGGGAAAGAC
CCCTAGGAATGCTCGTCAAGAAGACAGGGCCAGGTTTCCGGGCCCTCACATTGCCAA
AAGACGGCAATATGGTGGAAAATAACATATAGACAAACGCACACCGGCCTTATTCCA
AGCGGCTTCGGCCAGTAACGTTAGGGGGGGGGGAGGGAGAGGGGCTTAAAAATCAA
AGGGGTTCTGCCGCGCATCACTATGCGCCACTGGCAGGGACACGTTGCGATACTGGT
GTTTAGTGCTCCACTTAAACTCAGGCACAACCATCCGCGGCAGCTCGGTGAAGTTTTC
ACTCCACAGGCTGCGCACCATCACCAACGCGTTTAGCAGGTCGGGCGCCGATATCTT
GAAGTCGCAGTTGGGGCCTCCGCCCTGCGCGCGCGAGTTGCGATACACAGGGTTGCA
GCACTGGAACACTATCAGCGCCGGGTGGTGCACGCTGGCCAGCACGCTCTTGTCGGA
GATCAGATCCGCGTCCAGGTCCTCCGCGTTGCTCAGGGCGAACGGAGTCAACTTTGG
TAGCTGCCTTCCCAAAAAGGGTGCATGCCCAGGCTTTGAGTTGCACTCGCACCGTAGT
GGCATCAGAAGGTGACCGTGCCCGGTCTGGGCGTTAGGATACAGCGCCTGCATGAAA
GCCTTGATCTGCTTAAAAGCCACCTGAGCCTTTGCGCCTTCAGAGAAGAACATGCCGC
AAGACTTGCCGGAAAACTGATTGGCCGGACAGGCCGCGTCATGCACGCAGCACCTTG
CGTCGGTGTTGGAGATCTGCACCACATTTCGGCCCCACCGGTTCTTCACGATCTTGGC
CTTGCTAGACTGCTCCTTCAGCGCGCGCTGCCCGTTTTCGCTCGTCACATCCATTTCAA
TCACGTGCTCCTTATTTATCATAATGCTCCCGTGTAGACACTTAAGCTCGCCTTCGATC
TCAGCGCAGCGGTGCAGCCACAACGCGCAGCCCGTGGGCTCGTGGTGCTTGTAGGTT
ACCTCTGCAAACGACTGCAGGTACGCCTGCAGGAATCGCCCCATCATCGTCACAAAG
GTCTTGTTGCTGGTGAAGGTCAGCTGCAACCCGCGGTGCTCCTCGTTTAGCCAGGTCT
TGCATACGGCCGCCAGAGCTTCCACTTGGTCAGGCAGTAGCTTGAAGTTTGCCTTTAG
ATCGTTATCCACGTGGTACTTGTCCATCAACGCGCGCGCAGCCTCCATGCCCTTCTCC
CACGCAGACACGATCGGCAGGCTCAGCGGGTTTATCACCGTGCTTTCACTTTCCGCTT
CACTGGACTCTTCCTTTTCCTCTTGCGTCCGCATACCCCGCGCCACTGGGTCGTCTTCA
TTCAGCCGCCGCACCGTGCGCTTACCTCCCTTGCCGTGCTTGATTAGCACCGGTGGGT
TGCTGAAACCCACCATTTGTAGCGCCACATCTTCTCTTTCTTCCTCGCTGTCCACGATC
ACCTCTGGGGATGGCGGGCGCTCGGGCTTGGGAGAGGGGCGCTTCTTTTTCTTTTTGG
ACGCAATGGCCAAATCCGCCGTCGAGGTCGATGGCCGCGGGCTGGGTGTGCGCGGCA
CCAGCGCATCTTGTGACGAGTCTTCTTCGTCCTCGGACTCGAGACGCCGCCTCAGCCG
CTTTTTTGGGGGCGCGCGCTTGTCGTCATCGTCTTTGTAGTCGGGAGGCGGCGGCGAC
GGCGACGGGGACGACACGTCCTCCATGGTTGGTGGACGTCGCGCCGCACCGCGTCCG
CGCTCGGGGGTGGTTTCGCGCTGCTCCTCTTCCCGACTGGCCATGGTGGCCGAGGATA
ACTTCGTATATGGTTTCTTATACGAAGTTATGATCCAGACATGATAAGATACATTGAT
GAGTTTGGACAAACCACAACTAGAATGCAGTGAAAAAAATGCTTTATTTGTGAAATT
TGTGATGCTATTGCTTTATTTGTAACCATTATAAGCTGCAATAAACAAGTTAACAACA
ACAATTGCATTCATTTTATGTTTCAGGTTCAGGGGGAGGTGTGGGAGGTTTTTTAAAG
CAAGTAAAACCTCTACAAATGTGGTATGGCTGATTATGATCCTCTAGAGTCGCAGATC
TGCTACGTATCAAGCTGTGGCAGGGAAACCCTCTGCCTCCCCCGTGATGTAATACTTT
TGCAAGGAATGCGATGAAGTAGAGCCCGCAGTGGCCAAGTGGCTTTGGTCCGTCTCC
TCCACGGATGCCCCTCCACGGCTAGTGGGCGCATGTAGGCGGTGGGCGTCCGCCGCC
TCCAGCAGCAGGTCATAGAGGGGCACCACGTTCTTGCACTTCATGCTGTACAGATGCT
CCATGCCTTTGTTACTCATGTGTCGGATGTGGGAGAGGATGAGGAGGAGCTGGGCCA
GCCGCTGGTGCTGCTGCTGCAGGGTCAGGCCTGCCTTGGCCATCAGGTGGATCAAAG
TGTCTGTGATCTTGTCCAGGACTCGGTGGATATGGTCCTTCTCTTCCAGAGACTTCAG
GGTGCTGGACAGAAATGTGTACACTCCAGAATTAAGCAAAATAATAGATTTGAGGCA
CACAAACTCCTCTCCCTGCAGATTCATCATGCGGAACCGAGATGATGTAGCCAGCAG
CATGTCGAAGATCTCCACCATGCCCTCTACACATTTTCCCTGGTTCCTGTCCAAGAGC
AAGTTAGGAGCAAACAGTAGCTTCACTGGGTGCTCCATGGAGCGCCAGACGAGACCA
ATCATCAGGATCTCTAGCCAGGCACATTCTAGAAGGTGGACCTGATCATGGAGGGTC
AAATCCACAAAGCCTGGCACCCTCTTCGCCCAGTTGATCATGTGAACCAGCTCCCTGT
CTGCCAGGTTGGTCAGTAAGCCCATCATCGAAGCTTCACTGAAGGGTCTGGTAGGAT
CATACTCGGAATAGAGTATGGGGGGCTCAGCATCCAACAAGGCACTGACCATCTGGT
CGGCCGTCAGGGACAAGGCCAGGCTGTTCTTCTTAGAGCGTTTGATCATGAGCGGGC
TTGGCCAAAGGTTGGCAGCTCTCATGTCTCCAGCAGATGGCTCGAGATCGCCATCTTC
CAGCAGGCGCACCATTGCCCCTGTTTCACTATCCAGGTTACGGATATAGTTCATGACA
ATATTTACATTGGTCCAGCCACCAGCTTGCATGATCTCCGGTATTGAAACTCCAGCGC
GGGCCATATCTCGCGCGGCTCCGACACGGGCACTGTGTCCAGACCAGGCCAGGTATC
TCTGACCAGAGTCATCCTAAAATACACAAACAATTAGAATCAGTAGTTTAACACATT
ATACACTTAAAAATTTTATATTTACCTTAGCGCCGTAAATCAATCGATGAGTTGCTTC
AAAAATCCCTTCCAGGGCGCGAGTTGATAGCTGGCTGGTGGCAGATGGCGCGGCAAC
ACCATTTTTTCTGACCCGGCAAAACAGGTAGTTATTCGGATCATCAGCTACACCAGAG
ACGGAAATCCATCGCTCGACCAGTTTAGTGACTCCCAGGCTAAGTGCCTTCTCTACAC
CTGCGGTGCTAACCAGCGTTTTCGTTCTGCCAATATGGATTAACATTCTCCCACCGTC
AGTACGTGAGATATCTTTAACCCTGATCCTGGCAATTTCGGCTATACGTAACAGGGTG
TTATAAGCAATCCCCAGAAATGCCAGATTACGTATATCCTGGCAGCGATCGCTATTTT
CCATGAGTGAACGGACTTGGTCGAAATCAGTGCGTTCGAACGCTAGAGCCTGTTTTG
CACGTTCACCGGCATCAACGTTTTCTTTTCGGATCCGCCGCATAACCAGTGAAACAGC
ATTGCTGTCACTTGGTCGTGGCAGCCCGGACCGACGATGAAGCATGTTTAGCTGGCCC
AAATGTTGCTGGATAGTTTTTACTGCCAGACCGCGCGCTTGAAGATATAGAAGATAA
TCGCGAACATCTTCAGGTTCTGCGGGAAACCATTTCCGGTTATTCAACTTGCACCATG
CCGCCCACGACCGGCAAACGGACAGAAGCATTTTCCAGGTATGCTCAGAAAACGCCT
GGCGATCCCTGAACATGTCCATCAGGTTCTTGCGAACCTCATCACTCGTTGCATCGAC
CGGTAATGCAGGCAAATTTTGGTGTACGGTCAGTAAATTGGACATGGTGGCTACGTA
ATAACTTCGTATATGGTTTCTTATACGAAGTTATGCGGCCGCTTTACGAGGGTAGGAA
GTGGTACGGAAAGTTGGTATAAGACAAAAGTGTTGTGGAATTGCTCCAGGCGATCTG
ACGGTTCACTAAACGAGCTCTGCTTTTATAGGCGCCCACCGTACACGCCTAAAGCTTA
TACGTTCTCTATCACTGATAGGGAGTAAACTGGATATACGTTCTCTATCACTGATAGG
GAGTAAACTGTAGATACGTTCTCTATCACTGATAGGGAGTAAACTGGTCATACGTTCT
CTATCACTGATAGGGAGTAAACTCCTTATACGTTCTCTATCACTGATAGGGAGTAAAG
TCTGCATACGTTCTCTATCACTGATAGGGAGTAAACTCTTCATACGTTCTCTATCACTG
ATAGGGAGTAAACTCGCGGCCGCAGAGAAATGTTCTGGCACCTGCACTTGCACTGGG
GACAGCCTATTTTGCTAGTTTGTTTTGTTTCGTTTTGTTTTGATGGAGAGCGTATGTTA
GTACTATCGATTCACACAAAAAACCAACACACAGATGTAATGAAAATAAAGATATTT
TATTGGATCTGCGATCGCTCCGGTGCCCGTCAGTGGGCAGAGCGCACATCGCCCACA
GTCCCCGAGAAGTTGGGGGGAGGGGTCGGCAATTGAACGGGTGCCTAGAGAAGGTG
GCGCGGGGTAAACTGGGAAAGTGATGTCGTGTACTGGCTCCGCCTTTTTCCCGAGGG
TGGGGGAGAACCGTATGTAAGTGCAGTAGTCGCCGTGAACGTTCTTTTTCGCAACGG
GTTTGCCGCCAGAACACAGCTGAAGCTTCGAGGGGCTCGCATCTCTCCTTCACGCGCC
CGCCGCCCTACCTGAGGCCGCCATCCACGCCGGTTGAGTCGCGTTCTGCCGCCTCCCG
CCTGTGGTGCCTCCTGAACTGCGTCCGCCGTCTAGGTAAGTTTAAAGCTCAGGTCGAG
ACCGGGCCTTTGTCCGGCGCTCCCTTGGAGCCTACCTAGACTCAGCCGGCTCTCCACG
CTTTGCCTGACCCTGCTTGCTCAACTCTACGTCTTTGTTTCGTTTTCTGTTCTGCGCCGT
TACAGATCCAAGCTGTGACCGGCGCCTACGCTAGCGGATCCGCCGCCACCATGTCTA
GACTGGACAAGAGCAAAGTCATAAACTCTGCTCTGGAATTACTCAATGGAGTCGGTA
TCGAAGGCCTGACGACAAGGAAACTCGCTCAAAAGCTGGGAGTTGAGCAGCCTACCC
TGTACTGGCACGTGAAGAACAAGCGGGCCCTGCTCGATGCCCTGCCAATCGAGATGC
TGGACAGGCATCATACCCACTCCTGCCCCCTGGAAGGCGAGTCATGGCAAGACTTTC
TGCGGAACAACGCCAAGTCATACCGCTGTGCTCTTCTCTCACATCGCGACGGGGCTA
AAGTGCATCTCGGCACCCGCCCAACAGAGAAACAGTACGAAACCCTGGAAAATCAG
CTCGCGTTCCTGTGTCAGCAAGGCTTCTCCCTGGAGAACGCACTGTACGCTCTGTCCG
CCGTGGGCCACTTTACACTGGGCTGCGTATTGGAGGAACAGGAGCATCAAGTAGCAA
AAGAGGAAAGAGAGACACCTACCACCGATTCTATGCCCCCACTTCTGAAACAAGCAA
TTGAGCTGTTCGACCGGCAGGGAGCCGAACCTGCCTTCCTTTTCGGCCTGGAACTAAT
CATATGTGGCCTGGAGAAACAGCTAAAGTGCGAAAGCGGCGGGCCGACCGACGCCC
TTGACGATTTTGACTTAGACATGCTCCCAGCCGATGCCCTTGACGACTTTGACCTTGA
TATGCTGCCTGCTGACGCTCTTGACGATTTTGACCTTGACATGCTCCCCGGGTGAACC
GGTCGCTGATCAGCCTCGACTGTGCCTTCTAGTTGCCAGCCATCTGTTGTTTGCCCCTC
CCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTG
GGGCAGGACAGCAAGGGGGAGGATTGGGAAGACAATAGCAGGCATGCTGGGGATGC
GGTGGGCTCTATGGCTTCTGAGGCGGAAAGAACCAGCTGGGGCTCGACTAGAGCTTG
CGGAACCCTTAGAGGGCCTATTTCCCATGATTCCTTCATATTTGCATATACGATACAA
GGCTGTTAGAGAGATAATTAGAATTAATTTGACTGTAAACACAAAGATATTAGTACA
AAATAATAACTTCGTATAATGTATGCTATACGAAGTTATCAGACATGATAAGATACA
TTGATGAGTTTGGACAAACCACAACTAGAATGCAGTGAAAAAAATGCTTTATTTGTG
AAATTTGTGATGCTATTGCTTTATTTGTAACCATTATAAGCTGCAATAAACAAGGTAC
CTCAAGCGCCGGGTTTTCGCGTCATGCACCACGTCCGTGGTTCAAGCGCCGGGTTTTC
GCGTCATGCACCACGTCCGTGGGCCCTCGGGTACTTCAACGTCAGCAGTAACTGTAA
ATCCGAGCCGTTCATAGAAGGGCAAATTCCTTGGCGCTGACGTTTCAAGAAAGGCTG
GCACTCCGGCTCGTTCTGCGGCTTCTACTCCGGGCAATACCACCGCGGAACCAAGGC
CCTTTCCCTGATGATCGGGGCTAACGCCCACAGTAGCGAGGAACCAAGCTGGTTCTTT
AGGGCGGTGAGGGGCGAGGAGTCCTTCCATTTGTTGCTGAGCCGCGAGACGAGAGCC
ACTAAGCTCAGCCATTCGGGGACCAATTTCTGCAAATACAGCCCCGGCCTCAACGCT
CTCCGGAGTCGTCCACACTGCCACTGCAGCCCCGTCGTCGGCGACCCAAACTTTACCG
ATGTCCAATCCTACCCTGGTCAAAAAAAGTTCTTGCAATTCTGTAACCCGTTCAATAT
GTCTATCAGGATCAACTGTGTGGCGTGTAGCGGGATAATCCGCGAAAGCGGCAGCCA
ATGTTCTCACGGCCCTAGGGACGTCGTCTCGAGTTGCCAGTCTGACAGTAGGTTTATA
TTCTGTCATGGTGGCGGCGAATTCTCTTCTATGGAGGTCAAAACAGCGTGGATGGCGT
CTCCAGGCGATCTGACGGTTCACTAAACGAGCTCTGCTTATATAAACCTCCCACCGTA
CACGCCTACCGCCCATTTGCGTCAATGGGGCGGAGTTGTTACGACATTTTGGAAAGTC
CCGTTGATTTTGGTGCCAAAACAAACTCCCATTGACGTCAATGGGGTGGAGACTTGG
AAATCCCCGTGAGTCAAACCGCTATCCACGCCCATTGATGTACTGCCAAAACCGCAT
CACCATGGTAATAGCGATGACTAATACGTAGATGTACTGCCAAGTAGGAAAGTCCCA
TAAGGTCATGTACTGGGCATAATACTAGTTCTTGGGAAAAGCGCTCCCCTACCCATAA
CTTCGTATAATGTATGCTATACGAAGTTATTTTGCAGTTTTAAAATTATGTTTTAAAAT
GGACTATCATATGCTTACCGTAACTTGAAAGTATTTCGATTTCTTGGCTTTATATATCT
TGTGGAAAGGACGAAACACCGGGCACTCTTCCGTGATCTGGTGGATAAATTCGCAAG
GGTATCATGGCGGACGACCGGGATTCGAACCCCGGATCCGGCCGTCCGCCGTGATCC
ATGCGGTTACCGCCCGCGTGTCGAACCCAGGTGTGCGACGTCAGACAACGGGGGAGC
GCTCCTTTTTGGGCCCAT
SEQ ID NO: 31 (STXC0133)
actcttcctttttcaatattattgaagcatttatcagggttattgtctcatgagcggatacatatttgaatgtatttagaaaaataaa
caaataggggttccgcgcacatttccccgaaaagtgccacctaaattgtaagcgttaatattttgttaaaattcgcgttaaatttttgttaaatcagct
cattttttaaccaataggccgaaatcggcaaaatcccttataaatcaaaagaatagaccgagatagggttgagtgttgttccagtttggaacaag
agtccactattaaagaacgtggactccaacgtcaaagggcgaaaaaccgtctatcagggcgatggcccactacgtgaaccatcaccctaatc
aagttttttggggtcgaggtgccgtaaagcactaaatcggaaccctaaagggagcccccgatttagagcttgacggggaaagccggcgaac
gtggcgagaaaggaagggaagaaagcgaaaggagcgggcgctagggcgctggcaagtgtagcggtcacgctgcgcgtaaccaccaca
cccgccgcgcttaatgcgccgctacagggcgcgtcccattcgccattcaggctgcgcaactgttgggaagggcgatcggtgcgggcctctt
cgctattacgccagctggcgaaagggggatgtgctgcaaggcgattaagttgggtaacgccagggttttcccagtcacgacgttgtaaaacg
acggccagtgagcgcgcctcgttcattcacgtttttgaacccgtggaggacgggcagactcgcggtgcaaatgtgttttacagcgtgatggag
cagatgaagatgctcgacacgctgcagaacacgcagctagattaaccctagaaagataatcatattgtgacgtacgttaaagataatcatgtgt
aaaattgacgcatgtgttttatcggtctgtatatcgaggtttatttattaatttgaatagatattaagttttattatatttacacttacatactaataataaat
tcaacaaacaatttatttatgtttatttatttattaaaaaaaacaaaaactcaaaatttcttctataaagtaacaaaacttttatgagggacagcccccc
cccaaagcccccagggatgtaattacgtccctcccccgctagggggcagcagcgagccgcccggggctccgctccggtccggcgctccc
cccgcatccccgagccggcagcgtgcggggacagcccgggcacggggaaggtggcacgggatcgctttcctctgaacgcttctcgctgct
ctttgagcctgcagacacctggggggatacggggaaaaggcctccacggccactagtccatagagcccaccgcatccccagcatgcctgc
tattgtcttcccaatcctcccccttgctgtcctgccccaccccaccccctagaatagaatgacacctactcagacaatgcgatgcaatttcctcatt
ttattaggaaaggacagtgggagtggcaccttccagggtcaaggaaggcacgggggaggggcaaacaacagatggctggcaactagaag
gcacagctacatgggggtagagtcataatcgtgcatcaggatagggcggtggtgctgcagcagcgcgcgaataaactgctgccgccgccg
ctccgtcctgcaggaatacaacatggcagtggtctcctcagcgatgattcgcaccgcccgcagcatgagacgccttgtcctccgggcacagc
agcgcaccctgatctcacttaaatcagcacagtaactgcagcacagcaccacaatattgttcaaaatcccacagtgcaaggcgctgtatccaa
agctcatggcggggaccacagaacccacgtggccatcataccacaagcgcaggtagattaagtggcgacccctcataaacacgctggaca
taaacattacctcttttggcatgttgtaattcaccacctcccggtaccatataaacctctgattaaacatggcgccatccaccaccatcctaaacca
gctggccaaaacctgcccgccggctatgcactgcagggaaccgggactggaacaatgacagtggagagcccaggactcgtaaccatgga
tcatcatgctcgtcatgatatcaatgttggcacaacacaggcacacgtgcatacacttcctcaggattacaagctcctcccgcgtcagaaccata
tcccagggaacaacccattcctgaatcagcgtaaatcccacactgcagggaagacctcgcacgtaactcacgttgtgcattgtcaaagtgtta
cattcgggcagcagcggatgatcctccagtatggtagcgcgggtctctgtctcaaaaggaggtaggcgatccctactgtacggagtgcgccg
agacaaccgagatcgtgttggtcgtagtgtcatgccaaatggaacgccggacgtagtcatggttgtggccatattatcatcgtgtttttcaaagg
aaaaccacgtccccgtggttcggggggcctagacgtttttttaacctcgactaaacacatgtaaagcatgtgcaccgaggccccagatcagat
cccatacaatggggtaccttctgggcatccttcagccccttgttgaatacgcttgaggagagccatttgactctttccacaactatccaactcaca
acgtggcactggggttgtgccgcctttgcaggtgtatcttatacacgtggcttttggccgcagaggcacctgtcgccaggtggggggttccgc
tgcctgcaaagggtcgctacagacgttgtttgtcttcaagaagcttccagaggaactgcttccttcacgacattcaacagaccttgcattcctttg
gcgagaggggaaagacccctaggaatgctcgtcaagaagacagggccaggtttccgggccctcacattgccaaaagacggcaatatggtg
gaaaataacatatagacaaacgcacaccggccttattccaagcggcttcggccagtaacgttagggggggggggggagaggggcttaaa
aatcaaaggggttctgccgcgcatcactatgcgccactggcagggacacgttgcgatactggtgtttagtgctccacttaaactcaggcacaa
ccatccgcggcagctcggtgaagttttcactccacaggctgcgcaccatcaccaacgcgtttagcaggtcgggcgccgatatcttgaagtcg
cagttggggcctccgccctgcgcgcgcgagttgcgatacacagggttgcagcactggaacactatcagcgccgggtggtgcacgctggcc
agcacgctcttgtcggagatcagatccgcgtccaggtcctccgcgttgctcagggcgaacggagtcaactttggtagctgccttcccaaaaag
ggtgcatgcccaggctttgagttgcactcgcaccgtagtggcatcagaaggtgaccgtgcccggtctgggcgttaggatacagcgcctgcat
gaaagccttgatctgcttaaaagccacctgagcctttgcgccttcagagaagaacatgccgcaagacttgccggaaaactgattggccggac
aggccgcgtcatgcacgcagcaccttgcgtcggtgttggagatctgcaccacatttcggccccaccggttcttcacgatcttggccttgctaga
ctgctccttcagcgcgcgctgcccgttttcgctcgtcacatccatttcaatcacgtgctccttatttatcataatgctcccgtgtagacacttaagct
cgccttcgatctcagcgcagcggtgcagccacaacgcgcagcccgtgggctcgtggtgcttgtaggttacctctgcaaacgactgcaggtac
gcctgcaggaatcgccccatcatcgtcacaaaggtcttgttgctggtgaaggtcagctgcaacccgcggtgctcctcgtttagccaggtcttgc
atacggccgccagagcttccacttggtcaggcagtagcttgaagtttgcctttagatcgttatccacgtggtacttgtccatcaacgcgcgcgca
gcctccatgcccttctcccacgcagacacgatcggcaggctcagcgggtttatcaccgtgctttcactttccgcttcactggactcttccttttcct
cttgcgtccgcataccccgcgccactgggtcgtcttcattcagccgccgcaccgtgcgcttacctcccttgccgtgcttgattagcaccggtgg
gttgctgaaacccaccatttgtagcgccacatcttctctttcttcctcgctgtccacgatcacctctggggatggcgggcgctcgggcttgggag
aggggcgcttctttttctttttggacgcaatggccaaatccgccgtcgaggtcgatggccgcgggctgggtgtgcgcggcaccagcgcatctt
gtgacgagtcttcttcgtcctcggactcgagacgccgcctcagccgcttttttgggggcgcgcgcttgtcgtcatcgtctttgtagtcgggaggc
ggcggcgacggcgacggggacgacacgtcctccatggttggtggacgtcgcgccgcaccgcgtccgcgctcgggggtggtttcgcgctg
ctcctcttcccgactggccatggtggccgaggataacttcgtatatggtttcttatacgaagttatgatccagacatgataagatacattgatgagt
ttggacaaaccacaactagaatgcagtgaaaaaaatgctttatttgtgaaatttgtgatgctattgctttatttgtaaccattataagctgcaataaac
aagttaacaacaacaattgcattcattttatgtttcaggttcagggggaggtgtgggaggttttttaaagcaagtaaaacctctacaaatgtggtat
ggctgattatgatcctctagagtcgcagatctgctacgtatcaagctgtggcagggaaaccctctgcctcccccgtgatgtaatacttttgcaag
gaatgcgatgaagtagagcccgcagtggccaagtggctttggtccgtctcctccacggatgcccctccacggctagtgggcgcatgtaggc
ggtgggcgtccgccgcctccagcagcaggtcatagaggggcaccacgttcttgcacttcatgctgtacagatgctccatgcctttgttactcat
gtgtcggatgtgggagaggatgaggaggagctgggccagccgctggtgctgctgctgcagggtcaggcctgccttggccatcaggtggat
caaagtgtctgtgatcttgtccaggactcggtggatatggtccttctcttccagagacttcagggtgctggacagaaatgtgtacactccagaatt
aagcaaaataatagatttgaggcacacaaactcctctccctgcagattcatcatgcggaaccgagatgatgtagccagcagcatgtcgaagat
ctccaccatgccctctacacattttccctggttcctgtccaagagcaagttaggagcaaacagtagcttcactgggtgctccatggagcgccag
acgagaccaatcatcaggatctctagccaggcacattctagaaggtggacctgatcatggagggtcaaatccacaaagcctggcaccctctt
cgcccagttgatcatgtgaaccagctccctgtctgccaggttggtcagtaagcccatcatcgaagcttcactgaagggtctggtaggatcatac
tcggaatagagtatggggggctcagcatccaacaaggcactgaccatctggtcggccgtcagggacaaggccaggctgttcttcttagagc
gtttgatcatgagcgggcttggccaaaggttggcagctctcatgtctccagcagatggctcgagatcgccatcttccagcaggcgcaccattg
cccctgtttcactatccaggttacggatatagttcatgacaatatttacattggtccagccaccagcttgcatgatctccggtattgaaactccagc
gcgggccatatctcgcgcggctccgacacgggcactgtgtccagaccaggccaggtatctctgaccagagtcatcctaaaatacacaaaca
attagaatcagtagtttaacacattatacacttaaaaattttatatttaccttagcgccgtaaatcaatcgatgagttgcttcaaaaatcccttccagg
gcgcgagttgatagctggctggtggcagatggcgcggcaacaccattttttctgacccggcaaaacaggtagttattcggatcatcagctaca
ccagagacggaaatccatcgctcgaccagtttagtgactcccaggctaagtgccttctctacacctgcggtgctaaccagcgttttcgttctgcc
aatatggattaacattctcccaccgtcagtacgtgagatatctttaaccctgatcctggcaatttcggctatacgtaacagggtgttataagcaatc
cccagaaatgccagattacgtatatcctggcagcgatcgctattttccatgagtgaacggacttggtcgaaatcagtgcgttcgaacgctagag
cctgttttgcacgttcaccggcatcaacgttttcttttcggatccgccgcataaccagtgaaacagcattgctgtcacttggtcgtggcagcccgg
accgacgatgaagcatgtttagctggcccaaatgttgctggatagtttttactgccagaccgcgcgcttgaagatatagaagataatcgcgaac
atcttcaggttctgcgggaaaccatttccggttattcaacttgcaccatgccgcccacgaccggcaaacggacagaagcattttccaggtatgc
tcagaaaacgcctggcgatccctgaacatgtccatcaggttcttgcgaacctcatcactcgttgcatcgaccggtaatgcaggcaaattttggtg
tacggtcagtaaattggacatggtggctacgtaataacttcgtatatggtttcttatacgaagttatgcggccgctttacgagggtaggaagtggt
acggaaagttggtataagacaaaagtgttgtggaattgctccaggcgatctgacggttcactaaacgagctctgcttttataggcgcccaccgt
acacgcctaaagcttatacgttctctatcactgatagggagtaaactggatatacgttctctatcactgatagggagtaaactgtagatacgttctc
tatcactgatagggagtaaactggtcatacgttctctatcactgatagggagtaaactccttatacgttctctatcactgatagggagtaaagtctg
catacgttctctatcactgatagggagtaaactcttcatacgttctctatcactgatagggagtaaactcgcggccgcagagaaatgttctggca
cctgcacttgcactggggacagcctattttgctagtttgttttgtttcgttttgttttgatggagagcgtatgttagtactatcgattcacacaaaaaac
caacacacagatgtaatgaaaataaagatattttattggatctgcgatcgctccggtgcccgtcagtgggcagagcgcacatcgcccacagtc
cccgagaagttggggggaggggtcggcaattgaacgggtgcctagagaaggtggcgcggggtaaactgggaaagtgatgtcgtgtactg
gctccgcctttttcccgaggggggggagaaccgtatgtaagtgcagtagtcgccgtgaacgttctttttcgcaacgggtttgccgccagaaca
cagctgaagcttcgaggggctcgcatctctccttcacgcgcccgccgccctacctgaggccgccatccacgccggttgagtcgcgttctgcc
gcctcccgcctgtggtgcctcctgaactgcgtccgccgtctaggtaagtttaaagctcaggtcgagaccgggcctttgtccggcgctcccttg
gagcctacctagactcagccggctctccacgctttgcctgaccctgcttgctcaactctacgtctttgtttcgttttctgttctgcgccgttacagat
ccaagctgtgaccggcgcctacgctagcggatccgccgccaccatgtctagactggacaagagcaaagtcataaactctgctctggaattac
tcaatggagtcggtatcgaaggcctgacgacaaggaaactcgctcaaaagctgggagttgagcagcctaccctgtactggcacgtgaagaa
caagcgggccctgctcgatgccctgccaatcgagatgctggacaggcatcatacccactcctgccccctggaaggcgagtcatggcaaga
ctttctgcggaacaacgccaagtcataccgctgtgctcttctctcacatcgcgacggggctaaagtgcatctcggcacccgcccaacagaga
aacagtacgaaaccctggaaaatcagctcgcgttcctgtgtcagcaaggcttctccctggagaacgcactgtacgctctgtccgccgtgggc
cactttacactgggctgcgtattggaggaacaggagcatcaagtagcaaaagaggaaagagagacacctaccaccgattctatgcccccact
tctgaaacaagcaattgagctgttcgaccggcagggagccgaacctgccttccttttcggcctggaactaatcatatgtggcctggagaaaca
gctaaagtgcgaaagcggcgggccgaccgacgcccttgacgattttgacttagacatgctcccagccgatgcccttgacgactttgaccttga
tatgctgcctgctgacgctcttgacgattttgaccttgacatgctccccgggtgaaccggtcgctgatcagcctcgactgtgccttctagttgcca
gccatctgttgtttgcccctcccccgtgccttccttgaccctggaaggtgccactcccactgtcctttcctaataaaatgaggaaattgcatcgcat
tgtctgagtaggtgtcattctattctggggggtggggggggcaggacagcaagggggaggattgggaagacaatagcaggcatgctggg
gatgcggtgggctctatggcttctgaggcggaaagaaccagctggggctcgactagagcttgcggaacccttagagggcctatttcccatgg
tggtagaactagtattatgcccagtacatgaccttatgggactttcctacttggcagtacatctacgtattagtcatcgctattaccatggtgatgcg
gttttggcagtacatcaatgggcgtggatagcggtttgactcacggggatttccaagtctccaccccattgacgtcaatgggagtttgttttggca
ccaaaatcaacgggactttccaaaatgtcgtaacaactccgccccattgacgcaaatgggcggtaggcgtgtacggtgggaggtttatataag
cagagctcgtttagtgaaccgtcagatcgcctggagacgccatccacgctgttttgacctccatagaagagaattcgccgccaccatgacaga
atataaacctactgtcagactggcaactcgagacgacgtccctagggccgtgagaacattggctgccgctttcgcggattatcccgctacacg
ccacacagttgatcctgatagacatattgaacgggttacagaattgcaagaactttttttgaccagggtaggattggacatcggtaaagtttgggt
cgccgacgacggggctgcagtggcagtgtggacgactccggagagcgttgaggccggggctgtatttgcagaaattggtccccgaatggc
tgagcttagtggctctcgtctcgcggctcagcaacaaatggaaggactcctcgcccctcaccgccctaaagaaccagcttggttcctcgctact
gtgggcgttagccccgatcatcagggaaagggccttggttccgcggtggtattgcccggagtagaagccgcagaacgagccggagtgcca
gcctttcttgaaacgtcagcgccaaggaatttgcccttctatgaacggctcggatttacagttactgctgacgttgaagtacccgagggcccac
ggacgtggtgcatgacgcgaaaacccggcgcttgagtttaaaccgctgatcagcctcgactgtgccttctagttgccagccatctgttgtttgc
ccccccccgtgccttccttgaccctggaaggtgccactcccactgtcctttcctaataaaatgaggaaattgcatcgcattgtctgagtaggtgt
cattctattctggggggtggggggggcaggacagcaagggggaggattgggaagacaatagcaggcatgctggggatgcggtgggctc
tatggcttctgaggcggaaagaaccagctggggctcgactagagcttgcggaacccttagtttaaacgggcccttaattaatcgatgtaggat
gttgcccctcctgacgcggtaggagaaggggagggtgccctgcatgtctgccgctgctcttgctcttgccgctgctgaggaggggggcgca
tctgccgcagcaccggatgcatctgggaaaagcaaaaaaggggctcgtccctgtttccggaggaatttgcaagcggggtcttgcatgacgg
ggaggcaaacccccgttcgccgcagtccggccggcccgagactcgaaccgggggtcctgcgactcaacccttggaaaataaccctccgg
ctacagggagcgagccacttaatgctttcgctttccagcctaaccgcttacgccgcgcgcggccagtggccaaaaaagctagcgcagcagc
cgccgcgcctggaaggaagccaaaaggagcgctcccccgttgtctgacgtcgcacacctgggttcgacacgcgggcggtaaccgcatgg
atcacggcggacggccggatccggggttcgaaccccggtcgtccgccatgatacccttgcgaatttatccaccagaccacggaagagtgcc
cgcttacaggctctccttttgcacggtctagagcgtcaacgactgcgcacgcctcaccggccagagcgtcccgaccatggagcactttttgcc
gctgcgcaacatctggaaccgcgtccgcgactttccgcgcgcctccaccaccgccgccggcatcacctggatgtccaggtacatctacgga
ttacggggcccattggtatggctttttccccgtatccccccaggtgtctgcaggctcaaagagcagcgagaagcgttcagaggaaagcgatc
ccgtgccaccttccccgtgcccgggctgtccccgcacgctgccggctcggggatgcggggggagcgccggaccggagcggagccccg
ggcggctcgctgctgccccctagcgggggagggacgtaattacatccctgggggctttgggggggggctgtccctgatatctataacaaga
aaatatatatataataagttatcacgtaagtagaacatgaaataacaatataattatcgtatgagttaaatcttaaaagtcacgtaaaagataatcat
gcgtcattttgactcacgcggtcgttatagttcaaaatcagtgacacttaccgcattgacaagcacgcctcacgggagctccaagcggcgact
gagatgtcctaaatgcacagcgacggattcgcgctatttagaaagagagagcaatatttcaagaatgcatgcgtcaattttacgcagactatctt
tctagggttaatctagctgcatcaggatcatatcgtcgggtcttttttccggctcagtcatcgcccaagctggcgctatctgggcatcggggagg
aagaagcccgtgccttttcccgcgaggttgaagcggcatggaaagagtttgccgaggatgactgctgctgcattgacgttgagcgaaaacgc
acgtttaccatgatgattcgggaaggtgtggccatgcacgcctttaacggtgaactgttcgttcaggccacctgggataccagttcgtcgcggc
ttttccggacacagttccggatggtcagcccgaagcgcatcagcaacccgaacaataccggcgacagccggaactgccgtgccggtgtgc
agattaatgacagcggtgcggcgctgggatattacgtcagcgaggacgggtatcctggctggatgccgcagaaatggacatggataccccg
tgagttacccggcgggcgcgcttggcgtaatcatggtcatagctgtttcctgtgtgaaattgttatccgctcacaattccacacaacatacgagc
cggaagcataaagtgtaaagcctggggtgcctaatgagtgagctaactcacattaattgcgttgcgctcactgcccgctttccagtcgggaaa
cctgtcgtgccagctgcattaatgaatcggccaacgcgcggggagaggcggtttgcgtattgggcgctcttccgcttcctcgctcactgactc
gctgcgctcggtcgttcggctgcggcgagcggtatcagctcactcaaaggcggtaatacggttatccacagaatcaggggataacgcagga
aagaacatgtgagcaaaaggccagcaaaaggccaggaaccgtaaaaaggccgcgttgctggcgtttttccataggctccgcccccctgac
gagcatcacaaaaatcgacgctcaagtcagaggtggcgaaacccgacaggactataaagataccaggcgtttccccctggaagctccctcg
tgcgctctcctgttccgaccctgccgcttaccggatacctgtccgcctttctcccttcgggaagcgtggcgctttctcatagctcacgctgtaggt
atctcagttcggtgtaggtcgttcgctccaagctgggctgtgtgcacgaaccccccgttcagcccgaccgctgcgccttatccggtaactatcg
tcttgagtccaacccggtaagacacgacttatcgccactggcagcagccactggtaacaggattagcagagcgaggtatgtaggcggtgcta
cagagttcttgaagtggtggcctaactacggctacactagaaggacagtatttggtatctgcgctctgctgaagccagttaccttcggaaaaag
agttggtagctcttgatccggcaaacaaaccaccgctggtagcggtggtttttttgtttgcaagcagcagattacgcgcagaaaaaaaggatct
caagaagatcctttgatcttttctacggggtctgacgctcagtggaacgaaaactcacgttaagggattttggtcatgagattatcaaaaaggatc
ttcacctagatccttttaaattaaaaatgaagttttaaatcaatctaaagtatatatgagtaaacttggtctgacagttaccaatgcttaatcagtgag
gcacctatctcagcgatctgtctatttcgttcatccatagttgcctgactccccgtcgtgtagataactacgatacgggagggcttaccatctggc
cccagtgctgcaatgataccgcgagacccacgctcaccggctccagatttatcagcaataaaccagccagccggaagggccgagcgcaga
agtggtcctgcaactttatccgcctccatccagtctattaattgttgccgggaagctagagtaagtagttcgccagttaatagtttgcgcaacgttg
ttgccattgctacaggcatcgtggtgtcacgctcgtcgtttggtatggcttcattcagctccggttcccaacgatcaaggcgagttacatgatccc
ccatgttgtgcaaaaaagcggttagctccttcggtcctccgatcgttgtcagaagtaagttggccgcagtgttatcactcatggttatggcagca
ctgcataattctcttactgtcatgccatccgtaagatgcttttctgtgactggtgagtactcaaccaagtcattctgagaatagtgtatgcggcgac
cgagttgctcttgcccggcgtcaatacgggataataccgcgccacatagcagaactttaaaagtgctcatcattggaaaacgttcttcggggcg
aaaactctcaaggatcttaccgctgttgagatccagttcgatgtaacccactcgtgcacccaactgatcttcagcatcttttactttcaccagcgttt
ctgggtgagcaaaaacaggaaggcaaaatgccgcaaaaaagggaataagggcgacacggaaatgttgaatactcat
SEQ ID NO: 32 (STXC0137)
ACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCAC
ATTTCCCCGAAAAGTGCCACCTAAATTGTAAGCGTTAATATTTTGTTAAAATTCGCGT
TAAATTTTTGTTAAATCAGCTCATTTTTTAACCAATAGGCCGAAATCGGCAAAATCCC
TTATAAATCAAAAGAATAGACCGAGATAGGGTTGAGTGTTGTTCCAGTTTGGAACAA
GAGTCCACTATTAAAGAACGTGGACTCCAACGTCAAAGGGCGAAAAACCGTCTATCA
GGGCGATGGCCCACTACGTGAACCATCACCCTAATCAAGTTTTTTGGGGTCGAGGTG
CCGTAAAGCACTAAATCGGAACCCTAAAGGGAGCCCCCGATTTAGAGCTTGACGGGG
AAAGCCGGCGAACGTGGCGAGAAAGGAAGGGAAGAAAGCGAAAGGAGCGGGCGCT
AGGGCGCTGGCAAGTGTAGCGGTCACGCTGCGCGTAACCACCACACCCGCCGCGCTT
AATGCGCCGCTACAGGGCGCGTCCCATTCGCCATTCAGGCTGCGCAACTGTTGGGAA
GGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACG
ACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACG
CTGCAGAACACGCAGCTAGATTAACCCTAGAAAGATAATCATATTGTGACGTACGTT
AAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATA
AATTCAACAAACAATTTATTTATGTTTATTTATTTATTAAAAAAAACAAAAACTCAAA
ATTTCTTCTATAAAGTAACAAAACTTTTATGGAGGGGTGGAGTCGTGACGTGAATTAC
GTCATAGGGTTAGGGAGGTCCTGTATTAGAGGTCACGTGAGTGTTTTGCGACATTTTG
CGACACCATGTGGTCACGCTGGGTATTTAAGCCCGAGTGAGCACGCAGGGTCTCCAT
TTTGAAGCGGGAGGTTTGAACGCGCAGCCGCCATGCCGGGGTTTTACGAGATTGTGA
TTAAGGTCCCCAGCGACCTTGACGAGCATCTGCCCGGCATTTCTGACAGCTTTGTGAA
CTGGGTGGCCGAGAAGGAATGGGAGTTGCCGCCAGATTCTGACATGGATCTGAATCT
GATTGAGCAGGCACCCCTGACCGTGGCCGAGAAGCTGCAGCGCGACTTTCTGACGGA
ATGGCGCCGTGTGAGTAAGGCCCCGGAGGCCCTTTTCTTTGTGCAATTTGAGAAGGG
AGAGAGCTACTTCCACATGCACGTGCTCGTGGAAACCACCGGGGTGAAATCCATGGT
TTTGGGACGTTTCCTGAGTCAGATTCGCGAAAAACTGATTCAGAGAATTTACCGCGG
GATCGAGCCGACTTTGCCAAACTGGTTCGCGGTCACAAAGACCAGAAATGGCGCCGG
AGGCGGGAACAAGGTGGTGGATGAGTGCTACATCCCCAATTACTTGCTCCCCAAAAC
CCAGCCTGAGCTCCAGTGGGCGTGGACTAATATGGAACAGTATTTAAGCGCCTGTTT
GAATCTCACGGAGCGTAAACGGTTGGTGGCGCAGCATCTGACGCACGTGTCGCAGAC
GCAGGAGCAGAACAAAGAGAATCAGAATCCCAATTCTGATGCGCCGGTGATCAGATC
AAAAACTTCAGCCAGGTACATGGAGCTGGTCGGGTGGCTCGTGGACAAGGTGAGTTT
GGGGACCCTTGATTGTTCTTTCTTTTTCGCTATTGTAAAATTCATGTTATATGGAGGGG
GCAAAGTTTTCAGGGTGTTGTTTAGAATGGGAAGATGTCCCTTGTATCACCATGGACC
CTCATGATAATTTTGTTTCTTTCACTTTCTACTCTGTTGACAACCGTTGTCTCCTCTTAT
TTTCTTTTCATTTTCTGTAACTTTTTCGTTAAACTTTAGCTTGCATTTGTAACGAATTTT
TAAATTCACTTTTGTTTATTTGTCAGATTGTAAGTACTTTCTCTAATCACTTTTTTTTCA
AGGCAATCAGGGTATATTATATTGTACTTCAGCACAGTTTTAGAGAACATAACTTCGT
ATAAAGTATACTATACGAAGTTATCGGGCCCCTCTGCTAACCATGTTCATGCCTTCTT
CTTTTTCCTACAGATGTCAGAACTCATTAAAGAGAATATGCACATGAAGCTGTATATG
GAAGGTACTGTAGACAACCACCATTTCAAATGCACGTCCGAAGGTGAGGGGAAGCCA
TACGAGGGTACCCAAACTATGCGCATCAAAGTGGTTGAGGGTGGCCCCCTGCCATTC
GCATTCGACATCCTGGCAACTAGCTTTCTTTACGGTTCCAAGACATTCATAAATCATA
CCCAGGGTATTCCCGATTTCTTCAAACAATCCTTCCCGGAAGGGTTTACTTGGGAGCG
GGTCACGACATATGAAGACGGGGGTGTTCTTACAGCCACACAGGATACGAGTTTGCA
AGACGGTTGTCTTATCTATAACGTGAAGATTCGGGGTGTGAATTTCACATCCAATGGC
CCGGTGATGCAGAAAAAAACACTGGGCTGGGAAGCATTTACGGAGACGTTGTATCCC
GCCGATGGAGGTCTCGAGGGCCGAAACGATATGGCCCTCAAGTTGGTAGGTGGTTCT
CACCTTATAGCAAACATTAAGACCACGTATCGATCAAAAAAACCCGCTAAGAATCTG
AAAATGCCAGGCGTGTATTATGTTGATTACAGACTGGAGCGAATAAAAGAGGCTAAC
AATGAGACCTACGTCGAACAGCATGAAGTCGCTGTAGCTAGATATTGCGACCTCCCG
TCAAAGTTGGGCCATAAATTGAATTAACCTCAGGTGCAGGCTGCCTATCAGAAGGTG
GTGGCTGGTGTGGCCAATGCCCTGGCTCACAAATACCACTGAGATCTTTTTCCCTCTG
CCAAAAATTATGGGGACATCATGAAGCCCCTTGAGCATCTGACTTCTGGCTAATAAA
GGAAATTTATTTTCATTGCAATAGTGTGTTGGAATTTTTTGTGTCTCTCACTCGGAAGG
ACATATGGGAGGGCAAATCATTTAAAACATCAGAATGAGTATTTGGTTTAGAGTTTG
GCAACATATGCCCATATGCTGGCTGCCATGAACAAAGGTTGGCTATAAAGAGGTCAT
CAGTATATGAAACAGCCCCCTGCTGTCCATTCCTTATTCCATAGAAAAGCCTTGACTT
GAGGTTAGATTTTTTTTATATTTTGTTTTGTGTTATTTTTTTCTTTAACATCCCTAAAAT
TTTCCTTACATGTTTTACTAGCCAGATTTTTCCTCCTCTCCTGACTACTCCCAGTCATA
GCTGTCCCTCTTCTCTTATGGAGATCATAACTTCGTATAAAGTATACTATACGAAGTT
ATAATTGTTATAATTAAATGATAAGGTAGAATATTTCTGCATATAAATTCTGGCTGGC
GTGGAAATATTCTTATTGGTAGAAACAACTACACCCTGGTCATCATCCTGCCTTTCTC
TTTATGGTTACAATGATATACACTGTTTGAGATGAGGATAAAATACTCTGAGTCCAAA
CCGGGCCCCTCTGCTAACCATGTTCATGCCTTCTTCTTTTTCCTACAGGGGATTACCTC
GGAGAAGCAGTGGATCCAGGAGGACCAGGCCTCATACATCTCCTTCAATGCGGCCTC
CAACTCGCGGTCCCAAATCAAGGCTGCCTTGGACAATGCGGGAAAGATTATGAGCCT
GACTAAAACCGCCCCCGACTACCTGGTGGGCCAGCAGCCCGTGGAGGACATTTCCAG
CAATCGGATTTATAAAATTTTGGAACTAAACGGGTACGATCCCCAATATGCGGCTTCC
GTCTTTCTGGGATGGGCCACGAAAAAGTTCGGCAAGAGGAACACCATCTGGCTGTTT
GGGCCTGCAACTACCGGGAAGACCAACATCGCGGAGGCCATAGCCCACACTGTGCCC
TTCTACGGGTGCGTAAACTGGACCAATGAGAACTTTCCCTTCAACGACTGTGTCGACA
AGATGGTGATCTGGTGGGAGGAGGGGAAGATGACCGCCAAGGTCGTGGAGTCGGCC
AAAGCCATTCTCGGAGGAAGCAAGGTGCGCGTGGACCAGAAATGCAAGTCCTCGGCC
CAGATAGACCCGACTCCCGTGATCGTCACCTCCAACACCAACATGTGCGCCGTGATT
GACGGGAACTCAACGACCTTCGAACACCAGCAGCCGTTGCAAGACCGGATGTTCAAA
TTTGAACTCACCCGCCGTCTGGATCATGACTTTGGGAAGGTCACCAAGCAGGAAGTC
AAAGACTTTTTCCGGTGGGCAAAGGATCACGTGGTTGAGGTGGAGCATGAATTCTAC
GTCAAAAAGGGTGGAGCCAAGAAAAGACCCGCCCCCAGTGACGCAGATATAAGTGA
GCCCAAACGGGTGCGCGAGTCAGTTGCGCAGCCATCGACGTCAGACGCGGAAGCTTC
GATCAACTACGCAGACAGGTACCAAAACAAATGTTCTCGTCACGTGGGCATGAATCT
GATGCTGTTTCCCTGCAGACAATGCGAGAGAATGAATCAGAATTCAAATATCTGCTTC
ACTCACGGACAGAAAGACTGTTTAGAGTGCTTTCCCGTGTCAGAATCTCAACCCGTTT
CTGTCGTCAAAAAGGCGTATCAGAAACTGTGCTACATTCATCATATCATGGGAAAGG
TGCCAGACGCTTGCACTGCCTGCGATCTGGTCAATGTGGATTTGGATGACTGCATCTT
TGAACAATAAATGATTTGTAAATAAATTTAGTAGTCATGTCTTTTGTTGATCACCCTC
CAGATTGGTTGGAAGAAGTTGGTGAAGGTCTTCGCGAGTTTTTGGGCCTTGAAGCGG
GCCCACCGAAACCAAAACCCAATCAGCAGCATCAAGATCAAGCCCGTGGTCTTGTGC
TGCCTGGTTATAACTATCTCGGACCCGGAAACGGTCTCGATCGAGGAGAGCCTGTCA
ACAGGGCAGACGAGGTCGCGCGAGAGCACGACATCTCGTACAACGAGCAGCTTGAG
GCGGGAGACAACCCCTACCTCAAGTACAACCACGCGGACGCCGAGTTTCAGGAGAA
GCTCGCCGACGACACATCCTTCGGGGGAAACCTCGGAAAGGCAGTCTTTCAGGCCAA
GAAAAGGGTTCTCGAACCTTTTGGCCTGGTTGAAGAGGGTGCTAAGACGGCCCCTAC
CGGAAAGCGGATAGACGACCACTTTCCAAAAAGAAAGAAGGCTCGGACCGAAGAGG
ACTCCAAGCCTTCCACCTCGTCAGACGCCGAAGCTGGACCCAGCGGATCCCAGCAGC
TGCAAATCCCAGCCCAACCAGCCTCAAGTTTGGGAGCTGATACAATGTCTGCGGGAG
GTGGCGGCCCATTGGGCGACAATAACCAAGGTGCCGATGGAGTGGGCAATGCCTCGG
GAGATTGGCATTGCGATTCCACGTGGATGGGGGACAGAGTCGTCACCAAGTCCACCC
GAACCTGGGTGCTGCCCAGCTACAACAACCACCAGTACCGAGAGATCAAAAGCGGCT
CCGTCGACGGAAGCAACGCCAACGCCTACTTTGGATACAGCACCCCCTGGGGGTACT
TTGACTTTAACCGCTTCCACAGCCACTGGAGCCCCCGAGACTGGCAAAGACTCATCA
ACAACTACTGGGGCTTCAGACCCCGGTCCCTCAGAGTCAAAATCTTCAACATTCAAGT
CAAAGAGGTCACGGTGCAGGACTCCACCACCACCATCGCCAACAACCTCACCTCCAC
CGTCCAAGTGTTTACGGACGACGACTACCAGCTGCCCTACGTCGTCGGCAACGGGAC
CGAGGGATGCCTGCCGGCCTTCCCTCCGCAGGTCTTTACGCTGCCGCAGTACGGTTAC
GCGACGCTGAACCGCGACAACACAGAAAATCCCACCGAGAGGAGCAGCTTCTTCTGC
CTAGAGTACTTTCCCAGCAAGATGCTGAGAACGGGCAACAACTTTGAGTTTACCTAC
AACTTTGAGGAGGTGCCCTTCCACTCCAGCTTCGCTCCCAGTCAGAACCTGTTCAAGC
TGGCCAACCCGCTGGTGGACCAGTACTTGTACCGCTTCGTGAGCACAAATAACACTG
GCGGAGTCCAGTTCAACAAGAACCTGGCCGGGAGATACGCCAACACCTACAAAAACT
GGTTCCCGGGGCCCATGGGCCGAACCCAGGGCTGGAACCTGGGCTCCGGGGTCAACC
GCGCCAGTGTCAGCGCCTTCGCCACGACCAATAGGATGGAGCTCGAGGGCGCGAGTT
ACCAGGTGCCCCCGCAGCCGAACGGCATGACCAACAACCTCCAGGGCAGCAACACCT
ATGCCCTGGAGAACACTATGATCTTCAACAGCCAGCCGGCGAACCCGGGCACCACCG
CCACGTACCTCGAGGGCAACATGCTCATCACCAGCGAGAGCGAGACGCAGCCGGTGA
ACCGCGTGGCGTACAACGTCGGCGGGCAGATGGCCACCAACAACCAGAGCTCCACCA
CTGCCCCCGCGACCGGCACGTACAACCTCCAGGAAATCGTGCCCGGCAGCGTGTGGA
TGGAGAGGGACGTGTACCTCCAAGGACCCATCTGGGCCAAGATCCCAGAGACGGGG
GCGCACTTTCACCCCTCTCCGGCCATGGGCGGATTCGGACTCAAACACCCACCGCCCA
TGATGCTCATCAAGAACACGCCTGTGCCCGGAAATATCACCAGCTTCTCGGACGTGC
CCGTCAGCAGCTTCATCACCCAGTACAGCACCGGGCAGGTCACCGTGGAGATGGAGT
GGGAGCTCAAGAAGGAAAACTCCAAGAGGTGGAACCCAGAGATCCAGTACACAAAC
AACTACAACGACCCCCAGTTTGTGGACTTTGCCCCGGACAGCACCGGGGAATACAGA
ACCACCAGACCTATCGGAACCCGATACCTTACCCGACCCCTTTAATTGCTTGTTAATC
AATAAACCGTTTAATTCGTTTCAGTTGAACTTTGGTCTCTGCGTATTTCTTTCTTATCT
AGTTTCCATGGCTACGTAGATAAGTAGCATGGCGGGTTAATCATTAACTACAGCCCG
GGCGTTTAAACAGCGGGCGGAGGGGTGGAGTCGTGACGTGAATTACGTCATAGGGTT
AGGGAGGTCCTGTATTAGAGGTCACGTGAGTGTTTTGCGACATTTTGCGACACCATGT
GGTCCGCGGCCGCAAGGATCTGCGATCGCTCCGGTGCCCGTCAGTGGGCAGAGCGCA
CATCGCCCACAGTCCCCGAGAAGTTGGGGGGAGGGGTCGGCAATTGAACGGGTGCCT
AGAGAAGGTGGCGCGGGGTAAACTGGGAAAGTGATGTCGTGTACTGGCTCCGCCTTT
TTCCCGAGGGTGGGGGAGAACCGTATATAAGTGCAGTAGTCGCCGTGAACGTTCTTT
TTCGCAACGGGTTTGCCGCCAGAACACAGCTGAAGCTTCGAGGGGCTCGCATCTCTC
CTTCACGCGCCCGCCGCCCTACCTGAGGCCGCCATCCACGCCGGTTGAGTCGCGTTCT
GCCGCCTCCCGCCTGTGGTGCCTCCTGAACTGCGTCCGCCGTCTAGGTAAGTTTAAAG
CTCAGGTCGAGACCGGGCCTTTGTCCGGCGCTCCCTTGGAGCCTACCTAGACTCAGCC
GGCTCTCCACGCTTTGCCTGACCCTGCTTGCTCAACTCTACGTCTTTGTTTCGTTTTCT
GTTCTGCGCCGTTACAGATCCAAGCTGTGACCGGCGCCTACGATATCGCCACCATGAT
TAAGATCGCTACGCGGAAGTACCTGGGGAAACAGAACGTCTACGACATAGGTGTGGA
GCGCGATCACAACTTTGCTCTGAAAAATGGATTTATCGCCAGCAACTGTAGGGAGTT
GATTTCAGACTATGCACCAGATTGTTTTGTGTTAATAGAAATGAATGGCAAGTTAGTC
AAAACTACGATTGAAGAACTCATTCCACTCAAATATACCCGAAATTAAGTGCATGAC
CCGCAAGCCCGGTGCCTGAAATCAACCTCTGGATTACAAAATTTGTGAAAGATTGAC
TGGTATTCTTAACTATGTTGCTCCTTTTACGCTATGTGGATACGCTGCTTTAATGCCTT
TGTATCATGCGTTAACTAAACTTGTTTATTGCAGCTTATAATGGTTACAAATAAAGCA
ATAGCATCACAAATTTCACAAATAAAGCATTTTTTTCACTGCATTCTAGTTGTGGTTT
GTCCAAACTCATCAATGTATCTTATCATGTCTGGAATTGACTCAAATGATGTCAATTA
GTCTATCAGAAGCTCATCTGGTCTCCCTTCCGGGGGACAAGACATCCCTGTTTAATAT
TTAAACAGCAGTGTTCCCAAACTGGGTTCTTATATCCCTTGCTCTGGTCAACCAGGTT
GCAGGGTTTCCTGTCCTCACAGGAACGAAGTCCCTAAAGAAACAGTGGCAGCCAGGT
TTAGCCCCGGAATTGACTGGATTCCTTTTTTAGGGCCCATTGGTATGGCGATATCTAT
AACAAGAAAATATATATATAATAAGTTATCACGTAAGTAGAACATGAAATAACAATA
TAATTATCGTATGAGTTAAATCTTAAAAGTCACGTAAAAGATAATCATGCGTCATTTT
GACTCACGCGGTCGTTATAGTTCAAAATCAGTGACACTTACCGCATTGACAAGCACG
CCTCACGGGAGCTCCAAGCGGCGACTGAGATGTCCTAAATGCACAGCGACGGATTCG
CGCTATTTAGAAAGAGAGAGCAATATTTCAAGAATGCATGCGTCAATTTTACGCAGA
CTATCTTTCTAGGGTTAATCTAGCTGCATCAGGATCATATCGTCGGGTCTTTTTTCCGG
CTCAGTCATCGCCCAAGCTGGCGCTATCTGGGCATCGGGGAGGAAGAAGCCCGTGCC
TTTTCCCGCGAGGTTGAAGCGGCATGGAAAGAGTTTGCCGAGGATGACTGCTGCTGC
ATTGACGTTGAGCGAAAACGCACGTTTACCATGATGATTCGGGAAGGTGTGGCCATG
CACGCCTTTAACGGTGAACTGTTCGTTCAGGCCACCTGGGATACCAGTTCGTCGCGGC
TTTTCCGGACACAGTTCCGGATGGTCAGCCCGAAGCGCATCAGCAACCCGAACAATA
CCGGCGACAGCCGGAACTGCCGTGCCGGTGTGCAGATTAATGACAGCGGTGCGGCGC
TGGGATATTACGTCAGCGAGGACGGGTATCCTGGCTGGATGCCGCAGAAATGGACAT
GGATACCCCGTGAGTTACCCGGCGGGCGCGCTTGGCGTAATCATGGTCATAGCTGTTT
CCTGTGTGAAATTGTTATCCGCTCACAATTCCACACAACATACGAGCCGGAAGCATA
AAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCTGCATTAATGAATCGGCC
AACGCGCGGGGAGAGGCGGTTTGCGTATTGGGCGCTCTTCCGCTTCCTCGCTCACTGA
CTCGCTGCGCTCGGTCGTTCGGCTGCGGCGAGCGGTATCAGCTCACTCAAAGGCGGT
AATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGC
TCCGCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTCTCC
TGTTCCGACCCTGCCGCTTACCGGATACCTGTCCGCCTTTCTCCCTTCGGGAAGCGTG
GCGCTTTCTCATAGCTCACGCTGTAGGTATCTCAGTTCGGTGTAGGTCGTTCGCTCCA
AGCTGGGCTGTGTGCACGAACCCCCCGTTCAGCCCGACCGCTGCGCCTTATCCGGTAA
CTATCGTCTTGAGTCCAACCCGGTAAGACACGACTTATCGCCACTGGCAGCAGCCACT
GGTAACAGGATTAGCAGAGCGAGGTATGTAGGCGGTGCTACAGAGTTCTTGAAGTGG
TGGCCTAACTACGGCTACACTAGAAGGACAGTATTTGGTATCTGCGCTCTGCTGAAGC
CAGTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATCTC
AAGAAGATCCTTTGATCTTTTCTACGGGGTCTGACGCTCAGTGGAACGAAAACTCAC
GTTAAGGGATTTTGGTCATGAGATTATCAAAAAGGATCTTCACCTAGATCCTTTTAAA
TTAAAAATGAAGTTTTAAATCAATCTAAAGTATATATGAGTAAACTTGGTCTGACAGT
TACCAATGCTTAATCAGTGAGGCACCTATCTCAGCGATCTGTCTATTTCGTTCATCCA
TAGTTGCCTGACTCCCCGTCGTGTAGATAACTACGATACGGGAGGGCTTACCATCTGG
CCCCAGTGCTGCAATGATACCGCGAGACCCACGCTCACCGGCTCCAGATTTATCAGC
AATAAACCAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGCCAGTTAAT
AGTTTGCGCAACGTTGTTGCCATTGCTACAGGCATCGTGGTGTCACGCTCGTCGTTTG
GTATGGCTTCATTCAGCTCCGGTTCCCAACGATCAAGGCGAGTTACATGATCCCCCAT
GTTGTGCAAAAAAGCGGTTAGCTCCTTCGGTCCTCCGATCGTTGTCAGAAGTAAGTTG
GCCGCAGTGTTATCACTCATGGTTATGGCAGCACTGCATAATTCTCTTACTGTCATGC
CATCCGTAAGATGCTTTTCTGTGACTGGTGAGTACTCAACCAAGTCATTCTGAGAATA
GTGTATGCGGCGACCGAGTTGCTCTTGCCCGGCGTCAATACGGGATAATACCGCGCC
ACATAGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACCCAAC
TGATCTTCAGCATCTTTTACTTTCACCAGCGTTTCTGGGTGAGCAAAAACAGGAAGGC
AAAATGCCGCAAAAAAGGGAATAAGGGCGACACGGAAATGTTGAATACTCAT
SEQ ID NO: 33 (STXC0136)
ACTCTTCCTTTTTCAATATTATTGAAGCATTTATCAGGGTTATTGTCTCAT
GAGCGGATACATATTTGAATGTATTTAGAAAAATAAACAAATAGGGGTTCCGCGCAC
ATTTCCCCGAAAAGTGCCACCTAAATTGTAAGCGTTAATATTTTGTTAAAATTCGCGT
TAAATTTTTGTTAAATCAGCTCATTTTTTAACCAATAGGCCGAAATCGGCAAAATCCC
TTATAAATCAAAAGAATAGACCGAGATAGGGTTGAGTGTTGTTCCAGTTTGGAACAA
GAGTCCACTATTAAAGAACGTGGACTCCAACGTCAAAGGGCGAAAAACCGTCTATCA
GGGCGATGGCCCACTACGTGAACCATCACCCTAATCAAGTTTTTTGGGGTCGAGGTG
CCGTAAAGCACTAAATCGGAACCCTAAAGGGAGCCCCCGATTTAGAGCTTGACGGGG
AAAGCCGGCGAACGTGGCGAGAAAGGAAGGGAAGAAAGCGAAAGGAGCGGGCGCT
AGGGCGCTGGCAAGTGTAGCGGTCACGCTGCGCGTAACCACCACACCCGCCGCGCTT
AATGCGCCGCTACAGGGCGCGTCCCATTCGCCATTCAGGCTGCGCAACTGTTGGGAA
GGGCGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAAACG
ACGGCCAGTGAGCGCGCCTCGTTCATTCACGTTTTTGAACCCGTGGAGGACGGGCAG
ACTCGCGGTGCAAATGTGTTTTACAGCGTGATGGAGCAGATGAAGATGCTCGACACG
CTGCAGAACACGCAGCTAGATTAACCCTAGAAAGATAATCATATTGTGACGTACGTT
AAAGATAATCATGTGTAAAATTGACGCATGTGTTTTATCGGTCTGTATATCGAGGTTT
ATTTATTAATTTGAATAGATATTAAGTTTTATTATATTTACACTTACATACTAATAATA
AATTCAACAAACAATTTATTTATGTTTATTTATTTATTAAAAAAAACAAAAACTCAAA
ATTTCTTCTATAAAGTAACAAAACTTTTATTTCTTTCCTGCGTTATCCCCTGATTCTGT
GGATAACCGTATTACCGCCTTTGAGTGAGCTGATACCGCTCGCCGCAGCCGAACGAC
CGAGCGCAGCGAGTCAGTGAGCGAGGAAGCGGAAGAGCGCCCAATACGCAAACCGC
CTCTCCCCGCGCGTTGGCCGATTCATTAATGCAGCTGGCACGACAGGTTTCCCGACTG
GAAAGCGGGCAGTGAGCGCAACGCAATTAATGTGAGTTAGCTCACTCATTAGGCACC
CCAGGCTTTACACTTTATGCTTCCGGCTCGTATGTTGTGTGGAATTGTGAGCGGATAA
CAATTTCACACAGGAAACAGCTATGACCATGATTACGCCAAGCTTTTGGCCACTCCCT
CTCTGCGCGCTCGCTCGCTCACTGAGGCCGGGCGACCAAAGGTCGCCCGACGCCCGG
GCTTTGCCCGGGCGGCCTCAGTGAGCGAGCGAGCGCGCAGAGAGGGAGTGGCCAAC
TCCATCACTAGGGGTTCCTCTGCAGCCGCGACCGGCCAAGGTTTAATGATAGGCTGC
AACGGGATGTTGGGAATATGTTGCACTGGTCCGTGAGGGTACCAACTTGTTTATTGCA
GCTTATAATGGTTACAAATAAAGCAATAGCATCACAAATTTCACAAATAAAGCATTT
TTTTCACTGCATTCTAGTTGTGGTTTGTCCAAACTCATCAATGTATCTTATCATGTCTG
ACCGGTTCACTTGAGCTCGAGATCTGAGTACTTGTACAGCTCGTCCATGCCGAGAGTG
ATCCCGGCGGCGGTCACGAACTCCAGCAGGACCATGTGATCGCGCTTCTCGTTGGGG
TCTTTGCTCAGGGCGGACTGGGTGCTCAGGTAGTGGTTGTCGGGCAGCAGCACGGGG
CCGTCGCCGATGGGGGTGTTCTGCTGGTAGTGGTCGGCGAGCTGCACGCTGCCGTCCT
CGATGTTGTGGCGGATCTTGAAGTTCACCTTGATGCCGTTCTTCTGCTTGTCGGCCAT
GATATAGACGTTGTGGCTGTTGTAGTTGTACTCCAGCTTGTGCCCCAGGATGTTGCCG
TCCTCCTTGAAGTCGATGCCCTTCAGCTCGATGCGGTTCACCAGGGTGTCGCCCTCGA
ACTTCACCTCGGCGCGGGTCTTGTAGTTGCCGTCGTCCTTGAAGAAGATGGTGCGCTC
CTGGACGTAGCCTTCGGGCATGGCGGACTTGAAGAAGTCGTGCTGCTTCATGTGGTC
GGGGTAGCGGCTGAAGCACTGCACGCCGTAGGTCAGGGTGGTCACGAGGGTGGGCC
AGGGCACGGGCAGCTTGCCGGTGGTGCAGATGAACTTCAGGGTCAGCTTGCCGTAGG
TGGCATCGCCCTCGCCCTCGCCGGACACGCTGAACTTGTGGCCGTTTACGTCGCCGTC
CAGCTCGACCAGGATGGGCACCACCCCGGTGAACAGCTCCTCGCCCTTGCTCACCAT
GGTGGCGGCTTAAGGGTTCGATCCTCTAGAGTCCGGAGGCTGGATCGGTCCCGGTGT
CTACTATGGAGGTCAAAACAGCGTGGATGGCGTCTCCAGGCGATCTGACGGTTCACT
AAACGAGCTCTGCTTATATAGACCTCCCACCGTACACGCCTACCGCCCATTTGCGTCA
ATGGGGCGGAGTTGTTACGACATTTTGGAAAGTCCCGTTGATTTTGGTGCCAAAACA
AACTCCCATTGACGTCAATGGGGTGGAGACTTGGAAATCCCCGTGAGTCAAACCGCT
ATCCACGCCCATTGATGTACTGCCAAAACCGCATCACCATGGTAATAGCGATGACTA
ATACGTAGATGTACTGCCAAGTAGGAAAGTCCCATAAGGTCATGTACTGGGCATAAT
GCCAGGCGGGCCATTTACCGTCATTGACGTCAATAGGGGGCGTACTTGGCATATGAT
ACACTTGATGTACTGCCAAGTGGGCAGTTTACCGTAAATACTCCACCCATTGACGTCA
ATGGAAAGTCCCTATTGGCGTTACTATGGGAACATACGTCATTATTGACGTCAATGGG
CGGGGGTCGTTGGGCGGTCAGCCAGGCGGGCCATTTACCGTAAGTTATGTAACGCGG
AACTCCATATATGGGCTATGAACTAATGACCCCGTAATTGATTACTATTAATAACTAG
TCAATAATCAATGTCAACGCGTATGGTACCTGCGGAGGATGCCGAGGATAACCTTGT
TACTAGCCTCCGCCTGGCCGTTGGACTGTGGATAATATGGCGTAGAGGATCCTCTGCG
CGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAATTCACTGGCCGTCGTTTTAC
AACGTCGTGACTGGGAAAACCCTGGCGTTACCCAACTTAATCGCCTTGCAGCACATC
CCCCTTTCGCCAGCTGGCGTAATAGCGAAGAGGCCCGCACCGATCGCCCTTCCCAAC
AGTTGCGCAGCCTGAATGGCGAATGGTCTAGAGCTAGCGAATTCGAATTTAAATCGG
ATCCGCGGCCGCAAGGATCTGCGATCGCTCCGGTGCCCGTCAGTGGGCAGAGCGCAC
ATCGCCCACAGTCCCCGAGAAGTTGGGGGGAGGGGTCGGCAATTGAACGGGTGCCTA
GAGAAGGTGGCGCGGGGTAAACTGGGAAAGTGATGTCGTGTACTGGCTCCGCCTTTT
TCCCGAGGGTGGGGGAGAACCGTATATAAGTGCAGTAGTCGCCGTGAACGTTCTTTT
TCGCAACGGGTTTGCCGCCAGAACACAGCTGAAGCTTCGAGGGGCTCGCATCTCTCC
TTCACGCGCCCGCCGCCCTACCTGAGGCCGCCATCCACGCCGGTTGAGTCGCGTTCTG
CCGCCTCCCGCCTGTGGTGCCTCCTGAACTGCGTCCGCCGTCTAGGTAAGTTTAAAGC
TCAGGTCGAGACCGGGCCTTTGTCCGGCGCTCCCTTGGAGCCTACCTAGACTCAGCCG
GCTCTCCACGCTTTGCCTGACCCTGCTTGCTCAACTCTACGTCTTTGTTTCGTTTTCTGT
TCTGCGCCGTTACAGATCCAAGCTGTGACCGGCGCCTACGATATCGCCACCATGAAA
ACATTTAACATTTCTCAACAGGATCTAGAATTAGTAGAAGTAGCGACAGAGAAGATT
ACAATGCTTTATGAGGATAATAAACATCATGTGGGAGCGGCAATTCGTACGAAAACA
GGAGAAATCATTTCGGCAGTACATATTGAAGCGTATATAGGACGAGTAACTGTTTGT
GCAGAAGCCATTGCGATTGGTAGTGCAGTTTCGAATGGACAAAAGGATTTTGACACG
ATTGTAGCTGTTAGACACCCTTATTCTGACGAAGTAGATAGAAGTATTCGAGTGGTAA
GTCCTTGTGGTATGTGCCTTTCATACGAGACCGAGATCCTGACTGTCGAGTACGGATT
GCTTCCTATCGGCAAAATCGTGGAGAAGAGGATTGAATGTACCGTCTATTCAGTCGA
TAATAATGGGAACATCTACACACAGCCCGTGGCTCAATGGCACGACAGAGGAGAGC
AGGAAGTTTTTGAATACTGTCTCGAGGACGGATCCCTCATCCGCGCTACTAAAGATCA
TAAGTTTATGACCGTGGACGGCCAGATGCTGCCAATTGACGAAATTTTTGAACGAGA
GCTGGATCTGATGAGAGTCGACAACCTTCCAAACTGAGTGCATGACCCGCAAGCCCG
GTGCCTGAAATCAACCTCTGGATTACAAAATTTGTGAAAGATTGACTGGTATTCTTAA
CTATGTTGCTCCTTTTACGCTATGTGGATACGCTGCTTTAATGCCTTTGTATCATGCGT
TAACTAAACTTGTTTATTGCAGCTTATAATGGTTACAAATAAAGCAATAGCATCACAA
ATTTCACAAATAAAGCATTTTTTTCACTGCATTCTAGTTGTGGTTTGTCCAAACTCATC
AATGTATCTTATCATGTCTGGAATTGACTCAAATGATGTCAATTAGTCTATCAGAAGC
TATCTGGTCTCCCTTCCGGGGGACAAGACATCCCTGTTTAATATTTAAACAGCAGTGT
TCCCAAACTGGGTTCTTATATCCCTTGCTCTGGTCAACCAGGTTGCAGGGTTTCCTGTC
CTCACAGGAACGAAGTCCCTAAAGAAACAGTGGCAGCCAGGTTTAGCCCCGGAATTG
ACTGGATTCCTTTTTTAGGGCCCATTGGTATGGCTGATATCTATAACAAGAAAATATA
TATATAATAAGTTATCACGTAAGTAGAACATGAAATAACAATATAATTATCGTATGA
GTTAAATCTTAAAAGTCACGTAAAAGATAATCATGCGTCATTTTGACTCACGCGGTCG
TTATAGTTCAAAATCAGTGACACTTACCGCATTGACAAGCACGCCTCACGGGAGCTC
CAAGCGGCGACTGAGATGTCCTAAATGCACAGCGACGGATTCGCGCTATTTAGAAAG
AGAGAGCAATATTTCAAGAATGCATGCGTCAATTTTACGCAGACTATCTTTCTAGGGT
TAATCTAGCTGCATCAGGATCATATCGTCGGGTCTTTTTTCCGGCTCAGTCATCGCCC
AAGCTGGCGCTATCTGGGCATCGGGGAGGAAGAAGCCCGTGCCTTTTCCCGCGAGGT
TGAAGCGGCATGGAAAGAGTTTGCCGAGGATGACTGCTGCTGCATTGACGTTGAGCG
AAAACGCACGTTTACCATGATGATTCGGGAAGGTGTGGCCATGCACGCCTTTAACGG
TGAACTGTTCGTTCAGGCCACCTGGGATACCAGTTCGTCGCGGCTTTTCCGGACACAG
TTCCGGATGGTCAGCCCGAAGCGCATCAGCAACCCGAACAATACCGGCGACAGCCGG
AACTGCCGTGCCGGTGTGCAGATTAATGACAGCGGTGCGGCGCTGGGATATTACGTC
AGCGAGGACGGGTATCCTGGCTGGATGCCGCAGAAATGGACATGGATACCCCGTGAG
TTACCCGGCGGGCGCGCTTGGCGTAATCATGGTCATAGCTGTTTCCTGTGTGAAATTG
TTATCCGCTCACAATTCCACACAACATACGAGCCGGAAGCATAAAGTGTAAAGCCTG
GGGTGCCTAATGAGTGAGCTAACTCACATTAATTGCGTTGCGCTCACTGCCCGCTTTC
CAGTCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTCGG
TCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCC
AGGAACCGTAAAAAGGCCGCGTTGCTGGCGTTTTTCCATAGGCTCCGCCCCCCTGAC
GAGCATCACAAAAATCGACGCTCAAGTCAGAGGTGGCGAAACCCGACAGGACTATA
AAGATACCAGGCGTTTCCCCCTGGAAGCTCCCTCGTGCGCTCTCCTGTTCCGACCCTG
CCGCTTACCGGATACCTGTCCGCCTTTCTCCCTTCGGGAAGCGTGGCGCTTTCTCATA
GCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAG
TCCAACCCGGTAAGACACGACTTATCGCCACTGGCAGCAGCCACTGGTAACAGGATT
AGCAGAGCGAGGTATGTAGGCGGTGCTACAGAGTTCTTGAAGTGGTGGCCTAACTAC
GGCTACACTAGAAGGACAGTATTTGGTATCTGCGCTCTGCTGAAGCCAGTTACCTTCG
GAAAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCCTT
TGATCTTTTCTACGGGGTCTGACGCTCAGTGGAACGAAAACTCACGTTAAGGGATTTT
GGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAA
TCAGTGAGGCACCTATCTCAGCGATCTGTCTATTTCGTTCATCCATAGTTGCCTGACTC
CCCGTCGTGTAGATAACTACGATACGGGAGGGCTTACCATCTGGCCCCAGTGCTGCA
ATGATACCGCGAGACCCACGCTCACCGGCTCCAGATTTATCAGCAATAAACCAGCCA
GCCGGAAGGGCCGAGCGCAGAAGTGGTCCTGCAACTTTATCCGCCTCCATCCAGTCT
ATTAATTGTTGCCGGGAAGCTAGAGTAAGTAGTTCGCCAGTTAATAGTTTGCGCAAC
GTTGTTGCCATTGCTACAGGCATCGTGGTGTCACGCTCGTCGTTTGGTATGGCTTCATT
CAGCTCCGGTTCCCAACGATCAAGGCGAGTTACATGATCCCCCATGTTGTGCAAAAA
AGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGAT
GCTTTTCTGTGACTGGTGAGTACTCAACCAAGTCATTCTGAGAATAGTGTATGCGGCG
ACCGAGTTGCTCTTGCCCGGCGTCAATACGGGATAATACCGCGCCACATAGCAGAAC
TTTAAAAGTGCTCATCATTGGAAAACGTTCTTCGGGGCGAAAACTCTCAAGGATCTTA
CCGCTGTTGAGATCCAGTTCGATGTAACCCACTCGTGCACCCAACTGATCTTCAGCAT
CTTTTACTTTCACCAGCGTTTCTGGGTGAGCAAAAACAGGAAGGCAAAATGCCGCAA
AAAAGGGAATAAGGGCGACACGGAAATGTTGAATACTCAT
SEQ ID NO: 34 (STX650)
TCGCGCGTTTCGGTGATGACGGTGAAAACCTCTGACACATGCAGCTCCCG
GAGACGGTCACAGCTTGTCTGTAAGCGGATGCCGGGAGCAGACAAGCCCGTCAGGGC
GCGTCAGCGGGTGTTGGCGGGTGTCGGGGCTGGCTTAACTATGCGGCATCAGAGCAG
ATTGTACTGAGAGTGCACCATATGCGGTGTGAAATACCGCACAGATGCGTAAGGAGA
AAATACCGCATCAGGCGCCATTCGCCATTCAGGCTGCGCAACTGTTGGGAAGGGCGA
TCGGTGCGGGCCTCTTCGCTATTACGCCAGCTGGCGAAAGGGGGATGTGCTGCAAGG
CGATTAAGTTGGGTAACGCCAGGGTTTTCCCAGTCACGACGTTGTAAAACGACGGCC
AGTGAATTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGGATCCTC
TACGCCATATTATCCACAGTCCAACGGCCAGGCGGAGGCTAGTAACAAGGTTATCCT
CGGCATCCTCCGCAGGTACCATACGCGTTGACATTGATTATTGACTAGTTATTAATAG
TAATCAATTACGGGGTCATTAGTTCATAGCCCATATATGGAGTTCCGCGTTACATAAC
TTACGGTAAATGGCCCGCCTGGCTGACCGCCCAACGACCCCCGCCCATTGACGTCAA
TAATGACGTATGTTCCCATAGTAACGCCAATAGGGACTTTCCATTGACGTCAATGGGT
GGAGTATTTACGGTAAACTGCCCACTTGGCAGTACATCAAGTGTATCATATGCCAAGT
ACGCCCCCTATTGACGTCAATGACGGTAAATGGCCCGCCTGGCATTATGCCCAGTAC
ATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCAC
GGGGATTTCCAAGTCTCCACCCCATTGACGTCAATGGGAGTTTGTTTTGGCACCAAAA
TCAACGGGACTTTCCAAAATGTCGTAACAACTCCGCCCCATTGACGCAAATGGGCGG
TAGGCGTGTACGGTGGGAGGTCTATATAAGCAGAGCTCGTTTAGTGAACCGTCAGAT
CGCCTGGAGACGCCATCCACGCTGTTTTGACCTCCATAGTAGACACCGGGACCGATC
CAGCCTCCGGACTCTAGAGGATCGAACCCTTAAGCCGCCACCATGGTGAGCAAGGGC
GAGGAGCTGTTCACCGGGGTGGTGCCCATCCTGGTCGAGCTGGACGGCGACGTAAAC
GGCCACAAGTTCAGCGTGTCCGGCGAGGGCGAGGGCGATGCCACCTACGGCAAGCTG
ACCCTGAAGTTCATCTGCACCACCGGCAAGCTGCCCGTGCCCTGGCCCACCCTCGTGA
CCACCCTGACCTACGGCGTGCAGTGCTTCAGCCGCTACCCCGACCACATGAAGCAGC
ACGACTTCTTCAAGTCCGCCATGCCCGAAGGCTACGTCCAGGAGCGCACCATCTTCTT
CAAGGACGACGGCAACTACAAGACCCGCGCCGAGGTGAAGTTCGAGGGCGACACCC
TGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGC
AGAAGAACGGCATCAAGGTGAACTTCAAGATCCGCCACAACATCGAGGACGGCAGC
GTGCAGCTCGCCGACCACTACCAGCAGAACACCCCCATCGGCGACGGCCCCGTGCTG
CTGCCCGACAACCACTACCTGAGCACCCAGTCCGCCCTGAGCAAAGACCCCAACGAG
AAGCGCGATCACATGGTCCTGCTGGAGTTCGTGACCGCCGCCGGGATCACTCTCGGC
ATGGACGAGCTGTACAAGTACTCAGATCTCGAGCTCAAGTGAACCGGTCAGACATGA
TAAGATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAATAAA
CAAGTTGGTACCCTCACGGACCAGTGCAACATATTCCCAACATCCCGTTGCAGCCTAT
CATTAAACCTTGGCCGGTCGCGGCTGCAGAGGAACCCCTAGTGATGGAGTTGGCCAC
TCCCTCTCTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGGGAGTGG
CCAAAAGCTTGGCGTAATCATGGTCATAGCTGTTTCCTGTGTGAAATTGTTATCCGCT
CACAATTCCACACAACATACGAGCCGGAAGCATAAAGTGTAAAGCCTGGGGTGCCTA
ATGAGTGAGCTAACTCACATTAATTGCGTTGCGCTCACTGCCCGCTTTCCAGTCGGGA
AACCTGTCGTGCCAGCTGCATTAATGAATCGGCCAACGCGCGGGGAGAGGCGGTTTG
CGTATTGGGCGCTCTTCCGCTTCCTCGCTCACTGACTCGCTGCGCTCGGTCGTTCGGCT
GCGGCGAGCGGTATCAGCTCACTCAAAGGCGGTAATACGGTTATCCACAGAATCAGG
GGATAACGCAGGAAAGAACATGTGAGCAAAAGGCCAGCAAAAGGCCAGGAACCGTA
AAAAGGCCGCGTTGCTGGCGTTTTTCCATAGGCTCCGCCCCCCTGACGAGCATCACAA
AAATCGACGCTCAAGTCAGAGGTGGCGAAACCCGACAGGACTATAAAGATACCAGG
CGTTTCCCCCTGGAAGCTCCCTCGTGCGCTCTCCTGTTCCGACCCTGCCGCTTACCGG
ATACCTGTCCGCCTTTCTCCCTTCGGGAAGCGTGGCGCTTTCTCATAGCTCACGCTGT
AGGTATCTCAGTTCGGTGTAGGTCGTTCGCTCCAAGCTGGGCTGTGTGCACGAACCCC
CCGTTCAGCCCGACCGCTGCGCCTTATCCGGTAACTATCGTCTTGAGTCCAACCCGGT
AAGACACGACTTATCGCCACTGGCAGCAGCCACTGGTAACAGGATTAGCAGAGCGAG
GTATGTAGGCGGTGCTACAGAGTTCTTGAAGTGGTGGCCTAACTACGGCTACACTAG
AAGAACAGTATTTGGTATCTGCGCTCTGCTGAAGCCAGTTACCTTCGGAAAAAGAGT
TGGTAGCTCTTGATCCGGCAAACAAACCACCGCTGGTAGCGGTGGTTTTTTTGTTTGC
AAGCAGCAGATTACGCGCAGAAAAAAAGGATCTCAAGAAGATCCTTTGATCTTTTCT
ACGGGGTCTGACGCTCAGTGGAACGAAAACTCACGTTAAGGGATTTTGGTCATGAGA
TTATCAAAAAGGATCTTCACCTAGATCCTTTTAAATTAAAAATGAAGTTTTAAATCAA
TCTAAAGTATATATGAGTAAACTTGGTCTGACAGTTACCAATGCTTAATCAGTGAGGC
ACCTATCTCAGCGATCTGTCTATTTCGTTCATCCATAGTTGCCTGACTCCCCGTCGTGT
AGATAACTACGATACGGGAGGGCTTACCATCTGGCCCCAGTGCTGCAATGATACCGC
GAGACCCACGCTCACCGGCTCCAGATTTATCAGCAATAAACCAGCCAGCCGGAAGGG
CCGAGCGCAGAAGTGGTCCTGCAACTTTATCCGCCTCCATCCAGTCTATTAATTGTTG
CCGGGAAGCTAGAGTAAGTAGTTCGCCAGTTAATAGTTTGCGCAACGTTGTTGCCATT
GCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCT
CCTTCGGTCCTCCGATCGTTGTCAGAAGTAAGTTGGCCGCAGTGTTATCACTCATGGT
TATGGCAGCACTGCATAATTCTCTTACTGTCATGCCATCCGTAAGATGCTTTTCTGTG
ACTGGTGAGTACTCAACCAAGTCATTCTGAGAATAGTGTATGCGGCGACCGAGTTGC
TCTTGCCCGGCGTCAATACGGGATAATACCGCGCCACATAGCAGAACTTTAAAAGTG
CTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAAT
AAGGGCGACACGGAAATGTTGAATACTCATACTCTTCCTTTTTCAATATTATTGAAGC
ATTTATCAGGGTTATTGTCTCATGAGCGGATACATATTTGAATGTATTTAGAAAAATA
AACAAATAGGGGTTCCGCGCACATTTCCCCGAAAAGTGCCACCTGACGTCTAAGAAA
CCATTATTATCATGACATTAACCTATAAAAATAGGCGTATCACGAGGCCCTTTCGTC
SEQ ID NO: 35 (STXC002)
ggtacccaactccatgcttaacagtccccaggtacagcccaccctgcgtcgcaaccaggaacagctctacagcttcctgg
agcgccactcgccctacttccgcagccacagtgcgcagattaggagcgccacttctttttgtcacttgaaaaacatgtaaaaataatgtactagg
agacactttcaataaaggcaaatgtttttatttgtacactctcgggtgattatttaccccccacccttgccgtctgcgccgtttaaaaatcaaaggg
gttctgccgcgcatcgctatgcgccactggcagggacacgttgcgatactggtgtttagtgctccacttaaactcaggcacaaccatccgcgg
cagctcggtgaagttttcactccacaggctgcgcaccatcaccaacgcgtttagcaggtcgggcgccgatatcttgaagtcgcagttggggc
ctccgccctgcgcgcgcgagttgcgatacacagggttgcagcactggaacactatcagcgccgggtggtgcacgctggccagcacgctctt
gtcggagatcagatccgcgtccaggtcctccgcgttgctcagggcgaacggagtcaactttggtagctgccttcccaaaaagggtgcatgcc
caggctttgagttgcactcgcaccgtagtggcatcagaaggtgaccgtgcccggtctgggcgttaggatacagcgcctgcatgaaagccttg
atctgcttaaaagccacctgagcctttgcgccttcagagaagaacatgccgcaagacttgccggaaaactgattggccggacaggccgcgtc
atgcacgcagcaccttgcgtcggtgttggagatctgcaccacatttcggccccaccggttcttcacgatcttggccttgctagactgctccttca
gcgcgcgctgcccgttttcgctcgtcacatccatttcaatcacgtgctccttatttatcataatgctcccgtgtagacacttaagctcgccttcgatc
tcagcgcagcggtgcagccacaacgcgcagcccgtgggctcgtggtgcttgtaggttacctctgcaaacgactgcaggtacgcctgcagga
atcgccccatcatcgtcacaaaggtcttgttgctggtgaaggtcagctgcaacccgcggtgctcctcgtttagccaggtcttgcatacggccgc
cagagcttccacttggtcaggcagtagcttgaagtttgcctttagatcgttatccacgtggtacttgtccatcaacgcgcgcgcagcctccatgc
ccttctcccacgcagacacgatcggcaggctcagcgggtttatcaccgtgctttcactttccgcttcactggactcttccttttcctcttgcgtccg
cataccccgcgccactgggtcgtcttcattcagccgccgcaccgtgcgcttacctcccttgccgtgcttgattagcaccggtgggttgctgaaa
cccaccatttgtagcgccacatcttctctttcttcctcgctgtccacgatcacctctggggatgggggcgctcgggcttgggagaggggcgct
tctttttctttttggacgcaatggccaaatccgccgtcgaggtcgatggccgcgggctgggtgtgcgcggcaccagcgcatcttgtgacgagt
cttcttcgtcctcggactcgagacgccgcctcagccgcttttttgggggcgcgcggggaggcggcggcgacggcgacggggacgacacg
tcctccatggttggtggacgtcgcgccgcaccgcgtccgcgctcgggggtggtttcgcgctgctcctcttcccgactggccatttccttctccta
taggcagaaaaagatcatggagtcagtcgagaaggaggacagcctaaccgccccctttgagttcgccaccaccgcctccaccgatgccgc
caacgcgcctaccaccttccccgtcgaggcacccccgcttgaggaggaggaagtgattatcgagcaggacccaggttttgtaagcgaagac
gacgaggatcgctcagtaccaacagaggataaaaagcaagaccaggacgacgcagaggcaaacgaggaacaagtcgggcgggggga
ccaaaggcatggcgactacctagatgtgggagacgacgtgctgttgaagcatctgcagcgccagtgcgccattatctgcgacgcgttgcaa
gagcgcagcgatgtgcccctcgccatagcggatgtcagccttgcctacgaacgccacctgttctcaccgcgcgtaccccccaaacgccaag
aaaacggcacatgcgagcccaacccgcgcctcaacttctaccccgtatttgccgtgccagaggtgcttgccacctatcacatctttttccaaaa
ctgcaagatacccctatcctgccgtgccaaccgcagccgagcggacaagcagctggccttgcggcagggcgctgtcatacctgatatcgcc
tcgctcgacgaagtgccaaaaatctttgagggtcttggacgcgacgagaaacgcgcggcaaacgctctgcaacaagaaaacagcgaaaat
gaaagtcactgtggagtgctggtggaacttgagggtgacaacgcgcgcctagccgtgctgaaacgcagcatcgaggtcacccactttgcct
acccggcacttaacctaccccccaaggttatgagcacagtcatgagcgagctgatcgtgcgccgtgcacgacccctggagagggatgcaa
acttgcaagaacaaaccgaggagggcctacccgcagttggcgatgagcagctggcgcgctggcttgagacgcgcgagcctgccgacttg
gaggagcgacgcaagctaatgatggccgcagtgcttgttaccgtggagcttgagtgcatgcagcggttctttgctgacccggagatgcagcg
caagctagaggaaacgttgcactacacctttcgccagggctacgtgcgccaggcctgcaaaatttccaacgtggagctctgcaacctggtctc
ctaccttggaattttgcacgaaaaccgcctcgggcaaaacgtgcttcattccacgctcaagggcgaggcgcgccgcgactacgtccgcgact
gcgtttacttatttctgtgctacacctggcaaacggccatgggcgtgtggcagcaatgcctggaggagcgcaacctaaaggagctgcagaag
ctgctaaagcaaaacttgaaggacctatggacggccttcaacgagcgctccgtggccgcgcacctggggacattatottccccgaacgcct
gcttaaaaccctgcaacagggtctgccagacttcaccagtcaaagcatgttgcaaaactttaggaactttatcctagagcgttcaggaattctgc
ccgccacctgctgtgcgcttcctagcgactttgtgcccattaagtaccgtgaatgccctccgccgctttggggtcactgctaccttctgcagcta
gccaactaccttgcctaccactccgacatcatggaagacgtgagcggtgacggcctactggagtgtcactgtcgctgcaacctatgcacccc
gcaccgctccctggtctgcaattcgcaactgcttagcgaaagtcaaattatcggtacctttgagctgcagggtccctcgcctgacgaaaagtcc
gcggctccggggttgaaactcactccggggctgtggacgtcggcttaccttcgcaaatttgtacctgaggactaccacgcccacgagattag
gttctacgaagaccaatcccgcccgccaaatgcggagcttaccgcctgcgtcattacccagggccacatccttggccaattgcaagccatca
acaaagcccgccaagagtttctgctacgaaagggacggggggtttacctggacccccagtccggcgaggagctcaacccaatccccccgc
cgccgcagccctatcagcagccgcgggcccttgcttcccaggatggcacccaaaaagaagctgcagctgccgccgccgccacccacgga
cgaggaggaatactgggacagtcaggcagaggaggttttggacgaggaggaggagatgatggaagactgggacagcctagacgaagctt
ccgaggccgaagaggtgtcagacgaaacaccgtcaccctcggtcgcattcccctcgccggcgccccagaaattggcaaccgttcccagca
tcgctacaacctccgctcctcaggcgccgccggcactgcctgttcgccgacccaaccgtagatgggacaccactggaaccagggccggta
agtctaagcagccgccgccgttagcccaagagcaacaacagcgccaaggctaccgctcgtggcgcgggcacaagaacgccatagttgctt
gcttgcaagactgtgggggcaacatctccttcgcccgccgctttcttctctaccatcacggcgtggccttcccccgtaacatcctgcattactac
cgtcatctctacagcccctactgcaccggcggcagcggcagcggcagcaacagcagcggtcacacagaagcaaaggcgaccggatagc
aagactctgacaaagcccaagaaatccacagcggcggcagcagcaggaggaggagcgctgcgtctggcgcccaacgaacccgtatcga
cccgcgagcttagaaataggatttttcccactctgtatgctatatttcaacaaagcaggggccaagaacaagagctgaaaaaaaaaacaggt
ctctgcgctccctcacccgcagctgcctgtatcacaaaagcgaagatcagcttcggcgcacgctggaagacgcggaggctctcttcagcaa
atactgcgcgctgactcttaaggactagtttcgcgccctttctcaaatttaagcgcgaaaactacgtcatctccagcggccacacccggcgcca
gcacctgtcgtcagcgccattatgagcaaggaaattcccacgccctacatgtggagttaccagccacaaatgggacttgcggctggagctgc
ccaagactactcaacccgaataaactacatgagcgcgggaccccacatgatatcccgggtcaacggaatccgcgcccaccgaaaccgaatt
ctcctcgaacaggcggctattaccaccacacctcgtaataaccttaatccccgtagttggcccgctgccctggtgtaccaggaaagtcccgct
cccaccactgtggtacttcccagagacgcccaggccgaagttcagatgactaactcaggggcgcagcttgcgggcggctttcgtcacaggg
tgcggtcgcccgggcgttttagggcggagtaacttgcatgtattgggaattgtagtttttttaaaatgggaagtgacgtatcgtgggaaaacgga
agtgaagatttgaggaagttgtgggttttttggctttcgtttctgggcgtaggttcgcgtgcggttttctgggtgttttttgtggactttaaccgttacg
tcattttttagtcctatatatactcgctctgtacttggccctttttacactgtgactgattgagctggtgccgtgtcgagtggtgttttttaataggttttttt
actggtaaggctgactgttatggctgccgctgtggaagcgctgtatgttgttctggagcgggagggtgctattttgcctaggcaggagggttttt
caggtgtttatgtgtttttctctcctattaattttgttatacctcctatgggggctgtaatgttgtctctacgcctgcgggtatgtattcccccgggctatt
tcggtcgctttttagcactgaccgatgttaaccaacctgatgtgtttaccgagtcttacattatgactccggacatgaccgaggaactgtcggtgg
tgctttttaatcacggtgaccagtttttttacggtcacgccggcatggccgtagtccgtcttatgcttataagggttgtttttcctgttgtaagacagg
cttctaatgtttaaatgtttttttttttgttattttattttgtgtttaatgcaggaacccgcagacatgtttgagagaaaaatggtgtctttttctgtggtggtt
ccggaacttacctgcctttatctgcatgagcatgactacgatgtgcttgcttttttgcgcgaggctttgcctgattttttgagcagcaccttgcatttt
atatcgccgcccatgcaacaagcttacataggggctacgctggttagcatagctccgagtatgcgtgtcataatcagtgtgggttcttttgtcatg
gttcctggcggggaagtggccgcgctggtccgtgcagacctgcacgattatgttcagctggccctgcgaagggacctacgggatcgcggta
tttttgttaatgttccgcttttgaatcttatacaggtctgtgaggaacctgaatttttgcaatcatgattcgctgcttgaggctgaaggtggagggcg
ctctggagcagatttttacaatggccggacttaatattcgggatttgcttagagacatattgataaggtggcgagatgaaaattatttgggcatggt
tgaaggtgctggaatgtttatagaggagattcaccctgaagggtttagcctttacgtccacttggacgtgagggcagtttgccttttggaagccat
tgtgcaacatcttacaaatgccattatctgttctttggctgtagagtttgaccacgccaccggaggggagcgcgttcacttaatagatcttcattttg
aggttttggataatcttttggaataaaaaaaaaaaaacatggttcttccagctcttcccgctcctcccgtgtgtgactcgcagaacgaatgtgtag
gttggctgggtgtggcttattctgcggtggtggatgttatcagggcagcggcgcatgaaggagtttacatagaacccgaagccagggggcgc
ctggatgctttgagagagtggatatactacaactactacacagagcgagctaagcgacgagaccggagacgcagatctgtttgtcacgcccg
cacctggttttgcttcaggaaatatgactacgtccggcgttccatttggcatgacactacgaccaacacgatctcggttgtctcggcgcactccg
tacagtagggatcgcctacctccttttgagacagagacccgcgctaccatactggaggatcatccgctgctgcccgaatgtaacactttgacaa
tgcacaacgtgagttacgtgcgaggtcttccctgcagtgtgggatttacgctgattcaggaatgggttgttccctgggatatggttctgacgcgg
gaggagcttgtaatcctgaggaagtgtatgcacgtgtgcctgtgttgtgccaacattgatatcatgacgagcatgatgatccatggttacgagtc
ctgggctctccactgtcattgttccagtcccggttccctgcagtgcatagccggcgggcaggttttggccagctggtttaggatggtggtggat
ggcgccatgtttaatcagaggtttatatggtaccgggaggtggtgaattacaacatgccaaaagaggtaatgtttatgtccagcgtgtttatgag
gggtcgccacttaatctacctgcgcttgtggtatgatggccacgtgggttctgtggtccccgccatgagctttggatacagcgccttgcactgtg
ggattttgaacaatattgtggtgctgtgctgcagttactgtgctgatttaagtgagatcagggtgcgctgctgtgcccggaggacaaggcgtctc
atgctgcgggcggtgcgaatcatcgctgaggagaccactgccatgttgtattcctgcaggacggagcggcggcggcagcagtttattcgcg
cgctgctgcagcaccaccgccctatcctgatgcacgattatgactctacccccatgtaggcgtggacttccccttcgccgcccgttgagcaac
cgcaagttggacagcagcctgtggctcagcagctggacagcgacatgaacttaagcgagctgcccggggagtttattaatatcactgatgag
cgtttggctcgacaggaaaccgtgtggaatataacacctaagaatatgtctgttacccatgatatgatgctttttaaggccagccggggagaaa
ggactgtgtactctgtgtgttgggagggaggtggcaggttgaatactagggttctgtgagtttgattaaggtacggtgatcaatataagctatgtg
gtggtggggctatactactgaatgaaaaatgacttgaaattttctgcaattgaaaaataaacacgttgaaacataacatgcaacaggttcacgatt
ctttattcctgggcaatgtaggagaaggtgtaagagttggtagcaaaagtttcagtggtgtattttccactttcccaggaccatgtaaaagacata
gagtaagtgcttacctcgctagtttctgtggattcactagaatcgatgtcgacgtttaaaccatatgatcagctcactcaaaggcggtaatacggt
tatccacagaatcaggggataacgcaggaaagaacatgtgagcaaaaggccagcaaaaggccaggaaccgtaaaaaggccgcgttgctg
gcgtttttccataggctccgcccccctgacgagcatcacaaaaatcgacgctcaagtcagaggtggcgaaacccgacaggactataaagata
ccaggcgtttccccctggaagctccctcgtgcgctctcctgttccgaccctgccgcttaccggatacctgtccgcctttctcccttcgggaagcg
tggcgctttctcatagctcacgctgtaggtatctcagttcggtgtaggtcgttcgctccaagctgggctgtgtgcacgaaccccccgttcagccc
gaccgctgcgccttatccggtaactatcgtcttgagtccaacccggtaagacacgacttatcgccactggcagcagccactggtaacaggatt
agcagagcgaggtatgtaggcggtgctacagagttcttgaagtggtggcctaactacggctacactagaagaacagtatttggtatctgcgct
ctgctgaagccagttaccttcggaaaaagagttggtagctcttgatccggcaaacaaaccaccgctggtagcggtggtttttttgtttgcaagca
gcagattacgcgcagaaaaaaaggatctcaagaagatcctttgatcttttctacggggtctgacgctcagtggaacgaaaactcacgttaagg
gattttggtcatgagattatcaaaaaggatcttcacctagatccttttaaattaaaaatgaagttttaaatcaatctaaagtatatatgagtaaacttgg
tctgacagttaccaatgcttaatcagtgaggcacctatctcagcgatctgtctatttcgttcatccatagttgcctgactccccgtcgtgtagataac
tacgatacgggagggcttaccatctggccccagtgctgcaatgataccgcgagacccacgctcaccggctccagatttatcagcaataaacc
agccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctccgatcgttgtcagaagtaagttggcc
gcagtgttatcactcatggttatggcagcactgcataattctcttactgtcatgccatccgtaagatgcttttctgtgactggtgagtactcaaccaa
gtcattctgagaatagtgtatgcggcgaccgagttgctcttgcccggcgtcaatacgggataataccgcgccacatagcagaactttaaaagt
gctcatcattggaaaacgttcttcggggcgaaaactctcaaggatcttaccgctgttgagatccagttcgatgtaacccactcgtgcacccaact
gatcttcagcatcttttactttcaccagcgtttctgggtgagcaaaaacaggaaggcaaaatgccgcaaaaaagggaataagggcgacacgg
aaatgttgaatactcatactcttcctttttcaatattattgaagcatttatcagggttattgtctcatgagcggatacatatttgaatgtatttagaaaaat
aaacaaataggggttccgcgcacatttccccgaaaagtgccacctaaattgtaagcgttaatattttgttaaaattcgcgttaaatttttgttaaatc
agctcattttttaaccaataggccgaaatcggcaaaatcccttataaatcaaaagaatagaccgagatagggttgagtgttgttccagtttggaac
aagagtccactattaaagaacgtggactccaacgtcaaagggcgaaaaaccgtctatcagggcgatggcccactacgtgaaccatcaccct
aatcaagttttttggggtcgaggtgccgtaaagcactaaatcggaaccctaaagggagcccccgatttagagcttgacggggaaagccggc
gaacgtggcgagaaaggaagggaagaaagcgaaaggagcgggcgctagggcgctggcaagtgtagcggtcacgctgcgcgtaaccac
cacacccgccgcgcttaatgcgccgctacagggcgcgatggatcc

| SEQ ID | | |
|---|---|---|
| NO | DESCRIPTION | SEQUENCE |
| SEQ ID | Full-length | MSTAVLENPGLGRKLSDFGQETSYIEDNCNQNGAISLIFSLKEEVG |
| NO: 90 | Phenylalanine | ALAKVLRLFEENDVNLTHIESRPSRLKKDEYEFFTHLDKRSLPALT |
| | Hydroxylase | NIIKILRHDIGATVHELSRDKKKDTVPWFPRTIQELDRFANQILSYG |
| | (PAH) | AELDADHPGFKDPVYRARRKQFADIAYNYRHGQPIPRVEYMEEE |
| | | KKTWGTVFKTLKSLYKTHACYEYNHIFPLLEKYCGFHEDNIPQLE |
| | | DVSQFLQTCTGFRLRPVAGLLSSRDFLGGLAFRVFHCTQYIRHGS |
| | | KPMYTPEPDICHELLGHVPLFSDRSFAQFSQEIGLASLGAPDEYIEK |
| | | LATIYWFTVEFGLCKQGDSIKAYGAGLLSSFGELQYCLSEKPKLLP |
| | | LELEKTAIQNYTVTEFQPLYYVAESFNDAKEKVRNFAATIPRPFSV |
| | | RYDPYTQRIEVLDNTQQLKILADSINSEIGILCSALQKIK |
| SEQ ID | N-terminal PAH | MSTAVLENPGLGRKLSDFGQETSYIEDNCNQNGAISLIFSLKEEVG |
| NO: 91 | fragment | ALAKVLRLFEENDVNLTHIESRPSRLKKDEYEFFTHLDKRSLPALT |
| | C237/N-terminal | NIIKILRHDIGATVHELSRDKKKDTVPWFPRTIQELDRFANQILSYG |
| | NpuDnaE intein | AELDADHPGFKDPVYRARRKQFADIAYNYRHGQPIPRVEYMEEE |
| | | KKTWGTVFKTLKSLYKTHACYEYNHIFPLLEKYCGFHEDNIPQLE |
| | | DVSQFLQTCLSYETEILTVEYGLLPIGKIVEKRIECTVYSVDNN |
| | | GNIYTQPVAQWHDRGEQEVFEYCLEDGSLIRATKDHKFMTVD |
| | | GQMLPIDEIFERELDLMRVDNLPN |
| SEQ ID | C-terminal | MIKIATRKYLGKQNVYDIGVERDHNFALKNGFIASNCTGFRLRP |
| NO: 92 | NpuDnaE | VAGLLSSRDFLGGLAFRVFHCTQYIRHGSKPMYTPEPDICHELLGH |
| | intein/C-terminal | VPLFSDRSFAQFSQEIGLASLGAPDEYIEKLATIYWFTVEFGLCKQG |
| | PAH fragment | DSIKAYGAGLLSSFGELQYCLSEKPKLLPLELEKTAIQNYTVTEFQP |
| | C237 | LYYVAESFNDAKEKVRNFAATIPRPFSVRYDPYTQRIEVLDNTQQL |
| | | KILADSINSEIGILCSALQKIK |
| SEQ ID | N-terminal PAH | MSTAVLENPGLGRKLSDFGQETSYIEDNCNQNGAISLIFSLKEEV |
| NO: 93 | fragment C265/N- | GALAKVLRLFEENDVNLTHIESRPSRLKKDEYEFFTHLDKRSLP |
| | terminal NpuDnaE | ALTNIIKILRHDIGATVHELSRDKKKDTVPWFPRTIQELDRFANQI |
| | intein | LSYGAELDADHPGFKDPVYRARRKQFADIAYNYRHGQPIPRVE |
| | | YMEEEKKTWGTVFKTLKSLYKTHACYEYNHIFPLLEKYCGFHE |
| | | DNIPQLEDVSQFLQTCTGFRLRPVAGLLSSRDFLGGLAFRVFHC |
| | | LSYETEILTVEYGLLPIGKIVEKRIECTVYSVDNNGNIYTQPV |
| | | AQWHDRGEQEVFEYCLEDGSLIRATKDHKFMTVDGQMLPI |
| | | DEIFERELDLMRVDNLPN |
| SEQ ID | C-terminal | MIKIATRKYLGKQNVYDIGVERDHNFALKNGFIASNCTQYIRHG |
| NO: 94 | NpuDnaE intein/C- | SKPMYTPEPDICHELLGHVPLFSDRSFAQFSQEIGLASLGAPDEYIE |
| | terminal PAH | KLATIYWFTVEFGLCKQGDSIKAYGAGLLSSFGELQYCLSEKPKLL |
| | fragment C265 | PLELEKTAIQNYTVTEFQPLYYVAESFNDAKEKVRNFAATIPRPFSV |
| | | RYDPYTQRIEVLDNTQQLKILADSINSEIGILCSALQKIK |
| SEQ ID | N-terminal PAH | MSTAVLENPGLGRKLSDFGQETSYIEDNCNQNGAISLIFSLKEEVG |
| NO: 95 | fragment C284/N- | ALAKVLRLFEENDVNLTHIESRPSRLKKDEYEFFTHLDKRSLPALT |
| | terminal NpuDnaE | NIIKILRHDIGATVHELSRDKKKDTVPWFPRTIQELDRFANQILSYG |
| | intein | AELDADHPGFKDPVYRARRKQFADIAYNYRHGQPIPRVEYMEEE |
| | | KKTWGTVFKTLKSLYKTHACYEYNHIFPLLEKYCGFHEDNIPQLE |
| | | DVSQFLQTCTGFRLRPVAGLLSSRDFLGGLAFRVFHCTQYIRHGS |
| | | KPMYTPEPDICLSYETEILTVEYGLLPIGKIVEKRIECTVYSVDN |
| | | NGNIYTQPVAQWHDRGEQEVFEYCLEDGSLIRATKDHKFMTV |
| | | DGQMLPIDEIFERELDLMRVDNLPN |
| SEQ ID | C-terminal | MIKIATRKYLGKQNVYDIGVERDHNFALKNGFIASNCHELLGH |
| NO: 96 | NpuDnaE intein/C- | VPLFSDRSFAQFSQEIGLASLGAPDEYIEKLATIYWFTVEFGLCKQ |
| | terminal PAH | GDSIKAYGAGLLSSFGELQYCLSEKPKLLPLELEKTAIQNYTVTEF |
| | fragment C284 | QPLYYVAESFNDAKEKVRNFAATIPRPFSVRYDPYTQRIEVLDNT |
| | | QQLKILADSINSEIGILCSALQKIK |
| SEQ ID | N-terminal PAH | MSTAVLENPGLGRKLSDFGQETSYIEDNCNQNGAISLIFSLKEEVG |
| NO: 97 | fragment C334/N- | ALAKVLRLFEENDVNLTHIESRPSRLKKDEYEFFTHLDKRSLPALT |
| | terminal NpuDnaE | NIIKILRHDIGATVHELSRDKKKDTVPWFPRTIQELDRFANQILSYG |
| | intein | AELDADHPGFKDPVYRARRKQFADIAYNYRHGQPIPRVEYMEEE |
| | | KKTWGTVFKTLKSLYKTHACYEYNHIFPLLEKYCGFHEDNIPQLE |
| | | DVSQFLQTCTGFRLRPVAGLLSSRDFLGGLAFRVFHCTQYIRHGS |
| | | KPMYTPEPDICHELLGHVPLFSDRSFAQFSQEIGLASLGAPDEYIEK |
| | | LATIYWFTVEFGLCLSYETEILTVEYGLLPIGKIVEKRIECTVYS |
| | | VDNNGNIYTQPVAQWHDRGEQEVFEYCLEDGSLIRATKDHKF |
| | | MTVDGQMLPIDEIFERELDLMRVDNLPN |
| SEQ ID | C-terminal | MIKIATRKYLGKQNVYDIGVERDHNFALKNGFIASNCKQGDSI |
| NO: 98 | NpuDnaE intein/C- | KAYGAGLLSSFGELQYCLSEKPKLLPLELEKTAIQNYTVTEFQPLY |
| | terminal PAH | YVAESFNDAKEKVRNFAATIPRPFSVRYDPYTQRIEVLDNTQQLKI |
| | fragment C334 | LADSINSEIGILCSALQKIK |
| SEQ ID | GTP-CH1 | MEKGPVRAPAEKPRGARCSNGFPERDPPRPGPSRPAEKPPRPEAKS |
| NO: 99 | | AQPADGWKGERPRSEEDNELNLPNLAAAYSSILSSLGENPQRQGL |
| | | LKTPWRAASAMQFFTKGYQETISDVLNDAIFDEDHDEMVIVKDID |
| | | MFSMCEHHLVPFVGKVHIGYLPNKQVLGLSKLARIVEIYSRRLQV |
| | | QERLTKQIAVAITEALRPAGVGVVVEATHMCMVMRGVQKMNSK |
| | | TVTSTMLGVFREDPKTREEFLTLIRS |
| SEQ ID | P2A | GSGATNFSLLKQAGDVEENPGP |
| NO: 100 | | |
| SEQ ID | Full-length PAH-P2A- | MSTAVLENPGLGRKLSDFGQETSYIEDNCNQNGAISLIFSLKEEVGA |
| NO: 101 | (GTP-CH1) | LAKVLRLFEENDVNLTHIESRPSRLKKDEYEFFTHLDKRSLPALTNII |
| | | KILRHDIGATVHELSRDKKKDTVPWFPRTIQELDRFANQILSYGAEL |
| | | DADHPGFKDPVYRARRKQFADIAYNYRHGQPIPRVEYMEEEKKT |
| | | WGTVFKTLKSLYKTHACYEYNHIFPLLEKYCGFHEDNIPQLEDVSQ |
| | | FLQTCTGFRLRPVAGLLSSRDFLGGLAFRVFHCTQYIRHGSKPMYT |
| | | PEPDICHELLGHVPLFSDRSFAQFSQEIGLASLGAPDEYIEKLATIYW |
| | | FTVEFGLCKQGDSIKAYGAGLLSSFGELQYCLSEKPKLLPLELEKTA |
| | | IQNYTVTEFQPLYYVAESFNDAKEKVRNFAATIPRPFSVRYDPYTQ |
| | | RIEVLDNTQQLKILADSINSEIGILCSALQKIKGSGATNFSLLKQAGD |
| | | VEENPGPMEKGPVRAPAEKPRGARCSNGFPERDPPRPGPSRPAE |
| | | KPPRPEAKSAQPADGWKGERPRSEEDNELNLPNLAAAYSSILSS |
| | | LGENPQRQGLLKTPWRAASAMQFFTKGYQETISDVLNDAIFDE |
| | | DHDEMVIVKDIDMFSMCEHHLVPFVGKVHIGYLPNKQVLGLS |
| | | KLARIVEIYSRRLQVQERLTKQIAVAITEALRPAGVGVVVEATH |
| | | MCMVMRGVQKMNSKTVTSTMLGVFREDPKTREEFLTLIRS |
| SEQ ID | N-terminal PAH | MSTAVLENPGLGRKLSDFGQETSYIEDNCNQNGAISLIFSLKEEVGA |
| NO: 102 | fragment C237/N- | LAKVLRLFEENDVNLTHIESRPSRLKKDEYEFFTHLDKRSLPALTNII |
| | terminal NpuDnaE | KILRHDIGATVHELSRDKKKDTVPWFPRTIQELDRFANQILSYGAEL |
| | intein-P2A- | DADHPGFKDPVYRARRKQFADIAYNYRHGQPIPRVEYMEEEKKT |
| | (GTP-CH1) | WGTVFKTLKSLYKTHACYEYNHIFPLLEKYCGFHEDNIPQLEDVSQ |
| | | FLQTCLSYETEILTVEYGLLPIGKIVEKRIECTVYSVDNNGNIYT |
| | | QPVAQWHDRGEQEVFEYCLEDGSLIRATKDHKFMTVDGQML |
| | | PIDEIFERELDLMRVDNLPNGSGATNFSLLKQAGDVEENPGPMEK |
| | | GPVRAPAEKPRGARCSNGFPERDPPRPGPSRPAEKPPRPEAKSAQP |
| | | ADGWKGERPRSEEDNELNLPNLAAAYSSILSSLGENPQRQGLLKTP |
| | | WRAASAMQFFTKGYQETISDVLNDAIFDEDHDEMVIVKDIDMFSM |
| | | CEHHLVPFVGKVHIGYLPNKQVLGLSKLARIVEIYSRRLQVQERLT |
| | | KQIAVAITEALRPAGVGVVVEATHMCMVMRGVQKMNSKTVTSTM |
| | | LGVFREDPKTREEFLTLIRS |
| SEQ ID | C-terminal | MIKIATRKYLGKQNVYDIGVERDHNFALKNGFIASNCTGFRLRP |
| NO: 103 | NpuDnaE intein/C- | VAGLLSSRDFLGGLAFRVFHCTQYIRHGSKPMYTPEPDICHELLGH |
| | terminal PAH | VPLFSDRSFAQFSQEIGLASLGAPDEYIEKLATIYWFTVEFGLCKQG |
| | fragment C237-P2A- | DSIKAYGAGLLSSFGELQYCLSEKPKLLPLELEKTAIQNYTVTEFQP |
| | (GTP-CH1) | LYYVAESFNDAKEKVRNFAATIPRPFSVRYDPYTQRIEVLDNTQQL |
| | | KILADSINSEIGILCSALQKIKGSGATNVSLLKQAGDVEENPGPMEK |
| | | GPVRAPAEKPRGARCSNGFPERDPPRPGPSRPAEKPPRPEAKSAQP |
| | | ADGWKGERPRSEEDNELNLPNLAAAYSSILSSLGENPQRQGLLKTP |
| | | WRAASAMQFFTKGYQETISDVLNDAIFDEDHDEMVIVKDIDMFSM |
| | | CEHHLVPFVGKVHIGYLPNKQVLGLSKLARIVEIYSRRLQVQERLT |
| | | KQIAVAITEALRPAGVGVVVEATHMCMVMRGVQKMNSKTVTSTM |
| | | LGVFREDPKTREEFLTLIRS |
| SEQ ID | N-terminal PAH | MSTAVLENPGLGRKLSDFGQETSYIEDNCNQNGAISLIFSLKEEVGA |
| NO: 104 | fragment C265/N- | LAKVLRLFEENDVNLTHIESRPSRLKKDEYEFFTHLDKRSLPALTNII |
| | terminal NpuDnaE | KILRHDIGATVHELSRDKKKDTVPWFPRTIQELDRFANQILSYGAEL |
| | intein-P2A- | DADHPGFKDPVYRARRKQFADIAYNYRHGQPIPRVEYMEEEKKT |
| | (GTP-CH1) | WGTVFKTLKSLYKTHACYEYNHIFPLLEKYCGFHEDNIPQLEDVSQ |
| | | FLQTCTGFRLRPVAGLLSSRDFLGGLAFRVFHCLSYETEILTVEYG |
| | | LLPIGKIVEKRIECTVYSVDNNGNIYTQPVAQWHDRGEQEVFE |
| | | YCLEDGSLIRATKDHKFMTVDGQMLPIDEIFERELDLMRVDNL |
| | | PNGSGATNFSLLKQAGDVEENPGPMEKGPVRAPAEKPRGARCSNG |
| | | FPERDPPRPGPSRPAEKPPRPEAKSAQPADGWKGERPRSEEDNEL |
| | | NLPNLAAAYSSILSSLGENPQRQGLLKTPWRAASAMQFFTKGYQET |
| | | ISDVLNDAIFDEDHDEMVIVKDIDMFSMCEHHLVPFVGKVHIGYL |
| | | PNKQVLGLSKLARIVEIYSRRLQVQERLTKQIAVAITEALRPAGVGV |
| | | VVEATHMCMVMRGVQKMNSKTVTSTMLGVFREDPKTREEFLTLI |
| | | RS |
| SEQ ID | C-terminal | MIKIATRKYLGKQNVYDIGVERDHNFALKNGFIASNCTQYIRHG |
| NO: 105 | NpuDnaE intein/C- | SKPMYTPEPDICHELLGHVPLFSDRSFAQFSQEIGLASLGAPDEYIEK |
| | terminal PAH | LATIYWFTVEFGLCKQGDSIKAYGAGLLSSFGELQYCLSEKPKLLP |
| | fragment C265-P2A- | LELEKTAIQNYTVTEFQPLYYVAESFNDAKEKVRNFAATIPRPFSVR |
| | (GTP-CH1) | YDPYTQRIEVLDNTQQLKILADSINSEIGILCSALQKIKGSGATNFSL |
| | | LKQAGDVEENPGPMEKGPVRAPAEKPRGARCSNGFPERDPPRPGP |
| | | SRPAEKPPRPEAKSAQPADGWKGERPRSEEDNELNLPNLAAAYSSI |
| | | LSSLGENPQRQGLLKTPWRAASAMQFFTKGYQETISDVLNDAIFDE |
| | | DHDEMVIVKDIDMFSMCEHHLVPFVGKVHIGYLPNKQVLGLSKLA |
| | | RIVEIYSRRLQVQERLTKQIAVAITEALRPAGVGVVVEATHMCMVM |
| | | RGVQKMNSKTVTSTMLGVFREDPKTREEFLTLIRS |
| SEQ ID | N-terminal PAH | MSTAVLENPGLGRKLSDFGQETSYIEDNCNQNGAISLIFSLKEEVGA |
| NO: 106 | fragment C284/N- | LAKVLRLFEENDVNLTHIESRPSRLKKDEYEFFTHLDKRSLPALTNII |
| | terminal NpuDnaE | KILRHDIGATVHELSRDKKKDTVPWFPRTIQELDRFANQILSYGAEL |
| | intein-P2A- | DADHPGFKDPVYRARRKQFADIAYNYRHGQPIPRVEYMEEEKKT |
| | (GTP-CH1) | WGTVFKTLKSLYKTHACYEYNHIFPLLEKYCGFHEDNIPQLEDVSQ |
| | | FLQTCTGFRLRPVAGLLSSRDFLGGLAFRVFHCTQYIRHGSKPMYT |
| | | PEPDICLSYETEILTVEYGLLPIGKIVEKRIECTVYSVDNNGNIYT |
| | | QPVAQWHDRGEQEVFEYCLEDGSLIRATKDHKFMTVDGQML |
| | | PIDEIFERELDLMRVDNLPNGSGATNFSLLKQAGDVEENPGPMEK |
| | | GPVRAPAEKPRGARCSNGFPERDPPRPGPSRPAEKPPRPEAKSAQP |
| | | ADGWKGERPRSEEDNELNLPNLAAAYSSILSSLGENPQRQGLLKTP |
| | | WRAASAMQFFTKGYQETISDVLNDAIFDEDHDEMVIVKDIDMFSM |
| | | CEHHLVPFVGKVHIGYLPNKQVLGLSKLARIVEIYSRRLQVQERLT |
| | | KQIAVAITEALRPAGVGVVVEATHMCMVMRGVQKMNSKTVTSTM |
| | | LGVFREDPKTREEFLTLIRS |
| SEQ ID | C-terminal | MIKIATRKYLGKQNVYDIGVERDHNFALKNGFIASNCHELLGHV |
| NO: 107 | NpuDnaE intein/C- | PLFSDRSFAQFSQEIGLASLGAPDEYIEKLATIYWFTVEFGLCKQGD |
| | terminal PAH | SIKAYGAGLLSSFGELQYCLSEKPKLLPLELEKTAIQNYTVTEFQPL |
| | fragment C284-P2A- | YYVAESFNDAKEKVRNFAATIPRPFSVRYDPYTQRIEVLDNTQQLK |
| | (GTP-CH1) | ILADSINSEIGILCSALQKIKGSGATNFSLLKQAGDVEENPGPMEKG |
| | | PVRAPAEKPRGARCSNGFPERDPPRPGPSRPAEKPPRPEAKSAQPA |
| | | DGWKGERPRSEEDNELNLPNLAAAYSSILSSLGENPQRQGLLKTP |
| | | WRAASAMQFFTKGYQETISDVLNDAIFDEDHDEMVIVKDIDMFSM |
| | | CEHHLVPFVGKVHIGYLPNKQVLGLSKLARIVEIYSRRLQVQERLT |
| | | KQIAVAITEALRPAGVGVVVEATHMCMVMRGVQKMNSKTVTSTM |
| | | LGVFREDPKTREEFLTLIRS |
| SEQ ID | N-terminal PAH | MSTAVLENPGLGRKLSDFGQETSYIEDNCNQNGAISLIFSLKEEVGA |
| NO: 108 | fragment C334/N- | LAKVLRLFEENDVNLTHIESRPSRLKKDEYEFFTHLDKRSLPALTNII |
| | terminal NpuDnaE | KILRHDIGATVHELSRDKKKDTVPWFPRTIQELDRFANQILSYGAEL |
| | intein-P2A- | DADHPGFKDPVYRARRKQFADIAYNYRHGQPIPRVEYMEEEKKT |
| | (GTP-CH1) | WGTVFKTLKSLYKTHACYEYNHIFPLLEKYCGFHEDNIPQLEDVSQ |
| | | FLQTCTGFRLRPVAGLLSSRDFLGGLAFRVFHCTQYIRHGSKPMYT |
| | | PEPDICHELLGHVPLFSDRSFAQFSQEIGLASLGAPDEYIEKLATIYW |
| | | FTVEFGLCLSYETEILTVEYGLLPIGKIVEKRIECTVYSVDNNGNI |
| | | YTQPVAQWHDRGEQEVFEYCLEDGSLIRATKDHKFMTVDGQ |
| | | MLPIDEIFERELDLMRVDNLPNGSGATNFSLLKQAGDVEENPGP |
| | | MEKGPVRAPAEKPRGARCSNGFPERDPPRPGPSRPAEKPPRPEAK |
| | | SAQPADGWKGERPRSEEDNELNLPNLAAAYSSILSSLGENPQRQGL |
| | | LKTPWRAASAMQFFTKGYQETISDVLNDAIFDEDHDEMVIVKDID |
| | | MFSMCEHHLVPFVGKVHIGYLPNKQVLGLSKLARIVEIYSRRLQVQ |
| | | ERLTKQIAVAITEALRPAGVGVVVEATHMCMVMRGVQKMNSKTV |
| | | TSTMLGVFREDPKTREEFLTLIRS |
| SEQ ID | C-terminal | MIKIATRKYLGKQNVYDIGVERDHNFALKNGFIASNCKQGDSIK |
| NO: 109 | NpuDnaE intein/C- | AYGAGLLSSFGELQYCLSEKPKLLPLELEKTAIQNYTVTEFQPLYY |
| | terminal PAH | VAESFNDAKEKVRNFAATIPRPFSVRYDPYTQRIEVLDNTQQLKIL |
| | fragment C334-P2A- | ADSINSEIGILCSALQKIKGSGATNFSLLKQAGDVEENPGPMEKGPV |
| | (GTP-CH1) | RAPAEKPRGARCSNGFPERDPPRPGPSRPAEKPPRPEAKSAQPADG |
| | | WKGERPRSEEDNELNLPNLAAAYSSILSSLGENPQRQGLLKTPWRA |
| | | ASAMQFFTKGYQETISDVLNDAIFDEDHDEMVIVKDIDMFSMCEH |
| | | HLVPFVGKVHIGYLPNKQVLGLSKLARIVEIYSRRLQVQERLTKQIA |
| | | VAITEALRPAGVGVVVEATHMCMVMRGVQKMNSKTVTSTMLGVF |
| | | REDPKTREEFLTLIRS |
| SEQ ID | EGFP | MVSKGEELFTGVVPILVELDGDVNGHKFSVSGEGEGDATYGKLTL |
| NO: 110 | | KFICTTGKLPVPWPTLVTTLTYGVQCFSRYPDHMKQHDFFKSAMP |
| | | EGYVQERTIFFKDDGNYKTRAEVKFEGDTLVNRIELKGIDFKEDGN |
| | | ILGHKLEYNYNSHNVYIMADKQKNGIKVNFKIRHNIEDGSVQLAD |
| | | HYQQNTPIGDGPVLLPDNHYLSTQSALSKDPNEKRDHMVLLEFVT |
| | | AAGITLGMDELYKYSDLELK |
| SEQ ID | mCherry | MVSKGEEDNMAIIKEFMRFKVHMEGSVNGHEFEIEGEGEGRPYEG |
| NO: 111 | | TQTAKLKVTKGGPLPFAWDILSPQFMYGSKAYVKHPADIPDYLKL |
| | | SFPEGFKWERVMNFEDGGVVTVTQDSSLQDGEFIYKVKLRGTNFP |
| | | SDGPVMQKKTMGWEASSERMYPEDGALKGEIKQRLKLKDGGHY |
| | | DAEVKTTYKAKKPVQLPGAYNVNIKLDITSHNEDYTIVEQYERAE |
| | | GRHSTGGMDELYK |
| SEQ ID | Full-length glutamine | MTTSASSHLNKGIKQVYMSLPQGEKVQAMYIWIDGTGEGLRCKTR |
| NO: 112 | synthetase (GS) | TLDSEPKCVEELPEWNFDGSSTLQSEGSNSDMYLVPAAMFRDPFR |
| | | KDPNKLVLCEVFKYNRRPAETNLRHTCKRIMDMVSNQHPWFGME |
| | | QEYTLMGTDGHPFGWPSNGFPGPQGPYYCGVGADRAYGRDIVEA |
| | | HYRACLYAGVKIAGTNAEVMPAQWEFQIGPCEGISMGDHLWVAR |
| | | FILHRVCEDFGVIATFDPKPIPGNWNGAGCHTNFSTKAMREENGLK |
| | | YIEEAIEKLSKRHQYHIRAYDPKGGLDNARRLTGFHETSNINDFSAG |
| | | VANRSASIRIPRTVGQEKKGYFEDRRPSANCDPFSVTEALIRTCLLN |
| | | ETGDEPFQYKN |
| SEQ ID | N-terminal GS | MTTSASSHLNKGIKQVYMSLPQGEKVQAMYIWIDGTGEGLRCKTR |
| NO: 113 | fragment C53/N- | TLDSEPKCLSYETEILTVEYGLLPIGKIVEKRIECTVYSVDNNGNI |
| | terminal NpuDnaE | YTQPVAQWHDRGEQEVFEYCLEDGSLIRATKDHKFMTVDGQ |
| | intein | MLPIDEIFERELDLMRVDNLPN |
| SEQ ID | C-terminal | MIKIATRKYLGKQNVYDIGVERDHNFALKNGFIASNCVEELPEW |
| NO: 114 | NpuDnaE intein/C- | NFDGSSTLQSEGSNSDMYLVPAAMFRDPFRKDPNKLVLCEVFKYN |
| | terminal GS fragment | RRPAETNLRHTCKRIMDMVSNQHPWFGMEQEYTLMGTDGHPFG |
| | C53 | WPSNGFPGPQGPYYCGVGADRAYGRDIVEAHYRACLYAGVKIAG |
| | | TNAEVMPAQWEFQIGPCEGISMGDHLWVARFILHRVCEDFGVIATF |
| | | DPKPIPGNWNGAGCHTNFSTKAMREENGLKYIEEAIEKLSKRHQY |
| | | HIRAYDPKGGLDNARRLTGFHETSNINDFSAGVANRSASIRIPRTVG |
| | | QEKKGYFEDRRPSANCDPFSVTEALIRTCLLNETGDEPFQYKN |
| SEQ ID | N-terminal GS | MTTSASSHLNKGIKQVYMSLPQGEKVQAMYIWIDGTGEGLRCKTR |
| NO: 115 | fragment C117/N- | TLDSEPKCVEELPEWNFDGSSTLQSEGSNSDMYLVPAAMFRDPFR |
| | terminal NpuDnaE | KDPNKLVLCEVFKYNRRPAETNLRHTCLSYETEILTVEYGLLPIG |
| | intein | KIVEKRIECTVYSVDNNGNIYTQPVAQWHDRGEQEVFEYCLED |
| | | GSLIRATKDHKFMTVDGQMLPIDEIFERELDLMRVDNLPN |
| SEQ ID | C-terminal | MIKIATRKYLGKQNVYDIGVERDHNFALKNGFIASNCKRIMDM |
| NO: 116 | NpuDnaE intein/C- | VSNQHPWFGMEQEYTLMGTDGHPFGWPSNGFPGPQGPYYCGVGA |
| | terminal GS fragment | DRAYGRDIVEAHYRACLYAGVKIAGTNAEVMPAQWEFQIGPCEGI |
| | C117 | SMGDHLWVARFILHRVCEDFGVIATFDPKPIPGNWNGAGCHTNFS |
| | | TKAMREENGLKYIEEAIEKLSKRHQYHIRAYDPKGGLDNARRLTG |
| | | FHETSNINDFSAGVANRSASIRIPRTVGQEKKGYFEDRRPSANCDPF |
| | | SVTEALIRTCLLNETGDEPFQYKN |
| SEQ ID | N-terminal GS | MTTSASSHLNKGIKQVYMSLPQGEKVQAMYIWIDGTGEGLRCKTR |
| NO: 117 | fragment C183/N- | TLDSEPKCVEELPEWNFDGSSTLQSEGSNSDMYLVPAAMFRDPFR |
| | terminal NpuDnaE | KDPNKLVLCEVFKYNRRPAETNLRHTCKRIMDMVSNQHPWFGME |
| | intein | QEYTLMGTDGHPFGWPSNGFPGPQGPYYCGVGADRAYGRDIVEA |
| | | HYRACLSYETEILTVEYGLLPIGKIVEKRIECTVYSVDNNGNIYT |
| | | QPVAQWHDRGEQEVFEYCLEDGSLIRATKDHKFMTVDGQML |
| | | PIDEIFERELDLMRVDNLPN |
| SEQ ID | C-terminal | MIKIATRKYLGKQNVYDIGVERDHNFALKNGFIASNCLYAGVKI |
| NO: 118 | NpuDnaE intein/C- | AGTNAEVMPAQWEFQIGPCEGISMGDHLWVARFILHRVCEDFGVI |
| | terminal GS fragment | ATFDPKPIPGNWNGAGCHTNFSTKAMREENGLKYIEEAIEKLSKRH |
| | C183 | QYHIRAYDPKGGLDNARRLTGFHETSNINDFSAGVANRSASIRIPRT |
| | | VGQEKKGYFEDRRPSANCDPFSVTEALIRTCLLNETGDEPFQYKN |
| SEQ ID | N-terminal GS | MTTSASSHLNKGIKQVYMSLPQGEKVQAMYIWIDGTGEGLRCKTR |
| NO: 119 | fragment C229/N- | TLDSEPKCVEELPEWNFDGSSTLQSEGSNSDMYLVPAAMFRDPFR |
| | terminal NpuDnaE | KDPNKLVLCEVFKYNRRPAETNLRHTCKRIMDMVSNQHPWFGME |
| | intein | QEYTLMGTDGHPFGWPSNGFPGPQGPYYCGVGADRAYGRDIVEA |
| | | HYRACLYAGVKIAGTNAEVMPAQWEFQIGPCEGISMGDHLWVAR |
| | | FILHRVCLSYETEILTVEYGLLPIGKIVEKRIECTVYSVDNNGNIY |
| | | TQPVAQWHDRGEQEVFEYCLEDGSLIRATKDHKFMTVDGQM |
| | | LPIDEIFERELDLMRVDNLPN |
| SEQ ID | C-terminal | MIKIATRKYLGKQNVYDIGVERDHNFALKNGFIASNCEDFGVIA |
| NO: 120 | NpuDnaE intein/C- | TFDPKPIPGNWNGAGCHTNFSTKAMREENGLKYIEEAIEKLSKRHQ |
| | terminal GS fragment | YHIRAYDPKGGLDNARRLTGFHETSNINDFSAGVANRSASIRIPRTV |
| | C229 | GQEKKGYFEDRRPSANCDPFSVTEALIRTCLLNETGDEPFQYKN |
| SEQ ID | N-terminal GS | MTTSASSHLNKGIKQVYMSLPQGEKVQAMYIWIDGTGEGLRCKTR |
| NO: 121 | fragment C252/N- | TLDSEPKCVEELPEWNFDGSSTLQSEGSNSDMYLVPAAMFRDPFR |
| | terminal NpuDnaE | KDPNKLVLCEVFKYNRRPAETNLRHTCKRIMDMVSNQHPWFGME |
| | intein | QEYTLMGTDGHPFGWPSNGFPGPQGPYYCGVGADRAYGRDIVEA |
| | | HYRACLYAGVKIAGTNAEVMPAQWEFQIGPCEGISMGDHLWVAR |
| | | FILHRVCEDFGVIATFDPKPIPGNWNGAGCLSYETEILTVEYGLLPI |
| | | GKIVEKRIECTVYSVDNNGNIYTQPVAQWHDRGEQEVFEYCLE |
| | | DGSLIRATKDHKFMTVDGQMLPIDEIFERELDLMRVDNLPN |
| SEQ ID | C-terminal | MIKIATRKYLGKQNVYDIGVERDHNFALKNGFIASNCHTNFSTK |
| NO: 122 | NpuDnaE intein/C- | AMREENGLKYIEEAIEKLSKRHQYHIRAYDPKGGLDNARRLTGFH |
| | terminal GS fragment | ETSNINDFSAGVANRSASIRIPRTVGQEKKGYFEDRRPSANCDPFSV |
| | C252 | TEALIRTCLLNETGDEPFQYKN |
| SEQ ID | Full-length | MPVAGSELPRRPLPPAAQERDAEPRPPHGELQYLGQIQHILRCGVR |
| NO: 123 | Thymidylate | KDDRTGTGTLSVFGMQARYSLRDEFPLLTTKRVFWKGVLEELLWF |
| | Synthase (TYMS) | IKGSTNAKELSSKGVKIWDANGSRDFLDSLGFSTREEGDLGPVYGF |
| | | QWRHFGAEYRDMESDLPLMALPPCHALCQFYVVNSELSCQLYQR |
| | | SGDMGLGVPFNIASYALLTYMIAHITGLKPGDFIHTLGDAHIYLNHI |
| | | EPLKIQLQREPRPFPKLRILRKVEKIDDFKAEDFQIEGYNPHPTIKME |
| | | MAV |
| SEQ ID | N-terminal TYMS | MPVAGSELPRRPLPPAAQERDAEPRPPHGELQYLGQIQHILRCLSY |
| NO: 124 | fragment C41/N- | ETEILTVEYGLLPIGKIVEKRIECTVYSVDNNGNIYTQPVAQWH |
| | terminal NpuDnaE | DRGEQEVFEYCLEDGSLIRATKDHKFMTVDGQMLPIDEIFERE |
| | intein | LDLMRVDNLPN |
| SEQ ID | C-terminal | MIKIATRKYLGKQNVYDIGVERDHNFALKNGFIASNCGVRKDD |
| NO: 125 | NpuDnaE intein/C- | RTGTGTLSVFGMQARYSLRDEFPLLTTKRVFWKGVLEELLWFIKG |
| | terminal TYMS | STNAKELSSKGVKIWDANGSRDFLDSLGFSTREEGDLGPVYGFQW |
| | fragment C41 | RHFGAEYRDMESDLPLMALPPCHALCQFYVVNSELSCQLYQRSGD |
| | | MGLGVPFNIASYALLTYMIAHITGLKPGDFIHTLGDAHIYLNHIEPL |
| | | KIQLQREPRPFPKLRILRKVEKIDDFKAEDFQIEGYNPHPTIKMEMA |
| | | V |
| SEQ ID | N-terminal TYMS | MPVAGSELPRRPLPPAAQERDAEPRPPHGELQYLGQIQHILRCGVR |
| NO: 126 | fragment C161/N- | KDDRTGTGTLSVFGMQARYSLRDEFPLLTTKRVFWKGVLEELLWF |
| | terminal NpuDnaE | IKGSTNAKELSSKGVKIWDANGSRDFLDSLGFSTREEGDLGPVYGF |
| | intein | QWRHFGAEYRDMESDLPLMALPPCLSYETEILTVEYGLLPIGKIV |
| | | EKRIECTVYSVDNNGNIYTQPVAQWHDRGEQEVFEYCLEDGSL |
| | | IRATKDHKFMTVDGQMLPIDEIFERELDLMRVDNLPN |
| SEQ ID | C-terminal | MIKIATRKYLGKQNVYDIGVERDHNFALKNGFIASNCHALCQFY |
| NO: 127 | NpuDnaE intein/C- | VVNSELSCQLYQRSGDMGLGVPFNIASYALLTYMIAHITGLKPGDF |
| | terminal TYMS | IHTLGDAHIYLNHIEPLKIQLQREPRPFPKLRILRKVEKIDDFKAEDF |
| | fragment C161 | QIEGYNPHPTIKMEMAV |
| SEQ ID | N-terminal TYMS | MPVAGSELPRRPLPPAAQERDAEPRPPHGELQYLGQIQHILRCGVR |
| NO: 128 | fragment C165/N- | KDDRTGTGTLSVFGMQARYSLRDEFPLLTTKRVFWKGVLEELLWF |
| | terminal NpuDnaE | IKGSTNAKELSSKGVKIWDANGSRDFLDSLGFSTREEGDLGPVYGF |
| | intein | QWRHFGAEYRDMESDLPLMALPPCHALCLSYETEILTVEYGLLPI |
| | | GKIVEKRIECTVYSVDNNGNIYTQPVAQWHDRGEQEVFEYCLE |
| | | DGSLIRATKDHKFMTVDGQMLPIDEIFERELDLMRVDNLPN |
| SEQ ID | C-terminal | MIKIATRKYLGKQNVYDIGVERDHNFALKNGFIASNCQFYVVNS |
| NO: 129 | NpuDnaE intein/C- | ELSCQLYQRSGDMGLGVPFNIASYALLTYMIAHITGLKPGDFIHTL |
| | terminal TYMS | GDAHIYLNHIEPLKIQLQREPRPFPKLRILRKVEKIDDFKAEDFQIEG |
| | fragment C165 | YNPHPTIKMEMAV |
| SEQ ID | N-terminal TYMS | MPVAGSELPRRPLPPAAQERDAEPRPPHGELQYLGQIQHILRCGVR |
| NO: 130 | fragment C176/N- | KDDRTGTGTLSVFGMQARYSLRDEFPLLTTKRVFWKGVLEELLWF |
| | terminal NpuDnaE | IKGSTNAKELSSKGVKIWDANGSRDFLDSLGFSTREEGDLGPVYGF |
| | intein | QWRHFGAEYRDMESDLPLMALPPCHALCQFYVVNSELSCLSYET |
| | | EILTVEYGLLPIGKIVEKRIECTVYSVDNNGNIYTQPVAQWHDR |
| | | GEQEVFEYCLEDGSLIRATKDHKFMTVDGQMLPIDEIFERELD |
| | | LMRVDNLPN |
| SEQ ID | C-terminal | MIKIATRKYLGKQNVYDIGVERDHNFALKNGFIASNCQLYQRSG |
| NO: 131 | NpuDnaE intein/C- | DMGLGVPFNIASYALLTYMIAHITGLKPGDFIHTLGDAHIYLNHIEP |
| | terminal TYMS | LKIQLQREPRPFPKLRILRKVEKIDDFKAEDFQIEGYNPHPTIKMEM |
| | fragment C176 | AV |
| SEQ ID | Attenuated EF1alpha | AAGGATCTGCGATCGCTCCGGTGCCCGTCAGTGGGCAGAGCGC |
| NO: 132 | promoter | ACATCGCCCACAGTCCCCGAGAAGTTGGGGGGAGGGGTCGGCA |
| | | ATTGAACGGGTGCCTAGAGAAGGTGGCGCGGGGTAAACTGGGA |
| | | AAGTGATGTCGTGTACTGGCTCCGCCTTTTTCCCGAGGGTGGGG |
| | | GAGAACCGTATGTAAGTGCAGTAGTCGCCGTGAACGTTCTTTTT |
| | | CGCAACGGGTTTGCCGCCAGAACACAGCTGAAGCTTCGAGGGG |
| | | CTCGCATCTCTCCTTCACGCGCCCGCCGCCCTACCTGAGGCCGC |
| | | CATCCACGCCGGTTGAGTCGCGTTCTGCCGCCTCCCGCCTGTGG |
| | | TGCCTCCTGAACTGCGTCCGCCGTCTAGGTAAGTTTAAAGCTCA |
| | | GGTCGAGACCGGGCCTTTGTCCGGCGCTCCCTTGGAGCCTACCT |
| | | AGACTCAGCCGGCTCTCCACGCTTTGCCTGACCCTGCTTGCTCA |
| | | ACTCTACGTCTTTGTTTCGTTTTCTGTTCTGCGCCGTTACAGATC |
| | | CAAGCTGTGACCGGCGCCTAC |
| SEQ ID | EF1 alpha promoter | AAGGATCTGCGATCGCTCCGGTGCCCGTCAGTGGGCAGAGCGC |
| NO: 133 | | ACATCGCCCACAGTCCCCGAGAAGTTGGGGGGAGGGGTCGGCA |
| | | ATTGAACGGGTGCCTAGAGAAGGTGGCGCGGGGTAAACTGGGA |
| | | AAGTGATGTCGTGTACTGGCTCCGCCTTTTTCCCGAGGGTGGGG |
| | | GAGAACCGTATATAAGTGCAGTAGTCGCCGTGAACGTTCTTTTT |
| | | CGCAACGGGTTTGCCGCCAGAACACAGCTGAAGCTTCGAGGGG |
| | | CTCGCATCTCTCCTTCACGCGCCCGCCGCCCTACCTGAGGCCGC |
| | | CATCCACGCCGGTTGAGTCGCGTTCTGCCGCCTCCCGCCTGTGG |
| | | TGCCTCCTGAACTGCGTCCGCCGTCTAGGTAAGTTTAAAGCTCA |
| | | GGTCGAGACCGGGCCTTTGTCCGGCGCTCCCTTGGAGCCTACCT |
| | | AGACTCAGCCGGCTCTCCACGCTTTGCCTGACCCTGCTTGCTCA |
| | | ACTCTACGTCTTTGTTTCGTTTTCTGTTCTGCGCCGTTACAGATC |
| | | CAAGCTGTGACCGGCGCCTAC |
| SEQ ID | CMV promoter | ACTAGTATTATGCCCAGTACATGACCTTATGGGACTTTCCTACTT |
| NO: 134 | | GGCAGTACATCTACGTATTAGTCATCGCTATTACCATGGTGATG |
| | | CGGTTTTGGCAGTACATCAATGGGCGTGGATAGCGGTTTGACTC |
| | | ACGGGGATTTCCAAGTCTCCACCCCATTGACGTCAATGGGAGTT |
| | | TGTTTTGGCACCAAAATCAACGGGACTTTCCAAAATGTCGTAAC |
| | | AACTCCGCCCCATTGACGCAAATGGGCGGTAGGCGTGTACGGT |
| | | GGGAGGTTTATATAAGCAGAGCTCGTTTAGTGAACCGTCAGATC |
| | | GCCTGGAGACGCCATCCACGCTGTTTTGACCTCCATAGAAGA |
| SEQ ID | IRES | GCCCCTCTCCCTCCCCCCCCCCTAACGTTACTGGCCGAAGCCGC |
| NO: 135 | | TTGGAATAAGGCCGGTGTGCGTTTGTCTATATGTTATTTTCCACC |
| | | ATATTGCCGTCTTTTGGCAATGTGAGGGCCCGGAAACCTGGCCC |
| | | TGTCTTCTTGACGAGCATTCCTAGGGGTCTTTCCCCTCTCGCCAA |
| | | AGGAATGCAAGGTCTGTTGAATGTCGTGAAGGAAGCAGTTCCTC |
| | | TGGAAGCTTCTTGAAGACAAACAACGTCTGTAGCGACCCTTTGC |
| | | AGGCAGCGGAACCCCCCACCTGGCGACAGGTGCCTCTGCGGCC |
| | | AAAAGCCACGTGTATAAGATACACCTGCAAAGGCGGCACAACC |
| | | CCAGTGCCACGTTGTGAGTTGGATAGTTGTGGAAAGAGTCAAAT |
| | | GGCTCTCCTCAAGCGTATTCAACAAGGGGCTGAAGGATGCCCA |
| | | GAAGGTACCCCATTGTATGGGATCTGATCTGGGGCCTCGGTGCA |
| | | CATGCTTTACATGTGTTTAGTCGAGGTTAAAAAAACGTCTAGGC |
| | | CCCCCGAACCACGGGGACGTGGTTTTCCTTTGAAAAACACGATG |
| | | ATAATATGGCCACAACC |
| SEQ ID | Rep2BFP- | GGAGGGGTGGAGTCGTGACGTGAATTACGTCATAGGGTTAGGG |
| NO: 136 | CODE/Cap5 | AGGTCCTGTATTAGAGGTCACGTGAGTGTTTTGCGACATTTTGC |
| | construct | GACACCATGTGGTCACGCTGGGTATTTAAGCCCGAGTGAGCACG |
| | | CAGGGTCTCCATTTTGAAGCGGGAGGTTTGAACGCGCAGCCGCC |
| | | ATGCCGGGGTTTTACGAGATTGTGATTAAGGTCCCCAGCGACCT |
| | | TGACGAGCATCTGCCCGGCATTTCTGACAGCTTTGTGAACTGGG |
| | | TGGCCGAGAAGGAATGGGAGTTGCCGCCAGATTCTGACATGGA |
| | | TCTGAATCTGATTGAGCAGGCACCCCTGACCGTGGCCGAGAAGC |
| | | TGCAGCGCGACTTTCTGACGGAATGGCGCCGTGTGAGTAAGGCC |
| | | CCGGAGGCCCTTTTCTTTGTGCAATTTGAGAAGGGAGAGAGCTA |
| | | CTTCCACATGCACGTGCTCGTGGAAACCACCGGGGTGAAATCCA |
| | | TGGTTTTGGGACGTTTCCTGAGTCAGATTCGCGAAAAACTGATT |
| | | CAGAGAATTTACCGCGGGATCGAGCCGACTTTGCCAAACTGGTT |
| | | CGCGGTCACAAAGACCAGAAATGGCGCCGGAGGCGGGAACAA |
| | | GGTGGTGGATGAGTGCTACATCCCCAATTACTTGCTCCCCAAAA |
| | | CCCAGCCTGAGCTCCAGTGGGCGTGGACTAATATGGAACAGTAT |
| | | TTAAGCGCCTGTTTGAATCTCACGGAGCGTAAACGGTTGGTGGC |
| | | GCAGCATCTGACGCACGTGTCGCAGACGCAGGAGCAGAACAAA |
| | | GAGAATCAGAATCCCAATTCTGATGCGCCGGTGATCAGATCAA |
| | | AAACTTCAGCCAGGTACATGGAGCTGGTCGGGTGGCTCGTGGA |
| | | CAAGGTGAGTTTGGGGACCCTTGATTGTTCTTTCTTTTTCGCTAT |
| | | TGTAAAATTCATGTTATATGGAGGGGGCAAAGTTTTCAGGGTGT |
| | | TGTTTAGAATGGGAAGATGTCCCTTGTATCACCATGGACCCTCA |
| | | TGATAATTTTGTTTCTTTCACTTTCTACTCTGTTGACAACCGTTGT |
| | | CTCCTCTTATTTTCTTTTCATTTTCTGTAACTTTTTCGTTAAACTT |
| | | TAGCTTGCATTTGTAACGAATTTTTAAATTCACTTTTGTTTATTT |
| | | GTCAGATTGTAAGTACTTTCTCTAATCACTTTTTTTTCAAGGCAA |
| | | TCAGGGTATATTATATTGTACTTCAGCACAGTTTTAGAGAACAT |
| | | AACTTCGTATAAAGTATACTATACGAAGTTATCGGGCCCCTCTG |
| | | CTAACCATGTTCATGCCTTCTTCTTTTTCCTACAGATGTCAGAAC |
| | | TCATTAAAGAGAATATGCACATGAAGCTGTATATGGAAGGTACT |
| | | GTAGACAACCACCATTTCAAATGCACGTCCGAAGGTGAGGGGA |
| | | AGCCATACGAGGGTACCCAAACTATGCGCATCAAAGTGGTTGA |
| | | GGGTGGCCCCCTGCCATTCGCATTCGACATCCTGGCAACTAGCT |
| | | TTCTTTACGGTTCCAAGACATTCATAAATCATACCCAGGGTATT |
| | | CCCGATTTCTTCAAACAATCCTTCCCGGAAGGGTTTACTTGGGA |
| | | GCGGGTCACGACATATGAAGACGGGGGTGTTCTTACAGCCACA |
| | | CAGGATACGAGTTTGCAAGACGGTTGTCTTATCTATAACGTGAA |
| | | GATTCGGGGTGTGAATTTCACATCCAATGGCCCGGTGATGCAGA |
| | | AAAAAACACTGGGCTGGGAAGCATTTACGGAGACGTTGTATCC |
| | | CGCCGATGGAGGTCTCGAGGGCCGAAACGATATGGCCCTCAAG |
| | | TTGGTAGGTGGTTCTCACCTTATAGCAAACATTAAGACCACGTA |
| | | TCGATCAAAAAAACCCGCTAAGAATCTGAAAATGCCAGGCGTG |
| | | TATTATGTTGATTACAGACTGGAGCGAATAAAAGAGGCTAACA |
| | | ATGAGACCTACGTCGAACAGCATGAAGTCGCTGTAGCTAGATAT |
| | | TGCGACCTCCCGTCAAAGTTGGGCCATAAATTGAATTAACCTCA |
| | | GGTGCAGGCTGCCTATCAGAAGGTGGTGGCTGGTGTGGCCAAT |
| | | GCCCTGGCTCACAAATACCACTGAGATCTTTTTCCCTCTGCCAA |
| | | AAATTATGGGGACATCATGAAGCCCCTTGAGCATCTGACTTCTG |
| | | GCTAATAAAGGAAATTTATTTTCATTGCAATAGTGTGTTGGAAT |
| | | TTTTTGTGTCTCTCACTCGGAAGGACATATGGGAGGGCAAATCA |
| | | TTTAAAACATCAGAATGAGTATTTGGTTTAGAGTTTGGCAACAT |
| | | ATGCCCATATGCTGGCTGCCATGAACAAAGGTTGGCTATAAAGA |
| | | GGTCATCAGTATATGAAACAGCCCCCTGCTGTCCATTCCTTATTC |
| | | CATAGAAAAGCCTTGACTTGAGGTTAGATTTTTTTTATATTTTGT |
| | | TTTGTGTTATTTTTTTCTTTAACATCCCTAAAATTTTCCTTACATG |
| | | TTTTACTAGCCAGATTTTTCCTCCTCTCCTGACTACTCCCAGTCA |
| | | TAGCTGTCCCTCTTCTCTTATGGAGATCATAACTTCGTATAAAGT |
| | | ATACTATACGAAGTTATAATTGTTATAATTAAATGATAAGGTAG |
| | | AATATTTCTGCATATAAATTCTGGCTGGCGTGGAAATATTCTTAT |
| | | TGGTAGAAACAACTACACCCTGGTCATCATCCTGCCTTTCTCTTT |
| | | ATGGTTACAATGATATACACTGTTTGAGATGAGGATAAAATACT |
| | | CTGAGTCCAAACCGGGCCCCTCTGCTAACCATGTTCATGCCTTC |
| | | TTCTTTTTCCTACAGGGGATTACCTCGGAGAAGCAGTGGATCCA |
| | | GGAGGACCAGGCCTCATACATCTCCTTCAATGCGGCCTCCAACT |
| | | CGCGGTCCCAAATCAAGGCTGCCTTGGACAATGCGGGAAAGAT |
| | | TATGAGCCTGACTAAAACCGCCCCCGACTACCTGGTGGGCCAGC |
| | | AGCCCGTGGAGGACATTTCCAGCAATCGGATTTATAAAATTTTG |
| | | GAACTAAACGGGTACGATCCCCAATATGCGGCTTCCGTCTTTCT |
| | | GGGATGGGCCACGAAAAAGTTCGGCAAGAGGAACACCATCTGG |
| | | CTGTTTGGGCCTGCAACTACCGGGAAGACCAACATCGCGGAGG |
| | | CCATAGCCCACACTGTGCCCTTCTACGGGTGCGTAAACTGGACC |
| | | AATGAGAACTTTCCCTTCAACGACTGTGTCGACAAGATGGTGAT |
| | | CTGGTGGGAGGAGGGGAAGATGACCGCCAAGGTCGTGGAGTCG |
| | | GCCAAAGCCATTCTCGGAGGAAGCAAGGTGCGCGTGGACCAGA |
| | | AATGCAAGTCCTCGGCCCAGATAGACCCGACTCCCGTGATCGTC |
| | | ACCTCCAACACCAACATGTGCGCCGTGATTGACGGGAACTCAAC |
| | | GACCTTCGAACACCAGCAGCCGTTGCAAGACCGGATGTTCAAAT |
| | | TTGAACTCACCCGCCGTCTGGATCATGACTTTGGGAAGGTCACC |
| | | AAGCAGGAAGTCAAAGACTTTTTCCGGTGGGCAAAGGATCACG |
| | | TGGTTGAGGTGGAGCATGAATTCTACGTCAAAAAGGGTGGAGC |
| | | CAAGAAAAGACCCGCCCCCAGTGACGCAGATATAAGTGAGCCC |
| | | AAACGGGTGCGCGAGTCAGTTGCGCAGCCATCGACGTCAGACG |
| | | CGGAAGCTTCGATCAACTACGCAGACAGGTACCAAAACAAATG |
| | | TTCTCGTCACGTGGGCATGAATCTGATGCTGTTTCCCTGCAGAC |
| | | AATGCGAGAGAATGAATCAGAATTCAAATATCTGCTTCACTCAC |
| | | GGACAGAAAGACTGTTTAGAGTGCTTTCCCGTGTCAGAATCTCA |
| | | ACCCGTTTCTGTCGTCAAAAAGGCGTATCAGAAACTGTGCTACA |
| | | TTCATCATATCATGGGAAAGGTGCCAGACGCTTGCACTGCCTGC |
| | | GATCTGGTCAATGTGGATTTGGATGACTGCATCTTTGAACAATA |
| | | AATGATTTGTAAATAAATTTAGTAGTCATGTCTTTTGTTGATCAC |
| | | CCTCCAGATTGGTTGGAAGAAGTTGGTGAAGGTCTTCGCGAGTT |
| | | TTTGGGCCTTGAAGCGGGCCCACCGAAACCAAAACCCAATCAG |
| | | CAGCATCAAGATCAAGCCCGTGGTCTTGTGCTGCCTGGTTATAA |
| | | CTATCTCGGACCCGGAAACGGTCTCGATCGAGGAGAGCCTGTCA |
| | | ACAGGGCAGACGAGGTCGCGCGAGAGCACGACATCTCGTACAA |
| | | CGAGCAGCTTGAGGCGGGAGACAACCCCTACCTCAAGTACAAC |
| | | CACGCGGACGCCGAGTTTCAGGAGAAGCTCGCCGACGACACAT |
| | | CCTTCGGGGGAAACCTCGGAAAGGCAGTCTTTCAGGCCAAGAA |
| | | AAGGGTTCTCGAACCTTTTGGCCTGGTTGAAGAGGGTGCTAAGA |
| | | CGGCCCCTACCGGAAAGCGGATAGACGACCACTTTCCAAAAAG |
| | | AAAGAAGGCTCGGACCGAAGAGGACTCCAAGCCTTCCACCTCG |
| | | TCAGACGCCGAAGCTGGACCCAGCGGATCCCAGCAGCTGCAAA |
| | | TCCCAGCCCAACCAGCCTCAAGTTTGGGAGCTGATACAATGTCT |
| | | GCGGGAGGTGGCGGCCCATTGGGCGACAATAACCAAGGTGCCG |
| | | ATGGAGTGGGCAATGCCTCGGGAGATTGGCATTGCGATTCCACG |
| | | TGGATGGGGGACAGAGTCGTCACCAAGTCCACCCGAACCTGGG |
| | | TGCTGCCCAGCTACAACAACCACCAGTACCGAGAGATCAAAAG |
| | | CGGCTCCGTCGACGGAAGCAACGCCAACGCCTACTTTGGATACA |
| | | GCACCCCCTGGGGGTACTTTGACTTTAACCGCTTCCACAGCCAC |
| | | TGGAGCCCCCGAGACTGGCAAAGACTCATCAACAACTACTGGG |
| | | GCTTCAGACCCCGGTCCCTCAGAGTCAAAATCTTCAACATTCAA |
| | | GTCAAAGAGGTCACGGTGCAGGACTCCACCACCACCATCGCCA |
| | | ACAACCTCACCTCCACCGTCCAAGTGTTTACGGACGACGACTAC |
| | | CAGCTGCCCTACGTCGTCGGCAACGGGACCGAGGGATGCCTGC |
| | | CGGCCTTCCCTCCGCAGGTCTTTACGCTGCCGCAGTACGGTTAC |
| | | GCGACGCTGAACCGCGACAACACAGAAAATCCCACCGAGAGGA |
| | | GCAGCTTCTTCTGCCTAGAGTACTTTCCCAGCAAGATGCTGAGA |
| | | ACGGGCAACAACTTTGAGTTTACCTACAACTTTGAGGAGGTGCC |
| | | CTTCCACTCCAGCTTCGCTCCCAGTCAGAACCTGTTCAAGCTGG |
| | | CCAACCCGCTGGTGGACCAGTACTTGTACCGCTTCGTGAGCACA |
| | | AATAACACTGGCGGAGTCCAGTTCAACAAGAACCTGGCCGGGA |
| | | GATACGCCAACACCTACAAAAACTGGTTCCCGGGGCCCATGGG |
| | | CCGAACCCAGGGCTGGAACCTGGGCTCCGGGGTCAACCGCGCC |
| | | AGTGTCAGCGCCTTCGCCACGACCAATAGGATGGAGCTCGAGG |
| | | GCGCGAGTTACCAGGTGCCCCCGCAGCCGAACGGCATGACCAA |
| | | CAACCTCCAGGGCAGCAACACCTATGCCCTGGAGAACACTATG |
| | | ATCTTCAACAGCCAGCCGGCGAACCCGGGCACCACCGCCACGT |
| | | ACCTCGAGGGCAACATGCTCATCACCAGCGAGAGCGAGACGCA |
| | | GCCGGTGAACCGCGTGGCGTACAACGTCGGCGGGCAGATGGCC |
| | | ACCAACAACCAGAGCTCCACCACTGCCCCCGCGACCGGCACGT |
| | | ACAACCTCCAGGAAATCGTGCCCGGCAGCGTGTGGATGGAGAG |
| | | GGACGTGTACCTCCAAGGACCCATCTGGGCCAAGATCCCAGAG |
| | | ACGGGGGCGCACTTTCACCCCTCTCCGGCCATGGGCGGATTCGG |
| | | ACTCAAACACCCACCGCCCATGATGCTCATCAAGAACACGCCTG |
| | | TGCCCGGAAATATCACCAGCTTCTCGGACGTGCCCGTCAGCAGC |
| | | TTCATCACCCAGTACAGCACCGGGCAGGTCACCGTGGAGATGG |
| | | AGTGGGAGCTCAAGAAGGAAAACTCCAAGAGGTGGAACCCAGA |
| | | GATCCAGTACACAAACAACTACAACGACCCCCAGTTTGTGGACT |
| | | TTGCCCCGGACAGCACCGGGGAATACAGAACCACCAGACCTAT |
| | | CGGAACCCGATACCTTACCCGACCCCTTTAATTGCTTGTTAATC |
| | | AATAAACCGTTTAATTCGTTTCAGTTGAACTTTGGTCTCTGCGTA |
| | | TTTCTTTCTTATCTAGTTTCCATGGCTACGTAGATAAGTAGCATG |
| | | GCGGGTTAATCATTAACTACAGCCCGGGCGTTTAAACAGCGGGC |
| | | GGAGGGGTGGAGTCGTGACGTGAATTACGTCATAGGGTTAGGG |
| | | AGGTCCTGTATTAGAGGTCACGTGAGTGTTTTGCGACATTTTGC |
| | | GACACCATGT |
| SEQ ID | N-terminal Blasticidin | GAAAACATTTAACATTTCTCAACAGGATCTAGAATTAGTAGAAG |
| NO: 137 | fragment/N-terminal | TAGCGACAGAGAAGATTACAATGCTTTATGAGGATAATAAACA |
| | NpuDnaE Intein | TCATGTGGGAGCGGCAATTCGTACGAAAACAGGAGAAATCATT |
| | | TCGGCAGTACATATTGAAGCGTATATAGGACGAGTAACTGTTTG |
| | | TGCAGAAGCCATTGCGATTGGTAGTGCAGTTTCGAATGGACAAA |
| | | AGGATTTTGACACGATTGTAGCTGTTAGACACCCTTATTCTGAC |
| | | GAAGTAGATAGAAGTATTCGAGTGGTAAGTCCTTGTGGTATGTG |
| | | CCTTTCATACGAGACCGAGATCCTGACTGTCGAGTACGGATTGC |
| | | TTCCTATCGGCAAAATCGTGGAGAAGAGGATTGAATGTACCGTC |
| | | TATTCAGTCGATAATAATGGGAACATCTACACACAGCCCGTGGC |
| | | TCAATGGCACGACAGAGGAGAGCAGGAAGTTTTTGAATACTGT |
| | | CTCGAGGACGGATCCCTCATCCGCGCTACTAAAGATCATAAGTT |
| | | TATGACCGTGGACGGCCAGATGCTGCCAATTGACGAAATTTTTG |
| | | AACGAGAGCTGGATCTGATGAGAGTCGACAACCTTCCAAACTG |
| | | A |
| SEQ ID | C-terminal NpuDnaE | ATGATTAAGATCGCTACGCGGAAGTACCTGGGGAAACAGAACG |
| NO: 138 | Intein/C-terminal | TCTACGACATAGGTGTGGAGCGCGATCACAACTTTGCTCTGAAA |
| | Blasticidin fragment | AATGGATTTATCGCCAGCAACTGTAGGGAGTTGATTTCAGACTA |
| | | TGCACCAGATTGTTTTGTGTTAATAGAAATGAATGGCAAGTTAG |
| | | TCAAAACTACGATTGAAGAACTCATTCCACTCAAATATACCCGA |
| | | AATT |
| SEQ ID | GFP AAV (ITR to ITR) | GCTTTTGGCCACTCCCTCTCTGCGCGCTCGCTCGCTCACTGAGGC |
| NO: 139 | | CGGGCGACCAAAGGTCGCCCGACGCCCGGGCTTTGCCCGGGCG |
| | | GCCTCAGTGAGCGAGCGAGCGCGCAGAGAGGGAGTGGCCAACT |
| | | CCATCACTAGGGGTTCCTCTGCAGCCGCGACCGGCCAAGGTTTA |
| | | ATGATAGGCTGCAACGGGATGTTGGGAATATGTTGCACTGGTCC |
| | | GTGAGGGTACCAACTTGTTTATTGCAGCTTATAATGGTTACAAA |
| | | TAAAGCAATAGCATCACAAATTTCACAAATAAAGCATTTTTTTC |
| | | ACTGCATTCTAGTTGTGGTTTGTCCAAACTCATCAATGTATCTTA |
| | | TCATGTCTGACCGGTTCACTTGAGCTCGAGATCTGAGTACTTGT |
| | | ACAGCTCGTCCATGCCGAGAGTGATCCCGGCGGCGGTCACGAA |
| | | CTCCAGCAGGACCATGTGATCGCGCTTCTCGTTGGGGTCTTTGC |
| | | TCAGGGCGGACTGGGTGCTCAGGTAGTGGTTGTCGGGCAGCAG |
| | | CACGGGGCCGTCGCCGATGGGGGTGTTCTGCTGGTAGTGGTCGG |
| | | CGAGCTGCACGCTGCCGTCCTCGATGTTGTGGCGGATCTTGAAG |
| | | TTCACCTTGATGCCGTTCTTCTGCTTGTCGGCCATGATATAGACG |
| | | TTGTGGCTGTTGTAGTTGTACTCCAGCTTGTGCCCCAGGATGTTG |
| | | CCGTCCTCCTTGAAGTCGATGCCCTTCAGCTCGATGCGGTTCAC |
| | | CAGGGTGTCGCCCTCGAACTTCACCTCGGCGCGGGTCTTGTAGT |
| | | TGCCGTCGTCCTTGAAGAAGATGGTGCGCTCCTGGACGTAGCCT |
| | | TCGGGCATGGCGGACTTGAAGAAGTCGTGCTGCTTCATGTGGTC |
| | | GGGGTAGCGGCTGAAGCACTGCACGCCGTAGGTCAGGGTGGTC |
| | | ACGAGGGTGGGCCAGGGCACGGGCAGCTTGCCGGTGGTGCAGA |
| | | TGAACTTCAGGGTCAGCTTGCCGTAGGTGGCATCGCCCTCGCCC |
| | | TCGCCGGACACGCTGAACTTGTGGCCGTTTACGTCGCCGTCCAG |
| | | CTCGACCAGGATGGGCACCACCCCGGTGAACAGCTCCTCGCCCT |
| | | TGCTCACCATGGTGGCGGCTTAAGGGTTCGATCCTCTAGAGTCC |
| | | GGAGGCTGGATCGGTCCCGGTGTCTACTATGGAGGTCAAAACA |
| | | GCGTGGATGGCGTCTCCAGGCGATCTGACGGTTCACTAAACGAG |
| | | CTCTGCTTATATAGACCTCCCACCGTACACGCCTACCGCCCATTT |
| | | GCGTCAATGGGGCGGAGTTGTTACGACATTTTGGAAAGTCCCGT |
| | | TGATTTTGGTGCCAAAACAAACTCCCATTGACGTCAATGGGGTG |
| | | GAGACTTGGAAATCCCCGTGAGTCAAACCGCTATCCACGCCCAT |
| | | TGATGTACTGCCAAAACCGCATCACCATGGTAATAGCGATGACT |
| | | AATACGTAGATGTACTGCCAAGTAGGAAAGTCCCATAAGGTCA |
| | | TGTACTGGGCATAATGCCAGGCGGGCCATTTACCGTCATTGACG |
| | | TCAATAGGGGGCGTACTTGGCATATGATACACTTGATGTACTGC |
| | | CAAGTGGGCAGTTTACCGTAAATACTCCACCCATTGACGTCAAT |
| | | GGAAAGTCCCTATTGGCGTTACTATGGGAACATACGTCATTATT |
| | | GACGTCAATGGGCGGGGGTCGTTGGGCGGTCAGCCAGGCGGGC |
| | | CATTTACCGTAAGTTATGTAACGCGGAACTCCATATATGGGCTA |
| | | TGAACTAATGACCCCGTAATTGATTACTATTAATAACTAGTCAA |
| | | TAATCAATGTCAACGCGTATGGTACCTGCGGAGGATGCCGAGG |
| | | ATAACCTTGTTACTAGCCTCCGCCTGGCCGTTGGACTGTGGATA |
| | | ATATGGCGTAGAGGATCCTCTGCGCGCTCGCTCGCTCACTGAGG |
| | | CCGCCCGGGCAAAGCCCGGGCGTCGGGCGACCTTTGGTCGCCC |
| | | GGCCTCAGTGAGCGAGCGAGCGCGCAGAGAA |
| SEQ ID | N-term NpuDnaE | CLSYETEILTVEYGLLPIGKIVEKRIECTVYSVDNNGNIYTQPVAQW |
| NO: 140 | intein fragment | HDRGEQEVFEYCLEDGSLIRATKDHKFMTVDGQMLPIDEIFERELD |
| | | LMRVDNLPN |
| SEQ ID | C-term NpuDnaE | MIKIATRKYLGKQNVYDIGVERDHNFALKNGFIASN |
| NO: 141 | intein fragment | |
| SEQ ID | Mutant GS R324C | MTTSASSHLNKGIKQVYMSLPQGEKVQAMYIWIDGTGEGLRCK |
| NO: 142 | | TRTLDSEPKCVEELPEWNFDGSSTLQSEGSNSDMYLVPAAMFRD |
| | | PFRKDPNKLVLCEVFKYNRRPAETNLRHTCKRIMDMVSNQHPW |
| | | FGMEQEYTLMGTDGHPFGWPSNGFPGPQGPYYCGVGADRAYG |
| | | RDIVEAHYRACLYAGVKIAGTNAEVMPAQWEFQIGPCEGISMGD |
| | | HLWVARFILHRVCEDFGVIATFDPKPIPGNWNGAGCHTNFSTKA |
| | | MREENGLKYIEEAIEKLSKRHQYHIRAYDPKGGLDNARRLTGFH |
| | | ETSNINDFSAGVANRSASICIPRTVGQEKKGYFEDRRPSANCDPFS |
| | | VTEALIRTCLLNETGDEPFQYKN |
| SEQ ID | Mutant GS R324S | MTTSASSHLNKGIKQVYMSLPQGEKVQAMYIWIDGTGEGLRCK |
| NO: 143 | | TRTLDSEPKCVEELPEWNFDGSSTLQSEGSNSDMYLVPAAMFRD |
| | | PFRKDPNKLVLCEVFKYNRRPAETNLRHTCKRIMDMVSNQHPW |
| | | FGMEQEYTLMGTDGHPFGWPSNGFPGPQGPYYCGVGADRAYG |
| | | RDIVEAHYRACLYAGVKIAGTNAEVMPAQWEFQIGPCEGISMGD |
| | | HLWVARFILHRVCEDFGVIATFDPKPIPGNWNGAGCHTNFSTKA |
| | | MREENGLKYIEEAIEKLSKRHQYHIRAYDPKGGLDNARRLTGFH |
| | | ETSNINDFSAGVANRSASISIPRTVGQEKKGYFEDRRPSANCDPFS |
| | | VTEALIRTCLLNETGDEPFQYKN |
| SEQ ID | Mutant GS R341C | MTTSASSHLNKGIKQVYMSLPQGEKVQAMYIWIDGTGEGLRCK |
| NO: 144 | | TRTLDSEPKCVEELPEWNFDGSSTLQSEGSNSDMYLVPAAMFRD |
| | | PFRKDPNKLVLCEVFKYNRRPAETNLRHTCKRIMDMVSNQHPW |
| | | FGMEQEYTLMGTDGHPFGWPSNGFPGPQGPYYCGVGADRAYG |
| | | RDIVEAHYRACLYAGVKIAGTNAEVMPAQWEFQIGPCEGISMGD |
| | | HLWVARFILHRVCEDFGVIATFDPKPIPGNWNGAGCHTNFSTKA |
| | | MREENGLKYIEEAIEKLSKRHQYHIRAYDPKGGLDNARRLTGFH |
| | | ETSNINDFSAGVANRSASIRIPRTVGQEKKGYFEDRCPSANCDPFS |
| | | VTEALIRTCLLNETGDEPFQYKN |
| SEQ ID | P5 promoter | GGAGGGGTGGAGTCGTGACGTGAATTACGTCATAGGGTTAGG |
| NO: 145 | sequence | GAGGTCCTGTATTAGAGGTCACGTGAGTGTTTTGCGACATTTT |
| | | GCGACACCATGTG |
| SEQ ID | Progranulin payload | CACTCCCTCTCTGCGCGCTCGCTCGCTCACTGAGGCCGGGCGAC |
| NO: 146 | (ITR to ITR) (of | CAAAGGTCGCCCGACGCCCGGGCTTTGCCCGGGCGGCCTCAGTG |
| | SEQ ID NO: 147) | AGCGAGCGAGCGCGCAGAGAGGGAGTGGCCAACTCCATCACTA |
| | | GGGGTTCCTCTGCAGTGAGAGACACAAAAAATTCCAACACACT |
| | | ATTGCAATGAAAATAAATTTCCTTTATTTCACAGCAGCTGTCTC |
| | | AAGGCTGGGTCCCTCAAAGGGGCGTCCCAGCGCGGGGCCTCCC |
| | | TGCGCAAACACTTGGTACCCCTGGCTGCGCAGCGGAAGCCAGC |
| | | AGGACAGCAGTGGCGCCGATCAGCACAACAGACGCCCTGGCGG |
| | | TAGGGACAGCAGGCCCAGCCCTGTCGGTTGTCTCGGCAGCAGGT |
| | | CTGGTTATCATGGCAGAAGTGTCCTTCCCCACACTCCACGTCCTT |
| | | CACACCCACGTGAGGGCTACGGGCCAGGAAGGTGGCAGGCTGG |
| | | GCAGAGACCACTTCCTTCTCGCAGGATCGAGCCTTCACGTTGCA |
| | | GGTGTAGCCAGCCGGGCAGCAGTGCTGGCGATCCTCGCAGCAC |
| | | ACAGCATGGGGCAACTGGCAGCAGGCCCAGCTCCCACCCAGGC |
| | | TCGGGCAGCAGGTCTGCCCCACCGGGCAGCTGGTGTGCTGGTCA |
| | | CAGCCGATGTCTCTGGGGTGGGATAAGGAAGCCCGGCGGGCAG |
| | | GCATCTTCTCCAGTCCAGCCACGATCTCGCTTCCTCGCTGACACT |
| | | GCCCCTCAGCTACACACGTGTAGCCCTGGGGGCAGCAGTGCTGG |
| | | TGGTCCGAGCAGCAGACAGCCTCTGGGATTGGACAGCAGCCCC |
| | | ACTCCCCAGACGTGAGTTGGCAGCAGGTATCGGAGGAGGGACA |
| | | GCTGCTGACATTATCACAGGGGACATCTCTCTTCAAGGCTTGTG |
| | | GGTCTGGCAGGCTGAGGTGAGCTGGGGCCTTCTCCATCCAGGGC |
| | | ACCTGGTGGGGCCCCTGTTCACAGGTACCCTTCTGCGTGTCACA |
| | | CGTAAACCCCGCGGGACAGCAGTGTATGTGGTCCTCACAGCAC |
| | | ACAGCCTGGGTAAAAGGGCAGCAGCCCCAGGCCCCCGACTGTA |
| | | GACGGCAGCAGGTATAGCCATCTGGGCAGCTCACCTCCATGTCA |
| | | CATTTCACATCCCCCACTGTGTGCGCAGGCAGCTTAGTGAGGAG |
| | | GTCCGTGGTAGCGTTCTCCTTGGAGAGGCACTTACTCTGGATCA |
| | | GGTCACACACAGTGTCTTGGGGGCAGCAGTGCAGGTGATCGGA |
| | | GCAGCAGGTGGCGTTGGGCATTGGGCAGCAGCCATACTTCCCAC |
| | | TGGGCAGCTCACAGCAGGTAGAACCATCAGGGCACCGGGACCG |
| | | TGCGTCCGGACACATGACCGAGCTGGACAAGGCCACTGCCCTGT |
| | | TAGTCCTCTGGGCAGGGAGCTTCTTTGCCAGGGGGTGGGTGCCC |
| | | GTGGGTGTGATGCAGCGGGTGTGAACCAGGTCGCAGAAGGCAC |
| | | CGTGCGGACAGCAGTGCACCCTGTCTTCACAGCAGGAAGCCTG |
| | | GGGCATGGGGCAGCACCCCCAGGAGCCATCGACCATAACACAG |
| | | CACGTGGAGAAGTCCGGGCATTCGAACTGACTATCAGGGCACT |
| | | GGATGGCACCCACGGAGTTGTTACCTGATCTTTGGAAGCAGGAT |
| | | CGCCCGTCTGCACTGCAGTGGAAGCCCCGTGGGCAGCAGTGAT |
| | | GGCCATCCCCGCATGCCACGGCCTCTGGGAAGGGGCAGCAACT |
| | | GGAAGTCCCTGAGACGGTAAAGATGCAGGAGTGGCCGGCAGAG |
| | | CAGTGGGCATCAACCTGGCAGGGGCCACCCAGATGCCTGCTCA |
| | | GTGTTGTGGGCCATTTGTCCAGAAGGGGACGGCAGCAGCTGTA |
| | | GCTGGCTCCTCCGGGGTCCAGGCAGCAGGCCACAGGGCAGAAC |
| | | TGACCATCTGGGCACCGCGTTCCAGCCACCAGCCCTGCTGTTAA |
| | | GGCCACCCAGCTCACCAGGGTCCACATGGTGGCGGCGCTAGCG |
| | | GTGCACACCAATGTGGTGAATGGTCAAATGGCGTTTATTGTATC |
| | | GAGCTAGGCACTTAAATACAATATCTCTGCAATGCGGAATTCAG |
| | | TGGTTCGTCCAATCCATGTCAGACCCGTCTGTTGCCTTCCTAATA |
| | | AGGCACGATCGTACCACCTTACTTCCACCAATCGGCATGCACGG |
| | | TGCTTTTTCTCTCCTTGTAAGGCATGTTGCTAACTCATCGTTACC |
| | | ATGTTGCAAGACTACAAGAGTATTGCATAAGACTACATTGGATC |
| | | CTCTGCGCGCTCGCTCGCTCACTGAGGCCGCCCGGGCAAAGCCC |
| | | GGGCGTCGGGCGACCTTTGGTCGCCCGGCCTCAGTGAGCGAGC |
| | | GAGCGCGCAGAGA |
| SEQ ID | Progranulin Payload | CCAACTGATCTTCAGCATCTTTTACTTTCACCAGCGTTTCTGGGT |
| NO: 147 | Plasmid | GAGCAAAAACAGGAAGGCAAAATGCCGCAAAAAAGGGAATAA |
| | (STX_C0230) | GGGCGACACGGAAATGTTGAATACTCATACTCTTCCTTTTTCAA |
| | | TATTATTGAAGCATTTATCAGGGTTATTGTCTCATGAGCGGATA |
| | | CATATTTGAATGTATTTAGAAAAATAAACAAATAGGGGTTCCGC |
| | | GCACATTTCCCCGAAAAGTGCCACCTAAATTGTAAGCGTTAATA |
| | | TTTTGTTAAAATTCGCGTTAAATTTTTGTTAAATCAGCTCATTTT |
| | | TTAACCAATAGGCCGAAATCGGCAAAATCCCTTATAAATCAAA |
| | | AGAATAGACCGAGATAGGGTTGAGTGTTGTTCCAGTTTGGAACA |
| | | AGAGTCCACTATTAAAGAACGTGGACTCCAACGTCAAAGGGCG |
| | | AAAAACCGTCTATCAGGGCGATGGCCCACTACGTGAACCATCA |
| | | CCCTAATCAAGTTTTTTGGGGTCGAGGTGCCGTAAAGCACTAAA |
| | | TCGGAACCCTAAAGGGAGCCCCCGATTTAGAGCTTGACGGGGA |
| | | AAGCCGGCGAACGTGGCGAGAAAGGAAGGGAAGAAAGCGAAA |
| | | GGAGCGGGCGCTAGGGCGCTGGCAAGTGTAGCGGTCACGCTGC |
| | | GCGTAACCACCACACCCGCCGCGCTTAATGCGCCGCTACAGGGC |
| | | GCGTCCCATTCGCCATTCAGGCTGCGCAACTGTTGGGAAGGGCG |
| | | ATCGGTGCGGGCCTCTTCGCTATTACGCCAGCTGGCGAAAGGGG |
| | | GATGTGCTGCAAGGCGATTAAGTTGGGTAACGCCAGGGTTTTCC |
| | | CAGTCACGACGTTGTAAAACGACGGCCAGTGAGCGCGCCTCGTT |
| | | CATTCACGTTTTTGAACCCGTGGAGGACGGGCAGACTCGCGGTG |
| | | CAAATGTGTTTTACAGCGTGATGGAGCAGATGAAGATGCTCGAC |
| | | ACGCTGCAGAACACGCAGCTAGATTAACCCTAGAAAGATAATC |
| | | ATATTGTGACGTACGTTAAAGATAATCATGTGTAAAATTGACGC |
| | | ATGTGTTTTATCGGTCTGTATATCGAGGTTTATTTATTAATTTGA |
| | | ATAGATATTAAGTTTTATTATATTTACACTTACATACTAATAATA |
| | | AATTCAACAAACAATTTATTTATGTTTATTTATTTATTAAAAAAA |
| | | ACAAAAACTCAAAATTTCTTCTATAAAGTAACAAAACTTTTATT |
| | | TCTTTCCTGCGTTATCTTTGGCCACTCCCTCTCTGCGCGCTCGCT |
| | | CGCTCACTGAGGCCGGGCGACCAAAGGTCGCCCGACGCCCGGG |
| | | CTTTGCCCGGGCGGCCTCAGTGAGCGAGCGAGCGCGCAGAGAG |
| | | GGAGTGGCCAACTCCATCACTAGGGGTTCCTCTGCAGTGAGAGA |
| | | CACAAAAAATTCCAACACACTATTGCAATGAAAATAAATTTCCT |
| | | TTATTTCACAGCAGCTGTCTCAAGGCTGGGTCCCTCAAAGGGGC |
| | | GTCCCAGCGCGGGGCCTCCCTGCGCAAACACTTGGTACCCCTGG |
| | | CTGCGCAGCGGAAGCCAGCAGGACAGCAGTGGCGCCGATCAGC |
| | | ACAACAGACGCCCTGGCGGTAGGGACAGCAGGCCCAGCCCTGT |
| | | CGGTTGTCTCGGCAGCAGGTCTGGTTATCATGGCAGAAGTGTCC |
| | | TTCCCCACACTCCACGTCCTTCACACCCACGTGAGGGCTACGGG |
| | | CCAGGAAGGTGGCAGGCTGGGCAGAGACCACTTCCTTCTCGCA |
| | | GGATCGAGCCTTCACGTTGCAGGTGTAGCCAGCCGGGCAGCAG |
| | | TGCTGGCGATCCTCGCAGCACACAGCATGGGGCAACTGGCAGC |
| | | AGGCCCAGCTCCCACCCAGGCTCGGGCAGCAGGTCTGCCCCACC |
| | | GGGCAGCTGGTGTGCTGGTCACAGCCGATGTCTCTGGGGTGGGA |
| | | TAAGGAAGCCCGGCGGGCAGGCATCTTCTCCAGTCCAGCCACG |
| | | ATCTCGCTTCCTCGCTGACACTGCCCCTCAGCTACACACGTGTA |
| | | GCCCTGGGGGCAGCAGTGCTGGTGGTCCGAGCAGCAGACAGCC |
| | | TCTGGGATTGGACAGCAGCCCCACTCCCCAGACGTGAGTTGGCA |
| | | GCAGGTATCGGAGGAGGGACAGCTGCTGACATTATCACAGGGG |
| | | ACATCTCTCTTCAAGGCTTGTGGGTCTGGCAGGCTGAGGTGAGC |
| | | TGGGGCCTTCTCCATCCAGGGCACCTGGTGGGGCCCCTGTTCAC |
| | | AGGTACCCTTCTGCGTGTCACACGTAAACCCCGCGGGACAGCAG |
| | | TGTATGTGGTCCTCACAGCACACAGCCTGGGTAAAAGGGCAGC |
| | | AGCCCCAGGCCCCCGACTGTAGACGGCAGCAGGTATAGCCATC |
| | | TGGGCAGCTCACCTCCATGTCACATTTCACATCCCCCACTGTGT |
| | | GCGCAGGCAGCTTAGTGAGGAGGTCCGTGGTAGCGTTCTCCTTG |
| | | GAGAGGCACTTACTCTGGATCAGGTCACACACAGTGTCTTGGGG |
| | | GCAGCAGTGCAGGTGATCGGAGCAGCAGGTGGCGTTGGGCATT |
| | | GGGCAGCAGCCATACTTCCCACTGGGCAGCTCACAGCAGGTAG |
| | | AACCATCAGGGCACCGGGACCGTGCGTCCGGACACATGACCGA |
| | | GCTGGACAAGGCCACTGCCCTGTTAGTCCTCTGGGCAGGGAGCT |
| | | TCTTTGCCAGGGGGTGGGTGCCCGTGGGTGTGATGCAGCGGGTG |
| | | TGAACCAGGTCGCAGAAGGCACCGTGCGGACAGCAGTGCACCC |
| | | TGTCTTCACAGCAGGAAGCCTGGGGCATGGGGCAGCACCCCCA |
| | | GGAGCCATCGACCATAACACAGCACGTGGAGAAGTCCGGGCAT |
| | | TCGAACTGACTATCAGGGCACTGGATGGCACCCACGGAGTTGTT |
| | | ACCTGATCTTTGGAAGCAGGATCGCCCGTCTGCACTGCAGTGGA |
| | | AGCCCCGTGGGCAGCAGTGATGGCCATCCCCGCATGCCACGGC |
| | | CTCTGGGAAGGGGCAGCAACTGGAAGTCCCTGAGACGGTAAAG |
| | | ATGCAGGAGTGGCCGGCAGAGCAGTGGGCATCAACCTGGCAGG |
| | | GGCCACCCAGATGCCTGCTCAGTGTTGTGGGCCATTTGTCCAGA |
| | | AGGGGACGGCAGCAGCTGTAGCTGGCTCCTCCGGGGTCCAGGC |
| | | AGCAGGCCACAGGGCAGAACTGACCATCTGGGCACCGCGTTCC |
| | | AGCCACCAGCCCTGCTGTTAAGGCCACCCAGCTCACCAGGGTCC |
| | | ACATGGTGGCGGCGCTAGCGGTGCACACCAATGTGGTGAATGG |
| | | TCAAATGGCGTTTATTGTATCGAGCTAGGCACTTAAATACAATA |
| | | TCTCTGCAATGCGGAATTCAGTGGTTCGTCCAATCCATGTCAGA |
| | | CCCGTCTGTTGCCTTCCTAATAAGGCACGATCGTACCACCTTACT |
| | | TCCACCAATCGGCATGCACGGTGCTTTTTCTCTCCTTGTAAGGCA |
| | | TGTTGCTAACTCATCGTTACCATGTTGCAAGACTACAAGAGTAT |
| | | TGCATAAGACTACATTGGATCCTCTGCGCGCTCGCTCGCTCACT |
| | | GAGGCCGCCCGGGCAAAGCCCGGGCGTCGGGCGACCTTTGGTC |
| | | GCCCGGCCTCAGTGAGCGAGCGAGCGCGCAGAGAATTCACTGG |
| | | CCGTCGTTTTACAACGTCGTGACTGGGAAAACCCTGGCGTTACC |
| | | CAACTTAATCGCCTTGCAGCACATCCCCCTTTCGCCAGCTGGCG |
| | | TAATAGCGAAGAGGCCCGCACCGATCGCCCTTCCCAACAGTTGC |
| | | GCAGCCTGAATGGCGAATGGCGCCTGATGCGGTATTTTCTCCTT |
| | | ACGCATCTGTGCGGTATTTCACACCGCACTAGAGCTAGCGAATT |
| | | CGAATTTAAATCGGATCCGCGGCCGCAAGGATCTGCGATCGCTC |
| | | CGGTGCCCGTCAGTGGGCAGAGCGCACATCGCCCACAGTCCCC |
| | | GAGAAGTTGGGGGGAGGGGTCGGCAATTGAACGGGTGCCTAGA |
| | | GAAGGTGGCGCGGGGTAAACTGGGAAAGTGATGTCGTGTACTG |
| | | GCTCCGCCTTTTTCCCGAGGGTGGGGGAGAACCGTATATAAGTG |
| | | CAGTAGTCGCCGTGAACGTTCTTTTTCGCAACGGGTTTGCCGCC |
| | | AGAACACAGCTGAAGCTTCGAGGGGCTCGCATCTCTCCTTCACG |
| | | CGCCCGCCGCCCTACCTGAGGCCGCCATCCACGCCGGTTGAGTC |
| | | GCGTTCTGCCGCCTCCCGCCTGTGGTGCCTCCTGAACTGCGTCC |
| | | GCCGTCTAGGTAAGTTTAAAGCTCAGGTCGAGACCGGGCCTTTG |
| | | TCCGGCGCTCCCTTGGAGCCTACCTAGACTCAGCCGGCTCTCCA |
| | | CGCTTTGCCTGACCCTGCTTGCTCAACTCTACGTCTTTGTTTCGT |
| | | TTTCTGTTCTGCGCCGTTACAGATCCAAGCTGTGACCGGCGCCT |
| | | ACGATATCGCCACCATGAAAACATTTAACATTTCTCAACAGGAT |
| | | CTAGAATTAGTAGAAGTAGCGACAGAGAAGATTACAATGCTTT |
| | | ATGAGGATAATAAACATCATGTGGGAGCGGCAATTCGTACGAA |
| | | AACAGGAGAAATCATTTCGGCAGTACATATTGAAGCGTATATA |
| | | GGACGAGTAACTGTTTGTGCAGAAGCCATTGCGATTGGTAGTGC |
| | | AGTTTCGAATGGACAAAAGGATTTTGACACGATTGTAGCTGTTA |
| | | GACACCCTTATTCTGACGAAGTAGATAGAAGTATTCGAGTGGTA |
| | | AGTCCTTGTGGTATGTGCCTTTCATACGAGACCGAGATCCTGAC |
| | | TGTCGAGTACGGATTGCTTCCTATCGGCAAAATCGTGGAGAAGA |
| | | GGATTGAATGTACCGTCTATTCAGTCGATAATAATGGGAACATC |
| | | TACACACAGCCCGTGGCTCAATGGCACGACAGAGGAGAGCAGG |
| | | AAGTTTTTGAATACTGTCTCGAGGACGGATCCCTCATCCGCGCT |
| | | ACTAAAGATCATAAGTTTATGACCGTGGACGGCCAGATGCTGCC |
| | | AATTGACGAAATTTTTGAACGAGAGCTGGATCTGATGAGAGTCG |
| | | ACAACCTTCCAAACTGAGTGCATGACCCGCAAGCCCGGTGCCTG |
| | | AAATCAACCTCTGGATTACAAAATTTGTGAAAGATTGACTGGTA |
| | | TTCTTAACTATGTTGCTCCTTTTACGCTATGTGGATACGCTGCTT |
| | | TAATGCCTTTGTATCATGCGTTAACTAAACTTGTTTATTGCAGCT |
| | | TATAATGGTTACAAATAAAGCAATAGCATCACAAATTTCACAAA |
| | | TAAAGCATTTTTTTCACTGCATTCTAGTTGTGGTTTGTCCAAACT |
| | | CATCAATGTATCTTATCATGTCTGGAATTGACTCAAATGATGTC |
| | | AATTAGTCTATCAGAAGCTCATCTGGTCTCCCTTCCGGGGGACA |
| | | AGACATCCCTGTTTAATATTTAAACAGCAGTGTTCCCAAACTGG |
| | | GTTCTTATATCCCTTGCTCTGGTCAACCAGGTTGCAGGGTTTCCT |
| | | GTCCTCACAGGAACGAAGTCCCTAAAGAAACAGTGGCAGCCAG |
| | | GTTTAGCCCCGGAATTGACTGGATTCCTTTTTTAGGGCCCATTGG |
| | | TATGGCTGATATCTATAACAAGAAAATATATATATAATAAGTTA |
| | | TCACGTAAGTAGAACATGAAATAACAATATAATTATCGTATGAG |
| | | TTAAATCTTAAAAGTCACGTAAAAGATAATCATGCGTCATTTTG |
| | | ACTCACGCGGTCGTTATAGTTCAAAATCAGTGACACTTACCGCA |
| | | TTGACAAGCACGCCTCACGGGAGCTCCAAGCGGCGACTGAGAT |
| | | GTCCTAAATGCACAGCGACGGATTCGCGCTATTTAGAAAGAGA |
| | | GAGCAATATTTCAAGAATGCATGCGTCAATTTTACGCAGACTAT |
| | | CTTTCTAGGGTTAATCTAGCTGCATCAGGATCATATCGTCGGGT |
| | | CTTTTTTCCGGCTCAGTCATCGCCCAAGCTGGCGCTATCTGGGC |
| | | ATCGGGGAGGAAGAAGCCCGTGCCTTTTCCCGCGAGGTTGAAG |
| | | CGGCATGGAAAGAGTTTGCCGAGGATGACTGCTGCTGCATTGAC |
| | | GTTGAGCGAAAACGCACGTTTACCATGATGATTCGGGAAGGTGT |
| | | GGCCATGCACGCCTTTAACGGTGAACTGTTCGTTCAGGCCACCT |
| | | GGGATACCAGTTCGTCGCGGCTTTTCCGGACACAGTTCCGGATG |
| | | GTCAGCCCGAAGCGCATCAGCAACCCGAACAATACCGGCGACA |
| | | GCCGGAACTGCCGTGCCGGTGTGCAGATTAATGACAGCGGTGC |
| | | GGCGCTGGGATATTACGTCAGCGAGGACGGGTATCCTGGCTGG |
| | | ATGCCGCAGAAATGGACATGGATACCCCGTGAGTTACCCGGCG |
| | | GGCGCGCTTGGCGTAATCATGGTCATAGCTGTTTCCTGTGTGAA |
| | | ATTGTTATCCGCTCACAATTCCACACAACATACGAGCCGGAAGC |
| | | ATAAAGTGTAAAGCCTGGGGTGCCTAATGAGTGAGCTAACTCA |
| | | CATTAATTGCGTTGCGCTCACTGCCCGCTTTCCAGTCGGGAAAC |
| | | CTGTCGTGCCAGCTGCATTAATGAATCGGCCAACGCGCGGGGA |
| | | GAGGCGGTTTGCGTATTGGGCGCTCTTCCGCTTCCTCGCTCACTG |
| | | ACTCGCTGCGCTCGGTCGTTCGGCTGCGGCGAGCGGTATCAGCT |
| | | CACTCAAAGGCGGTAATACGGTTATCCACAGAATCAGGGGATA |
| | | ACGCAGGAAAGAACATGTGAGCAAAAGGCCAGCAAAAGGCCA |
| | | GGAACCGTAAAAAGGCCGCGTTGCTGGCGTTTTTCCATAGGCTC |
| | | CGCCCCCCTGACGAGCATCACAAAAATCGACGCTCAAGTCAGA |
| | | GGTGGCGAAACCCGACAGGACTATAAAGATACCAGGCGTTTCC |
| | | CCCTGGAAGCTCCCTCGTGCGCTCTCCTGTTCCGACCCTGCCGCT |
| | | TACCGGATACCTGTCCGCCTTTCTCCCTTCGGGAAGCGTGGCGC |
| | | TTTCTCATAGCTCACGCTGTAGGTATCTCAGTTCGGTGTAGGTCG |
| | | TTCGCTCCAAGCTGGGCTGTGTGCACGAACCCCCCGTTCAGCCC |
| | | GACCGCTGCGCCTTATCCGGTAACTATCGTCTTGAGTCCAACCC |
| | | GGTAAGACACGACTTATCGCCACTGGCAGCAGCCACTGGTAAC |
| | | AGGATTAGCAGAGCGAGGTATGTAGGCGGTGCTACAGAGTTCT |
| | | TGAAGTGGTGGCCTAACTACGGCTACACTAGAAGAACAGTATTT |
| | | GGTATCTGCGCTCTGCTGAAGCCAGTTACCTTCGGAAAAAGAGT |
| | | TGGTAGCTCTTGATCCGGCAAACAAACCACCGCTGGTAGCGGTG |
| | | GTTTTTTTGTTTGCAAGCAGCAGATTACGCGCAGAAAAAAAGGA |
| | | TCTCAAGAAGATCCTTTGATCTTTTCTACGGGGTCTGACGCTCA |
| | | GTGGAACGAAAACTCACGTTAAGGGATTTTGGTCATGAGATTAT |
| | | CAAAAAGGATCTTCACCTAGATCCTTTTAAATTAAAAATGAAGT |
| | | TTTAAATCAATCTAAAGTATATATGAGTAAACTTGGTCTGACAG |
| | | TTACCAATGCTTAATCAGTGAGGCACCTATCTCAGCGATCTGTC |
| | | TATTTCGTTCATCCATAGTTGCCTGACTCCCCGTCGTGTAGATAA |
| | | CTACGATACGGGAGGGCTTACCATCTGGCCCCAGTGCTGCAATG |
| | | ATACCGCGAGACCCACGCTCACCGGCTCCAGATTTATCAGCAAT |
| | | AAACCAGCCAGCCGGAAGGGCCGAGCGCAGAAGTGGTCCTGCA |
| | | ACTTTATCCGCCTCCATCCAGTCTATTAATTGTTGCCGGGAAGCT |
| | | AGAGTAAGTAGTTCGCCAGTTAATAGTTTGCGCAACGTTGTTGC |
| | | CATTGCTACAGGCATCGTGGTGTCACGCTCGTCGTTTGGTATGG |
| | | CTTCATTCAGCTCCGGTTCCCAACGATCAAGGCGAGTTACATGA |
| | | TCCCCCATGTTGTGCAAAAAAGCGGTTAGCTCCTTCGGTCCTCC |
| | | GATCGTTGTCAGAAGTAAGTTGGCCGCAGTGTTATCACTCATGG |
| | | TTATGGCAGCACTGCATAATTCTCTTACTGTCATGCCATCCGTAA |
| | | GATGCTTTTCTGTGACTGGTGAGTACTCAACCAAGTCATTCTGA |
| | | GAATAGTGTATGCGGCGACCGAGTTGCTCTTGCCCGGCGTCAAT |
| | | ACGGGATAATACCGCGCCACATAGCAGAACTTTAAAAGTGCTC |
| | | ATCATTGGAAAACGTTCTTCGGGGCGAAAACTCTCAAGGATCTT |
| | | ACCGCTGTTGAGATCCAGTTCGATGTAACCCACTCGTGCAC |
| 148 | Progranulin payload | TCTCTGCGCGCTCGCTCGCTCACTGAGGCCGGGCGACCAAAGGT |
| | (ITR to ITR) (of | CGCCCGACGCCCGGGCTTTGCCCGGGCGGCCTCAGTGAGCGAG |
| | SEQ ID NO: 149) | CGAGCGCGCAGAGGATCCGGGCGGAGTTAGGGCGGAGCCAATC |
| | | AGCGTGCGCCGTTCCGAAAGTTGCCTTTTATGGCTGGGCGGAGA |
| | | ATGGGCGGTGAACGCCGATGATTATATAAGGACGCGCCGGGTG |
| | | TGGCACAGCTAGTTCCGTCGCAGCCGGGATTTGGGTCGCGGTTC |
| | | TTGTTTGTGGATCCCTGTGATCGTCACTTGGCTAGCGCCGCCACC |
| | | ATGTGGACCCTGGTGAGCTGGGTGGCCTTAACAGCAGGGCTGGT |
| | | GGCTGGAACGCGGTGCCCAGATGGTCAGTTCTGCCCTGTGGCCT |
| | | GCTGCCTGGACCCCGGAGGAGCCAGCTACAGCTGCTGCCGTCCC |
| | | CTTCTGGACAAATGGCCCACAACACTGAGCAGGCATCTGGGTG |
| | | GCCCCTGCCAGGTTGATGCCCACTGCTCTGCCGGCCACTCCTGC |
| | | ATCTTTACCGTCTCAGGGACTTCCAGTTGCTGCCCCTTCCCAGAG |
| | | GCCGTGGCATGCGGGGATGGCCATCACTGCTGCCCACGGGGCTT |
| | | CCACTGCAGTGCAGACGGGCGATCCTGCTTCCAAAGATCAGGTA |
| | | ACAACTCCGTGGGTGCCATCCAGTGCCCTGATAGTCAGTTCGAA |
| | | TGCCCGGACTTCTCCACGTGCTGTGTTATGGTCGATGGCTCCTG |
| | | GGGGTGCTGCCCCATGCCCCAGGCTTCCTGCTGTGAAGACAGGG |
| | | TGCACTGCTGTCCGCACGGTGCCTTCTGCGACCTGGTTCACACC |
| | | CGCTGCATCACACCCACGGGCACCCACCCCCTGGCAAAGAAGC |
| | | TCCCTGCCCAGAGGACTAACAGGGCAGTGGCCTTGTCCAGCTCG |
| | | GTCATGTGTCCGGACGCACGGTCCCGGTGCCCTGATGGTTCTAC |
| | | CTGCTGTGAGCTGCCCAGTGGGAAGTATGGCTGCTGCCCAATGC |
| | | CCAACGCCACCTGCTGCTCCGATCACCTGCACTGCTGCCCCCAA |
| | | GACACTGTGTGTGACCTGATCCAGAGTAAGTGCCTCTCCAAGGA |
| | | GAACGCTACCACGGACCTCCTCACTAAGCTGCCTGCGCACACAG |
| | | TGGGGGATGTGAAATGTGACATGGAGGTGAGCTGCCCAGATGG |
| | | CTATACCTGCTGCCGTCTACAGTCGGGGGCCTGGGGCTGCTGCC |
| | | CTTTTACCCAGGCTGTGTGCTGTGAGGACCACATACACTGCTGT |
| | | CCCGCGGGGTTTACGTGTGACACGCAGAAGGGTACCTGTGAAC |
| | | AGGGGCCCCACCAGGTGCCCTGGATGGAGAAGGCCCCAGCTCA |
| | | CCTCAGCCTGCCAGACCCACAAGCCTTGAAGAGAGATGTCCCCT |
| | | GTGATAATGTCAGCAGCTGTCCCTCCTCCGATACCTGCTGCCAA |
| | | CTCACGTCTGGGGAGTGGGGCTGCTGTCCAATCCCAGAGGCTGT |
| | | CTGCTGCTCGGACCACCAGCACTGCTGCCCCCAGGGCTACACGT |
| | | GTGTAGCTGAGGGGCAGTGTCAGCGAGGAAGCGAGATCGTGGC |
| | | TGGACTGGAGAAGATGCCTGCCCGCCGGGCTTCCTTATCCCACC |
| | | CCAGAGACATCGGCTGTGACCAGCACACCAGCTGCCCGGTGGG |
| | | GCAGACCTGCTGCCCGAGCCTGGGTGGGAGCTGGGCCTGCTGCC |
| | | AGTTGCCCCATGCTGTGTGCTGCGAGGATCGCCAGCACTGCTGC |
| | | CCGGCTGGCTACACCTGCAACGTGAAGGCTCGATCCTGCGAGA |
| | | AGGAAGTGGTCTCTGCCCAGCCTGCCACCTTCCTGGCCCGTAGC |
| | | CCTCACGTGGGTGTGAAGGACGTGGAGTGTGGGGAAGGACACT |
| | | TCTGCCATGATAACCAGACCTGCTGCCGAGACAACCGACAGGG |
| | | CTGGGCCTGCTGTCCCTACCGCCAGGGCGTCTGTTGTGCTGATC |
| | | GGCGCCACTGCTGTCCTGCTGGCTTCCGCTGCGCAGCCAGGGGT |
| | | ACCAAGTGTTTGCGCAGGGAGGCCCCGCGCTGGGACGCCCCTTT |
| | | GAGGGACCCAGCCTTGAGACAGCTGCTGTGAAATAAAGGAAAT |
| | | TTATTTTCATTGCAATAGTGTGTTGGAATTTTTTGTGTCTCTCAC |
| | | TGCAGAGGAACCCCTAGTGATGGAGTTGGCCACTCCCTCTCTGC |
| | | GCGCTCGCTCGCTCACTGAGGCCGGGCGACCAAAGGTCGCCCG |
| | | ACGCCCGGGCTTTGCCCGGGCGGCCTCAGTGAGCGAGCGAGCG |
| | | CGCAG |
| 149 | Progranulin Payload | AGCAACTCGGTCGCCGCATACACTATTCTCAGAATGACTTGGTT |
| | Plasmid | GAGTACTCACCAGTCACAGAAAAGCATCTTACGGATGGCATGA |
| | (STX_C0229) | CAGTAAGAGAATTATGCAGTGCTGCCATAACCATGAGTGATAA |
| | | CACTGCGGCCAACTTACTTCTGACAACGATCGGAGGACCGAAG |
| | | GAGCTAACCGCTTTTTTGCACAACATGGGGGATCATGTAACTCG |
| | | CCTTGATCGTTGGGAACCGGAGCTGAATGAAGCCATACCAAAC |
| | | GACGAGCGTGACACCACGATGCCTGTAGCAATGGCAACAACGT |
| | | TGCGCAAACTATTAACTGGCGAACTACTTACTCTAGCTTCCCGG |
| | | CAACAATTAATAGACTGGATGGAGGCGGATAAAGTTGCAGGAC |
| | | CACTTCTGCGCTCGGCCCTTCCGGCTGGCTGGTTTATTGCTGATA |
| | | AATCTGGAGCCGGTGAGCGTGGGTCTCGCGGTATCATTGCAGCA |
| | | CTGGGGCCAGATGGTAAGCCCTCCCGTATCGTAGTTATCTACAC |
| | | GACGGGGAGTCAGGCAACTATGGATGAACGAAATAGACAGATC |
| | | GCTGAGATAGGTGCCTCACTGATTAAGCATTGGTAACTGTCAGA |
| | | CCAAGTTTACTCATATATACTTTAGATTGATTTAAAACTTCATTT |
| | | TTAATTTAAAAGGATCTAGGTGAAGATCCTTTTTGATAATCTCA |
| | | TGACCAAAATCCCTTAACGTGAGTTTTCGTTCCACTGAGCGTCA |
| | | GACCCCGTAGAAAAGATCAAAGGATCTTCTTGAGATCCTTTTTT |
| | | TCTGCGCGTAATCTGCTGCTTGCAAACAAAAAAACCACCGCTAC |
| | | CAGCGGTGGTTTGTTTGCCGGATCAAGAGCTACCAACTCTTTTT |
| | | CCGAAGGTAACTGGCTTCAGCAGAGCGCAGATACCAAATACTG |
| | | TTCTTCTAGTGTAGCCGTAGTTAGGCCACCACTTCAAGAACTCT |
| | | GTAGCACCGCCTACATACCTCGCTCTGCTAATCCTGTTACCAGT |
| | | GGCTGCTGCCAGTGGCGATAAGTCGTGTCTTACCGGGTTGGACT |
| | | CAAGACGATAGTTACCGGATAAGGCGCAGCGGTCGGGCTGAAC |
| | | GGGGGGTTCGTGCACACAGCCCAGCTTGGAGCGAACGACCTAC |
| | | ACCGAACTGAGATACCTACAGCGTGAGCTATGAGAAAGCGCCA |
| | | CGCTTCCCGAAGGGAGAAAGGCGGACAGGTATCCGGTAAGCGG |
| | | CAGGGTCGGAACAGGAGAGCGCACGAGGGAGCTTCCAGGGGG |
| | | AAACGCCTGGTATCTTTATAGTCCTGTCGGGTTTCGCCACCTCTG |
| | | ACTTGAGCGTCGATTTTTGTGATGCTCGTCAGGGGGGCGGAGCC |
| | | TATGGAAAAACGCCAGCAACGCGGCCTTTTTACGGTTCCTGGCC |
| | | TTTTGCTGGCCTTTTGCTCACATGTTCTTTCCTGCGTTATCCCCTG |
| | | ATTCTGTGGATAACCGTATTACCGCCTTTGAGTGAGCTGATACC |
| | | GCTCGCCGCAGCCGAACGACCGAGCGCAGCGAGTCAGTGAGCG |
| | | AGGAAGCGGAAGAGCGCCCAATACGCAAACCGCCTCTCCCCGC |
| | | GCGTTGGCCGATTCATTAATGCAGCTGGCACGACAGGTTTCCCG |
| | | ACTGGAAAGCGGGCAGTGAGCGCAACGCAATTAATGTGAGTTA |
| | | GCTCACTCATTAGGCACCCCAGGCTTTACACTTTATGCTTCCGGC |
| | | TCGTATGTTGTGTGGAATTGTGAGCGGATAACAATTTCACACAG |
| | | GAAACAGCTATGACCATGATTACGCCAAGCGCGCCCGCCGGGT |
| | | AACTCACGGGGTATCCATGTCCATTTCTGCGGCATCCAGCCAGG |
| | | ATACCCGTCCTCGCTGACGTAATATCCCAGCGCCGCACCGCTGT |
| | | CATTAATCTGCACACCGGCACGGCAGTTCCGGCTGTCGCCGGTA |
| | | TTGTTCGGGTTGCTGATGCGCTTCGGGCTGACCATCCGGAACTG |
| | | TGTCCGGAAAAGCCGCGACGAACTGGTATCCCAGGTGGCCTGA |
| | | ACGAACAGTTCACCGTTAAAGGCGTGCATGGCCACACCTTCCCG |
| | | AATCATCATGGTAAACGTGCGTTTTCGCTCAACGTCAATGCAGC |
| | | AGCAGTCATCCTCGGCAAACTCTTTCCATGCCGCTTCAACCTCG |
| | | CGGGAAAAGGCACGGGCTTCTTCCTCCCCGATGCCCAGATAGCG |
| | | CCAGCTTGGGCGATGACTGAGCCGGAAAAAAGACCCGACGATA |
| | | TGATCCTGATGCAGCTAGATTAACCCTAGAAAGATAGTCTGCGT |
| | | AAAATTGACGCATGCATTCTTGAAATATTGCTCTCTCTTTCTAAA |
| | | TAGCGCGAATCCGTCGCTGTGCATTTAGGACATCTCAGTCGCCG |
| | | CTTGGAGCTCCCGTGAGGCGTGCTTGTCAATGCGGTAAGTGTCA |
| | | CTGATTTTGAACTATAACGACCGCGTGAGTCAAAATGACGCATG |
| | | ATTATCTTTTACGTGACTTTTAAGATTTAACTCATACGATAATTA |
| | | TATTGTTATTTCATGTTCTACTTACGTGATAACTTATTATATATA |
| | | TATTTTCTTGTTATAGATATCAGCCATACCAATGGGCCCTAAAA |
| | | AAGGAATCCAGTCAATTCCGGGGCTAAACCTGGCTGCCACTGTT |
| | | TCTTTAGGGACTTCGTTCCTGTGAGGACAGGAAACCCTGCAACC |
| | | TGGTTGACCAGAGCAAGGGATATAAGAACCCAGTTTGGGAACA |
| | | CTGCTGTTTAAATATTAAACAGGGATGTCTTGTCCCCCGGAAGG |
| | | GAGACCAGATGAGCTTCTGATAGACTAATTGACATCATTTGAGT |
| | | CAATTCCAGACATGATAAGATACATTGATGAGTTTGGACAAACC |
| | | ACAACTAGAATGCAGTGAAAAAAATGCTTTATTTGTGAAATTTG |
| | | TGATGCTATTGCTTTATTTGTAACCATTATAAGCTGCAATAAAC |
| | | AAGTTTAGTTAACGCATGATACAAAGGCATTAAAGCAGCGTATC |
| | | CACATAGCGTAAAAGGAGCAACATAGTTAAGAATACCAGTCAA |
| | | TCTTTCACAAATTTTGTAATCCAGAGGTTGATTTCAGGCACCGG |
| | | GCTTGCGGGTCATGCACTCAGTTTGGAAGGTTGTCGACTCTCAT |
| | | CAGATCCAGCTCTCGTTCAAAAATTTCGTCAATTGGCAGCATCT |
| | | GGCCGTCCACGGTCATAAACTTATGATCTTTAGTAGCGCGGATG |
| | | AGGGATCCGTCCTCGAGACAGTATTCAAAAACTTCCTGCTCTCC |
| | | TCTGTCGTGCCATTGAGCCACGGGCTGTGTGTAGATGTTCCCAT |
| | | TATTATCGACTGAATAGACGGTACATTCAATCCTCTTCTCCACG |
| | | ATTTTGCCGATAGGAAGCAATCCGTACTCGACAGTCAGGATCTC |
| | | GGTCTCGTATGAAAGGCACATACCACAAGGACTTACCACTCGA |
| | | ATACTTCTATCTACTTCGTCAGAATAAGGGTGTCTAACAGCTAC |
| | | AATCGTGTCAAAATCCTTTTGTCCATTCGAAACTGCACTACCAA |
| | | TCGCAATGGCTTCTGCACAAACAGTTACTCGTCCTATATACGCT |
| | | TCAATATGTACTGCCGAAATGATTTCTCCTGTTTTCGTACGAATT |
| | | GCCGCTCCCACATGATGTTTATTATCCTCATAAAGCATTGTAATC |
| | | TTCTCTGTCGCTACTTCTACTAATTCTAGATCCTGTTGAGAAATG |
| | | TTAAATGTTTTCATGGTGGCGATATCGTAGGCGCCGGTCACAGC |
| | | TTGGATCTGTAACGGCGCAGAACAGAAAACGAAACAAAGACGT |
| | | AGAGTTGAGCAAGCAGGGTCAGGCAAAGCGTGGAGAGCCGGCT |
| | | GAGTCTAGGTAGGCTCCAAGGGAGCGCCGGACAAAGGCCCGGT |
| | | CTCGACCTGAGCTTTAAACTTACCTAGACGGCGGACGCAGTTCA |
| | | GGAGGCACCACAGGCGGGAGGCGGCAGAACGCGACTCAACCG |
| | | GCGTGGATGGCGGCCTCAGGTAGGGCGGCGGGCGCGTGAAGGA |
| | | GAGATGCGAGCCCCTCGAAGCTTCAGCTGTGTTCTGGCGGCAAA |
| | | CCCGTTGCGAAAAAGAACGTTCACGGCGACTACTGCACTTATAT |
| | | ACGGTTCTCCCCCACCCTCGGGAAAAAGGCGGAGCCAGTACAC |
| | | GACATCACTTTCCCAGTTTACCCCGCGCCACCTTCTCTAGGCACC |
| | | CGTTCAATTGCCGACCCCTCCCCCCAACTTCTCGGGGACTGTGG |
| | | GCGATGTGCGCTCTGCCCACTGACGGGCACCGGAGCGATCGCA |
| | | GATCCTTGCGGCCGCGGATCCGATTTAAATTCGAATTCGCTAGC |
| | | TCTAGTGCGGTGTGAAATACCGCACAGATGCGTAAGGAGAAAA |
| | | TACCGCATCAGGCGCCATTCGCCATTCAGGCTGCGCAACTGTTG |
| | | GGAAGGGCGATCGGTGCGGGCCTCTTCGCTATTACGCCAGCTGG |
| | | CGAAAGGGGGATGTGCTGCAAGGCGATTAAGTTGGGTAACGCC |
| | | AGGGTTTTCCCAGTCACGACGTTGTAAAACGACGGCCAGTGAAT |
| | | TCTCTGCGCGCTCGCTCGCTCACTGAGGCCGGGCGACCAAAGGT |
| | | CGCCCGACGCCCGGGCTTTGCCCGGGCGGCCTCAGTGAGCGAG |
| | | CGAGCGCGCAGAGGATCCGGGCGGAGTTAGGGCGGAGCCAATC |
| | | AGCGTGCGCCGTTCCGAAAGTTGCCTTTTATGGCTGGGCGGAGA |
| | | ATGGGCGGTGAACGCCGATGATTATATAAGGACGCGCCGGGTG |
| | | TGGCACAGCTAGTTCCGTCGCAGCCGGGATTTGGGTCGCGGTTC |
| | | TTGTTTGTGGATCCCTGTGATCGTCACTTGGCTAGCGCCGCCACC |
| | | ATGTGGACCCTGGTGAGCTGGGTGGCCTTAACAGCAGGGCTGGT |
| | | GGCTGGAACGCGGTGCCCAGATGGTCAGTTCTGCCCTGTGGCCT |
| | | GCTGCCTGGACCCCGGAGGAGCCAGCTACAGCTGCTGCCGTCCC |
| | | CTTCTGGACAAATGGCCCACAACACTGAGCAGGCATCTGGGTG |
| | | GCCCCTGCCAGGTTGATGCCCACTGCTCTGCCGGCCACTCCTGC |
| | | ATCTTTACCGTCTCAGGGACTTCCAGTTGCTGCCCCTTCCCAGAG |
| | | GCCGTGGCATGCGGGGATGGCCATCACTGCTGCCCACGGGGCTT |
| | | CCACTGCAGTGCAGACGGGCGATCCTGCTTCCAAAGATCAGGTA |
| | | ACAACTCCGTGGGTGCCATCCAGTGCCCTGATAGTCAGTTCGAA |
| | | TGCCCGGACTTCTCCACGTGCTGTGTTATGGTCGATGGCTCCTG |
| | | GGGGTGCTGCCCCATGCCCCAGGCTTCCTGCTGTGAAGACAGGG |
| | | TGCACTGCTGTCCGCACGGTGCCTTCTGCGACCTGGTTCACACC |
| | | CGCTGCATCACACCCACGGGCACCCACCCCCTGGCAAAGAAGC |
| | | TCCCTGCCCAGAGGACTAACAGGGCAGTGGCCTTGTCCAGCTCG |
| | | GTCATGTGTCCGGACGCACGGTCCCGGTGCCCTGATGGTTCTAC |
| | | CTGCTGTGAGCTGCCCAGTGGGAAGTATGGCTGCTGCCCAATGC |
| | | CCAACGCCACCTGCTGCTCCGATCACCTGCACTGCTGCCCCCAA |
| | | GACACTGTGTGTGACCTGATCCAGAGTAAGTGCCTCTCCAAGGA |
| | | GAACGCTACCACGGACCTCCTCACTAAGCTGCCTGCGCACACAG |
| | | TGGGGGATGTGAAATGTGACATGGAGGTGAGCTGCCCAGATGG |
| | | CTATACCTGCTGCCGTCTACAGTCGGGGGCCTGGGGCTGCTGCC |
| | | CTTTTACCCAGGCTGTGTGCTGTGAGGACCACATACACTGCTGT |
| | | CCCGCGGGGTTTACGTGTGACACGCAGAAGGGTACCTGTGAAC |
| | | AGGGGCCCCACCAGGTGCCCTGGATGGAGAAGGCCCCAGCTCA |
| | | CCTCAGCCTGCCAGACCCACAAGCCTTGAAGAGAGATGTCCCCT |
| | | GTGATAATGTCAGCAGCTGTCCCTCCTCCGATACCTGCTGCCAA |
| | | CTCACGTCTGGGGAGTGGGGCTGCTGTCCAATCCCAGAGGCTGT |
| | | CTGCTGCTCGGACCACCAGCACTGCTGCCCCCAGGGCTACACGT |
| | | GTGTAGCTGAGGGGCAGTGTCAGCGAGGAAGCGAGATCGTGGC |
| | | TGGACTGGAGAAGATGCCTGCCCGCCGGGCTTCCTTATCCCACC |
| | | CCAGAGACATCGGCTGTGACCAGCACACCAGCTGCCCGGTGGG |
| | | GCAGACCTGCTGCCCGAGCCTGGGTGGGAGCTGGGCCTGCTGCC |
| | | AGTTGCCCCATGCTGTGTGCTGCGAGGATCGCCAGCACTGCTGC |
| | | CCGGCTGGCTACACCTGCAACGTGAAGGCTCGATCCTGCGAGA |
| | | AGGAAGTGGTCTCTGCCCAGCCTGCCACCTTCCTGGCCCGTAGC |
| | | CCTCACGTGGGTGTGAAGGACGTGGAGTGTGGGGAAGGACACT |
| | | TCTGCCATGATAACCAGACCTGCTGCCGAGACAACCGACAGGG |
| | | CTGGGCCTGCTGTCCCTACCGCCAGGGCGTCTGTTGTGCTGATC |
| | | GGCGCCACTGCTGTCCTGCTGGCTTCCGCTGCGCAGCCAGGGGT |
| | | ACCAAGTGTTTGCGCAGGGAGGCCCCGCGCTGGGACGCCCCTTT |
| | | GAGGGACCCAGCCTTGAGACAGCTGCTGTGAAATAAAGGAAAT |
| | | TTATTTTCATTGCAATAGTGTGTTGGAATTTTTTGTGTCTCTCAC |
| | | TGCAGAGGAACCCCTAGTGATGGAGTTGGCCACTCCCTCTCTGC |
| | | GCGCTCGCTCGCTCACTGAGGCCGGGCGACCAAAGGTCGCCCG |
| | | ACGCCCGGGCTTTGCCCGGGCGGCCTCAGTGAGCGAGCGAGCG |
| | | CGCAGAGAGGGAGTGGCCAAAGATAACGCAGGAAAGAAATAA |
| | | AAGTTTTGTTACTTTATAGAAGAAATTTTGAGTTTTTGTTTTTTTT |
| | | AATAAATAAATAAACATAAATAAATTGTTTGTTGAATTTATTAT |
| | | TAGTATGTAAGTGTAAATATAATAAAACTTAATATCTATTCAAA |
| | | TTAATAAATAAACCTCGATATACAGACCGATAAAACACATGCGT |
| | | CAATTTTACACATGATTATCTTTAACGTACGTCACAATATGATTA |
| | | TCTTTCTAGGGTTAATCTAGCTGCGTGTTCTGCAGCGTGTCGAGC |
| | | ATCTTCATCTGCTCCATCACGCTGTAAAACACATTTGCACCGCG |
| | | AGTCTGCCCGTCCTCCACGGGTTCAAAAACGTGAATGAACGAG |
| | | GCGCGCTCACTGGCCGTCGTTTTACAACGTCGTGACTGGGAAAA |
| | | CCCTGGCGTTACCCAACTTAATCGCCTTGCAGCACATCCCCCTTT |
| | | CGCCAGCTGGCGTAATAGCGAAGAGGCCCGCACCGATCGCCCT |
| | | TCCCAACAGTTGCGCAGCCTGAATGGCGAATGGGACGCGCCCT |
| | | GTAGCGGCGCATTAAGCGCGGCGGGTGTGGTGGTTACGCGCAG |
| | | CGTGACCGCTACACTTGCCAGCGCCCTAGCGCCCGCTCCTTTCG |
| | | CTTTCTTCCCTTCCTTTCTCGCCACGTTCGCCGGCTTTCCCCGTCA |
| | | AGCTCTAAATCGGGGGCTCCCTTTAGGGTTCCGATTTAGTGCTTT |
| | | ACGGCACCTCGACCCCAAAAAACTTGATTAGGGTGATGGTTCAC |
| | | GTAGTGGGCCATCGCCCTGATAGACGGTTTTTCGCCCTTTGACG |
| | | TTGGAGTCCACGTTCTTTAATAGTGGACTCTTGTTCCAAACTGG |
| | | AACAACACTCAACCCTATCTCGGTCTATTCTTTTGATTTATAAGG |
| | | GATTTTGCCGATTTCGGCCTATTGGTTAAAAAATGAGCTGATTT |
| | | AACAAAAATTTAACGCGAATTTTAACAAAATATTAACGCTTACA |
| | | ATTTAGGTGGCACTTTTCGGGGAAATGTGCGCGGAACCCCTATT |
| | | TGTTTATTTTTCTAAATACATTCAAATATGTATCCGCTCATGAGA |
| | | CAATAACCCTGATAAATGCTTCAATAATATTGAAAAAGGAAGA |
| | | GTATGAGTATTCAACATTTCCGTGTCGCCCTTATTCCCTTTTTTG |
| | | CGGCATTTTGCCTTCCTGTTTTTGCTCACCCAGAAACGCTGGTGA |
| | | AAGTAAAAGATGCTGAAGATCAGTTGGGTGCACGAGTGGGTTA |
| | | CATCGAACTGGATCTCAACAGCGGTAAGATCCTTGAGAGTTTTC |
| | | GCCCCGAAGAACGTTTTCCAATGATGAGCACTTTTAAAGTTCTG |
| | | CTATGTGGCGCGGTATTATCCCGTATTGACGCCGGGCAAG |
| 150 | Progranulin payload | TCTCTGCGCGCTCGCTCGCTCACTGAGGCCGGGCGACCAAAGGT |
| | (ITR to ITR) (of | CGCCCGACGCCCGGGCTTTGCCCGGGCGGCCTCAGTGAGCGAG |
| | SEQ ID NO: 151) | CGAGCGCGCAGAGGATCCAATGTAGTCTTATGCAATACTCTTGT |
| | | AGTCTTGCAACATGGTAACGATGAGTTAGCAACATGCCTTACAA |
| | | GGAGAGAAAAAGCACCGTGCATGCCGATTGGTGGAAGTAAGGT |
| | | GGTACGATCGTGCCTTATTAGGAAGGCAACAGACGGGTCTGAC |
| | | ATGGATTGGACGAACCACTGAATTCCGCATTGCAGAGATATTGT |
| | | ATTTAAGTGCCTAGCTCGATACAATAAACGCCATTTGACCATTC |
| | | ACCACATTGGTGTGCACCGCTAGCGCCGCCACCATGTGGACCCT |
| | | GGTGAGCTGGGTGGCCTTAACAGCAGGGCTGGTGGCTGGAACG |
| | | CGGTGCCCAGATGGTCAGTTCTGCCCTGTGGCCTGCTGCCTGGA |
| | | CCCCGGAGGAGCCAGCTACAGCTGCTGCCGTCCCCTTCTGGACA |
| | | AATGGCCCACAACACTGAGCAGGCATCTGGGTGGCCCCTGCCA |
| | | GGTTGATGCCCACTGCTCTGCCGGCCACTCCTGCATCTTTACCG |
| | | TCTCAGGGACTTCCAGTTGCTGCCCCTTCCCAGAGGCCGTGGCA |
| | | TGCGGGGATGGCCATCACTGCTGCCCACGGGGCTTCCACTGCAG |
| | | TGCAGACGGGCGATCCTGCTTCCAAAGATCAGGTAACAACTCC |
| | | GTGGGTGCCATCCAGTGCCCTGATAGTCAGTTCGAATGCCCGGA |
| | | CTTCTCCACGTGCTGTGTTATGGTCGATGGCTCCTGGGGGTGCT |
| | | GCCCCATGCCCCAGGCTTCCTGCTGTGAAGACAGGGTGCACTGC |
| | | TGTCCGCACGGTGCCTTCTGCGACCTGGTTCACACCCGCTGCAT |
| | | CACACCCACGGGCACCCACCCCCTGGCAAAGAAGCTCCCTGCC |
| | | CAGAGGACTAACAGGGCAGTGGCCTTGTCCAGCTCGGTCATGT |
| | | GTCCGGACGCACGGTCCCGGTGCCCTGATGGTTCTACCTGCTGT |
| | | GAGCTGCCCAGTGGGAAGTATGGCTGCTGCCCAATGCCCAACG |
| | | CCACCTGCTGCTCCGATCACCTGCACTGCTGCCCCCAAGACACT |
| | | GTGTGTGACCTGATCCAGAGTAAGTGCCTCTCCAAGGAGAACG |
| | | CTACCACGGACCTCCTCACTAAGCTGCCTGCGCACACAGTGGGG |
| | | GATGTGAAATGTGACATGGAGGTGAGCTGCCCAGATGGCTATA |
| | | CCTGCTGCCGTCTACAGTCGGGGGCCTGGGGCTGCTGCCCTTTT |
| | | ACCCAGGTACCCAGGGGTGGCGGGTGGGTGGGCTGAGCACAGT |
| | | GTGGCAGGCAGCCGGGCCCCAGTGCCCACCTGCCCTTCTTCATC |
| | | TGCCCTAGGCTGTGTGCTGTGAGGACCACATACACTGCTGTCCC |
| | | GCGGGGTTTACGTGTGACACGCAGAAGGGTACCTGTGAACAGG |
| | | GGCCCCACCAGGTGCCCTGGATGGAGAAGGCCCCAGCTCACCT |
| | | CAGCCTGCCAGACCCACAAGCCTTGAAGAGAGATGTCCCCTGT |
| | | GATAATGTCAGCAGCTGTCCCTCCTCCGATACCTGCTGCCAACT |
| | | CACGTCTGGGGAGTGGGGCTGCTGTCCAATCCCAGAGGCTGTCT |
| | | GCTGCTCGGACCACCAGCACTGCTGCCCCCAGGGCTACACGTGT |
| | | GTAGCTGAGGGGCAGTGTCAGCGAGGAAGCGAGATCGTGGCTG |
| | | GACTGGAGAAGATGCCTGCCCGCCGGGCTTCCTTATCCCACCCC |
| | | AGAGACATCGGCTGTGACCAGCACACCAGCTGCCCGGTGGGGC |
| | | AGACCTGCTGCCCGAGCCTGGGTGGGAGCTGGGCCTGCTGCCA |
| | | GTTGCCCCATGCTGTGTGCTGCGAGGATCGCCAGCACTGCTGCC |
| | | CGGCTGGCTACACCTGCAACGTGAAGGCTCGATCCTGCGAGAA |
| | | GGAAGTGGTCTCTGCCCAGCCTGCCACCTTCCTGGCCCGTAGCC |
| | | CTCACGTGGGTGTGAAGGACGTGGAGTGTGGGGAAGGACACTT |
| | | CTGCCATGATAACCAGACCTGCTGCCGAGACAACCGACAGGGC |
| | | TGGGCCTGCTGTCCCTACCGCCAGGGCGTCTGTTGTGCTGATCG |
| | | GCGCCACTGCTGTCCTGCTGGCTTCCGCTGCGCAGCCAGGGGTA |
| | | CCAAGTGTTTGCGCAGGGAGGCCCCGCGCTGGGACGCCCCTTTG |
| | | AGGGACCCAGCCTTGAGACAGCTGCTGTGAAATAAAGGAAATT |
| | | TATTTTCATTGCAATAGTGTGTTGGAATTTTTTGTGTCTCTCACT |
| | | GCAGAGGAACCCCTAGTGATGGAGTTGGCCACTCCCTCTCTGCG |
| | | CGCTCGCTCGCTCACTGAGGCCGGGCGACCAAAGGTCGCCCGA |
| | | CGCCCGGGCTTTGCCCGGGCGGCCTCAGTGAGCGAGCGAGCGC |
| | | GCAG |
| 151 | Progranulin Payload | GTGGGTTACATCGAACTGGATCTCAACAGCGGTAAGATCCTTGA |
| | Plasmid | GAGTTTTCGCCCCGAAGAACGTTTTCCAATGATGAGCACTTTTA |
| | (STX_C0231) | AAGTTCTGCTATGTGGCGCGGTATTATCCCGTATTGACGCCGGG |
| | | CAAGAGCAACTCGGTCGCCGCATACACTATTCTCAGAATGACTT |
| | | GGTTGAGTACTCACCAGTCACAGAAAAGCATCTTACGGATGGC |
| | | ATGACAGTAAGAGAATTATGCAGTGCTGCCATAACCATGAGTG |
| | | ATAACACTGCGGCCAACTTACTTCTGACAACGATCGGAGGACC |
| | | GAAGGAGCTAACCGCTTTTTTGCACAACATGGGGGATCATGTA |
| | | ACTCGCCTTGATCGTTGGGAACCGGAGCTGAATGAAGCCATAC |
| | | AACGTTGCGCAAACTATTAACTGGCGAACTACTTACTCTAGCTT |
| | | CCCGGCAACAATTAATAGACTGGATGGAGGCGGATAAAGTTGC |
| | | AGGACCACTTCTGCGCTCGGCCCTTCCGGCTGGCTGGTTTATTG |
| | | CTGATAAATCTGGAGCCGGTGAGCGTGGGTCTCGCGGTATCATT |
| | | GCAGCACTGGGGCCAGATGGTAAGCCCTCCCGTATCGTAGTTAT |
| | | CTACACGACGGGGAGTCAGGCAACTATGGATGAACGAAATAGA |
| | | CAGATCGCTGAGATAGGTGCCTCACTGATTAAGCATTGGTAACT |
| | | GTCAGACCAAGTTTACTCATATATACTTTAGATTGATTTAAAAC |
| | | TTCATTTTTAATTTAAAAGGATCTAGGTGAAGATCCTTTTTGATA |
| | | ATCTCATGACCAAAATCCCTTAACGTGAGTTTTCGTTCCACTGA |
| | | GCGTCAGACCCCGTAGAAAAGATCAAAGGATCTTCTTGAGATC |
| | | CTTTTTTTCTGCGCGTAATCTGCTGCTTGCAAACAAAAAAACCA |
| | | CCGCTACCAGCGGTGGTTTGTTTGCCGGATCAAGAGCTACCAAC |
| | | TCTTTTTCCGAAGGTAACTGGCTTCAGCAGAGCGCAGATACCAA |
| | | ATACTGTTCTTCTAGTGTAGCCGTAGTTAGGCCACCACTTCAAG |
| | | AACTCTGTAGCACCGCCTACATACCTCGCTCTGCTAATCCTGTT |
| | | ACCAGTGGCTGCTGCCAGTGGCGATAAGTCGTGTCTTACCGGGT |
| | | TGGACTCAAGACGATAGTTACCGGATAAGGCGCAGCGGTCGGG |
| | | CTGAACGGGGGGTTCGTGCACACAGCCCAGCTTGGAGCGAACG |
| | | ACCTACACCGAACTGAGATACCTACAGCGTGAGCTATGAGAAA |
| | | GCGCCACGCTTCCCGAAGGGAGAAAGGCGGACAGGTATCCGGT |
| | | AAGCGGCAGGGTCGGAACAGGAGAGCGCACGAGGGAGCTTCC |
| | | AGGGGGAAACGCCTGGTATCTTTATAGTCCTGTCGGGTTTCGCC |
| | | ACCTCTGACTTGAGCGTCGATTTTTGTGATGCTCGTCAGGGGGG |
| | | CGGAGCCTATGGAAAAACGCCAGCAACGCGGCCTTTTTACGGT |
| | | TCCTGGCCTTTTGCTGGCCTTTTGCTCACATGTTCTTTCCTGCGT |
| | | TATCCCCTGATTCTGTGGATAACCGTATTACCGCCTTTGAGTGA |
| | | GCTGATACCGCTCGCCGCAGCCGAACGACCGAGCGCAGCGAGT |
| | | CAGTGAGCGAGGAAGCGGAAGAGCGCCCAATACGCAAACCGC |
| | | CTCTCCCCGCGCGTTGGCCGATTCATTAATGCAGCTGGCACGAC |
| | | AGGTTTCCCGACTGGAAAGCGGGCAGTGAGCGCAACGCAATTA |
| | | ATGTGAGTTAGCTCACTCATTAGGCACCCCAGGCTTTACACTTT |
| | | ATGCTTCCGGCTCGTATGTTGTGTGGAATTGTGAGCGGATAACA |
| | | ATTTCACACAGGAAACAGCTATGACCATGATTACGCCAAGCGC |
| | | GCCCGCCGGGTAACTCACGGGGTATCCATGTCCATTTCTGCGGC |
| | | ATCCAGCCAGGATACCCGTCCTCGCTGACGTAATATCCCAGCGC |
| | | CGCACCGCTGTCATTAATCTGCACACCGGCACGGCAGTTCCGGC |
| | | TGTCGCCGGTATTGTTCGGGTTGCTGATGCGCTTCGGGCTGACC |
| | | ATCCGGAACTGTGTCCGGAAAAGCCGCGACGAACTGGTATCCC |
| | | AGGTGGCCTGAACGAACAGTTCACCGTTAAAGGCGTGCATGGC |
| | | CACACCTTCCCGAATCATCATGGTAAACGTGCGTTTTCGCTCAA |
| | | CGTCAATGCAGCAGCAGTCATCCTCGGCAAACTCTTTCCATGCC |
| | | GCTTCAACCTCGCGGGAAAAGGCACGGGCTTCTTCCTCCCCGAT |
| | | GCCCAGATAGCGCCAGCTTGGGCGATGACTGAGCCGGAAAAAA |
| | | GACCCGACGATATGATCCTGATGCAGCTAGATTAACCCTAGAA |
| | | AGATAGTCTGCGTAAAATTGACGCATGCATTCTTGAAATATTGC |
| | | TCTCTCTTTCTAAATAGCGCGAATCCGTCGCTGTGCATTTAGGA |
| | | CATCTCAGTCGCCGCTTGGAGCTCCCGTGAGGCGTGCTTGTCAA |
| | | TGCGGTAAGTGTCACTGATTTTGAACTATAACGACCGCGTGAGT |
| | | CAAAATGACGCATGATTATCTTTTACGTGACTTTTAAGATTTAA |
| | | CTCATACGATAATTATATTGTTATTTCATGTTCTACTTACGTGAT |
| | | AACTTATTATATATATATTTTCTTGTTATAGATATCAGCCATACC |
| | | AATGGGCCCTAAAAAAGGAATCCAGTCAATTCCGGGGCTAAAC |
| | | CTGGCTGCCACTGTTTCTTTAGGGACTTCGTTCCTGTGAGGACA |
| | | GGAAACCCTGCAACCTGGTTGACCAGAGCAAGGGATATAAGAA |
| | | CCCAGTTTGGGAACACTGCTGTTTAAATATTAAACAGGGATGTC |
| | | TTGTCCCCCGGAAGGGAGACCAGATGAGCTTCTGATAGACTAA |
| | | TTGACATCATTTGAGTCAATTCCAGACATGATAAGATACATTGA |
| | | TGAGTTTGGACAAACCACAACTAGAATGCAGTGAAAAAAATGC |
| | | TTTATTTGTGAAATTTGTGATGCTATTGCTTTATTTGTAACCATT |
| | | ATAAGCTGCAATAAACAAGTTTAGTTAACGCATGATACAAAGG |
| | | CATTAAAGCAGCGTATCCACATAGCGTAAAAGGAGCAACATAG |
| | | TTAAGAATACCAGTCAATCTTTCACAAATTTTGTAATCCAGAGG |
| | | TTGATTTCAGGCACCGGGCTTGCGGGTCATGCACTCAGTTTGGA |
| | | AGGTTGTCGACTCTCATCAGATCCAGCTCTCGTTCAAAAATTTC |
| | | GTCAATTGGCAGCATCTGGCCGTCCACGGTCATAAACTTATGAT |
| | | CTTTAGTAGCGCGGATGAGGGATCCGTCCTCGAGACAGTATTCA |
| | | AAAACTTCCTGCTCTCCTCTGTCGTGCCATTGAGCCACGGGCTG |
| | | TGTGTAGATGTTCCCATTATTATCGACTGAATAGACGGTACATT |
| | | CAATCCTCTTCTCCACGATTTTGCCGATAGGAAGCAATCCGTAC |
| | | TCGACAGTCAGGATCTCGGTCTCGTATGAAAGGCACATACCAC |
| | | AAGGACTTACCACTCGAATACTTCTATCTACTTCGTCAGAATAA |
| | | GGGTGTCTAACAGCTACAATCGTGTCAAAATCCTTTTGTCCATT |
| | | CGAAACTGCACTACCAATCGCAATGGCTTCTGCACAAACAGTTA |
| | | CTCGTCCTATATACGCTTCAATATGTACTGCCGAAATGATTTCTC |
| | | CTGTTTTCGTACGAATTGCCGCTCCCACATGATGTTTATTATCCT |
| | | CATAAAGCATTGTAATCTTCTCTGTCGCTACTTCTACTAATTCTA |
| | | GATCCTGTTGAGAAATGTTAAATGTTTTCATGGTGGCGATATCG |
| | | TAGGCGCCGGTCACAGCTTGGATCTGTAACGGCGCAGAACAGA |
| | | AAACGAAACAAAGACGTAGAGTTGAGCAAGCAGGGTCAGGCA |
| | | AAGCGTGGAGAGCCGGCTGAGTCTAGGTAGGCTCCAAGGGAGC |
| | | GCCGGACAAAGGCCCGGTCTCGACCTGAGCTTTAAACTTACCTA |
| | | GACGGCGGACGCAGTTCAGGAGGCACCACAGGCGGGAGGCGG |
| | | CAGAACGCGACTCAACCGGCGTGGATGGCGGCCTCAGGTAGGG |
| | | CGGCGGGCGCGTGAAGGAGAGATGCGAGCCCCTCGAAGCTTCA |
| | | GCTGTGTTCTGGCGGCAAACCCGTTGCGAAAAAGAACGTTCAC |
| | | GGCGACTACTGCACTTATATACGGTTCTCCCCCACCCTCGGGAA |
| | | AAAGGCGGAGCCAGTACACGACATCACTTTCCCAGTTTACCCCG |
| | | CGCCACCTTCTCTAGGCACCCGTTCAATTGCCGACCCCTCCCCC |
| | | CAACTTCTCGGGGACTGTGGGCGATGTGCGCTCTGCCCACTGAC |
| | | GGGCACCGGAGCGATCGCAGATCCTTGCGGCCGCGGATCCGAT |
| | | TTAAATTCGAATTCGCTAGCTCTAGTGCGGTGTGAAATACCGCA |
| | | CAGATGCGTAAGGAGAAAATACCGCATCAGGCGCCATTCGCCA |
| | | TTCAGGCTGCGCAACTGTTGGGAAGGGCGATCGGTGCGGGCCT |
| | | CTTCGCTATTACGCCAGCTGGCGAAAGGGGGATGTGCTGCAAG |
| | | GCGATTAAGTTGGGTAACGCCAGGGTTTTCCCAGTCACGACGTT |
| | | GTAAAACGACGGCCAGTGAATTCTCTGCGCGCTCGCTCGCTCAC |
| | | TGAGGCCGGGCGACCAAAGGTCGCCCGACGCCCGGGCTTTGCC |
| | | CGGGCGGCCTCAGTGAGCGAGCGAGCGCGCAGAGGATCCAATG |
| | | TAGTCTTATGCAATACTCTTGTAGTCTTGCAACATGGTAACGAT |
| | | GAGTTAGCAACATGCCTTACAAGGAGAGAAAAAGCACCGTGCA |
| | | TGCCGATTGGTGGAAGTAAGGTGGTACGATCGTGCCTTATTAGG |
| | | AAGGCAACAGACGGGTCTGACATGGATTGGACGAACCACTGAA |
| | | TTCCGCATTGCAGAGATATTGTATTTAAGTGCCTAGCTCGATAC |
| | | AATAAACGCCATTTGACCATTCACCACATTGGTGTGCACCGCTA |
| | | GCGCCGCCACCATGTGGACCCTGGTGAGCTGGGTGGCCTTAAC |
| | | AGCAGGGCTGGTGGCTGGAACGCGGTGCCCAGATGGTCAGTTC |
| | | TGCCCTGTGGCCTGCTGCCTGGACCCCGGAGGAGCCAGCTACA |
| | | GCTGCTGCCGTCCCCTTCTGGACAAATGGCCCACAACACTGAGC |
| | | AGGCATCTGGGTGGCCCCTGCCAGGTTGATGCCCACTGCTCTGC |
| | | CGGCCACTCCTGCATCTTTACCGTCTCAGGGACTTCCAGTTGCT |
| | | GCCCCTTCCCAGAGGCCGTGGCATGCGGGGATGGCCATCACTG |
| | | CTGCCCACGGGGCTTCCACTGCAGTGCAGACGGGCGATCCTGCT |
| | | TCCAAAGATCAGGTAACAACTCCGTGGGTGCCATCCAGTGCCCT |
| | | GATAGTCAGTTCGAATGCCCGGACTTCTCCACGTGCTGTGTTAT |
| | | GGTCGATGGCTCCTGGGGGTGCTGCCCCATGCCCCAGGCTTCCT |
| | | GCTGTGAAGACAGGGTGCACTGCTGTCCGCACGGTGCCTTCTGC |
| | | GACCTGGTTCACACCCGCTGCATCACACCCACGGGCACCCACCC |
| | | CCTGGCAAAGAAGCTCCCTGCCCAGAGGACTAACAGGGCAGTG |
| | | GCCTTGTCCAGCTCGGTCATGTGTCCGGACGCACGGTCCCGGTG |
| | | CCCTGATGGTTCTACCTGCTGTGAGCTGCCCAGTGGGAAGTATG |
| | | GCTGCTGCCCAATGCCCAACGCCACCTGCTGCTCCGATCACCTG |
| | | CACTGCTGCCCCCAAGACACTGTGTGTGACCTGATCCAGAGTAA |
| | | GTGCCTCTCCAAGGAGAACGCTACCACGGACCTCCTCACTAAGC |
| | | TGCCTGCGCACACAGTGGGGGATGTGAAATGTGACATGGAGGT |
| | | GAGCTGCCCAGATGGCTATACCTGCTGCCGTCTACAGTCGGGGG |
| | | CCTGGGGCTGCTGCCCTTTTACCCAGGTACCCAGGGGTGGCGGG |
| | | TGGGTGGGCTGAGCACAGTGTGGCAGGCAGCCGGGCCCCAGTG |
| | | CCCACCTGCCCTTCTTCATCTGCCCTAGGCTGTGTGCTGTGAGG |
| | | ACCACATACACTGCTGTCCCGCGGGGTTTACGTGTGACACGCAG |
| | | AAGGGTACCTGTGAACAGGGGCCCCACCAGGTGCCCTGGATGG |
| | | AGAAGGCCCCAGCTCACCTCAGCCTGCCAGACCCACAAGCCTT |
| | | GAAGAGAGATGTCCCCTGTGATAATGTCAGCAGCTGTCCCTCCT |
| | | CCGATACCTGCTGCCAACTCACGTCTGGGGAGTGGGGCTGCTGT |
| | | CCAATCCCAGAGGCTGTCTGCTGCTCGGACCACCAGCACTGCTG |
| | | CCCCCAGGGCTACACGTGTGTAGCTGAGGGGCAGTGTCAGCGA |
| | | GGAAGCGAGATCGTGGCTGGACTGGAGAAGATGCCTGCCCGCC |
| | | GGGCTTCCTTATCCCACCCCAGAGACATCGGCTGTGACCAGCAC |
| | | ACCAGCTGCCCGGTGGGGCAGACCTGCTGCCCGAGCCTGGGTG |
| | | GGAGCTGGGCCTGCTGCCAGTTGCCCCATGCTGTGTGCTGCGAG |
| | | GATCGCCAGCACTGCTGCCCGGCTGGCTACACCTGCAACGTGA |
| | | AGGCTCGATCCTGCGAGAAGGAAGTGGTCTCTGCCCAGCCTGC |
| | | CACCTTCCTGGCCCGTAGCCCTCACGTGGGTGTGAAGGACGTGG |
| | | AGTGTGGGGAAGGACACTTCTGCCATGATAACCAGACCTGCTG |
| | | CCGAGACAACCGACAGGGCTGGGCCTGCTGTCCCTACCGCCAG |
| | | GGCGTCTGTTGTGCTGATCGGCGCCACTGCTGTCCTGCTGGCTT |
| | | CCGCTGCGCAGCCAGGGGTACCAAGTGTTTGCGCAGGGAGGCC |
| | | CCGCGCTGGGACGCCCCTTTGAGGGACCCAGCCTTGAGACAGC |
| | | TGCTGTGAAATAAAGGAAATTTATTTTCATTGCAATAGTGTGTT |
| | | GGAATTTTTTGTGTCTCTCACTGCAGAGGAACCCCTAGTGATGG |
| | | AGTTGGCCACTCCCTCTCTGCGCGCTCGCTCGCTCACTGAGGCC |
| | | GGGCGACCAAAGGTCGCCCGACGCCCGGGCTTTGCCCGGGCGG |
| | | CCTCAGTGAGCGAGCGAGCGCGCAGAGAGGGAGTGGCCAAAG |
| | | ATAAGCAGGAAAGAAATAAAAGTTTTGTTACTTTATAGAAGAA |
| | | ATTTTGAGTTTTTGTTTTTTTTAATAAATAAATAAACATAAATAA |
| | | ATTGTTTGTTGAATTTATTATTAGTATGTAAGTGTAAATATAATA |
| | | AAACTTAATATCTATTCAAATTAATAAATAAACCTCGATATACA |
| | | GACCGATAAAACACATGCGTCAATTTTACACATGATTATCTTTA |
| | | ACGTACGTCACAATATGATTATCTTTCTAGGGTTAATCTAGCTG |
| | | CGTGTTCTGCAGCGTGTCGAGCATCTTCATCTGCTCCATCACGC |
| | | TGTAAAACACATTTGCACCGCGAGTCTGCCCGTCCTCCACGGGT |
| | | TCAAAAACGTGAATGAACGAGGCGCGCTCACTGGCCGTCGTTTT |
| | | ACAACGTCGTGACTGGGAAAACCCTGGCGTTACCCAACTTAATC |
| | | GCCTTGCAGCACATCCCCCTTTCGCCAGCTGGCGTAATAGCGAA |
| | | GAGGCCCGCACCGATCGCCCTTCCCAACAGTTGCGCAGCCTGA |
| | | ATGGCGAATGGGACGCGCCCTGTAGCGGCGCATTAAGCGCGGC |
| | | GGGTGTGGTGGTTACGCGCAGCGTGACCGCTACACTTGCCAGC |
| | | GCCCTAGCGCCCGCTCCTTTCGCTTTCTTCCCTTCCTTTCTCGCC |
| | | ACGTTCGCCGGCTTTCCCCGTCAAGCTCTAAATCGGGGGCTCCC |
| | | TTTAGGGTTCCGATTTAGTGCTTTACGGCACCTCGACCCCAAAA |
| | | AACTTGATTAGGGTGATGGTTCACGTAGTGGGCCATCGCCCTGA |
| | | TAGACGGTTTTTCGCCCTTTGACGTTGGAGTCCACGTTCTTTAAT |
| | | AGTGGACTCTTGTTCCAAACTGGAACAACACTCAACCCTATCTC |
| | | GGTCTATTCTTTTGATTTATAAGGGATTTTGCCGATTTCGGCCTA |
| | | TTGGTTAAAAAATGAGCTGATTTAACAAAAATTTAACGCGAATT |
| | | TTAACAAAATATTAACGCTTACAATTTAGGTGGCACTTTTCGGG |
| | | GAAATGTGCGCGGAACCCCTATTTGTTTATTTTTCTAAATACATT |
| | | CAAATATGTATCCGCTCATGAGACAATAACCCTGATAAATGCTT |
| | | CAATAATATTGAAAAAGGAAGAGTATGAGTATTCAACATTTCC |
| | | GTGTCGCCCTTATTCCCTTTTTTGCGGCATTTTGCCTTCCTGTTTT |
| | | TGCTCACCCAGAAACGCTGGTGAAAGTAAAAGATGCTGAAGAT |
| | | CAGTTGGGTGCACGA |
| 152 | Progranulin payload | TTGGCCACTCCCTCTCTGCGCGCTCGCTCGCTCACTGAGGCCGC |
| | (ITR to ITR) (of | CCGGGCAAAGCCCGGGCGTCGGGCGACCTTTGGTCGCCCGGCC |
| | SEQ ID NO: 153) | TCAGTGAGCGAGCGAGCGCGCAGAGAGGGAGTGGCCAACTCCA |
| | | TCACTAGGGGTTCCTCTGCAGTGAGAGACACAAAAAATTCCAA |
| | | CACACTATTGCAATGAAAATAAATTTCCTTTATTTCACAGCAGC |
| | | TGTCTCAAGGCTGGGTCCCTCAAAGGGGCGTCCCAGCGCGGGG |
| | | CCTCCCTGCGCAAACACTTGGTACCCCTGGCTGCGCAGCGGAAG |
| | | CCAGCAGGACAGCAGTGGCGCCGATCAGCACAACAGACGCCCT |
| | | GGCGGTAGGGACAGCAGGCCCAGCCCTGTCGGTTGTCTCGGCA |
| | | GCAGGTCTGGTTATCATGGCAGAAGTGTCCTTCCCCACACTCCA |
| | | CGTCCTTCACACCCACGTGAGGGCTACGGGCCAGGAAGGTGGC |
| | | AGGCTGGGCAGAGACCACTTCCTTCTCGCAGGATCGAGCCTTCA |
| | | CGTTGCAGGTGTAGCCAGCCGGGCAGCAGTGCTGGCGATCCTC |
| | | GCAGCACACAGCATGGGGCAACTGGCAGCAGGCCCAGCTCCCA |
| | | CCCAGGCTCGGGCAGCAGGTCTGCCCCACCGGGCAGCTGGTGT |
| | | GCTGGTCACAGCCGATGTCTCTGGGGTGGGATAAGGAAGCCCG |
| | | GCGGGCAGGCATCTTCTCCAGTCCAGCCACGATCTCGCTTCCTC |
| | | GCTGACACTGCCCCTCAGCTACACACGTGTAGCCCTGGGGGCA |
| | | GCAGTGCTGGTGGTCCGAGCAGCAGACAGCCTCTGGGATTGGA |
| | | CAGCAGCCCCACTCCCCAGACGTGAGTTGGCAGCAGGTATCGG |
| | | AGGAGGGACAGCTGCTGACATTATCACAGGGGACATCTCTCTTC |
| | | AAGGCTTGTGGGTCTGGCAGGCTGAGGTGAGCTGGGGCCTTCTC |
| | | CATCCAGGGCACCTGGTGGGGCCCCTGTTCACAGGTACCCTTCT |
| | | GCGTGTCACACGTAAACCCCGCGGGACAGCAGTGTATGTGGTC |
| | | CTCACAGCACACAGCCTGGGTAAAAGGGCAGCAGCCCCAGGCC |
| | | CCCGACTGTAGACGGCAGCAGGTATAGCCATCTGGGCAGCTCA |
| | | CCTCCATGTCACATTTCACATCCCCCACTGTGTGCGCAGGCAGC |
| | | TTAGTGAGGAGGTCCGTGGTAGCGTTCTCCTTGGAGAGGCACTT |
| | | ACTCTGGATCAGGTCACACACAGTGTCTTGGGGGCAGCAGTGC |
| | | AGGTGATCGGAGCAGCAGGTGGCGTTGGGCATTGGGCAGCAGC |
| | | CATACTTCCCACTGGGCAGCTCACAGCAGGTAGAACCATCAGG |
| | | GCACCGGGACCGTGCGTCCGGACACATGACCGAGCTGGACAAG |
| | | GCCACTGCCCTGTTAGTCCTCTGGGCAGGGAGCTTCTTTGCCAG |
| | | GGGGTGGGTGCCCGTGGGTGTGATGCAGCGGGTGTGAACCAGG |
| | | TCGCAGAAGGCACCGTGCGGACAGCAGTGCACCCTGTCTTCAC |
| | | AGCAGGAAGCCTGGGGCATGGGGCAGCACCCCCAGGAGCCATC |
| | | GACCATAACACAGCACGTGGAGAAGTCCGGGCATTCGAACTGA |
| | | CTATCAGGGCACTGGATGGCACCCACGGAGTTGTTACCTGATCT |
| | | TTGGAAGCAGGATCGCCCGTCTGCACTGCAGTGGAAGCCCCGT |
| | | GGGCAGCAGTGATGGCCATCCCCGCATGCCACGGCCTCTGGGA |
| | | AGGGGCAGCAACTGGAAGTCCCTGAGACGGTAAAGATGCAGGA |
| | | GTGGCCGGCAGAGCAGTGGGCATCAACCTGGCAGGGGCCACCC |
| | | AGATGCCTGCTCAGTGTTGTGGGCCATTTGTCCAGAAGGGGACG |
| | | GCAGCAGCTGTAGCTGGCTCCTCCGGGGTCCAGGCAGCAGGCC |
| | | ACAGGGCAGAACTGACCATCTGGGCACCGCGTTCCAGCCACCA |
| | | GCCCTGCTGTTAAGGCCACCCAGCTCACCAGGGTCCACATGGTG |
| | | GCGGCGCTAGCGCCTAGAAGTAAAGGCAACATCCACTGAGGAG |
| | | CAGTTCTTTGATTTGCACCACCACCGGATCCGGGACCTGAAATA |
| | | AAAGACAAAAAGACTAAACTTACGCTAGCCGGTGCACACCAAT |
| | | GTGGTGAATGGTCAAATGGCGTTTATTGTATCGAGCTAGGCACT |
| | | TAAATACAATATCTCTGCAATGCGGAATTCAGTGGTTCGTCCAA |
| | | TCCATGTCAGACCCGTCTGTTGCCTTCCTAATAAGGCACGATCG |
| | | TACCACCTTACTTCCACCAATCGGCATGCACGGTGCTTTTTCTCT |
| | | CCTTGTAAGGCATGTTGCTAACTCATCGTTACCATGTTGCAAGA |
| | | CTACAAGAGTATTGCATAAGACTACATTGGATCCTCTGCGCGCT |
| | | CGCTCGCTCACTGAGGCCGCCCGGGCAAAGCCCGGGCGTCGGG |
| | | CGACCTTTGGTCGCCCGGCCTCAGTGAGCGAGCGAGCGCGCAG |
| | | AGA |
| 153 | Progranulin Payload | GCGCCTAGAAGTAAAGGCAACATCCACTGAGGAGCAGTTCTTT |
| | Plasmid | GATTTGCACCACCACCGGATCCGGGACCTGAAATAAAAGACAA |
| | (STX_C0232) | AAAGACTAAACTTACGCTAGCCGGTGCACACCAATGTGGTGAA |
| | | TGGTCAAATGGCGTTTATTGTATCGAGCTAGGCACTTAAATACA |
| | | ATATCTCTGCAATGCGGAATTCAGTGGTTCGTCCAATCCATGTC |
| | | AGACCCGTCTGTTGCCTTCCTAATAAGGCACGATCGTACCACCT |
| | | TACTTCCACCAATCGGCATGCACGGTGCTTTTTCTCTCCTTGTAA |
| | | GGCATGTTGCTAACTCATCGTTACCATGTTGCAAGACTACAAGA |
| | | GTATTGCATAAGACTACATTGGATCCTCTGCGCGCTCGCTCGCT |
| | | CACTGAGGCCGCCCGGGCAAAGCCCGGGCGTCGGGCGACCTTT |
| | | GGTCGCCCGGCCTCAGTGAGCGAGCGAGCGCGCAGAGAATTCA |
| | | CTGGCCGTCGTTTTACAACGTCGTGACTGGGAAAACCCTGGCGT |
| | | TACCCAACTTAATCGCCTTGCAGCACATCCCCCTTTCGCCAGCT |
| | | GGCGTAATAGCGAAGAGGCCCGCACCGATCGCCCTTCCCAACA |
| | | GTTGCGCAGCCTGAATGGCGAATGGCGCCTGATGCGGTATTTTC |
| | | TCCTTACGCATCTGTGCGGTATTTCACACCGCACTAGAGCTAGC |
| | | GAATTCGAATTTAAATCGGATCCGCGGCCGCAAGGATCTGCGA |
| | | TCGCTCCGGTGCCCGTCAGTGGGCAGAGCGCACATCGCCCACA |
| | | GTCCCCGAGAAGTTGGGGGGAGGGGTCGGCAATTGAACGGGTG |
| | | CCTAGAGAAGGTGGCGCGGGGTAAACTGGGAAAGTGATGTCGT |
| | | GTACTGGCTCCGCCTTTTTCCCGAGGGTGGGGGAGAACCGTATA |
| | | TAAGTGCAGTAGTCGCCGTGAACGTTCTTTTTCGCAACGGGTTT |
| | | GCCGCCAGAACACAGCTGAAGCTTCGAGGGGCTCGCATCTCTC |
| | | CTTCACGCGCCCGCCGCCCTACCTGAGGCCGCCATCCACGCCGG |
| | | TTGAGTCGCGTTCTGCCGCCTCCCGCCTGTGGTGCCTCCTGAAC |
| | | TGCGTCCGCCGTCTAGGTAAGTTTAAAGCTCAGGTCGAGACCGG |
| | | GCCTTTGTCCGGCGCTCCCTTGGAGCCTACCTAGACTCAGCCGG |
| | | CTCTCCACGCTTTGCCTGACCCTGCTTGCTCAACTCTACGTCTTT |
| | | GTTTCGTTTTCTGTTCTGCGCCGTTACAGATCCAAGCTGTGACCG |
| | | GCGCCTACGATATCGCCACCATGAAAACATTTAACATTTCTCAA |
| | | CAGGATCTAGAATTAGTAGAAGTAGCGACAGAGAAGATTACAA |
| | | TGCTTTATGAGGATAATAAACATCATGTGGGAGCGGCAATTCGT |
| | | ACGAAAACAGGAGAAATCATTTCGGCAGTACATATTGAAGCGT |
| | | ATATAGGACGAGTAACTGTTTGTGCAGAAGCCATTGCGATTGGT |
| | | AGTGCAGTTTCGAATGGACAAAAGGATTTTGACACGATTGTAGC |
| | | TGTTAGACACCCTTATTCTGACGAAGTAGATAGAAGTATTCGAG |
| | | TGGTAAGTCCTTGTGGTATGTGCCTTTCATACGAGACCGAGATC |
| | | CTGACTGTCGAGTACGGATTGCTTCCTATCGGCAAAATCGTGGA |
| | | GAAGAGGATTGAATGTACCGTCTATTCAGTCGATAATAATGGG |
| | | AACATCTACACACAGCCCGTGGCTCAATGGCACGACAGAGGAG |
| | | AGCAGGAAGTTTTTGAATACTGTCTCGAGGACGGATCCCTCATC |
| | | CGCGCTACTAAAGATCATAAGTTTATGACCGTGGACGGCCAGA |
| | | TGCTGCCAATTGACGAAATTTTTGAACGAGAGCTGGATCTGATG |
| | | AGAGTCGACAACCTTCCAAACTGAGTGCATGACCCGCAAGCCC |
| | | GGTGCCTGAAATCAACCTCTGGATTACAAAATTTGTGAAAGATT |
| | | GACTGGTATTCTTAACTATGTTGCTCCTTTTACGCTATGTGGATA |
| | | CGCTGCTTTAATGCCTTTGTATCATGCGTTAACTAAACTTGTTTA |
| | | TTGCAGCTTATAATGGTTACAAATAAAGCAATAGCATCACAAAT |
| | | TTCACAAATAAAGCATTTTTTTCACTGCATTCTAGTTGTGGTTTG |
| | | TCCAAACTCATCAATGTATCTTATCATGTCTGGAATTGACTCAA |
| | | ATGATGTCAATTAGTCTATCAGAAGCTCATCTGGTCTCCCTTCC |
| | | GGGGGACAAGACATCCCTGTTTAATATTTAAACAGCAGTGTTCC |
| | | CAAACTGGGTTCTTATATCCCTTGCTCTGGTCAACCAGGTTGCA |
| | | GGGTTTCCTGTCCTCACAGGAACGAAGTCCCTAAAGAAACAGT |
| | | GGCAGCCAGGTTTAGCCCCGGAATTGACTGGATTCCTTTTTTAG |
| | | GGCCCATTGGTATGGCTGATATCTATAACAAGAAAATATATATA |
| | | TAATAAGTTATCACGTAAGTAGAACATGAAATAACAATATAATT |
| | | ATCGTATGAGTTAAATCTTAAAAGTCACGTAAAAGATAATCATG |
| | | CGTCATTTTGACTCACGCGGTCGTTATAGTTCAAAATCAGTGAC |
| | | ACTTACCGCATTGACAAGCACGCCTCACGGGAGCTCCAAGCGG |
| | | CGACTGAGATGTCCTAAATGCACAGCGACGGATTCGCGCTATTT |
| | | AGAAAGAGAGAGCAATATTTCAAGAATGCATGCGTCAATTTTA |
| | | CGCAGACTATCTTTCTAGGGTTAATCTAGCTGCATCAGGATCAT |
| | | ATCGTCGGGTCTTTTTTCCGGCTCAGTCATCGCCCAAGCTGGCG |
| | | CTATCTGGGCATCGGGGAGGAAGAAGCCCGTGCCTTTTCCCGCG |
| | | AGGTTGAAGCGGCATGGAAAGAGTTTGCCGAGGATGACTGCTG |
| | | CTGCATTGACGTTGAGCGAAAACGCACGTTTACCATGATGATTC |
| | | GGGAAGGTGTGGCCATGCACGCCTTTAACGGTGAACTGTTCGTT |
| | | CAGGCCACCTGGGATACCAGTTCGTCGCGGCTTTTCCGGACACA |
| | | GTTCCGGATGGTCAGCCCGAAGCGCATCAGCAACCCGAACAAT |
| | | ACCGGCGACAGCCGGAACTGCCGTGCCGGTGTGCAGATTAATG |
| | | ACAGCGGTGCGGCGCTGGGATATTACGTCAGCGAGGACGGGTA |
| | | TCCTGGCTGGATGCCGCAGAAATGGACATGGATACCCCGTGAG |
| | | TTACCCGGCGGGCGCGCTTGGCGTAATCATGGTCATAGCTGTTT |
| | | CCTGTGTGAAATTGTTATCCGCTCACAATTCCACACAACATACG |
| | | AGCCGGAAGCATAAAGTGTAAAGCCTGGGGTGCCTAATGAGTG |
| | | AGCTAACTCACATTAATTGCGTTGCGCTCACTGCCCGCTTTCCA |
| | | GTCGGGAAACCTGTCGTGCCAGCTGCATTAATGAATCGGCCAA |
| | | CGCGCGGGGAGAGGCGGTTTGCGTATTGGGCGCTCTTCCGCTTC |
| | | CTCGCTCACTGACTCGCTGCGCTCGGTCGTTCGGCTGCGGCGAG |
| | | CGGTATCAGCTCACTCAAAGGCGGTAATACGGTTATCCACAGA |
| | | ATCAGGGGATAACGCAGGAAAGAACATGTGAGCAAAAGGCCA |
| | | GCAAAAGGCCAGGAACCGTAAAAAGGCCGCGTTGCTGGCGTTT |
| | | TTCCATAGGCTCCGCCCCCCTGACGAGCATCACAAAAATCGACG |
| | | CTCAAGTCAGAGGTGGCGAAACCCGACAGGACTATAAAGATAC |
| | | CAGGCGTTTCCCCCTGGAAGCTCCCTCGTGCGCTCTCCTGTTCC |
| | | GACCCTGCCGCTTACCGGATACCTGTCCGCCTTTCTCCCTTCGG |
| | | GAAGCGTGGCGCTTTCTCATAGCTCACGCTGTAGGTATCTCAGT |
| | | TCGGTGTAGGTCGTTCGCTCCAAGCTGGGCTGTGTGCACGAACC |
| | | CCCCGTTCAGCCCGACCGCTGCGCCTTATCCGGTAACTATCGTC |
| | | TTGAGTCCAACCCGGTAAGACACGACTTATCGCCACTGGCAGC |
| | | AGCCACTGGTAACAGGATTAGCAGAGCGAGGTATGTAGGCGGT |
| | | GCTACAGAGTTCTTGAAGTGGTGGCCTAACTACGGCTACACTAG |
| | | AAGAACAGTATTTGGTATCTGCGCTCTGCTGAAGCCAGTTACCT |
| | | TCGGAAAAAGAGTTGGTAGCTCTTGATCCGGCAAACAAACCAC |
| | | CGCTGGTAGCGGTGGTTTTTTTGTTTGCAAGCAGCAGATTACGC |
| | | GCAGAAAAAAAGGATCTCAAGAAGATCCTTTGATCTTTTCTACG |
| | | GGGTCTGACGCTCAGTGGAACGAAAACTCACGTTAAGGGATTT |
| | | TGGTCATGAGATTATCAAAAAGGATCTTCACCTAGATCCTTTTA |
| | | AATTAAAAATGAAGTTTTAAATCAATCTAAAGTATATATGAGTA |
| | | AACTTGGTCTGACAGTTACCAATGCTTAATCAGTGAGGCACCTA |
| | | TCTCAGCGATCTGTCTATTTCGTTCATCCATAGTTGCCTGACTCC |
| | | CCGTCGTGTAGATAACTACGATACGGGAGGGCTTACCATCTGGC |
| | | CCCAGTGCTGCAATGATACCGCGAGACCCACGCTCACCGGCTCC |
| | | AGATTTATCAGCAATAAACCAGCCAGCCGGAAGGGCCGAGCGC |
| | | AGAAGTGGTCCTGCAACTTTATCCGCCTCCATCCAGTCTATTAA |
| | | TTGTTGCCGGGAAGCTAGAGTAAGTAGTTCGCCAGTTAATAGTT |
| | | TGCGCAACGTTGTTGCCATTGCTACAGGCATCGTGGTGTCACGC |
| | | TCGTCGTTTGGTATGGCTTCATTCAGCTCCGGTTCCCAACGATC |
| | | AAGGCGAGTTACATGATCCCCCATGTTGTGCAAAAAAGCGGTT |
| | | AGCTCCTTCGGTCCTCCGATCGTTGTCAGAAGTAAGTTGGCCGC |
| | | AGTGTTATCACTCATGGTTATGGCAGCACTGCATAATTCTCTTA |
| | | CTGTCATGCCATCCGTAAGATGCTTTTCTGTGACTGGTGAGTAC |
| | | TCAACCAAGTCATTCTGAGAATAGTGTATGCGGCGACCGAGTTG |
| | | CTCTTGCCCGGCGTCAATACGGGATAATACCGCGCCACATAGCA |
| | | GAACTTTAAAAGTGCTCATCATTGGAAAACGTTCTTCGGGGCGA |
| | | AAACTCTCAAGGATCTTACCGCTGTTGAGATCCAGTTCGATGTA |
| | | ACCCACTCGTGCACCCAACTGATCTTCAGCATCTTTTACTTTCAC |
| | | CAGCGTTTCTGGGTGAGCAAAAACAGGAAGGCAAAATGCCGCA |
| | | AAAAAGGGAATAAGGGCGACACGGAAATGTTGAATACTCATAC |
| | | TCTTCCTTTTTCAATATTATTGAAGCATTTATCAGGGTTATTGTC |
| | | TCATGAGCGGATACATATTTGAATGTATTTAGAAAAATAAACAA |
| | | ATAGGGGTTCCGCGCACATTTCCCCGAAAAGTGCCACCTAAATT |
| | | GTAAGCGTTAATATTTTGTTAAAATTCGCGTTAAATTTTTGTTAA |
| | | ATCAGCTCATTTTTTAACCAATAGGCCGAAATCGGCAAAATCCC |
| | | TTATAAATCAAAAGAATAGACCGAGATAGGGTTGAGTGTTGTTC |
| | | CAGTTTGGAACAAGAGTCCACTATTAAAGAACGTGGACTCCAA |
| | | CGTCAAAGGGCGAAAAACCGTCTATCAGGGCGATGGCCCACTA |
| | | CGTGAACCATCACCCTAATCAAGTTTTTTGGGGTCGAGGTGCCG |
| | | TAAAGCACTAAATCGGAACCCTAAAGGGAGCCCCCGATTTAGA |
| | | GCTTGACGGGGAAAGCCGGCGAACGTGGCGAGAAAGGAAGGG |
| | | AAGAAAGCGAAAGGAGCGGGCGCTAGGGCGCTGGCAAGTGTA |
| | | GCGGTCACGCTGCGCGTAACCACCACACCCGCCGCGCTTAATGC |
| | | GCCGCTACAGGGCGCGTCCCATTCGCCATTCAGGCTGCGCAACT |
| | | GTTGGGAAGGGCGATCGGTGCGGGCCTCTTCGCTATTACGCCAG |
| | | CTGGCGAAAGGGGGATGTGCTGCAAGGCGATTAAGTTGGGTAA |
| | | CGCCAGGGTTTTCCCAGTCACGACGTTGTAAAACGACGGCCAGT |
| | | GAGCGCGCCTCGTTCATTCACGTTTTTGAACCCGTGGAGGACGG |
| | | GCAGACTCGCGGTGCAAATGTGTTTTACAGCGTGATGGAGCAG |
| | | ATGAAGATGCTCGACACGCTGCAGAACACGCAGCTAGATTAAC |
| | | CCTAGAAAGATAATCATATTGTGACGTACGTTAAAGATAATCAT |
| | | GTGTAAAATTGACGCATGTGTTTTATCGGTCTGTATATCGAGGT |
| | | TTATTTATTAATTTGAATAGATATTAAGTTTTATTATATTTACAC |
| | | TTACATACTAATAATAAATTCAACAAACAATTTATTTATGTTTAT |
| | | TTATTTATTAAAAAAAACAAAAACTCAAAATTTCTTCTATAAAG |
| | | TAACAAAACTTTTATTTCTTTCCTGCGTTATCTTTGGCCACTCCC |
| | | TCTCTGCGCGCTCGCTCGCTCACTGAGGCCGCCCGGGCAAAGCC |
| | | CGGGCGTCGGGCGACCTTTGGTCGCCCGGCCTCAGTGAGCGAG |
| | | CGAGCGCGCAGAGAGGGAGTGGCCAACTCCATCACTAGGGGTT |
| | | CCTCTGCAGTGAGAGACACAAAAAATTCCAACACACTATTGCA |
| | | ATGAAAATAAATTTCCTTTATTTCACAGCAGCTGTCTCAAGGCT |
| | | GGGTCCCTCAAAGGGGCGTCCCAGCGCGGGGCCTCCCTGCGCA |
| | | AACACTTGGTACCCCTGGCTGCGCAGCGGAAGCCAGCAGGACA |
| | | GCAGTGGCGCCGATCAGCACAACAGACGCCCTGGCGGTAGGGA |
| | | CAGCAGGCCCAGCCCTGTCGGTTGTCTCGGCAGCAGGTCTGGTT |
| | | ATCATGGCAGAAGTGTCCTTCCCCACACTCCACGTCCTTCACAC |
| | | CCACGTGAGGGCTACGGGCCAGGAAGGTGGCAGGCTGGGCAGA |
| | | GACCACTTCCTTCTCGCAGGATCGAGCCTTCACGTTGCAGGTGT |
| | | AGCCAGCCGGGCAGCAGTGCTGGCGATCCTCGCAGCACACAGC |
| | | ATGGGGCAACTGGCAGCAGGCCCAGCTCCCACCCAGGCTCGGG |
| | | CAGCAGGTCTGCCCCACCGGGCAGCTGGTGTGCTGGTCACAGC |
| | | CGATGTCTCTGGGGTGGGATAAGGAAGCCCGGCGGGCAGGCAT |
| | | CTTCTCCAGTCCAGCCACGATCTCGCTTCCTCGCTGACACTGCC |
| | | CCTCAGCTACACACGTGTAGCCCTGGGGGCAGCAGTGCTGGTG |
| | | GTCCGAGCAGCAGACAGCCTCTGGGATTGGACAGCAGCCCCAC |
| | | TCCCCAGACGTGAGTTGGCAGCAGGTATCGGAGGAGGGACAGC |
| | | TGCTGACATTATCACAGGGGACATCTCTCTTCAAGGCTTGTGGG |
| | | TCTGGCAGGCTGAGGTGAGCTGGGGCCTTCTCCATCCAGGGCAC |
| | | CTGGTGGGGCCCCTGTTCACAGGTACCCTTCTGCGTGTCACACG |
| | | TAAACCCCGCGGGACAGCAGTGTATGTGGTCCTCACAGCACAC |
| | | AGCCTGGGTAAAAGGGCAGCAGCCCCAGGCCCCCGACTGTAGA |
| | | CGGCAGCAGGTATAGCCATCTGGGCAGCTCACCTCCATGTCACA |
| | | TTTCACATCCCCCACTGTGTGCGCAGGCAGCTTAGTGAGGAGGT |
| | | CCGTGGTAGCGTTCTCCTTGGAGAGGCACTTACTCTGGATCAGG |
| | | TCACACACAGTGTCTTGGGGGCAGCAGTGCAGGTGATCGGAGC |
| | | AGCAGGTGGCGTTGGGCATTGGGCAGCAGCCATACTTCCCACT |
| | | GGGCAGCTCACAGCAGGTAGAACCATCAGGGCACCGGGACCGT |
| | | GCGTCCGGACACATGACCGAGCTGGACAAGGCCACTGCCCTGT |
| | | TAGTCCTCTGGGCAGGGAGCTTCTTTGCCAGGGGGGGGTGCCC |
| | | GTGGGTGTGATGCAGCGGGTGTGAACCAGGTCGCAGAAGGCAC |
| | | CGTGCGGACAGCAGTGCACCCTGTCTTCACAGCAGGAAGCCTG |
| | | GGGCATGGGGCAGCACCCCCAGGAGCCATCGACCATAACACAG |
| | | CACGTGGAGAAGTCCGGGCATTCGAACTGACTATCAGGGCACT |
| | | GGATGGCACCCACGGAGTTGTTACCTGATCTTTGGAAGCAGGAT |
| | | CGCCCGTCTGCACTGCAGTGGAAGCCCCGTGGGCAGCAGTGAT |
| | | GGCCATCCCCGCATGCCACGGCCTCTGGGAAGGGGCAGCAACT |
| | | GGAAGTCCCTGAGACGGTAAAGATGCAGGAGTGGCCGGCAGAG |
| | | CAGTGGGCATCAACCTGGCAGGGGCCACCCAGATGCCTGCTCA |
| | | GTGTTGTGGGCCATTTGTCCAGAAGGGGACGGCAGCAGCTGTA |
| | | GCTGGCTCCTCCGGGGTCCAGGCAGCAGGCCACAGGGCAGAAC |
| | | TGACCATCTGGGCACCGCGTTCCAGCCACCAGCCCTGCTGTTAA |
| | | GGCCACCCAGCTCACCAGGGTCCACATGGTGGCGGCGCTA |

### Exemplary Construct 1/Rep/Cap Features

| Name | type | location |
|---|---|---|
| Rep68 CDS2 | misc_feature | 4012 . . . 4036 |
| Rabbit beta globin | intronmisc_feature | 878 . . . 1230 |
| Rep40 CDS2 | misc_feature | 4012 . . . 4036 |
| 3′SS | misc_feature | 2760 . . . 2806 |
| VP1 | misc_feature | 3987 . . . 6194 |
| EGFP | misc_feature | 1312 . . . 2030 |
| VP3 | misc_feature | 4593 . . . 6194 |
| loxP | misc_feature | 1231 . . . 1264 |
| Rep52 | misc_feature | 848 . . . 877 |
| Rep40 CDS1 | misc_feature | 848 . . . 877 |
| Rep78 | misc_feature | 176 . . . 877 |
| Rep68 CDS1 | misc_feature | 176 . . . 877 |
| 5 prime terminus of | RNA1misc_feature | 142 . . . 151 |
| TATA box P5 | misc_feature | 110 . . . 115 |
| TATA box p19 | misc_feature | 698 . . . 705 |
| 5 prime terminus of | RNA2misc_feature | 728 . . . 737 |
| 5 prime terminus of | RNA3misc_feature | 3637 . . . 3644 |
| TATA box of P40 | misc_feature | 3606 . . . 3614 |
| polyA sequence | misc_feature | 6208 . . . 6213 |
| Rep52 | misc_feature | 2807 . . . 3970 |
| Rep40 CDS1 | misc_feature | 2807 . . . 3690 |
| Rep78 | misc_feature | 2807 . . . 3970 |
| Rep68 CDS1 | misc_feature | 2807 . . . 3690 |
| AAV 3′UTR | misc_feature | 6195 . . . 6318 |
| 3′SS | misc_feature | 1265 . . . 1311 |
| Rabbit beta globin | polyAmisc_feature | 2032 . . . 2552 |
| rabbit beta globin | intronmisc_feature | 2587 . . . 2806 |
| VP (CAP) proteins | misc_feature | 3987 . . . 6194 |
| pCRII Topo | misc_feature | 6319 . . . 9837 |
| M13-fwd | primer_bind | rev: 6415 . . . 6432 |
| M13-rev | primer_bind | 9707 . . . 9727 |
| T7 | primer_bind | rev: 6389 . . . 6416 |
| SP6 | primer_bind | 9733 . . . 9758 |
| ColE1 origin | rep_origin | 8657 . . . 9339 |
| LacO | misc_binding | 9679 . . . 9701 |
| LoxP | misc_recomb | rev: 1231 . . . 1264 |
| LoxP | misc_recomb | rev: 2553 . . . 2586 |
| Kan/neoR | CDS | 7223 . . . 8014 |

### EQUIVALENTS AND INCORPORATION BY REFERENCE

All references cited herein are incorporated by reference to the same extent as if each individual publication, database entry (e.g., Genbank sequences or GeneID entries), patent application, or patent, was specifically and individually indicated to be incorporated by reference in its entirety, for all purposes. This statement of incorporation by reference is intended by Applicants, pursuant to 37 C.F.R. § 1.57 (b) (1), to relate to each and every individual publication, database entry (e.g., Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

While the invention has been particularly shown and described with reference to a preferred embodiment and various alternate embodiments, it will be understood by persons skilled in the relevant art that various changes in form and details can be made therein without departing from the spirit and scope of the invention.

What is claimed is:

1. A cell for inducibly producing recombinant adenovirus associated virus (rAAV) virions, the cell comprising:
(a) a first polynucleotide construct comprising from 5′ to 3′:
one or more promoters operably linked to a first sequence comprising a first part of an AAV Rep coding sequence;
a second sequence comprising a second part of the AAV Rep coding sequence, wherein the one or more promoters are not operably linked to the second sequence comprising the second part of the AAV Rep coding sequence,
wherein the first sequence and the second sequence are separated by (i) an excisable element that comprises a first recombination site and a second recombination site flanking a coding sequence encoding a stop signaling sequence, wherein the first recombination site and the second recombination site are oriented in the same direction or (ii) an inversible element that comprises a first recombination site and a second recombination site flanking a coding sequence encoding a stop signaling sequence, wherein the first recombination site and the second recombination site are oriented in opposite directions, and;
a third sequence comprising a sequence encoding one or more AAV capsid proteins, wherein the second sequence comprises a promoter that is operably linked to the third sequence; and
a first constitutive promoter operably linked to a sequence encoding a first selectable marker;
(b) a second polynucleotide construct comprising from 5′ to 3′:
an inducible promoter operably linked to a self-excising element; the self-excising element comprising a third recombination site and a fourth recombination site flanking a sequence encoding an inducible recombinase, wherein the third recombination site and the fourth recombination site are oriented in the same direction; a sequence encoding one or more AAV helper proteins, wherein the inducible promoter is not operably linked to the sequence encoding the one or more AAV helper proteins;
a second constitutive promoter operably linked to a sequence encoding an activator, wherein the cell constitutively expresses the activator and the activator is unable to activate the inducible promoter in absence of a triggering agent; and
a third constitutive promoter operably linked to a sequence encoding a second selectable marker, wherein the cell constitutively expresses the second selectable marker,
wherein in absence of activation of the inducible promoter, the cell expresses a fusion protein comprising a Rep protein encoded by the first part of the AAV Rep coding sequence that terminates at the stop signaling sequence, and does not express detectable levels of the inducible recombinase and the one or more AAV helper proteins; and
(c) a third polynucleotide construct comprising a promoter operably linked to a sequence encoding a payload and a third selectable marker, wherein the sequence encoding the payload is flanked by a 5′ AAV inverted terminal repeat (5′ ITR) and a 3′ AAV inverted terminal repeat (3′ ITR), and
wherein the payload is progranulin (PGRN).

2. The cell of claim 1, wherein the coding sequence encoding the stop signaling sequence further encodes a protein marker that comprises the stop signaling sequence.

3. The cell of claim 1 or 2, wherein the cell further comprises:
an adenovirus E1A protein and E1B protein, and the one or more AAV helper proteins expressed by the second polynucleotide construct are an adenovirus E2A protein and E4 protein,
or
an adenovirus E2A protein and E4 protein, and the one or more AAV helper proteins expressed by the second polynucleotide construct are an adenovirus E1A protein and EB protein.

4. The cell of any one of claims 1-3, further comprising a fourth polynucleotide construct comprising an inducible or constitutive promoter operably linked to a sequence encoding one or more helper proteins.

5. The cell of any one of claims 1-4, wherein the sequence coding for one or more AAV helper proteins comprises a bicistronic open reading frame encoding two AAV helper proteins.

6. The cell of claim 5, wherein the two AAV helper proteins are E2A and E4 or ELA and E1B.

7. The cell of claim 5 or 6, wherein the bicistronic open reading frame comprises an internal ribosome entry site (IRES) or a peptide 2A (P2A) sequence.

8. The cell of claim 7, wherein transcription of the AAV Rep coding sequences is driven by the P5 and P19 native AAV promoters, and transcription of the sequence encoding the one or more AAV capsid proteins is driven by the P40 native AAV promoter.

9. The cell of any one of claims 1-7, wherein transcription of the AAV Rep coding sequences is driven by an inducible promoter and transcription of the sequence encoding the one or more AAV capsid proteins is driven by an inducible promoter.

10. The cell of any one of claims 1-9, wherein the AAV capsid proteins comprise VP1, VP2, and VP3.

11. The cell of any one of claims 1-10, wherein the cell is a mammalian cell.

12. The cell of claim 11, wherein the mammalian cell is a HEK293 cell.

13. The cell of any one of claims 1-12, wherein the first polynucleotide construct, the second polynucleotide construct, the third polynucleotide construct, or any combination thereof are integrated into the nuclear genome of the cell.

14. The cell of any one of claims 1-13, wherein the inducible promoter in the second polynucleotide construct is selected from the group consisting of a tetracycline-inducible promoter, an ecdysone-inducible promoter, and a cumate-inducible promoter,

15. The cell of claim 14, wherein the triggering agent for inducing the tetracycline-inducible promoter is tetracycline or doxycycline.

16. The cell of any one of claims 1-15, wherein the inducible recombinase is fused to an estrogen response element (ER) and translocates to the nucleus in the presence of tamoxifen.

17. The cell of any one of claims 1-16, wherein the recombination sites in the first polynucleotide construct and the second polynucleotide construct are lox sites and the recombinase is a cre recombinase or wherein the recombination sites in the first polynucleotide construct and the second polynucleotide are flippase recognition target (FRT) sites and the recombinase is a flippase (Flp) recombinase.

18. The cell of any one of claims 1-17, wherein upon expression of the inducible recombinase,
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein; and
recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins.

19. The cell of any one of claims 1-18, wherein the second polynucleotide construct further comprises an insert comprising a sequence encoding VA-RNA; or optionally, wherein a fifth construct comprises an insert comprising a sequence encoding VA-RNA.

20. The cell of claim 19, wherein the VA-RNA is wild-type VA-RNA or VA-RNA comprising one or more mutations in the VA-RNA internal promoter.

21. The cell of claim 19 or 20, wherein the insert comprises:
a first part of a second constitutive promoter and a second part of a second constitutive promoter separated by a second excisable element comprising a fifth recombination site and a sixth recombination site flanking a stuffer sequence, wherein the fifth and sixth recombination sites are oriented in the same direction, and
a VA-RNA coding sequence, wherein excision of the second excisable element by the inducible recombinase generates a functional complete second constitutive promoter operably linked to the VA-RNA coding sequence to allow expression of the VA-RNA.

22. The cell of claim 21, wherein
the first part of the second constitutive promoter comprises a distal sequence element (DSE) of an RNA polymerase III promoter, and the second part of the second constitutive promoter comprises a proximal sequence element (PSE) of an RNA polymerase III promoter,
or
the first part of the second constitutive promoter comprises a distal sequence element (DSE) of a U6 promoter, and the second part of the second constitutive promoter comprises a proximal sequence element (PSE) of a U6 promoter,
or
the first part of the second constitutive promoter comprises a distal sequence element (DSE) of a U7 promoter, and the second part of the second constitutive promoter comprises a proximal sequence element (PSE) of a U7 promoter.

23. The cell of any one of claims 1-22, wherein the first polynucleotide construct further comprises:
(i) a first spacer segment and a second spacer segment flanking the excisable element, wherein the first part of the AAV Rep coding sequence and the second part of the AAV Rep coding sequence are separated by the first spacer segment, the excisable element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site; or
(ii) a first spacer segment and a second spacer segment flanking the inversible element, wherein the first part of the AAV Rep coding sequence and the second part of the AAV Rep coding sequence are separated by the first spacer segment, the inversible element, and the second spacer segment, wherein the first spacer segment comprises a first intron and the second spacer segment comprises a second intron, wherein the first polynucleotide construct further comprises a 5′ splice site at the 5′ end of the first spacer segment, a first 3′ splice site at the 3′ end of the second spacer segment, and a second 3′ splice site at the 3′ end of the first recombination site.

24. The cell of claim 23, wherein first part of the AAV Rep coding sequence comprises a p5 internal promoter and a p19 internal promoter, and the second part of the AAV Rep coding sequence comprises a p40 internal promoter.

25. The cell of claim 24, wherein the excisable spacer is inserted at an insertion site between the p19 internal promoter and the p40 internal promoter of the AAV Rep coding sequence.

26. The cell of claim 25, wherein the insertion site is between a CAG and a G, a CAG and an A, an AAG and a G, and an AAG and an A.

27. The cell of any one of claims 1-26, wherein the first polynucleotide construct comprises at least 70%, 75%, 80%, 85%, 90%, 95%, or 100% sequence identity to SEQ ID NO: 136.

28. The cell of any one of claims 1-27, wherein the second polynucleotide construct comprises at least 70%, 75%, 80%, 85%, 90%, 95%, or 100% sequence identity to SEQ ID NO: 11.

29. The cell of any one of claims 1-28, wherein the third polynucleotide construct comprises at least 70%, 75%, 80%, 85%, 90%, 95%, or 100% sequence identity to SEQ ID NO: 146.

30. The cell of any one of claims 1-28, wherein the third polynucleotide construct comprises at least 70%, 75%, 80%, 85%, 90%, 95%, or 100% sequence identity to SEQ ID NO: 148.

31. The cell of any one of claims 1-28, wherein the third polynucleotide construct comprises at least 70%, 75%, 80%, 85%, 90%, 95%, or 100% sequence identity to SEQ ID NO: 150.

32. The cell of any one of claims 1-28, wherein the third polynucleotide construct comprises at least 70%, 75%, 80%, 85%, 90%, 95%, or 100% sequence identity to SEQ ID NO: 152.

33. The cell of any one of claims 1-29, wherein the sequence encoding the payload flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to SEQ ID NO: 146.

34. The cell of any one of claim 1-28 or 30, wherein the sequence encoding the payload flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to SEQ ID NO: 148.

35. The cell of any one of claim 1-28 or 31, wherein the sequence encoding the payload flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to SEQ ID NO: 150.

36. The cell of any one of claim 1-28 or 32, wherein the sequence encoding the payload flanked by a 5′ AAV inverted terminal repeat (5′ ITR) and a 3′ AAV inverted terminal repeat (3′ ITR) comprises at least 70%, 75%, 80%, 85%, 90%, 95%, or 100% sequence identity to SEQ ID NO: 152.

37. The cell any one of claim 1-36, wherein the third polynucleotide construct further comprises a spacer between the 5′ ITR and the sequence encoding the third selectable marker or a spacer between the sequence encoding the third selectable marker and the 3′ ITR, or a combination thereof.

38. The cell of claim 37, wherein the spacer ranges in length from 500 base pairs to 5000 base pairs.

39. The method of any one of claims 1-38, wherein the first polynucleotide construct, the second polynucleotide construct, the third polynucleotide construct, the fourth polynucleotide construct, the fifth polynucleotide construct, or any combination thereof are integrated into the nuclear genome of the cell.

40. A vector system for inducibly producing recombinant adenovirus associated virus (rAAV) virions, the vector system comprising:
(a) a first polynucleotide construct according to any one of claims 1-39;
(b) a second polynucleotide construct according to any one of claims 1-39; and
(c) a third polynucleotide construct according to any one of claims 1-39;
optionally further comprising (d) a fourth polynucleotide construct according to any one of claims 4-39; and further optionally comprising a fifth polynucleotide construct according to any one of claims 19-39.

41. A method of generating a cell line for inducibly producing recombinant AAV (rAAV) virions comprising a payload, the method comprising:
introducing into a cell a second polynucleotide construct according to any one of claims 1-39;
selecting for cells expressing the second selectable marker;
introducing into a cell of the cells expressing the second selectable marker a first polynucleotide construct according to any one of claims 1-39;
selecting for cells expressing the first selectable marker;
introducing into a cell of the cells expressing the second selectable marker and the first selectable marker a third polynucleotide construct according to any one of claims 1-39; and
selecting for cells expressing the third selectable marker; thereby generating the cell line inducibly producing recombinant AAV (rAAV) virions comprising a payload.

42. The method of claim 41, further comprising contacting a cell from the cell line for inducibly producing recombinant AAV (rAAV) virions comprising a payload with the triggering agent, wherein in the presence of the triggering agent, the activator activates the inducible promoter resulting in expression of the recombinase, wherein
(i) recombination between the first recombination site and the second recombination site in the first polynucleotide construct results in excision of the excisable element, and the first part of the AAV Rep coding sequence and the second part of the AAV Rep coding sequence are joined to form a complete AAV Rep coding sequence, or
(ii) recombination between the first recombination site and the second recombination site in the first polynucleotide construct results in inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein,
wherein the one or more promoters are operably linked to the complete AAV Rep coding sequence to allow expression of an AAV Rep protein and an AAV Cap protein; and
recombination between the third recombination site and the fourth recombination site in the second polynucleotide construct results in excision of the self-excising element comprising the sequence encoding the inducible recombinase, wherein the inducible promoter becomes operably linked to the sequence encoding the one or more AAV helper proteins to allow expression of the one or more AAV helper proteins; thereby inducibly producing recombinant AAV (rAAV) virions comprising a payload.

43. The method of claim 41 or 42, wherein the cell is a mammalian cell.

44. The method of claim 43, wherein the mammalian cell is a HEK293 cell.

45. The method of any one of claims 40-44, wherein the first polynucleotide construct, the second polynucleotide construct, the third polynucleotide construct, the fourth polynucleotide construct, the fifth polynucleotide construct, or any combination thereof are integrated into the nuclear genome of the cell.

46. The method of any one of claims 40-45, wherein the first polynucleotide construct is in a plasmid comprising at least 70%, 75%, 80%, 85%, 90%, 95%, or 100% sequence identity to SEQ ID NO: 32.

47. The method of any one of claims 40-46, wherein the second polynucleotide construct is in a plasmid comprising at least 70%, 75%, 80%, 85%, 90%, 95%, or 100% sequence identity to SEQ ID NO: 30.

48. The method of any one of claims 40-47, wherein the third polynucleotide construct is in a plasmid comprising at least 70%, 75%, 80%, 85%, 90%, 95%, or 100% sequence identity to SEQ ID NO: 147.

49. The method of any one of claims 40-47, wherein the third polynucleotide construct is in a plasmid comprising at least 70%, 75%, 80%, 85%, 90%, 95%, or 100% sequence identity to SEQ ID NO: 149.

50. The method of any one of claims 40-47, wherein the third polynucleotide construct is in a plasmid comprising at least 70%, 75%, 80%, 85%, 90%, 95%, or 100% sequence identity to SEQ ID NO: 151.

51. The method of any one of claims 40-47, wherein the third polynucleotide construct is in a plasmid comprising at least 70%, 75%, 80%, 85%, 90%, 95%, or 100% sequence identity to SEQ ID NO: 153.

52. A method for generating a recombinant adenovirus associated virus (rAAV) virion comprising a sequence encoding progranulin, the method comprising contacting the cell according to any one of claims 1-39 with the triggering agent,
wherein in the presence of the triggering agent, the activator activates the inducible promoter of the second polynucleotide construct resulting in expression of the recombinase, wherein recombination between the first recombination site and the second recombination site in the first polynucleotide construct by the recombinase results in excision of the excisable element or inversion of the inversible element, and the first part of the AAV Rep coding sequence and the second part of the AAV Rep coding sequence are joined to form a complete AAV Rep coding sequence, wherein the one or more promoters are operably linked to the complete AAV Rep coding sequence to allow expression of an AAV Rep protein and an AAV Cap protein, and recombination between the third recombination site and the fourth recombination site in the second polynucleotide construct by the recombinase results in excision of the self-excising element comprising the sequence encoding the inducible recombinase, wherein the inducible promoter becomes operably linked to the sequence encoding the one or more AAV helper proteins to allow expression of the one or more AAV helper proteins,
wherein the expression of the one or more AAV helper proteins results in expression of the one or more Rep proteins and the one or more capsid proteins, thereby generating an rAAV virion comprising the sequence encoding the payload of interest.


SEQUENCE LISTING (155 sequences)

SEQ ID NO: 1 - DNA, 353 bp, synthetic construct
  source 1..353: mol_type = other DNA
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaac

SEQ ID NO: 2 - DNA, 1356 bp, synthetic construct
  source 1..1356: mol_type = other DNA
ataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttat

SEQ ID NO: 3 - DNA, 220 bp, synthetic construct
  source 1..220: mol_type = other DNA
aattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacag

SEQ ID NO: 4 - DNA, 534 bp, synthetic construct
  source 1..534: mol_type = other DNA
atgagagggtcagatccggcagcgttgaagcgggctaggaacactgaggcagcaaggcgctctcgagcaaggaagttgcaacggatgaaacaattggaagacaaagttgaagagctgctttcaaaaaactaccaccttgaaaatgaagtcgcgaggctgaagaaattggtcggatctgctggcagcgcagcggggagcggtgagtttatggtcagacctctcaactgtattgtcgctgtctcacagaacatgggtatcggaaagaacggtgacttgccgtggccgccactgcggaatgagttcaaatactttcagcgcatgacgaccaccagcagtgtggagggtaagcaaaatcttgtcataatgggtcgcaagacttggttttctattccagagaaaaacagaccgcttaaagataggattaacatcgtgttgagccgggaactgaaagagccaccaaggggagcacattttttggctaagtccttggatgacgccctgcgactgatagagcaaccagaacttgcttagtaa

SEQ ID NO: 5 - DNA, 453 bp, synthetic construct
  source 1..453: mol_type = other DNA
atgcgcggttccgacccagcagctttgaaacgagcacgaaacacggaagcagcccgcaggagtcgagcgagaaaacttcagcggatgaagcagcttgaagataaagtcgaggaattgcttagcaagaattatcacctcgagaatgaagtggcgcgactgaaaaaacttgtaggttctgctgggagcgcagccggaagcggcgagttctcaaaagttgacatggtgtggatcgtgggtggaagttctgtctatcaagaggcgatgaatcagcctggccacctcagactgtttgttacaaggatcatgcaggagttcgagtctgacacgttttttccagagatcgacctggggaaatataaactcctcccagagtacccaggagtgcttagtgaggtccaagaagagaagggaatcaaatataaatttgaagtttacgaaaagaaggattagtaa

SEQ ID NO: 6 - DNA, 9837 bp, synthetic construct
  source 1..9837: mol_type = other DNA
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacataagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgccct

SEQ ID NO: 7 - DNA, 6367 bp, synthetic construct
  source 1..6367: mol_type = other DNA
ggcctccacggccactagtaacggccgccagtgtgctggaattcgccctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactaca

SEQ ID NO: 8 - DNA, 12385 bp, synthetic construct
  source 1..12385: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtaacggccgccagtgtgctggaattcgccctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataatgtatgctatacgaagttatcgggcccctctgctaaccatgttcatgccttcttctttttcctacagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataatgtatgctatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacataagggcgaattctgcagatatccatcacactggcggccgctcgagcatgcatctagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaggtacctcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 9 - DNA, 14766 bp, synthetic construct
  source 1..14766: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcataggtccagggttctcctccacgtctccagcctgcttcagcaggctgaagttagtagctccggatcctttacctccatcaccagcgccaccagtagagtatctggccacagccacctcgtgctgctcgacgtaggtctcatcgtcggcctccttgattctttccagtctgtggtccacgttgtagacgccgggcatcttgaggttcgtagcgggtttcttggatctgtatgtggtctcaaggttgcagatcaggtggcccccgcccacgagcttcagggccatgtcacatgcgccttccaggccgccgtcagcggggtacatcgtctcggtggaggcctcccagccgagtgttttcttctgcatcacagggccgttggctgggaagttcacccctctaaccttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgcccggagggtgcttgatgaaggttctgctgccgtacatgaagctggtagccaggatgtcgaaggcgaaggggagagggccgccctcgacgaccttgattctcatggtctgggtgccctcgtagggcttgccttcgccctcggatgtgcacttgaagtggtggttgttcacggtgccctccatgtacagcttcatgggcatgttctccttaatcagctcgctcacggtggcggcgaattccgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 10 - DNA, 13743 bp, synthetic construct
  source 1..13743: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggagctactaacttcagcctgctgaagcaggctggagacgtggaggagaaccctggacctatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 11 - DNA, 10347 bp, synthetic construct
  source 1..10347: mol_type = other DNA
gcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcataggtccagggttctcctccacgtctccagcctgcttcagcaggctgaagttagtagctccggatcctttacctccatcaccagcgccaccagtagagtatctggccacagccacctcgtgctgctcgacgtaggtctcatcgtcggcctccttgattctttccagtctgtggtccacgttgtagacgccgggcatcttgaggttcgtagcgggtttcttggatctgtatgtggtctcaaggttgcagatcaggtggcccccgcccacgagcttcagggccatgtcacatgcgccttccaggccgccgtcagcggggtacatcgtctcggtggaggcctcccagccgagtgttttcttctgcatcacagggccgttggctgggaagttcacccctctaaccttgacgttgtagatgaggcagccgtcctggaggctggtgtcctgggtagcggtcagcacgcccccgtcttcgtatgtggtgactctctcccatgtgaagccctcagggaaggactgcttaaagaagtcggggatgcccggagggtgcttgatgaaggttctgctgccgtacatgaagctggtagccaggatgtcgaaggcgaaggggagagggccgccctcgacgaccttgattctcatggtctgggtgccctcgtagggcttgccttcgccctcggatgtgcacttgaagtggtggttgttcacggtgccctccatgtacagcttcatgggcatgttctccttaatcagctcgctcacggtggcggcgaattccgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcc

SEQ ID NO: 12 - DNA, 9344 bp, synthetic construct
  source 1..9344: mol_type = other DNA
gcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggggatccggagctactaacttcagcctgctgaagcaggctggagacgtggaggagaaccctggacctatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatg

SEQ ID NO: 13 - DNA, 161 bp, synthetic construct
  source 1..161: mol_type = other DNA
gggcactcttccgtggtctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 14 - DNA, 151 bp, synthetic construct
  source 1..151: mol_type = other DNA
gggcactcttccgtggtctggtggataaattcgcaagggtatcatggcggacgaccggccggatccggccgtccgccgtgatccatgcggttaccgcccgcgtgtcgaacccaggtgtgcgacgtcagacaacgggggagcgctccttttt

SEQ ID NO: 15 - DNA, 161 bp, synthetic construct
  source 1..161: mol_type = other DNA
gggcactcttccgtgatctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 16 - DNA, 161 bp, synthetic construct
  source 1..161: mol_type = other DNA
gggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctccttttt

SEQ ID NO: 17 - DNA, 393 bp, synthetic construct
  source 1..393: mol_type = other DNA
cgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtggtctggtggataaattcgcaagggtatcatggcggacgaccggccggatccggccgtccgccgtgatccatgcggttaccgcccgcgtgtcgaacccaggtgtgcgacgtcagacaacgggg

SEQ ID NO: 18 - DNA, 417 bp, synthetic construct
  source 1..417: mol_type = other DNA
cgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccggggttcgaaccccggatccggccgtccgccgtgatccatgcggttaccgcccgcgtgtcgaacccaggtgtgcgacgtcagacaacgggggagcgctccttttt

SEQ ID NO: 19 - DNA, 428 bp, synthetic construct
  source 1..428: mol_type = other DNA
ttcactagaatcgatggagggcctatttcccatgattccttcatatttgcatatacgatacaaggctgttagagagataattagaattaatttgactgtaaacacaaagatattagtacaaaataataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctccttttt

SEQ ID NO: 20 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 21 - DNA, 744 bp, synthetic construct
  source 1..744: mol_type = other DNA
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccggg

SEQ ID NO: 22 - DNA, 379 bp, synthetic construct
  source 1..379: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 23 - DNA, 11051 bp, synthetic construct
  source 1..11051: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtcgacgggcactcttccgtggtctggtggataaattcgcaagggtatcatggcggacgaaccggtaccgaaccccggatccggccgtccgccgtgatccatgcggttaccgcccgcgtgtcgaacccaggtgtgcgacgtcagacaacgggggagcgctcctttttcatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 24 - DNA, 11051 bp, synthetic construct
  source 1..11051: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtcgacgggcactcttccgtggtctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttcatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 25 - DNA, 6770 bp, synthetic construct
  source 1..6770: mol_type = other DNA
ccgcggccgccaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaaacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagcaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgcctcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgggcgcg

SEQ ID NO: 26 - DNA, 6770 bp, synthetic construct
  source 1..6770: mol_type = other DNA
ccgcggccgccaactttgtatagaaaagttgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccaagtttgtacaaaaaagcaggctgccaccatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtagacccagctttcttgtacaaagtgggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccatggccagtcgggaagaggagcagcgcgaaaccacccccgagcgcggacgcggtgcggcgcgacgtccaccaaccatggaggacgtgtcgtccccgtcgccgtcgccgccgcctccccgcgcgcccccaaaaaagcggctgaggcggcgtctcgagtccgaggacgaagaagactcgtcacaagatgcgctggtgccgcgcacacccagcccgcggccatcgacctcgacggcggatttggccattgcgtccaaaaagaaaaagaagcgcccctctcccaagcccgagcgcccgccatccccagaggtgatcgtggacagcgaggaagaaagagaagatgtggcgctacaaatggtgggtttcagcaacccaccggtgctaatcaagcacggcaagggaggtaagcgcacggtgcggcggctgaatgaagacgacccagtggcgcggggtatgcggacgcaagaggaaaaggaagagtccagtgaagcggaaagtgaaagcacggtgataaacccgctgagcctgccgatcgtgtctgcgtgggagaagggcatggaggctgcgcgcgcgttgatggacaagtaccacgtggataacgatctaaaggcaaacttcaagctactgcctgaccaagtggaagctctggcggccgtatgcaagacctggctaaacgaggagcaccgcgggttgcagctgaccttcaccagcaacaagacctttgtgacgatgatggggcgattcctgcaggcgtacctgcagtcgtttgcagaggtaacctacaagcaccacgagcccacgggctgcgcgttgtggctgcaccgctgcgctgagatcgaaggcgagcttaagtgtctacacgggagcattatgataaataaggagcacgtgattgaaatggatgtgacgagcgaaaacgggcagcgcgcgctgaaggagcagtctagcaaggccaagatcgtgaagaaccggtggggccgaaatgtggtgcagatctccaacaccgacgcaaggtgctgcgtgcatgacgcggcctgtccggccaatcagttttccggcaagtcttgcggcatgttcttctctgaaggcgcaaaggctcaggtggcttttaagcagatcaaggctttcatgcaggcgctgtatcctaacgcccagaccgggcacggtcaccttctgatgccactacggtgcgagtgcaactcaaagcctgggcatgcaccctttttgggaaggcagctaccaaagttgactccgttcgccctgagcaacgcggaggacctggacgcggatctgatctccgacaagagcgtgctggccagcgtgcaccacccggcgctgatagtgttccagtgctgcaaccctgtgtatcgcaactcgcgcgcgcagggcggaggccccaactgcgacttcaagatatcggcgcccgacctgctaaacgcgttggtgatggtgcgcagcctgtggagtgaaaacttcaccgagctgccgcggatggttgtgcctgagtttaagtggagcactaaacaccagtatcgcaacgtgtccctgccagtggcgcatagcgatgcgcggcagaacccctttgatttttaacaactttattatacatagttgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtagcggccgcggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctctcttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcggcgcgcctcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgggcgcg

SEQ ID NO: 27 - DNA, 14227 bp, synthetic construct
  source 1..14227: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttaggttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 28 - DNA, 14058 bp, synthetic construct
  source 1..14058: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtcaaccctcccacacgtaaccagatgggagaagctctcgtattccaacagctgtaggttgaccgtctgaatccttcactatggcctttatacctggatgcagatccaacaggacttgtctacatctaccacaaggtgacaagatcccgcgattctcgttaccaatagcaacgatacaagtcagattccctgcggcggcggcagcggcggttcccagtactacgagttctgcgcaggggccacccgtaaaatgatacacatttaccccagtaaaaatccgcccatcggatgacaaggctgcactagccacggaatagtcgtcagatataggtatggaattgattgtggcggtagccctctctatcagcgtggactcctcctgagagaggggttttgccatggtggcggcttaagggttcgatcctctagagtccggaggctggatcggtcccggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcattgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 29 - DNA, 14067 bp, synthetic construct
  source 1..14067: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggtctagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgtagggagttgatttcagactatgcaccagattgttttgtgttaatagaaatgaatggcaagttagtcaaaactacgattgaagaactcattccactcaaatatacccgaaattaaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagggcccattggtatggcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 30 - DNA, 13923 bp, synthetic construct
  source 1..13923: mol_type = other DNA
tggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatgattccttcatatttgcatatacgatacaaggctgttagagagataattagaattaatttgactgtaaacacaaagatattagtacaaaataataacttcgtataatgtatgctatacgaagttatcagacatgataagatacattgatgagtttggacaaaccacaactagaatgcagtgaaaaaaatgctttatttgtgaaatttgtgatgctattgctttatttgtaaccattataagctgcaataaacaaggtacctcaagcgccgggttttcgcgtcatgcaccacgtccgtggttcaagcgccgggttttcgcgtcatgcaccacgtccgtgggccctcgggtacttcaacgtcagcagtaactgtaaatccgagccgttcatagaagggcaaattccttggcgctgacgtttcaagaaaggctggcactccggctcgttctgcggcttctactccgggcaataccaccgcggaaccaaggccctttccctgatgatcggggctaacgcccacagtagcgaggaaccaagctggttctttagggcggtgaggggcgaggagtccttccatttgttgctgagccgcgagacgagagccactaagctcagccattcggggaccaatttctgcaaatacagccccggcctcaacgctctccggagtcgtccacactgccactgcagccccgtcgtcggcgacccaaactttaccgatgtccaatcctaccctggtcaaaaaaagttcttgcaattctgtaacccgttcaatatgtctatcaggatcaactgtgtggcgtgtagcgggataatccgcgaaagcggcagccaatgttctcacggccctagggacgtcgtctcgagttgccagtctgacagtaggtttatattctgtcatggtggcggcgaattctcttctatggaggtcaaaacagcgtggatggcgtctccaggcgatctgacggttcactaaacgagctctgcttatataaacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatactagttcttgggaaaagcgctcccctacccataacttcgtataatgtatgctatacgaagttattttgcagttttaaaattatgttttaaaatggactatcatatgcttaccgtaacttgaaagtatttcgatttcttggctttatatatcttgtggaaaggacgaaacaccgggcactcttccgtgatctggtggataaattcgcaagggtatcatggcggacgaccgggattcgaaccccggatccggccgtccgccgtgatccatgcggttaccgcccgcgtgtcgaacccaggtgtgcgacgtcagacaacgggggagcgctcctttttgggcccat

SEQ ID NO: 31 - DNA, 14372 bp, synthetic construct
  source 1..14372: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatgagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaggcctccacggccactagtccatagagcccaccgcatccccagcatgcctgctattgtcttcccaatcctcccccttgctgtcctgccccaccccaccccctagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcatgagacgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatgcactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgtcagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtctctgtctcaaaaggaggtaggcgatccctactgtacggagtgcgccgagacaaccgagatcgtgttggtcgtagtgtcatgccaaatggaacgccggacgtagtcatggttgtggccatattatcatcgtgtttttcaaaggaaaaccacgtccccgtggttcggggggcctagacgtttttttaacctcgactaaacacatgtaaagcatgtgcaccgaggccccagatcagatcccatacaatggggtaccttctgggcatccttcagccccttgttgaatacgcttgaggagagccatttgactctttccacaactatccaactcacaacgtggcactggggttgtgccgcctttgcaggtgtatcttatacacgtggcttttggccgcagaggcacctgtcgccaggtggggggttccgctgcctgcaaagggtcgctacagacgttgtttgtcttcaagaagcttccagaggaactgcttccttcacgacattcaacagaccttgcattcctttggcgagaggggaaagacccctaggaatgctcgtcaagaagacagggccaggtttccgggccctcacattgccaaaagacggcaatatggtggaaaataacatatagacaaacgcacaccggccttattccaagcggcttcggccagtaacgttaggggggggggagggagaggggcttaaaaatcaaaggggttctgccgcgcatcactatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcgcttgtcgtcatcgtctttgtagtcgggaggcggcggcgacggcgacggggacgacacgtcctccatggttggtggacgtcgcgccgcaccgcgtccgcgctcgggggtggtttcgcgctgctcctcttcccgactggccatggtggccgaggataacttcgtatatggtttcttatacgaagttatgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagagtcgcagatctgctacgtatcaagctgtggcagggaaaccctctgcctcccccgtgatgtaatacttttgcaaggaatgcgatgaagtagagcccgcagtggccaagtggctttggtccgtctcctccacggatgcccctccacggctagtgggcgcatgtaggcggtgggcgtccgccgcctccagcagcaggtcatagaggggcaccacgttcttgcacttcatgctgtacagatgctccatgcctttgttactcatgtgtcggatgtgggagaggatgaggaggagctgggccagccgctggtgctgctgctgcagggtcaggcctgccttggccatcaggtggatcaaagtgtctgtgatcttgtccaggactcggtggatatggtccttctcttccagagacttcagggtgctggacagaaatgtgtacactccagaattaagcaaaataatagatttgaggcacacaaactcctctccctgcagattcatcatgcggaaccgagatgatgtagccagcagcatgtcgaagatctccaccatgccctctacacattttccctggttcctgtccaagagcaagttaggagcaaacagtagcttcactgggtgctccatggagcgccagacgagaccaatcatcaggatctctagccaggcacattctagaaggtggacctgatcatggagggtcaaatccacaaagcctggcaccctcttcgcccagttgatcatgtgaaccagctccctgtctgccaggttggtcagtaagcccatcatcgaagcttcactgaagggtctggtaggatcatactcggaatagagtatggggggctcagcatccaacaaggcactgaccatctggtcggccgtcagggacaaggccaggctgttcttcttagagcgtttgatcatgagcgggcttggccaaaggttggcagctctcatgtctccagcagatggctcgagatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatcctaaaatacacaaacaattagaatcagtagtttaacacattatacacttaaaaattttatatttaccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagtgactcccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacggacttggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcttgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacatggtggctacgtaataacttcgtatatggtttcttatacgaagttatgcggccgctttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactcgcggccgcagagaaatgttctggcacctgcacttgcactggggacagcctattttgctagtttgttttgtttcgttttgttttgatggagagcgtatgttagtactatcgattcacacaaaaaaccaacacacagatgtaatgaaaataaagatattttattggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgctagcggatccgccgccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctcttctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtgaaccggtcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagagggcctatttcccatggtggtagaactagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaagagaattcgccgccaccatgacagaatataaacctactgtcagactggcaactcgagacgacgtccctagggccgtgagaacattggctgccgctttcgcggattatcccgctacacgccacacagttgatcctgatagacatattgaacgggttacagaattgcaagaactttttttgaccagggtaggattggacatcggtaaagtttgggtcgccgacgacggggctgcagtggcagtgtggacgactccggagagcgttgaggccggggctgtatttgcagaaattggtccccgaatggctgagcttagtggctctcgtctcgcggctcagcaacaaatggaaggactcctcgcccctcaccgccctaaagaaccagcttggttcctcgctactgtgggcgttagccccgatcatcagggaaagggccttggttccgcggtggtattgcccggagtagaagccgcagaacgagccggagtgccagcctttcttgaaacgtcagcgccaaggaatttgcccttctatgaacggctcggatttacagttactgctgacgttgaagtacccgagggcccacggacgtggtgcatgacgcgaaaacccggcgcttgagtttaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgactagagcttgcggaacccttagtttaaacgggcccttaattaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacggggcccattggtatggctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 32 - DNA, 11646 bp, synthetic construct
  source 1..11646: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaattaagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggcgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 33 - DNA, 8190 bp, synthetic construct
  source 1..8190: mol_type = other DNA
actcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggtctagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 34 - DNA, 4639 bp, synthetic construct
  source 1..4639: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctctgcgcgctcgctcgctcactgaggccgggcgaccaaaggtcgcccgacgcccgggctttgcccgggcggcctcagtgagcgagcgagcgcgcagaggatcctctacgccatattatccacagtccaacggccaggcggaggctagtaacaaggttatcctcggcatcctccgcaggtaccatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagacaccgggaccgatccagcctccggactctagaggatcgaacccttaagccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtgaaccggtcagacatgataagatacattgatgagtttggacaaaccacaactagaatgcagtgaaaaaaatgctttatttgtgaaatttgtgatgctattgctttatttgtaaccattataagctgcaataaacaagttggtaccctcacggaccagtgcaacatattcccaacatcccgttgcagcctatcattaaaccttggccggtcgcggctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 35 - DNA, 10898 bp, synthetic construct
  source 1..10898: mol_type = other DNA
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

SEQ ID NO: 36-39 - skipped (4 entries)

SEQ ID NO: 40 - protein, 248 aa, synthetic construct
MSRLDKSKVINSALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGGPADALDDFDLDMLPADALDDFDLDMLPADALDDFDLDMLPG

SEQ ID NO: 41 - protein, 335 aa, synthetic construct
MSRLDKSKVINSALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 42 - protein, 335 aa, synthetic construct
MSRLDKSKVINSALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 43 - protein, 335 aa, synthetic construct
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEEQEHQVAKEERETPTTDSMPPLLRQAIELFDRQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 44 - protein, 335 aa, synthetic construct
MSRLDKSKVINSALELLNEVGIEGLATRKLAQKLGVEQPTLYWHVKNKRALLDALAIEMLDRHHTHFCPLEGESWQDFLRNNAKSFRCALLSHRGGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 45 - protein, 335 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDRAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 46 - DNA, 450 bp, synthetic construct
  source 1..450: mol_type = other DNA
gaattcctcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagtttaccactccctatcagtgatagagaaaagtgaaagtcgagctcggtacccgggtcgagtaggcgtgtacggtgggaggcctatataagcagagctcgtttagtgaaccgtcagatcgcctggagacgccatccacgctgttttgacctccatagaagacaccgggaccgatccagcctccgcgg

SEQ ID NO: 47 - DNA, 450 bp, synthetic construct
  source 1..450: mol_type = other DNA
gaattcctcgacccgggtaccgagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagtaggcgtgtacggtgggaggcctatataagcagagctcgtttagtgaaccgtcagatcgcctggagacgccatccacgctgttttgacctccatagaagacaccgggaccgatccagcctccgcgg

SEQ ID NO: 48 - DNA, 398 bp, synthetic construct
  source 1..398: mol_type = other DNA
gagctcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagtggtaaactcgagatccggcgaattcgaacacgcagatgcagtcggggcggcgcggtccgaggtccacttcgcatattaaggtgacgcgtgtggcctcgaacaccgag

SEQ ID NO: 49 - protein, 207 aa, synthetic construct
MSRLDKSKVINSALELLNEVGIEGLTTRKLAQKLGVEQPTLYWHVKNKRALLDALAIEMLDRHHTHFCPLKGESWQDFLRNKAKSFRCALLSHRNGAKVHSDTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 50 - DNA, 345 bp, synthetic construct
  source 1..345: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagacgccatccacgctgtttccatagaaga

SEQ ID NO: 51 - DNA, 318 bp, synthetic construct
  source 1..318: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggaga

SEQ ID NO: 52 - DNA, 406 bp, synthetic construct
  source 1..406: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggaggtaatcaactaccaattccagctctcttttgacaactggtcttataccaactttccgtaccacttcctaccctcgtaagacaattgcaa

SEQ ID NO: 53 - DNA, 376 bp, synthetic construct
  source 1..376: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 54 - DNA, 407 bp, synthetic construct
  source 1..407: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagctaatcaactaccaattccagctctcttttgacaactggtcttataccaactttccgtaccacttcctaccctcctaagacaattgcaaa

SEQ ID NO: 55 - DNA, 462 bp, synthetic construct
  source 1..462: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagcttgcctatgttcttttggaatctatccaagtcttatgtaaatgcttatgtaaaccataatataaaagagtgctgattttttgagtaaacttgcaacagtcctaacattcttctctcgtgtgtttgtgtctgttcgccatcccgtctccgctcgtcacttatccttcacttttcagagggtccccccgcagatcccggtcaccctcaggtcgg

SEQ ID NO: 56 - DNA, 411 bp, synthetic construct
  source 1..411: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagcttccataatataaaagagtgctgattttttgagtaaacttgcaacagtcctaacattcttctctcgtgtgtttgtgtctgttcgccatcccgtctccgctcgtcacttatccttcacttttcagagggtccccccgcagatcccggtcaccctcaggtcgg

SEQ ID NO: 57 - DNA, 415 bp, synthetic construct
  source 1..415: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagcttccagggcgcctataaaagagtgctgattttttgagtaaacttgcaacagtcctaacattcttctctcgtgtgtttgtgtctgttcgccatcccgtctccgctcgtcacttatccttcacttttcagagggtccccccgcagatcccggtcaccctcaggtcgg

SEQ ID NO: 58 - DNA, 365 bp, synthetic construct
  source 1..365: mol_type = other DNA
tttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttgcttatgtaaaccagggcgcctataaaagagtgctgattttttgagtaaacttcaattccacaacacttttgtcttataccaactttccgtaccacttcctaccctcgtaaa

SEQ ID NO: 59 - DNA, 262 bp, synthetic construct
  source 1..262: mol_type = other DNA
gaattctttactccctatcagtgatagagaatgtatgaagagtttactccctatcagtgatagagaatgtatgcagactttactccctatcagtgatagagaatgtataaggagtttactccctatcagtgatagagaatgtatgaccagtttactccctatcagtgatagagaatgtatctacagtttactccctatcagtgatagagaatgtatatccagtttactccctatcagtgatagagaatgtataagctttagg

SEQ ID NO: 60 - DNA, 133 bp, synthetic construct
  source 1..133: mol_type = other DNA
catgtacagtgggcacctataaaagcagagctcatttagtgaactgtcagattgcctggagcaattccacaacacttttgtcttataccaactttccataccacttcctaccctcataaagtgcacaccatgg

SEQ ID NO: 61 - DNA, 150 bp, synthetic construct
  source 1..150: mol_type = other DNA
ccatggtgtgcactttatgagggtaggaagtggtatggaaagttggtataagacaaaagtgttgtggaattgctccaggcaatctgacagttcactaaatgagctctgcttttataggtgcccactgtacatgcctaagaattctttact

SEQ ID NO: 62 - DNA, 250 bp, synthetic construct
  source 1..250: mol_type = other DNA
ccctatcagtgatagagaatgtatgaagagtttactccctatcagtgatagagaatgtatgcagactttactccctatcagtgatagagaatgtataaggagtttactccctatcagtgatagagaatgtatgaccagtttactccctatcagtgatagagaatgtatctacagtttactccctatcagtgatagagaatgtatatccagtttactccctatcagtgatagagaatgtataagctttagg

SEQ ID NO: 63 - DNA, 128 bp, synthetic construct
  source 1..128: mol_type = other DNA
ggtaggcgtgtacggtgggaggcctatataagcagagctcgtttagtgaaccgtcagatcgcctggagacgccatccacgctgttttgacctccatagaagacaccgggaccgatccagcctccgcgg

SEQ ID NO: 64 - DNA, 126 bp, synthetic construct
  source 1..126: mol_type = other DNA
ggtaggcgtgtacggtgggcgcctataaaagcagagctcgtttagtgaaccgtcagatcgcctggagacgccatccacgctgttttgacctccatagaagacaccgggaccgatccagcctccgcg

SEQ ID NO: 65 - DNA, 66 bp, synthetic construct
  source 1..66: mol_type = other DNA
taggcgtgtacggtgggcgcctataaaagcagagctcgtttagtgaaccgtcagatcgcctggaga

SEQ ID NO: 66 - DNA, 89 bp, synthetic construct
  source 1..89: mol_type = other DNA
gtaatcaactaccaattccagctctcttttgacaactggtcttataccaactttccgtaccacttgcaaccctcgtaagacaattgcaa

SEQ ID NO: 67 - DNA, 58 bp, synthetic construct
  source 1..58: mol_type = other DNA
aattccacaacacttttgtcttataccaactttccgtaccacttcctaccctcgtaaa

SEQ ID NO: 68 - DNA, 210 bp, synthetic construct
  source 1..210: mol_type = other DNA
gcctatgttcttttggaatctatccaagtcttatgtaaatgcttatgtaaaccataatataaaagagtgctgattttttgagtaaacttgcaacagtcctaacattcttctctcgtgtgtttgtgtctgttcgccatcccgtctccgctcgtcacttatccttcacttttcagagggtccccccgcagatcccggtcaccctcaggtcgg

SEQ ID NO: 69 - DNA, 747 bp, synthetic construct
  source 1..747: mol_type = other DNA
atgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtaa

SEQ ID NO: 70 - DNA, 1008 bp, synthetic construct
  source 1..1008: mol_type = other DNA
atgtctagattagataaaagtaaagtgattaacagcgcattagagctgcttaatggggtcggaatcgaaggtttaacaacccgtaaactcgcccagaagctaggtgtagagcagcctacattgtattggcatgtaaaaaataagcgggctttgctcgacgccttacccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcactttactttaggttgcgtattggaagaacaagagcatcaagtcgctaaagaagaaagggaaacacctactactgatagtatgccgccattattacgacaagctatcgaattatttgatcgccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 71 - DNA, 1008 bp, synthetic construct
  source 1..1008: mol_type = other DNA
atgtctagattagataaaagtaaagtgattaacagcgcattagagctgcttaatggggtcggaatcgaaggtttaacaacccgtaaactcgcccagaagctaggtgtagagcagcctacattgtattggcatgtaaaaaataagcgggctttgctcgacgccttacccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 72 - DNA, 1008 bp, synthetic construct
  source 1..1008: mol_type = other DNA
atgtctagattagataaaagtaaagtgattaacggcgcattagagctgcttaatggggtcggaatcgaaggtttaacaacccgtaaactcgcccagaagctaggtgtagagcagcctacattgtattggcatgtaaaaaataagcgggctttgctcgacgccttacccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 73 - DNA, 1008 bp, synthetic construct
  source 1..1008: mol_type = other DNA
atgtctagattagataaaagtaaagtgattaacggcgcattagagctgcttaatggggtcggaatcgaaggtttaacaacccgtaaactcgcccagaagctaggtgtagagcagcctacattgtattggcatgtaaaaaataagcgggctttgctcgacgccttacccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcactttactttaggttgcgtattggaagaacaagagcatcaagtcgctaaagaagaaagggaaacacctactactgatagtatgccgccattattacgacaagctatcgaattatttgatcgccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 74 - DNA, 1008 bp, synthetic construct
  source 1..1008: mol_type = other DNA
atgtctagattagataaaagtaaagtgattaacagcgcattagagctgcttaatgaggtcggaatcgaaggtttagcaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcggtggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 75 - DNA, 1008 bp, synthetic construct
  source 1..1008: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatagagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 76 - DNA, 1008 bp, synthetic construct
  source 1..1008: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagaacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 77 - DNA, 1005 bp, synthetic construct
  source 1..1005: mol_type = other DNA
atgtctagattagataaaagtaaagtgattaacagcgcattagagctgcttaatggggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 78 - DNA, 1008 bp, synthetic construct
  source 1..1008: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagaacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtaaaagtgggtccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtag

SEQ ID NO: 79 - skipped

SEQ ID NO: 80 - protein, 335 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKEHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 81 - protein, 334 aa, synthetic construct
MSRLDKSKVINSALELLNGVGIEGLTTRKLAQKLGVEQPTLYWHVKNKRALLDALAIEMLDRHHTHFCPLEGESWQDFLRNNAKSFSALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 82 - protein, 335 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKEHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCKSGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 83 - protein, 335 aa, synthetic construct
MSRLDKSKVINSALELLNEVGIEGLTTRKLAQKLGVEQPTLYWHVKNKRALLDALAIEMLDRHHTHFCPLKGESWQDFLRNNAKSFRCALLSHRNGAKVHSDTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 84 - protein, 335 aa, synthetic construct
MSRLDKSKVINGALELLNGVGIEGLTTRKLAQKLGVEQPTLYWHVKNKRALLDALPIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 85 - skipped

SEQ ID NO: 86 - DNA, 621 bp, synthetic construct
  source 1..621: mol_type = other DNA
atgtctagattagataaaagtaaagtgattaacagcgcattagagctgcttaatgaggtcggaatcgaaggtttaacaacccgtaaactcgcccagaagctaggtgtagagcagcctacactgtattggcatgtaaaaaataagcgggctttgctcgacgccttagccattgagatgttagataggcaccatactcacttttgccctttaaaaggggaaagctggcaagattttttacgcaataaggctaaaagttttagatgtgctttactaagtcatcgcaatggagcaaaagtacattcagatacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcc

SEQ ID NO: 87 - DNA, 22 bp, synthetic construct
  source 1..22: mol_type = other DNA
cagtgagcgagcgagcgcgcag

SEQ ID NO: 88 - skipped

SEQ ID NO: 89 - DNA, 21 bp, synthetic construct
  source 1..21: mol_type = other DNA
gatggagttggccactccctc

SEQ ID NO: 90 - protein, 452 aa, Homo sapiens
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 91 - protein, 338 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 92 - protein, 252 aa, synthetic construct
MIKIATRKYLGKQNVYDIGVERDHNFALKNGFIASNCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 93 - protein, 366 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 94 - protein, 224 aa, synthetic construct
MIKIATRKYLGKQNVYDIGVERDHNFALKNGFIASNCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 95 - protein, 385 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 96 - protein, 205 aa, synthetic construct
MIKIATRKYLGKQNVYDIGVERDHNFALKNGFIASNCHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 97 - protein, 435 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 98 - protein, 155 aa, synthetic construct
MIKIATRKYLGKQNVYDIGVERDHNFALKNGFIASNCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIK

SEQ ID NO: 99 - protein, 250 aa, Homo sapiens
MEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 100 - protein, 22 aa, synthetic construct
GSGATNFSLLKQAGDVEENPGP

SEQ ID NO: 101 - protein, 724 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 102 - protein, 610 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 103 - protein, 524 aa, synthetic construct
MIKIATRKYLGKQNVYDIGVERDHNFALKNGFIASNCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 104 - protein, 638 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 105 - protein, 496 aa, synthetic construct
MIKIATRKYLGKQNVYDIGVERDHNFALKNGFIASNCTQYIRHGSKPMYTPEPDICHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 106 - protein, 657 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 107 - protein, 477 aa, synthetic construct
MIKIATRKYLGKQNVYDIGVERDHNFALKNGFIASNCHELLGHVPLFSDRSFAQFSQEIGLASLGAPDEYIEKLATIYWFTVEFGLCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 108 - protein, 707 aa, synthetic construct
MSTAVLENPGLGRKLSDFGQETSYIEDNCNQNGAISLIFSLKEEVGALAKVLRLFEENDVNLTHIESRPSRLKKDEYEFFTHLDKRSLPALTNIIKILRHDIGATVHELSRDKKKDTVPWFPRTIQELDRFANQILSYGAELDADHPGFKDPVYRARRKQFADIAYNYRHGQPIPRVEYMEEEKKTWGTVFKTLKSLYKTHACYEYNHIFPLLEKYCGFHEDNIPQLEDVSQFLQTCTGFRLRPVAGLLSSRDFLGGLAFRVFHCTQYIRHGSKPMYTPEPDICHELLGHVPLFSDRSFAQFSQEIGLASLGAPDEYIEKLATIYWFTVEFGLCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPNGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 109 - protein, 427 aa, synthetic construct
MIKIATRKYLGKQNVYDIGVERDHNFALKNGFIASNCKQGDSIKAYGAGLLSSFGELQYCLSEKPKLLPLELEKTAIQNYTVTEFQPLYYVAESFNDAKEKVRNFAATIPRPFSVRYDPYTQRIEVLDNTQQLKILADSINSEIGILCSALQKIKGSGATNFSLLKQAGDVEENPGPMEKGPVRAPAEKPRGARCSNGFPERDPPRPGPSRPAEKPPRPEAKSAQPADGWKGERPRSEEDNELNLPNLAAAYSSILSSLGENPQRQGLLKTPWRAASAMQFFTKGYQETISDVLNDAIFDEDHDEMVIVKDIDMFSMCEHHLVPFVGKVHIGYLPNKQVLGLSKLARIVEIYSRRLQVQERLTKQIAVAITEALRPAGVGVVVEATHMCMVMRGVQKMNSKTVTSTMLGVFREDPKTREEFLTLIRS

SEQ ID NO: 110 - protein, 246 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKYSDLELK

SEQ ID NO: 111 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 112 - protein, 373 aa, Homo sapiens
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 113 - protein, 154 aa, synthetic construct
MTTSASSHLNKGIKQVYMSLPQGEKVQAMYIWIDGTGEGLRCKTRTLDSEPKCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 114 - protein, 357 aa, synthetic construct
MIKIATRKYLGKQNVYDIGVERDHNFALKNGFIASNCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 115 - protein, 218 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 116 - protein, 293 aa, synthetic construct
MIKIATRKYLGKQNVYDIGVERDHNFALKNGFIASNCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 117 - protein, 284 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 118 - protein, 227 aa, synthetic construct
MIKIATRKYLGKQNVYDIGVERDHNFALKNGFIASNCLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 119 - protein, 330 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 120 - protein, 181 aa, synthetic construct
MIKIATRKYLGKQNVYDIGVERDHNFALKNGFIASNCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 121 - protein, 353 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 122 - protein, 158 aa, synthetic construct
MIKIATRKYLGKQNVYDIGVERDHNFALKNGFIASNCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 123 - protein, 279 aa, Homo sapiens
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 124 - protein, 144 aa, synthetic construct
MPVAGSELPRRPLPPAAQERDAEPRPPHGELQYLGQIQHILRCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 125 - protein, 273 aa, synthetic construct
MIKIATRKYLGKQNVYDIGVERDHNFALKNGFIASNCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 126 - protein, 262 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 127 - protein, 155 aa, synthetic construct
MIKIATRKYLGKQNVYDIGVERDHNFALKNGFIASNCHALCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 128 - protein, 266 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 129 - protein, 151 aa, synthetic construct
MIKIATRKYLGKQNVYDIGVERDHNFALKNGFIASNCQFYVVNSELSCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 130 - protein, 277 aa, synthetic construct
MPVAGSELPRRPLPPAAQERDAEPRPPHGELQYLGQIQHILRCGVRKDDRTGTGTLSVFGMQARYSLRDEFPLLTTKRVFWKGVLEELLWFIKGSTNAKELSSKGVKIWDANGSRDFLDSLGFSTREEGDLGPVYGFQWRHFGAEYRDMESDLPLMALPPCHALCQFYVVNSELSCLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 131 - protein, 140 aa, synthetic construct
MIKIATRKYLGKQNVYDIGVERDHNFALKNGFIASNCQLYQRSGDMGLGVPFNIASYALLTYMIAHITGLKPGDFIHTLGDAHIYLNHIEPLKIQLQREPRPFPKLRILRKVEKIDDFKAEDFQIEGYNPHPTIKMEMAV

SEQ ID NO: 132 - DNA, 546 bp, synthetic construct
  source 1..546: mol_type = other DNA
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatgtaagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 133 - DNA, 546 bp, synthetic construct
  source 1..546: mol_type = other DNA
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 134 - DNA, 350 bp, synthetic construct
  source 1..350: mol_type = other DNA
actagtattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtttatataagcagagctcgtttagtgaaccgtcagatcgcctggagacgccatccacgctgttttgacctccatagaaga

SEQ ID NO: 135 - DNA, 588 bp, synthetic construct
  source 1..588: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 136 - DNA, 6397 bp, synthetic construct
  source 1..6397: mol_type = other DNA
ggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccgttgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacataacttcgtataaagtatactatacgaagttatcgggcccctctgctaaccatgttcatgccttcttctttttcctacagatgtcagaactcattaaagagaatatgcacatgaagctgtatatggaaggtactgtagacaaccaccatttcaaatgcacgtccgaaggtgaggggaagccatacgagggtacccaaactatgcgcatcaaagtggttgagggtggccccctgccattcgcattcgacatcctggcaactagctttctttacggttccaagacattcataaatcatacccagggtattcccgatttcttcaaacaatccttcccggaagggtttacttgggagcgggtcacgacatatgaagacgggggtgttcttacagccacacaggatacgagtttgcaagacggttgtcttatctataacgtgaagattcggggtgtgaatttcacatccaatggcccggtgatgcagaaaaaaacactgggctgggaagcatttacggagacgttgtatcccgccgatggaggtctcgagggccgaaacgatatggccctcaagttggtaggtggttctcaccttatagcaaacattaagaccacgtatcgatcaaaaaaacccgctaagaatctgaaaatgccaggcgtgtattatgttgattacagactggagcgaataaaagaggctaacaatgagacctacgtcgaacagcatgaagtcgctgtagctagatattgcgacctcccgtcaaagttgggccataaattgaattaacctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcataacttcgtataaagtatactatacgaagttataattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctttttcctacaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttgtaaataaatttagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgt

SEQ ID NO: 137 - DNA, 613 bp, synthetic construct
  source 1..613: mol_type = other DNA
gaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactga

SEQ ID NO: 138 - DNA, 223 bp, synthetic construct
  source 1..223: mol_type = other DNA
atgattaagatcgctacgcggaagtacctggggaaacagaacgtctacgacataggtgtggagcgcgatcacaactttgctctgaaaaatggatttatcgccagcaactgtagggagttgatttcagactatgcaccagattgttttgtgttaatagaaatgaatggcaagttagtcaaaactacgattgaagaactcattccactcaaatatacccgaaatt

SEQ ID NO: 139 - DNA, 2005 bp, synthetic construct
  source 1..2005: mol_type = other DNA
gcttttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagccgcgaccggccaaggtttaatgataggctgcaacgggatgttgggaatatgttgcactggtccgtgagggtaccaacttgtttattgcagcttataatggttacaaataaagcaatagcatcacaaatttcacaaataaagcatttttttcactgcattctagttgtggtttgtccaaactcatcaatgtatcttatcatgtctgaccggttcacttgagctcgagatctgagtacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcggcttaagggttcgatcctctagagtccggaggctggatcggtcccggtgtctactatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatggtacctgcggaggatgccgaggataaccttgttactagcctccgcctggccgttggactgtggataatatggcgtagaggatcctctgcgcgctcgctcgctcactgaggccgcccgggcaaagcccgggcgtcgggcgacctttggtcgcccggcctcagtgagcgagcgagcgcgcagagaa

SEQ ID NO: 140 - protein, 102 aa, Nostoc punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 141 - protein, 36 aa, synthetic construct
MIKIATRKYLGKQNVYDIGVERDHNFALKNGFIASN

SEQ ID NO: 142 - protein, 373 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASICIPRTVGQEKKGYFEDRRPSANCDPFSVTEALIRTCLLNETGDEPFQYKN

SEQ ID NO: 143 - protein, 373 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASISIPRTVGQEKKGYFEDRRPSANCDPFSVTEALIRTCLLNETGDEPFQYKN

SEQ ID NO: 144 - protein, 373 aa, synthetic construct
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRCPSANCDPFSVTEALIRTCLLNETGDEPFQYKN

SEQ ID NO: 145 - DNA, 98 bp, Adeno-associated virus 2
  source 1..98: mol_type = other DNA
ggaggggtggagtcgtgacgtgaattacgtcatagggttagggaggtcctgtattagaggtcacgtgagtgttttgcgacattttgcgacaccatgtg

SEQ ID NO: 146 - DNA, 2366 bp, synthetic construct
  source 1..2366: mol_type = other DNA
cactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagaga

SEQ ID NO: 147 - DNA, 8271 bp, synthetic construct
  source 1..8271: mol_type = other DNA
ccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcactagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcac

SEQ ID NO: 148 - DNA, 2280 bp, synthetic construct
  source 1..2280: mol_type = other DNA
tctctgcgcgctcgctcgctcactgaggccgggcgaccaaaggtcgcccgacgcccgggctttgcccgggcggcctcagtgagcgagcgagcgcgcagaggatccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcactgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 149 - DNA, 8195 bp, synthetic construct
  source 1..8195: mol_type = other DNA
agcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcccgccgggtaactcacggggtatccatgtccatttctgcggcatccagccaggatacccgtcctcgctgacgtaatatcccagcgccgcaccgctgtcattaatctgcacaccggcacggcagttccggctgtcgccggtattgttcgggttgctgatgcgcttcgggctgaccatccggaactgtgtccggaaaagccgcgacgaactggtatcccaggtggcctgaacgaacagttcaccgttaaaggcgtgcatggccacaccttcccgaatcatcatggtaaacgtgcgttttcgctcaacgtcaatgcagcagcagtcatcctcggcaaactctttccatgccgcttcaacctcgcgggaaaaggcacgggcttcttcctccccgatgcccagatagcgccagcttgggcgatgactgagccggaaaaaagacccgacgatatgatcctgatgcagctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgagcttctgatagactaattgacatcatttgagtcaattccagacatgataagatacattgatgagtttggacaaaccacaactagaatgcagtgaaaaaaatgctttatttgtgaaatttgtgatgctattgctttatttgtaaccattataagctgcaataaacaagtttagttaacgcatgatacaaaggcattaaagcagcgtatccacatagcgtaaaaggagcaacatagttaagaataccagtcaatctttcacaaattttgtaatccagaggttgatttcaggcaccgggcttgcgggtcatgcactcagtttggaaggttgtcgactctcatcagatccagctctcgttcaaaaatttcgtcaattggcagcatctggccgtccacggtcataaacttatgatctttagtagcgcggatgagggatccgtcctcgagacagtattcaaaaacttcctgctctcctctgtcgtgccattgagccacgggctgtgtgtagatgttcccattattatcgactgaatagacggtacattcaatcctcttctccacgattttgccgataggaagcaatccgtactcgacagtcaggatctcggtctcgtatgaaaggcacataccacaaggacttaccactcgaatacttctatctacttcgtcagaataagggtgtctaacagctacaatcgtgtcaaaatccttttgtccattcgaaactgcactaccaatcgcaatggcttctgcacaaacagttactcgtcctatatacgcttcaatatgtactgccgaaatgatttctcctgttttcgtacgaattgccgctcccacatgatgtttattatcctcataaagcattgtaatcttctctgtcgctacttctactaattctagatcctgttgagaaatgttaaatgttttcatggtggcgatatcgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagcgatcgcagatccttgcggccgcggatccgatttaaattcgaattcgctagctctagtgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctctgcgcgctcgctcgctcactgaggccgggcgaccaaaggtcgcccgacgcccgggctttgcccgggcggcctcagtgagcgagcgagcgcgcagaggatccgggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcactgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagataacgcaggaaagaaataaaagttttgttactttatagaagaaattttgagtttttgttttttttaataaataaataaacataaataaattgtttgttgaatttattattagtatgtaagtgtaaatataataaaacttaatatctattcaaattaataaataaacctcgatatacagaccgataaaacacatgcgtcaattttacacatgattatctttaacgtacgtcacaatatgattatctttctagggttaatctagctgcgtgttctgcagcgtgtcgagcatcttcatctgctccatcacgctgtaaaacacatttgcaccgcgagtctgcccgtcctccacgggttcaaaaacgtgaatgaacgaggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaag

SEQ ID NO: 150 - DNA, 2445 bp, synthetic construct
  source 1..2445: mol_type = other DNA
tctctgcgcgctcgctcgctcactgaggccgggcgaccaaaggtcgcccgacgcccgggctttgcccgggcggcctcagtgagcgagcgagcgcgcagaggatccaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcaccgctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggtacccaggggtggcgggtgggtgggctgagcacagtgtggcaggcagccgggccccagtgcccacctgcccttcttcatctgccctaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcactgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 151 - DNA, 8359 bp, synthetic construct
  source 1..8359: mol_type = other DNA
gtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcccgccgggtaactcacggggtatccatgtccatttctgcggcatccagccaggatacccgtcctcgctgacgtaatatcccagcgccgcaccgctgtcattaatctgcacaccggcacggcagttccggctgtcgccggtattgttcgggttgctgatgcgcttcgggctgaccatccggaactgtgtccggaaaagccgcgacgaactggtatcccaggtggcctgaacgaacagttcaccgttaaaggcgtgcatggccacaccttcccgaatcatcatggtaaacgtgcgttttcgctcaacgtcaatgcagcagcagtcatcctcggcaaactctttccatgccgcttcaacctcgcgggaaaaggcacgggcttcttcctccccgatgcccagatagcgccagcttgggcgatgactgagccggaaaaaagacccgacgatatgatcctgatgcagctagattaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcagccataccaatgggccctaaaaaaggaatccagtcaattccggggctaaacctggctgccactgtttctttagggacttcgttcctgtgaggacaggaaaccctgcaacctggttgaccagagcaagggatataagaacccagtttgggaacactgctgtttaaatattaaacagggatgtcttgtcccccggaagggagaccagatgagcttctgatagactaattgacatcatttgagtcaattccagacatgataagatacattgatgagtttggacaaaccacaactagaatgcagtgaaaaaaatgctttatttgtgaaatttgtgatgctattgctttatttgtaaccattataagctgcaataaacaagtttagttaacgcatgatacaaaggcattaaagcagcgtatccacatagcgtaaaaggagcaacatagttaagaataccagtcaatctttcacaaattttgtaatccagaggttgatttcaggcaccgggcttgcgggtcatgcactcagtttggaaggttgtcgactctcatcagatccagctctcgttcaaaaatttcgtcaattggcagcatctggccgtccacggtcataaacttatgatctttagtagcgcggatgagggatccgtcctcgagacagtattcaaaaacttcctgctctcctctgtcgtgccattgagccacgggctgtgtgtagatgttcccattattatcgactgaatagacggtacattcaatcctcttctccacgattttgccgataggaagcaatccgtactcgacagtcaggatctcggtctcgtatgaaaggcacataccacaaggacttaccactcgaatacttctatctacttcgtcagaataagggtgtctaacagctacaatcgtgtcaaaatccttttgtccattcgaaactgcactaccaatcgcaatggcttctgcacaaacagttactcgtcctatatacgcttcaatatgtactgccgaaatgatttctcctgttttcgtacgaattgccgctcccacatgatgtttattatcctcataaagcattgtaatcttctctgtcgctacttctactaattctagatcctgttgagaaatgttaaatgttttcatggtggcgatatcgtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagcgatcgcagatccttgcggccgcggatccgatttaaattcgaattcgctagctctagtgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattctctgcgcgctcgctcgctcactgaggccgggcgaccaaaggtcgcccgacgcccgggctttgcccgggcggcctcagtgagcgagcgagcgcgcagaggatccaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcaccgctagcgccgccaccatgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggtacccaggggtggcgggtgggtgggctgagcacagtgtggcaggcagccgggccccagtgcccacctgcccttcttcatctgccctaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtgaaataaaggaaatttattttcattgcaatagtgtgttggaattttttgtgtctctcactgcagaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagataagcaggaaagaaataaaagttttgttactttatagaagaaattttgagtttttgttttttttaataaataaataaacataaataaattgtttgttgaatttattattagtatgtaagtgtaaatataataaaacttaatatctattcaaattaataaataaacctcgatatacagaccgataaaacacatgcgtcaattttacacatgattatctttaacgtacgtcacaatatgattatctttctagggttaatctagctgcgtgttctgcagcgtgtcgagcatcttcatctgctccatcacgctgtaaaacacatttgcaccgcgagtctgcccgtcctccacgggttcaaaaacgtgaatgaacgaggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacga

SEQ ID NO: 152 - DNA, 2477 bp, synthetic construct
  source 1..2477: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgctagcgcctagaagtaaaggcaacatccactgaggagcagttctttgatttgcaccaccaccggatccgggacctgaaataaaagacaaaaagactaaacttacgctagccggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagaga

SEQ ID NO: 153 - DNA, 8377 bp, synthetic construct
  source 1..8377: mol_type = other DNA
gcgcctagaagtaaaggcaacatccactgaggagcagttctttgatttgcaccaccaccggatccgggacctgaaataaaagacaaaaagactaaacttacgctagccggtgcacaccaatgtggtgaatggtcaaatggcgtttattgtatcgagctaggcacttaaatacaatatctctgcaatgcggaattcagtggttcgtccaatccatgtcagacccgtctgttgccttcctaataaggcacgatcgtaccaccttacttccaccaatcggcatgcacggtgctttttctctccttgtaaggcatgttgctaactcatcgttaccatgttgcaagactacaagagtattgcataagactacattggatcctctgcgcgctcgctcgctcactgaggccgcccgggcaaagcccgggcgtcgggcgacctttggtcgcccggcctcagtgagcgagcgagcgcgcagagaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcactagagctagcgaattcgaatttaaatcggatccgcggccgcaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgatatcgccaccatgaaaacatttaacatttctcaacaggatctagaattagtagaagtagcgacagagaagattacaatgctttatgaggataataaacatcatgtgggagcggcaattcgtacgaaaacaggagaaatcatttcggcagtacatattgaagcgtatataggacgagtaactgtttgtgcagaagccattgcgattggtagtgcagtttcgaatggacaaaaggattttgacacgattgtagctgttagacacccttattctgacgaagtagatagaagtattcgagtggtaagtccttgtggtatgtgcctttcatacgagaccgagatcctgactgtcgagtacggattgcttcctatcggcaaaatcgtggagaagaggattgaatgtaccgtctattcagtcgataataatgggaacatctacacacagcccgtggctcaatggcacgacagaggagagcaggaagtttttgaatactgtctcgaggacggatccctcatccgcgctactaaagatcataagtttatgaccgtggacggccagatgctgccaattgacgaaatttttgaacgagagctggatctgatgagagtcgacaaccttccaaactgagtgcatgacccgcaagcccggtgcctgaaatcaacctctggattacaaaatttgtgaaagattgactggtattcttaactatgttgctccttttacgctatgtggatacgctgctttaatgcctttgtatcatgcgttaactaaacttgtttattgcagcttataatggttacaaataaagcaatagcatcacaaatttcacaaataaagcatttttttcactgcattctagttgtggtttgtccaaactcatcaatgtatcttatcatgtctggaattgactcaaatgatgtcaattagtctatcagaagctcatctggtctcccttccgggggacaagacatccctgtttaatatttaaacagcagtgttcccaaactgggttcttatatcccttgctctggtcaaccaggttgcagggtttcctgtcctcacaggaacgaagtccctaaagaaacagtggcagccaggtttagccccggaattgactggattccttttttagggcccattggtatggctgatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctagggttaatctagctgcatcaggatcatatcgtcgggtcttttttccggctcagtcatcgcccaagctggcgctatctgggcatcggggaggaagaagcccgtgccttttcccgcgaggttgaagcggcatggaaagagtttgccgaggatgactgctgctgcattgacgttgagcgaaaacgcacgtttaccatgatgattcgggaaggtgtggccatgcacgcctttaacggtgaactgttcgttcaggccacctgggataccagttcgtcgcggcttttccggacacagttccggatggtcagcccgaagcgcatcagcaacccgaacaataccggcgacagccggaactgccgtgccggtgtgcagattaatgacagcggtgcggcgctgggatattacgtcagcgaggacgggtatcctggctggatgccgcagaaatggacatggataccccgtgagttacccggcgggcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcctcgttcattcacgtttttgaacccgtggaggacgggcagactcgcggtgcaaatgtgttttacagcgtgatggagcagatgaagatgctcgacacgctgcagaacacgcagctagattaaccctagaaagataatcatattgtgacgtacgttaaagataatcatgtgtaaaattgacgcatgtgttttatcggtctgtatatcgaggtttatttattaatttgaatagatattaagttttattatatttacacttacatactaataataaattcaacaaacaatttatttatgtttatttatttattaaaaaaaacaaaaactcaaaatttcttctataaagtaacaaaacttttatttctttcctgcgttatctttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctctgcagtgagagacacaaaaaattccaacacactattgcaatgaaaataaatttcctttatttcacagcagctgtctcaaggctgggtccctcaaaggggcgtcccagcgcggggcctccctgcgcaaacacttggtacccctggctgcgcagcggaagccagcaggacagcagtggcgccgatcagcacaacagacgccctggcggtagggacagcaggcccagccctgtcggttgtctcggcagcaggtctggttatcatggcagaagtgtccttccccacactccacgtccttcacacccacgtgagggctacgggccaggaaggtggcaggctgggcagagaccacttccttctcgcaggatcgagccttcacgttgcaggtgtagccagccgggcagcagtgctggcgatcctcgcagcacacagcatggggcaactggcagcaggcccagctcccacccaggctcgggcagcaggtctgccccaccgggcagctggtgtgctggtcacagccgatgtctctggggtgggataaggaagcccggcgggcaggcatcttctccagtccagccacgatctcgcttcctcgctgacactgcccctcagctacacacgtgtagccctgggggcagcagtgctggtggtccgagcagcagacagcctctgggattggacagcagccccactccccagacgtgagttggcagcaggtatcggaggagggacagctgctgacattatcacaggggacatctctcttcaaggcttgtgggtctggcaggctgaggtgagctggggccttctccatccagggcacctggtggggcccctgttcacaggtacccttctgcgtgtcacacgtaaaccccgcgggacagcagtgtatgtggtcctcacagcacacagcctgggtaaaagggcagcagccccaggcccccgactgtagacggcagcaggtatagccatctgggcagctcacctccatgtcacatttcacatcccccactgtgtgcgcaggcagcttagtgaggaggtccgtggtagcgttctccttggagaggcacttactctggatcaggtcacacacagtgtcttgggggcagcagtgcaggtgatcggagcagcaggtggcgttgggcattgggcagcagccatacttcccactgggcagctcacagcaggtagaaccatcagggcaccgggaccgtgcgtccggacacatgaccgagctggacaaggccactgccctgttagtcctctgggcagggagcttctttgccagggggtgggtgcccgtgggtgtgatgcagcgggtgtgaaccaggtcgcagaaggcaccgtgcggacagcagtgcaccctgtcttcacagcaggaagcctggggcatggggcagcacccccaggagccatcgaccataacacagcacgtggagaagtccgggcattcgaactgactatcagggcactggatggcacccacggagttgttacctgatctttggaagcaggatcgcccgtctgcactgcagtggaagccccgtgggcagcagtgatggccatccccgcatgccacggcctctgggaaggggcagcaactggaagtccctgagacggtaaagatgcaggagtggccggcagagcagtgggcatcaacctggcaggggccacccagatgcctgctcagtgttgtgggccatttgtccagaaggggacggcagcagctgtagctggctcctccggggtccaggcagcaggccacagggcagaactgaccatctgggcaccgcgttccagccaccagccctgctgttaaggccacccagctcaccagggtccacatggtggcggcgcta

SEQ ID NO: 154 - RNA, 36 nt, synthetic construct
  source 1..36: mol_type = other RNA
ggacgaccggggttcgaaccccggatccggccgtcc

SEQ ID NO: 155 - RNA, 158 nt, synthetic construct
  source 1..158: mol_type = other RNA
agcgggcactcttccgtggtctggtggataaattcgcaagggtatcatggcggacgaccggggttcgacccggtccggccgtccgccgtgatccatgcggttaccgcccgcgtgtcgaacccaggtgtgcgacgtcagacaacgggggagcgctcctt